(19) United States
(12) Patent Application Publication
(10) Pub. No.: US 2024/0391875 A1
(43) Pub. Date: Nov. 28, 2024

(54) ISOQUINOLINONES AND QUINOLINONES AS MODULATORS OF POLRMT

(71) Applicant: Pretzel Therapeutics, Inc., Waltham, MA (US)

(72) Inventors: Gabriel Martinez Botella, Wayland, MA (US); Jeremy Green, Waltham, MA (US); Paul S. Charifson, Carrabassett Valley, ME (US); Simon Giroux, Cambridge, MA (US); Andrew Griffin, L'Île Bizard (CA)

(73) Assignee: Pretzel Therapeutics, Inc., Waltham, MA (US)

(21) Appl. No.: 18/687,692

(22) PCT Filed: Aug. 30, 2022
(86) PCT No.: PCT/US2022/042097

§ 371 Date: Feb. 28, 2024

Related U.S. Application Data
(60) Provisional application No. 63/238,750, filed on Aug. 30, 2021.
(60) Provisional application No. 63/276,245, filed on Nov. 5, 2021.
(60) Provisional application No. 63/284,866, filed on Dec. 1, 2021.
(60) Provisional application No. 63/358,727, filed on Jul. 6, 2022.

(51) Int. Cl. C07D 217/26; A61K 31/4725; A61K 31/497; A61K 31/5377; A61K 31/5386; A61K 31/541; C07B 59/00; C07D 217/24; C07D 401/12; C07D 413/12; C07D 417/12; C07D 487/08; C07D 498/08

(52) U.S. Cl. CPC C07D 217/26; A61K 31/4725; A61K 31/497; A61K 31/5377; A61K 31/5386; A61K 31/541; C07B 59/004; C07D 217/24; C07D 401/12; C07D 413/12; C07D 417/12; C07D 487/08; C07D 498/08; C07B 2200/05; C07B 2200/07; C07B 2200/09

(57) ABSTRACT

The present invention provides novel isoquinolinone and quinolinone compounds that are inhibitors of mitochondrial RNA polymerase for treating various diseases such as cancer and others associated with metabolic disorders and mitochondrial dysfunction.

### FIELD OF THE INVENTION

The present invention relates to novel POLRMT modulators, their prodrugs, their pharmaceutically acceptable salts, and pharmaceutical compositions thereof. The present invention also relates to methods of using such compounds and compositions, including to inhibit or promote POLRMT, and to treat various neurodegenerative and metabolic disorders, cancer, and also disorders related to aging and mitochondrial diseases.

### BACKGROUND OF THE INVENTION

Human mitochondrial RNA polymerase, POLRMT (also referred to as h-mtRNAP), is a nuclear-encoded single-subunit DNA-dependent RNA polymerase. POLRMT is 1230 amino acids in length and consists of three distinct regions: (1) a C-terminal polymerase domain (CTD) (residues 648-1230); (2) an N-terminal domain (NTD) (residues 369-647); and (3) an N-terminal extension (NTE) (residues 1-368). See, e.g., Arnold, J. J., et al., “Human mitochondrial RNA polymerase: Structure-function, mechanism and inhibition,” Biochim. Biophys. Acta, 1819, 948-960 (2012). It is structurally related to the single-subunit RNA polymerase encoded by bacteriophage T7. The CTD is also known as the catalytic domain due to its function of catalyzing nucleotide incorporation into a growing RNA molecule during transcription. This domain is highly conserved across species, whereas by contrast the NTE demonstrates significant sequence variability, suggesting organism-specific roles for this domain of POLRMT. Regarding the POLRMT NTD, structurally it resembles the N-terminal domain (also called the promoter-binding domain) of T7 RNA polymerase. However, for promoter-specific transcription initiation POLRMT requires assistance from additional transcription factors, whereas T7 RNA polymerase does not.

A primary biological role of POLRMT is to transcribe the mitochondrial genome to produce the RNAs needed for expression of mitochondrial DNA (mtDNA). Initiation, elongation, and termination are the three steps of mitochondrial transcription. Each of a light-strand promoter (LSP) and two heavy-strand promoters (HSP-1 and HSP-2) on the mtDNA contains a transcription initiation site. See, e.g., Basu, U., et al., “Structure, mechanism, and regulation of mitochondrial DNA transcription initiation,” J. Biol. Chem., 295(52), 18406-425 (2020). For promoter-specific transcription initiation, POLRMT requires two transcription factors, TFAM (transcription factor A mitochondrial) and TFB2M (transcription factor B mitochondrial). See id. Various models suggest different mechanisms by which the initiation complex structure with POLRMT, TFAM, and TFB2M comes together to cover the promoter DNA for initiation of transcription. In one current model TFAM recruits POLRMT to the promoter site to form a protein-protein pre-initiation complex, to which TFB2M binds to form the initiation complex, which covers the promoter DNA. See id. During initiation, the RNA is elongated to about 8-10 nucleotides in length. Conformational changes occur at that point, including promoter release and displacement of the initiation factors, converting the initiation complex into an elongation complex at which time transcription occurs. See id. 0004 The mitochondrial genome encodes the various subunits of the electron transport chain. See, e.g., Shokolenko, I. N., et al., “Maintenance and expression of mammalian mitochondrial DNA,” Annu. Rev. Biochem., 85, 133-160 (2016). Specifically, transcription of the mitochondrial genome is necessary for the expression of 13 subunits of the oxidative phosphorylation (OXPHOS) system, as well as two rRNAs and 22 tRNAs. See, e.g., Shokolenko, I. N., et al., “Mitochondrial transcription in mammalian cells,” Frontiers in Bioscience, Landmark, 22, 835-853 (2017). Thus, POLRMT is essential for biogenesis of the OXPHOS system, resulting in ATP production. This, in turn, is vital for energy homeostasis in the cell. 0005 Dysregulation of POLRMT and the OXPHOS system have been implicated in various disease states, in particular cancer. Cancer is now the second leading cause of death in the United States, with projections indicating that almost two million new cases will be diagnosed in 2022 and over 600,000 deaths will be the result of cancer. See Siegel, R. L. et al., “Cancer statistics 2022.” CA Cancer J Clin. (72) 7-33 (2022). High rates of OXPHOS have been shown to support growth in cancer cell lines, including in a subset of diffuse large B cell lymphoma cells. See, e.g., DeBeradinis, R. J., “A mitochondrial power play in lymphoma,” Cancer Cell, 22, 423-24 (2012). Noteworthy is the observation that metabolic heterogeneity exists not only between different types of cancer, but also among tumors of the same type. Similarly, in a study using melanoma cell lines representative of various stages of tumor progression and that collectively mimic the mixture of cells found in a tumor, it was found that metastatic cells demonstrated a high OXPHOS capacity. Rodrigues, M. F., et al., “Enhanced OXPHOS, glutaminolysis and β-oxidation constitute the metastatic phenotype of melanoma cells,” Biochem. J. 473: 703-715 (2016). These data suggest mitochondria play a role as cells progress toward metastasis, possibly to provide the energy needed for tumor cell migration and invasion.

Relatedly, overexpression of POLRMT has been linked to multiple types of cancers, suggesting that it plays a role in tumor growth. Supporting this hypothesis is, for example, a study involving acute myeloid leukemia (AML) cells, which are known to have high oxidative phosphorylation and mitochondrial mass, as well as low respiratory chain spare reserve capacity. POLRMT knockdown AML cells demonstrated a reduction in POLRMT levels, decreased oxidative phosphorylation, and increased cell death as compared to control AML cells. See Bralha, F. N., et al., “Targeting mitochondrial RNA polymerase in acute myeloid leukemia,” Oncotarget, 6(35), 37216-228 (2015). In other work, injection into nude mice of a human breast cancer cell line that overexpresses POLRMT resulted in increased tumor growth, independent of tumor angiogenesis, suggesting that POLRMT should be considered a tumor promoter or metabolic oncogene. Salem, A. F., et al. “Mitochondrial biogenesis in epithelial cancer cells promotes breast cancer tumor growth and confers autophagy resistance,” Cell Cycle, 11(22), 4174-80 (2012). Recently, the expression of POLRMT in non-small cell lung cancer (NSCLC) has been examined. See Zhou, T. et al., “The requirement of mitochondrial RNA polymerase for non-small cell lung cancer cell growth,” Cell Death and Disease, 12, 751 (2021). While POLRMT mRNA and protein were detected in normal human lung tissue, their levels were significantly higher in cancer tissue. Similar results were obtained when comparing primary lung epithelial cells to NSCLC cells. Using short hairpin RNA (shRNA) to silence POLRMT mRNA and downregulate POLRMT protein resulted in inhibition of NSCLC cell viability, proliferation, migration, and invasion. Moreover, silencing of POLRMT significantly induced apoptosis activation in both primary and established NSCLC cells. Injection of POLRMT shRNA in an adeno-associated virus construct into tumors effectively inhibited NSCLC xenograft growth in mice. Taken together, these data suggest that POLRMT could be an oncogenic gene for NSCLC.

The development of multidrug resistance (MDR) to numerous cancers is associated with poor prognosis and presents significant challenges in the treatment of this disease. Because such resistance encompasses drugs having different structures and mechanisms of action, identifying and targeting a single biochemical pathway that could re-sensitize MDR cancer cells to established chemotherapy would provide a promising treatment strategy. See Yu, H.-J., “Targeting mitochondrial metabolism and RNA polymerase POLRMT to overcome multidrug resistance in cancer,” Front. Chem., 9:775226 (2021). A main reason for the development of MDR is enhanced drug efflux from and decreased drug accumulation in MDR cells due to ATP-dependent protein transporters that pump drugs out of cells. Inhibiting POLRMT and consequently the production of the proteins essential for the OXPHOS system could compromise ATP production and, in turn, the ATP-dependent efflux of chemotherapeutic agents from cancer cells.

Consistent with the findings that the OXPHOS system and POLRMT may be involved in the etiology of and in some cases overexpressed in some cancers, small-molecule inhibitors of POLRMT have been developed. See, e.g., EP 3 598 972 A1; WO 2019/057821 A1; and WO 2020/188049 A1. Some of these inhibitors have been shown to be useful in inhibiting cancer cell proliferation without affecting control cells. See Bonekamp, N. A., et al., “Small-molecule inhibitors of human mitochondrial DNA transcription,” Nature, 588, 712-716 (2020). The cancer cell toxicity was correlated to a considerable increase in the levels of mono- and diphosphate nucleotides with a concomitant decrease in nucleotide triphosphate levels, all the result of a debilitated OXPHOS system. Similarly, treatment with POLRMT inhibitors caused a decrease in citric-acid cycle intermediates and ultimately cellular amino acid levels, the result of which is a state of severe energy and nutrient depletion. See id. Such inhibitors also produced a decrease in tumor volume in mice with no significant toxicity in control animals. Specifically, mtDNA transcript levels in tumor cells were decreased as compared to transcript levels in differentiated tissue. These data highlight the importance of mtDNA expression in rapidly dividing cells as opposed to post-mitotic tissue, a distinction that may be capitalized on using POLRMT inhibitors that are capable of modulating mtDNA transcription and ultimately the OXPHOS system.

While mitochondria are an emerging target for cancer treatment, the resistance mechanisms induced by chronic inhibition of mitochondrial function are poorly understood. In view of the challenges presented by drug resistance in cancer chemotherapy, the development of such resistance to small molecule inhibitors of POLRMT has been investigated. See Mennuni, M. et al., “Metabolic resistance to the inhibition of mitochondrial transcription revealed by CRISPR-Cas9 screen,” EMBO reports, 23: e53054 1-18 (2022). Using a CRISPR-Cas9 whole-genome screen, loss of genes belonging to von Hippel-Lindau (VHL) and mammalian target of rapamycin complex 1 (mTORC1) were the pathways that caused resistance to acute treatment with a POLRMT inhibitor. See id. at pp. 1-2. Moreover, dose-escalated chronic treatment of cells with this molecule resulted in drug-resistant cells that had increased levels of mtDNA, thereby giving rise to increased levels of mitochondrial transcripts and proteins. See id. at p. 5. The drug-resistant cells maintained higher levels of nucleotide levels, tricarboxylic acid cycle intermediates, and amino acids. See id. at p. 7. Notably, the drug-resistant cells did not have mutations in POLRMT that compromise inhibitor binding to the polymerase. See id. The development of resistance to POLRMT inhibitors underscores the importance and need for the development of other POLRMT inhibitors to understand and treat cancers of varying types.

Alterations in the OXPHOS system also have been implicated in the development of insulin resistance and ultimately Type-2 diabetes. In studies involving apoptosis inducing factor (AIF) knockout mice, a primary OXPHOS defect that produced OXPHOS deficiency revealed an increase in insulin sensitivity and resistance to diabetes and obesity. See Pospisilik, J. A., et al., “Targeted deletion of AIF decreases mitochondrial oxidative phosphorylation and protects from obesity and diabetes,” Cell, 131, 476-91 (2007). Correlated with these phenotypic changes were the metabolic alterations of increased glucose uptake and enhanced fuel utilization. Manipulation of the OXPHOS system with POLRMT modulators affords the potential for further understanding the physiological mechanisms involved in diseases such as diabetes and for the development of novel treatments for intervention of such metabolic disorders.

In addition to its critical role in transcription, POLRMT acts as the primase for mtDNA replication, thus playing a part in the regulation of mtDNA levels. Human mtDNA is a circular double-stranded DNA that is packaged in DNA-protein structures called mitochondrial nucleoids, for which TFAM is the most abundant structural component. See, e.g., Filograna, R., et al., “Mitochondrial DNA copy number in human disease: the more the better?” FEBS Letters, 595, 976-1002 (2021). TFAM facilitates mtDNA compaction, which results in regulating the accessibility of the DNA to cellular replication and transcription components. With respect to mtDNA replication, POLRMT is part of the mtDNA replisome along with the hexameric helicase TWINKLE, the heterotrimeric DNA polymerase gamma (POLγ) and the tetrameric mitochondrial single-stranded DNA-binding protein (mtSSB). See id. Its function in this replisome is to synthesize the RNA primers required for the initiation of the synthesis of both strands of mtDNA. While there may be many mechanisms by which mtDNA levels may be regulated, including modulation of POLRMT, what is known to date is that mtDNA copy number can be manipulated through modulation of TFAM expression.

While the correlation is not completely straightforward, changed levels of mtDNA have been implicated in neurogenerative disorders, cancer, and aging. See e.g., Filograna, R., et al., “Mitochondrial DNA copy number in human disease: the more the better?” FEBS Letters, 595, 976-1002 (2021). Particularly challenging is the attempt to understand the relationship between mtDNA copy number and cancer. It appears that such copy number can correlate with both increased and decreased disease burden. As such, tumor type and stage of disease may be important factors in determining the role of mtDNA copy number in the diagnosis and/or prognosis of cancer. With respect to aging, most data show a reduction in mtDNA levels in the older population. That being said, other study data are inconsistent as to the relationship between mtDNA copy number and longevity. By contrast, there appears to be a clearer correlation between neurodegeneration in Alzheimer's disease and reduction in mtDNA levels. Complicating the understanding of the relationship between mtDNA levels and disease is the role that mtDNA mutations have on various disorders. While accumulation of mtDNA mutations appears to occur in almost all types of cancer, it is unclear whether such mutations are causative of the cancer or merely a by-product of rapid replication in fast-dividing cells. Nonetheless, since POLRMT plays a key role in mtDNA replication, POLMRT modulation may provide an effective mechanism by which to understand various disease states and how to slow or alter the progression of disease.

Mutations affecting POLRMT may also cause human disease. See Oláhová, M., et al., “POLRMT mutations impair mitochondrial transcription causing neurological disease.” Nat. Commun., 12, 1135 (2021). POLRMT variants have been identified in a number of unrelated families. Patients present with multiple phenotypes, including global developmental delay, hypotonia, short stature, and speech/intellectual disability in childhood. POLRMT modulation may provide a mechanism to slow or alter the progression of disease.

POLRMT is of fundamental importance for both expression and replication of the human mitochondrial genome. While aspects of POLRMT biochemistry are known, its full physiological role in mitochondrial gene expression and homeostasis, as well as its underlying impact in the etiology of various disease states, remains unclear. Its dysfunction and/or deregulation impacts mitochondrial metabolism, sometimes through the OXPHOS system, which ultimately contributes to many metabolic, degenerative and age-related diseases such as cancer, diabetes, obesity, and Alzheimer's disease. Pharmacological inhibition of POLRMT is one means by which to gain a further understanding of the role of this polymerase in cell physiology and the development of disease. Regulation of metabolic mechanisms, including oxidative phosphorylation, with POLRMT modulators affords an opportunity for intervention in complex disorders. In view of the numerous and varied roles of POLRMT, the need exists for potent and specific modulators of POLRMT.

### SUMMARY OF THE INVENTION

Provided are compounds, pharmaceutically acceptable salts of the compounds, and prodrugs of the compounds; pharmaceutical compositions comprising the compounds or their salts or prodrugs; and methods of using the compounds, salts of the compounds, prodrugs of the compounds, or pharmaceutical compositions of the compounds, their salts, or their prodrugs to treat various neurodegenerative and metabolic disorders, cancer, and also disorders related to aging and mitochondrial diseases. The compounds and their pharmaceutically acceptable salts are particularly useful as modulators of POLRMT.

In one embodiment, the present invention is directed to a compound, a prodrug thereof, or a pharmaceutically acceptable salt thereof, represented by formula (I):

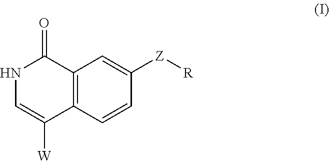

wherein:
Z is O, C(H)(R¹), C₆H₄, C(O), C(O)N(R⁷), or N(R²);
W is C₃-C₈ cycloalkyl, C₄-C₁₂ bicyclic, C₄-C₁₀ cycloalkenyl, C₆-C₁₂ aryl, or 5- to 12-membered heteroaryl, any of which is substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl;
R is hydrogen, or C₁-C₆ alkyl optionally substituted with one or more groups each independently selected from the group consisting of fluoro, hydroxyl, C₁-C₄ alkoxyl, cyano, C₁-C₄ haloalkoxyl, carboxyl, C(O)NR⁵R⁶, NR⁵R⁶ and NR²R³,
or R is C₆-C₁₂ aryl optionally substituted with one or more groups, each independently selected from the group consisting of fluoro, chloro, C₁-C₄ alkyl, trifluoromethyl, difluoromethyl, cyano, hydroxyl, NR⁵R⁶, and C₁-C₄ alkoxyl,
or R is hydroxyl, NR¹R², C(O)R³, C₁-C₄ haloalkoxyl, CH₂CH₂R⁸, CR³R⁴C(O)OR⁵, or 4-7 membered cyclic ring containing one or more heteroatoms, such cyclic ring is optionally substituted with C(O)R¹, NR¹—C(O)—R¹, or CR³R⁴C(O)NR⁵R⁶;
each R¹ is independently hydrogen, C₁-C₄ alkoxyl, or C₁-C₄ alkyl optionally substituted with one or more fluoro groups;
each R² is independently hydrogen, C₁-C₄ alkyl optionally substituted with one or more fluoro groups, C(O)C₁-C₄ alkyl optionally substituted with one or more fluoro or methoxyl groups, C(O)-cycloheteroalkyl optionally substituted with methyl or acetate, or C(O)NHC₁-C₄ alkyl optionally substituted with one or more fluoro groups;
R³ and R⁴ are each independently hydrogen or C₁-C₄ alkyl optionally substituted with one or more groups each independently selected from the group consisting of fluoro, hydroxyl, C₁-C₄ alkoxyl, C₁-C₄ haloalkoxyl, and NR⁵R⁶;
R⁵ and R⁶ are each independently hydrogen, cycloheteroalkyl optionally substituted with acetate, or C₁-C₄ alkyl optionally substituted with one or more groups each independently selected from the group consisting of fluoro, hydroxyl, cyano, C₁-C₄ alkoxyl, C₁-C₄ haloalkoxyl, and NR¹R²,
or if R⁵ and R⁶ are attached to the same nitrogen atom, R⁵ and R⁶ together with their connecting nitrogen form a 3- to 7-membered heterocyclic ring or 6- to 12-membered heterobicyclic ring, each such heterocyclic ring or heterobicyclic ring optionally containing another heteroatom that is N, O, S, S(O), SO₂R¹, or S(O)(NR¹) and each such heterocyclic ring or heterobicyclic ring is optionally substituted with one or more groups each independently selected from the group consisting of fluoro, chloro, and C₁-C₄ alkyl optionally substituted with one or more fluoro, C₁-alkoxyl or hydroxyl, and each such heterocyclic ring or heterobicyclic ring is further optionally substituted with cycloalkyl, cyano, carboxyl, C(O)R¹, C(O)NR¹R², imine, oxo, NR¹—C(O)—R¹, SO₂R¹, or C₁-C₄ alkylcarboxylate;
R⁷ is H or C₁-C₆ alkyl; and
R⁸ is aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R⁸ is OR¹, NR¹R², C(O)R¹, or C(O)NR²R².

In another embodiment, the present invention is directed to a compound, a prodrug thereof, or a pharmaceutically acceptable salt thereof, represented by formula (II):

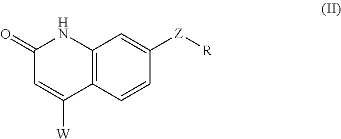

wherein:
Z is O, C(H)(R¹), C₆H₄, C(O), C(O)N(R⁷), or N(R²)
W is C₆-C₁₂ aryl or 5- to 12-membered heteroaryl, either of which is substituted with one or more groups, each independently selected from the group consisting of fluoro, chloro, C₁-C₄ alkyl, trifluoromethyl, difluoromethyl, cyano, hydroxyl, and C₁-C₄ alkoxyl, provided that at least one substituent is at an ortho position relative to the attachment point with the central ring;
R is hydrogen, or C₁-C₆ alkyl optionally substituted with one or more groups each independently selected from the group consisting of fluoro, hydroxyl, C₁-C₄ alkoxyl, cyano, C₁-C₄ haloalkoxyl, C(O)OH, C(O)NR⁵R⁶, NR⁵R⁶ and NR²R³,
or R is C₆-C₁₂ aryl optionally substituted with one or more groups, each independently selected from the group consisting of fluoro, chloro, C₁-C₄ alkyl, trifluoromethyl, difluoromethyl, cyano, hydroxyl, NR⁵R⁶, and C₁-C₄ alkoxyl,
or R is hydroxyl, NR¹R², C₁-C₄ haloalkoxyl, CH₂CH₂R⁸, CR³R⁴C(O)OR⁵, or 4-7 membered cyclic ring containing one or more heteroatoms, such cyclic ring optionally substituted with C(O)R¹, NR¹—C(O)—R¹, or CR³R⁴C(O)NR⁵R⁶;
each R¹ is independently hydrogen, C₁-C₄ alkoxyl, or C₁-C₄ alkyl optionally substituted with one or more fluoro groups;
each R² is independently hydrogen, C₁-C₄ alkyl optionally substituted with one or more fluoro groups, C(O)C_{1,3}-C₄ alkyl optionally substituted with one or more fluoro or methoxyl groups, C(O)-cycloheteroalkyl optionally substituted with methyl or acetate, or C(O)NHC₁-C₄ alkyl optionally substituted with one or more fluoro groups;
R³ and R⁴ are each independently hydrogen or C₁-C₄ alkyl optionally substituted with one or more groups each independently selected from the group consisting of fluoro, hydroxyl, C₁-C₄ alkoxyl, C₁-C₄ haloalkoxyl, and NR⁵R⁶;
R⁵ and R⁶ are each independently hydrogen or C₁-C₄ alkyl optionally substituted with one or more groups each independently selected from the group consisting of fluoro, hydroxyl, cyano, C₁-C₄ alkoxyl, C₁-C₄ haloalkoxyl, and NR¹R²;
or if R⁵ and R⁶ are attached to the same nitrogen atom, R⁵ and R⁶ together with their connecting nitrogen form a 5- or 6-membered heterocyclic ring optionally containing another heteroatom that is N, O, or S, and such heterocyclic ring is optionally substituted with one or more groups each independently selected from the group consisting of fluoro, chloro, and C₁-C₄ alkyl optionally substituted with one or more fluoro, C₁-alkoxyl or hydroxyl, and such heterocyclic ring is further optionally substituted with cycloalkyl, cyano, carboxyl, C(O)R¹, C(O)NR¹R², imine, oxo, SO₂R¹, and C₁-C₄ alkylcarboxylate;
R⁷ is H or C₁-C₆ alkyl; and
R⁸ is aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R⁸ is OR¹, NR¹R², C(O)R¹, or C(O)NR²R².

Further embodiments of the present invention are compounds of the invention (that is, compounds of formula (I), and formula (II)), their pharmaceutically acceptable salts, or prodrugs of the compounds wherein one or more hydrogen is substituted with a deuterium atom.

Additional embodiments of the invention are pharmaceutical compositions comprising a compound of the invention, a pharmaceutically acceptable salt thereof, or a prodrug thereof and one or more pharmaceutically acceptable excipients.

Further embodiments of the invention are methods of treating a disease, such methods comprising administering to a subject in need thereof a therapeutically effective amount of a compound of the invention, a prodrug thereof, or a pharmaceutically acceptable salt thereof. In some embodiments, the disease is selected from the group consisting of adrenal gland cancer, anal cancer, adenocarcinoma, angiosarcoma, bile duct cancer, bladder cancer, blastic plasmacytoid dendritic cell neoplasm, bone cancer, brain cancer, breast cancer, bronchogenic carcinoma, central nervous system (CNS) cancer, cervical cancer, cholangiocarcinoma, chondrosarcoma, colon cancer, choriocarcinoma, colorectal cancer, cancer of connective tissue, esophageal cancer, embryonal carcinoma, fibrosarcoma, gall bladder cancer, gastric cancer, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas), melanoma, Merkel cell carcinoma, mesothelioma, multiple myeloma, muscular cancer, myxosarcoma, neuroblastomas, non-small cell lung cancer, ocular cancer, oral/digestive tract cancer, osteogenic sarcoma, ovarian cancer, papillary carcinoma, pancreatic cancer, polycythemia vera, prostate cancer, rhabdomyosarcoma, renal cancer, retinal cancer, skin cancer, small cell lung carcinoma, stomach cancer, testicular cancer, throat cancer, thyroid cancer, uterine cancer, vaginal cancer, and vulvar cancer. In some embodiments, the disease is selected from the group consisting of Alzheimer's disease and Parkinson's disease. In some embodiments, the disease is selected from the group consisting of obesity, diabetes, non-alcoholic steatohepatitis (NASH), and related metabolic syndromes such as non-alcoholic fatty liver disease (NAFLD). In some embodiments, the disease is related to aging or a mitochondrial disorder.

Additional embodiments of the invention are methods of treating neurodegenerative disorders and metabolic disorders, such as those identified in Bonekamp, N. A. et al. “Small-molecule inhibitors of human mitochondrial DNA transcription,” Nature, 588, 712-716 (2020), Filograna, R. et al, “Mitochondrial DNA copy number in human disease: the more the better?” FEBS Lett., 595, 976-1002 (2021), Wrendenber, A. et al. “Respiratory chain dysfunction in skeletal muscle does not cause insulin resistance,” Biochem. Biophys. Res. Comm., 350, 202-207 (2006), Pospililik, J. A. et al. “Targeted deletion of AIF decreases mitochondrial oxidative phosphorylation and protects from obesity and diabetes,” Cell, 131, 476-491 (2007), and PCT Published International Publication No. WO 2019/057821 A1 and references therein.

Further embodiments of the invention are methods of treating disease of aging.

### DETAILED DESCRIPTION OF THE INVENTION

Modulators of POLRMT are useful in compositions and methods suitable for treating many disorders, such as cancer, neurodegenerative disorders, metabolic disorders, as well as diseases related to aging and mitochondrial diseases. Provided herein are compounds of formula (I), and formula (II), pharmaceutically acceptable salts thereof, prodrugs thereof, and pharmaceutical compositions comprising such compounds, their salts, or their prodrugs that are useful in treating a condition or disease, such as cancer, neurodegenerative disorders, and metabolic disorders.

### Definitions

The term “alkyl” as used herein refers to both branched- and straight-chain saturated aliphatic hydrocarbon groups having the specified number of carbon atoms in a specified range. For example the term “C₁-C₆ alkyl” means linear or branched chain alkyl groups, including all possible isomers, having 1, 2, 3, 4, 5, or 6 carbon atoms. Furthermore, alkyl groups allow for substituents to be located on any of the carbon atoms. For example, a substituted C₃ alkyl group allows for the substituent to be located on any of the three carbon atoms.

The term “alkoxy” or “alkoxyl” as used herein refers to an —O-alkyl group. For example, the term “C₁-C₄ alkoxyl” means-O—C₁-C₄ alkyl. Examples of alkoxyl include methoxyl, ethoxyl, propoxyl (e.g., n-propoxyl and isopropoxyl), and the like.

The term “haloalkoxy” or “haloalkoxyl” as used herein refers to an —O-alkyl group in which at least one of the hydrogen atoms of the alkyl group is replaced with a halogen atom. Examples of haloalkoxyl include trifluoromethoxyl, 2,2,2-trifluoroethoxyl, and the like.

The term “alkanoyl” or “acyl” as used herein refers to an —C(O)-alkyl group. For example, the term “C₁-C₆ alkanoyl” means-C(O)—C₁-C₆ alkyl. Examples of alkanoyl include acetyl, propionyl, butyryl, and the like.

The term “bicyclic” as used herein refers to a saturated or unsaturated 6- to 12-membered ring consisting of two joined cyclic substructures, and includes fused, bridged, and spiro bicyclic rings.

The term “heterobicyclic” as used herein refers to a bicyclic ring that contains 1 or more heteroatom(s) in one or more rings that are optionally substituted or oxidized, and may be selected from nitrogen (including N-oxides), oxygen, sulfur (including oxidized forms such as sulfones and sulfonates), phosphorus (including oxidized forms such as phosphates), boron, etc. Examples of heterobicyclic rings include, but are not limited to 8-azabicyclo[3.2.1]octan-8-yl, 3-oxa-8-azabicyclo[3.2.1]octan-8-yl, 8-oxa-3-azabicyclo[3.2.1]octan-3-yl, and 5-methyl-2,5-diazabicyclo[2.2.1]heptan-2-yl.

The term “cycloalkyl” as used herein refers to a cyclized alkyl ring having the indicated number of carbon atoms in a specified range. Thus, for example, “C₃-C₆ cycloalkyl” encompasses each of cyclopropyl, cyclobutyl, cyclopentyl, and cyclohexyl.

The term “cycloalkenyl” as used herein refers to partially unsaturated monocyclic, bicyclic, tricyclic, or other polycyclic hydrocarbon groups. A ring carbon (e.g., saturated or unsaturated) is the point of attachment of the cycloalkenyl substituent. In certain embodiments, a cycloalkenyl is a C₄-C₁₀ cycloalkenyl. In certain embodiments, a cycloalkenyl is monocyclic, or is bicyclic. Examples of cycloalkenyl, include but are not limited to, cyclopentenyl, cyclohexenyl, cyclohexadienyl, or norbornenyl. In some embodiments, cycloalkenyl includes groups with more than one double bond such as 1,3- and 1,4-cyclohexadienyl.

The term “aryl” as used herein refers to a monocyclic or fused bicyclic ring system having the characteristics of aromaticity, wherein at least one ring contains a completely conjugated pi-electron system. Typically, aryl groups contain 6 to 14 carbon atoms (“C₆-C₁₄ aryl”) or preferably, 6 to 12 carbon atoms (“C₆-C₁₂ aryl”). Fused aryl groups may include an aryl ring (e.g., a phenyl ring) fused to another aryl ring, or fused to a saturated or partially unsaturated carbocyclic or heterocyclic ring. The point of attachment to the base molecule on such fused aryl ring systems may be a C atom of the aromatic portion or a C or N atom of the non-aromatic portion of the ring system. Examples, without limitation, of aryl groups include phenyl, biphenyl, naphthyl, anthracenyl, indanyl, indenyl, and tetrahydronaphthyl.

The term “cycloaryl” herein refers to a polycyclic group wherein an aryl group is fused to a 5- or 6-membered aliphatic ring. For example, “C₆-C₁₂ cycloaryl” means a C₆-C₁₂ aryl fused to a 5- or 6-membered aliphatic ring.

The term “heteroaryl” as used herein refers to (i) a 5- or 6-membered ring having the characteristics of aromaticity containing at least one heteroatom selected from N, O and S, wherein each N is optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatoms, at least one ring is aromatic, each N is optionally in the form of an oxide, and each S in a ring which is not aromatic is optionally S(O) or S(O)₂. Typically, heteroaryl groups contain 5 to 14 ring atoms (“5-14 membered heteroaryl”), and preferably 5 to 12 ring atoms (“5- to 12-membered heteroaryl”). Heteroaryl rings are attached to the base molecule via a ring atom of the heteroaromatic ring, such that aromaticity is maintained. Suitable 5- and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1,2-a]pyridinyl, benzotriazolyl, indazolyl, indolinyl, and isoindolinyl.

The term “heteroaryloxy” or “heteroaryloxyl” as used herein refers to an —O-heteroaryl group.

The term “heterocycle”, “heterocyclyl”, or “heterocyclic” as used herein represents a stable 3- to 10-membered monocyclic, non-aromatic ring that is either saturated or unsaturated, and that consists of carbon atoms and from one to two heteroatoms selected from the group consisting of N, O, and S. Examples include oxiranyl, aziridinyl, oxetanyl, azetidinyl, tetrahydrofuranyl, pyrrolidinyl, tetrahydropyranyl, piperidinyl, 1,4-dioxanyl, morpholinyl, piperazinyl, azepanyl, oxepanyl, and oxazepanyl.

The term “Oxo” as used herein refers to a group which consists of oxygen which is double bonded to carbon or any other element.

The term “imine” as used herein refers to a group containing a carbon-nitrogen double bond.

The term “carboxyl” as used herein refers to a combination of two functional groups attached to a single carbon atom, namely, hydroxyl (OH) and carbonyl (O).

The term “optionally substituted” or “optional substituents” as used herein means that the groups are either unsubstituted or substituted with one or more of the substituents specified. When the groups are substituted with more than one substituent, the substituents may be the same or different. Furthermore, when using the terms “independently,” “independently are,” and “independently selected from” means that the groups may be the same or different.

The term “deuterium” as used herein refers to an isotope of hydrogen that has one proton and one neutron in its nucleus and that has twice the mass of ordinary hydrogen. Deuterium herein is represented by the symbol “D”.

The term “deuterated” by itself or used to modify a compound or group as used herein refers to the presence of at least one deuterium atom attached to carbon. For example, the term “deuterated compound” refers to a compound which contains one or more carbon-bound deuterium(s). In a deuterated compound of the present invention, when a particular position is designated as having deuterium, it is understood that the abundance of deuterium at that position is substantially greater than the natural abundance of deuterium, which is about 0.015%.

The term “undeuterated” or “non-deuterated” as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as “H” or “hydrogen”, the position is understood to have hydrogen at its natural abundance isotopic composition.

The term “isotopic enrichment factor” as used herein refers to the ratio between the isotope abundance and the natural abundance of a specified isotope.

The term “isotopologue” as used herein refers to a species in which the chemical structure differs from a specific compound of the invention only in the isotopic composition thereof.

The term “substantially free of other stereoisomers” as used herein means less than 10% of other stereoisomers, preferably less than 5% of other stereoisomers, more preferably less than 2% of other stereoisomers and most preferably less than 1% of other stereoisomers are present.

The term “pharmaceutically acceptable salt” as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be found in Stahl, P. H. et al. Pharmaceutical Salts: Properties, Selection, and Use, 2nd Revised Edition, Wiley, 2011.

The term “prodrug” as used herein refers to derivatives of compounds of the invention which may have reduced pharmacological activity, but can, when administered to a patient, be converted into the inventive compounds. Design and use of prodrugs may be found in “Pro-drugs as Novel Delivery Systems,” Vol. 14, ACS Symposium Series (T Higuchi and W Stella) and “Bioreversible Carriers in Drug Design,” Pergamon Press, 1987 (ed. E B Roche, American Pharmaceutical Association), the disclosures of which are incorporated herein by reference in their entireties. Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety. Some non-limiting examples of prodrugs in accordance with the invention include: (i) where the compound contains a carboxylic acid functionality —(COOH), an ester thereof, for example, replacement of the hydrogen with (C₁-C₆)alkyl; (ii) where the compound contains an alcohol functionality (—OH), an ether thereof, for example, replacement of the hydrogen with (C₁-C₆)alkanoyloxymethyl, or with a phosphate ether group; and (iii) where the compound contains a primary or secondary amino functionality (—NH₂ or —NHR, where R is not H), an amide thereof, for example, replacement of one or both hydrogens with C₁-C₆ alkanoyl. Further examples of replacement groups in accordance with the foregoing examples and examples of other prodrug types may be found in the aforementioned references.

The terms “treatment”, “treating” and “treat” as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term “therapeutically effective amount” as used herein refers to that amount of compound of the invention that will elicit the biological or medical response of a tissue, system, animal, or human that is being sought by a researcher, veterinarian, medical doctor or other. As will be recognized by a person of ordinary skill in the art, a therapeutically effective amount of the compounds of the invention will vary and will depend on the diseases treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

### Compounds

In one embodiment, the present invention is directed to a compound, a prodrug thereof, or a pharmaceutically acceptable salt thereof, represented by formula (I):

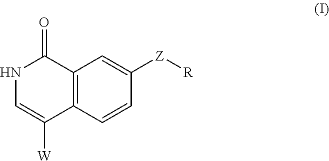

wherein:
Z is O, C(H)(R¹), C₆H₄, C(O), C(O)N(R⁷), or N(R²)
W is C₃-C₈ cycloalkyl, C₄-C₁₂ bicyclic, C₄-C₁₀ cycloalkenyl, C₆-C₁₂ aryl, or 5- to 12-membered heteroaryl, any of which is substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl;
R is hydrogen, or C₁-C₆ alkyl optionally substituted with one or more groups each independently selected from the group consisting of fluoro, hydroxyl, C₁-C₄ alkoxyl, cyano, C₁-C₄ haloalkoxyl, carboxyl, C(O)NR⁵R⁶, NR⁵R⁶ and NR²R³,
or R is C₆-C₁₂ aryl optionally substituted with one or more groups, each independently selected from the group consisting of fluoro, chloro, C₁-C₄ alkyl, trifluoromethyl, difluoromethyl, cyano, hydroxyl, NR⁵R⁶, and C₁-C₄ alkoxyl,
or R is hydroxyl, NR¹R², C(O)R³, C₁-C₄ haloalkoxyl, CH₂CH₂R⁸, CR³R⁴C(O)OR⁵, or 4-7 membered cyclic ring containing one or more heteroatoms, such cyclic ring is optionally substituted with C(O)R¹, NR¹—C(O)—R¹, or CR³R⁴C(O)NR⁵R⁶;
each R¹ is independently hydrogen, C₁-C₄ alkoxyl, or C₁-C₄ alkyl optionally substituted with one or more fluoro groups;
each R² is independently hydrogen, C₁-C₄ alkyl optionally substituted with one or more fluoro groups, C(O)C₁-C₄ alkyl optionally substituted with one or more fluoro or methoxyl groups, C(O)-cycloheteroalkyl optionally substituted with methyl or acetate, or C(O)NHC₁-C₄ alkyl optionally substituted with one or more fluoro groups;
R³ and R⁴ are each independently hydrogen or C₁-C₄ alkyl optionally substituted with one or more groups each independently selected from the group consisting of fluoro, hydroxyl, C₁-C₄ alkoxyl, C₁-C₄ haloalkoxyl, and NR⁵R⁶;
R⁵ and R⁶ are each independently hydrogen, cycloheteroalkyl optionally substituted with acetate, or C₁-C₄ alkyl optionally substituted with one or more groups each independently selected from the group consisting of fluoro, hydroxyl, cyano, C₁-C₄ alkoxyl, C₁-C₄ haloalkoxyl, and NR¹R²,
or if R⁵ and R⁶ are attached to the same nitrogen atom, R⁵ and R⁶ together with their connecting nitrogen form a 3- to 7-membered heterocyclic ring or 6- to 12-membered heterobicyclic ring, each such heterocyclic ring or heterobicyclic ring optionally containing another heteroatom that is N, O, S, S(O), SO₂R¹, or S(O)(NR¹) and each such heterocyclic ring or heterobicyclic ring is optionally substituted with one or more groups each independently selected from the group consisting of fluoro, chloro, and C₁-C₄ alkyl optionally substituted with one or more fluoro, C₁-alkoxyl or hydroxyl, and each such heterocyclic ring or heterobicyclic ring is further optionally substituted with cycloalkyl, cyano, carboxyl, C(O)R¹, C(O)NR¹R², imine, oxo, NR¹—C(O)—R¹, SO₂R¹, or C₁-C₄ alkylcarboxylate;
R⁷ is H or C₁-C₆ alkyl; and
R⁸ is aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R⁸ is OR¹, NR¹R², C(O)R¹, or C(O)NR²R².

In certain embodiments, the present invention is directed to a compound, a prodrug thereof, or a pharmaceutically acceptable salt thereof, represented by formula (I):

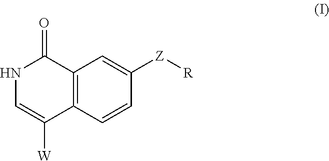

wherein:
Z is O, C(H)(R¹), C₆H₄, C(O), C(O)N(R⁷), or N(R²)
W is C₆ cycloalkyl, C₅ bicyclic, C₆ cycloalkenyl, C₆ aryl or 5-membered heteroaryl, any of which is substituted with one or more groups, each independently selected from the group consisting of fluoro, chloro, cyano, and C₁-C₂ alkyl optionally substituted with one or more deuterium or hydroxyl;
R is hydrogen, or C₁-C₄ alkyl optionally substituted with one or more groups each independently selected from the group consisting of fluoro, hydroxyl, C₁ alkoxyl, cyano, carboxyl, C(O)NR⁵R⁶, and NR²R³,
or R is hydroxyl, NR¹R², C(O)R³, CR³R⁴C(O)OR⁵, or 4-7 membered cyclic ring containing one or more heteroatoms, such cyclic ring is optionally substituted with C(O)R¹, NR¹—C(O)—R¹, or CR³R⁴C(O)NR⁵R⁶;
each R¹ is independently hydrogen, C₁ alkoxyl, or C₁-C₂ alkyl;
each R² is independently hydrogen, C₁-C₂ alkyl optionally substituted with one or more fluoro groups, C(O)C₁-C₄ alkyl optionally substituted with methoxy, or C(O)-cycloheteroalkyl optionally substituted with methyl or acetate;
R³ and R⁴ are each independently hydrogen or C₁ alkyl optionally substituted with C₁ alkoxyl;
R⁵ and R⁶ are each independently hydrogen, C₁-C₂ alkyl, or cycloheteroalkyl substituted with acetate;
or if R⁵ and R⁶ are attached to the same nitrogen atom, R⁵ and R⁶ together with their connecting nitrogen form a 5- or 6-membered heterocyclic ring or 8-membered heterobicyclic ring, each such heterocyclic ring or heterobicyclic ring optionally containing another heteroatom that is N, O, S, S(O), SO₂R¹, or S(O)(NR¹) and each such heterocyclic ring or heterobicyclic ring is optionally substituted with one or more groups each independently selected from the group consisting of fluoro and C₁-C₂ alkyl optionally substituted with one or more fluoro, C₁-alkoxyl or hydroxyl, and each such heterocyclic ring or heterobicyclic ring is further optionally substituted with cyano, carboxyl, C(O)R¹, C(O)NR¹R², NR¹—C(O)—R¹, or SO₂R¹; and
R⁷ is C₁ alkyl.

In certain embodiments, the present invention is directed to a compound, a prodrug thereof, or a pharmaceutically acceptable salt thereof, represented by formula (I):

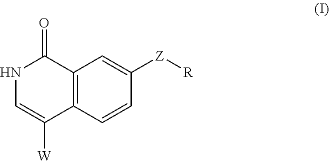

wherein:
Z is O, or C₆H₄;
W is C₆ aryl substituted with one or more groups, each independently selected from the group consisting of fluoro, chloro, and C₁-C₂ alkyl;
R is hydrogen, C₁-C₄ alkyl optionally substituted with one or more groups each independently selected from the group consisting of fluoro, C₁ alkoxyl, cyano, carboxyl, and C(O)NR⁵R⁶;
each R¹ is independently hydrogen or C₁-C₂ alkyl;
each R² is independently hydrogen; R⁵ and R⁶ are each independently hydrogen;
or if R⁵ and R⁶ are attached to the same nitrogen atom, R⁵ and R⁶ together with their connecting nitrogen form a 5- or 6-membered heterocyclic ring or 8-membered heterobicyclic ring, each such heterocyclic ring or heterobicyclic ring optionally containing another heteroatom that is N or O, and each such heterocyclic ring or heterobicyclic ring is optionally substituted with one or more groups each independently selected from the group consisting of fluoro and C₁-C₂ alkyl optionally substituted with one or more fluoro, or hydroxyl, and each such heterocyclic ring or heterobicyclic ring is further optionally substituted with cyano, carboxyl, C(O)R¹, C(O)NR¹R², or SO₂R¹.
   For the compounds of Formula (I), in certain embodiments, Z is O.

In certain embodiments, Z is C₆H₄.

In certain embodiments, Z is C(O).

In certain embodiments, Z is C(O)N(R⁷).

In certain embodiments, Z is N(R²).

In certain embodiments, W is C₆ aryl substituted with one or more groups, each independently selected from the group consisting of fluoro, chloro, C₁-C₂ alkyl, and cyano.

In certain embodiments, W is a 5-membered heteroaryl substituted with one or more groups, each independently selected from the group consisting of fluoro, chloro, C₁-C₂ alkyl, and cyano.

In certain embodiments, R is hydrogen.

In certain embodiments, R is C₁-C₄ alkyl optionally substituted with one or more groups each independently selected from the group consisting of hydroxyl, C₁ alkoxyl, cyano, C(O)OH, C(O)NR⁵R⁶, and NR²R³.

In certain embodiments, R is NR¹R².

In certain embodiments, R is CR³R⁴C(O)OR⁵.

In certain embodiments, R is a 4-7 membered cyclic ring containing one or more heteroatoms, such cyclic ring is optionally substituted with C(O)R¹, or CR³R⁴C(O)NR⁵R⁶.

In certain embodiments, R¹ is independently hydrogen, C₁ alkoxyl, or C₁-C₂ alkyl.

In certain embodiments, R² is independently hydrogen or C₁-C₂ alkyl optionally substituted with C(O)C₁-C₃ alkyl.

In certain embodiments, R³ and R⁴ are each independently hydrogen or C₁ alkyl.

In certain embodiments, R⁵ and R⁶ are each independently hydrogen or C₁-C₂ alkyl.

In certain embodiments, R⁵ and R⁶ are attached to the same nitrogen atom, and R⁵ and R⁶ together with their connecting nitrogen form a 5- or 6-membered heterocyclic ring, and such 5- or 6-membered heterocyclic ring is optionally substituted with one or more groups each independently selected from the group consisting of fluoro, C₁ alkyl, cycloalkyl, cyano, carboxyl, C(O)R¹, C(O)NR¹R², imine, oxo, SO₂R¹, and C₁-C₄ alkylcarboxylate.

In certain embodiments, R⁷ is C₁ alkyl.

In certain embodiments, the compound is (S)-1-(N-methyl-N-(1-oxo-4-(o-tolyl)-1,2-dihydroisoquinolin-7-yl)-D-alanyl)piperidine-3-carboxylic acid, Example 154, or a pharmaceutically acceptable salt thereof:

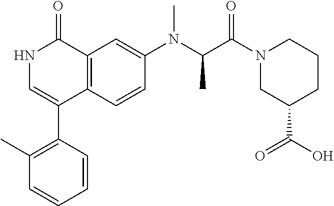 [SMILES: Cc1ccccc1-c1c[nH]c(=O)c2cc(N(C)[C@H](C)C(=O)N3CCC[C@H](C(=O)O)C3)ccc12]

In certain embodiments, the compound is (S)-1-(N-(4-(2-chloro-4-fluorophenyl)-1-oxo-1,2-dihydroisoquinolin-7-yl)-N-methyl-D-alanyl)piperidine-3-carboxylic acid, Example 155, or a pharmaceutically acceptable salt thereof:

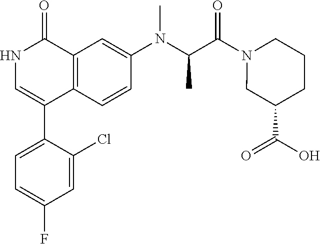 [SMILES: C[C@H](C(=O)N1CCC[C@H](C(=O)O)C1)N(C)c1ccc2c(-c3ccc(F)cc3Cl)c[nH]c(=O)c2c1]

In certain embodiments, the compound is (S)-1-((S)-3-(4-(2-chloro-4-fluorophenyl)-1-oxo-1,2-dihydroisoquinolin-7-yl)-2-methylpropanoyl)piperidine-3-carboxylic acid, Example 156, or a pharmaceutically acceptable salt thereof:

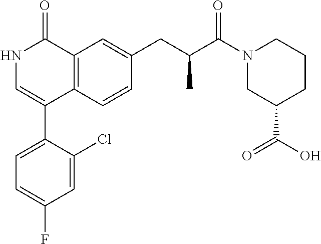 [SMILES: C[C@@H](Cc1ccc2c(-c3ccc(F)cc3Cl)c[nH]c(=O)c2c1)C(=O)N1CCC[C@H](C(=O)O)C1]

In certain embodiments, the compound is (S)-1-((R)-3-(4-(2-chloro-4-fluorophenyl)-1-oxo-1,2-dihydroisoquinolin-7-yl)-2-methylpropanoyl)piperidine-3-carboxylic acid, Example 157, or a pharmaceutically acceptable salt thereof:

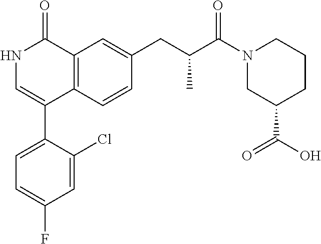 [SMILES: C[C@H](Cc1ccc2c(-c3ccc(F)cc3Cl)c[nH]c(=O)c2c1)C(=O)N1CCC[C@H](C(=O)O)C1]

In certain embodiments, the compound is (S)-4-((R)-2-((4-(2-chloro-4-fluorophenyl)-1-oxo-1,2-dihydroisoquinolin-7-yl)oxy)propanoyl)morpholine-3-carbonitrile, Example 158, or a pharmaceutically acceptable salt thereof:

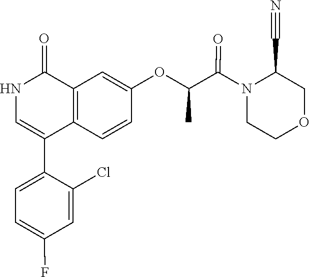 [SMILES: C[C@@H](Oc1ccc2c(-c3ccc(F)cc3Cl)c[nH]c(=O)c2c1)C(=O)N1CCOC[C@@H]1C#N]

In certain embodiments, the compound is (R)-4-((R)-2-((4-(2-chloro-4-fluorophenyl)-1-oxo-1,2-dihydroisoquinolin-7-yl)oxy)propanoyl)morpholine-3-carbonitrile, Example 159, or a pharmaceutically acceptable salt thereof:

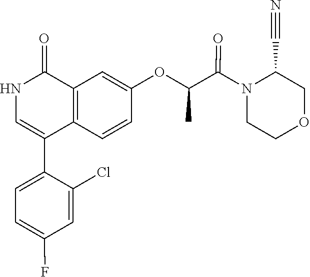 [SMILES: C[C@@H](Oc1ccc2c(-c3ccc(F)cc3Cl)c[nH]c(=O)c2c1)C(=O)N1CCOC[C@H]1C#N]

In certain embodiments, the compound is 3-((4-(2-chloro-4-fluorophenyl)-1-oxo-1,2-dihydroisoquinolin-7-yl)(methyl)amino)propanoic acid, Example 160, or a pharmaceutically acceptable salt thereof:

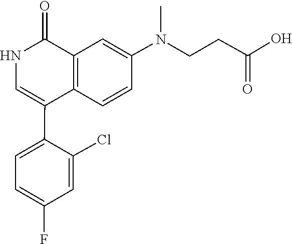 [SMILES: CN(CCC(=O)O)c1ccc2c(-c3ccc(F)cc3Cl)c[nH]c(=O)c2c1]

In certain embodiments, the compound is 3-(methyl(1-oxo-4-(o-tolyl)-1,2-dihydroisoquinolin-7-yl)amino)propanoic acid, Example 161, or a pharmaceutically acceptable salt thereof:

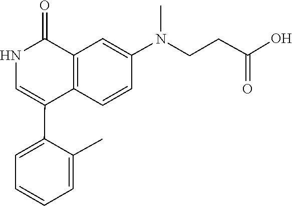 [SMILES: Cc1ccccc1-c1c[nH]c(=O)c2cc(N(C)CCC(=O)O)ccc12]

In certain embodiments, the compound is 3-(methyl(1-oxo-4-(o-tolyl)-1,2-dihydroisoquinolin-7-yl)amino)propanamide, Example 162, or a pharmaceutically acceptable salt thereof:

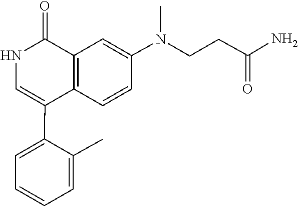 [SMILES: Cc1ccccc1-c1c[nH]c(=O)c2cc(N(C)CCC(N)=O)ccc12]

In certain embodiments, the compound is 3-((4-(2-chloro-4-fluorophenyl)-1-oxo-1,2-dihydroisoquinolin-7-yl)(methyl)amino)propanamide, Example 163, or a pharmaceutically acceptable salt thereof:

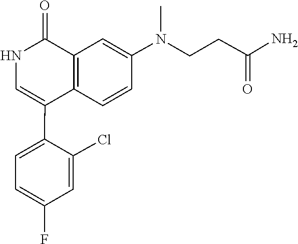 [SMILES: CN(CCC(N)=O)c1ccc2c(-c3ccc(F)cc3Cl)c[nH]c(=O)c2c1]

In certain embodiments, the compound is 2-((4-(2-(hydroxymethyl)-6-methylphenyl)-1-oxo-1,2-dihydroisoquinolin-7-yl)oxy)acetonitrile, Example 164, or a pharmaceutically acceptable salt thereof:

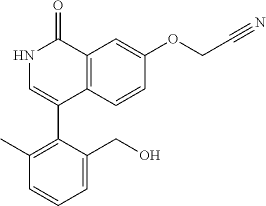 [SMILES: Cc1cccc(CO)c1-c1c[nH]c(=O)c2cc(OCC#N)ccc12]

In certain embodiments, the compound is 2-((4-(2-(hydroxymethyl)phenyl)-1-oxo-1,2-dihydroisoquinolin-7-yl)oxy)acetonitrile, Example 165, or a pharmaceutically acceptable salt thereof:

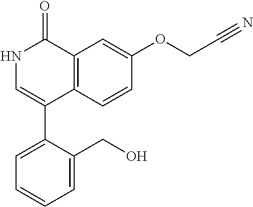 [SMILES: N#CCOc1ccc2c(-c3ccccc3CO)c[nH]c(=O)c2c1]

In certain embodiments, the compound is 2-((4-(2-(1-hydroxyethyl)phenyl)-1-oxo-1,2-dihydroisoquinolin-7-yl)oxy)acetonitrile, Example 166, or a pharmaceutically acceptable salt thereof:

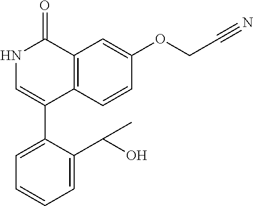 [SMILES: CC(O)c1ccccc1-c1c[nH]c(=O)c2cc(OCC#N)ccc12]

In certain embodiments, the compound is 2-((4-cyclohexyl-1-oxo-1,2-dihydroisoquinolin-7-yl)oxy)acetonitrile, Example 167, or a pharmaceutically acceptable salt thereof:

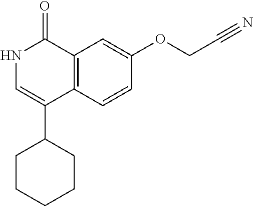 [SMILES: N#CCOc1ccc2c(C3CCCCC3)c[nH]c(=O)c2c1]

In certain embodiments, the compound is 2-((4-(bicyclo[1.1.1]pentan-1-yl)-1-oxo-1,2-dihydroisoquinolin-7-yl)oxy)acetonitrile, Example 168, or a pharmaceutically acceptable salt thereof:

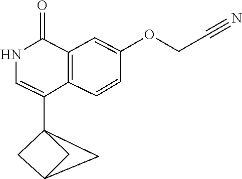 [SMILES: N#CCOc1ccc2c(C34CC(C3)C4)c[nH]c(=O)c2c1]

In certain embodiments, the compound is (S)-5,5-difluoro-1-((R)-2-((1-oxo-4-(o-tolyl)-1,2-dihydroisoquinolin-7-yl)oxy)propanoyl)piperidine-3-carboxylic acid, Example 169, or a pharmaceutically acceptable salt thereof:

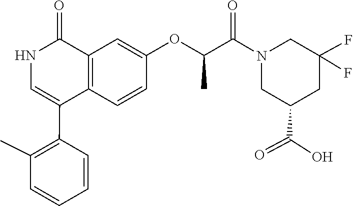 [SMILES: Cc1ccccc1-c1c[nH]c(=O)c2cc(O[C@H](C)C(=O)N3C[C@@H](C(=O)O)CC(F)(F)C3)ccc12]

In certain embodiments, the compound is (S)-3-methyl-1-((R)-2-((1-oxo-4-(o-tolyl)-1,2-dihydroisoquinolin-7-yl)oxy)propanoyl)piperidine-3-carboxylic acid, Example 170, or a pharmaceutically acceptable salt thereof:

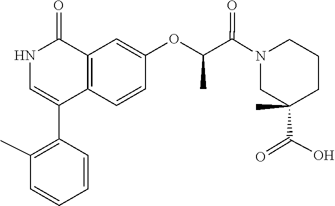 [SMILES: Cc1ccccc1-c1c[nH]c(=O)c2cc(O[C@H](C)C(=O)N3CCC[C@](C)(C(=O)O)C3)ccc12]

In certain embodiments, the compound is (R)-3-methyl-1-((R)-2-((1-oxo-4-(o-tolyl)-1,2-dihydroisoquinolin-7-yl)oxy)propanoyl)piperidine-3-carboxylic acid, Example 171, or a pharmaceutically acceptable salt thereof:

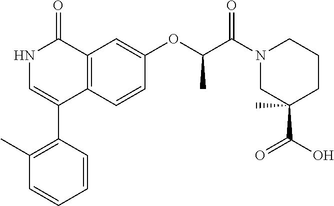 [SMILES: Cc1ccccc1-c1c[nH]c(=O)c2cc(O[C@H](C)C(=O)N3CCC[C@@](C)(C(=O)O)C3)ccc12]

In certain embodiments, the compound is (S)-1-((R)-2-((4-(2-(difluoromethyl)phenyl)-1-oxo-1,2-dihydroisoquinolin-7-yl)oxy)propanoyl)piperidine-3-carboxylic acid, Example 172, or a pharmaceutically acceptable salt thereof:

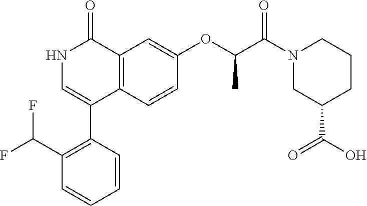 [SMILES: C[C@@H](Oc1ccc2c(-c3ccccc3C(F)F)c[nH]c(=O)c2c1)C(=O)N1CCC[C@H](C(=O)O)C1]

In certain embodiments, the compound is (S)-1-((R)-2-((1-oxo-4-(2-(trifluoromethyl)phenyl)-1,2-dihydroisoquinolin-7-yl)oxy)propanoyl)piperidine-3-carboxylic acid, Example 173, or a pharmaceutically acceptable salt thereof:

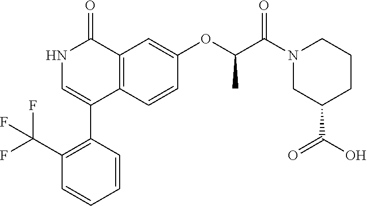 [SMILES: C[C@@H](Oc1ccc2c(-c3ccccc3C(F)(F)F)c[nH]c(=O)c2c1)C(=O)N1CCC[C@H](C(=O)O)C1]

In certain embodiments, the compound is 2-((4-(4-fluoro-2,6-dimethylphenyl)-1-oxo-1,2-dihydroisoquinolin-7-yl)oxy)acetonitrile, Example 174, or a pharmaceutically acceptable salt thereof:

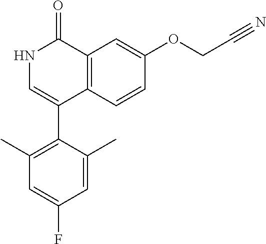 [SMILES: Cc1cc(F)cc(C)c1-c1c[nH]c(=O)c2cc(OCC#N)ccc12]

In certain embodiments, the compound is (R)—N—((R)-1-acetylpyrrolidin-3-yl)-2-((4-(2-chloro-4-fluorophenyl)-1-oxo-1,2-dihydroisoquinolin-7-yl)oxy)-N-methylpropanamide, Example 175, or a pharmaceutically acceptable salt thereof:

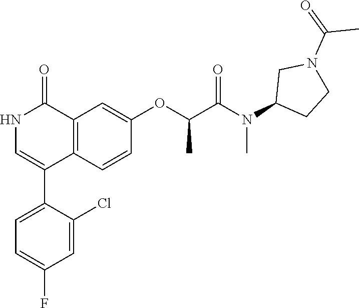 [SMILES: CC(=O)N1CC[C@@H](N(C)C(=O)[C@@H](C)Oc2ccc3c(-c4ccc(F)cc4Cl)c[nH]c(=O)c3c2)C1]

In certain embodiments, the compound is (R)—N—((S)-1-acetylpyrrolidin-3-yl)-2-((4-(2-chloro-4-fluorophenyl)-1-oxo-1,2-dihydroisoquinolin-7-yl)oxy)-N-methylpropanamide, Example 176, or a pharmaceutically acceptable salt thereof:

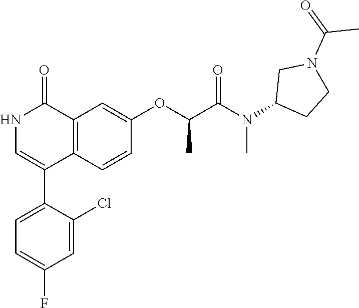 [SMILES: CC(=O)N1CC[C@H](N(C)C(=O)[C@@H](C)Oc2ccc3c(-c4ccc(F)cc4Cl)c[nH]c(=O)c3c2)C1]

In certain embodiments, the compound is (R)-4-(2-chloro-4-fluorophenyl)-7-((1-(4-ethylpiperazin-1-yl)-1-oxopropan-2-yl)oxy)isoquinolin-1(2H)-one, Example 177, or a pharmaceutically acceptable salt thereof:

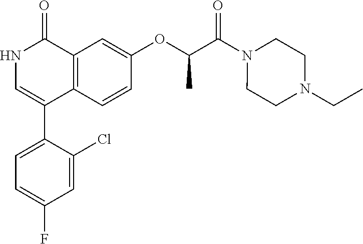 [SMILES: CCN1CCN(C(=O)[C@@H](C)Oc2ccc3c(-c4ccc(F)cc4Cl)c[nH]c(=O)c3c2)CC1]

In certain embodiments, the compound is (R)-4-(2-chloro-4-fluorophenyl)-7-((1-(4-(2-methoxyethyl)piperazin-1-yl)-1-oxopropan-2-yl)oxy)isoquinolin-1(2H)-one, Example 178, or a pharmaceutically acceptable salt thereof:

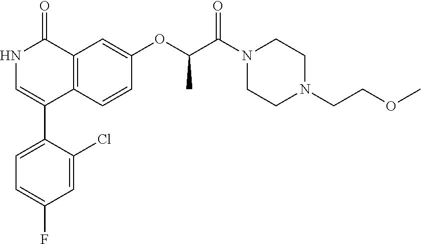 [SMILES: COCCN1CCN(C(=O)[C@@H](C)Oc2ccc3c(-c4ccc(F)cc4Cl)c[nH]c(=O)c3c2)CC1]

In certain embodiments, the compound is (R)-7-((1-(4-(2-methoxyethyl)piperazin-1-yl)-1-oxopropan-2-yl)oxy)-4-(o-tolyl)isoquinolin-1(2H)-one, Example 179, or a pharmaceutically acceptable salt thereof:

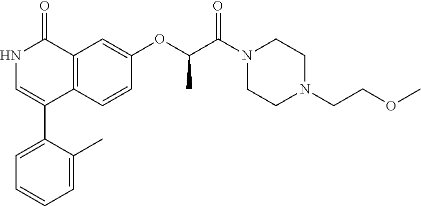 [SMILES: COCCN1CCN(C(=O)[C@@H](C)Oc2ccc3c(-c4ccccc4C)c[nH]c(=O)c3c2)CC1]

In certain embodiments, the compound is (R)-7-((1-(4-acetylpiperazin-1-yl)-1-oxopropan-2-yl)oxy)-4-(o-tolyl)isoquinolin-1(2H)-one, Example 180, or a pharmaceutically acceptable salt thereof:

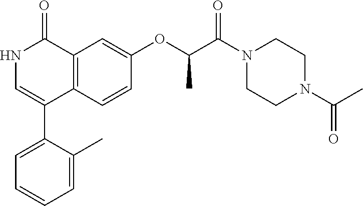 [SMILES: CC(=O)N1CCN(C(=O)[C@@H](C)Oc2ccc3c(-c4ccccc4C)c[nH]c(=O)c3c2)CC1]

In certain embodiments, the compound is (R)—N-(1-(2-((1-oxo-4-(o-tolyl)-1,2-dihydroisoquinolin-7-yl)oxy)propanoyl)piperidin-4-yl)acetamide, Example 181, or a pharmaceutically acceptable salt thereof:

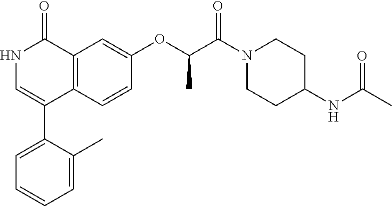 [SMILES: CC(=O)NC1CCN(C(=O)[C@@H](C)Oc2ccc3c(-c4ccccc4C)c[nH]c(=O)c3c2)CC1]

In certain embodiments, the compound is (R)—N-(1-(2-((4-(2-chloro-4-fluorophenyl)-1-oxo-1,2-dihydroisoquinolin-7-yl)oxy)propanoyl)piperidin-4-yl)acetamide, Example 182, or a pharmaceutically acceptable salt thereof:

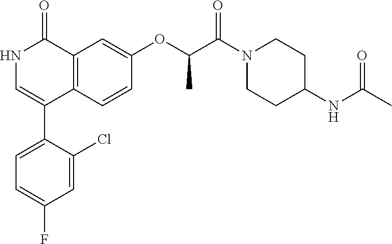 [SMILES: CC(=O)NC1CCN(C(=O)[C@@H](C)Oc2ccc3c(-c4ccc(F)cc4Cl)c[nH]c(=O)c3c2)CC1]

In certain embodiments, the compound is N—((R)-1-((R)-2-((1-oxo-4-(o-tolyl)-1,2-dihydroisoquinolin-7-yl)oxy)propanoyl)pyrrolidin-3-yl)acetamide, Example 183, or a pharmaceutically acceptable salt thereof:

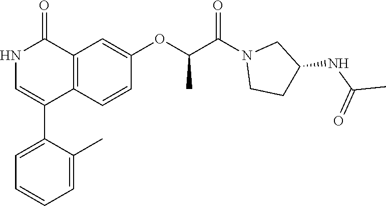 [SMILES: CC(=O)N[C@@H]1CCN(C(=O)[C@@H](C)Oc2ccc3c(-c4ccccc4C)c[nH]c(=O)c3c2)C1]

In certain embodiments, the compound is N—((S)-1-((R)-2-((1-oxo-4-(o-tolyl)-1,2-dihydroisoquinolin-7-yl)oxy)propanoyl)pyrrolidin-3-yl)acetamide, Example 184, or a pharmaceutically acceptable salt thereof:

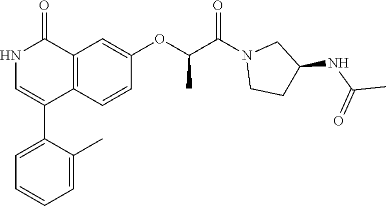 [SMILES: CC(=O)N[C@H]1CCN(C(=O)[C@@H](C)Oc2ccc3c(-c4ccccc4C)c[nH]c(=O)c3c2)C1]

In certain embodiments, the compound is (S)-4-((R)-2-((1-oxo-4-(o-tolyl)-1,2-dihydroisoquinolin-7-yl)oxy)propanoyl)morpholine-3-carboxamide, Example 185, or a pharmaceutically acceptable salt thereof:

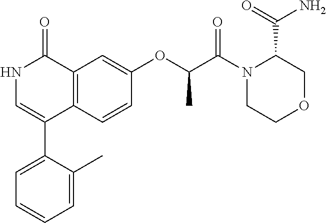 [SMILES: Cc1ccccc1-c1c[nH]c(=O)c2cc(O[C@H](C)C(=O)N3CCOC[C@H]3C(N)=O)ccc12]

In certain embodiments, the compound is (R)-4-((R)-2-((1-oxo-4-(o-tolyl)-1,2-dihydroisoquinolin-7-yl)oxy)propanoyl)morpholine-3-carboxamide, Example 186, or a pharmaceutically acceptable salt thereof:

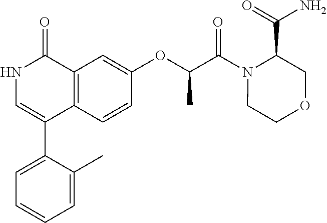 [SMILES: Cc1ccccc1-c1c[nH]c(=O)c2cc(O[C@H](C)C(=O)N3CCOC[C@@H]3C(N)=O)ccc12]

In certain embodiments, the compound is (R)-4-((R)-2-((1-oxo-4-(o-tolyl)-1,2-dihydroisoquinolin-7-yl)oxy)propanoyl)morpholine-3-carboxylic acid, Example 187, or a pharmaceutically acceptable salt thereof:

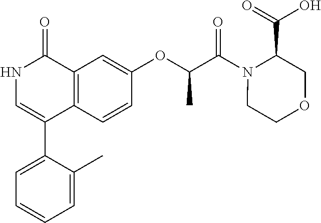 [SMILES: Cc1ccccc1-c1c[nH]c(=O)c2cc(O[C@H](C)C(=O)N3CCOC[C@@H]3C(=O)O)ccc12]

In certain embodiments, the compound is (S)-4-((R)-2-((1-oxo-4-(o-tolyl)-1,2-dihydroisoquinolin-7-yl)oxy)propanoyl)morpholine-3-carboxylic acid, Example 188, or a pharmaceutically acceptable salt thereof:

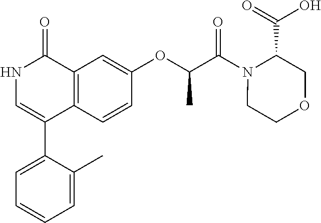 [SMILES: Cc1ccccc1-c1c[nH]c(=O)c2cc(O[C@H](C)C(=O)N3CCOC[C@H]3C(=O)O)ccc12]

In certain embodiments, the compound is (R)-4-((R)-2-((1-oxo-4-(o-tolyl)-1,2-dihydroisoquinolin-7-yl)oxy)propanoyl)morpholine-2-carboxamide, Example 189, or a pharmaceutically acceptable salt thereof:

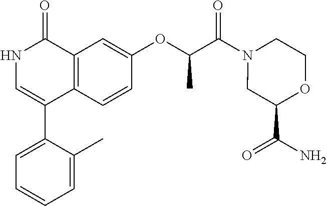 [SMILES: Cc1ccccc1-c1c[nH]c(=O)c2cc(O[C@H](C)C(=O)N3CCO[C@@H](C(N)=O)C3)ccc12]

In certain embodiments, the compound is (S)-4-((R)-2-((1-oxo-4-(o-tolyl)-1,2-dihydroisoquinolin-7-yl)oxy)propanoyl)morpholine-2-carboxamide, Example 190, or a pharmaceutically acceptable salt thereof:

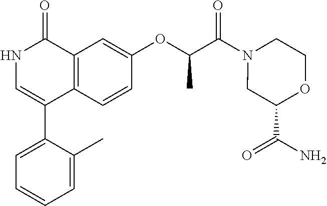 [SMILES: Cc1ccccc1-c1c[nH]c(=O)c2cc(O[C@H](C)C(=O)N3CCO[C@H](C(N)=O)C3)ccc12]

In certain embodiments, the compound is (S)-4-((R)-2-((1-oxo-4-(o-tolyl)-1,2-dihydroisoquinolin-7-yl)oxy)propanoyl)morpholine-2-carboxylic acid, Example 191, or a pharmaceutically acceptable salt thereof:

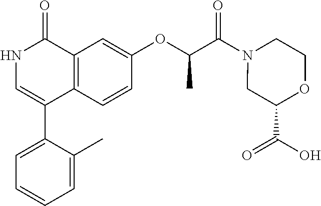 [SMILES: Cc1ccccc1-c1c[nH]c(=O)c2cc(O[C@H](C)C(=O)N3CCO[C@H](C(=O)O)C3)ccc12]

In certain embodiments, the compound is (R)-4-((R)-2-((1-oxo-4-(o-tolyl)-1,2-dihydroisoquinolin-7-yl)oxy)propanoyl)morpholine-2-carboxylic acid, Example 192, or a pharmaceutically acceptable salt thereof:

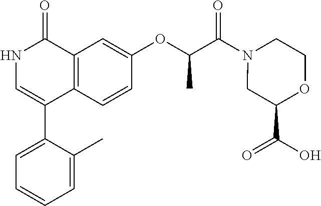 [SMILES: Cc1ccccc1-c1c[nH]c(=O)c2cc(O[C@H](C)C(=O)N3CCO[C@@H](C(=O)O)C3)ccc12]

In certain embodiments, the compound is (S)-4-((R)-2-((4-(2,6-dimethylphenyl)-1-oxo-1,2-dihydroisoquinolin-7-yl)oxy)propanoyl)morpholine-3-carboxamide, Example 193, or a pharmaceutically acceptable salt thereof:

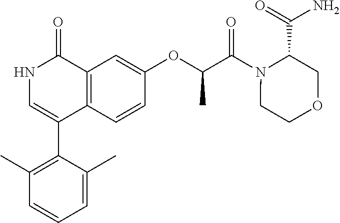 [SMILES: Cc1cccc(C)c1-c1c[nH]c(=O)c2cc(O[C@H](C)C(=O)N3CCOC[C@H]3C(N)=O)ccc12]

In certain embodiments, the compound is (R)-4-((R)-2-((4-(2,6-dimethylphenyl)-1-oxo-1,2-dihydroisoquinolin-7-yl)oxy)propanoyl)morpholine-3-carboxamide, Example 194, or a pharmaceutically acceptable salt thereof:

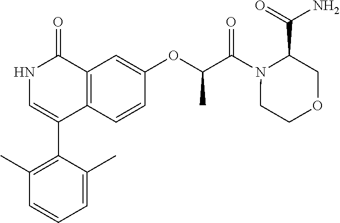 [SMILES: Cc1cccc(C)c1-c1c[nH]c(=O)c2cc(O[C@H](C)C(=O)N3CCOC[C@@H]3C(N)=O)ccc12]

In certain embodiments, the compound is (R)-1-((R)-2-((4-(2-chloro-4-fluorophenyl)-1-oxo-1,2-dihydroisoquinolin-7-yl)oxy)propanoyl)-5,5-difluoropiperidine-3-carboxamide, Example 195, or a pharmaceutically acceptable salt thereof:

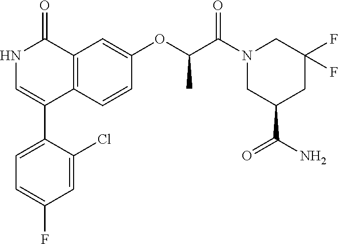 [SMILES: C[C@@H](Oc1ccc2c(-c3ccc(F)cc3Cl)c[nH]c(=O)c2c1)C(=O)N1C[C@H](C(N)=O)CC(F)(F)C1]

In certain embodiments, the compound is (R)-5,5-difluoro-1-((R)-2-((1-oxo-4-(o-tolyl)-1,2-dihydroisoquinolin-7-yl)oxy)propanoyl)piperidine-3-carboxamide, Example 196, or a pharmaceutically acceptable salt thereof:

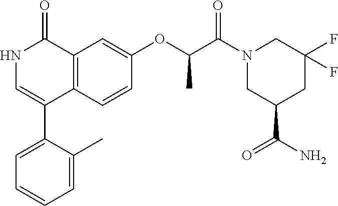 [SMILES: Cc1ccccc1-c1c[nH]c(=O)c2cc(O[C@H](C)C(=O)N3C[C@H](C(N)=O)CC(F)(F)C3)ccc12]

In certain embodiments, the compound is (S)-5,5-difluoro-1-((R)-2-((1-oxo-4-(o-tolyl)-1,2-dihydroisoquinolin-7-yl)oxy)propanoyl)piperidine-3-carboxamide, Example 197, or a pharmaceutically acceptable salt thereof:

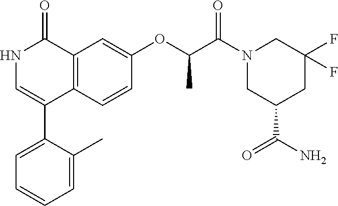 [SMILES: Cc1ccccc1-c1c[nH]c(=O)c2cc(O[C@H](C)C(=O)N3C[C@@H](C(N)=O)CC(F)(F)C3)ccc12]

In certain embodiments, the compound is (S)-5,5-difluoro-1-((R)-2-((4-(2-(methyl-d₃)phenyl)-1-oxo-1,2-dihydroisoquinolin-7-yl)oxy)propanoyl)piperidine-3-carboxamide, Example 198, or a pharmaceutically acceptable salt thereof:

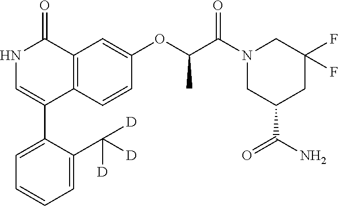 [SMILES: [2H]C([2H])([2H])c1ccccc1-c1c[nH]c(=O)c2cc(O[C@H](C)C(=O)N3C[C@@H](C(N)=O)CC(F)(F)C3)ccc12]

In certain embodiments, the compound is (R)-5,5-difluoro-1-((R)-2-((4-(2-(methyl-d₃)phenyl)-1-oxo-1,2-dihydroisoquinolin-7-yl)oxy)propanoyl)piperidine-3-carboxamide, Example 199, or a pharmaceutically acceptable salt thereof:

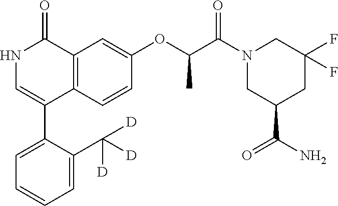 [SMILES: [2H]C([2H])([2H])c1ccccc1-c1c[nH]c(=O)c2cc(O[C@H](C)C(=O)N3C[C@H](C(N)=O)CC(F)(F)C3)ccc12]

In certain embodiments, the compound is 7-(2,2-difluoroethoxy)-4-(o-tolyl)isoquinolin-1(2H)-one, Example 200, or a pharmaceutically acceptable salt thereof:

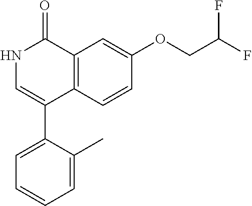 [SMILES: Cc1ccccc1-c1c[nH]c(=O)c2cc(OCC(F)F)ccc12]

In certain embodiments, the compound is 4-(2-(methyl-d₃)phenyl)-7-(2,2,2-trifluoroethoxy)isoquinolin-1(2H)-one, Example 201, or a pharmaceutically acceptable salt thereof:

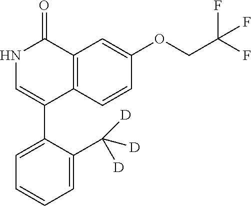 [SMILES: [2H]C([2H])([2H])c1ccccc1-c1c[nH]c(=O)c2cc(OCC(F)(F)F)ccc12]

In certain embodiments, the compound is 7-(2,2-difluoroethoxy)-4-(2-(methyl-d₃)phenyl)isoquinolin-1(2H)-one, Example 202, or a pharmaceutically acceptable salt thereof:

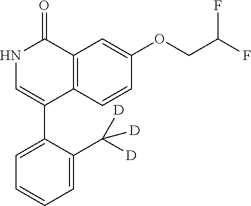 [SMILES: [2H]C([2H])([2H])c1ccccc1-c1c[nH]c(=O)c2cc(OCC(F)F)ccc12]

In certain embodiments, the compound is 7-(((R)-1-((S)-3-(hydroxymethyl)morpholino)-1-oxopropan-2-yl)oxy)-4-(o-tolyl)isoquinolin-1(2H)-one, Example 203, or a pharmaceutically acceptable salt thereof:

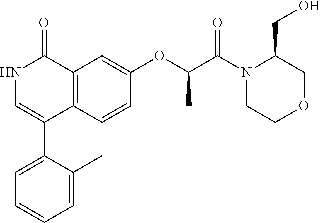 [SMILES: Cc1ccccc1-c1c[nH]c(=O)c2cc(O[C@H](C)C(=O)N3CCOC[C@@H]3CO)ccc12]

In certain embodiments, the compound is 4-(4-fluoro-2-methylphenyl)-7-(((R)-1-((S)-3-(hydroxymethyl)morpholino)-1-oxopropan-2-yl)oxy)isoquinolin-1(2H)-one, Example 204, or a pharmaceutically acceptable salt thereof:

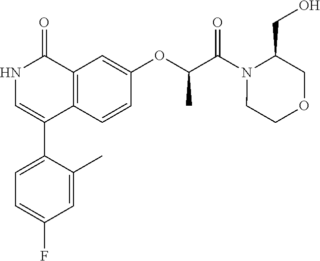 [SMILES: Cc1cc(F)ccc1-c1c[nH]c(=O)c2cc(O[C@H](C)C(=O)N3CCOC[C@@H]3CO)ccc12]

In certain embodiments, the compound is 4-(5-fluoro-2-methylphenyl)-7-(((R)-1-((S)-3-(hydroxymethyl)morpholino)-1-oxopropan-2-yl)oxy)isoquinolin-1(2H)-one, Example 205, or a pharmaceutically acceptable salt thereof:

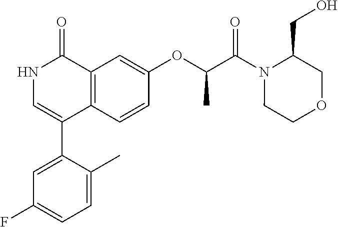 [SMILES: Cc1ccc(F)cc1-c1c[nH]c(=O)c2cc(O[C@H](C)C(=O)N3CCOC[C@@H]3CO)ccc12]

In certain embodiments, the compound is 4-(3-fluoro-2-methylphenyl)-7-(((R)-1-((S)-3-(hydroxymethyl)morpholino)-1-oxopropan-2-yl)oxy)isoquinolin-1(2H)-one, Example 206, or a pharmaceutically acceptable salt thereof:

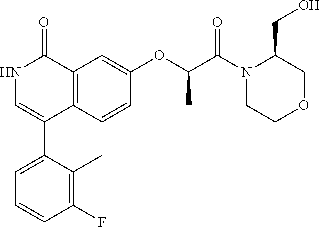 [SMILES: Cc1c(F)cccc1-c1c[nH]c(=O)c2cc(O[C@H](C)C(=O)N3CCOC[C@@H]3CO)ccc12]

In certain embodiments, the compound is 7-(((R)-1-((S)-3-(hydroxymethyl)morpholino)-1-oxopropan-2-yl)oxy)-4-(2-(methyl-d₃)phenyl)isoquinolin-1(2H)-one, Example 207, or a pharmaceutically acceptable salt thereof:

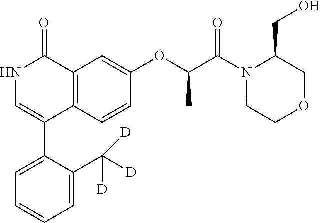 [SMILES: [2H]C([2H])([2H])c1ccccc1-c1c[nH]c(=O)c2cc(O[C@H](C)C(=O)N3CCOC[C@@H]3CO)ccc12]

In certain embodiments, the compound is (R)-4-((R)-2-((4-(2-(methyl-d₃)phenyl)-1-oxo-1,2-dihydroisoquinolin-7-yl)oxy)propanoyl)morpholine-3-carboxamide, Example 208, or a pharmaceutically acceptable salt thereof:

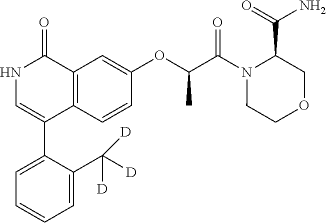 [SMILES: [2H]C([2H])([2H])c1ccccc1-c1c[nH]c(=O)c2cc(O[C@H](C)C(=O)N3CCOC[C@@H]3C(N)=O)ccc12]

In certain embodiments, the compound is (S)-4-((R)-2-((4-(2-(methyl-d₃)phenyl)-1-oxo-1,2-dihydroisoquinolin-7-yl)oxy)propanoyl)morpholine-3-carboxamide, Example 209, or a pharmaceutically acceptable salt thereof:

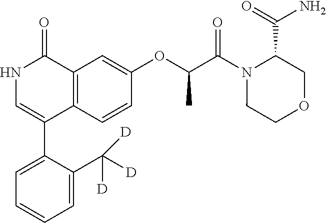 [SMILES: [2H]C([2H])([2H])c1ccccc1-c1c[nH]c(=O)c2cc(O[C@H](C)C(=O)N3CCOC[C@H]3C(N)=O)ccc12]

In certain embodiments, the compound is 7-(2,2-difluoroethoxy)-4-(4-fluoro-2-methylphenyl)isoquinolin-1(2H)-one, Example 210, or a pharmaceutically acceptable salt thereof:

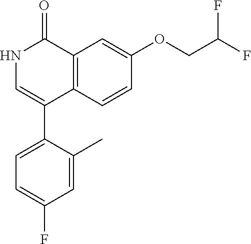 [SMILES: Cc1cc(F)ccc1-c1c[nH]c(=O)c2cc(OCC(F)F)ccc12]

In certain embodiments, the compound is 7-(2,2-difluoroethoxy)-4-(5-fluoro-2-methylphenyl)isoquinolin-1(2H)-one, Example 211, or a pharmaceutically acceptable salt thereof:

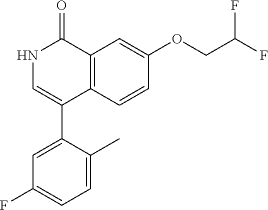 [SMILES: Cc1ccc(F)cc1-c1c[nH]c(=O)c2cc(OCC(F)F)ccc12]

In certain embodiments, the compound is 7-(2,2-difluoroethoxy)-4-(3-fluoro-2-methylphenyl)isoquinolin-1(2H)-one, Example 212, or a pharmaceutically acceptable salt thereof:

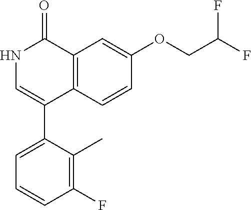 [SMILES: Cc1c(F)cccc1-c1c[nH]c(=O)c2cc(OCC(F)F)ccc12]

In certain embodiments, the compound is 4-(5-fluoro-2-methylphenyl)-7-(2,2,2-trifluoroethoxy)isoquinolin-1(2H)-one, Example 213, or a pharmaceutically acceptable salt thereof:

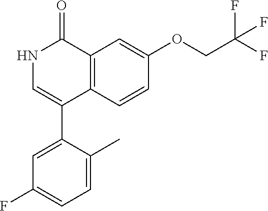 [SMILES: Cc1ccc(F)cc1-c1c[nH]c(=O)c2cc(OCC(F)(F)F)ccc12]

In certain embodiments, the compound is 4-(4-fluoro-2-methylphenyl)-7-(2,2,2-trifluoroethoxy)isoquinolin-1(2H)-one, Example 214, or a pharmaceutically acceptable salt thereof:

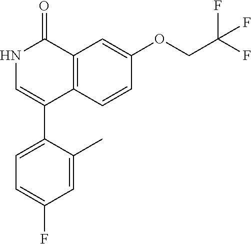 [SMILES: Cc1cc(F)ccc1-c1c[nH]c(=O)c2cc(OCC(F)(F)F)ccc12]

In certain embodiments, the compound is 4-(3-fluoro-2-methylphenyl)-7-(2,2,2-trifluoroethoxy)isoquinolin-1(2H)-one, Example 215, or a pharmaceutically acceptable salt thereof:

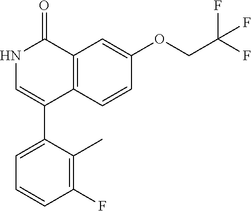 [SMILES: Cc1c(F)cccc1-c1c[nH]c(=O)c2cc(OCC(F)(F)F)ccc12]

In certain embodiments, the compound is 2-((4-(2-methoxyphenyl)-1-oxo-1,2-dihydroisoquinolin-7-yl)oxy)acetonitrile, Example 216, or a pharmaceutically acceptable salt thereof:

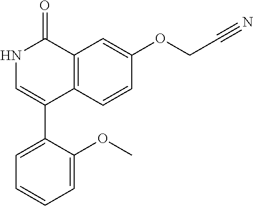 [SMILES: COc1ccccc1-c1c[nH]c(=O)c2cc(OCC#N)ccc12]

In certain embodiments, the compound is 4-(o-tolyl)-7-((trifluoromethoxy)methyl)isoquinolin-1(2H)-one, Example 217, or a pharmaceutically acceptable salt thereof:

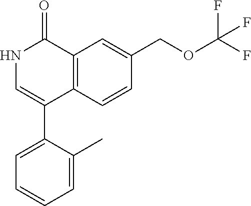 [SMILES: Cc1ccccc1-c1c[nH]c(=O)c2cc(COC(F)(F)F)ccc12]

In certain embodiments, the compound is 7-(4-fluorophenoxy)-4-(o-tolyl)isoquinolin-1(2H)-one, Example 218, or a pharmaceutically acceptable salt thereof:

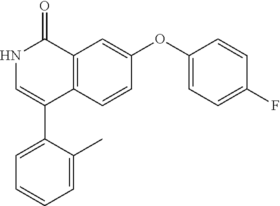 [SMILES: Cc1ccccc1-c1c[nH]c(=O)c2cc(Oc3ccc(F)cc3)ccc12]

In certain embodiments, the compound is 7-(3-fluorophenethoxy)-4-(o-tolyl)isoquinolin-1(2H)-one, Example 219, or a pharmaceutically acceptable salt thereof:

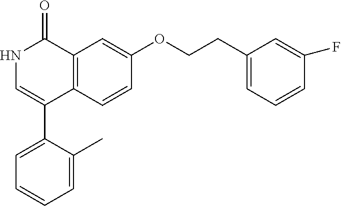 [SMILES: Cc1ccccc1-c1c[nH]c(=O)c2cc(OCCc3cccc(F)c3)ccc12]

In certain embodiments, the compound is 4-(o-tolyl)-7-(2-(2,2,2-trifluoroethoxy)ethoxy)isoquinolin-1(2H)-one, Example 220, or a pharmaceutically acceptable salt thereof:

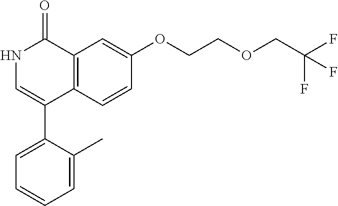 [SMILES: Cc1ccccc1-c1c[nH]c(=O)c2cc(OCCOCC(F)(F)F)ccc12]

In certain embodiments, the compound is 4-(2-(methyl-d₃)phenyl)-7-((trifluoromethoxy)methyl)isoquinolin-1(2H)-one, Example 221, or a pharmaceutically acceptable salt thereof:

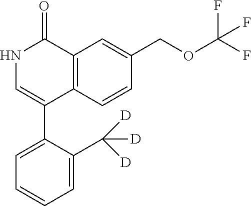 [SMILES: [2H]C([2H])([2H])c1ccccc1-c1c[nH]c(=O)c2cc(COC(F)(F)F)ccc12]

In certain embodiments, the compound is 4-(2-(methyl-d₃)phenyl)-7-((2,2,2-trifluoroethoxy)methyl)isoquinolin-1(2H)-one, Example 222, or a pharmaceutically acceptable salt thereof:

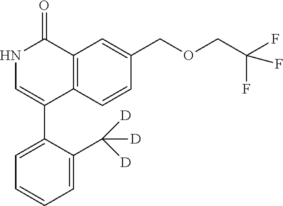 [SMILES: [2H]C([2H])([2H])c1ccccc1-c1c[nH]c(=O)c2cc(COCC(F)(F)F)ccc12]

In certain embodiments, the compound is 4-(o-tolyl)-7-((2,2,2-trifluoroethoxy)methyl)isoquinolin-1(2H)-one, Example 223, or a pharmaceutically acceptable salt thereof:

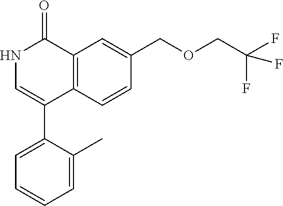 [SMILES: Cc1ccccc1-c1c[nH]c(=O)c2cc(COCC(F)(F)F)ccc12]

In another embodiment, the present invention is directed to a compound, a prodrug thereof, or a pharmaceutically acceptable salt thereof, represented by formula (II):

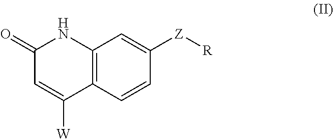

wherein
Z is O;
W is C₆-C₁₂ aryl or 5- to 12-membered heteroaryl, either of which is substituted with one or more groups, each independently selected from the group consisting of fluoro, chloro, C₁-C₄ alkyl, trifluoromethyl, difluoromethyl, cyano, hydroxyl, and C₁-C₄ alkoxyl, provided that at least one substituent is at an ortho position relative to the attachment point with the central ring;
R is hydrogen, or C₁-C₆ alkyl optionally substituted with one or more groups each independently selected from the group consisting of fluoro, hydroxyl, C₁-C₄ alkoxyl, C₁-C₄ haloalkoxyl, carboxyl, C(O)NR⁵R⁶, NR⁵R⁶, and NR²R³,
or R is C₆-C₁₂ aryl optionally substituted with one or more groups, each independently selected from the group consisting of fluoro, chloro, C₁-C₄ alkyl, trifluoromethyl, difluoromethyl, cyano, hydroxyl, NR⁵R⁶, and C₁-C₄ alkoxyl,
or R is C₁-C₄ haloalkoxyl, CH₂CH₂R⁸, CR³R⁴C(O)OR⁵, or CR³R⁴C(O)NR⁵R⁶;
each R¹ is independently hydrogen, C₁-C₄ alkoxyl, or C₁-C₄ alkyl optionally substituted with one or more fluoro groups;
each R² is independently hydrogen, C₁-C₄ alkyl optionally substituted with one or more fluoro groups, C(O)C₁-C₄ alkyl optionally substituted with one or more fluoro groups, or C(O)NHC₁-C₄ alkyl optionally substituted with one or more fluoro groups;
R³ and R⁴ are each independently hydrogen or C₁-C₄ alkyl optionally substituted with one or more groups each independently selected from the group consisting of fluoro, hydroxyl, C₁-C₄ alkoxyl, C₁-C₄ haloalkoxyl, and NR⁵R⁶;
R⁵ and R⁶ are each independently hydrogen or C₁-C₄ alkyl optionally substituted with one or more groups each independently selected from the group consisting of fluoro, hydroxyl, cyano, C₁-C₄ alkoxyl, C₁-C₄ haloalkoxyl, and NR¹R²;
or if R⁵ and R⁶ are attached to the same nitrogen atom, R⁵ and R⁶ together with their connecting nitrogen form a 5- or 6-membered heterocyclic ring optionally containing another heteroatom that is N, O, or S, and such 5- or 6-membered heterocyclic ring is optionally substituted with one or two groups each independently selected from the group consisting of fluoro, chloro, C₁-C₄ alkyl, carboxyl, and C₁-C₄ alkylcarboxylate; and
R⁸ is aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R⁸ is OR¹, NR¹R², C(O)R¹, or C(O)NR²R².
   For the compounds of Formula (II), in certain embodiments, Z is O.

In certain embodiments, W is C₆ aryl.

In certain embodiments, W is C₆ aryl substituted with one or more groups, each independently selected from the group consisting of fluoro, chloro, and C₁ alkyl.

In certain embodiments, W is 2-chloro-4-fluorophenyl.

In certain embodiments, W is 2-methyl-4-fluorophenyl.

In certain embodiments, W is 2-methylphenyl.

In certain embodiments, R is hydrogen.

In certain embodiments, R is C₁-C₄ alkyl optionally substituted with C(O)OH and C(O)NR⁵R⁶.

In certain embodiments, R is methyl.

In certain embodiments, R is isopropyl.

In certain embodiments, R is sec-butyl.

In certain embodiments, R is isobutyl.

In certain embodiments, R⁵ and R⁶ are each independently C₁ alkyl.

In certain embodiments, R⁵ and R⁶ are attached to the same nitrogen atom and together with their connecting nitrogen form a 6-membered heterocyclic ring that is optionally substituted with a carboxyl group.

In certain embodiments, W is phenyl substituted at the ortho position relative to the attachment point of the quinolinone ring.

In certain embodiments, the compound is 4-(2-chloro-4-fluorophenyl)-7-methoxyquinolin-2(1H)-one, Example Q1, or a pharmaceutically acceptable salt thereof:

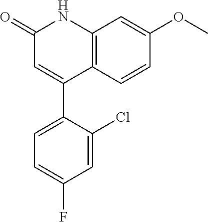 [SMILES: COc1ccc2c(-c3ccc(F)cc3Cl)cc(=O)[nH]c2c1]

In certain embodiments, the compound is 4-(2-chloro-4-fluorophenyl)-7-hydroxyquinolin-2(1H)-one, Example Q2, or a pharmaceutically acceptable salt thereof:

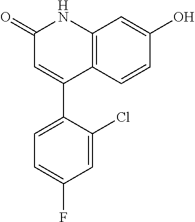 [SMILES: O=c1cc(-c2ccc(F)cc2Cl)c2ccc(O)cc2[nH]1]

In certain embodiments, the compound is 4-(4-fluoro-2-methylphenyl)-7-isopropoxy-1H-quinolin-2-one, Example Q3, or a pharmaceutically acceptable salt thereof:

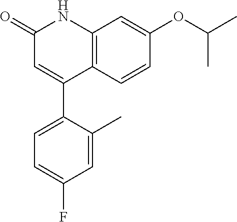 [SMILES: Cc1cc(F)ccc1-c1cc(=O)[nH]c2cc(OC(C)C)ccc12]

In certain embodiments, the compound is 4-(2-chloro-4-fluorophenyl)-7-isopropoxyquinolin-2(1H)-one, Example Q4, or a pharmaceutically acceptable salt thereof:

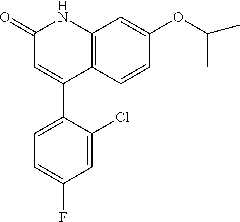 [SMILES: CC(C)Oc1ccc2c(-c3ccc(F)cc3Cl)cc(=O)[nH]c2c1]

In certain embodiments, the compound is (R)-4-(2-chloro-4-fluorophenyl)-7-((1-oxo-1-(piperidin-1-yl)propan-2-yl)oxy)quinolin-2(1H)-one, Example Q5, or a pharmaceutically acceptable salt thereof:

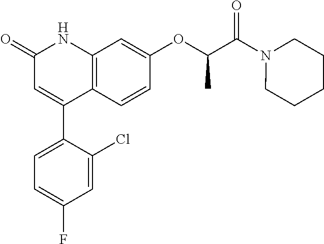 [SMILES: C[C@@H](Oc1ccc2c(-c3ccc(F)cc3Cl)cc(=O)[nH]c2c1)C(=O)N1CCCCC1]

In certain embodiments, the compound is (R)-2-((4-(2-chloro-4-fluorophenyl)-2-oxo-1,2-dihydroquinolin-7-yl)oxy)-N,N-dimethylpropanamide, Example Q6, or a pharmaceutically acceptable salt thereof:

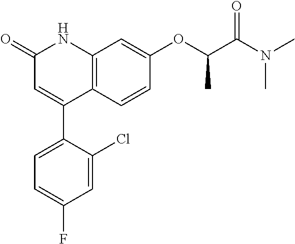 [SMILES: C[C@@H](Oc1ccc2c(-c3ccc(F)cc3Cl)cc(=O)[nH]c2c1)C(=O)N(C)C]

In certain embodiments, the compound is (R)—N,N-dimethyl-2-((2-oxo-4-(o-tolyl)-1,2-dihydroquinolin-7-yl)oxy)propanamide, Example Q7, or a pharmaceutically acceptable salt thereof:

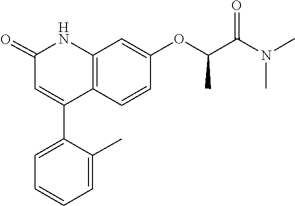 [SMILES: Cc1ccccc1-c1cc(=O)[nH]c2cc(O[C@H](C)C(=O)N(C)C)ccc12]

In certain embodiments, the compound is (S)—N,N-dimethyl-2-((2-oxo-4-(o-tolyl)-1,2-dihydroquinolin-7-yl)oxy)propanamide, Example Q8, or a pharmaceutically acceptable salt thereof:

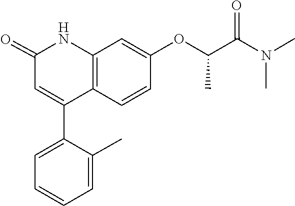 [SMILES: Cc1ccccc1-c1cc(=O)[nH]c2cc(O[C@@H](C)C(=O)N(C)C)ccc12]

In certain embodiments, the compound is 7-methoxy-4-phenylquinolin-2(1H)-one, Example Q9, or a pharmaceutically acceptable salt thereof:

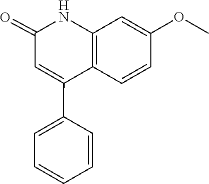 [SMILES: COc1ccc2c(-c3ccccc3)cc(=O)[nH]c2c1]

In certain embodiments, the compound is 7-isopropoxy-4-(o-tolyl)quinolin-2(11)-one, Example Q10, or a pharmaceutically acceptable salt thereof:

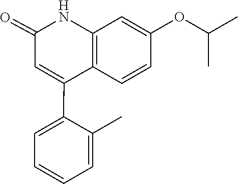 [SMILES: Cc1ccccc1-c1cc(=O)[nH]c2cc(OC(C)C)ccc12]

In certain embodiments, the compound is 7-(sec-butoxy)-4-(o-tolyl)quinolin-2(11)-one, Example Q11, or a pharmaceutically acceptable salt thereof:

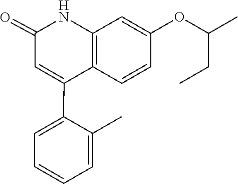 [SMILES: CCC(C)Oc1ccc2c(-c3ccccc3C)cc(=O)[nH]c2c1]

In certain embodiments, the compound is 7-isobutoxy-4-(o-tolyl)quinolin-2(11)-one, Example Q12, or a pharmaceutically acceptable salt thereof:

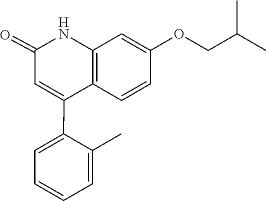 [SMILES: Cc1ccccc1-c1cc(=O)[nH]c2cc(OCC(C)C)ccc12]

In certain embodiments, the compound is (R)-2-((2-oxo-4-(o-tolyl)-1,2-dihydroquinolin-7-yl)oxy)propanoic acid, Example Q13, or a pharmaceutically acceptable salt thereof:

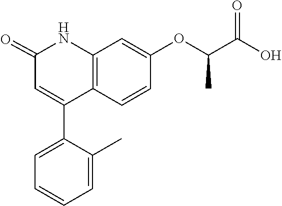 [SMILES: Cc1ccccc1-c1cc(=O)[nH]c2cc(O[C@H](C)C(=O)O)ccc12]

In certain embodiments, the compound is (R)-1-(2-((2-oxo-4-(o-tolyl)-1,2-dihydroquinolin-7-yl)oxy)propanoyl)piperidine-4-carboxylic acid, Example Q14, or a pharmaceutically acceptable salt thereof:

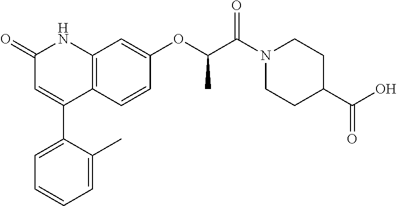 [SMILES: Cc1ccccc1-c1cc(=O)[nH]c2cc(O[C@H](C)C(=O)N3CCC(C(=O)O)CC3)ccc12]

In certain embodiments, the compound is (S)-1-((R)-2-((2-oxo-4-(o-tolyl)-1,2-dihydroquinolin-7-yl)oxy)propanoyl)piperidine-3-carboxylic acid, Example Q15, or a pharmaceutically acceptable salt thereof:

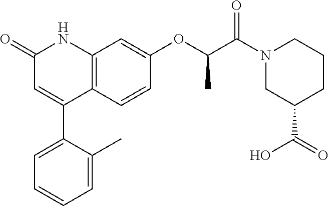 [SMILES: Cc1ccccc1-c1cc(=O)[nH]c2cc(O[C@H](C)C(=O)N3CCC[C@H](C(=O)O)C3)ccc12]

In certain embodiments, the compounds inhibits POLRMT.

In certain embodiments, the compounds promote POLRMT.

The compounds of the present invention may contain asymmetric carbon atoms (sometimes as the result of a deuterium atom) and thereby can exist as either individual stereoisomers or mixtures of the enantiomers or mixtures of diastereomers. Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. Carbon atoms labelled with * or ** refer to a compound that is chiral but the absolute stereochemistry has not been determined.

The compounds of the present invention may contain double bonds that may exist in more than one geometric isomer. Examples of such double bonds are carbon-carbon double bonds which form alkenes. In the case of carbon-carbon double bonds, the geometric isomers may be E or Z isomers.

Unless otherwise indicated, when a disclosed compound is named or depicted by a structure without specifying the geometric isomerism and has one or more possible geometric isomers, it is understood to represent all possible geometric isomers of the compound.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and ketoenol tautomerism is possible, it is understood that the substituent might in fact be present, in whole or in part, in the oxo (═O) form.

Compounds of the present invention may exist in amorphous form and/or one or more crystalline forms. As such all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the invention. See, e.g., Gannes, L Z et al., Comp Biochem Physiol Mol Integr Physiol, 119, 725 (1998). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium. See Pirali et al, J. Med. Chem. 62, 5276-97 (2019). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances deuteration may have little to no impact on these properties, or may affect them in an undesirable manner. Whether and/or how such replacement will impact drug properties can only be determined if the drug is synthesized, evaluated, and compared to its non-deuterated counterpart. See Fukuto et al., J. Med. Chem. 34, 2871-76 (1991). Because some drugs have multiple sites of metabolism or more than one active sites for binding to a target, it is unpredictable as to which sites may benefit by deuterium replacement or to what extent isotope enrichment is necessary to produce a beneficial effect.

### Preparation of the Compounds

The starting materials and reagents used in each step in the preparation are known and can be readily prepared or purchased from commercial sources.

The compound obtained in each step can also be used for the next reaction as a reaction mixture thereof or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min. to 48 h, preferably 10 min. to 8 h.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water.

Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schutzenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 4^{th} Ed.” (Theodora W. Greene, Peter G. M. Wuts) Wiley-Interscience, 2007; “Protecting Groups 3rd Ed.” (P. J. Kocienski) Thieme, 2004 and the like.

Deuterated POLRMT modulators of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (“NMR”) can be used to determine a compound's structure while mass spectroscopy (“MS”) can be used to determine the amount of deuterium atom in the compound by comparison to its non-deuterated form.

### Compositions

The present invention further includes pharmaceutical compositions of the compounds, a pharmaceutically acceptable salt of said compounds, or prodrugs of said compounds. In addition to the compound of the invention, a salt thereof, or a prodrug thereof, the pharmaceutical compositions comprise one or more pharmaceutically acceptable excipients, such excipients being compatible with other ingredients in the composition and also being not toxic or otherwise harmful. Examples of excipients include carriers, lubricants, binders, disintegrants, solvents, solubilizing agents, suspending agents, isotonic agents, buffers, soothing agents, preservatives, antioxidants, colorants, taste-modifying agents, absorbents, and/or wetting agents.

The pharmaceutical compositions of the invention include those suitable for oral, rectal, nasal, topical, buccal, sublingual, vaginal or parenteral (including subcutaneous, intramuscular, intravenous and intradermal) administration. Such compositions may be prepared by any methods well known in the art of pharmaceutical formulations and pharmacy. See, e.g., Remington: The Science and Practice of Pharmacy, Elsevier Science, 23rd ed. (2020).

Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, hydrogels, gelatin, hydrogenated naphthalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions can be administered by oral ingestion. Compositions intended for oral use can be prepared in solid or liquid forms, according to any method known to a person of ordinary skill in the art for the manufacture of pharmaceutical compositions. Solid dosage forms for oral administration include capsules (both soft and hard gelatin capsules), tablets, powders, and granules. Generally, these pharmaceutical preparations contain active ingredients admixed with pharmaceutically acceptable excipients. These excipients include, for example, inert diluents, such as calcium carbonate, sodium carbonate, lactose, sucrose, glucose, mannitol, cellulose, starch, calcium phosphate, sodium phosphate, kaolin and the like; binding agents, buffering agents, and/or lubricating agents (e.g., magnesium stearate) may also be used. Tablets and capsules can additionally be prepared with release-controlling coatings such as enteric coatings. The compositions may optionally contain sweetening, flavoring, coloring, perfuming, and preserving agents in order to provide a more palatable preparation.

In another embodiment, a pharmaceutical composition of this invention further comprises a second therapeutic agent. The second therapeutic agent may be selected from any pharmaceutically active compound; preferably the second therapeutic agent is known to treat cancer, neurodegenerative disorders, or metabolic disorders. Alternatively, the compounds of the invention and second therapeutic agent may be administered together (within less than 24 hours of one another, consecutively or simultaneously) but in separate pharmaceutical compositions. In certain embodiments, the compounds on the invention and second therapeutic agent can be administered separately (e.g., more than 24 hours of one another.) If the second therapeutic agent acts synergistically with the compounds of this invention, the therapeutically effective amount of such compounds and/or the second therapeutic agent may be less that such amount required when either is administered alone.

For the treatment of cancer, the compounds described herein may be administered in combination with a chemotherapeutic agent. Therapeutically effective amounts of the additional chemotherapeutic agent(S) are well known to those skilled in the art. However, it is well within the attending physician to determine the amount of other chemotherapeutic agent(S) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting.

### EXAMPLES

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by mass spectrometry and/or NMR, where peaks assigned to the characteristic protons in the title compound are presented where appropriate. ¹H NMR shift (6) are given in parts per million (ppm) down field from an internal reference standard.

Table 1 and Table 2 provide a listing of exemplary compounds of the present invention and their IC₅₀ values for inhibition of POLRMT.

The abbreviations used herein are known to a person of ordinary skill in the art. A partial list of abbreviations that may be used herein include: acetonitrile (MeCN), ammonium carbonate (NH₄)₂CO₃, ammonium chloride (NH₄Cl), aqueous (aq.), 1,1′-bis(diphenylphosphino)ferrocene (dppf), 1,3-bis(diphenylphosphino)propane (dppp), bis(pinacolato)diboron (B₂pin₂), N-bromosuccinimide (NBS), bromo-tris-pyrrolidino-phosphonium hexafluorophosphate (PyBroP), boron tribromide (BBr₃), butyl lithium (BuLi), calculated (Calcd.), cesium carbonate (Cs₂CO₃), dichloromethane (DCM, CH₂Cl₂), N,N-dicyclohexylcarbodiimide (DCC), dichloroethane (DCE), diethyl azodicarboxylate (DEAD), diisopropyl azodicarboxylate (DIAD), N,N-diisopropylethylamine (DIPEA), 4-dimethylaminopyridine (DMAP), dimethylformamide (DMF), dimethyl sulfoxide (DMSO), di-tert-butyl decarbonate (Boc₂O), 1-ethyl-3-(3-dimethylaminopropyl)carbodiimide (EDC), electrospray ionization (ESI), enantiomeric excess (ee), ethyl acetate (EtOAc), hour (h.), N-[(dimethylamino)-1H-1,2,3-triazolo-[4,5-b]pyridin-1-ylmethylene]-N-methylmethanaminium hexafluorophosphate N-oxide (HATU), high performance liquid chromatography (HPLC), hydroxybenzotriazole (HOBt), isopropyl alcohol (IPA), lithium hydroxide monohydrate (LiOH·H2O), methanol (MeOH), methyl iodide (Mel), minutes (min.), potassium carbonate (K₂CO₃), liquid chromatography-mass spectrometry (LCMS), phenyliodide(III) diacetate (PIDA), propylphosphonic anhydride (T₃P), reverse phase (RP), room/ambient temperature (rt, RT), silver oxide (Ag₂O), sodium hydride (NaH), sodium sulfate (Na₂SO₃), supercritical fluid chromatography (SFC), tetrahydrofuran (THF), triethylamine (Et₃N), thionyl chloride (SOCl₂), triphenylphosphine (PPh₃), dicyclohexyl[2′,4′,6′-tris(propan-2-yl)[1,1′-biphenyl]-2-yl]phosphane (XPhos).

Table 1 and Table 2 provide a listing of exemplary compounds of the present invention and their IC₅₀ values for inhibition of POLRMT.

### Examples 1-2: Synthesis of 4-(2-chloro-4-fluorophenyl)-7-methoxyisoquinolin-1(2H)-one and 4-(2-chloro-4-fluorophenyl)-7-hydroxyisoquinolin-1(2H)-one

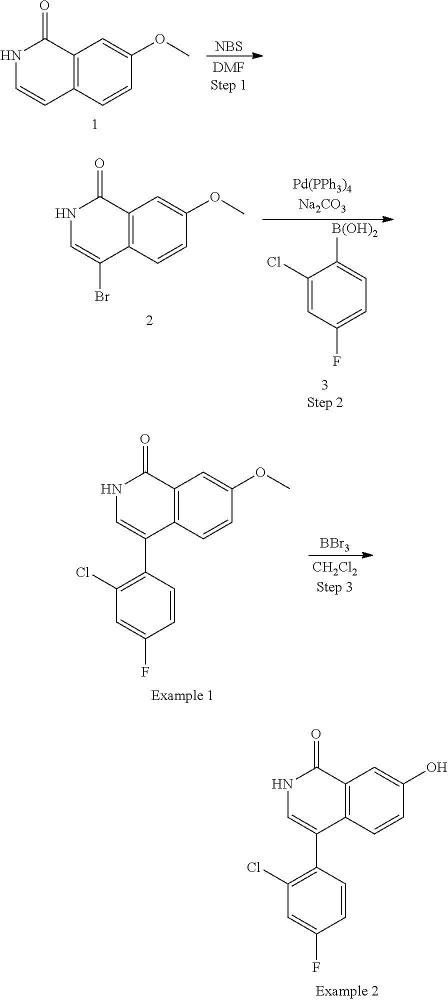

Synthesis of 4-bromo-7-methoxyisoquinolin-1(2H)-one (2) [Step 1]: To a solution of 7-methoxyisoquinolin-1(2H)-one (1, 200 mg, 1.14 mmol) in THF (2 mL) at −5° C. was added a solution of NBS (203 mg, 1.14 mmol) in DMF (1 mL). The reaction mixture was stirred at −5° C. to 0° C. overnight. The reaction mixture was filtered and washed with EtOAc. The combined filtrates were concentrated under reduced pressure. The reaction mixture was diluted with EtOAc and washed with 10% aq. sodium thiosulfate, water, and brine. The reaction mixture was dried over anhydrous Na₂SO₄, filtered, and concentrated under reduced pressure to afford 4-bromo-7-methoxyisoquinolin-1(2H)-one (2, 200 mg). LCMS (ESI) Calcd. for C₁₀H₈BrNO₂: 254, found [M+H]⁺=256.

Synthesis of 4-(2-chloro-4-fluorophenyl)-7-methoxyisoquinolin-1(2H)-one, Example 1 [Step 2]: To a solution of 4-bromo-7-methoxyisoquinolin-1(2H)-one (2, 100 mg, 0.39 mmol) and (2-chloro-4-fluoro-phenyl)boronic acid (3, 103 mg, 0.59 mmol) in 1,4-dioxane (2 mL) at ambient temperature was added a solution of Na₂CO₃ (104 mg, 0.984 mmol) in water (1 mL). The reaction mixture was degassed with nitrogen, and Pd(PPh₃)₄ (45 mg, 0.039 mmol) was added. The reaction mixture was heated at 100° C. for 16 h. The reaction mixture was cooled and concentrated under reduced pressure. The reaction mixture was partitioned between EtOAc and water. The aqueous layer was separated and further extracted with EtOAc (×2). The combined organic layers were washed with brine, dried over anhydrous Na₂SO₄, filtered, and concentrated under reduced pressure. The product was purified by flash column chromatography on silica gel and lyophilized to afford 4-(2-chloro-4-fluorophenyl)-7-methoxyisoquinolin-1(2H)-one (Example 1, 36 mg). LCMS (ESI) Calcd. for C₁₆H₁₁ClFNO₂: 303, found [M+H]⁺=304. ¹H NMR (400 MHz, DMSO-d₆) δ 11.45 (br s, 1H), 7.70 (d, 1H), 7.62 (dd, 1H), 7.50-7.46 (m, 1H), 7.34 (dt, 1H), 7.30 (dd, 1H), 7.00-6.94 (m, 2H), 3.87 (s, 3H).

Synthesis of 4-(2-chloro-4-fluorophenyl)-7-hydroxyisoquinolin-1(2H)-one (Example 2) [Step 3]: To a stirred solution of 4-(2-chloro-4-fluorophenyl)-7-methoxyisoquinolin-1(2H)-one (Example 1, 50 mg, 0.16 mmol) in CH₂Cl₂ (4 mL) at 0° C. was added dropwise BBr₃ (124 mg, 0.49 mmol). The reaction mixture was stirred at 0° C. for 16 h. The reaction mixture was quenched with MeOH and concentrated under reduced pressure. The product was purified by flash column chromatography on silica gel and lyophilized to afford 4-(2-chloro-4-fluorophenyl)-7-hydroxyisoquinolin-1(2H)-one (Example 2, 10 mg). LCMS (ESI) Calcd. for C₁₅H₉ClFNO₂: 289, found [M+H]⁺=290. ¹H NMR (400 MHz, DMSO-d₆) δ 11.29 (s, 1H), 9.99 (s, 1H), 7.64-7.56 (m, 2H), 7.47 (dd, 1H), 7.38-7.28 (m, 1H), 7.13 (dd, 1H), 6.92-6.83 (m, 2H).

### Examples 3-6: Synthesis of 7-methoxy-4-(o-tolyl)isoquinolin-1(2H)-one, 7-isopropoxy-4-(o-tolyl)isoquinolin-1(2H)-one, 7-(sec-butoxy)-4-(o-tolyl)isoquinolin-1(2H)-one, and 7-isobutoxy-4-(o-tolyl)isoquinolin-1(2H)-one

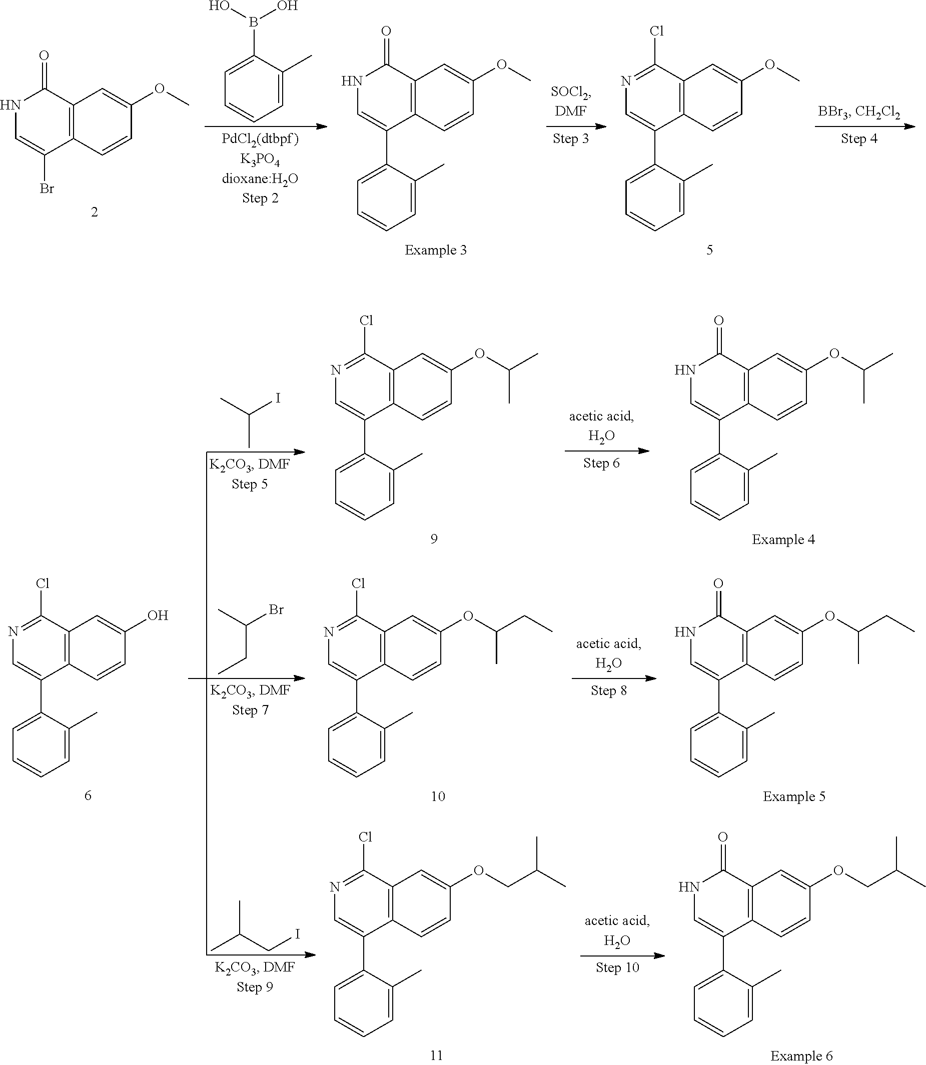

Synthesis of 7-methoxy-4-(o-tolyl)isoquinolin-1(2H)-one, Example 3 [Step 2]: To a solution of 4-bromo-7-methoxyisoquinolin-1(2H)-one (2, 1.0 g, 3.9 mmol) and o-tolyl boronic acid (800 mg, 5.9 mmol) in 1,4-dioxane (12 mL) was added a solution of K₃PO₄ (2.0 g, 9.8 mmol) in water (3 mL). The reaction mixture was degassed with nitrogen prior to the addition of PdCl₂(dtbpf) (260 mg, 0.4 mmol). The reaction mixture was heated to 100° C. for 16 h. The reaction mixture was concentrated under reduced pressure and partitioned between EtOAc and water. The aqueous layer was separated and further extracted with EtOAc (×2). The combined organic layers were washed with brine, dried over anhydrous Na₂SO₄, filtered, and concentrated under reduced pressure. The product was purified by flash column chromatography on silica gel to afford 7-methoxy-4-(o-tolyl)isoquinolin-1(2H)-one (Example 3, 600 mg). LCMS (ESI) Calcd. for C₁₇H₁₅NO₂: 265, found [M+H]⁺=266. ¹H NMR (400 MHz, DMSO-d₆) δ 11.34 (br s, 1H), 7.70 (d, 1H), 7.35-7.34 (m, 2H), 7.30-7.25 (m, 2H), 7.20-7.18 (m, 1H), 6.93-6.87 (m, 2H), 3.86 (s, 3H), 1.98 (s, 3H).

Synthesis of 1-chloro-7-methoxy-4-(o-tolyl)isoquinoline, 5 [Step 3]: To a stirred solution of 7-methoxy-4-(o-tolyl)isoquinolin-1(2H)-one (6, 300 mg, 1.0 mmol) in SOCl₂ (4 mL, 56.5 mmol) under an argon atmosphere was added DMF (0.1 mL, 1 mmol). The reaction mixture was heated to 50° C. for 16 h. The reaction mixture was concentrated under reduced pressure. The reaction mixture was quenched with ice water and extracted with EtOAc (×2). The combined organic extracts were washed with water, brine, dried over anhydrous Na₂SO₄, filtered, and concentrated under reduced pressure. The product was purified by flash column chromatography on silica gel to afford 1-chloro-7-methoxy-4-(o-tolyl)isoquinoline (5, 200 mg). LCMS (ESI) Calcd. for C₁₇H₁₄ClNO: 283, found [M+H]⁺=284. ¹H NMR (400 MHz, DMSO-d₆) δ 8.04 (s, 1H), 7.59 (d, 1H), 7.50 (dd, 1H), 7.43-7.40 (m, 2H), 7.37-7.31 (m, 2H), 7.24 (d, 1H), 3.98 (s, 3H), 1.97 (s, 3H).

Synthesis of 1-chloro-4-(o-tolyl)isoquinolin-7-ol, 8 [Step 4]: To a stirred solution of 1-chloro-7-methoxy-4-(o-tolyl)isoquinoline (5, 50 mg, 0.2 mmol) in CH₂Cl₂ (1 mL) at 0° C. was added dropwise BBr₃ (0.5 mL, 0.5 mmol, 1M in CH₂Cl₂). The reaction mixture was stirred at ambient temperature for 3 h. The reaction mixture was cooled to 0° C. and quenched with MeOH. The reaction mixture was concentrated under reduced pressure to afford 1-chloro-4-(o-tolyl)isoquinolin-7-ol (6, 45 mg). LCMS (ESI) Calcd. for C₁₆H₁₂ClNO: 269, found [M+H]⁺=270.

Synthesis of 1-chloro-7-isopropoxy-4-(o-tolyl)isoquinoline, 9 [Step 5]: To a stirred solution of 1-chloro-4-(o-tolyl)isoquinolin-7-ol (6, 45 mg, 0.1 mmol) in DMF (3 mL) was added K₂CO₃ (58 mg, 0.4 mmol) and 2-iodopropane (0.022 mL, 0.2 mmol). The reaction mixture was heated to 80° C. for 2 h. The reaction mixture was cooled and partitioned between EtOAc and water. The aqueous layer was separated and further extracted with EtOAc. The combined organic layers were washed with water (×3) and brine, dried over anhydrous Na₂SO₄, filtered, and concentrated under reduced pressure. The product was purified by flash column chromatography on silica gel to afford 1-chloro-7-isopropoxy-4-(o-tolyl)isoquinoline (9, 38 mg). LCMS (ESI) Calcd. for C₁₉H₁₈ClNO: 312, found [M+H]⁺=313. ¹H NMR (400 MHz, DMSO-d₆) δ 8.02 (s, 1H), 7.59 (d, 1H), 7.47 (dd, 1H), 7.43-7.42 (m, 2H), 7.35-7.30 (m, 2H), 7.25-7.23 (m, 1H), 4.90-4.87 (m, 1H), 1.97 (s, 3H), 1.37 (d, 6H).

Synthesis of 7-isopropoxy-4-(o-tolyl)isoquinolin-1(2H)-one, Example 4 [Step 6]: To a stirred solution of 1-chloro-7-isopropoxy-4-(o-tolyl)isoquinoline (9, 35 mg, 0.1 mmol) in acetic acid (1 mL, 16.8 mmol) was added water (0.2 mL). The reaction mixture was heated to reflux for 16 h. The reaction mixture was concentrated under reduced pressure. The product was purified by reverse phase prep-HPLC and lyophilized to afford 7-isopropoxy-4-(o-tolyl)isoquinolin-1(2H)-one (Example 4, 14 mg). LCMS (ESI) Calcd. for C₁₉H₁₉NO₂: 293, found [M+H]⁺=294. ¹H NMR (400 MHz, DMSO-d₆) δ 11.33 (br s, 1H), 7.67 (d, 1H), 7.34-7.33 (m, 2H), 7.29-7.18 (m, 3H), 6.90-6.85 (m, 2H), 4.74-4.71 (m, 1H), 2.03 (s, 3H), 1.31 (d, 6H).

Synthesis of 7-(sec-butoxy)-1-chloro-4-(o-tolyl)isoquinoline, 10 [Step 7]: To a stirred solution of 1-chloro-4-(o-tolyl)isoquinolin-7-ol (6, 100 mg, 0.4 mmol) in DMF (1 mL) was added K₂CO₃ (130 mg, 0.9 mmol) and 2-bromobutane (0.05 mL, 0.5 mmol). The reaction mixture was heated to 80° C. for 2 h. The reaction mixture was cooled and partitioned between EtOAc and water. The aqueous layer was separated and extracted further with EtOAc. The combined organic layers were washed with water, brine, dried over anhydrous Na₂SO₄, filtered, and concentrated under reduced pressure. The product was purified by flash column chromatography on silica gel to afford 7-(sec-butoxy)-1-chloro-4-(o-tolyl)isoquinoline (10, 72 mg). LCMS (ESI) Calcd. for C₂₀H₂₀ClNO: 324, found [M+H]⁺=325. ¹H NMR (400 MHz, DMSO-d₆) δ 8.02 (s, 1H), 7.58 (br s, 1H), 7.49 (d, 1H), 7.43-7.41 (m, 2H), 7.34-7.30 (m, 2H), 7.25-7.23 (m, 1H), 4.68-4.63 (m, 1H), 1.97 (s, 3H), 1.77-1.66 (m, 2H), 1.33 (d, 3H), 0.96 (t, 3H).

Synthesis of 7-(sec-butoxy)-4-(o-tolyl)isoquinolin-1(2H)-one, Example 5 [Step 8]: To a stirred solution of 7-(sec-butoxy)-1-chloro-4-(o-tolyl)isoquinoline (10, 72 mg, 0.2 mmol) in acetic acid (1.9 mL, 33 mmol) was added water (0.40 mL). The reaction mixture was heated to reflux for 16 h. The reaction mixture was cooled and concentrated under reduced pressure. The product was purified by reverse phase prep-HPLC and lyophilized to afford 7-(sec-butoxy)-4-(o-tolyl)isoquinolin-1(2H)-one (Example 5, 40 mg). LCMS (ESI) Calcd. for C₂₀H₂₁NO₂: 307, found [M+H]⁺=308. ¹H NMR (400 MHz, DMSO-d₆) δ 11.33 (s, 1H), 7.67 (d, 1H), 7.34-7.33 (m, 2H), 7.29-7.23 (m, 2H), 7.20-7.18 (m, 1H), 6.90-6.86 (m, 2H), 4.52-4.48 (m, 1H), 2.04 (s, 3H), 1.71-1.60 (m, 2H), 1.27 (d, 3H), 0.93 (t, 3H).

Synthesis of 1-chloro-7-isobutoxy-4-(o-tolyl)isoquinoline, 11 [Step 9]: To a stirred solution of 1-chloro-4-(o-tolyl)isoquinolin-7-ol (6, 120 mg, 0.4 mmol) in DMF (2 mL) was added K₂CO₃ (154 mg, 1 mmol) and 1-bromo-2-methyl-propane (0.063 mL, 0.6 mmol). The reaction mixture was heated to 80° C. for 2 h. The reaction mixture was cooled and partitioned between EtOAc and water. The aqueous layer was separated and extracted further with EtOAc. The combined organic layers were washed with water, brine, dried over anhydrous Na₂SO₄, filtered, and concentrated under reduced pressure. The product was purified by flash column chromatography on silica gel to afford 1-chloro-7-isobutoxy-4-(o-tolyl)isoquinoline (11, 81 mg). LCMS (ESI) Calcd. for C₂₀H₂₀ClNO: 324, found [M+H]⁺=326. ¹H NMR (400 MHz, DMSO-d₆) δ 8.03 (s, 1H), 7.57 (br s, 1H), 7.51 (d, 1H), 7.43-7.42 (m, 2H), 7.35-7.30 (m, 2H), 7.25-7.23 (m, 1H), 3.97 (d, 2H), 2.25-2.15 (m, 1H), 1.97 (s, 3H), 1.03 (d, 6H).

Synthesis of 7-isobutoxy-4-(o-tolyl)isoquinolin-1(2H)-one, Example 6 [Step 10]: To a stirred solution of 1-chloro-7-isobutoxy-4-(o-tolyl)isoquinoline (11, 81 mg, 0.25 mmol) in acetic acid (2.1 mL, 37.3 mmol) was added water (0.45 mL). The reaction mixture was heated to reflux for 16 h. The reaction mixture was cooled and concentrated under reduced pressure. The product was purified by reverse phase prep-HPLC and lyophilized to afford 7-isobutoxy-4-(o-tolyl)isoquinolin-1(2H)-one (Example 6, 48 mg). LCMS (ESI) Calcd. for C₂₀H₂₁NO₂: 307, found [M+H]⁺=308. ¹H NMR (400 MHz, DMSO-d₆) δ 11.33 (br s, 1H), 7.68 (br s, 1H), 7.35-7.33 (m, 2H), 7.28-7.26 (m, 2H), 7.20-7.18 (m, 1H), 6.91-6.87 (m, 2H), 3.85 (d, 2H), 2.07-2.03 (m, 4H), 1.00 (d, 6H).

### Example 7: Synthesis of 4-(2-chloro-4-fluorophenyl)-7-isopropoxyisoquinolin-1(2H)-one

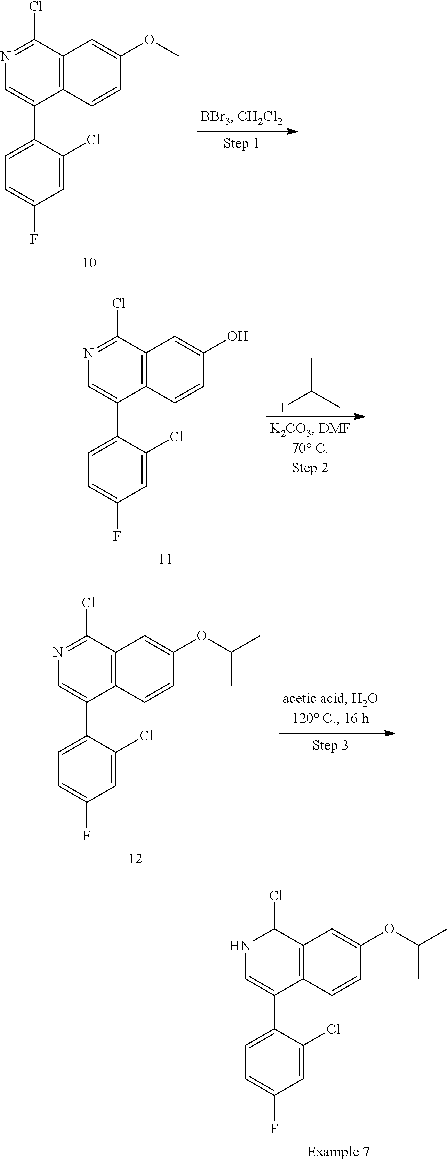

Synthesis of 1-chloro-4-(2-chloro-4-fluorophenyl)isoquinolin-7-ol, 11 [Step 1]: To a stirred solution 1-chloro-4-(2-chloro-4-fluorophenyl)-7-methoxyisoquinoline (10, 280 mg, 0.9 mmol) in CH₂Cl₂ (3 mL) was added dropwise BBr₃ (2.7 mL, 2.7 mmol, 1M in CH₂Cl₂) at 0° C. The reaction mixture was stirred at ambient temperature for 3 h. The reaction mixture was cooled to 0° C. and quenched with MeOH. The reaction mixture was concentrated under reduced pressure to afford 1-chloro-4-(2-chloro-4-fluorophenyl)isoquinolin-7-ol (11, 92 mg). The product was used in the next step without further purification. LCMS (ESI) Calcd. for C₁₅H₈Cl₂FNO: 307, found [M+H]⁺=308. ¹H NMR (400 MHz, DMSO-d₆) δ 10.6 (br s, 1H), 7.99 (br s, 1H), 7.69 (d, 1H), 7.57-7.53 (m, 2H), 7.41-7.30 (m, 3H).

Synthesis of 1-chloro-4-(2-chloro-4-fluorophenyl)-7-isopropoxyisoquinoline, 12 [Step 2]: To a solution of 1-chloro-4-(2-chloro-4-fluorophenyl)isoquinolin-7-ol (11, 85 mg, 0.3 mmol) in DMF (3 mL) was added K₂CO₃ (95 mg, 0.7 mmol) and 2-iodopropane (0.04 mL, 0.4 mmol). The reaction mixture was heated to 70° C. for 2 h. The reaction mixture was partitioned between EtOAc and water. The organic layer was collected, and the aqueous layer was further extracted with EtOAc. The combined organic layers were washed with water, brine, dried over anhydrous Na₂SO₄, filtered, and concentrated under reduced pressure. The product was purified by flash column chromatography to afford 1-chloro-4-(2-chloro-4-fluoro-phenyl)-7-isopropoxyisoquinoline (12, 68 mg). LCMS (ESI) Calcd. for C₁₈H₁₄Cl₂FNO: 349, found [M+H]⁺=349. ¹H NMR (400 MHz, DMSO-d₆) δ 8.07 (s, 1H), 7.70 (dd, 1H), 7.59 (d, 1H), 7.58-7.54 (m, 1H), 7.52-7.49 (m, 1H), 7.45-7.40 (m, 1H), 7.37-7.35 (m, 1H), 4.93-4.87 (m, 1H), 1.37 (d, 6H).

Synthesis of 4-(2-chloro-4-fluorophenyl)-7-isopropoxyisoquinolin-1(2H)-one, Example 7 [Step 3]: To a stirred solution of 1-chloro-4-(2-chloro-4-fluoro-phenyl)-7-isopropoxyisoquinoline (12, 68 mg, 0.2 mmol) in acetic acid (1.7 mL, 29 mmol) was added water (0.35 mL, 19.4 mmol). The reaction mixture was heated to reflux for 16 h. The reaction mixture was concentrated under reduced pressure. The product was purified by prep-HPLC and lyophilized to afford 4-(2-chloro-4-fluorophenyl)-7-isopropoxyisoquinolin-1(2H)-one (Example 7, 50 mg). LCMS (ESI) Calcd. for C₁₈H₁₅ClFNO₂: 331, found [M+H]⁺=332. ¹H NMR (400 MHz, DMSO-d₆) δ 11.42 (br s, 1H), 7.67 (d, 1H), 7.60 (dd, 1H), 7.50-7.46 (m, 1H), 7.36-7.31 (m, 1H), 7.25 (dd, 1H), 6.98-6.96 (m, 1H), 6.94-6.92 (m, 1H), 4.76-4.70 (m, 1H), 1.31 (d, 6H).

### Example 8: Synthesis of N,N-dimethyl-2-((1-oxo-4-(o-tolyl)-1,2-dihydroisoquinolin-7-yl)oxy)acetamide

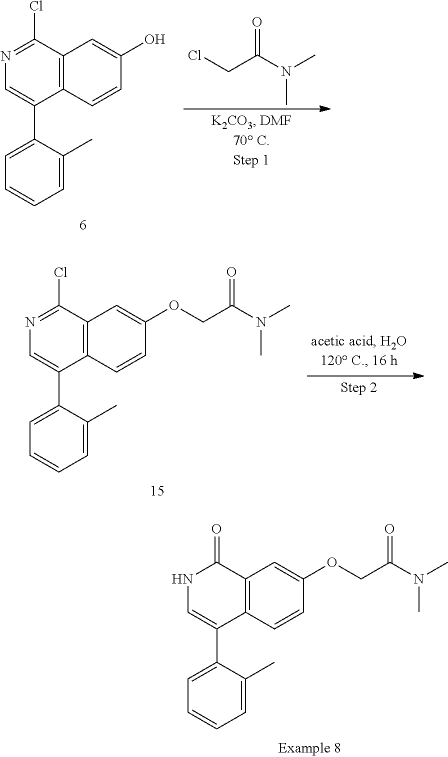

Synthesis of 2-((1-chloro-4-(o-tolyl)isoquinolin-7-yl)oxy)-N,N-dimethylacetamide, 15 [Step 1]: To a stirred solution of 1-chloro-4-(o-tolyl)isoquinolin-7-ol (6, 120 mg, 0.4 mmol) in DMF (1 mL) was added K₂CO₃ (154 mg, 1 mmol) and 2-chloro-N,N-dimethyl-acetamide (70 mg, 0.6 mmol). The reaction mixture was heated at 80° C. for 2 h. The reaction mixture was partitioned between EtOAc and water. The organic layer was collected, and the aqueous layer was further extracted with EtOAc. The combined organic layers were washed with water, brine, dried over anhydrous Na₂SO₄, and concentrated under reduced pressure. The product was purified by flash column chromatography to afford 2-((1-chloro-4-(o-tolyl)isoquinolin-7-yl)oxy)-N,N-dimethylacetamide (15, 105 mg). LCMS (ESI) Calcd. for C₂₀H₁₉ClN₂O₂: 354, found [M+H]⁺=355. ¹H NMR (400 MHz, DMSO-d₆) δ 8.04 (s, 1H), 7.57-7.52 (m, 2H), 7.45-7.41 (m, 2H), 7.37-7.31 (m, 2H), 7.24 (d, 1H), 5.09 (s, 2H), 3.06 (s, 3H), 1.87 (s, 3H), 1.98 (s, 3H).

Synthesis of N,N-dimethyl-2-((1-oxo-4-(o-tolyl)-1,2-dihydroisoquinolin-7-yl)oxy)acetamide, Example 8 [Step 2]: To a stirred solution of 2-((1-chloro-4-(o-tolyl)isoquinolin-7-yl)oxy)-N,N-dimethylacetamide (15, 75 mg, 0.2 mmol) in acetic acid (1.8 mL, 31.7 mmol) was added water (0.4 mL, 21 mmol) at ambient temperature. The reaction mixture was heated to reflux for 16 h. The reaction mixture was concentrated under reduced pressure. The product was purified by reverse phase prep-HPLC and lyophilized to afford N,N-dimethyl-2-((1-oxo-4-(o-tolyl)-1,2-dihydroisoquinolin-7-yl)oxy)acetamide (Example 8, 20 mg). LCMS (ESI) Calcd. for C₂₀H₂₀N₂O₃: 336, found [M+H]⁺=337. ¹H NMR (400 MHz, DMSO-d₆) δ 11.34 (br s, 1H), 7.64 (d, 1H), 7.35 (d, 2H), 7.30-7.26 (m, 2H), 7.19 (d, 1H), 6.91-6.88 (m, 2H), 4.95 (s, 2H), 3.03 (s, 3H), 2.86 (s, 3H), 2.05 (s, 3H).

### Example 9: Synthesis of 7-(2-methoxyethoxy)-4-(o-tolyl)isoquinolin-1(2H)-one

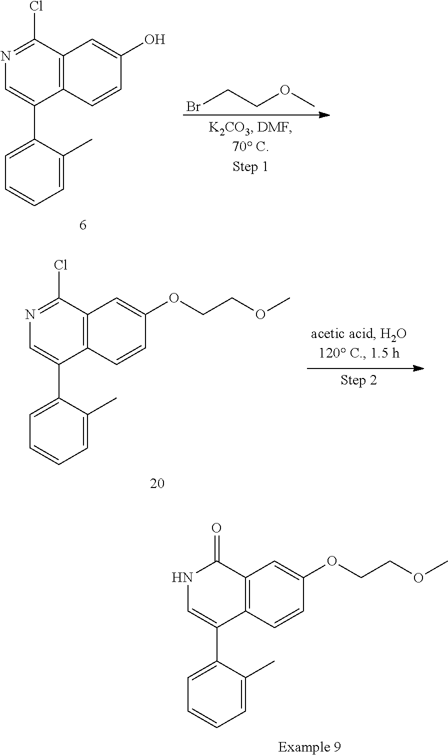

Synthesis of 1-chloro-7-(2-methoxyethoxy)-4-(o-tolyl)isoquinoline, 20 [Step 1]: To a solution of 1-chloro-4-(o-tolyl)isoquinolin-7-ol (6, 120 mg, 0.4 mmol) in DMF (1 mL) was added K₂CO₃ (154 mg, 1 mmol) and 1-bromo-2-methoxy-ethane (80 mg, 0.6 mmol). The reaction mixture was heated to 80° C. for 2 h. The reaction mixture was partitioned between EtOAc and water. The organic layer was collected, and the aqueous layer was further extracted with EtOAc. The combined organic layers were washed with water, brine, dried over anhydrous Na₂SO₄, filtered, and concentrated under reduced pressure. The product was purified by flash column chromatography to afford 1-chloro-7-(2-methoxyethoxy)-4-(o-tolyl)isoquinoline (20, 90 mg). LCMS (ESI) Calcd. for C₁₉H₁₈ClNO₂: 327, found [M+H]⁺=328. ¹H NMR (400 MHz, DMSO-d₆) δ 8.03 (s, 1H), 7.60 (d, 1H), 7.50 (dd, 1H), 7.42-7.40 (m, 2H), 7.35-7.29 (m, 2H), 7.23 (d, 1H), 4.31 (t, 2H), 4.73 (t, 2H), 3.29 (s, 3H), 1.95 (s, 3H).

Synthesis of 7-(2-methoxyethoxy)-4-(o-tolyl)isoquinolin-1(2H)-one, Example 9 [Step 2]: To a stirred solution of 1-chloro-7-(2-methoxyethoxy)-4-(o-tolyl)isoquinoline (20, 90 mg, 0.3 mmol) in acetic acid (2.4 mL, 41.2 mmol) was added water (0.5 mL, 27.5 mmol) at ambient temperature. The reaction mixture was heated to reflux for 16 h. The reaction mixture was concentrated under reduced pressure. The product was purified by reverse phase prep-HPLC and lyophilized to afford 7-(2-methoxyethoxy)-4-(o-tolyl)isoquinolin-1(2H)-one (Example 9, 55 mg). LCMS (ESI) Calcd. for C₁₉H₁₉NO₃: 309, found [M+H]⁺=310. ¹H NMR (400 MHz, DMSO-d₆) δ 11.33 (br s, 1H), 7.70 (d, 1H), 7.35-7.34 (m, 2H), 7.31-7.26 (m, 2H), 7.19 (d, 1H), 6.92-6.87 (m, 2H), 4.20 (t, 2H), 3.70 (t, 2H), 3.28 (s, 3H), 2.04 (s, 3H).

### Example 10: Synthesis of 7-(2-(dimethylamino)ethoxy)-4-(o-tolyl)isoquinolin-1(2H)-one

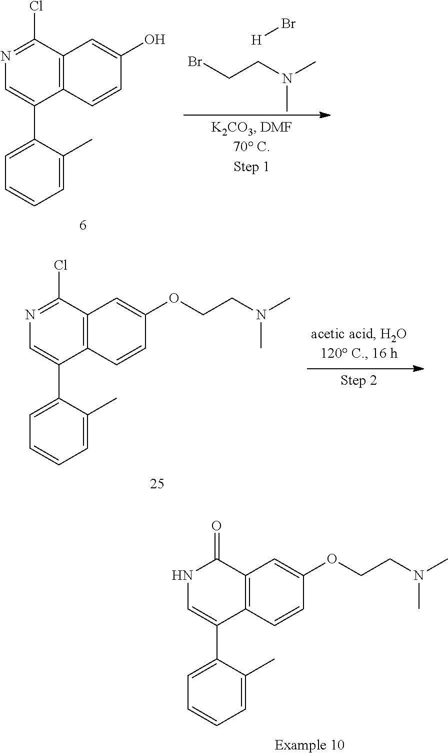

Synthesis of 2-((1-chloro-4-(o-tolyl)isoquinolin-7-yl)oxy)-N,N-dimethylethan-1-amine, 25 [Step 1]: To a stirred solution of 1-chloro-4-(o-tolyl)isoquinolin-7-ol (6, 120 mg, 0.4 mmol) in DMF (1 mL) was added K₂CO₃ (300 mg, 2.2 mmol) and 2-bromo-N,N-dimethyl-ethanamine hydrobromide (135 mg, 0.6 mmol). The reaction mixture was heated at 80° C. for 2 h. The reaction mixture was partitioned between EtOAc and water. The organic layer was collected, and the aqueous layer was further extracted with EtOAc. The combined organic layers were washed with water, brine, dried over anhydrous Na₂SO₄, filtered, and concentrated under reduced pressure. The product was purified by flash column chromatography to afford 2-((1-chloro-4-(o-tolyl)isoquinolin-7-yl)oxy)-N,N-dimethylethan-1-amine (25, 70 mg). LCMS (ESI) Calcd. for C₂₀H₂₁ClN₂O: 340, found [M+H]⁺=341.

Synthesis of 7-(2-(dimethylamino)ethoxy)-4-(o-tolyl)isoquinolin-1(2H)-one, Example 10 [Step 2]: To a stirred solution of 2-((1-chloro-4-(o-tolyl)isoquinolin-7-yl)oxy)-N,N-dimethylethan-1-amine (25, 70 mg, 0.2 mmol) in acetic acid (1.8 mL, 31.7 mmol) was added water (0.38 mL) at ambient temperature. The reaction mixture was heated to reflux for 16 h. The reaction mixture was concentrated under reduced pressure. The product was purified by reverse phase prep-HPLC and lyophilized to afford 7-(2-(dimethylamino)ethoxy)-4-(o-tolyl)isoquinolin-1(2H)-one as a formate salt (Example 10, 15 mg). LCMS (ESI) Calcd. for C₂₀H₂₂N₂O₂: 322, found [M+H]⁺=323. ¹H NMR (400 MHz, DMSO-d₆) δ 11.35 (br s, 1H), 8.32 (br s, 2H), 7.70 (d, 1H), 7.34 (d, 2H), 7.30-7.25 (m, 2H), 7.19 (d, 1H), 6.91-6.88 (m, 2H), 4.15 (t, 2H), 2.66 (t, 2H), 2.23 (s, 6H), 2.03 (s, 3H). The two excess protons are due to the formate salt of the final compound.

### Examples 11-13: Synthesis of 2-((1-oxo-4-(o-tolyl)-1,2-dihydroisoquinolin-7-yl)oxy)propanenitrile and chiral 2-((1-oxo-4-(o-tolyl)-1,2-dihydroisoquinolin-7-yl)oxy)propanenitrile

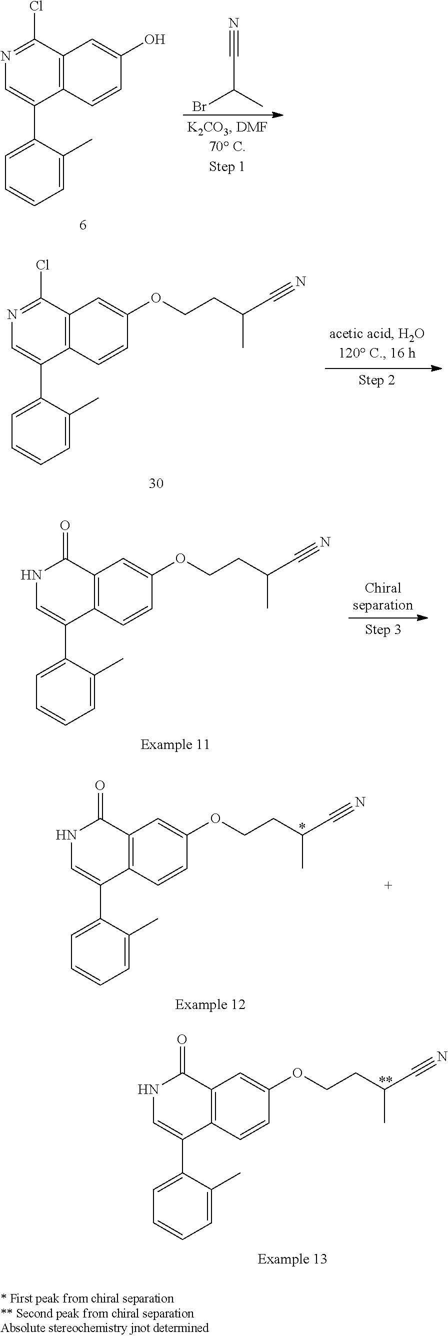

Synthesis of 2-((1-chloro-4-(o-tolyl)isoquinolin-7-yl)oxy)propanenitrile, 30 [Step 1]: To a stirred solution of 1-chloro-4-(o-tolyl)isoquinolin-7-ol (6, 120 mg, 0.4 mmol) in DMF (1 mL) was added K₂CO₃ (154 mg, 1 mmol) followed by 2-bromopropanenitrile (77 mg, 0.6 mmol). The reaction mixture was heated at 80° C. for 2 h. The reaction mixture was partitioned between EtOAc and water, and the organic layer was collected. The aqueous layer was further extracted with EtOAc. The combined organic layers were washed with excess water, brine, dried over anhydrous Na₂SO₄, and concentrated under reduced pressure. The product was purified by flash column chromatography to afford 2-((1-chloro-4-(o-tolyl)isoquinolin-7-yl)oxy)propanenitrile (30, 97 mg). LCMS (ESI) Calcd. for C₁₉H₁₅ClN₂O: 322, found [M+H]⁺=323. ¹H NMR (400 MHz, DMSO-d₆) δ 8.13 (s, 1H), 7.86 (br s, 1H), 7.60 (d, 1H), 7.47-7.36 (m, 4H), 7.28-7.27 (m, 1H), 5.84-5.81 (m, 1H), 1.98 (s, 3H), 1.78 (d, 3H).

Synthesis of 2-((1-oxo-4-(o-tolyl)-1,2-dihydroisoquinolin-7-yl)oxy)propanenitrile, Example 11 [Step 2]: To a stirred solution of 2-((1-chloro-4-(o-tolyl)isoquinolin-7-yl)oxy)propanenitrile (30, 97 mg, 0.3 mmol) in acetic acid (2.6 mL, 45 mmol) was added water (0.54 mL, 30 mmol) at ambient temperature. The reaction mixture was heated to reflux for 16 h. The reaction mixture was concentrated under reduced pressure. The product was purified by prep-HPLC and lyophilized to afford 2-((1-oxo-4-(o-tolyl)-1,2-dihydroisoquinolin-7-yl)oxy)propanenitrile (Example 11, 48 mg).

Synthesis of chiral 2-((1-oxo-4-(o-tolyl)-1,2-dihydroisoquinolin-7-yl)oxy)propanenitrile, Examples 12 and 13 [Step 3]: 2-((1-oxo-4-(o-tolyl)-1,2-dihydroisoquinolin-7-yl)oxy)propanenitrile was purified by chiral prep-HPLC and lyophilized. The first product was isolated as 2-((1-oxo-4-(o-tolyl)-1,2-dihydroisoquinolin-7-yl)oxy)propanenitrile, Peak 1 (Example 12, 12 mg) and the second product as 2-((1-oxo-4-(o-tolyl)-1,2-dihydroisoquinolin-7-yl)oxy)propanenitrile, Peak 2 (Example 13, 15 mg). The absolute stereochemistry for these Examples was not determined.

Example 12: 2-((1-oxo-4-(o-tolyl)-1,2-dihydroisoquinolin-7-yl)oxy)propanenitrile, Peak 1: LCMS (ESI) Calcd. for C₁₉H₁₆N₂O₂: 304, found [M+H]+=305. ¹H NMR (400 MHz, DMSO-d₆) δ 11.46 (br s, 1H), 7.90 (br s, 1H), 7.39-7.35 (m, 3H), 7.31-7.27 (m, 1H), 7.18 (d, 1H), 6.99-6.96 (m, 2H), 5.63 (q, 1H), 2.04 (d, 3H), 1.72 (d, 3H).

Example 13: 2-((1-oxo-4-(o-tolyl)-1,2-dihydroisoquinolin-7-yl)oxy)propanenitrile, Peak 2: LCMS (ESI) Calcd. for C₁₉H₁₆N₂O₂: 304, found [M+H]+=305. ¹H NMR (400 MHz, DMSO-d₆) δ 11.45 (br s, 1H), 7.88 (br s, 1H), 7.36-7.32 (m, 3H), 7.28-7.24 (m, 1H), 7.18 (d, 1H), 6.96-6.94 (m, 2H), 5.62 (q, 1H), 2.02 (d, 3H), 1.70 (d, 3H).

Chiral prep-HPLC: Chiral separation was performed on an Agilent 1200 series instrument. Column was a Chiralpak IC (250×21 mm), 5 μm, operating at ambient temperature with flow rate of 21.0 mL/min. Mobile phase: 80% hexane, 10% CH₂Cl₂, and 10% ethanol, held isocratic for up to 20 min. with detection at 220 nm wavelength.

### Examples 14 and 15: Synthesis of methyl 1-oxo-4-(o-tolyl)-1,2-dihydroisoquinoline-7-carboxylate and 7-(hydroxymethyl)-4-(o-tolyl)isoquinolin-1(2H)-one

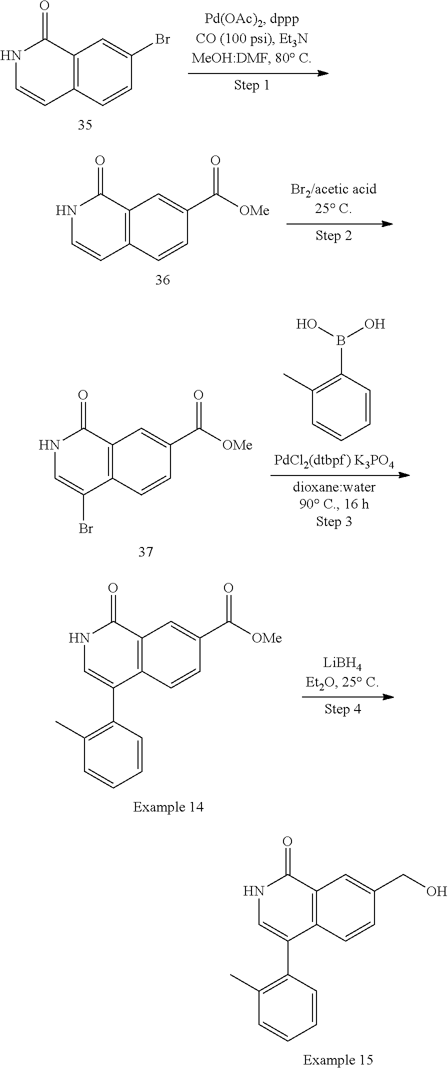

Synthesis of methyl 1-oxo-1,2-dihydroisoquinoline-7-carboxylate, 36 [Step 1]: To a solution of 7-bromoisoquinolin-1(2H)-one (35, 2.0 g, 8.9 mmol), dppp (220 mg, 0.5 mmol), and triethylamine (3.1 mL, 22.3 mmol) in MeOH (20 mL) and DMF (20 mL) was added Pd(OAc)₂ (80 mg, 0.4 mmol) under N₂. The reaction mixture was degassed, purged with CO (×3), and kept at 100 psi CO. The mixture was stirred at 80° C. in an autoclave for 16 h. The reaction mixture was filtered through celite and washed with MeOH. The filtrate was concentrated under reduced pressure to afford methyl 1-oxo-1,2-dihydroisoquinoline-7-carboxylate (36, 1.8 g). The product was used in the next step without further purification. LCMS (ESI) Calcd. for C₁₁H₉NO₃: 203, found [M+H]⁺=204.

Synthesis of methyl 4-bromo-1-oxo-1,2-dihydroisoquinoline-7-carboxylate, 37 [Step 2]: To a mixture of methyl 1-oxo-1,2-dihydroisoquinoline-7-carboxylate (36, 1.8 g, 7.9 mmol) in acetic acid (40 mL) was added Br₂ (0.4 mL, 7.9 mmol). After stirring for 45 min. at 25° C., water was added to the reaction mixture. The reaction mixture was filtered, washed with water, and dried to afford methyl 4-bromo-1-oxo-1,2-dihydroisoquinoline-7-carboxylate (57, 1.60 g). The product was used in the next step without further purification. LCMS (ESI) Calcd. for C₁₁H₈BrNO₃: 281, found [M+H]⁺=282.

Synthesis of methyl 1-oxo-4-(o-tolyl)-1,2-dihydroisoquinoline-7-carboxylate, Example 14 [Step 3]: A mixture of methyl 4-bromo-1-oxo-1,2-dihydroisoquinoline-7-carboxylate (37, 1.0 g, 3.5 mmol), o-tolylboronic acid (720 mg, 5.3 mmol), and K₃PO₄ (1.9 g, 8.9 mmol) in 1,4-dioxane (15 mL) and water (5 mL) was degassed by bubbling Ar for 10 min. PdCl₂(dtbpf) (230 mg, 0.4 mmol) was added, and the reaction mixture was stirred at 90° C. for 16 h. The reaction mixture was cooled to ambient temperature. Water was added, and the reaction mixture was extracted with EtOAc (×2). The combined organic layers were washed with brine, dried over anhydrous Na₂SO₄, filtered, and concentrated under reduced pressure. The product was purified on a silica gel column to afford methyl 1-oxo-4-(o-tolyl)-1,2-dihydroisoquinoline-7-carboxylate (Example 14, 300 mg). LCMS (ESI) Calcd. for C₁₈H₁₅NO₃: 293, found [M+H]⁺=294. ¹H NMR (400 MHz, DMSO-d₆): δ 11.67 (br s, 1H), 8.86 (d, 1H), 8.14 (dd, 1H), 7.38-7.37 (m, 2H), 7.32-7.28 (m, 1H), 7.23 (d, 1H), 7.19 (m, 1H), 7.09 (d, 1H), 3.90 (s, 3H), 2.04 (s, 3H).

Synthesis of 7-(hydroxymethyl)-4-(o-tolyl)isoquinolin-1(2H)-one, Example 15 [Step 4]: To a solution of methyl 1-oxo-4-(o-tolyl)-1,2-dihydroisoquinoline-7-carboxylate (Example 14, 50 mg, 0.2 mmol) in ether (2 mL) was added lithium borohydride solution (0.2 mL, 0.4 mmol, 2M in THF) at 0° C. The reaction mixture was warmed to 25° C. and stirred for 2 h. The reaction mixture was quenched with ice water and extracted with EtOAc (×2). The combined organic layers were washed with brine, dried over anhydrous Na₂SO₄, filtered, and concentrated under reduced pressure. The product was purified by reverse phase prep-HPLC to afford 7-(hydroxymethyl)-4-(o-tolyl)isoquinolin-1(2H)-one (Example 15, 24 mg). LCMS (ESI) Calcd. for C₁₇H₁₅NO₂: 265, found [M+H]⁺=266. ¹H NMR (400 MHz, DMSO-d₆): δ 11.34 (br s, 1H), 8.25 (s, 1H), 7.58 (d, 1H), 7.36-7.35 (m, 2H), 7.31-7.27 (m, 1H), 7.21 (d, 1H), 6.97-6.92 (m, 2H), 5.37 (br s, 1H), 4.61 (s, 2H), 2.04 (s, 3H).

### Example 16: Synthesis of (R)-7-((1-acetylpyrrolidin-3-yl)oxy)-4-(o-tolyl)isoquinolin-1(2H)-one

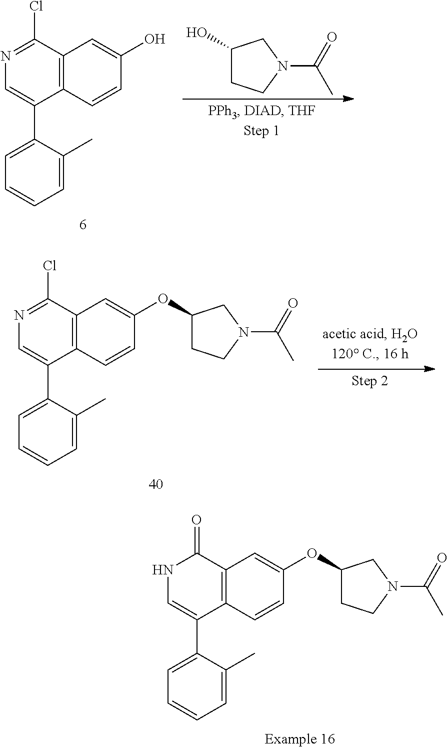

Synthesis of (R)-1-(3-((1-chloro-4-(o-tolyl)isoquinolin-7-yl)oxy)pyrrolidin-1-yl)ethan-1-one, 40 [Step 1]: To a stirred solution of (S)-1-(3-hydroxypyrrolidin-1-yl)ethan-1-one (70 mg, 0.6 mmol) and 1-chloro-4-(o-tolyl)isoquinolin-7-ol (6, 100 mg, 0.4 mmol) in THF (3 mL) was added PPh₃ (292 mg, 1 mmol). DIAD (0.2 mL, 1 mmol) was added to the reaction mixture at 0° C. The reaction mixture was stirred at 80° C. for 16 h. The reaction mixture was partitioned between EtOAc and water. The organic layer was collected, and the aqueous layer was further extracted with EtOAc (×3). The combined organic layers were washed with water, brine, dried over anhydrous Na₂SO₄, filtered, and concentrated under reduced pressure. The product was purified by flash column chromatography to afford (R)-1-(3-((1-chloro-4-(o-tolyl)isoquinolin-7-yl)oxy)pyrrolidin-1-yl)ethan-1-one (40, 120 mg). LCMS (ESI) Calcd. for C₂₂H₂₁ClN₂O₂: 380, found [M+H]⁺=381.

Synthesis of (R)-7-((1-acetylpyrrolidin-3-yl)oxy)-4-(o-tolyl)isoquinolin-1(2H)-one, Example 16 [Step 2]: To a stirred solution of (R)-1-(3-((1-chloro-4-(o-tolyl)isoquinolin-7-yl)oxy)pyrrolidin-1-yl)ethan-1-one (40, 120 mg, 0.3 mmol) in acetic acid (2.7 mL, 47.3 mmol) was added water (0.6 mL). The reaction mixture was heated to reflux for 16 h. The reaction mixture was concentrated under reduced pressure. The product was purified by reverse phase prep-HPLC and lyophilized to afford (R)-7-((1-acetylpyrrolidin-3-yl)oxy)-4-(o-tolyl)isoquinolin-1(2H)-one (Example 16, 40 mg). LCMS (ESI) Calcd. for C₂₂H₂₂N₂O₃: 362, found [M+H]⁺=363. ¹H NMR (400 MHz, DMSO-d₆) δ 11.39 (br s, 1H), 7.70 (dd, 1H), 7.35 (d, 2H), 7.30-7.26 (m, 2H), 7.19 (d, 1H), 6.93-6.90 (m, 2H), 5.24-5.16 (m, 1H) 3.63-3.54 (m, 4H), 2.28-2.10 (m, 2H), 2.04 (s, 3H), 1.97-1.93 (m, 3H).

### Example 17: Synthesis of (S)-7-((1-acetylpyrrolidin-3-yl)oxy)-4-(o-tolyl)isoquinolin-1(2H)-one

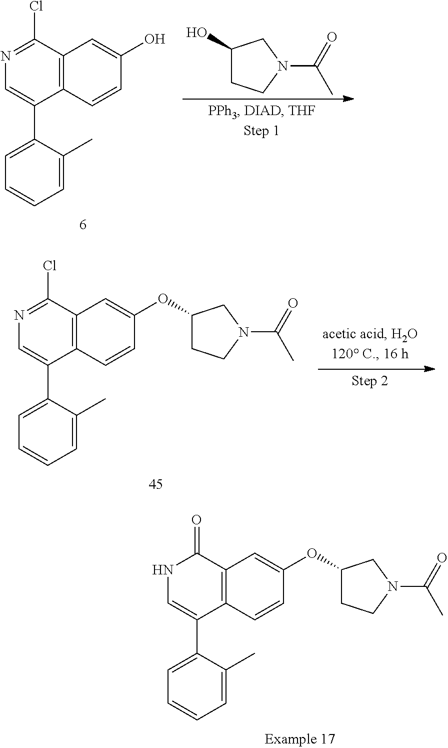

Synthesis of (S)-1-(3-((1-chloro-4-(o-tolyl)isoquinolin-7-yl)oxy)pyrrolidin-1-yl)ethan-1-one, 45 [Step 1]: To a stirred solution of (R)-1-(3-hydroxypyrrolidin-1-yl)ethan-1-one (70 mg, 0.6 mmol) and 1-chloro-4-(o-tolyl)isoquinolin-7-ol (6, 100 mg, 0.4 mmol) in THF (3 mL) was added PPh₃ (290 mg, 1 mmol). DIAD (0.2 mL, 1.1 mmol) was added to the reaction mixture at 0° C. The reaction mixture was heated at 80° C. for 16 h. The reaction mixture was partitioned between EtOAc and water. The organic layer was collected, and the aqueous layer was further extracted with EtOAc (×3). The combined organic layers were washed with water, brine, dried over anhydrous Na₂SO₄, filtered, and concentrated under reduced pressure. The product was purified by flash column chromatography to afford (S)-1-(3-((1-chloro-4-(o-tolyl)isoquinolin-7-yl)oxy)pyrrolidin-1-yl)ethan-1-one (45, 135 mg). LCMS (ESI) Calcd. for C₂₂H₂₁ClN₂O₂: 380, found [M+H]⁺=381.

Synthesis of (S)-7-((1-acetylpyrrolidin-3-yl)oxy)-4-(o-tolyl)isoquinolin-1(2H)-one, Example 17 [Step 2]: To a stirred solution of (S)-1-(3-((1-chloro-4-(o-tolyl)isoquinolin-7-yl)oxy)pyrrolidin-1-yl)ethan-1-one (45, 135 mg, 0.4 mmol) in acetic acid (3.0 mL, 53.2 mmol) was added water (0.64 mL). The reaction mixture was heated to reflux for 16 h. The reaction mixture was concentrated under reduced pressure. The product was purified by reverse phase prep-HPLC and lyophilized to afford (S)-7-((1-acetylpyrrolidin-3-yl)oxy)-4-(o-tolyl)isoquinolin-1(2H)-one (Example 17, 40 mg). LCMS (ESI) Calcd. for C₂₂H₂₂N₂O₃: 362, found [M+H]⁺=363. ¹H NMR (400 MHz, DMSO-d₆) δ 11.39 (br s, 1H), 7.70 (dd, 1H), 7.35 (d, 2H), 7.30-7.26 (m, 2H), 7.19 (d, 1H), 6.93-6.90 (m, 2H), 5.24-5.16 (m, 1H), 3.63-3.54 (m, 4H), 2.28-2.10 (m, 2H), 2.04 (s, 3H), 1.97-1.93 (m, 3H).

### Examples 18-19: Synthesis of 1-oxo-4-(o-tolyl)-1,2-dihydroisoquinoline-7-carboxylic acid and 7-(pyrrolidine-1-carbonyl)-4-(o-tolyl)isoquinolin-1(2H)-one

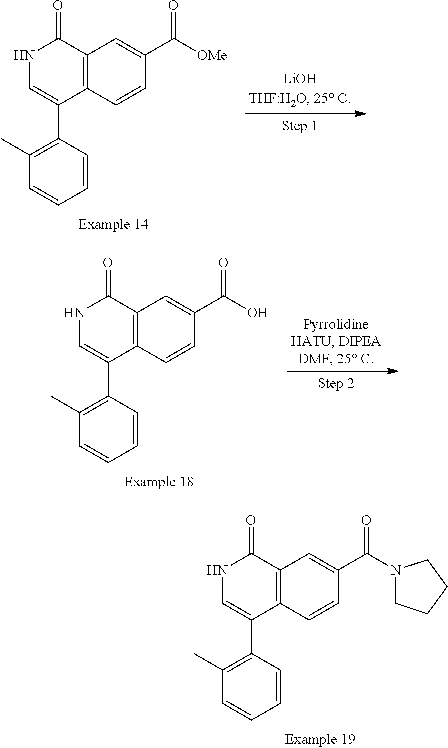 [SMILES: COC(=O)c1ccc2c(-c3ccccc3C)c[nH]c(=O)c2c1 ; Cc1ccccc1-c1c[nH]c(=O)c2cc(C(=O)O)ccc12 ; Cc1ccccc1-c1c[nH]c(=O)c2cc(C(=O)N3CCCC3)ccc12]

Synthesis of 1-oxo-4-(o-tolyl)-1,2-dihydroisoquinoline-7-carboxylic acid, Example 18 [Step 1]: To a solution of methyl 1-oxo-4-(o-tolyl)-1,2-dihydroisoquinoline-7-carboxylate (Example 14, 200 mg, 0.7 mmol) in THF (5 mL) and water (1 mL) was added lithium hydroxide (24 mg, 1.0 mmol). The reaction mixture was stirred at 25° C. for 2 h. The reaction mixture was partitioned between EtOAc and water. The aqueous phase was acidified with 1N HCl to pH ˜2. The product was filtered, washed with water, and dried to afford 1-oxo-4-(o-tolyl)-1,2-dihydroisoquinoline-7-carboxylic acid (Example 18, 50 mg). The product was used in the next step without further purification. LCMS (ESI) Calcd. for C₁₇H₁₃NO₃: 279, found [M+H]⁺=280.

Synthesis of 7-(pyrrolidine-1-carbonyl)-4-(o-tolyl)isoquinolin-1(2H)-one, Example 19 [Step 2]: To a solution of 1-oxo-4-(o-tolyl)-1,2-dihydroisoquinoline-7-carboxylic acid (Example 18, 50 mg, 0.18 mmol) in DMF (1.5 mL) was added DIPEA (75 μL, 0.5 mmol) and HATU (102 mg, 0.27 mmol). The reaction mixture was stirred for 30 min., and a solution of pyrrolidine (20 μL, 0.2 mmol) in DMF (0.5 mL) was added. The reaction mixture was stirred for 16 h. at 25° C. The reaction mixture was partitioned between EtOAc and water. The organic phase was separated, washed with brine, dried over anhydrous Na₂SO₄, filtered, and concentrated under reduced pressure. The product was purified by reverse phase prep-HPLC and lyophilized to afford 7-(pyrrolidine-1-carbonyl)-4-(o-tolyl)isoquinolin-1(2H)-one (Example 19, 15 mg). LCMS (ESI) Calcd. for C₂₁H₂₀N₂O₂: 332, found [M+H]⁺=333. ¹H NMR (400 MHz, DMSO-d₆): δ 11.55 (br s, 1H), 8.39-8.38 (m, 1H), 7.78 (dd, 1H), 7.37-7.36 (m, 2H), 7.31-7.28 (m, 1H), 7.24-7.22 (m, 1H), 7.10 (s, 1H), 7.01 (m, 1H), 3.49 (t, 2H), 3.42 (t, 2H), 2.36 (s, 3H), 1.90-1.80 (m, 4H).

### Example 20: Synthesis of (R)—N-methyl-2-((1-oxo-4-(o-tolyl)-1,2-dihydroisoquinolin-7-yl)oxy)propenamide

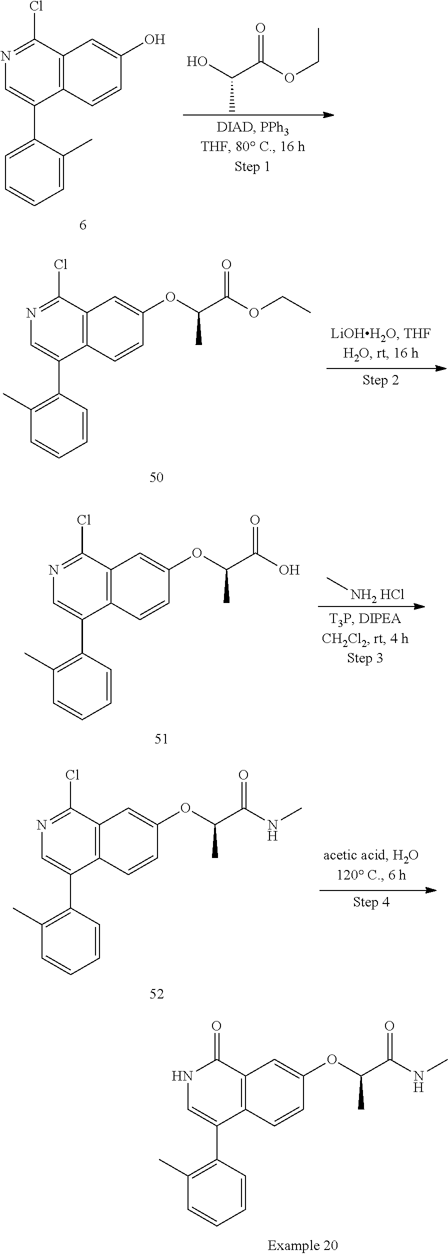

Synthesis of ethyl (R)-2-((1-chloro-4-(o-tolyl)isoquinolin-7-yl)oxy)propanoate, 50 [Step 1]: To a stirred solution of 1-chloro-4-(o-tolyl)isoquinolin-7-ol (6, 220 mg, 0.8 mmol) and ethyl (S)-2-hydroxypropanoate (0.14 mL, 1.2 mmol) in THF (5 mL) was added PPh₃ (640 mg, 2.4 mmol) and DIAD (0.5 mL, 2.4 mmol) at 0° C. under an inert atmosphere. 4 Å molecular sieves (200 mg) were added to the reaction mixture. The reaction mixture was heated at 80° C. for 16 h. The reaction mixture was concentrated under reduced pressure. The product was partitioned between EtOAc and water. The organic phase was separated, washed with brine, dried over anhydrous Na₂SO₄, filtered, and concentrated under reduced pressure. The product was purified by flash column chromatography to afford ethyl (R)-2-((1-chloro-4-(o-tolyl)isoquinolin-7-yl)oxy)propanoate (50, 300 mg). LCMS (ESI) Calcd. for C₂₁H₂₀ClNO₃: 369, found [M+H]⁺=370. ¹H NMR (400 MHz, DMSO-d₆) δ 8.06 (d, 1H), 7.55 (d, 1H), 7.50 (d, 1H), 7.45-7.42 (m, 2H), 7.37-7.33 (m, 2H), 7.26-7.24 (t, 1H), 5.27-5.15 (m, 1H), 4.25-4.15 (m, 2H), 1.97-1.89 (dd, 3H), 1.60 (d, 3H), 1.25-1.17 (m, 3H).

Synthesis of (R)-2-((1-chloro-4-(o-tolyl)isoquinolin-7-yl)oxy)propanoic acid, 51 [Step 2]: To a stirred solution of ethyl (R)-2-((1-chloro-4-(o-tolyl)isoquinolin-7-yl)oxy)propanoate (50, 300 mg, 0.8 mmol) in THF (3 mL) and water (1 mL) was added LiOH·H₂O (140 mg, 3.2 mmol). The reaction mixture was stirred for 2 h. at ambient temperature. The product was diluted with water, acidified with 10% aq. citric acid solution, and extracted with EtOAc (×3). The combined organic layers were washed with brine, dried over anhydrous Na₂SO₄, filtered, and concentrated under reduced pressure to afford (R)-2-((1-chloro-4-(o-tolyl)isoquinolin-7-yl)oxy)propanoic acid (51, 220 mg). LCMS (ESI) Calcd. for C₁₉H₁₆ClNO₃: 341, found [M+H]⁺=342.

Synthesis of (R)-2-((1-chloro-4-(o-tolyl)isoquinolin-7-yl)oxy)-N-methylpropanamide, 52 [Step 3]: To a stirred solution of (R)-2-((1-chloro-4-(o-tolyl)isoquinolin-7-yl)oxy)propanoic acid (51, 50 mg, 0.2 mmol) in CH₂Cl₂ (5 mL) was added DIPEA (0.1 mL, 0.4 mmol) and methylamine hydrochloride (50 mg, 0.2 mmol). T₃P (0.13 mL, 0.2 mmol, 50% in EtOAc) was added to the reaction mixture at 0° C. The reaction mixture was stirred at ambient temperature for 4 h. The reaction mixture was concentrated under reduced pressure and partitioned between EtOAc and water. The organic phase was separated, washed with brine, dried over anhydrous Na₂SO₄, filtered, and concentrated under reduced pressure. The product was purified by flash chromatography to afford (R)-2-((1-chloro-4-(o-tolyl)isoquinolin-7-yl)oxy)-N-methylpropanamide (52, 48 mg). LCMS (ESI) Calcd. for C₂₀H₁₉ClN₂O₂: 354, found [M+H]⁺=355.

Synthesis of (R)—N-methyl-2-((1-oxo-4-(o-tolyl)-1,2-dihydroisoquinolin-7-yl)oxy)propenamide, Example 20 [Step 4]: To a stirred solution of (R)-2-((1-chloro-4-(o-tolyl)isoquinolin-7-yl)oxy)-N-methylpropanamide (52, 50 mg, 0.14 mmol) in acetic acid (1.2 mL, 21.1 mmol) was added water (0.3 mL) at ambient temperature. The reaction mixture was heated to reflux for 16 h. The reaction mixture was concentrated under reduced pressure and partitioned between EtOAc and water. The organic phase was separated, washed with brine, dried over anhydrous Na₂SO₄, filtered, and concentrated under reduced pressure. The product was purified via reverse phase prep-HPLC and lyophilized to afford (R)—N-methyl-2-((1-oxo-4-(o-tolyl)-1,2-dihydroisoquinolin-7-yl)oxy)propanamide (Example 20, 24 mg). LCMS (ESI) Calcd. for C₂₀H₂₀N₂O₃: 336, found [M+H]⁺=337. ¹H NMR (400 MHz, DMSO-d₆) δ 11.36 (d, 1H), 8.15-8.11 (m, 1H), 7.65 (d, 1H), 7.35 (d, 2H), 7.30-7.27 (m, 2H), 7.19 (d, 1H), 6.92-6.88 (m, 2H), 4.80 (d, 1H), 2.61-2.59 (m, 3H), 2.04 (d, 3H), 1.46 (d, 3H).

### Examples 21-22: Synthesis of ethyl (S)-1-((R)-2-((1-oxo-4-(o-tolyl)-1,2-dihydroisoquinolin-7-yl)oxy)propanoyl)piperidine-3-carboxylate and (S)-1-((R)-2-((1-oxo-4-(o-tolyl)-1,2-dihydroisoquinolin-7-yl)oxy)propanoyl)piperidine-3-carboxylic acid

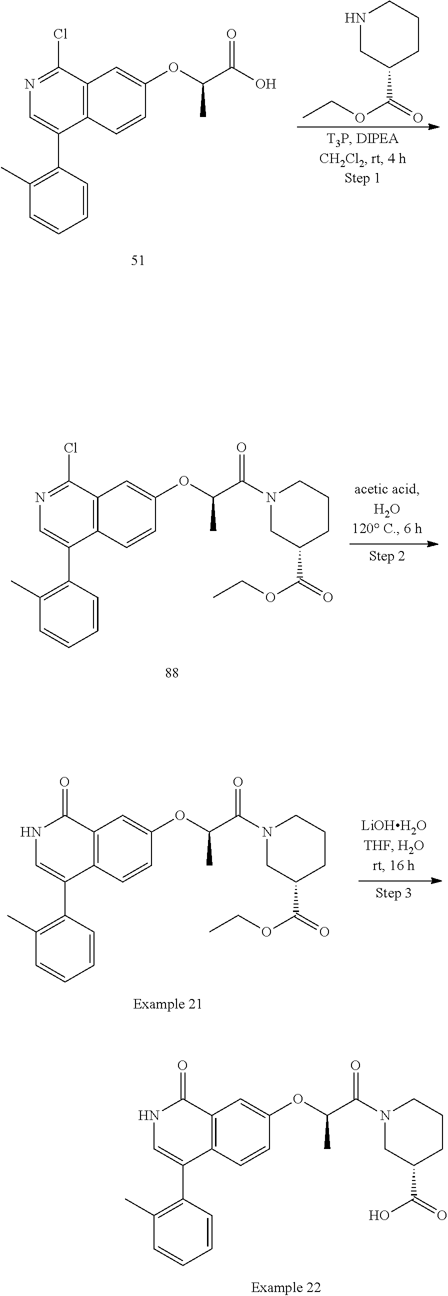 [SMILES: Cc1ccccc1-c1cnc(Cl)c2cc(O[C@H](C)C(=O)O)ccc12 ; CCOC(=O)[C@H]1CCCNC1 ; CCOC(=O)[C@H]1CCCN(C(=O)[C@@H](C)Oc2ccc3c(-c4ccccc4C)cnc(Cl)c3c2)C1 ; CCOC(=O)[C@H]1CCCN(C(=O)[C@@H](C)Oc2ccc3c(-c4ccccc4C)c[nH]c(=O)c3c2)C1 ; Cc1ccccc1-c1c[nH]c(=O)c2cc(O[C@H](C)C(=O)N3CCC[C@H](C(=O)O)C3)ccc12]

Synthesis of ethyl (S)-1-((R)-2-((1-chloro-4-(o-tolyl)isoquinolin-7-yl)oxy)propanoyl)piperidine-3-carboxylate, 55 [Step 1]: To a stirred solution of (R)-2-((1-chloro-4-(o-tolyl)isoquinolin-7-yl)oxy)propanoic acid (51, 125 mg, 0.4 mmol) in CH₂Cl₂ (5 mL) was added DIPEA (0.15 mL, 1.1 mmol) followed by ethyl (S)-piperidine-3-carboxylate (0.2 mL, 1.1 mmol). T₃P (0.3 mL, 0.6 mmol, 50% in EtOAc) was added at 0° C. The reaction mixture was stirred at ambient temperature for 4 h. The reaction mixture was concentrated under reduced pressure and partitioned between EtOAc and water. The organic phase was separated, washed with brine, dried over anhydrous Na₂SO₄, filtered, and concentrated under reduced pressure. The product was purified by flash chromatography to afford ethyl (S)-1-((R))-2-((1-chloro-4-(o-tolyl)isoquinolin-7-yl)oxy)propanoyl)piperidine-3-carboxylate (55, 150 mg). LCMS (ESI) Calcd. for C₂₇H₂₉ClN₂O₄: 480, found [M+H]⁺=481.

Synthesis of ethyl (S)-1-((R)-2-((1-oxo-4-(o-tolyl)-1,2-dihydroisoquinolin-7-yl)oxy)propanoyl)piperidine-3-carboxylate, Example 21 [Step 2]: To a stirred solution of ethyl (S)-1-((R))-2-((1-chloro-4-(o-tolyl)isoquinolin-7-yl)oxy)propanoyl)piperidine-3-carboxylate (55, 210 mg, 0.4 mmol) in acetic acid (4 mL, 66 mmol) was added water (0.8 mL) at ambient temperature. The reaction mixture was heated to reflux for 16 h. The reaction mixture was concentrated under reduced pressure and partitioned between EtOAc and water. The organic phase was separated, washed with brine, dried over anhydrous Na₂SO₄, filtered, and concentrated under reduced pressure to afford ethyl (S)-1-((R)-2-((1-oxo-4-(o-tolyl)-1,2-dihydroisoquinolin-7-yl)oxy)propanoyl)piperidine-3-carboxylate (Example 21, 140 mg). LCMS (ESI) Calcd. for C₂₇H₃₀N₂O₅: 462, found [M+H]⁺=464.

Synthesis of (S)-1-((R)-2-((1-oxo-4-(o-tolyl)-1,2-dihydroisoquinolin-7-yl)oxy)propanoyl)piperidine-3-carboxylic acid, Example 22 [Step 3]: To a stirred solution of ethyl (S)-1-((R)-2-((1-oxo-4-(o-tolyl)-1,2-dihydroisoquinolin-7-yl)oxy)propanoyl)piperidine-3-carboxylate (Example 21, 140 mg, 0.3 mmol) in THF (1 mL) and water (0.3 mL) was added LiOH·H₂O (50 mg, 1.2 mmol) at ambient temperature. The reaction mixture stirred for 2 h. The reaction mixture was diluted with water, acidified with 10% aq. citric acid solution, and extracted with EtOAc (×3). The combined organic layers were washed with brine, dried over anhydrous Na₂SO₄, filtered, and concentrated under reduced pressure. The product was purified via reverse phase prep-HPLC and lyophilized to afford (S)-1-((R)-2-((1-oxo-4-(o-tolyl-1,2-dihydroisoquinolin-7-yl)oxy)propanoyl)piperidine-3-carboxylic acid (Example 22, 25 mg). LCMS (ESI) Calcd. for C₂₅H₂₆N₂O₅: 434, found [M+H]⁺=435. ¹H NMR (400 MHz, DMSO-d₆) 100° C. δ 7.67 (br s, 1H), 7.34-7.18 (m, 5H), 6.90 (d, 1H), 6.83 (s, 1H), 5.36 (d, 1H), 4.30-3.91 (m, 4H), 2.32 (br s, 1H), 2.06 (br s, 4H), 1.91 (s, 2H), 1.69 (br s, 2H), 1.48 (d, 3H).

### Example 23: Synthesis of N,N-dimethyl-1-oxo-4-(o-tolyl)-1,2-dihydroisoquinoline-7-carboxamide

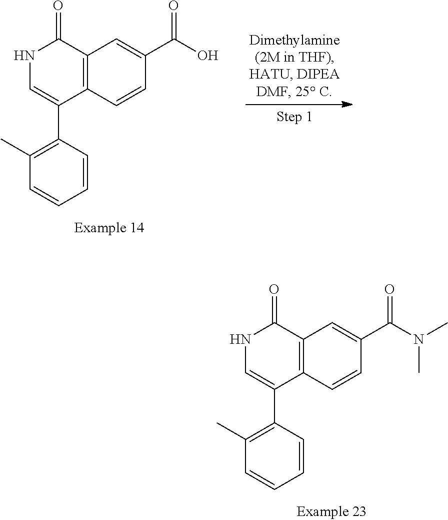

Synthesis of N,N-dimethyl-1-oxo-4-(o-tolyl)-1,2-dihydroisoquinoline-7-carboxamide, Example 23 [Step 1]: An oven-dried round bottom flask was charged with 1-oxo-4-(o-tolyl)-1,2-dihydroisoquinoline-7-carboxylic acid (Example 14, 150 mg, 0.5 mmol). DMF (2 mL), HATU (224 mg, 0.6 mmol), DIPEA (0.1 mL, 0.8 mmol), and dimethylamine (2M in THF) (0.3 mL, 0.6 mmol) were added to the reaction mixture. The reaction mixture was stirred at 25° C. for 16 h. The reaction mixture was partitioned between EtOAc and water. The organic phase was collected, washed with cold water (×3) and brine, dried over anhydrous Na₂SO₄, filtered, and concentrated under reduced pressure. The product was purified by reverse phase prep-HPLC to afford N,N-dimethyl-1-oxo-4-(o-tolyl)-1,2-dihydroisoquinoline-7-carboxamide (Example 23, 28 mg). LCMS (ESI) Calcd. for C₁₉H₁₈N₂O₂: 306, found [M−H]⁻=305. ¹H NMR (400 MHz, DMSO-d₆): δ 11.55 (d, 1H), 8.26-8.25 (m, 1H), 7.69-7.66 (m, 1H), 7.38-7.36 (m, 2H), 7.32-7.29 (m, 1H), 7.24-7.21 (m, 1H), 7.11-7.09 (m, 1H), 7.01 (d, 1H), 2.97 (m, 6H), 2.06 (s, 3H).

### Example 24: Synthesis of (R)-1-(2-((1-oxo-4-(o-tolyl)-1,2-dihydroisoquinolin-7-yl)oxy)propanoyl)piperidine-4-carboxylic acid

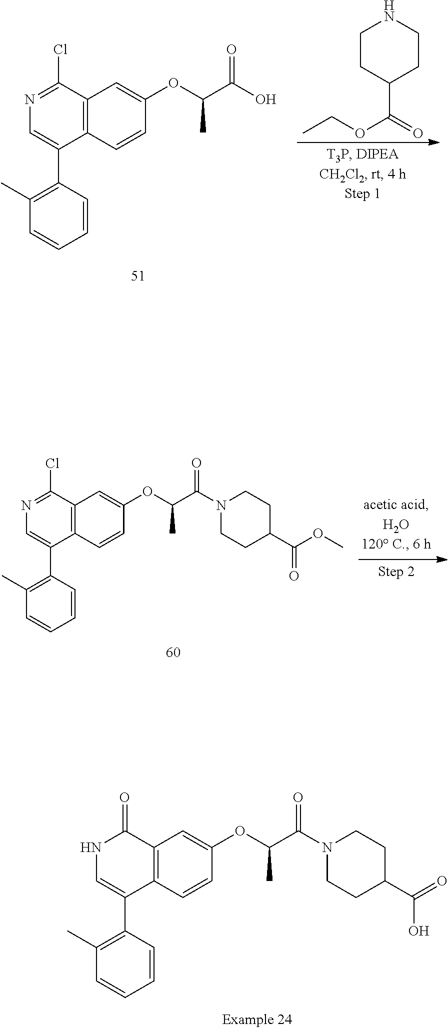

Synthesis of methyl (R)-1-(2-((1-chloro-4-(o-tolyl)isoquinolin-7-yl)oxy)propanoyl)piperidine-4-carboxylate, 60 [Step 1]: To a stirred solution of (R)-2-((1-chloro-4-(o-tolyl)isoquinolin-7-yl)oxy)propanoic acid (51, 100 mg, 0.3 mmol) in CH₂Cl₂ (5 mL) was added DIPEA (0.1 mL, 0.9 mmol) followed by methyl piperidine-4-carboxylate (84 mg, 0.6 mmol). T₃P (0.5 mL, 0.9 mmol, 50% in EtOAc) was added at 0° C. The reaction mixture was stirred at ambient temperature for 16 h. The reaction mixture was quenched with water and extracted with CH₂Cl₂. The combined organic layers were washed with water, brine, dried over anhydrous Na₂SO₄, filtered, and concentrated under reduced pressure to afford methyl (R)-1-(2-((1-chloro-4-(o-tolyl)isoquinolin-7-yl)oxy)propanoyl)piperidine-4-carboxylate (60, 135 mg). LCMS (ESI) Calcd. for C₂₆H₂₇ClN₂O₄: 466, found [M+H]⁺=467.

Synthesis of (R)-1-(2-((1-oxo-4-(o-tolyl)-1,2-dihydroisoquinolin-7-yl)oxy)propanoyl)piperidine-4-carboxylic acid, Example 24 [Step 2]: To a stirred solution of methyl (R)-1-(2-((1-chloro-4-(o-tolyl)isoquinolin-7-yl)oxy)propanoyl)piperidine-4-carboxylate (60, 160 mg, 0.3 mmol) in acetic acid (3.0 mL, 51.4 mmol) was added water (0.6 mL) at ambient temperature. The reaction mixture was heated to reflux for 16 h. The reaction mixture was concentrated under reduced pressure. The product was purified via reverse phase prep-HPLC and lyophilized to afford (R)-1-(2-((1-oxo-4-(o-tolyl)-1,2-dihydroisoquinolin-7-yl)oxy)propanoyl)piperidine-4-carboxylic acid (Example 24, 40 mg). LCMS (ESI) Calcd. for C₂₅H₂₆N₂O₅: 434, found [M+H]⁺=435. ¹H NMR (400 MHz, DMSO-d₆) δ 12.37 (br s, 1H), 11.31 (s, 1H), 7.59 (s, 1H), 7.33 (d, 2H), 7.31-7.17 (m, 3H), 6.90 (t, 2H), 5.41 (br s, 1H), 4.22-4.01 (m, 3H), 3.25 (br s, 1H), 2.92 (m, 1H), 2.78-2.67 (m, 1H), 2.03 (s, 3H), 1.91-1.83 (m, 2H), 1.72 (m, 1H), 1.44 (t, 3H).

### Examples 25-27: Synthesis of 7-((1-methoxypropan-2-yl)oxy)-4-(o-tolyl)isoquinolin-1(2H)-one and chiral 7-((1-methoxypropan-2-yl)oxy)-4-(o-tolyl)isoquinolin-1(2H)-one

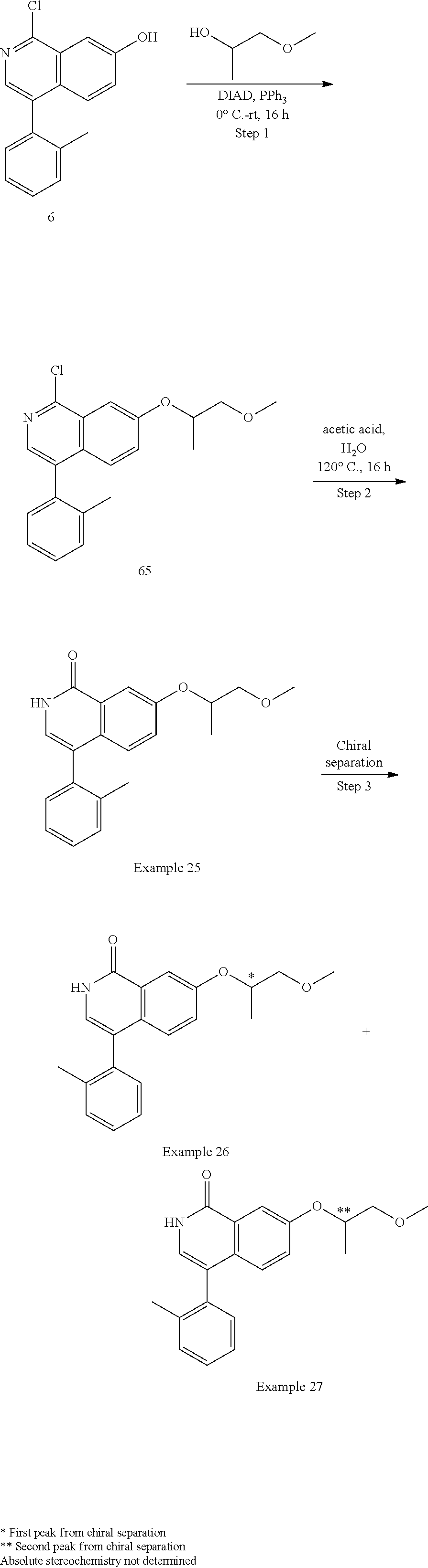

Synthesis of 1-chloro-7-((1-methoxypropan-2-yl)oxy)-4-(o-tolyl)isoquinoline, 65 [Step 1]: To a stirred solution of 1-chloro-4-(o-tolyl)isoquinolin-7-ol (6, 100 mg, 0.4 mmol) in THF (5 mL) was added 1-methoxypropan-2-ol (50 mg, 0.6 mmol) followed by PPh₃ (290 mg, 1.1 mmol) and 4 Å MS (200 mg). DIAD (0.2 mL, 1.1 mmol) was added at 0° C. The reaction mixture was stirred at ambient temperature for 16 h. The reaction mixture was filtered, diluted with EtOAc, washed with water, brine, dried over anhydrous Na₂SO₄, filtered, and concentrated under reduced pressure. The product was purified by flash chromatography to afford 1-chloro-7-((1-methoxypropan-2-yl)oxy)-4-(o-tolyl)isoquinoline (65, 90 mg). LCMS (ESI) Calcd. for C₂₀H₂₀ClNO₂: 341, found [M+H]⁺=342.

Synthesis of 7-((1-methoxypropan-2-yl)oxy)-4-(o-tolyl)isoquinolin-1(2H)-one, Example 25 [Step 2]: To a stirred solution of 1-chloro-7-((1-methoxypropan-2-yl)oxy)-4-(o-tolyl)isoquinoline (65, 120 mg, 0.4 mmol) in acetic acid (3.0 mL, 53 mmol) was added water (0.6 mL). The reaction mixture was heated to reflux for 16 h. The reaction mixture was concentrated under reduced pressure. The product was purified via reverse phase prep-HPLC and lyophilized to afford 7-((1-methoxypropan-2-yl)oxy)-4-(o-tolyl)isoquinolin-1(2H)-one (Example 25, 60 mg). LCMS (ESI) Calcd. for C₂₀H₂₁NO₃: 323, found [M+H]⁺=324.

Synthesis of chiral 7-((1-methoxypropan-2-yl)oxy)-4-(o-tolyl)isoquinolin-1(2H)-one, Examples 26 and 27 [Step 3]: 7-((1-methoxypropan-2-yl)oxy)-4-(o-tolyl)isoquinolin-1(2H)-one (Example 25, 90 mg, 0.3 mmol) was purified by normal phase chiral prep-HPLC and lyophilized. The first product was isolated as 7-((1-methoxypropan-2-yl)oxy)-4-(o-tolyl)isoquinolin-1(2H)-one, Peak 1 (Example 26, 30 mg) and the second product as 7-((1-methoxypropan-2-yl)oxy)-4-(o-tolyl)isoquinolin-1(2H)-one, Peak 2 (Example 27, 30 mg). The absolute stereochemistry for these Examples was not determined.

Example 26: 7-((1-methoxypropan-2-yl)oxy)-4-(o-tolyl)isoquinolin-1(2H)-one, Peak 1: LCMS (ESI) Calcd. for C₂₀H₂₁NO₃: 323, found [M+H]⁺=324. ¹H NMR (400 MHz, DMSO-d₆) δ 11.32 (d, 1H), 7.71 (d, 1H), 7.34 (d, 2H), 7.30-7.24 (m, 2H), 7.20 (d, 1H), 6.91-6.86 (m, 2H), 4.74-4.70 (m, 1H), 3.55-3.46 (m, 2H), 3.31-3.26 (m, 3H), 2.04 (s, 3H), 1.27 (d, 3H).

Example 27: 7-((1-methoxypropan-2-yl)oxy)-4-(o-tolyl)isoquinolin-1(2H)-one, Peak 2: LCMS (ESI) Calcd. for C₂₀H₂₁NO₃: 323, found [M+H]⁺=324. ¹H NMR (400 MHz, DMSO-d₆) δ 11.33 (d, 1H), 7.71 (d, 1H), 7.34 (d, 2H), 7.30-7.24 (m, 2H), 7.20 (d, 1H), 6.91-6.86 (m, 2H), 4.74-4.70 (m, 1H), 3.55-3.46 (m, 2H), 3.30-3.28 (m, 3H), 2.04 (s, 3H), 1.27 (d, 3H).

Chiral prep-HPLC: Chiral separation was performed on an Agilent 1200 series instrument. Column was a Chiralpak IC (250×21 mm), 5 μm, operating at ambient temperature with flow rate of 18 mL/min. Mobile phase: 85% hexane, 7.5% EtOAc, and 7.5% ethanol, held isocratic for up to 14 min. with detection at 282 nm wavelength.

### Example 28: Synthesis of (S)—N-methyl-2-((1-oxo-4-(o-tolyl)-1,2-dihydroisoquinolin-7-yl)oxy)propenamide

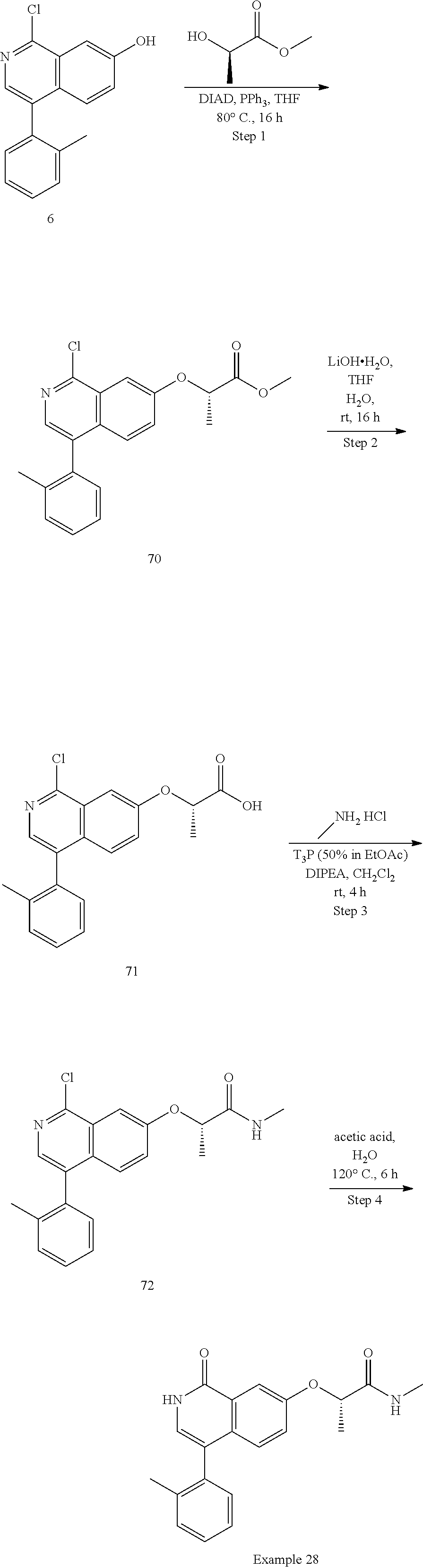

Synthesis of methyl (S)-2-((1-chloro-4-(o-tolyl)isoquinolin-7-yl)oxy)propanoate, 70 [Step 1]: To a stirred solution of 1-chloro-4-(o-tolyl)isoquinolin-7-ol (6, 120 mg, 0.4 mmol) in THF (5 mL) was added PPh₃ (350 mg, 1.3 mmol) followed by DIAD (0.3 mL, 1.3 mmol). The reaction mixture was heated at 80° C. for 16 h. The reaction mixture was concentrated under reduced pressure. The reaction mixture was partitioned between EtOAc and water. The organic layer was collected, washed with brine, dried over anhydrous Na₂SO₄, filtered, and concentrated under reduced pressure. The product was purified by flash chromatography to afford methyl (S)-2-((1-chloro-4-(o-tolyl)isoquinolin-7-yl)oxy)propanoate (70, 130 mg). LCMS (ESI) Calcd. for C₂₀H₁₈ClNO₃: 355, found [M+H]⁺=356. ¹H NMR (400 MHz, DMSO-d₆) δ 8.06 (d, 1H), 7.55 (d, 1H), 7.52 (d, 1H), 7.45-7.42 (m, 2H), 7.36-7.34 (m, 2H), 7.26-7.23 (t, 1H), 5.28-5.23 (m, 1H), 3.74-3.73 (d, 3H), 1.99-1.94 (d, 3H), 1.61-1.59 (d, 3H).

Synthesis of (S)-2-((1-chloro-4-(o-tolyl)isoquinolin-7-yl)oxy)propanoic acid, 71 [Step 2]: To a stirred solution of methyl (S)-2-((1-chloro-4-(o-tolyl)isoquinolin-7-yl)oxy)propanoate (70, 130 mg, 0.4 mmol) in THF (3 mL) and water (1 mL) was added LiOH·H₂O (62 mg, 1.5 mmol). The reaction mixture was stirred at ambient temperature for 2 h. The reaction mixture was diluted with water, acidified with 10% aq. citric acid solution, and extracted with EtOAc. The combined organic layers were washed with brine, dried over anhydrous Na₂SO₄, filtered, and concentrated under reduced pressure to afford (S)-2-((1-chloro-4-(o-tolyl)isoquinolin-7-yl)oxy)propanoic acid (71, 120 mg). LCMS (ESI) Calcd. for C₁₉H₁₆ClNO₃: 341, found [M+H]⁺=342.

Synthesis of (S)-2-((1-chloro-4-(o-tolyl)isoquinolin-7-yl)oxy)-N-methylpropanamide, 72 [Step 3]: To a stirred solution of (S)-2-((1-chloro-4-(o-tolyl)isoquinolin-7-yl)oxy)propanoic acid (71, 145 mg, 0.5 mmol) in CH₂Cl₂ (5 mL) was added DIPEA (0.2 mL, 1.3 mmol) followed by methylamine hydrochloride (290 mg, 4.3 mmol). T₃P (0.4 mL, 0.6 mmol, 50% in EtOAc) was added at 0° C. The reaction mixture was stirred at ambient temperature for 4 h. The reaction mixture was concentrated under reduced pressure. The product was partitioned between EtOAc and water. The organic layer was collected, washed with brine, dried over anhydrous Na₂SO₄, filtered, and concentrated under reduced pressure. The product was purified by flash chromatography to afford (S)-2-((1-chloro-4-(o-tolyl)isoquinolin-7-yl)oxy)-N-methylpropanamide (72, 130 mg). LCMS (ESI) Calcd. for C₂₀H₁₉ClN₂O₂: 354, found [M+H]⁺=355.

Synthesis of (S)—N-methyl-2-((1-oxo-4-(o-tolyl)-1,2-dihydroisoquinolin-7-yl)oxy)propenamide, Example 28 [Step 4]: To a stirred solution of (S)-2-((1-chloro-4-(o-tolyl)isoquinolin-7-yl)oxy)-N-methylpropanamide (72, 210 mg, 0.6 mmol) in acetic acid (5.1 mL, 89 mmol) was added water (1.1 mL, 59.2 mmol) at ambient temperature. The reaction mixture was heated to reflux for 16 h. The reaction mixture was concentrated under reduced pressure. The reaction mixture was partitioned between EtOAc and water. The organic phase was collected, washed with brine, dried over anhydrous Na₂SO₄, filtered, and concentrated under reduced pressure. The product was further purified by reverse phase prep-HPLC and lyophilized to afford (S)—N-methyl-2-((1-oxo-4-(o-tolyl)-1,2-dihydroisoquinolin-7-yl)oxy)propenamide (Example 28, 70 mg). LCMS (ESI) Calcd. for C₂₀H₂₀N₂O₃: 336, found [M+H]⁺=337. ¹H NMR (400 MHz, DMSO-d₆) δ 11.34 (s, 1H), 8.14-8.10 (m, 1H), 7.65 (d, 1H), 7.35 (d, 2H), 7.31-7.26 (m, 2H), 7.19 (d, 1H), 6.93-6.88 (m, 2H), 4.82 (d, 1H), 2.66 (s, 3H), 2.04 (d, 3H), 1.47 (d, 3H).

### Examples 29-33: Synthesis of 7-amino-4-(o-tolyl)isoquinolin-1(2H)-one, N-(1-oxo-4-(o-tolyl)-1,2-dihydroisoquinolin-7-yl)isobutyramide, N-(1-oxo-4-(o-tolyl)-1,2-dihydroisoquinolin-7-yl)acetamide, 2-methoxy-N-(1-oxo-4-(o-tolyl)-1,2-dihydroisoquinolin-7-yl)acetamide, and 1-acetyl-N-(1-oxo-4-(o-tolyl)-1,2-dihydroisoquinolin-7-yl) piperidine-4-carboxamide

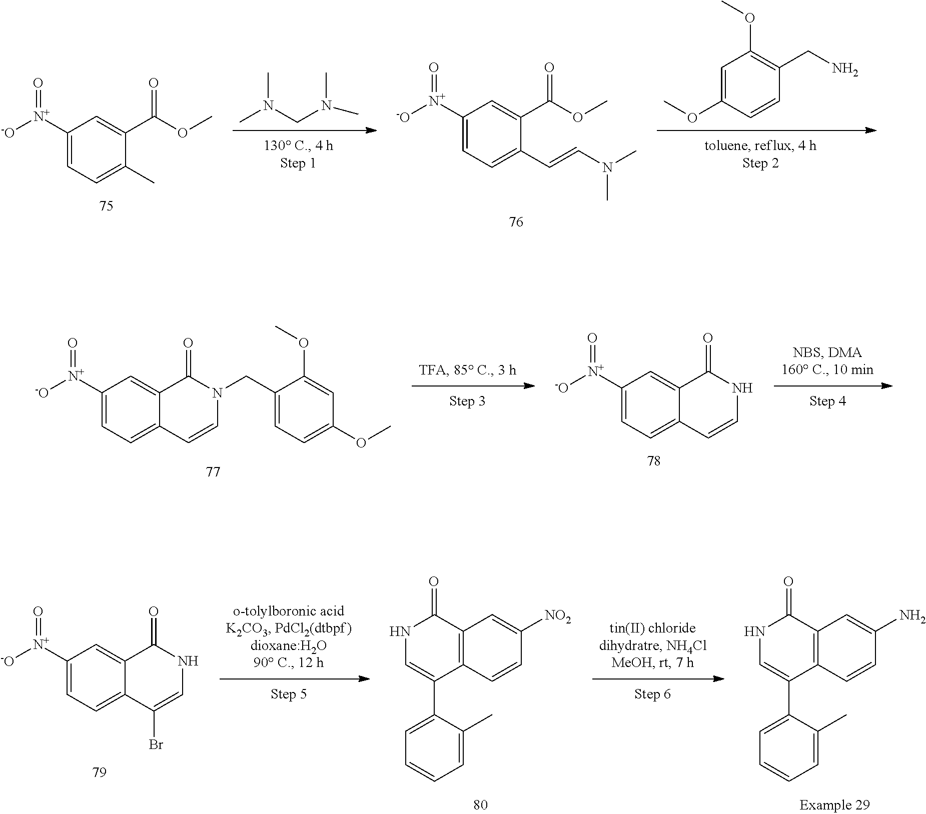

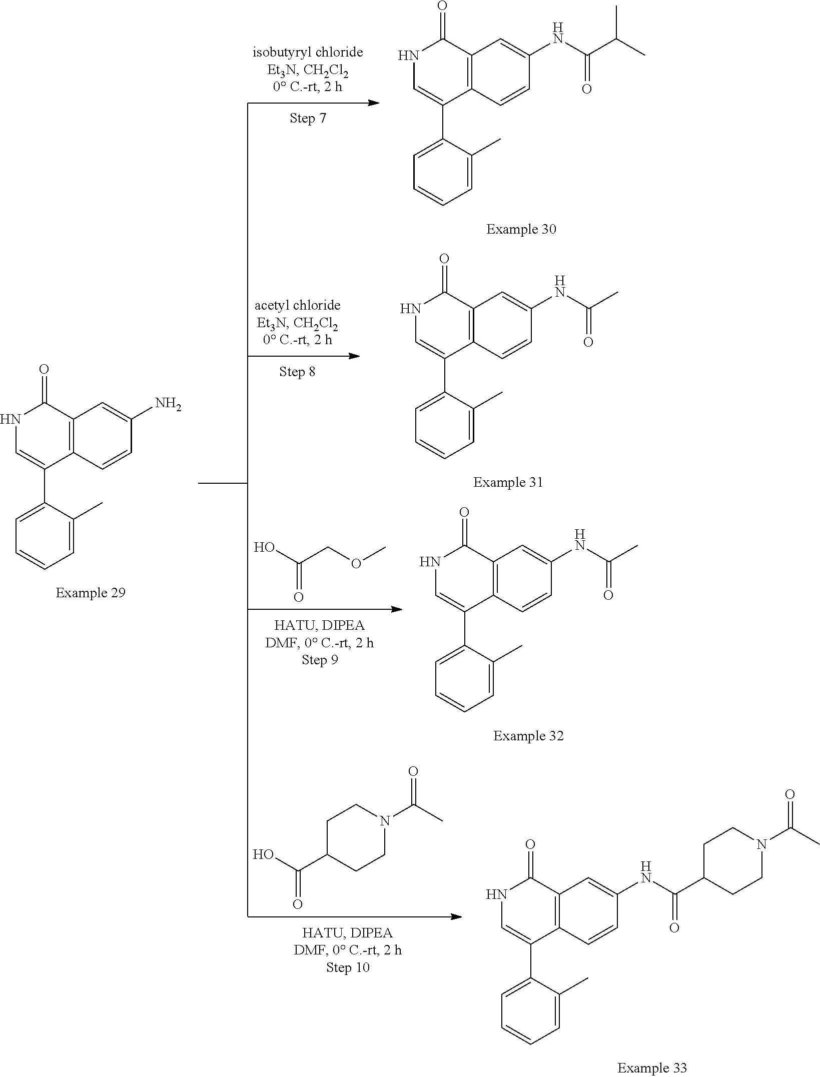

Synthesis of methyl (E)-2-(2-(dimethylamino)vinyl)-5-nitrobenzoate, 76 [Step 1]: A mixture of methyl 2-methyl-5-nitrobenzoate (75, 20 g, 102.5 mmol) and N,N,N′,N′-tetramethylmethanediamine (41 g, 235 mmol) was heated at 115° C. for 2 h under an argon atmosphere. The reaction mixture was cooled to ambient temperature and triturated with EtOAc/hexanes (6:1). The reaction mixture was filtered to afford methyl (E)-2-(2-(dimethylamino)vinyl)-5-nitrobenzoate (76, 24 g). ¹H NMR (400 MHz, DMSO-d₆) δ 8.89 (d, 1H), 8.03 (dd, 1H), 7.42 (t, 1H), 7.17 (d, 1H), 6.40 (d, 1H), 3.88 (d, 3H), 2.99 (s, 6H).

Synthesis of 2-(2,4-dimethoxybenzyl)-7-nitroisoquinolin-1(2H)-one, 77 [Step 2]: To a stirred solution of methyl (E)-2-(2-(dimethylamino)vinyl)-5-nitrobenzoate (76, 24 g, 95 mmol) in toluene (250 mL) was added 2,4-dimethoxyphenyl-methanamine (22 g, 134 mmol) at ambient temperature. The reaction mixture was heated to 125° C. for 3.5 h. The reaction mixture was cooled to ambient temperature and triturated with EtOAc/hexanes (1:2) to afford 2-(2,4-dimethoxybenzyl)-7-nitroisoquinolin-1(2H)-one (77, 24 g). ¹H NMR (400 MHz, DMSO-d₆) δ 9.28 (s, 1H), 8.36 (d, 1H), 7.57 (d, 1H), 7.41 (dd, 2H), 6.47 (d, 3H), 5.12 (s, 2H), 3.80 (d, 6H).

Synthesis of 7-nitroisoquinolin-1(2H)-one, 78 [Step 3]: A solution of 2-(2,4-dimethoxybenzyl)-7-nitroisoquinolin-1(2H)-one (77, 20 g, 58 mmol) in TFA (80 mL) was heated at 85° C. for 2.5 h. The reaction mixture was cooled to ambient temperature. The reaction mixture was concentrated under vacuum. The product was further triturated with EtOAc and collected by filtration to afford 7-nitroisoquinolin-1(2H)-one (78, 20 g). LCMS (ESI) Calcd. for C₉H₆N₂O₃: 190, found [M+H]⁺=191. ¹H NMR (400 MHz, DMSO-d₆) δ 11.76 (s, 1H), 8.89 (s, 1H), 8.44 (dd, 1H), 7.90 (d, 1H), 7.45 (t, 1H), 6.73 (d, 1H).

Synthesis of 4-bromo-7-nitroisoquinolin-1(2H)-one, 79 [Step 4]: To a stirred solution of 7-nitroisoquinolin-1(2H)-one (78, 5 g, 26 mmol) in DMA (50 mL) was added NBS (4.67 g, 26.3 mmol) under argon atmosphere. The reaction mixture was stirred for 10 min., quenched with water, filtered, washed with water, and dried under vacuum to afford 4-bromo-7-nitroisoquinolin-1(2H)-one (79, 4 g). ¹H NMR (400 MHz, DMSO-d₆) δ 12.10 (s, 1H), 8.90 (s, 1H), 8.58 (d, 1H), 7.98 (dd, 1H), 7.86 (dd, 1H).

Synthesis of 7-nitro-4-(o-tolyl)isoquinolin-1(2H)-one, 80 [Step 5]: An oven-dried sealed tube was charged with 4-bromo-7-nitroisoquinolin-1(2H)-one (79, 2 g, 7.43 mmol) followed by o-tolyl boronic acid and K₂CO₃ (3.1 g, 22.3 mmol). To the charged tube was added 1,4-dioxane and water (4:1). The reaction mixture was degassed with nitrogen for 10 min. PdCl₂(dtbpf) (480 mg, 0.7 mmol) was added, and the reaction mixture was heated at 90° C. for 12 h. The reaction mixture was cooled to ambient temperature and partitioned between EtOAc and water. The organic layer was collected, washed with brine, dried over anhydrous Na₂SO₄, and concentrated under reduced pressure. The product was purified by column chromatography to afford 7-nitro-4-(o-tolyl)isoquinolin-1(2H)-one (80, 1 g). LCMS (ESI) Calcd. for C₁₆H₁₂N₂O₃: 280, found [M+H]⁺=281. ¹H NMR (400 MHz, DMSO-d₆) δ 11.94 (s, 1H), 8.98 (d, 1H), 8.40 (dd, 1H), 7.38 (d, 2H), 7.31 (t, 2H), 7.24 (d, 1H), 7.17 (d, 1H), 2.01 (d, 3H).

Synthesis of 7-amino-4-(o-tolyl)isoquinolin-1(2H)-one, Example 29 [Step 6]: To a stirred solution of 7-nitro-4-(o-tolyl)isoquinolin-1(2H)-one (80, 1 g, 3.6 mmol) in MeOH (10 mL) was added tin(II) chloride dihydrate (2.7 g, 14.3 mmol) and ammonium chloride (954 mg, 17.8 mmol) under an argon atmosphere. The reaction mixture was stirred at ambient temperature for 7 h. The reaction mixture was concentrated under reduced pressure and partitioned between EtOAc and a saturated NH₄Cl solution. The combined organic layers were washed with brine (×2), dried over anhydrous Na₂SO₄, and concentrated under reduced pressure to afford 7-amino-4-(o-tolyl)isoquinolin-1(2H)-one (Example 29, 700 mg). LCMS (ESI) Calcd. for C₁₆H₁₄N₂O: 250, found [M+H]⁺=251.

Synthesis of N-(1-oxo-4-(o-tolyl)-1,2-dihydroisoquinolin-7-yl)isobutyramide, Example 30 [Step 7]: To a stirred solution of 7-amino-4-(o-tolyl)isoquinolin-1(2H)-one (Example 29, 100 mg, 0.4 mmol) in CH₂Cl₂ (3 mL) was added triethylamine (0.1 mL, 0.8 mmol), followed by isobutyryl chloride (53 mg, 0.5 mmol) at 0° C. The reaction mixture was gradually warmed to ambient temperature and stirred for 2 h. The reaction mixture was diluted with CH₂Cl₂, washed with water (×2) and brine, dried over anhydrous Na₂SO₄, filtered, and concentrated under reduced pressure. The product was purified by column chromatography to afford N-(1-oxo-4-(o-tolyl)-1,2-dihydroisoquinolin-7-yl)isobutyramide (Example 30, 40 mg). LCMS (ESI) Calcd. for C₂₀H₂₀N₂O₂: 320, found [M+H]⁺=321. ¹H NMR (400 MHz, DMSO-d₆) δ 11.29 (d, 1H), 10.11 (s, 1H), 8.58 (d, 1H), 7.82 (dd, 1H), 7.34 (d, 2H), 7.28 (t, 1H), 7.20 (d, 1H), 6.89 (t, 2H), 2.60 (t, 1H), 2.03 (s, 3H), 1.10 (d, 6H).

Synthesis of N-(1-oxo-4-(o-tolyl)-1,2-dihydroisoquinolin-7-yl)acetamide, Example 31 [Step 8]: To a stirred solution of 7-amino-4-(o-tolyl)isoquinolin-1(2H)-one (Example 29, 100 mg, 0.4 mmol) in CH₂Cl₂ (3 mL) was added triethylamine (0.1 mL, 0.8 mmol) followed by acetyl chloride (0.034 mL, 0.5 mmol) at 0° C. The reaction mixture was gradually warmed to ambient temperature and stirred for 2 h. The reaction mixture was diluted with CH₂Cl₂, washed with water (×2) and brine, dried over anhydrous Na₂SO₄, filtered, and concentrated under reduced pressure. The product was purified by column chromatography to afford N-(1-oxo-4-(o-tolyl)-1,2-dihydroisoquinolin-7-yl)acetamide (Example 31, 32 mg). LCMS (ESI) Calcd. for C₁₈H₁₆N₂O₂: 292, found [M+H]⁺=293. ¹H NMR (400 MHz, DMSO-d₆) δ 11.28 (s, 1H), 10.21 (s, 1H), 8.57 (s, 1H), 7.78 (br s, 1H), 7.35-7.19 (m, 4H), 6.93-6.88 (m, 2H), 2.05 (s, 6H).

Synthesis of 2-methoxy-N-(1-oxo-4-(o-tolyl)-1,2-dihydroisoquinolin-7-yl)acetamide, Example 32 [Step 9]: To a solution of 2-methoxy acetic acid (43 mg, 0.47 mmol) in DMF (4 ml) was added DIPEA (80 mg, 0.8 mmol) and HATU (151 mg, 0.4 mmol). The reaction mixture was stirred for 15 min. at ambient temperature, and 7-amino-4-(o-tolyl)-2H-isoquinolin-1-one (Example 29, 100 mg, 0.4 mmol) was added. The reaction mixture was stirred at ambient temperature for 2 h. The reaction mixture was diluted with cold water and extracted with EtOAc. The combined organic layers were washed with cold water, brine, dried over anhydrous Na₂SO₄, filtered, and concentrated under reduced pressure. The product was purified by reverse phase prep-HPLC and lyophilized to afford 2-methoxy-N-(1-oxo-4-(o-tolyl)-1,2-dihydroisoquinolin-7-yl)acetamide (Example 32, 30 mg). LCMS (ESI) Calcd. for C₁₉H₁₈N₂O₃: 322, found [M+H]⁺=323. ¹H NMR (400 MHz, DMSO-d₆) δ 11.32 (d, 1H), 10.07 (s, 1H), 8.67 (d, 1H), 7.85 (dd, 1H), 7.35 (d, 2H), 7.31-7.26 (m, 1H), 7.20 (d, 1H), 6.94-6.91 (m, 2H), 4.03 (s, 2H), 3.38 (s, 3H), 2.07 (s, 3H).

Synthesis of 1-acetyl-N-(1-oxo-4-(o-tolyl)-1,2-dihydroisoquinolin-7-yl) piperidine-4-carboxamide, Example 33 [Step 10]: To a solution of 1-acetylpiperidine-4-carboxylic acid (82 gm, 0.5 mmol) in DMF (4 mL) was added DIPEA (80 mg, 0.8 mmol) and HATU (151 mg, 0.4 mmol). The reaction mixture was stirred for 15 min. at ambient temperature. To the reaction mixture was added 7-amino-4-(o-tolyl)-2H-isoquinolin-1-one (Example 29, 100 mg, 0.4 mmol). The reaction mixture was stirred at ambient temperature for 2 h. The reaction mixture was diluted with cold water and extracted with EtOAc. The combined organic layers were washed with cold water, brine, dried over anhydrous Na₂SO₄, filtered, and concentrated under reduced pressure. The product was purified by reverse phase prep-HPLC and lyophilized to afford 1-acetyl-N-(1-oxo-4-(o-tolyl)-1,2-dihydroisoquinolin-7-yl)piperidine-4-carboxamide (Example 33, 40 mg). LCMS (ESI) Calcd. for C₂₄H₂₅N₃O₃: 403, found [M+H]⁺=404. ¹H NMR (400 MHz, DMSO-d₆) at 100° C. δ 10.93 (br s, 1H), 9.86 (s, 1H), 8.50 (s, 1H), 7.84 (d, 1H), 7.34 (d, 2H), 7.30-7.26 (m, 1H), 7.19 (d, 1H), 6.93-6.86 (m, 2H), 4.25 (m, 2H), 3.53-3.46 (m, 2H), 2.65 (m, 2H), 2.04 (s, 3H), 2.01 (s, 3H), 1.82 (m, 2H), 1.68 (m, 2H).

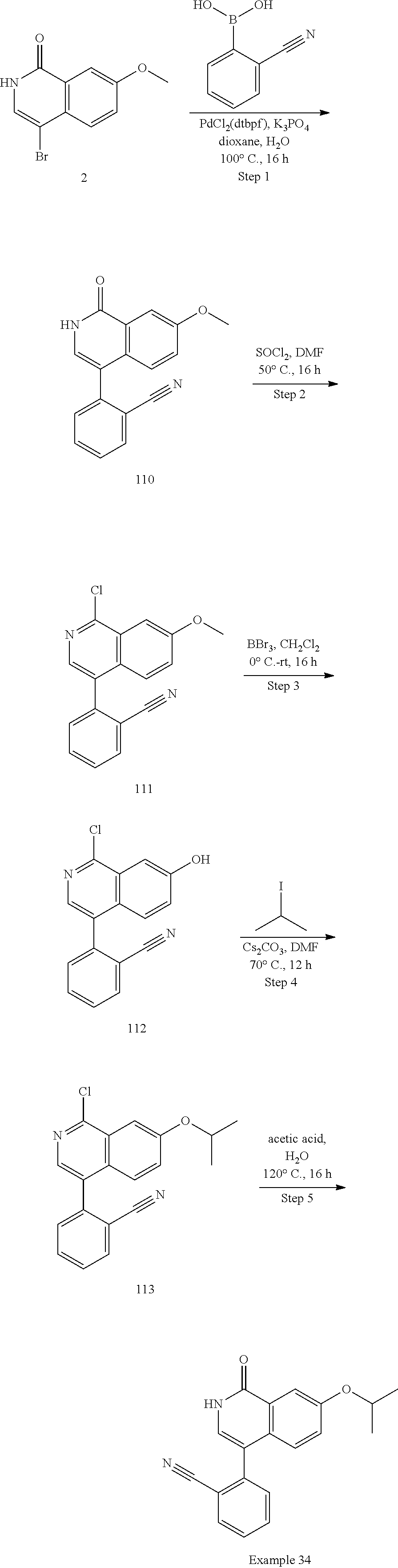

### Example 34: Synthesis of 2-(7-isopropoxy-1-oxo-1,2-dihydroisoquinolin-4-yl)benzonitrile

Synthesis of 2-(7-methoxy-1-oxo-1,2-dihydroisoquinolin-4-yl)benzonitrile, 110 [Step 1]: To a stirred solution of 4-bromo-7-methoxy-2H-isoquinolin-1-one (2, 1.0 g, 4.0 mmol) in 1,4-dioxane (24 mL) and water (8 mL) was added K₃PO₄ (2.1 g, 10 mmol). The reaction mixture was degassed with argon. (2-cyanophenyl)boronic acid (865 mg, 6.0 mmol) and PdCl₂(dtbpf) (255 mg, 0.4 mmol) were added, and the reaction mixture was stirred at 100° C. for 16 h. The reaction mixture was filtered through celite. The filtrate was diluted with EtOAc, washed with water, brine, dried over anhydrous Na₂SO₄, filtered, and concentrated under reduced pressure. The product was purified by flash column chromatography to afford 2-(7-methoxy-1-oxo-1,2-dihydroisoquinolin-4-yl)benzonitrile (110, 550 mg). LCMS (ESI) Calcd. for C₁₇H₁₂N₂O₂: 276, found [M+H]+=277. ¹H NMR (400 MHz, DMSO-d₆) δ 11.58 (s, 1H), 7.99 (d, 1H), 7.81 (t, 1H), 7.73 (d, 1H), 7.64 (t, 1H), 7.62-7.59 (m, 1H), 7.32 (dd, 1H), 7.16 (d, 1H), 7.10 (d, 1H), 3.31 (s, 3H).

Synthesis of 2-(1-chloro-7-methoxyisoquinolin-4-yl)benzonitrile, 111 [Step 2]: To a stirred solution of 2-(7-methoxy-1-oxo-1,2-dihydroisoquinolin-4-yl)benzonitrile (110, 550 mg, 2.0 mmol) in SOCl₂ (7.2 mL, 100 mmol) was added DMF (0.2 mL, 2.0 mmol) under argon atmosphere at ambient temperature. The reaction mixture was heated at 50° C. for 16 h. The reaction mixture was concentrated under reduced pressure, quenched with ice water, and extracted with EtOAc. The combined organic layers were washed with water, brine, dried over Na₂SO₄, filtered, and concentrated under reduced pressure. The product was purified by flash column chromatography to afford 2-(1-chloro-7-methoxyisoquinolin-4-yl)benzonitrile (111, 340 mg). LCMS (ESI) Calcd. for C₁₇HnClN₂O: 294, found [M+H]⁺=295. ¹H NMR (400 MHz, DMSO-d₆) δ 8.22 (s, 1H), 8.08 (d, 1H), 7.90 (t, 1H), 7.75 (t, 1H), 7.72-7.62 (m, 2H), 7.61-7.52 (m, 1H), 7.49 (d, 1H), 4.00 (s, 3H).

Synthesis of 2-(1-chloro-7-hydroxyisoquinolin-4-yl)benzonitrile, 112 [Step 3]: To a stirred solution 2-(1-chloro-7-methoxy-4-isoquinolyl)benzonitrile (111, 340 mg, 1.0 mmol) in CH₂Cl₂ (4 mL) was added dropwise BBr₃ (3.4 ml, 3.4 mmol, 1M in CH₂Cl₂) at 0° C. The reaction mixture was warmed to ambient temperature and stirred for 16 h. The reaction mixture was cooled to 0° C. and quenched with MeOH (1 mL). The reaction mixture was concentrated under reduced pressure and partitioned between EtOAc and water. The organic layer was collected, washed with brine, dried over anhydrous Na₂SO₄, filtered, and concentrated to afford the product. The product was purified by flash column chromatography to afford 2-(1-chloro-7-hydroxyisoquinolin-4-yl)benzonitrile (112, 300 mg). LCMS (ESI) Calcd. for C₁₆H₉ClN₂O: 280, found [M+H]+=281. ¹H NMR (400 MHz, DMSO-d₆) δ 10.74 (s, 1H), 8.12 (s, 1H), 8.07 (d, 1H), 7.90 (t, 1H), 7.76 (t, 1H), 7.68 (d, 1H), 7.61 (s, 1H), 7.43-7.41 (m, 2H).

Synthesis of 2-(1-chloro-7-isopropoxyisoquinolin-4-yl)benzonitrile, 113 [Step 4]: To a stirred solution of 2-(1-chloro-7-hydroxyisoquinolin-4-yl)benzonitrile (112, 100 mg, 0.4 mmol) in DMF (2 mL) was added Cs₂CO₃ (230 mg, 0.8 mmol) followed by 2-iodopropane (0.1 mL, 0.8 mmol). The reaction mixture was heated at 70° C. for 12 h. The reaction mixture was quenched with water and extracted with EtOAc (×3). The combined organic layers were washed with brine, dried over anhydrous Na₂SO₄, filtered, and concentrated to afford 2-(1-chloro-7-isopropoxyisoquinolin-4-yl)benzonitrile (113, 110 mg). LCMS (ESI) Calcd. for C₁₉H₁₅ClN₂O: 322, found [M+H]⁺=323. ¹H NMR (400 MHz, DMSO-d₆) δ 8.20 (s, 1H), 8.09 (d, 1H), 7.90 (t, 1H), 7.75 (t, 1H), 7.69 (d, 1H), 7.64 (d, 1H), 7.54 (dd, 1H), 7.47 (d, 1H), 4.94 (m, 1H), 1.39 (d, 6H).

Synthesis of 2-(7-isopropoxy-1-oxo-1,2-dihydroisoquinolin-4-yl)benzonitrile, Example 34 [Step 5]: To a stirred solution of 2-(1-chloro-7-isopropoxyisoquinolin-4-yl)benzonitrile (113, 150 mg, 0.5 mmol) in acetic acid (3.1 mL, 53.4 mmol) was added water (0.9 mL). The reaction mixture was stirred at 120° C. for 16 h. The reaction mixture was concentrated under reduced pressure. The reaction mixture was diluted with EtOAc, washed with water, brine, dried over anhydrous Na₂SO₄, filtered, and concentrated under reduced pressure. The product was purified via reverse phase prep-HPLC and lyophilized to afford 2-(7-isopropoxy-1-oxo-1,2-dihydroisoquinolin-4-yl)benzonitrile (Example 34, 45 mg). LCMS (ESI) Calcd. for C₁₉H₁₆N₂O₂: 304, found [M+H]⁺=305. ¹H NMR (400 MHz, DMSO-d₆) δ 11.55 (s, 1H), 7.99 (d, 1H), 7.81 (t, 1H), 7.71 (d, 1H), 7.66-7.59 (m, 2H), 7.28 (dd, 1H), 7.15 (s, 1H), 7.09 (d, 1H), 4.78-4.72 (m, 1H), 1.32 (d, 6H).

### Example 35: Synthesis of (S)-1-((R)-2-((1-oxo-4-(o-tolyl)-1,2-dihydroisoquinolin-7-yl)oxy)propanoyl)pyrrolidine-3-carboxylic acid

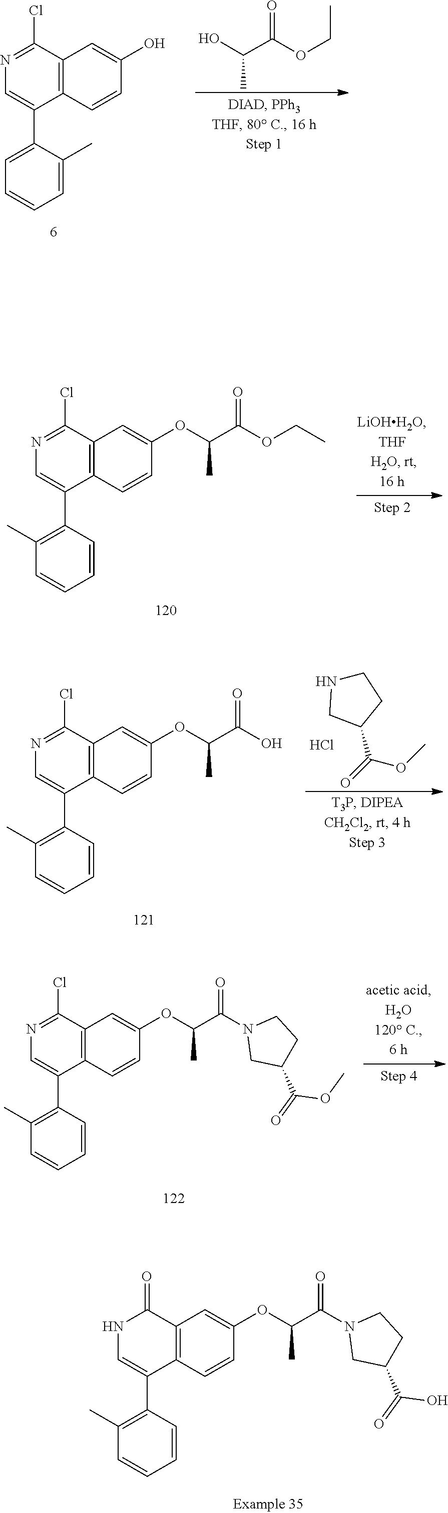

Synthesis of ethyl (R)-2-((1-chloro-4-(o-tolyl)isoquinolin-7-yl)oxy)propanoate, 120 [Step 1]: To a stirred solution of 1-chloro-4-(o-tolyl)isoquinolin-7-ol (6, 220 mg, 0.8 mmol) in THF (5 mL) was added PPh₃ (640 mg, 2.4 mmol) followed by DIAD (0.5 mL, 2.4 mmol). The reaction mixture was heated at 80° C. for 16 h. The reaction mixture was concentrated under reduced pressure and partitioned between EtOAc and water. The organic phase was washed with brine, dried over anhydrous Na₂SO₄, filtered, and concentrated under reduced pressure. The product was purified by flash chromatography to afford ethyl (R)-2-((1-chloro-4-(o-tolyl)isoquinolin-7-yl)oxy)propanoate (120, 300 mg). LCMS (ESI) Calcd. for C₂₁H₂₀ClNO₃: 369, found [M+H]⁺=370. ¹H NMR (400 MHz, DMSO-d₆) δ 8.06 (d, 1H), 7.55 (d, 1H), 7.50 (d, 1H), 7.45-7.42 (m, 2H), 7.37-7.33 (m, 2H), 7.26-7.24 (t, 1H), 5.27-5.15 (m, 1H), 4.25-4.15 (m, 2H), 1.97-1.89 (dd, 3H), 1.60 (d, 3H), 1.25-1.17 (m, 3H).

Synthesis of (R)-2-((1-chloro-4-(o-tolyl)isoquinolin-7-yl)oxy)propanoic acid, 121 [Step 2]: To a stirred solution of ethyl (R)-2-((1-chloro-4-(o-tolyl)isoquinolin-7-yl)oxy)propanoate (120, 300 mg, 0.8 mmol) in THF (3 mL) and water (1 mL) was added LiOH·H₂O (140 mg, 3.2 mmol). The reaction mixture was stirred for 2 h at ambient temperature. The reaction mixture was diluted with water, acidified with 10% aq. citric acid solution, and extracted with EtOAc. The combined organic layers were washed with brine, dried over anhydrous Na₂SO₄, filtered, and concentrated under reduced pressure to afford (R)-2-((1-chloro-4-(o-tolyl)isoquinolin-7-yl)oxy)propanoic acid (121, 220 mg). LCMS (ESI) Calcd. for C₁₉H₁₆ClNO₃: 341, found [M+H]⁺=342.

Synthesis of methyl (S)-1-((R)-2-((1-chloro-4-(o-tolyl)isoquinolin-7-yl)oxy)propanoyl)pyrrolidine-3-carboxylate, 122 [Step 3]: To a stirred solution of (R)-2-((1-chloro-4-(o-tolyl)isoquinolin-7-yl)oxy)propanoic acid (121, 140 mg, 0.4 mmol) in CH₂Cl₂ (5 mL) was added DIPEA (0.2 mL, 1.2 mmol), methyl (S)-pyrrolidine-3-carboxylate hydrochloride (205 mg, 1.23 mmol), and T₃P (0.2 mL, 0.6 mmol, 50% in EtOAc) at 0° C. The reaction mixture was stirred at ambient temperature for 4 h. The reaction mixture was concentrated under reduced pressure. The product was partitioned between EtOAc and water. The organic phase was washed with brine, dried over anhydrous Na₂SO₄, filtered, and concentrated under reduced pressure. The product was purified by flash column chromatography to afford methyl (S)-1-((R)-2-((1-chloro-4-(o-tolyl)isoquinolin-7-yl)oxy)propanoyl)pyrrolidine-3-carboxylate (122, 120 mg). LCMS (ESI) Calcd. for C₂₅H₂₅ClN₂O₄: 452, found [M+H]⁺=453.

Synthesis of (S)-1-((R)-2-((1-oxo-4-(o-tolyl)-1,2-dihydroisoquinolin-7-yl)oxy)propanoyl)pyrrolidine-3-carboxylic acid, Example 35 [Step 4]: To a stirred solution of methyl (S)-1-((R)-2-((1-chloro-4-(o-tolyl)isoquinolin-7-yl)oxy)propanoyl)pyrrolidine-3-carboxylate (122, 120 mg, 0.3 mmol) in acetic acid (2.2 mL, 38.5 mmol) was added water (0.5 mL, 26 mmol) at ambient temperature. The reaction mixture was heated to reflux for 16 h. The reaction mixture was concentrated under reduced pressure and partitioned between EtOAc and water. The organic phase was washed with brine, dried over anhydrous Na₂SO₄, filtered, and concentrated under reduced pressure. The product was purified by reverse phase prep-HPLC and lyophilized to afford (S)-1-((R)-2-((1-oxo-4-(o-tolyl)-1,2-dihydroisoquinolin-7-yl)oxy)propanoyl)pyrrolidine-3-carboxylic acid (Example 35, 40 mg). LCMS (ESI) Calcd. for C₂₄H₂₄N₂O₅: 420, found [M+H]⁺=421. ¹H NMR (400 MHz, DMSO-d₆) δ 11.34 (d, 1H), 7.62-7.57 (m, 1H), 7.35 (d, 2H), 7.30-7.18 (m, 3H), 6.91-6.88 (m, 2H), 5.16 (d, 1H), 3.57-3.55 (m, 1H), 3.54-3.52 (m, 2H), 3.47-3.43 (m, 1H), 3.32-3.27 (m, 1H), 2.50-2.49 (m, 1H). 2.03 (m, 5H), 1.46-1.43 (m, 3H).

### Example 36: Synthesis of 2-((1-oxo-4-(o-tolyl)-1,2-dihydroisoquinolin-7-yl)oxy)acetonitrile

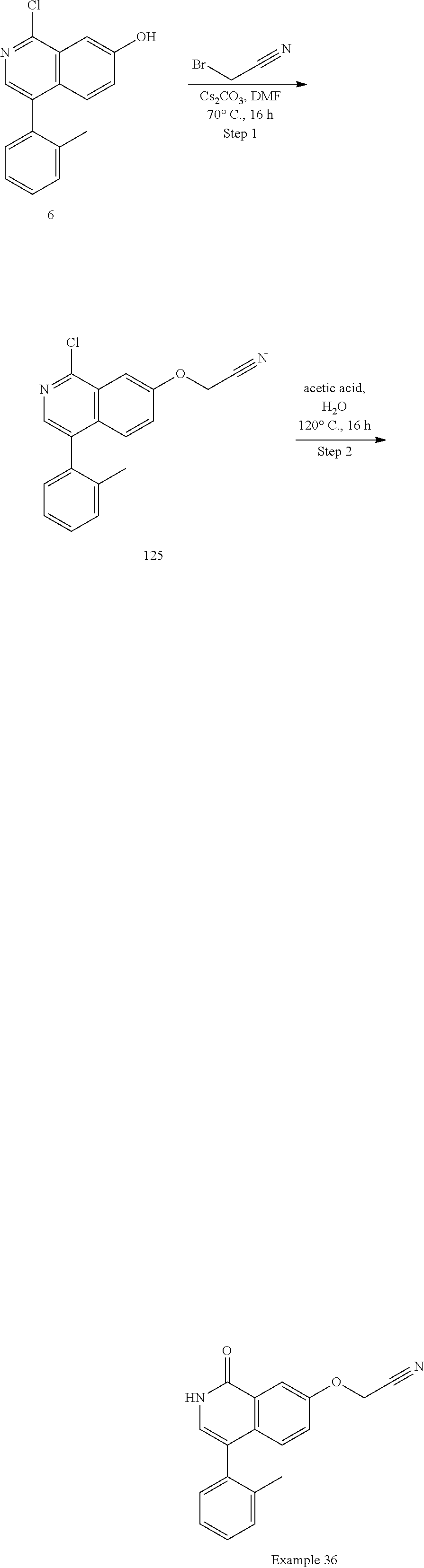

Synthesis of 2-((1-chloro-4-(o-tolyl)isoquinolin-7-yl)oxy)acetonitrile, 125 [Step 1]: To a stirred solution of 1-chloro-4-(o-tolyl)isoquinolin-7-ol (6, 150 mg, 0.6 mmol) in DMF (2 mL) was added Cs₂CO₃ (362 mg, 1.1 mmol) followed by 2-bromoacetonitrile (0.08 mL, 1.1 mmol). The reaction mixture was heated at 70° C. for 12 h. The reaction mixture was quenched with water and extracted with EtOAc (2×25 mL). The combined organic layers were washed with brine, dried over anhydrous Na₂SO₄, filtered, and concentrated under reduced pressure. The product was purified by flash chromatography to afford 2-((1-chloro-4-(o-tolyl)isoquinolin-7-yl)oxy)acetonitrile (125, 160 mg). LCMS (ESI) Calcd. for C₁₈H₁₃ClN₂O: 308, found [M+H]⁺=309. ¹H NMR (400 MHz, DMSO-d₆) δ 8.12 (s, 1H), 7.80 (d, 1H), 7.61-7.58 (dd, 1H), 7.47-7.34 (m, 4H), 7.27-7.25 (d, 1H), 5.47 (s, 2H), 1.98 (s, 3H).

Synthesis of 2-((1-oxo-4-(o-tolyl)-1,2-dihydroisoquinolin-7-yl)oxy)acetonitrile, Example 36 [Step 2]: To a stirred solution of 2-((1-chloro-4-(o-tolyl)isoquinolin-7-yl)oxy)acetonitrile (125, 160 mg, 0.5 mmol) in acetic acid (4.4 mL, 77.7 mmol) was added water (0.93 mL, 51.8 mmol) at ambient temperature. The reaction mixture was heated to reflux for 16 h. The reaction mixture was concentrated under reduced pressure, dissolved in EtOAc, washed with water, brine, dried over anhydrous Na₂SO₄, filtered, and concentrated under reduced pressure. The product was purified by reverse phase prep-HPLC and lyophilized to afford 2-((1-oxo-4-(o-tolyl)-1,2-dihydroisoquinolin-7-yl)oxy)acetonitrile (Example 36, 70 mg). LCMS (ESI) Calcd. for C₁₈H₁₄N₂O₂: 290, found [M+H]⁺=291. ¹H NMR (400 MHz, DMSO-d₆) δ 11.48 (s, 1H), 7.86 (d, 1H), 7.38-7.35 (m, 3H), 7.32-7.27 (m, 1H), 7.21-7.19 (d, 1H), 6.98-6.95 (m, 2H), 5.32 (s, 2H), 2.04 (s, 3H).

### Example 37: Synthesis of 2-((4-(2-chloro-4-fluorophenyl)-1-oxo-1,2-dihydroisoquinolin-7-yl)oxy)acetonitrile

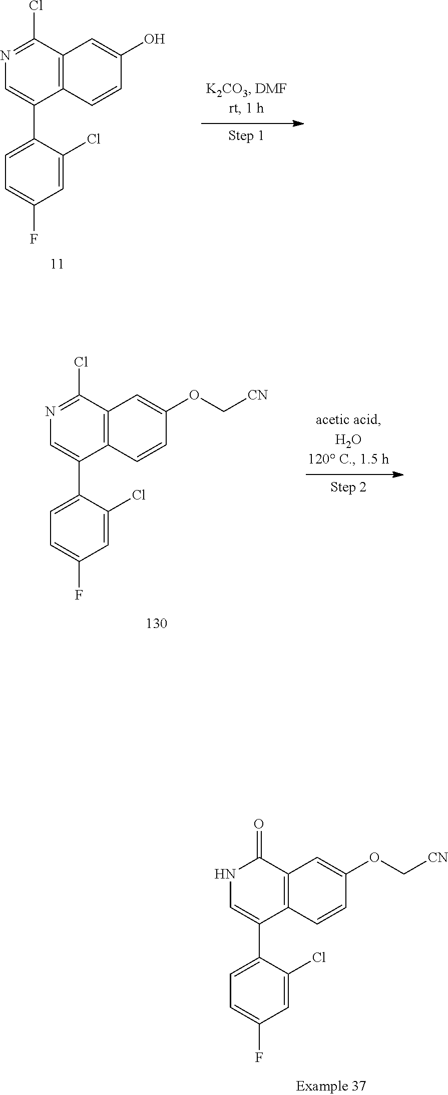 [SMILES: Oc1ccc2c(-c3ccc(F)cc3Cl)cnc(Cl)c2c1 ; N#CCOc1ccc2c(-c3ccc(F)cc3Cl)cnc(Cl)c2c1 ; N#CCOc1ccc2c(-c3ccc(F)cc3Cl)c[nH]c(=O)c2c1]

Synthesis of 2-((1-chloro-4-(2-chloro-4-fluorophenyl)isoquinolin-7-yl)oxy)acetonitrile, 130 [Step 1]: To a stirred solution of 1-chloro-4-(2-chloro-4-fluoro-phenyl)isoquinolin-7-ol (11, 50 mg, 0.2 mmol) in DMF (5 mL) was added K₂CO₃ (90 mg, 0.7 mmol) followed by 2-bromoacetonitrile (0.023 mL, 0.3 mmol). The reaction mixture was stirred at ambient temperature for 2 h. The reaction mixture was partitioned between EtOAc and water. The combined organic layers were washed with water, brine, dried over anhydrous Na₂SO₄, and concentrated under reduced pressure. The product was purified by flash column chromatography to afford 2-((1-chloro-4-(2-chloro-4-fluorophenyl)isoquinolin-7-yl)oxy)acetonitrile (130, 42 mg). LCMS (ESI) Calcd. for C₁₇H₉Cl₂FN₂O: 346, found [M+H]⁺=347. ¹H NMR (400 MHz, DMSO-d₆) δ 7.95 (d, 1H), 7.81 (d, 1H), 7.71 (dd, 1H), 7.64-7.60 (m, 1H), 7.58-7.56 (d, 1H), 7.47-7.41 (m, 2H), 5.47 (d, 2H).

Synthesis of 2-((4-(2-chloro-4-fluorophenyl)-1-oxo-1,2-dihydroisoquinolin-7-yl)oxy)acetonitrile, Example 37 [Step 2]: To a stirred solution of 2-((1-chloro-4-(2-chloro-4-fluorophenyl)isoquinolin-7-yl)oxy)acetonitrile (130, 120 mg, 0.3 mmol) in acetic acid (3.0 mL, 51.8 mmol) was added water (0.6 mL, 34.6 mmol) at ambient temperature. The reaction mixture was heated to reflux for 16 h. The reaction mixture was diluted with water and extracted with EtOAc. The organic layer was washed with brine, dried over anhydrous Na₂SO₄, filtered, and concentrated under reduced pressure. The product was purified by reverse phase prep-HPLC to afford 2-((4-(2-chloro-4-fluorophenyl)-1-oxo-1,2-dihydroisoquinolin-7-yl)oxy)acetonitrile (Example 37, 37 mg). LCMS (ESI) Calcd. for C₁₇H₁₀ClFN₂O₂: 328, found [M+H]⁺=329. ¹H NMR (400 MHz, DMSO-d₆) δ 11.60 (br s, 1H), 7.85 (d, 1H), 7.61 (dd, 1H), 7.51-7.48 (m, 1H), 7.39-7.32 (m, 2H), 7.09 (s, 1H), 7.01 (d, 1H), 5.32 (s, 2H).

### Examples 38-39: Synthesis of 7-(aminomethyl)-4-(o-tolyl)isoquinolin-1(2H)-one and N-((1-oxo-4-(o-tolyl)-1,2-dihydroisoquinolin-7-yl)methyl)acetamide

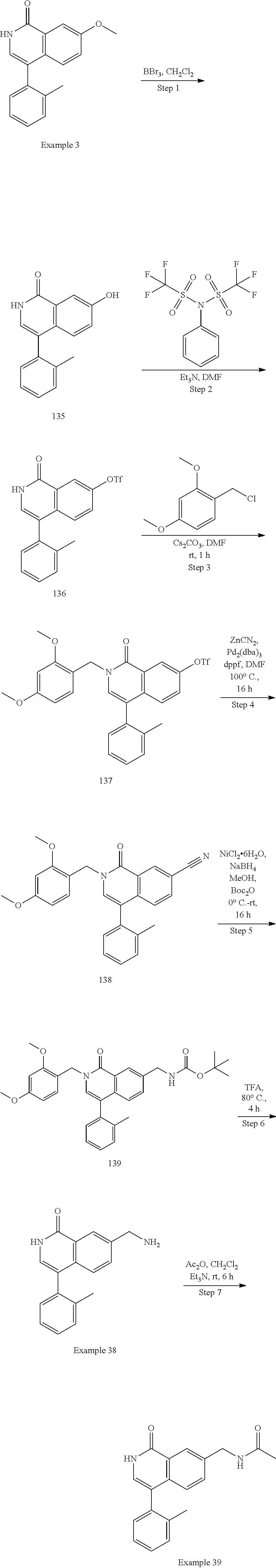

Synthesis of 7-hydroxy-4-(o-tolyl)isoquinolin-1(2H)-one, 135 [Step 1]: To a stirred solution of 7-methoxy-4-(o-tolyl)isoquinolin-1(2H)-one (Example 3, 1.5 g, 5.7 mmol) in CH₂Cl₂ (7 mL) was added dropwise BBr₃ (17 ml, 17.0 mmol, 1M in CH₂Cl₂) at 0° C. The reaction mixture was stirred at ambient temperature for 3 h. The reaction mixture was cooled to 0° C. and quenched with MeOH. The reaction mixture was concentrated under reduced pressure, diluted with CH₂C₂, washed with water, brine, dried over anhydrous Na₂SO₄, filtered, and concentrated under reduced pressure to afford 7-hydroxy-4-(o-tolyl)isoquinolin-1(2H)-one (135, 1.4 g). LCMS (ESI) Calcd. for C₁₆H₁₃NO₂: 251, found [M+H]⁺=252. ¹H NMR (400 MHz, DMSO-d₆) δ 11.20-11.19 (m, 1H), 7.61 (d, 1H), 7.34 (d, 2H), 7.29-7.24 (m, 1H), 7.18 (d, 1H), 7.12-7.09 (dd, 1H), 6.83 (d, 1H), 6.78 (d, 1H), 2.03 (s, 3H).

Synthesis of 1-oxo-4-(o-tolyl)-1,2-dihydroisoquinolin-7-yl trifluoromethanesulfonate, 136 [Step 2]: To a stirred suspension of 7-hydroxy-4-(o-tolyl)isoquinolin-1(2H)-one (135, 500 mg, 2.0 mmol) in DMF (7 mL) was added triethylamine (0.85 mL, 6.0 mmol). The reaction mixture was cooled to 0° C., and N-((difluoromethyl)sulfonyl)-1,1,1-trifluoro-N-phenylmethanesulfonamide (925 mg, 2.4 mmol) was added portion wise. The reaction mixture was warmed to ambient temperature and stirred for 16 h. The reaction mixture was quenched with ice cold water and stirred for 30 min. The reaction mixture was extracted with EtOAc. The combined organic layers were washed with water, brine, dried over anhydrous Na₂SO₄, filtered, and concentrated under reduced pressure. The product was purified by flash chromatography to afford 1-oxo-4-(o-tolyl)-1,2-dihydroisoquinolin-7-yl trifluoromethanesulfonate (136, 550 mg). LCMS (ESI) Calcd. for C₁₇H₁₂F₃NO₄S: 383, found [M+H]⁺=384. ¹H NMR (400 MHz, DMSO-d₆) δ 11.77 (d, 1H), 8.22 (d, 1H), 7.78 (dd, 1H), 7.38 (d, 2H), 7.33-7.28 (m, 1H), 7.24 (d, 1H), 7.17-7.13 (m, 2H), 2.05 (s, 3H).

Synthesis of 2-(2,4-dimethoxybenzyl)-1-oxo-4-(o-tolyl)-1,2-dihydroisoquinolin-7-yl trifluoromethanesulfonate, 137 [Step 3]: To a solution of 1-oxo-4-(o-tolyl)-1,2-dihydroisoquinolin-7-yl trifluoromethanesulfonate (136, 500 mg, 1.30 mmol) in DMF (4 mL) was added Cs₂CO₃ (850 mg, 2.6 mmol) and an ether solution of 1-(chloromethyl)-2,4-dimethoxy-benzene (730 mg, 3.9 mmol) at 0° C. The reaction mixture was warmed to ambient temperature and stirred for 16 h. The reaction mixture was quenched with water and extracted with EtOAc. The combined organic layers were washed with water, brine, dried over anhydrous Na₂SO₄, filtered, and concentrated under reduced pressure. The product was purified by flash chromatography to afford 2-(2,4-dimethoxybenzyl)-1-oxo-4-(o-tolyl)-1,2-dihydroisoquinolin-7-yl trifluoromethanesulfonate (137, 560 mg). LCMS (ESI) Calcd. for C₂₆H₂₂F₃NO₆S: 533, found [M+H]⁺=534. ¹H NMR (400 MHz, DMSO-d₆) δ 8.24 (d, 1H), 7.79 (dd, 1H), 7.46 (s, 1H), 7.39 (d, 2H), 7.32 (br s, 1H), 7.24 (d, 1H), 7.15 (dd, 2H), 6.58 (d, 1H), 6.49 (dd, 1H), 5.08 (br s, 2H), 3.75 (d, 6H), 2.05 (s, 3H).

Synthesis 2-(2,4-dimethoxybenzyl)-1-oxo-4-(o-tolyl)-1,2-dihydroisoquinoline-7-carbonitrile, 138 [Step 4]: A solution of 2-(2,4-dimethoxybenzyl)-1-oxo-4-(o-tolyl)-1,2-dihydroisoquinolin-7-yl trifluoromethanesulfonate (137, 600 mg, 1.1 mmol) in DMF (7 mL) was degassed with argon for 10 min. To the solution was added zinc cyanide (200 mg, 1.7 mmol), Pd₂(dba)₃ (100 mg, 0.1 mmol), and dppf (125 mg, 0.2 mmol). The reaction mixture was heated at 100° C. for 16 h. The reaction mixture was cooled to ambient temperature and filtered through celite. The filtrate was diluted with EtOAc, washed with water, brine, dried over anhydrous Na₂SO₄, filtered, and concentrated under reduced pressure. The product was purified by flash chromatography to afford 2-(2,4-dimethoxybenzyl)-1-oxo-4-(o-tolyl)-1,2-dihydroisoquinoline-7-carbonitrile (138, 300 mg). LCMS (ESI) Calcd. for C₂₆H₂₂N₂O₃: 410, found [M+H]⁺=411. ¹H NMR (400 MHz, DMSO-d₆) δ 8.63 (d, 1H), 8.00-7.97 (dd, 1H), 7.54 (s, 1H), 7.39 (d, 2H), 7.34-7.30 (m, 1H), 7.22 (d, 1H), 7.12 (t, 2H), 6.58 (d, 1H), 6.50-6.47 (dd, 1H), 5.13-5.04 (m, 2H), 3.77 (s, 3H), 3.73 (s, 3H), 2.03 (s, 3H).

Synthesis tert-butyl ((2-(2,4-dimethoxybenzyl)-1-oxo-4-(o-tolyl)-1,2-dihydroisoquinolin-7-yl)methyl)carbamate, 139 [Step 5]: To a stirred solution of 2-(2,4-dimethoxybenzyl)-1-oxo-4-(o-tolyl)-1,2-dihydroisoquinoline-7-carbonitrile (138, 350 mg, 0.9 mmol) in MeOH (3 mL) was added NiCl₂·6H₂O (11 mg, 0.1 mmol) and Boc₂O (0.4 mL, 1.7 mmol). NaBH₄ (225 mg, 6.0 mmol) was added portion wise to the reaction mixture under argon atmosphere. The reaction mixture was stirred at ambient temperature for 16 h. The reaction mixture was concentrated under reduced pressure. The reaction mixture was extracted in EtOAc, washed with water, brine, dried over anhydrous Na₂SO₄, filtered, and concentrated under reduced pressure. The product was purified by flash chromatography to afford tert-butyl ((2-(2,4-dimethoxybenzyl)-1-oxo-4-(o-tolyl)-1,2-dihydroisoquinolin-7-yl)methyl)carbamate (139, 300 mg). LCMS (ESI) Calcd. for C₃₁H₃₄N₂O₅: 514, found [M+H]⁺=515. ¹H NMR (400 MHz, DMSO-d₆) δ 8.18 (s, 1H), 7.51 (d, 2H), 7.36 (d, 2H), 7.29 (m, 1H), 7.24 (s, 1H), 7.19 (d, 1H), 7.04 (d, 1H), 6.96 (d, 1H), 6.58 (d, 1H), 6.48 (dd, 1H), 5.12-5.02 (m, 2H), 4.24 (d, 2H), 3.77 (s, 3H), 3.72 (s, 3H), 2.03 (s, 3H), 1.38 (s, 9H).

Synthesis of 7-(aminomethyl)-4-(o-tolyl)isoquinolin-1(2H)-one, Example 38 [Step 6]: A stirred mixture of tert-butyl ((2-(2,4-dimethoxybenzyl)-1-oxo-4-(o-tolyl)-1,2-dihydroisoquinolin-7-yl)methyl)carbamate (139, 50 mg, 0.1 mmol) and TFA (1.1 ml, 14.6 mmol) was heated at 80° C. for 4 h. The reaction mixture was concentrated under reduced pressure. The product was neutralized with a NaHCO₃ solution and extracted with EtOAc. The combined organic layers were washed with brine, dried over anhydrous Na₂SO₄, filtered, and concentrated under reduced pressure. The product was purified by reverse phase prep-HPLC and lyophilized to afford 7-(aminomethyl)-4-(o-tolyl)isoquinolin-1(2H)-one (Example 38, 15 mg). LCMS (ESI) Calcd. for C₁₇H₁₆N₂O: 264, found [M+H]⁺=265. ¹H NMR (400 MHz, DMSO-d₆) δ 11.30 (s, 1H), 8.25 (s, 1H), 7.60 (dd, 1H), 7.35 (d, 2H), 7.29-7.27 (m, 1H), 7.20 (d, 1H), 6.95-6.90 (m, 2H), 3.83 (s, 2H), 2.04 (s, 3H).

Synthesis of N-((1-oxo-4-(o-tolyl)-1,2-dihydroisoquinolin-7-yl)methyl)acetamide, Example 39 [Step 7]: To a stirred solution of 7-(aminomethyl)-4-(o-tolyl)isoquinolin-1(2H)-one (Example 38, 75 mg, 0.3 mmol) in CH₂Cl₂ (2 mL) was added triethylamine (0.12 mL, 0.9 mmol) and acetic anhydride (0.04 mL, 0.4 mmol). The reaction mixture was stirred at ambient temperature for 4 h. The reaction mixture was concentrated under reduced pressure. The reaction mixture was extracted in EtOAc, washed with water, brine, dried over anhydrous Na₂SO₄, filtered, and concentrated under reduced pressure. The product was purified by reverse phase prep-HPLC and lyophilized to afford N-((1-oxo-4-(o-tolyl)-1,2-dihydroisoquinolin-7-yl)methyl)acetamide (Example 39, 35 mg). LCMS (ESI) Calcd. for C₁₉H₁₈N₂O₂: 306, found [M+H]⁺=307. ¹H NMR (400 MHz, DMSO-d₆) δ 11.64-11.16 (m, 1H), 8.45 (t, 1H), 8.16 (s, 1H), 7.51 (dd, 1H), 7.35 (d, 2H), 7.30-7.26 (m, 1H), 7.20 (d, 1H), 6.98 (s, 1H), 6.94 (d, 1H), 4.36 (d, 2H), 2.03 (s, 3H), 1.88 (s, 3H).

### Examples 40-41: Synthesis of ethyl 2-methyl-3-(1-oxo-4-(o-tolyl)-1,2-dihydroisoquinolin-7-yl)propanoate and 7-(2-methyl-3-oxo-3-(piperidin-1-yl)propyl)-4-(o-tolyl)isoquinolin-1(2H)-one

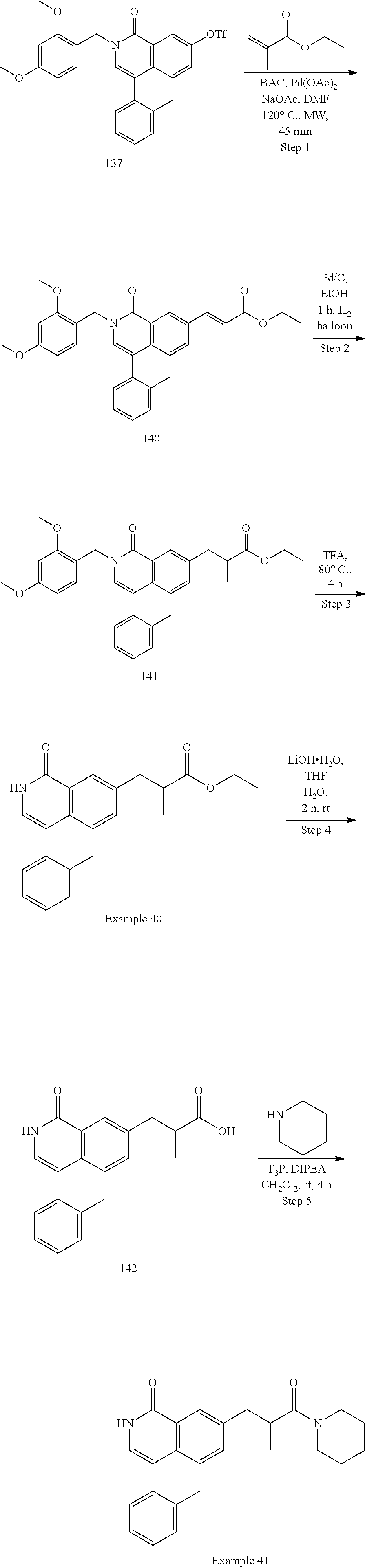

Synthesis of ethyl (E)-3-(2-(2,4-dimethoxybenzyl-1-oxo-4-(o-tolyl)-1,2-dihydroisoquinolin-7-yl)-2-methylacrylate, 140 [Step 1]: To a stirred solution of 2-(2,4-dimethoxybenzyl)-1-oxo-4-(o-tolyl)-1,2-dihydroisoquinolin-7-yl trifluoromethanesulfonate (137, 100 mg, 0.2 mmol) in DMF (5 mL) was added NaOAc (31 mg, 0.4 mmol) and TBAC (104 mg, 0.4 mmol). The reaction mixture was purged with argon for 5 min., and ethyl methacrylate (0.05 mL, 0.4 mmol) was added followed by Pd(OAc)₂ (4.2 mg, 0.02 mmol). The reaction mixture was irradiated in microwave at 120° C. for 45 min. The reaction mixture was filtered through celite and concentrated under reduced pressure. and the product was partitioned between EtOAc and water. The organic part was washed with brine, dried over anhydrous Na₂SO₄, filtered, and concentrated under reduced pressure. The product was purified by flash chromatography to afford ethyl (E)-3-(2-(2,4-dimethoxybenzyl-1-oxo-4-(o-tolyl)-1,2-dihydroisoquinolin-7-yl)-2-methylacrylate (140, 64 mg). LCMS (ESI) Calcd. for C₃₁H₃₁NO₅: 497, found [M+H]⁺=498.

Synthesis of ethyl 3-(2-(2,4-dimethoxybenzyl)-1-oxo-4-(o-tolyl)-1,2-dihydroisoquinolin-7-yl)-2-methylpropanoate, 141 [Step 2]: To a degassed solution of ethyl (E)-3-(2-(2,4-dimethoxybenzyl-1-oxo-4-(o-tolyl)-1,2-dihydroisoquinolin-7-yl)-2-methylacrylate (140, 160 mg, 0.3 mmol) in ethanol (5 mL) was added 10% Pd/C (34 mg, 0.3 mmol). After stirring for 1 h, the reaction mixture was filtered through celite, diluted in ethanol, and concentrated to afford ethyl 3-(2-(2,4-dimethoxybenzyl)-1-oxo-4-(o-tolyl)-1,2-dihydroisoquinolin-7-yl)-2-methylpropanoate (141, 150 mg). LCMS (ESI) Calcd. for C₃₁H₃₃NO₅: 499, found [M+H]⁺=501.

Synthesis of ethyl 2-methyl-3-(1-oxo-4-(o-tolyl)-1,2-dihydroisoquinolin-7-yl)propanoate, Example 40 [Step 3]: A solution of 3-(2-(2,4-dimethoxybenzyl)-1-oxo-4-(o-tolyl)-1,2-dihydroisoquinolin-7-yl)-2-methylpropanoate (141, 200 mg, 0.4 mmol) in TFA (2.5 mL, 32.5 mmol) was stirred at 85° C. for 4 h. The reaction mixture was cooled to ambient temperature and concentrated under reduced pressure. The reaction mixture was washed with MeOH and dried under high vacuum. The reaction mixture was partitioned between EtOAc and water. The organic phase was washed with brine, dried over anhydrous Na₂SO₄, filtered, and concentrated under reduced pressure. The product was purified by reverse phase prep-HPLC to afford ethyl 2-methyl-3-(1-oxo-4-(o-tolyl)-1,2-dihydroisoquinolin-7-yl)propanoate (Example 40, 117 mg). LCMS (ESI) Calcd. for C₂₂H₂₃NO₃: 349, found [M+H]⁺=350. ¹H NMR (400 MHz, DMSO-d₆) δ 11.33 (d, 1H), 8.07 (s, 1H), 7.47 (t, 1H), 7.35 (d, 2H), 7.30-7.26 (m, 1H), 7.20 (d, 1H), 6.96 (d, 1H), 6.90 (d, 1H), 4.01 (dd, 2H), 3.00-2.94 (m, 1H), 2.86-2.81 (m, 1H), 2.79-2.75 (m, 1H), 2.03 (s, 3H), 1.10-1.04 (m, 6H).

Synthesis of 2-methyl-3-(1-oxo-4-(o-tolyl)-1,2-dihydroisoquinolin-7-yl-propanoic acid, 142 [Step 4]: To a stirred solution of ethyl 2-methyl-3-(1-oxo-4-(o-tolyl)-1,2-dihydroisoquinolin-7-yl)propanoate (Example 40, 120 mg, 0.3 mmol) in THF (2 mL) and water (0.8 mL) mixture was added LiOH·H₂O (33 mg, 1.4 mmol) at ambient temperature and stirred for 2 h. The product was diluted with water, acidified with 10% aq. citric acid solution, and extracted with EtOAc. The combined organic layer was washed with brine, dried over anhydrous Na₂SO₄, and concentrated under reduced pressure to afford 2-methyl-3-(1-oxo-4-(o-tolyl)-1,2-dihydroisoquinolin-7-yl-propanoic acid (142, 100 mg). LCMS (ESI) Calcd. for C₁₉H₁₇NO₄: 323, found [M+H]⁺=322.

Synthesis of 7-(2-methyl-3-oxo-3-(piperidin-1-yl)propyl)-4-(o-tolyl)isoquinolin-1(2H)-one, Example 41 [Step 5]: To a stirred solution of 2-methyl-3-(1-oxo-4-(o-tolyl)-1,2-dihydroisoquinolin-7-yl-propanoic acid (142, 60 mg, 0.2 mmol) in CH₂Cl₂ (5 mL) was added DIPEA (0.2 mL, 1.2 mmol) followed by piperidine (0.1 mL, 1.2 mmol). T₃P (0.2 mL, 0.6 mmol, 50% in EtOAc) was added at 0° C. The reaction mixture was stirred at ambient temperature for 4 h. The reaction mixture was concentrated under reduced pressure. The product was partitioned between EtOAc and water. The organic phase was washed with brine, dried over anhydrous Na₂SO₄, filtered, and concentrated under reduced pressure. The product was purified by reverse phase prep-HPLC to afford 7-(2-methyl-3-oxo-3-(piperidin-1-yl)propyl)-4-(o-tolyl)isoquinolin-1(2H)-one (Example 41, 36 mg). LCMS (ESI) Calcd. for C₂₅H₂₈N₂O₂: 388, found [M+H]⁺=389. ¹H NMR (400 MHz, DMSO-d₆) δ 11.30 (d, 1H), 8.08 (d, 1H), 7.50-7.43 (dd, 1H), 7.35 (d, 2H), 7.30-7.27 (m, 1H), 7.19 (d, 1H), 6.94 (d, 1H), 6.87-6.85 (dd, 1H), 3.31 (s, 1H), 3.29 (d, 1H), 3.17-3.11 (m, 3H), 2.97-2.92 (m, 1H), 2.72-2.67 (m, 1H), 2.02 (s, 3H), 1.43-1.14 (m, 5H), 1.03 (d, 3H), 0.90 (br s, 1H).

### Example 42: Synthesis of 7-((1-acetylpiperidin-3-yl)oxy)-4-(o-tolyl)isoquinolin-1(2H)-one

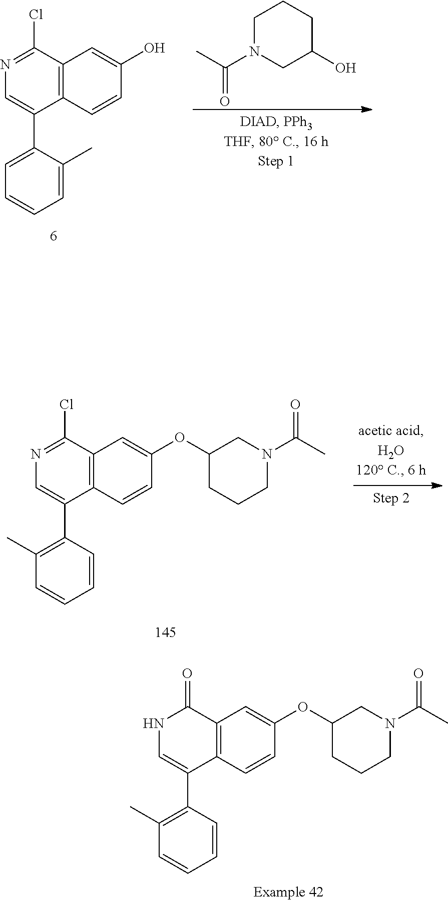

Synthesis of 1-(3-((1-chloro-4-(o-tolyl)isoquinolin-7-yl)oxy)piperidin-1-yl)ethan-1-one, 145 [Step 1]: To a stirred solution of 1-chloro-4-(o-tolyl)isoquinolin-7-ol (6, 200 mg, 0.7 mmol) and 1-(3-hydroxypiperidin-1-yl)ethan-1-one (160 mg, 1.1 mmol) in THF (6 mL) was added PPh₃ (585 mg, 2.2 mmol) followed by DIAD (0.5 mL, 2.2 mmol). The reaction mixture was heated at 90° C. for 16 h. The reaction mixture was concentrated under reduced pressure and partitioned between EtOAc and water. The organic phase was washed with brine, dried over anhydrous Na₂SO₄, filtered, and concentrated under reduced pressure. The product was purified by reverse phase prep-HPLC to afford 1-(3-((1-chloro-4-(o-tolyl)isoquinolin-7-yl)oxy)piperidin-1-yl)ethan-1-one (145, 80 mg). LCMS (ESI) Calcd. for C₂₃H₂₃ClN₂O₂: 394, found [M+H]⁺=395. ¹H NMR (400 MHz, DMSO-d₆) δ 8.04 (s, 1H), 7.69 (s, 1H), 7.51 (s, 1H), 7.47 (d, 2H), 7.35-7.33 (m, 2H), 7.25 (d, 1H), 5.75 (d, 1H), 4.85 (s, 1H), 4.62 (s, 1H), 4.01 (s, 1H), 3.79 (d, 1H), 3.56-3.43 (m, 2H), 3.42-3.29 (m, 1H), 2.08 (s, 2H), 2.03-1.82 (m, 4H), 1.69-1.61 (m, 1H), 1.58-1.48 (m, 2H), 1.22-1.14 (m, 1H).

Synthesis of 7-((1-acetylpiperidin-3-yl)oxy)-4-(o-tolyl)isoquinolin-1(2H)-one, Example 42 [Step 2]: To a stirred solution of 1-(3-((1-chloro-4-(o-tolyl)isoquinolin-7-yl)oxy)piperidin-1-yl)ethan-1-one (145, 65 mg, 0.2 mmol) in acetic acid (1.4 mL, 25 mmol) was added water (0.3 mL, 16.5 mmol) at ambient temperature. The reaction mixture was heated to reflux for 16 h. The reaction mixture was concentrated under reduced pressure and partitioned between EtOAc and water. The organic phase was washed with brine, dried over anhydrous Na₂SO₄, filtered, and concentrated under reduced pressure. The product was purified by reverse phase prep-HPLC and lyophilized to afford 7-((1-acetylpiperidin-3-yl)oxy)-4-(o-tolyl)isoquinolin-1(2H)-one (Example 42, 30 mg). LCMS (ESI) Calcd. for C₂₃H₂₄N₂O₃: 376, found [M+H]⁺=377. ¹H NMR (400 MHz, DMSO-d₆) 100° C. δ 10.97 (br s, 1H), 7.78 (s, 1H), 7.34 (s, 2H), 7.27-7.19 (m, 3H), 6.95 (d, 1H), 6.84 (s, 1H), 6.54 (br s, 1H), 3.80 (br s, 1H), 3.46 (br s, 3H), 2.07-1.97 (m, 7H), 1.79 (br s, 2H), 1.54 (br s, 1H).

### Example 43: Synthesis of 1-methyl-N-(1-oxo-4-(o-tolyl)-1,2-dihydroisoquinolin-7-yl) piperidine-4-carboxamide

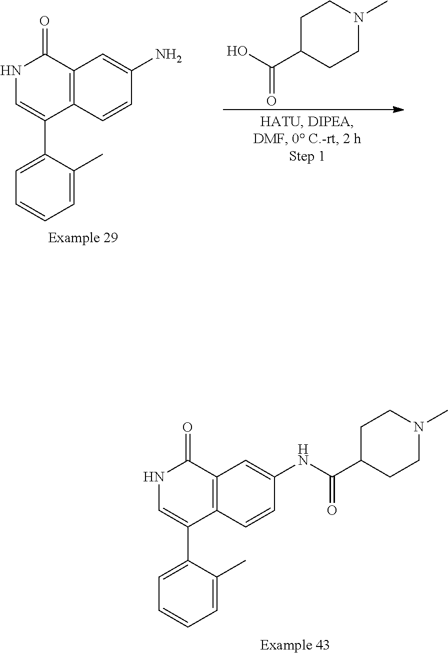

Synthesis of 1-methyl-N-(1-oxo-4-(o-tolyl)-1,2-dihydroisoquinolin-7-yl) piperidine-4-carboxamide, Example 43 [Step 1]: To a solution of 1-methylpiperidine-4-carboxylic acid (70 mg, 0.5 mmol) in DMF (4 ml), was added DIPEA (0.1 mL, 0.8 mmol) and HATU (151 mg, 0.4 mmol). The reaction mixture was stirred for 15 min. at ambient temperature. 7-amino-4-(o-tolyl)-2H-isoquinolin-1-one (Example 29, 100 mg, 0.4 mmol) was added, and the reaction mixture was stirred for 2 h. at ambient temperature. The reaction mixture was diluted with cold water and extracted with EtOAc. The combined organic phase was washed with cold water, brine, dried over anhydrous Na₂SO₄, filtered, and concentrated under reduced pressure to afford the product. The product was purified by reverse phase prep-HPLC and lyophilized to afford 1-methyl-N-(1-oxo-4-(o-tolyl)-1,2-dihydroisoquinolin-7-yl)piperidine-4-carboxamide (Example 43, 30 mg). LCMS (ESI) Calcd. for C₂₃H₂₅N₃O₂: 375, found [M+H]⁺=376. ¹H NMR (400 MHz, DMSO-d₆) δ 11.27 (d, 1H), 10.14 (s, 1H), 8.58 (d, 1H), 7.80 (t, 1H), 7.34 (d, 2H), 7.30-7.26 (m, 1H), 7.19 (d, 1H), 6.89 (t, 2H), 2.81 (d, 2H), 2.30 (t, 1H), 2.15 (s, 3H), 2.03 (s, 3H), 1.90-1.83 (m, 2H), 1.75 (d, 2H), 1.69-1.61 (m, 2H).

### Example 44: Synthesis of 7-(methoxymethyl)-4-(o-tolyl)isoquinolin-1(2H)-one

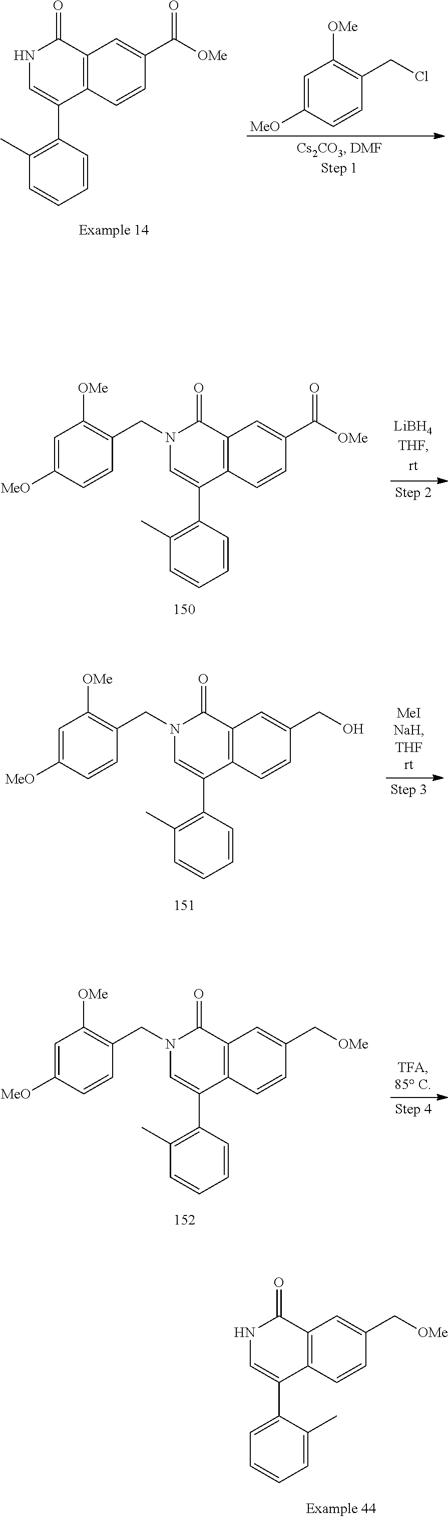 [SMILES: COC(=O)c1ccc2c(-c3ccccc3C)c[nH]c(=O)c2c1 ; COc1ccc(CCl)c(OC)c1 ; COC(=O)c1ccc2c(-c3ccccc3C)cn(Cc3ccc(OC)cc3OC)c(=O)c2c1 ; COc1ccc(Cn2cc(-c3ccccc3C)c3ccc(CO)cc3c2=O)c(OC)c1 ; COCc1ccc2c(-c3ccccc3C)cn(Cc3ccc(OC)cc3OC)c(=O)c2c1 ; COCc1ccc2c(-c3ccccc3C)c[nH]c(=O)c2c1]

Synthesis of methyl 2-(2,4-dimethoxybenzyl)-1-oxo-4-(o-tolyl)-1,2-dihydroisoquinoline-7-carboxylate, 150 [Step 1]: To an oven-dried round bottom flask, charged with methyl 1-oxo-4-(o-tolyl)-1,2-dihydroisoquinoline-7-carboxylate (Example 14, 700 mg, 2.4 mmol) and cesium carbonate (1.6 g, 4.8 mmol), was added DMF (10 mL). The reaction mixture was stirred for 10 min. at 25° C. 1-(chloromethyl)-2,4-dimethoxybenzene (670 mg, 3.6 mmol) was added, and the reaction mixture was stirred at 25° C. for 16 h. The reaction mixture was filtered and partitioned between EtOAc and water. The organic phase was collected, washed with cold brine (×3), dried over anhydrous Na₂SO₄, filtered, and concentrated under reduced pressure. The product was purified by silica gel column chromatography to afford methyl 2-(2,4-dimethoxybenzyl)-1-oxo-4-(o-tolyl)-1,2-dihydroisoquinoline-7-carboxylate (150, 700 mg). LCMS (ESI) Calcd. for C₂₇H₂₅NO₅: 443, found [M+H]⁺=444.

Synthesis of 2-(2,4-dimethoxybenzyl)-7-(hydroxymethyl)-4-(o-tolyl)isoquinolin-1(2H)-one, 151 [Step 2]: To a solution of methyl 2-(2,4-dimethoxybenzyl)-1-oxo-4-(o-tolyl)-1,2-dihydroisoquinoline-7-carboxylate (150, 550 mg, 1.2 mmol) in THF (2 mL) was added LiBH₄ (1.2 mL, 2.5 mmol, 2M solution in THF) at 0° C. The reaction mixture was stirred at 25° C. for 1 h. The reaction mixture was quenched with 2M aq. HCl and extracted with EtOAc (×2). The combined organic layers were dried over anhydrous Na₂SO₄, filtered, and concentrated under reduced pressure. The product was purified by column chromatography over silica gel to afford 2-(2,4-dimethoxybenzyl)-7-(hydroxymethyl)-4-(o-tolyl)isoquinolin-1(2H)-one (151, 320 mg). LCMS (ESI) Calcd. for C₂₆H₂₅NO₄: 415, found [M+H]⁺=416.

Synthesis of 2-(2,4-dimethoxybenzyl)-7-(methoxymethyl)-4-(o-tolyl)isoquinolin-1(2H)-one, 152 [Step 3]: To an oven-dried round bottom flask, charged with 2-(2,4-dimethoxybenzyl)-7-(hydroxymethyl)-4-(o-tolyl)isoquinolin-1(2H)-one (151, 200 mg, 0.5 mmol), was added THF (2 mL), NaH (40 mg, 1.0 mmol, 60% dispersion in mineral oil), and iodomethane (60 μL, 1.0 mmol). The reaction mixture was stirred at 25° C. for 16 h. The reaction mixture was quenched with cold water and extracted using EtOAc (×2). The combined organic layers were dried over anhydrous Na₂SO₄, filtered, and concentrated under reduced pressure. The product was purified by column chromatography over silica gel to afford 2-(2,4-dimethoxybenzyl)-7-(methoxymethyl)-4-(o-tolyl)isoquinolin-1(2H)-one (152, 150 mg). LCMS (ESI) Calcd. for C₂₇H₂₇NO₄: 429, found [M+H]⁺=430.

Synthesis of 7-(methoxymethyl)-4-(o-tolyl)isoquinolin-1(2H)-one, Example 44 [Step 4]: A solution of 2-(2,4-dimethoxybenzyl)-7-(methoxymethyl)-4-(o-tolyl)isoquinolin-1(2H)-one (152, 150 mg, 0.3 mmol) in trifluoroacetic acid (2 mL) was stirred at 85° C. for 1.5 h. The reaction mixture was cooled to ambient temperature and concentrated under reduced pressure. The product was purified using reverse phase prep-HPLC and lyophilized to afford 7-(methoxymethyl)-4-(o-tolyl)isoquinolin-1(2H)-one (Example 44, 20.3 mg). LCMS (ESI) Calcd. for C₁₈H₁₇NO₂: 279, found [M+H]⁺=280. ¹H NMR (400 MHz, DMSO-d₆): δ 11.40 (s, 1H), 8.20 (s, 1H), 7.58 (d, 1H), 7.36-7.35 (m, 2H), 7.29-7.28 (m, 1H), 7.22-7.20 (m, 1H), 7.00-6.95 (m, 2H), 4.54 (s, 2H), 3.53 (s, 3H) 2.04 (s, 3H).

### Example 45: Synthesis of N-methyl-N-((1-oxo-4-(o-tolyl)-1,2-dihydroisoquinolin-7-yl)methyl)acetamide

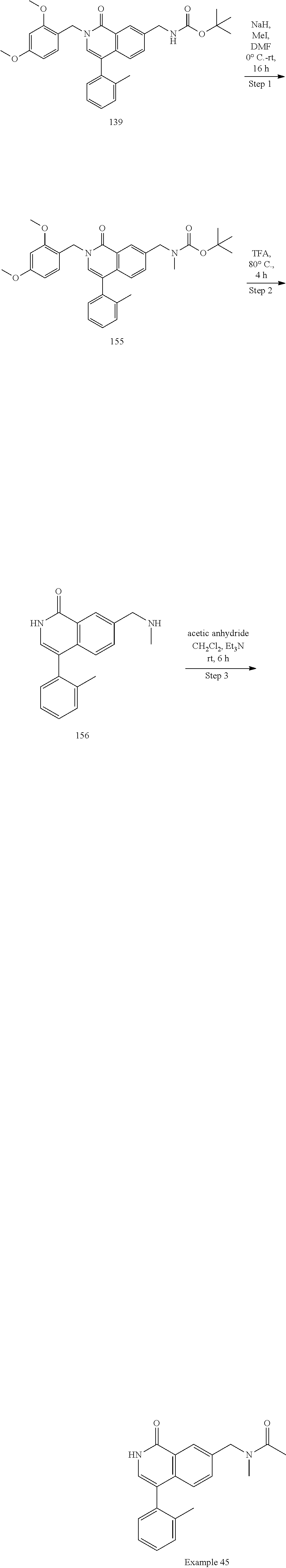

Synthesis of tert-butyl ((2-(2,4-dimethoxybenzyl)-1-oxo-4-(o-tolyl)-1,2-dihydroisoquinolin-7-yl)methyl)carbamate, 155 [Step 1]: To a stirred solution of tert-butyl ((2-(2,4-dimethoxybenzyl)-1-oxo-4-(o-tolyl)-1,2-dihydroisoquinolin-7-yl)methyl)carbamate (139, 100 mg, 0.2 mmol) in DMF (2 mL) was added NaH (16 mg, 0.4 mmol, 60% dispersion in mineral oil) at 0° C. The reaction mixture was stirred at 0° C. for 10 min., and Mel (0.06 mL, 1 mmol) was added. The reaction mixture was stirred at ambient temperature for 16 h. The reaction mixture was quenched with water and extracted with EtOAc. The combined organic layers were dried over anhydrous Na₂SO₄, filtered, and concentrated under reduced pressure. The product was purified by flash chromatography to afford tert-butyl ((2-(2,4-dimethoxybenzyl)-1-oxo-4-(o-tolyl)-1,2-dihydroisoquinolin-7-yl)methyl)carbamate (155, 70 mg). LCMS (ESI) Calcd. for C₃₂H₃₆N₂O₅: 528, found [M+H]⁺=529. ¹H NMR (400 MHz, DMSO-d₆) δ 8.19-8.13 (m, 1H), 7.49 (s, 1H), 7.36 (m, 2H), 7.31 (m, 2H), 7.20 (d, 1H), 7.06 (d, 1H), 7.00 (d, 1H), 6.58 (d, 1H), 6.48 (dd, 1H), 5.11-5.02 (m, 2H), 4.49 (s, 2H), 3.78 (s, 3H), 3.72 (s, 3H), 2.77 (s, 3H), 2.04 (s, 3H), 1.41 (d, 9H).

Synthesis of 7-((methylamino)methyl)-4-(o-tolyl)isoquinolin-1(2H)-one, 156 [Step 2]: A stirred mixture of tert-butyl ((2-(2,4-dimethoxybenzyl)-1-oxo-4-(o-tolyl)-1,2-dihydroisoquinolin-7-yl)methyl)carbamate (155, 120 mg, 0.2 mmol) and TFA (2.0 mL, 26.1 mmol) was heated at 80° C. for 4 h. The reaction mixture was concentrated under reduced pressure. The reaction mixture was neutralized with an aq. NaHCO₃ solution and extracted with EtOAc. The organic phase was washed with water, brine, dried over anhydrous Na₂SO₄, filtered, and concentrated under reduced pressure to afford 7-(methylaminomethyl)-4-(o-tolyl)isoquinolin-1(2H)-one (156, 60 mg). LCMS (ESI) Calcd. for C₁₈H₁₈N₂O: 278, found [M+H]⁺=279.

Synthesis of N-methyl-N-((1-oxo-4-(o-tolyl)-1,2-dihydroisoquinolin-7-yl)methyl)acetamide, Example 45 [Step 3]: To a stirred solution of 7-(methylaminomethyl)-4-(o-tolyl)-2H-isoquinolin-1-one (156, 75 mg, 0.3 mmol) in CH₂Cl₂ (2 mL) was added triethylamine (0.11 mL, 0.8 mmol) and acetic anhydride (0.04 mL, 0.4 mmol) at ambient temperature and stirred for 4 h. The reaction mixture was diluted with EtOAc and washed with water, brine, dried over anhydrous Na₂SO₄, and evaporated under vacuum. The product was purified by reverse phase prep-HPLC and lyophilized to afford N-methyl-N-((1-oxo-4-(o-tolyl)-1,2-dihydroisoquinolin-7-yl)methyl)acetamide (Example 45, 30 mg). LCMS (ESI) Calcd. for C₂₀H₂₀N₂O₂: 320, found [M+H]⁺=321. ¹H NMR (400 MHz, DMSO-d₆) δ 11.43-11.38 (m, 1H), 8.11-8.07 (d, 1H), 7.51-7.49 (m, 1H), 7.35 (br s, 2H), 7.28 (m, 1H), 7.20 (m, 1H), 7.01-6.98 (d, 1H), 6.95 (d, 1H), 4.69-4.60 (d, 2H), 2.93-2.81 (m, 3H), 2.07 (s, 6H).

### Example 46: Synthesis of 7-(1-morpholino-1-oxopropan-2-yl)amino)-4-(o-tolyl)isoquinolin-1(2H)-one

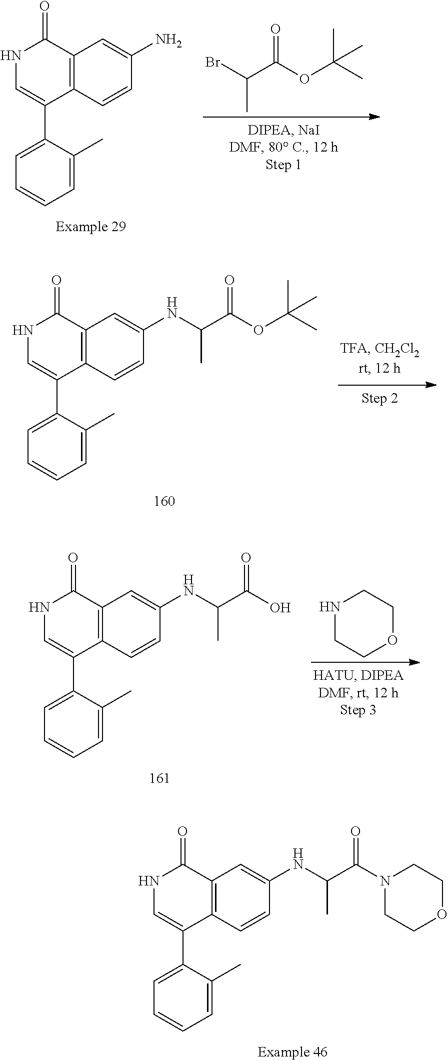

Synthesis of 7-amino-4-(o-tolyl)-1,2-dihydroisoquinolin-7-yl)alaninate, 160 [Step 1]: To a solution of 7-amino-4-(o-tolyl)isoquinolin-1(2H) (Example 29, 1.0 g, 4.0 mmol) in DMF (10 mL) was added DIPEA (1.6 ml, 12 mmol), tert-butyl-2-bromopropanoate (2.6 g, 12 mmol), and NaI (130 mg, 0.8 mmol). The reaction mixture was stirred at 80° C. for 12 h. The reaction mixture was partitioned between EtOAc and water. The organic layer was washed with water (×2) and brine, dried over anhydrous Na₂SO₄, filtered, and concentrated under reduced pressure. The product was purified by column chromatography to afford 7-amino-4-(o-tolyl)-1,2-dihydroisoquinolin-7-yl)alaninate (160, 900 mg). LCMS (ESI) Calcd. for C₂₃H₂₆N₂O₃: 378, found [M+H]⁺=379. ¹H NMR (400 MHz, DMSO-d₆) δ 11.00 (d, 1H), 7.31 (d, 2H), 7.26 (d, 2H), 7.16 (d, 1H), 6.99 (d, 1H), 6.73 (d, 1H), 6.67 (d, 1H), 6.42 (d, 1H), 4.02 (d, 1H), 2.03 (d, 3H), 1.38 (d, 12H).

Synthesis of (1-oxo-4-(o-tolyl)-1,2-dihydroisoquinolin-7-yl)alanine, 161 [Step 2]: To a stirred solution of 7-amino-4-(o-tolyl)-1,2-dihydroisoquinolin-7-yl)alaninate (160, 250 mg, 0.7 mmol) in CH₂Cl₂ (3 mL) was added TFA (1.0 mL, 13.2 mmol). The reaction mixture was stirred at ambient temperature for 2 h. The reaction mixture was concentrated under reduced pressure. Toluene (5 mL) was added and coevaporated to afford (1-oxo-4-(o-tolyl)-1,2-dihydroisoquinolin-7-yl)alanine (161, 150 mg). LCMS (ESI) Calcd. for C₁₉H₁₈N₂O₃: 322, found [M+H]⁺=323. ¹H NMR (400 MHz, DMSO-d₆) δ 11.00 (d, 1H), 7.31 (d, 2H), 7.27-7.24 (m, 2H), 7.22 (d, 1H), 7.16 (d, 1H), 7.01 (d, 1H), 6.73 (d, 1H), 6.65 (d, 1H), 2.03 (d, 3H), 1.40 (d, 3H).

Synthesis of 7-(1-morpholino-1-oxopropan-2-yl)amino)-4-(o-tolyl)isoquinolin-1(2H)-one, Example 46 [Step 3]: To a solution of (1-oxo-4-(o-tolyl)-1,2-dihydroisoquinolin-7-yl)alanine (161, 150 mg, 0.5 mmol) and morpholine (0.05 mL, 0.055 mmol) in DMF (4 mL), was added DIPEA (150 mg, 1.16 mmol) and HATU (177 mg, 0.46 mmol). The reaction mixture was stirred for 2 h. at ambient temperature. The reaction mixture was diluted with cold water and extracted with EtOAc. The combined organic layers were washed with cold water, brine, dried over anhydrous Na₂SO₄, filtered, and concentrated under reduced pressure. The product was purified by reverse phase prep-HPLC and lyophilized to afford 7-(1-morpholino-1-oxopropan-2-yl)amino)-4-(o-tolyl)isoquinolin-1(2H)-one. (Example 46, 85 mg). LCMS (ESI) Calcd. for C₂₃H₂₅N₃O₃: 391, found [M+H]⁺=392. ¹H NMR (400 MHz, DMSO-d₆) δ 11.02 (d, 1H), 7.32-7.24 (m, 4H), 7.16 (t, 1H), 7.09 (t, 1H), 6.71 (d, 1H), 6.67 (t, 1H), 6.26 (d, 1H), 4.58 (t, 1H), 3.74 (s, 2H), 3.58 (d, 5H), 2.04 (s, 3H), 1.27 (d, 3H).

### Example 47: Synthesis of N,N-2-trimethyl-3-(1-oxo-4-(o-tolyl)-1,2-dihydroisoquinolin-7-yl)propenamide

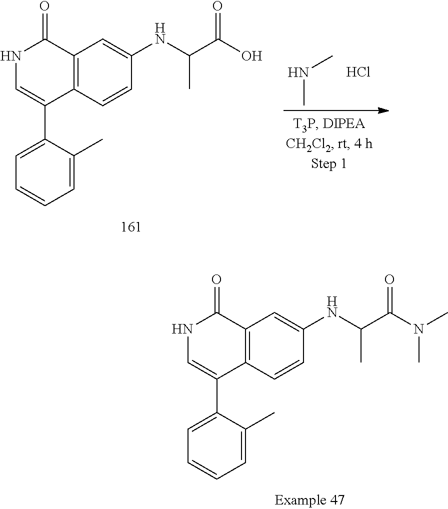

Synthesis of N,N-2-trimethyl-3-(1-oxo-4-(o-tolyl)-1,2-dihydroisoquinolin-7-yl)propenamide, Example 47 [Step 1]: To a stirred solution of (1-oxo-4-(o-tolyl)-1,2-dihydroisoquinolin-7-yl)alanine (161, 170 mg, 0.5 mmol) in CH₂Cl₂ (5 mL) was added DIPEA (0.5 mL, 3 mmol), methylamine.HCl (344 mg, 4.2 mmol), and T₃P (0.5 mL, 0.8 mmol, 50% in EtOAc) at 0° C. The reaction mixture was stirred at ambient temperature for 4 h. The reaction mixture was concentrated under reduced pressure and partitioned between EtOAc and water. The organic phase was washed with brine, dried over anhydrous Na₂SO₄, filtered, and concentrated under reduced pressure. The product was purified by reverse phase prep-HPLC to afford N,N-2-trimethyl-3-(1-oxo-4-(o-tolyl)-1,2-dihydroisoquinolin-7-yl)propanamide (Example 47, 100 mg). LCMS (ESI) Calcd. for C₂₁H₂₃N₃O₂: 349, found [M+H]⁺=350. ¹H NMR (400 MHz, DMSO-d₆) δ 11.04 (s, 1H), 7.32 (s, 2H), 7.27-7.23 (m, 2H), 7.16 (d, 1H), 7.02 (m, 1H), 6.72 (d, 1H), 6.66 (d, 1H), 6.20 (d, 1H), 4.52 (m, 1H), 3.14 (s, 3H), 2.84 (s, 3H), 2.04 (s, 3H), 1.28 (d, 3H).

### Example 48: Synthesis of 7-phenyl-4-(o-tolyl)isoquinolin-1(2H)-one

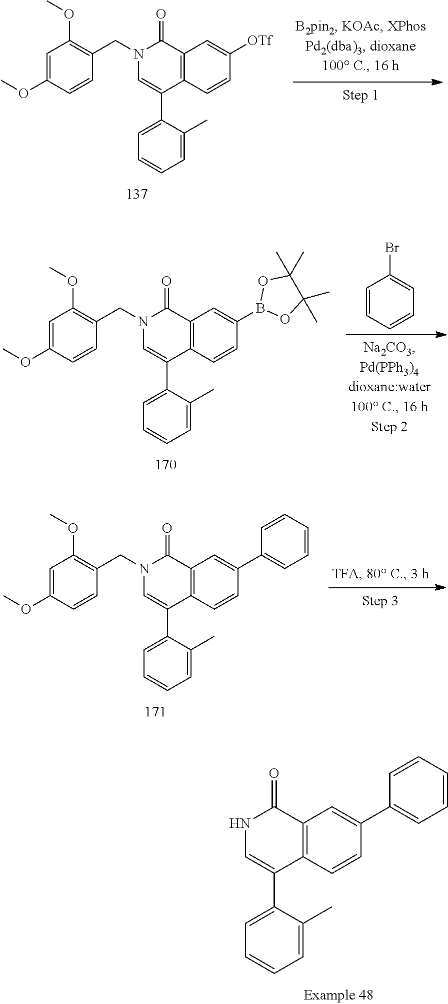

Synthesis of 2-(2,4-dimethoxybenzyl)-7-(4,4,5,5-tetramethyl-1,3,2-dioxaborolan-2-yl)-4-(o-tolyl)isoquinolin-1(2H)-one, 170 [Step 1]: In a 50 mL two-neck round bottom flask, potassium acetate (140 mg, 1.4 mmol) was dried under argon atmosphere using a heat gun for 15 min. After cooling, 1,4-dioxane (5 mL), 2-(2,4-dimethoxybenzyl)-1-oxo-4-(o-tolyl)-1,2-dihydroisoquinolin-7-yl trifluoromethanesulfonate (137, 250 mg, 0.5 mmol), and B₂pin₂ (155 mg, 0.6 mmol) were added to the reaction flask. The reaction mixture was purged with argon for 5 min. To the reaction mixture was added XPhos (22 mg, 0.05 mmol) and Pd₂(dba)₃ (20 mg, 0.02 mmol). The reaction mixture was heated at 100° C. for 16 h. The reaction mixture was filtered through celite and concentrated under reduced pressure. The product was purified by flash chromatography to afford 2-(2,4-dimethoxybenzyl)-7-(4,4,5,5-tetramethyl-1,3,2-dioxaborolan-2-yl)-4-(o-tolyl)isoquinolin-1(2H)-one (170, 110 mg). LCMS (ESI) Calcd. for C₃₁H₃₄BNO₅: 511, found [M+H]⁺: 513.

Synthesis of 2-(2,4-dimethoxybenzyl)-7-phenyl-4-(o-tolyl)isoquinolin-1(2H)-one, 171 [Step 2]: To a stirred solution of 2-(2,4-dimethoxybenzyl)-7-(4,4,5,5-tetramethyl-1,3,2-dioxaborolan-2-yl)-4-(o-tolyl)isoquinolin-1(2H)-one (170, 100 mg, 0.2 mmol) in 1,4-dioxane (4 mL) and water (1 mL) was added bromobenzene (45 mg, 0.3 mmol) and Na₂CO₃ (125 mg, 0.4 mmol). The reaction mixture was purged with argon for 10 min. Pd(PPh₃)₄ (25 mg, 0.02 mmol) was added, and the reaction mixture was allowed to stir for 16 h. at 100° C. The reaction mixture was filtered through celite. The filtrate was diluted with EtOAc and washed with water, brine. The organic phase was dried over anhydrous Na₂SO₄, filtered, and concentrated under reduced pressure. The product was purified by flash chromatography to afford 2-(2,4-dimethoxybenzyl)-7-phenyl-4-(o-tolyl)isoquinolin-1(2H)-one (171, 40 mg). LCMS (ESI) Calcd. for C₃₁H₂₇NO₃: 461, found [M+H]⁺=462. ¹H NMR (400 MHz, DMSO-d₆) δ 8.54 (s, 1H), 7.99-7.96 (d, 1H), 7.74 (d, 2H), 7.52-7.48 (m, 2H), 7.42-7.29 (m, 5H), 7.24 (d, 1H), 7.11 (m, 2H), 6.59 (s, 1H), 6.50 (d, 1H), 5.11 (m, 2H), 3.79-3.73 (s, 6H), 2.08 (s, 3H).

Synthesis of 7-phenyl-4-(o-tolyl)isoquinolin-1(2H)-one, Example 48 [Step 3]: A solution of 2-(2,4-dimethoxybenzyl)-7-phenyl-4-(o-tolyl)isoquinolin-1(2H)-one (171, 40 mg, 0.09 mmol) and TFA (0.13 mL, 1.7 mmol) was heated at 80° C. for 4 h. The reaction mixture was concentrated under reduced pressure, basified by an aq. NaHCO₃ solution, and extracted with EtOAc. The combined organic layers were dried over anhydrous Na₂SO₄, filtered, and concentrated under reduced pressure. The product was purified by reverse phase prep-HPLC to afford 7-phenyl-4-(o-tolyl)isoquinolin-1(2H)-one (Example 48, 12 mg). LCMS (ESI) Calcd. for C₂₂H₁₇NO: 311, found [M+H]⁺=312. ¹H NMR (400 MHz, DMSO-d₆) δ 11.45 (s, 1H), 8.51 (d, 1H), 7.98-7.96 (d, 1H), 7.75 (d, 2H), 7.52-7.48 (t, 2H), 7.42-7.37 (m, 3H), 7.33-7.30 (m, 1H), 7.25 (d, 1H), 7.07-7.03 (m, 2H), 2.08 (s, 3H).

### Examples 49-50: Synthesis of chiral 2-((4-(2-chloro-4-fluorophenyl)-1-oxo-1,2-dihydroisoquinolin-7-yl)oxy)propanenitrile

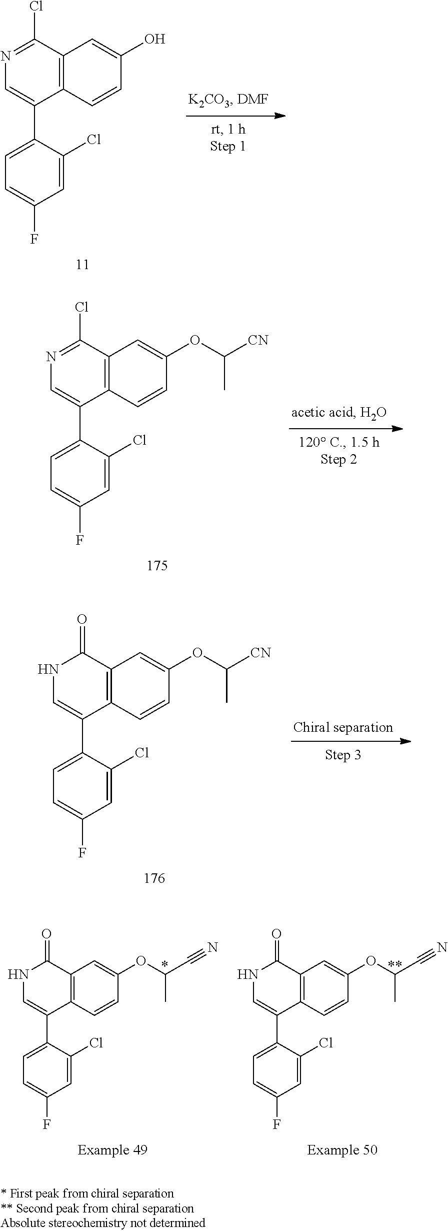 [SMILES: Oc1ccc2c(-c3ccc(F)cc3Cl)cnc(Cl)c2c1 ; CC(C#N)Oc1ccc2c(-c3ccc(F)cc3Cl)cnc(Cl)c2c1 ; CC(C#N)Oc1ccc2c(-c3ccc(F)cc3Cl)c[nH]c(=O)c2c1 ; CC(C#N)Oc1ccc2c(-c3ccc(F)cc3Cl)c[nH]c(=O)c2c1 ; CC(C#N)Oc1ccc2c(-c3ccc(F)cc3Cl)c[nH]c(=O)c2c1]

Synthesis of 2-((1-chloro-4-(2-chloro-4-fluorophenyl)isoquinolin-7-yl)oxy)propanenitrile, 175 [Step 1]: To a stirred solution of 1-chloro-4-(2-chloro-4-fluoro-phenyl)isoquinolin-7-ol (11, 50 mg, 0.2 mmol) in DMF (5 mL) was added K₂CO₃ (90 mg, 0.7 mmol) followed by 2-bromopropanenitrile (0.028 mL, 0.3 mmol). The reaction mixture was stirred at ambient temperature for 2 h. The reaction mixture was partitioned between EtOAc and water. The combined organic layers were washed with water, brine, dried over anhydrous Na₂SO₄, filtered, and concentrated under reduced pressure. The product was purified by flash column chromatography to afford 2-[[1-chloro-4-(2-chloro-4-fluoro-phenyl)-7-isoquinolyl]oxy]propanenitrile (175, 40 mg). ¹H NMR (400 MHz, DMSO-d₆) δ 8.18 (s, 1H), 7.86 (d, 1H), 7.72 (d, 1H), 7.65-7.57 (m, 2H), 7.49-7.42 (m, 2H), 5.84-5.82 (m, 1H), 1.78 (d, 3H). LCMS (ESI) Calcd. for C₁₈H₁₁Cl₂FN₂O: 361, found [M+1]⁺=361, 363, 364.

Synthesis of 2-((4-(2-chloro-4-fluorophenyl)-1-oxo-1,2-dihydroisoquinolin-7-yl)oxy)propanenitrile, 176 [Step-2]: To a stirred solution of 2-[[1-chloro-4-(2-chloro-4-fluoro-phenyl)-7-isoquinolyl]oxy]propanenitrile (175, 220 mg, 0.6 mmol) in acetic acid (5.2 mL, 91.4 mmol) was added water (1.1 mL, 60.9 mmol) at ambient temperature. The reaction mixture was heated to reflux for 16 h. The reaction mixture was diluted with water and extracted with EtOAc. The organic layer was washed with brine, dried over anhydrous Na₂SO₄, filtered, and concentrated under reduced pressure. The product was purified by flash chromatography to afford 2-((4-(2-chloro-4-fluorophenyl)-1-oxo-1,2-dihydroisoquinolin-7-yl)oxy)propanenitrile (176, 152 mg).

Synthesis of chiral 2-((4-(2-chloro-4-fluorophenyl)-1-oxo-1,2-dihydroisoquinolin-7-yl)oxy)propanenitrile, Examples 49 and 50 [Step 3]: 2-((4-(2-chloro-4-fluorophenyl)-1-oxo-1,2-dihydroisoquinolin-7-yl)oxy)propanenitrile (176, 152 mg) was purified by SFC chiral prep-HPLC and lyophilized. The first product was isolated as 2-((4-(2-chloro-4-fluorophenyl)-1-oxo-1,2-dihydroisoquinolin-7-yl)oxy)propanenitrile, Peak 1 (Example 49, 25 mg) and the second product as 2-((4-(2-chloro-4-fluorophenyl)-1-oxo-1,2-dihydroisoquinolin-7-yl)oxy)propanenitrile, Peak 2 (Example 50, 50 mg). The absolute stereochemistry for these Examples was not determined.

Example 49: 2-((4-(2-chloro-4-fluorophenyl)-1-oxo-1,2-dihydroisoquinolin-7-yl)oxy)propanenitrile, Peak 1: LCMS (ESI) Calcd. for C₁₈H₁₂ClFN₂O₂: 343, found [M+H]⁺=343, 345, 685, 687. ¹H NMR (400 MHz, DMSO-d₆) δ 11.58 (s, 1H), 7.89 (t, 1H), 7.63-7.61 (m, 1H), 7.52-7.48 (m, 1H), 7.41-7.32 (m, 2H), 7.08 (s, 1H), 7.04-7.01 (dd, 1H), 5.66-5.64 (q, 1H), 1.72 (d, 3H).

Example 50: 2-((4-(2-chloro-4-fluorophenyl)-1-oxo-1,2-dihydroisoquinolin-7-yl)oxy)propanenitrile, Peak 2: LCMS (ESI) Calcd. for C₁₈H₁₂ClFN₂O₂: 343, found [M+H]⁺=343. ¹H NMR (400 MHz, DMSO-d₆) δ 11.58 (s, 1H), 7.89 (t, 1H), 7.64-7.61 (m, 1H), 7.52-7.48 (m, 1H), 7.41-7.35 (m, 2H), 7.08 (s, 1H), 7.04-7.01 (dd, 1H), 5.66-5.64 (q, 1H), 1.73 (d, 3H).

Chiral SFC: Chiral separation was performed on a Thar SFC-80 series instrument. Column was a Regis Reflect (R,R) whelk-01 (21×250 mm), 5 μm, operating at 35° C. with flow rate of 50 g/min. Mobile Phase: 70% CO₂ and 30% MeOH/hexanes/IPA/CH₂Cl₂ (70/20/5/5) at 100 bar with detection at 220 nm wavelength. Diluent: MeOH and CH₂Cl₂ excess. Sample concentration: 12.4 mg/ml. Loading: 6.5 mg/4 min.

### Examples 51-53: Synthesis of 3-methoxy-2-((1-oxo-4-(o-tolyl)-1,2-dihydroisoquinolin-7-yl)oxy)propanoic acid and chiral 3-methoxy-2-((1-oxo-4-(o-tolyl)-1,2-dihydroisoquinolin-7-yl)oxy)propenamide

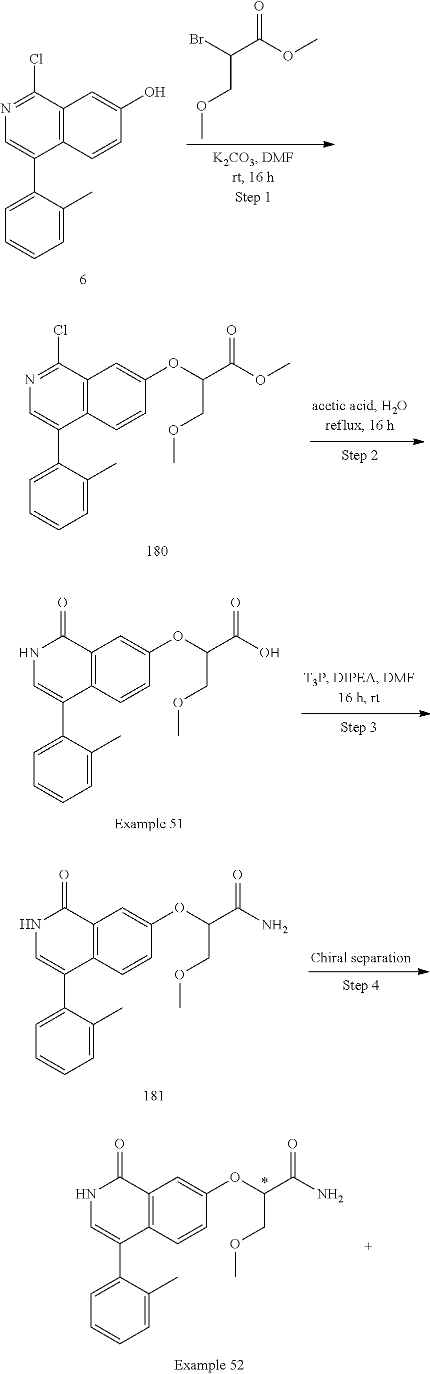

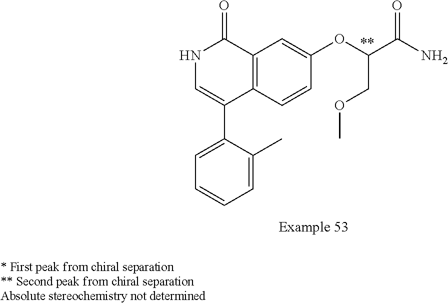 [SMILES: COCC(Oc1ccc2c(-c3ccccc3C)c[nH]c(=O)c2c1)C(N)=O]

Synthesis methyl 2-((1-chloro-4-(o-tolyl)isoquinolin-7-yl)oxy)-3-methoxypropanoate, 180 [Step 1]: To a stirred solution of 1-chloro-4-(o-tolyl)isoquinolin-7-ol (6, 200 mg, 0.7 mmol) in DMF (3 mL) was added K₂CO₃ (256 mg, 1.8 mmol) and methyl 2-bromo-3-methoxy-propanoate (175 mg, 0.9 mmol). The reaction mixture was stirred at ambient temperature for 16 h. The reaction mixture was quenched with ice cold water and extracted with EtOAc. The organic layer was washed with brine, dried over anhydrous Na₂SO₄, filtered, and concentrated under reduced pressure. The product was purified by flash chromatography to afford methyl 2-((1-chloro-4-(o-tolyl)isoquinolin-7-yl)oxy)-3-methoxypropanoate (180, 250 mg). LCMS (ESI) Calcd. for C₂₁H₂₀ClNO₄: 385, found [M+H]⁺=386. ¹H NMR (400 MHz, DMSO-d₆) δ 8.06 (s, 1H), 7.57-7.55 (m, 2H), 7.45-7.42 (m, 2H), 7.35-7.30 (m, 2H), 7.26-7.23 (m, 1H), 5.46 (m, 1H), 3.97-3.81 (m, 2H), 3.72 (d, 3H), 3.35 (s, 3H), 1.96 (s, 3H).

Synthesis of 3-methoxy-2-((1-oxo-4-(o-tolyl)-1,2-dihydroisoquinolin-7-yl)oxy)propanoic acid, Example 51 [Step 2]: To a stirred solution of 2-((1-chloro-4-(o-tolyl)isoquinolin-7-yl)oxy)-3-methoxypropanoate (180, 300 mg, 0.8 mmol) in acetic acid (5.1 mL, 89.4 mmol) was added water (1.4 mL, 77.8 mmol). The reaction mixture was heated to reflux for 16 h. The reaction mixture was concentrated under reduced pressure and extracted in EtOAc. The combined organic layers were washed with water, brine, dried over anhydrous Na₂SO₄, filtered, and concentrated under reduced pressure. The product was purified by reverse phase prep-HPLC and lyophilized to afford 3-methoxy-2-((1-oxo-4-(o-tolyl)-1,2-dihydroisoquinolin-7-yl)oxy)propanoic acid (Example 51, 200 mg). LCMS (ESI) Calcd. for C₂₀H₁₉NO₅: 353, found [M+H]⁺=354. ¹H NMR (400 MHz, DMSO-d₆) δ 11.30 (s, 1H), 7.56 (m, 1H), 7.40 (s, 1H), 7.34 (m, 2H), 7.29-7.25 (m, 1H), 7.23-7.17 (m, 2H), 6.87-6.85 (d, 2H), 4.70 (m, 1H), 3.77-3.70 (m, 2H), 3.33 (s, 3H), 2.04 (s, 3H).

Synthesis of 3-methoxy-2-((1-oxo-4-(o-tolyl)-1,2-dihydroisoquinolin-7-yl)oxy)propenamide, 181 [Step 3]: To a stirred solution of 3-methoxy-2-[[4-(o-tolyl)-1-oxo-2H-isoquinolin-7-yl]oxy]propanoic acid (Example 51, 200 mg, 0.6 mmol) in DMF (3 mL) was added (NH₄)₂CO₃ (272 mg, 2.8 mmol) and DIPEA (0.5 mL, 2.8 mmol). T₃P (0.5 mL, 0.9 mmol, 50% in EtOAc) was added at 0° C. The reaction mixture was warmed to ambient temperature and stirred for 16 h. The reaction mixture was diluted with ice cold water and extracted with EtOAc. The combined organic phase was washed with brine, dried over anhydrous Na₂SO₄, filtered, and concentrated under reduced pressure. The product was purified by flash chromatography to afford the 3-methoxy-2-((1-oxo-4-(o-tolyl)-1,2-dihydroisoquinolin-7-yl)oxy)propanamide (181, 95 mg). LCMS (ESI) Calcd. for C₂₀H₂₀N₂O₄: 352, found [M+H]⁺=353.

Synthesis of chiral 3-methoxy-2-((1-oxo-4-(o-tolyl)-1,2-dihydroisoquinolin-7-yl)oxy)propenamide, Examples 52 and 53 [Step 4]: 3-methoxy-2-((1-oxo-4-(o-tolyl)-1,2-dihydroisoquinolin-7-yl)oxy)propanamide was purified by normal phase chiral prep-HPLC and lyophilized. The first product was isolated as 3-methoxy-2-((1-oxo-4-(o-tolyl)-1,2-dihydroisoquinolin-7-yl)oxy)propenamide, Peak 1 (Example 52, 18 mg) and the second product as 3-methoxy-2-((1-oxo-4-(o-tolyl)-1,2-dihydroisoquinolin-7-yl)oxy)propenamide, Peak 2 (Example 53, 25 mg). The absolute stereochemistry for these Examples was not determined.

Example 52: 3-methoxy-2-((1-oxo-4-(o-tolyl)-1,2-dihydroisoquinolin-7-yl)oxy)propenamide, Peak 1: LCMS (ESI) Calcd. for C₂₀H₂₀N₂O₄: 352, found [M+H]⁺=353. ¹H NMR (400 MHz, DMSO-d₆) δ 11.36 (d, 1H), 7.70-7.68 (m, 2H), 7.40 (s, 1H), 7.35-7.24 (m, 4H), 7.19 (d, 1H), 6.92-6.88 (m, 2H), 4.82 (dd, 1H), 3.78-3.69 (m, 2H), 2.04 (d, 3H).

Example 53: 3-methoxy-2-((1-oxo-4-(o-tolyl)-1,2-dihydroisoquinolin-7-yl)oxy)propenamide, Peak 2: LCMS (ESI) Calcd. for C₂₀H₂₀N₂O₄: 352, found [M+H]⁺=353. ¹H NMR (400 MHz, DMSO-d₆) δ 11.36 (s, 1H), 7.70-7.68 (m, 2H), 7.40 (s, 1H), 7.35-7.32 (m, 2H), 7.31-7.27 (m, 1H), 7.19 (d, 1H), 6.92-6.89 (m, 2H), 4.82 (m, 1H), 3.78-3.68 (m, 2H), 2.04 (s, 3H).

Chiral prep-HPLC: Chiral separation was performed on an Agilent 1200 series instrument. Column was a Chiralpak IG (250×21 mm), 5 μm, operating at ambient temperature with flow rate of 21.0 mL/min. Mobile phase: 0.1% isopropylamine in a mixture of 50% hexane, 25% ethanol, and 25% EtOAc, held isocratic for up to 25 min. with detection at 224 nm wavelength.

### Examples 54-55: Synthesis of chiral 3-methoxy-N,N-dimethyl-2-((1-oxo-4-(o-tolyl)-1,2-dihydroisoquinolin-7-yl)oxy)propenamide

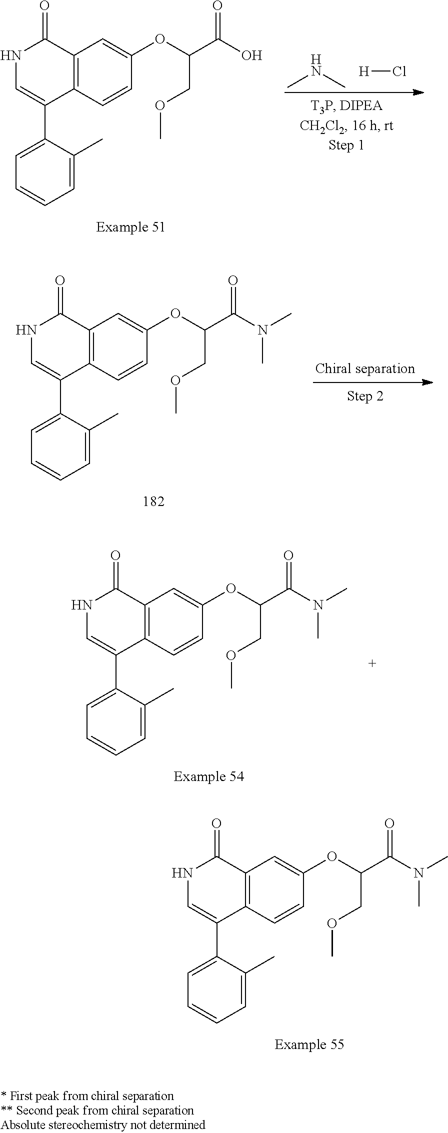

Synthesis of 3-methoxy-N,N-dimethyl-2-((1-oxo-4-(o-tolyl)-1,2-dihydroisoquinolin-7-yl)oxy)propenamide, 182 [Step 1]: To a stirred solution of 3-methoxy-2-((1-oxo-4-(o-tolyl)-1,2-dihydroisoquinolin-7-yl)oxy)propanoic acid (Example 51, 150 mg, 0.4 mmol) in CH₂Cl₂ (3 mL) was added N,N-dimethylamine.HCl (173 mg, 2.1 mmol), DIPEA (0.5 mL, 2.6 mmol), and T₃P (0.4 mL, 0.7 mmol). The reaction mixture was stirred at ambient temperature for 16 h. The reaction mixture was concentrated under reduced pressure. The reaction mixture was diluted with water and extracted with EtOAc. The organic phase was washed with brine, dried over anhydrous Na₂SO₄, filtered, and concentrated under reduced pressure. The product was purified by reverse phase prep-HPLC to afford 3-methoxy-N,N-dimethyl-2-((1-oxo-4-(o-tolyl)-1,2-dihydroisoquinolin-7-yl)oxy)propanamide (182, 80 mg). LCMS (ESI) Calcd. for C₂₂H₂₄N₂O₄: 380, found [M+H]⁺=381.

Synthesis of chiral 3-methoxy-N,N-dimethyl-2-((1-oxo-4-(o-tolyl)-1,2-dihydroisoquinolin-7-yl)oxy)propenamide, Examples 54 and 55 [Step 2]: 3-methoxy-N,N-dimethyl-2-((1-oxo-4-(o-tolyl)-1,2-dihydroisoquinolin-7-yl)oxy)propanamide was purified by normal phase chiral prep-HPLC and lyophilized. The first product was isolated as 3-methoxy-N,N-dimethyl-2-((1-oxo-4-(o-tolyl)-1,2-dihydroisoquinolin-7-yl)oxy) propenamide, Peak 1 (Example 54, 40 mg) and the second product as 3-methoxy-N,N-dimethyl-2-((1-oxo-4-(o-tolyl)-1,2-dihydroisoquinolin-7-yl)oxy) propenamide, Peak 2 (Example 55, 45 mg). The absolute stereochemistry for these Examples was not determined.

Example 54: 3-methoxy-N,N-dimethyl-2-((1-oxo-4-(o-tolyl)-1,2-dihydroisoquinolin-7-yl)oxy) propenamide, Peak 1: LCMS (ESI) Calcd. for C₂₂H₂₄N₂O₄: 380, found [M+H]⁺=381. ¹H NMR (400 MHz, DMSO-d₆) δ 11.34 (d, 1H), 7.58 (dd, 1H), 7.34 (d, 2H), 7.33-7.24 (m, 2H), 7.19 (d, 1H), 6.91 (t, 2H), 5.43 (q, 1H), 3.74 (d, 2H), 3.33 (s, 3H), 3.15 (d, 3H), 2.84 (s, 3H), 2.04 (s, 3H).

Example 55: 3-methoxy-N,N-dimethyl-2-((1-oxo-4-(o-tolyl)-1,2-dihydroisoquinolin-7-yl)oxy)propenamide, Peak 2: LCMS (ESI) Calcd. for C₂₂H₂₄N₂O₄: 380, found [M+H]⁺=381. ¹H NMR (400 MHz, DMSO-d₆) δ 11.34 (d, 1H), 7.58 (dd, 1H), 7.34 (d, 2H), 7.33-7.22 (m, 2H), 7.19 (d, 1H), 6.89 (t, 2H), 5.47-5.38 (m, 1H), 3.75 (d, 2H), 3.33 (s, 3H), 3.15 (d, 3H), 2.84 (s, 3H), 2.04 (s, 3H).

Chiral SFC: Chiral separation was performed on a Thar SFC-80 series instrument. Column was a Regis Reflect (R,R) whelk-01 (250×21.1 mm), 5 μm, operating at 35° C. temperature with flow rate of 60 μm/min. Mobile phase: 60% CO₂ in super critical state and 40% of 0.5% isopropylamine in IPA, held isocratic for up to 12 min. at 100 bar with detection at 220 nm wavelength.

### Examples 56-58: Synthesis of 4-(2-chlorophenyl)-7-methoxyisoquinolin-1(2H)-one and chiral 2-((4-(2-chlorophenyl)-1-oxo-1,2-dihydroisoquinolin-7-yl)oxy)propanenitrile

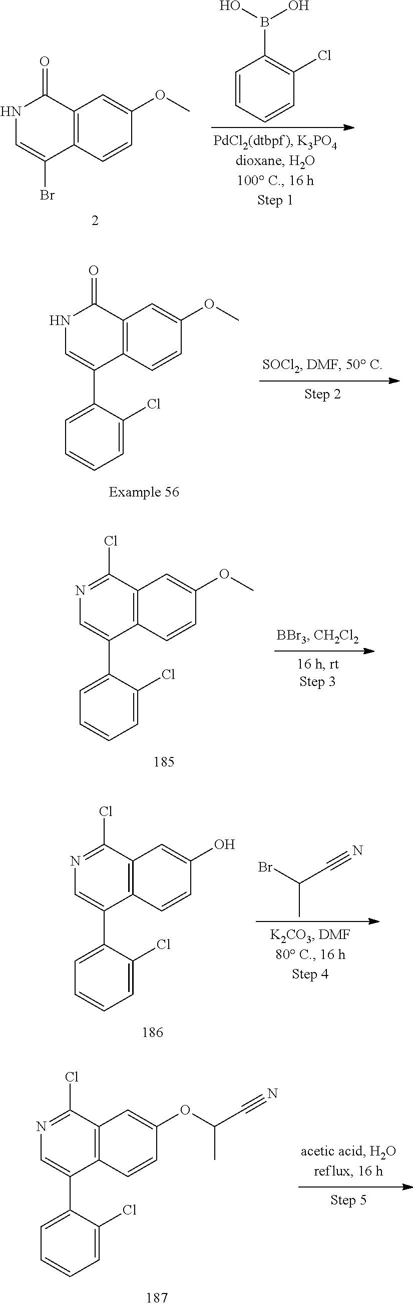

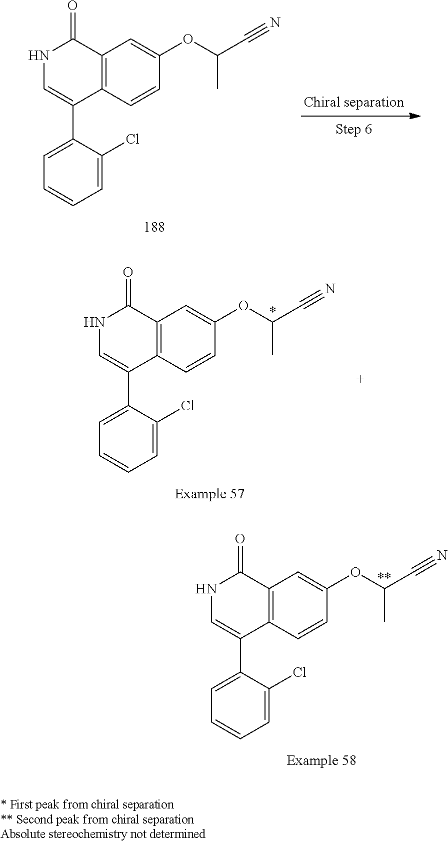 [SMILES: CC(C#N)Oc1ccc2c(-c3ccccc3Cl)c[nH]c(=O)c2c1 ; CC(C#N)Oc1ccc2c(-c3ccccc3Cl)c[nH]c(=O)c2c1 ; CC(C#N)Oc1ccc2c(-c3ccccc3Cl)c[nH]c(=O)c2c1]

Synthesis of 4-(2-chlorophenyl)-7-methoxyisoquinolin-1(2H)-one, Example 56 [Step 1]: To a stirred solution of 4-bromo-7-methoxy-isoquinolin-1(2H)-one (2, 4 g, 15.7 mmol) in 1,4-dioxane (70 mL) and water (15 mL) was added K₃PO₄ (8.35 g, 39.4 mmol) and (2-chlorophenyl)boronic acid (3.69 g, 23.6 mmol). The reaction mixture was purged with argon for 5 min., and PdCl₂(dtbpf) (1.03 g, 1.6 mmol) was added. The reaction mixture was heated at 100° C. for 16 h. The reaction mixture was filtered through celite. The filtrate was diluted with EtOAc, washed with water, dried over anhydrous Na₂SO₄, filtered, and concentrated under reduced pressure. The product was purified by flash chromatography to afford 4-(2-chlorophenyl)-7-methoxyisoquinolin-1(2H)-one (Example 56, 2 g). LCMS (ESI) Calcd. for C₁₆H₁₂ClNO₂: 285, found [M+H]⁺=286.

Synthesis of 1-chloro-4-(2-chlorophenyl)-7-methoxyisoquinoline, 185 [Step 2]: To a stirred solution of 4-(2-chlorophenyl)-7-methoxyisoquinolin-1(2H)-one (Example 56, 600 mg, 2.1 mmol) in SOCl₂ (7.6 mL, 105 mmol) was added DMF (0.2 mL, 2.1 mmol) under an argon atmosphere. The reaction mixture was heated at 50° C. for 16 h. The reaction mixture was concentrated under reduced pressure, diluted with ice cold water, and extracted with EtOAc. The combined organic layers were washed with brine, dried over anhydrous Na₂SO₄, filtered, and concentrated under reduced pressure. The product was purified by flash chromatography to afford 1-chloro-4-(2-chlorophenyl)-7-methoxyisoquinoline (185, 550 mg). LCMS (ESI) Calcd. for C₁₆H₁₁Cl₂NO: 303, found [M+H]⁺=304. ¹H NMR (400 MHz, DMSO-d₆) δ 8.10 (s, 1H), 7.69 (d, 1H), 7.64-7.44 (m, 5H), 7.36 (d, 1H), 3.99 (s, 3H).

Synthesis of 1-chloro-4-(2-chlorophenyl)isoquinolin-7-ol, 186 [Step 3]: To a stirred solution of 1-chloro-4-(2-chlorophenyl)-7-methoxyisoquinoline (185, 560 mg, 1.9 mmol) in CH₂Cl₂ (6 mL) was added dropwise BBr₃ (5.5 mL, 5.5 mmol, 1M in CH₂Cl₂) at 0° C. The reaction mixture was stirred at ambient temperature for 16 h. The reaction mixture was concentrated under reduced pressure, cooled to 0° C., quenched with ice water and MeOH, and extracted with EtOAc. The combined organic layers were washed with brine, dried over anhydrous Na₂SO₄, filtered, and concentrated under reduced pressure. The product was purified by flash chromatography to afford 1-chloro-4-(2-chlorophenyl)isoquinolin-7-ol (186, 430 mg). LCMS (ESI) Calcd. For C₁₅H₉Cl₂NO: 289; found [M+H]⁺=290. ¹H NMR (400 MHz, DMSO-d₆) δ 10.66 (s, 1H), 7.99 (s, 1H), 7.68 (dd, 1H), 7.59-7.44 (m, 4H), 7.41 (dd, 1H), 7.31 (d, 1H).

Synthesis of 2-((1-chloro-4-(2-chlorophenyl)isoquinolin-7-yl)oxy)propanenitrile, 187 [Step 4]: To a stirred solution of 1-chloro-4-(2-chlorophenyl)isoquinolin-7-ol (186, 200 mg, 0.7 mmol) in DMF (3 mL) was added K₂CO₃ (256 mg, 1.9 mmol) and 2-bromopropanenitrile (120 mg, 0.9 mmol). The reaction mixture was stirred at 80° C. for 2 h. The reaction mixture was quenched with ice cold water and extracted with EtOAc. The combined organic layers were washed with brine, dried over anhydrous Na₂SO₄, filtered, concentrated under reduced pressure. The product was purified by flash chromatography to afford 2-((1-chloro-4-(2-chlorophenyl)isoquinolin-7-yl)oxy)propanenitrile (187, 200 mg). LCMS (ESI) Calcd. for C₁₈H₁₂Cl₂N₂O: 342, found [M+H]⁺=343.

Synthesis of 2-((4-(2-chlorophenyl)-1-oxo-1,2-dihydroisoquinolin-7-yl)oxy)propanenitrile, 188 [Step 5]: To a solution of 2-((1-chloro-4-(2-chlorophenyl)isoquinolin-7-yl)oxy)propanenitrile (187, 170 mg, 0.5 mmol) in acetic acid (3.3 mL, 57 mmol) was added water (0.9 mL, 49.5 mmol). The reaction mixture was stirred at 120° C. for 16 h. The reaction mixture was concentrated under reduced pressure, diluted with water, and extracted with EtOAc. The combined organic layers were washed with brine, dried over anhydrous Na₂SO₄, filtered, and concentrated under reduced pressure. The product was purified by reverse phase prep-HPLC to afford 2-((4-(2-chlorophenyl)-1-oxo-1,2-dihydroisoquinolin-7-yl)oxy)propanenitrile (188, 130 mg). LCMS (ESI) Calcd. for C₁₈H₁₃ClN₂O₂: 325, found [M+H]⁺=325.

Synthesis of chiral 2-((4-(2-chlorophenyl)-1-oxo-1,2-dihydroisoquinolin-7-yl)oxy)propanenitrile, Examples 57 and 58 [Step-6]: 2-((4-(2-chlorophenyl)-1-oxo-1,2-dihydroisoquinolin-7-yl)oxy)propanenitrile (188, 130 mg, 0.4 mmol) was purified by normal phase chiral prep-HPLC and lyophilized. The first product was isolated as 2-((4-(2-chlorophenyl)-1-oxo-1,2-dihydroisoquinolin-7-yl)oxy)propanenitrile, Peak 1 (Example 57, 70 mg) and the second product as 2-((4-(2-chlorophenyl)-1-oxo-1,2-dihydroisoquinolin-7-yl)oxy)propanenitrile, Peak 2 (Example 58, 55 mg). The absolute stereochemistry for these Examples was not determined.

Example 57: 2-((4-(2-chlorophenyl)-1-oxo-1,2-dihydroisoquinolin-7-yl)oxy)propanenitrile, Peak 1: LCMS (ESI) Calcd. for C₁₈H₁₃ClN₂O₂: 325, found [M−H]⁻=323. ¹H NMR (400 MHz, DMSO-d₆) δ 11.57 (d, 1H), 7.90 (t, 1H), 7.66-7.57 (m, 1H), 7.55-7.41 (m, 3H), 7.45-7.35 (m, 1H), 7.07 (d, 1H), 7.02 (dd, 1H), 5.71-5.60 (m, 1H), 1.72 (d, 3H).

Example 58: 2-((4-(2-chlorophenyl)-1-oxo-1,2-dihydroisoquinolin-7-yl)oxy)propanenitrile, Peak 2: LCMS (ESI) Calcd. for C₁₈H₁₃ClN₂O₂: 325, found [M−H]⁻=323. ¹H NMR (400 MHz, DMSO-d₆) δ 11.57 (d, 1H), 7.90 (s, 1H), 7.61 (d, 1H), 7.53-7.43 (m, 3H), 7.44-7.35 (m, 1H), 7.07 (d, 1H), 7.01 (d, 1H), 5.65 (q, 1H), 1.72 (d, 3H).

Chiral prep-HPLC: Chiral separation was performed on an Agilent 1200 series instrument. Column was a Chiralpak IC (250×20 mm), 5 μm, operating at ambient temperature with flow rate of 18.0 mL/min. Mobile phase: mixture of 10% ethanol, 10% EtOAc, and 80% hexane, held isocratic for up to 15 min. with detection at 282 nm wavelength.

### Example 59: Synthesis of (S)-1-((S)-2-((4-(2-chloro-4-fluorophenyl)-1-oxo-1,2-dihydroisoquinolin-7-yl)oxy)propanoyl)piperidine-3-carboxylic acid

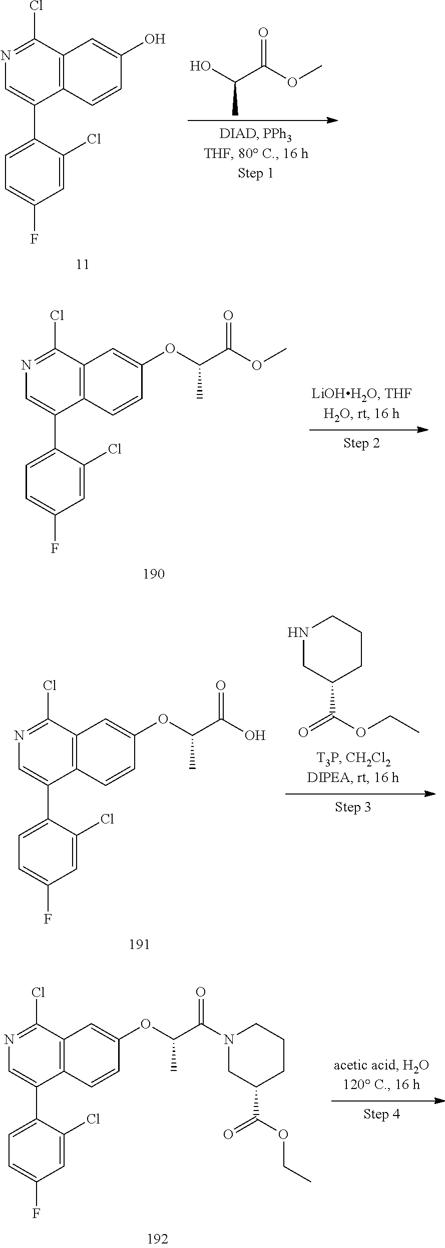

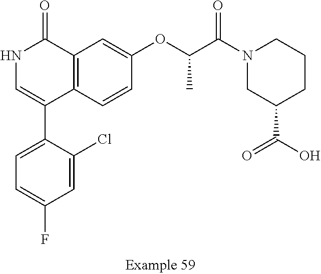 [SMILES: C[C@H](Oc1ccc2c(-c3ccc(F)cc3Cl)c[nH]c(=O)c2c1)C(=O)N1CCC[C@H](C(=O)O)C1]

Synthesis of methyl (S)-2-((1-chloro-4-(2-chloro-4-fluorophenyl)isoquinolin-7-yl)oxy)propanoate, 190 [Step 1]: To a stirred solution of 1-chloro-4-(2-chloro-4-fluorophenyl)isoquinolin-7-ol (11, 300 mg, 1.0 mmol) and methyl (R)-2-hydroxypropanoate (0.2 mL, 1.9 mmol) in THF (10 mL) was added PPh₃ (766 mg, 2.9 mmol). DIAD (0.5 mL, 2.9 mmol) was added at 0° C. The reaction mixture was allowed to warm to ambient temperature, then stirred at 80° C. for 16 h. The reaction mixture was diluted with water, extracted with EtOAc, washed with brine, dried over anhydrous Na₂SO₄, and concentrated under reduced pressure. The product was purified by flash chromatography to afford methyl (S)-2-((1-chloro-4-(2-chloro-4-fluorophenyl)isoquinolin-7-yl)oxy)propanoate (190, 200 mg). LCMS (ESI) Calcd. for C₁₉H₁₄Cl₂FNO₃: 393, found [M+H]⁺=394.

Synthesis of (S)-2-((1-chloro-4-(2-chloro-4-fluorophenyl) isoquinolin-7-yl)oxy) propanoic acid, 191 [Step 2]: To a stirred solution of methyl (S)-2-((1-chloro-4-(2-chloro-4-fluorophenyl)isoquinolin-7-yl)oxy)propanoate (190, 140 mg, 0.4 mmol) in THF (4 mL) was added dropwise LiOH·H₂O (30 mg, 0.7 mmol) dissolved in water (1 mL). The reaction mixture was stirred for 2 h. at ambient temperature. The reaction mixture was concentrated under reduced pressure, diluted with water, acidified with citric acid, and extracted with EtOAc. The combined organic layers were washed with brine, dried over anhydrous Na₂SO₄, filtered, and concentrated under reduced pressure to afford (S)-2-((1-chloro-4-(2-chloro-4-fluorophenyl)isoquinolin-7-yl)oxy)propanoic acid (191, 120 mg). LCMS (ESI) Calcd. for C₁₈H₁₂Cl₂FNO₃: 379, found [M+H]⁺=380.

Synthesis of ethyl (S)-1-((S)-2-((1-chloro-4-(2-chloro-4-fluorophenyl)isoquinolin-7-yl)oxy)propanoyl)piperidine-3-carboxylate, 192 [Step 3]: To a stirred solution of (S)-2-((1-chloro-4-(2-chloro-4-fluorophenyl)isoquinolin-7-yl)oxy)propanoic acid (191, 110 mg, 0.3 mmol) in CH₂Cl₂ (3 mL) was added ethyl (S)-piperidine-3-carboxylate (136 mg, 0.9 mmol), DIPEA (0.5 mL, 2.8 mmol) and T₃P (0.2 mL, 0.3 mmol). The reaction mixture was stirred at ambient temperature for 16 h. The reaction mixture was quenched with cold water and extracted with CH₂Cl₂. The combined organic layers were washed with brine, dried over anhydrous Na₂SO₄, filtered, and concentrated under reduced pressure. The product was purified by flash chromatography to afford ethyl (S)-1-((S)-2-((1-chloro-4-(2-chloro-4-fluorophenyl)isoquinolin-7-yl)oxy)propanoyl)piperidine-3-carboxylate (192, 130 mg). LCMS (ESI) Calcd. for C₂₆H₂₅Cl₂FN₂O₄: 518, found [M+H]⁺=519.

Synthesis of (S)-1-((S)-2-((4-(2-chloro-4-fluorophenyl)-1-oxo-1,2-dihydroisoquinolin-7-yl)oxy)propanoyl)piperidine-3-carboxylic acid, Example 59 [Step 4]: To a solution of ethyl (S)-1-((S)-2-((1-chloro-4-(2-chloro-4-fluorophenyl)isoquinolin-7-yl)oxy)propanoyl)piperidine-3-carboxylate (191, 120 mg, 0.2 mmol) in acetic acid (1.5 mL, 27 mmol) was added water (0.4 mL, 23 mmol). The reaction mixture was heated at 120° C. for 16 h. The reaction mixture was concentrated under reduced pressure, diluted with water, and extracted with EtOAc. The combined organic layers were washed with brine, dried over anhydrous Na₂SO₄, filtered, and concentrated under reduced pressure. The product was purified by reverse phase prep-HPLC to afford (S)-1-((S)-2-((4-(2-chloro-4-fluorophenyl)-1-oxo-1,2-dihydroisoquinolin-7-yl)oxy)propanoyl)piperidine-3-carboxylic acid (Example 59, 23 mg). LCMS (ESI) Calcd. for C₂₄H₂₂ClFN₂O₅: 472, found [M+H]⁺=473. ¹H NMR (400 MHz, DMSO-d₆) δ 11.46-11.39 (br s, 1H), 7.61-7.58 (m, 2H), 7.50-7.46 (m, 1H), 7.36-7.31 (m, 1H), 7.25-7.23 (m, 1H), 6.99 (s, 1H), 6.94-6.92 (d, 1H), 5.47-5.39 (m, 1H), 4.38 (m, 1H), 4.07-3.97 (m, 1H), 3.31-3.10 (m, 1H), 2.76-2.66 (m, 2H), 2.32-2.03 (m, 1H), 2.19-1.93 (m, 2H), 1.75-1.57 (m, 2H), 1.46-1.44 (d, 3H).

### Example 60: Synthesis of (5)-1-(2-((4-(2-chloro-4-fluorophenyl)-1-oxo-1,2-dihydroisoquinolin-7-yl)oxy)propanoyl)piperidine-4-carboxylic acid

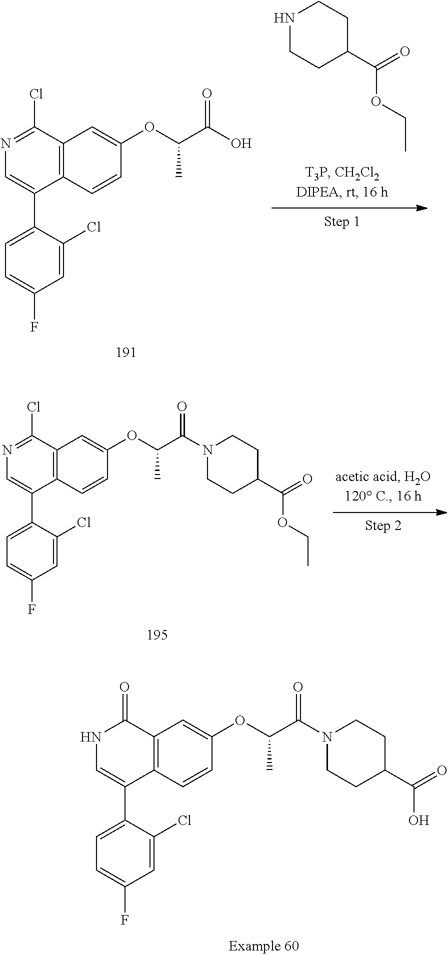 [SMILES: C[C@H](Oc1ccc2c(-c3ccc(F)cc3Cl)cnc(Cl)c2c1)C(=O)O ; CCOC(=O)C1CCNCC1 ; CCOC(=O)C1CCN(C(=O)[C@H](C)Oc2ccc3c(-c4ccc(F)cc4Cl)cnc(Cl)c3c2)CC1 ; C[C@H](Oc1ccc2c(-c3ccc(F)cc3Cl)c[nH]c(=O)c2c1)C(=O)N1CCC(C(=O)O)CC1]

Synthesis of ethyl (S)-1-(2-((1-chloro-4-(2-chloro-4-fluorophenyl)isoquinolin-7-yl)oxy)propanoyl)piperidine-4-carboxylate, 195 [Step 1]: To a stirred solution of (S)-2-((1-chloro-4-(2-chloro-4-fluorophenyl) isoquinolin-7-yl)oxy)propanoic acid (191, 95 mg, 0.3 mmol) in CH₂Cl₂ (3 mL) was added ethyl piperidine-4-carboxylate (118 mg, 0.8 mmol) followed by DIPEA (0.2 mL, 1.3 mmol) and T₃P (0.2 mL, 0.3 mmol). The reaction mixture was stirred at ambient temperature for 16 h. The reaction mixture was quenched with ice cold water and extracted with CH₂Cl₂. The organic phase was collected, washed with brine, dried over anhydrous Na₂SO₄, filtered, and concentrated under reduced pressure. The product was purified by flash chromatography to afford ethyl (S)-1-(2-((1-chloro-4-(2-chloro-4-fluorophenyl)isoquinolin-7-yl)oxy)propanoyl)piperidine-4-carboxylate (195, 110 mg). LCMS (ESI) Calcd. for C₂₆H₂₅C₁₂FN₂O₄: 518, found [M+H]⁺=519.

Synthesis of (S)-1-(2-((4-(2-chloro-4-fluorophenyl)-1-oxo-1,2-dihydroisoquinolin-7-yl)oxy)propanoyl)piperidine-4-carboxylic acid, Example 60 [Step 2]: To a stirred solution of ethyl (S)-1-(2-((1-chloro-4-(2-chloro-4-fluorophenyl)isoquinolin-7-yl)oxy)propanoyl)piperidine-4-carboxylate (195, 120 mg, 0.2 mmol) in acetic acid (1.5 mL, 27 mmol) was added water (0.4 mL, 23 mmol). The reaction mixture was stirred at 120° C. for 16 h. The reaction mixture was concentrated under reduced pressure, diluted with water, and extracted with EtOAc. The combined organic layers were washed with brine, dried over anhydrous Na₂SO₄, filtered, and concentrated under reduced pressure. The product was purified by reverse phase prep-HPLC to afford (S)-1-(2-((4-(2-chloro-4-fluorophenyl)-1-oxo-1,2-dihydroisoquinolin-7-yl)oxy)propanoyl)piperidine-4-carboxylic acid (Example 60, 15 mg). LCMS (ESI) Calcd. for C₂₄H₂₂ClFN₂O₅: 472, found [M+H]⁺=473. ¹H NMR (400 MHz, DMSO-d₆) δ 11.37 (br s, 1H), 7.61-7.59 (m, 2H), 7.49-7.46 (m, 1H), 7.35-7.31 (m, 1H), 7.25-7.23 (d, 1H), 6.98 (s, 1H), 6.94-6.92 (d, 1H), 5.43-5.41 (m, 1H), 4.23-4.20 (m, 1H), 4.04-3.96 (m, 2H), 3.14 (m, 1H), 2.95-2.90 (m, 1H), 2.74-2.66 (m, 1H), 1.90-1.71 (m, 2H), 1.44-1.39 (m, 5H).

### Example 61: Synthesis of 4-(o-tolyl)-7-((1,1,1-trifluoropropan-2-yl)oxy)isoquinolin-1(2H)-one

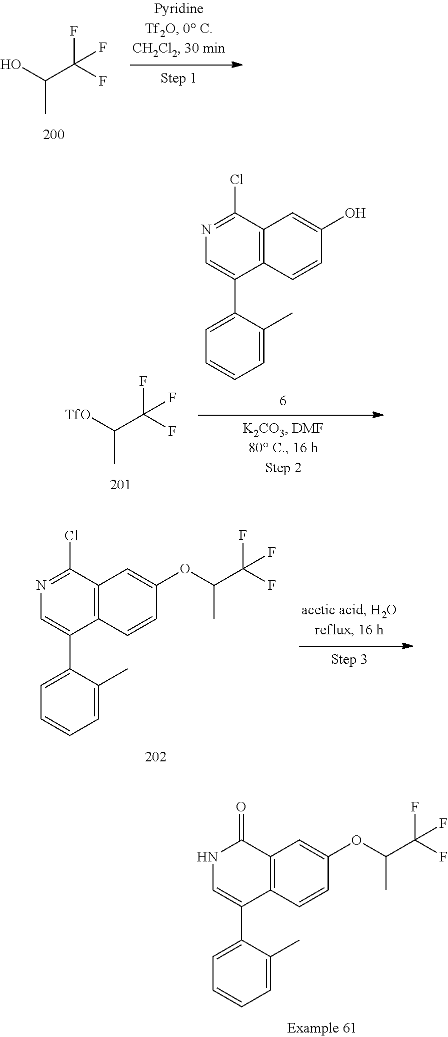

Synthesis of 1,1,1-trifluoropropan-2-yl trifluoromethanesulfonate, 201 [Step 1]: To a stirred solution of 1,1,1-trifluoropropan-2-ol (200, 400 mg, 3.5 mmol) in CH₂Cl₂ (3 mL) was added pyridine (0.4 mL, 4.2 mmol) and trifluromethanesulfonic anhydride (0.6 mL, 3.5 mmol) at 0° C. The reaction mixture was stirred at 0° C. for 15 min. The reaction mixture was filtered through a sintered funnel and washed with CH₂Cl₂. The organic layer was concentrated to half of the volume to afford 1,1,1-trifluoropropan-2-yl trifluoromethanesulfonate (201, 860 mg) as a solution in CH₂Cl₂. The product was used in the next step without further purification.

Synthesis of 1-chloro-4-(o-tolyl)-7-((1,1,1-trifluoropropan-2-yl)oxy)isoquinoline, 202 [Step 2]: To a stirred solution of 1-chloro-4-(o-tolyl)isoquinolin-7-ol (6, 150 mg, 0.6 mmol) in DMF (3 mL) was added K₂CO₃ (192 mg, 1.4 mmol) followed by a solution of 1,1,1-trifluoropropan-2-yl trifluoromethanesulfonate (201, 860 mg, 3.5 mmol in CH₂Cl₂ (2.5 mL)). The reaction mixture was stirred at 80° C. for 16 h. The reaction mixture was then quenched with ice cold water and extracted with EtOAc. The combined organic layers were washed with brine, dried over anhydrous Na₂SO₄, filtered, and concentrated under reduced pressure. The product was purified by flash chromatography to afford 1-chloro-4-(o-tolyl)-7-((1,1,1-trifluoropropan-2-yl)oxy)isoquinoline (202, 160 mg). LCMS (ESI) Calcd. for C₁₉H₁₅ClF₃NO: 365, found [M+H]⁺=366. ¹H NMR (400 MHz, DMSO-d₆) δ 8.09 (s, 1H), 7.85 (s, 1H), 7.63-7.60 (m, 1H), 7.43-7.36 (m, 4H), 7.26-7.24 (m, 1H), 5.69-5.66 (m, 1H), 1.97 (s, 3H), 1.53-1.50 (s, 3H).

Synthesis of 4-(o-tolyl)-7-((1,1,1-trifluoropropan-2-yl)oxy)isoquinolin-1(2H)-one, Example 61 [Step 3]: To a stirred solution of 1-chloro-4-(o-tolyl)-7-((1,1,1-trifluoropropan-2-yl)oxy)isoquinoline (202, 180 mg, 0.5 mmol) in acetic acid (4.2 mL, 74 mmol) was added water (1.0 mL, 56.6 mmol), and the reaction mixture was stirred at 120° C. for 16 h. The reaction mixture was concentrated under reduced pressure. The reaction mixture was diluted with water and extracted with EtOAc. The organic part was washed with brine, dried over anhydrous Na₂SO₄, and concentrated under reduced pressure. The product was purified by reverse phase prep-HPLC and lyophilized to afford 4-(o-tolyl)-7-((1,1,1-trifluoropropan-2-yl)oxy)isoquinolin-1(2H)-one (Example 61, 130 mg). LCMS (ESI) Calcd. for C₁₉H₁₆F₃NO₂: 347, found [M+H]⁺=348. ¹H NMR (400 MHz, DMSO-d₆) δ 11.42 (br s, 1H), 7.87 (m, 1H), 7.39-7.34 (m, 3H), 7.30-7.26 (m, 1H), 7.20-7.19 (d, 1H), 6.95-6.93 (d, 2H), 5.46-5.40 (m, 1H), 2.04 (s, 3H), 1.46-1.44 (d, 3H).

### Example 62: Synthesis of (S)-1-((R)-2-((4-(2-chloro-4-fluorophenyl)-1-oxo-1,2-dihydroisoquinolin-7-yl)oxy)propanoyl)piperidine-3-carboxylic acid

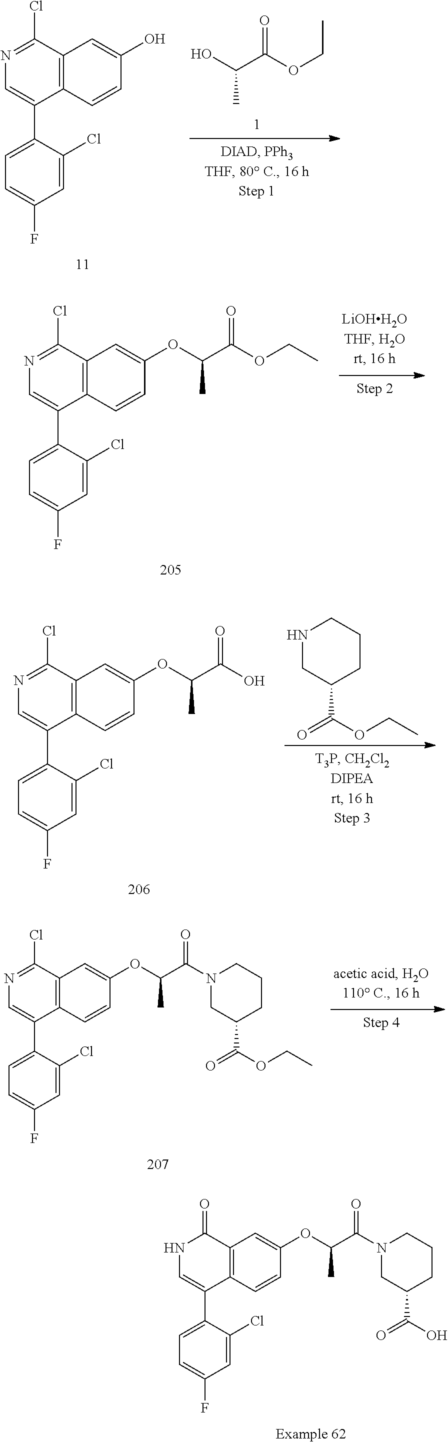

Synthesis of ethyl (R)-2-((1-chloro-4-(2-chloro-4-fluorophenyl)isoquinolin-7-yl)oxy)propanoate, 205 [Step 1]: To a stirred solution of 1-chloro-4-(2-chloro-4-fluorophenyl)isoquinolin-7-ol (11, 750 mg, 2.4 mmol) and ethyl (S)-2-hydroxypropanoate (660 mg, 5.6 mmol) in anhydrous THF (10 mL) was added PPh₃ (1.9 g, 7.3 mmol). The reaction mixture was degassed with nitrogen for 5 min. DIAD (1.4 mL, 7.3 mmol) was added, and the reaction mixture was heated at 80° C. for 16 h. The reaction mixture was diluted with EtOAc, washed with water, brine, dried over anhydrous Na₂SO₄, and concentrated under reduced pressure. The product was purified by flash column chromatography to afford ethyl (R)-2-((1-chloro-4-(2-chloro-4-fluorophenyl)isoquinolin-7-yl)oxy)propanoate (205, 900 mg). ¹H NMR (400 MHz, DMSO-d₆) δ 8.13-8.11 (m, 1H), 7.72-7.70 (m, 1H), 7.59-7.55 (m, 2H), 7.51 (br s, 1H), 7.45-7.39 (m, 2H), 5.27-5.25 (m, 1H), 4.26-4.15 (m, 2H), 1.61-1.59 (d, 3H), 1.20 (m, 3H). Synthesis of (R)-2-((1-chloro-4-(2-chloro-4-fluorophenyl)isoquinolin-7-yl)oxy)propanoic acid, 206 [Step 2]: To a stirred solution of ethyl (R)-2-((1-chloro-4-(2-chloro-4-fluorophenyl)isoquinolin-7-yl)oxy)propanoate (205, 820 mg, 2 mmol) in THF (8 mL) and water (2 mL) was added portion wise LiOH·H₂O (255 mg, 6.0 mmol). The reaction mixture was stirred at ambient temperature for 16 h. The reaction mixture was concentrated under reduced pressure, dissolved in water, and washed with ether. The aqueous layer was acidified with 1N HCl to pH=3 and extracted with EtOAc (×3). The combined organic layers were washed with brine, dried over anhydrous Na₂SO₄, filtered, and concentrated under reduced pressure to afford (R)-2-((1-chloro-4-(2-chloro-4-fluorophenyl)isoquinolin-7-yl)oxy)propanoic acid (206, 750 mg). LCMS (ESI) Calcd. for C₁₈H₁₂C₁₂FNO₃: 379, found [M+H]⁺=380. ¹H NMR (400 MHz, DMSO-d₆) δ 13.49 (br s, 1H), 8.08 (s, 1H), 7.70 (d, 1H), 7.58-7.51 (m, 3H), 7.43 (t, 1H), 7.38-7.35 (m, 1H), 5.01 (q, 1H), 1.56 (d, 3H).

Synthesis of ethyl (S)-1-((R)-2-((1-chloro-4-(2-chloro-4-fluorophenyl)isoquinolin-7-yl)oxy)propanoyl)piperidine-3-carboxylate, 207 [Step 3]: To a stirred solution of (R)-2-((1-chloro-4-(2-chloro-4-fluorophenyl)isoquinolin-7-yl)oxy)propanoic acid (207, 350 mg, 0.9 mmol) in CH₂Cl₂ (10 mL) was added ethyl (S)-piperidine-3-carboxylate (3, 215 mg, 1.4 mmol) followed by DIPEA (0.8 mL, 4.6 mmol). T₃P (0.5 mL, 1.8 mmol, 50% in EtOAc) was added to the reaction mixture at 0° C. The reaction mixture was stirred at ambient temperature for 16 h. The reaction mixture was quenched with water and extracted with CH₂C₂ (×3). The combined organic layers were washed with brine, dried over anhydrous Na₂SO₄, filtered, and concentrated under reduced pressure. The product was purified by flash column to afford ethyl ((R)-1-((R)-2-((1-chloro-4-(2-chloro-4-fluorophenyl)isoquinolin-7-yl)oxy)propanoyl)piperidine-3-carboxylate (207, 450 mg). LCMS (ESI) Calcd. for C₂₆H₂₅Cl₂FN₂O₄: 518, found [M+H]+=519.

Synthesis of (S)-1-((R)-2-((4-(2-chloro-4-fluorophenyl)-1-oxo-1,2-dihydroisoquinolin-7-yl)oxy)propanoyl)piperidine-3-carboxylic acid, Example 62 [Step 4]: To a stirred solution of ethyl (S)-1-((R)-2-((1-chloro-4-(2-chloro-4-fluorophenyl)isoquinolin-7-yl)oxy)propanoyl)piperidine-3-carboxylate (207, 240 mg, 0.5 mmol) in acetic acid (4.0 mL, 69.3 mmol) was added H₂O (0.8 mL, 46.2 mmol). The reaction mixture was heated to reflux for 16 h. The reaction mixture was concentrated under reduced pressure and diluted with EtOAc. The organic layer was washed with water, brine, dried over anhydrous Na₂SO₄, filtered, and concentrated under reduced pressure. The product was purified by reverse phase prep-HPLC and lyophilized to afford (S)-1-((R)-2-((4-(2-chloro-4-fluorophenyl)-1-oxo-1,2-dihydroisoquinolin-7-yl)oxy)propanoyl)piperidine-3-carboxylic acid (Example 62, 60 mg). LCMS (ESI) Calcd. for C₂₄H₂₂ClFN₂O₅: 472, found [M+H]⁺=473. ¹H NMR (400 MHz, DMSO-d₆) δ 11.40 (br s, 1H), 7.61-7.55 (m, 2H), 7.47-7.46 (m, 1H), 7.36-7.31 (m, 1H), 7.25-7.23 (m, 1H), 6.99 (s, 1H), 6.93 (d, 1H), 5.43 (d, 1H), 4.42 (d, 1H), 4.02 (br s, 2H), 3.12-3.06 (m, 1H), 2.71-2.66 (m, 1H), 2.32 (br s, 1H), 2.08-2.05 (m, 1H), 1.90-1.84 (m, 1H), 1.75 (br s, 1H), 1.61-1.58 (m, 1H), 1.47-1.43 (m, 3H).

### Example 63: Synthesis of 2-((1-oxo-4-(o-tolyl)-1,2-dihydroisoquinolin-7-yl)oxy)acetamide

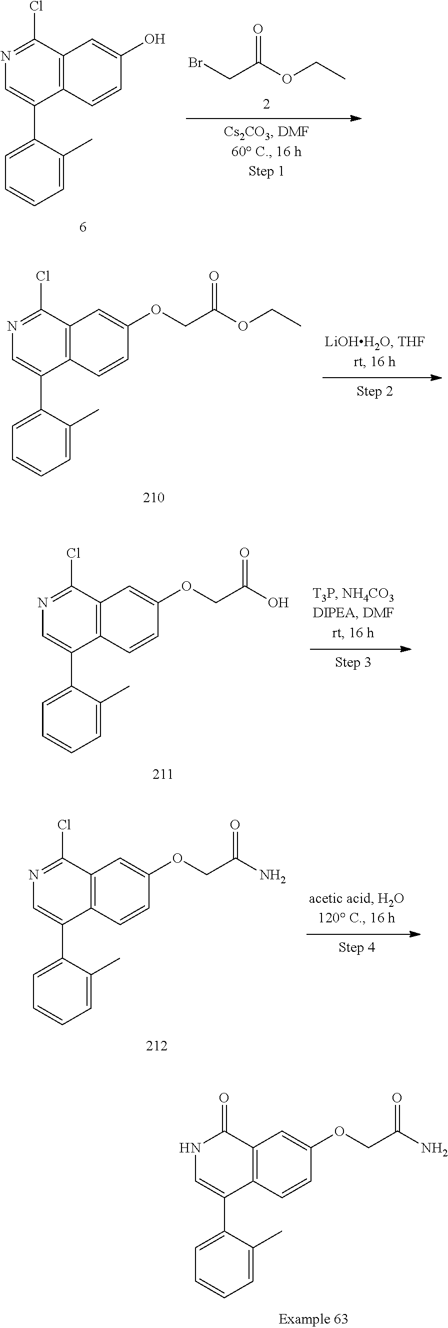

Synthesis of ethyl 2-((1-chloro-4-(o-tolyl)isoquinolin-7-yl)oxy)acetate, 210 [Step 1]: To a stirred solution of 1-chloro-4-(o-tolyl)isoquinolin-7-ol (6, 150 mg, 0.6 mmol) in DMF (3 mL) was added ethyl 2-bromoacetate (150 mg, 0.9 mmol) and Cs₂CO₃ (255 mg, 1.9 mmol). The reaction mixture was stirred for 2 h. at 80° C. The reaction mixture was quenched with ice cold water and extracted with EtOAc (×3). The combined organic layers were washed with water, brine, dried over anhydrous Na₂SO₄, filtered, and concentrated under reduced pressure. The product was purified by flash chromatography to afford ethyl 2-((1-chloro-4-(o-tolyl)isoquinolin-7-yl)oxy)acetate (210, 180 mg). LCMS (ESI) Calcd. for C₂₀H₁₈ClNO₃: 355, found [M+H]⁺=356.

Synthesis of 2-((1-chloro-4-(o-tolyl)isoquinolin-7-yl)oxy)acetic acid, 211 [Step 2]: To a stirred solution of ethyl 2-((1-chloro-4-(o-tolyl)isoquinolin-7-yl)oxy)acetate (210, 180 mg, 0.5 mmol) in THF (3 mL) was added dropwise LiOH·H₂O (65 mg, 1.5 mmol) dissolved in water (0.75 mL). The reaction mixture was stirred at ambient temperature for 2 h. The reaction mixture was concentrated under reduced pressure, diluted with water, acidified with citric acid, and extracted with EtOAc (×3). The combined organic layers were washed with brine, dried over anhydrous Na₂SO₄, filtered, and concentrated under reduced pressure to afford 2-((1-chloro-4-(o-tolyl)isoquinolin-7-yl)oxy)acetic acid (211, 150 mg). LCMS (ESI) Calcd. for C₁₈H₁₄ClNO₃: 327, found [M+H]⁺=328.

Synthesis of 2-((1-chloro-4-(o-tolyl)isoquinolin-7-yl)oxy)acetamide, 212 [Step 3]: To a stirred solution of 2-((1-chloro-4-(o-tolyl)isoquinolin-7-yl)oxy)acetic acid (211, 170 mg, 0.5 mmol) in DMF (3 mL) was added cold DIPEA (0.4 mL, 2.6 mmol), (NH₄)₂CO₃ (250 mg, 2.6 mmol), and T₃P (0.5 mL, 0.8 mmol, 50% in EtOAc). The reaction mixture was stirred at ambient temperature for 16 h. The reaction mixture was quenched with ice cold water and extracted with EtOAc. The combined organic layers were washed with brine, dried over anhydrous Na₂SO₄, filtered, and concentrated under reduced pressure. The product was purified by column chromatography over silica gel to afford 2-((1-chloro-4-(o-tolyl)isoquinolin-7-yl)oxy)acetamide (212, 160 mg). LCMS (ESI) Calcd. for C₁₈H₁₅ClN₂O₂: 326, found [M+H]⁺=327.

Synthesis of 2-((1-oxo-4-(o-tolyl)-1,2-dihydroisoquinolin-7-yl)oxy)acetamide, Example 63 [Step 4]: To a stirred solution of 2-((1-chloro-4-(o-tolyl)isoquinolin-7-yl)oxy)acetamide (212, 150 mg, 0.5 mmol) in acetic acid (4.0 mL, 70 mmol) was added water (0.8 mL, 46 mmol). The reaction mixture was stirred at 120° C. for 16 h. The reaction mixture was concentrated under reduced pressure, diluted with water, and extracted with EtOAc (×3). The combined organic layers were washed with brine, dried over anhydrous Na₂SO₄, filtered, and concentrated under reduced pressure. The product was purified by reverse phase prep-HPLC and lyophilized to afford 2-((1-oxo-4-(o-tolyl)-1,2-dihydroisoquinolin-7-yl)oxy)acetamide (Example 63, 10 mg). LCMS (ESI) Calcd. for C₁₈H₁₆N₂O₃: 308, found [M+H]⁺=309. ¹H NMR (400 MHz, DMSO-d₆) δ 11.36 (d, 1H), 7.67 (d, 1H), 7.65-7.61 (m, 1H), 7.39 (s, 1H), 7.35-7.30 (m, 4H), 7.20 (d, 1H), 6.93-6.88 (dd, 2H), 4.55 (s, 2H), 2.03 (s, 3H).

### Example 64: Synthesis of (R)-1-(2-((4-(2-chloro-4-fluorophenyl)-1-oxo-1,2-dihydroisoquinolin-7-yl)oxy)propanoyl)piperidine-4-carboxylic acid

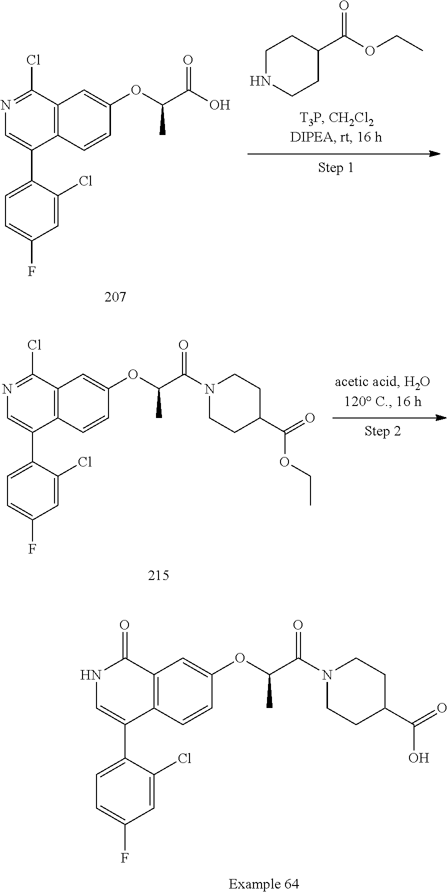 [SMILES: C[C@@H](Oc1ccc2c(-c3ccc(F)cc3Cl)cnc(Cl)c2c1)C(=O)O ; CCOC(=O)C1CCNCC1 ; CCOC(=O)C1CCN(C(=O)[C@@H](C)Oc2ccc3c(-c4ccc(F)cc4Cl)cnc(Cl)c3c2)CC1 ; C[C@@H](Oc1ccc2c(-c3ccc(F)cc3Cl)c[nH]c(=O)c2c1)C(=O)N1CCC(C(=O)O)CC1]

Synthesis of ethyl (R)-1-(2-((1-chloro-4-(2-chloro-4-fluorophenyl)isoquinolin-7-yl)oxy)propanoyl)piperidine-4-carboxylate, 215 [Step 1]: To a stirred solution of (R)-2-((1-chloro-4-(2-chloro-4-fluorophenyl)isoquinolin-7-yl)oxy)propanoic acid (207, 100 mg, 0.3 mmol) in CH₂Cl₂ (3 mL) was added ethyl piperidine-4-carboxylate (90 mg, 0.5 mmol) followed by DIPEA (0.14 mL, 1 mmol). T₃P (0.1 mL, 0.4 mmol, 50% in EtOAc) was added to the reaction mixture at 0° C. The reaction mixture was stirred at ambient temperature for 16 h. The reaction mixture was concentrated under reduced pressure, diluted in water, and extracted in EtOAc. The combined organic layers were washed with brine, dried over anhydrous Na₂SO₄, filtered, and concentrated under reduced pressure. The product was purified by flash chromatography to afford ethyl (R)-1-(2-((1-chloro-4-(2-chloro-4-fluorophenyl)isoquinolin-7-yl)oxy)propanoyl)piperidine-4-carboxylate (215, 100 mg). LCMS (ESI) Calcd. for C₂₆H₂₅Cl₂FN₂O₄: 518, found [M+H]⁺=519.

Synthesis of (R)-1-(2-((4-(2-chloro-4-fluorophenyl)-1-oxo-1,2-dihydroisoquinolin-7-yl)oxy)propanoyl)piperidine-4-carboxylic acid, Example 64 [Step 2]: To a stirred solution of ethyl (R)-1-(2-((1-chloro-4-(2-chloro-4-fluorophenyl)isoquinolin-7-yl)oxy)propanoyl)piperidine-4-carboxylate (215, 110 mg, 0.2 mmol) in acetic acid (4.2 mL, 74.1 mmol) was added water (0.9 mL, 52.9 mmol). The reaction mixture was heated to reflux for 16 h. The reaction mixture was concentrated under reduced pressure and dissolved in EtOAc. The organic phase was washed with water, brine, dried over anhydrous Na₂SO₄, filtered, and concentrated under reduced pressure. The product was purified by reverse phase prep-HPLC and lyophilized to afford (R)-1-(2-((4-(2-chloro-4-fluorophenyl)-1-oxo-1,2-dihydroisoquinolin-7-yl)oxy)propanoyl)piperidine-4-carboxylic acid (Example 64, 28 mg). LCMS (ESI) Calcd. for C₂₄H₂₂ClFN₂O₅: 472, found [M+H]⁺=473. ¹H NMR (400 MHz, DMSO-d₆) δ 12.25 (br s, 1H), 11.41 (m, 1H), 7.61-7.58 (m, 2H), 7.49-7.46 (m, 1H), 7.36-7.31 (m, 1H), 7.25-7.23 (d, 1H), 6.99 (s, 1H), 6.94-6.92 (d, 1H), 5.43-5.41 (m, 1H), 4.20-4.01 (m, 3H), 3.49-3.40 (m, 1H), 3.17 (m, 1H), 1.90-1.75 (m, 3H), 1.46-1.44 (m, 4H).

### Example 65: Synthesis of 4-(o-tolyl)-7-(2,2,2-trifluoroethoxy)isoquinolin-1(2H)-one

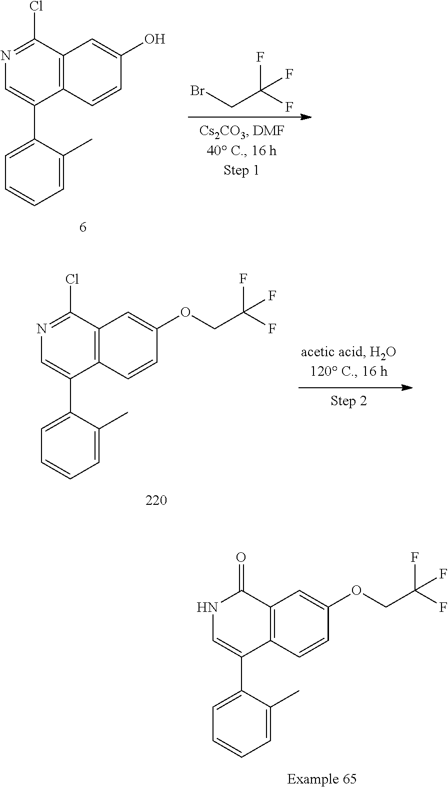 [SMILES: Cc1ccccc1-c1cnc(Cl)c2cc(O)ccc12 ; FC(F)(F)CBr ; Cc1ccccc1-c1cnc(Cl)c2cc(OCC(F)(F)F)ccc12 ; Cc1ccccc1-c1c[nH]c(=O)c2cc(OCC(F)(F)F)ccc12]

Synthesis of 1-chloro-4-(o-tolyl)-7-(2,2,2-trifluoroethoxy)isoquinoline, 220 [Step 1]: To a stirred solution of 1-chloro-4-(o-tolyl)isoquinolin-7-ol (6, 200 mg, 0.7 mmol) in DMF (2 mL) was added Cs₂CO₃ (725 mg, 2.2 mmol) and 2-bromo-1,1,1-trifluoro-ethane (240 mg, 1.5 mmol). The reaction mixture was allowed to stir at 40° C. for 16 h. The reaction mixture was diluted with EtOAc, washed with water, brine, dried over anhydrous Na₂SO₄, filtered, and concentrated under reduced pressure to afford 1-chloro-4-(o-tolyl)-7-(2,2,2-trifluoroethoxy)isoquinoline (220, 200 mg). LCMS (ESI) Calcd. for C₂₀H₁₈ClNO₃: 351, found [M+H]⁺=352.

Synthesis of 4-(o-tolyl)-7-(2,2,2-trifluoroethoxy)isoquinolin-1(2H)-one, Example 65 [Step 2]: To a stirred solution of 1-chloro-4-(o-tolyl)-7-(2,2,2-trifluoroethoxy)isoquinoline (220, 115 mg, 0.3 mmol) in acetic acid (6.7 mL, 117 mmol) was added water (1.5 mL, 83.3 mmol). The reaction mixture was heated to reflux for 16 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford 4-(o-tolyl)-7-(2,2,2-trifluoroethoxy)isoquinolin-1(2H)-one (Example 65, 67 mg). LCMS (ESI) Calcd. for C₁₈H₁₄F₃NO₂: 333, found [M+H]⁺=334. ¹H NMR (400 MHz, DMSO-d₆) δ 11.42 (d, 1H), 7.83 (d, 1H), 7.39-7.34 (m, 3H), 7.30-7.26 (m, 1H), 7.21-7.19 (d, 1H), 6.95-6.93 (m, 2H), 4.95 (m, 2H), 2.03 (s, 3H).

### Example 66: Synthesis 2-(methyl(1-oxo-4-(o-tolyl)-1,2-dihydroisoquinolin-7-yl)amino)acetamide

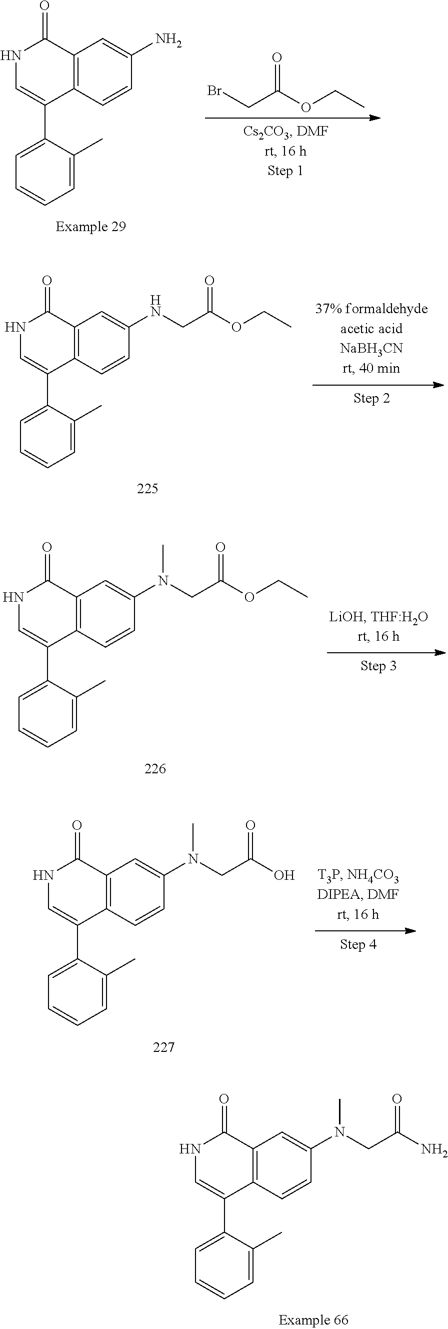

Synthesis of ethyl (1-oxo-4-(o-tolyl)-1,2-dihydroisoquinolin-7-yl)glycinate, 225 [Step 1]: To a stirred solution of 7-amino-4-(o-tolyl)-(2H)isoquinolin-1-one (Example 29, 300 mg, 1.2 mmol) in DMF (3 mL) was added DIPEA (0.5 mL, 3.6 mmol), ethyl 2-bromoacetate (0.27 mL, 2.4 mmol), and NaI (40 mg, 0.2 mmol). The reaction mixture was stirred at ambient temperature for 12 h. The reaction mixture was diluted with EtOAc, washed with water, brine, dried over anhydrous Na₂SO₄, filtered, and concentrated under reduced pressure. The product was purified by flash column chromatography to afford ethyl 2-[[4-(o-tolyl)-1-oxo-(2H)isoquinolin-7-yl]amino]acetate (225, 220 mg). LCMS (ESI) Calcd. for C₂₀H₂₀N₂O₃: 336, found [M+H]⁺=337.

Synthesis of ethyl N-methyl-N-(1-oxo-4-(o-tolyl)-1,2-dihydroisoquinolin-7-yl)glycinate, 226 [Step 2]: To a stirred solution of ethyl 2-[[4-(o-tolyl)-1-oxo-(2H)isoquinolin-7-yl]amino]acetate (225, 215 mg, 0.6 mmol) in acetic acid (1 mL) was added formaldehyde (0.48 mL, 6.3 mmol, 37% in aq. solution). The reaction mixture was stirred at ambient temperature for 5 min. NaBH₃CN (120 mg, 1.9 mmol) was added, and the reaction mixture was stirred for 40 min. The reaction mixture was quenched with water and extracted with EtOAc. The organic layer was washed with water, brine, dried over anhydrous Na₂SO₄, filtered, and concentrated under reduced pressure. The product was purified by flash chromatography to afford ethyl N-methyl-N-(1-oxo-4-(o-tolyl)-1,2-dihydroisoquinolin-7-yl)glycinate (226, 127 mg). LCMS (ESI) Calcd. for C₂₁H₂₂N₂O₃: 350, found [M+H]⁺=351. ¹H NMR (400 MHz, DMSO-d₆) δ 11.15 (d, 1H), 7.38-7.32 (m, 4H), 7.18 (m, 1H), 7.15-7.11 (m, 1H), 6.82-6.80 (d, 1H), 6.73-6.72 (d, 1H), 4.29 (s, 2H), 4.10-4.08 (m, 2H), 3.06 (s, 3H), 2.04 (s, 3H), 1.18-1.15 (m, 3H).

Synthesis of N-methyl-N-(1-oxo-4-(o-tolyl)-1,2-dihydroisoquinolin-7-yl)glycine, 227 [Step 3]: To a stirred solution of ethyl N-methyl-N-(1-oxo-4-(o-tolyl)-1,2-dihydroisoquinolin-7-yl)glycinate (226, 127 mg, 0.4 mmol) in THF (4 mL) and water (1 mL) was added LiOH·H₂O (61 mg, 1.4 mmol). The reaction mixture was stirred at ambient temperature for 16 h. The reaction mixture was concentrated under reduced pressure. The reaction mixture was diluted with water, acidified with citric acid, and extracted with EtOAc. The combined organic layers were washed with water, brine, dried over anhydrous Na₂SO₄, filtered, and concentrated under reduced pressure to afford N-methyl-N-(1-oxo-4-(o-tolyl)-1,2-dihydroisoquinolin-7-yl)glycine (227, 110 mg). LCMS (ESI) Calcd. for C₁₉H₁₈N₂O₃: 322, found [M+H]⁺=323.

Synthesis 2-(methyl(1-oxo-4-(o-tolyl)-1,2-dihydroisoquinolin-7-yl)amino)acetamide, Example 66 [Step 4]: To a stirred solution of N-methyl-N-(1-oxo-4-(o-tolyl)-1,2-dihydroisoquinolin-7-yl)glycine (227, 115 mg, 0.4 mmol) in DMF (3 mL) was added (NH₄)₂CO₃ (343 mg, 3.6 mmol) followed by DIPEA (0.31 mL, 1.8 mmol) and T₃P (0.32 mL, 0.5 mmol) at 0° C. The reaction mixture was stirred at ambient temperature for 16 h. The reaction mixture was diluted with ice cold water and extracted with EtOAc. The combined organic layers were washed with brine, dried over anhydrous Na₂SO₄, filtered, and concentrated under reduced pressure. The product was purified by reverse phase prep-HPLC and lyophilized to afford 2-(methyl(1-oxo-4-(o-tolyl)-1,2-dihydroisoquinolin-7-yl)amino)acetamide (Example 66, 70 mg). LCMS (ESI) Calcd. for C₂₀H₁₉NO₅: 321, found [M+H]⁺=322. ¹H NMR (400 MHz, DMSO-d₆) δ 11.11 (d, 1H), 7.38-7.32 (m, 4H), 7.28-7.24 (m, 1H), 7.17-7.15 (d, 1H), 7.07-7.04 (m, 2H), 6.82-6.80 (d, 1H), 6.70-6.79 (d, 1H), 3.95 (s, 2H), 3.06 (s, 3H), 2.04 (s, 3H).

### Example 67-69: Synthesis of 2-((1-oxo-4-(o-tolyl)-1,2-dihydroisoquinolin-7-yl)amino)propenamide and chiral 2-((1-oxo-4-(o-tolyl)-1,2-dihydroisoquinolin-7-yl)amino)propenamide

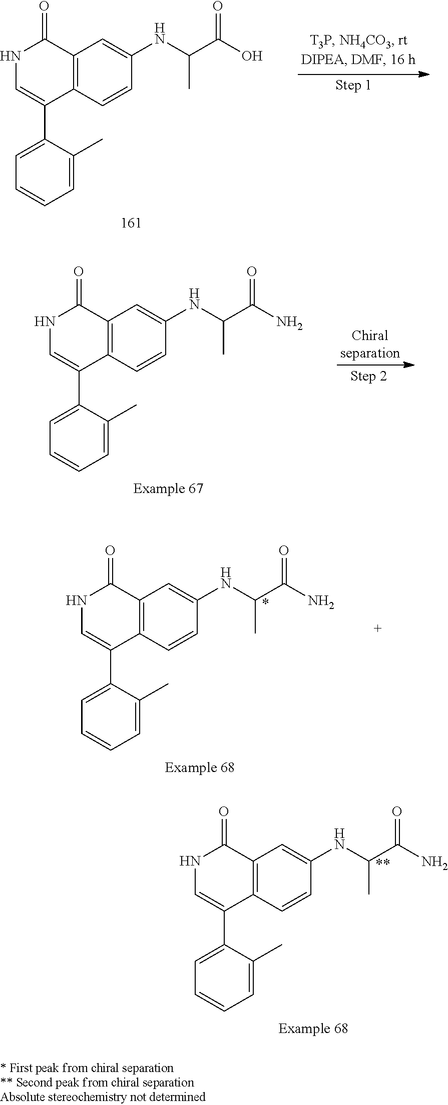 [SMILES: Cc1ccccc1-c1c[nH]c(=O)c2cc(NC(C)C(=O)O)ccc12 ; Cc1ccccc1-c1c[nH]c(=O)c2cc(NC(C)C(N)=O)ccc12 ; Cc1ccccc1-c1c[nH]c(=O)c2cc(NC(C)C(N)=O)ccc12 ; Cc1ccccc1-c1c[nH]c(=O)c2cc(NC(C)C(N)=O)ccc12]

Synthesis of 2-((1-oxo-4-(o-tolyl)-1,2-dihydroisoquinolin-7-yl)amino)propenamide, Example 67 [Step 1]: To a stirred solution of (1-oxo-4-(o-tolyl)-1,2-dihydroisoquinolin-7-yl)alanine (161, 289 mg, 0.9 mmol) in DMF (3 mL) was added (NH₄)₂CO₃ (861 mg, 8.9 mmol), DIPEA (0.78 mL, 4.5 mmol), and T₃P (0.79 mL, 1.3 mmol, 50% in EtOAc). The reaction mixture was stirred at ambient temperature for 16 h. The reaction mixture was diluted with ice cold water and extracted with EtOAc. The organic layer was washed with brine, dried over anhydrous Na₂SO₄, filtered, and concentrated under reduced pressure. The product was purified by reverse phase prep-HPLC and lyophilized to afford 2-((1-oxo-4-(o-tolyl)-1,2-dihydroisoquinolin-7-yl)amino)propanamide (Example 67, 80 mg). LCMS (ESI) Calcd. for C₁₉H₁₉N₃O₂: 321, found [M+H]⁺=322. ¹H NMR (400 MHz, DMSO-d₆) δ 11.07 (m, 1H), 7.43 (d, 1H), 7.32 (m, 2H), 7.25 (m, 2H), 7.16 (d, 1H), 7.01 (m, 2H), 6.73 (d, 1H), 6.67 (d, 1H), 6.22 (d, 1H), 3.86-3.83 (m, 1H), 2.03 (s, 3H), 1.34 (d, 3H).

Synthesis of chiral 2-((1-oxo-4-(o-tolyl)-1,2-dihydroisoquinolin-7-yl)amino)propenamide, Examples 68 and 69 [Step 2]: 2-((1-oxo-4-(o-tolyl)-1,2-dihydroisoquinolin-7-yl)amino)propanamide (Example 67, 60 mg) was purified by chiral separation by the SFC method and lyophilized. The first product was isolated as 2-((1-oxo-4-(o-tolyl)-1,2-dihydroisoquinolin-7-yl)amino)propenamide, Peak 1 (Example 68, 20 mg) and the second product as 2-((1-oxo-4-(o-tolyl)-1,2-dihydroisoquinolin-7-yl)amino)propenamide, Peak 2 (Example 69, 18 mg). The absolute stereochemistry for these Examples was not determined.

Example 68: 2-((1-oxo-4-(o-tolyl)-1,2-dihydroisoquinolin-7-yl)amino)propenamide, Peak 1: LCMS (ESI) Calcd. for C₁₉H₁₉N₃O₂: 321, found [M+H]⁺=322. ¹H NMR (400 MHz, DMSO-d₆) δ 11.06 (s, 1H), 7.42 (m, 1H), 7.31 (m, 2H), 7.26 (m, 2H), 7.16 (m, 1H), 7.00 (m, 2H), 6.73 (m, 1H), 6.67 (br s, 1H), 6.21 (m, 1H), 3.85 (m, 1H), 2.03 (s, 3H), 1.34 (m, 3H).

Example 69: 2-((1-oxo-4-(o-tolyl)-1,2-dihydroisoquinolin-7-yl)amino)propenamide, Peak 2: LCMS (ESI) Calcd. for C₁₉H₁₉N₃O₂: 321, found [M+H]⁺=322. ¹H NMR (400 MHz, DMSO-d₆) δ 11.06 (s, 1H), 7.42 (m, 1H), 7.32 (m, 2H), 7.25 (m, 2H), 7.16 (m, 1H), 7.00 (m, 2H), 6.73 (m, 1H), 6.67 (br s, 1H), 6.21 (m, 1H), 3.85 (m, 1H), 2.03 (s, 3H), 1.34 (m, 3H).

Chiral SFC: Chiral separation was performed on a Thar SFC-80 series instrument. Column was a Regis Reflect (R,R) whelk-01 (21.1×250 mm), 5 μm, operating at 35° C. with flow rate of 60 gm/min. Mobile phase: 75% super critical CO₂ and 25% of 0.3% isopropylamine in MeOH, held isocratic for up to 12 min. at 100 bar with detection at 230 nm wavelength.

### Example 70: Synthesis of 2-((1-oxo-4-(o-tolyl)-1,2-dihydroisoquinolin-7-yl)amino)acetamide

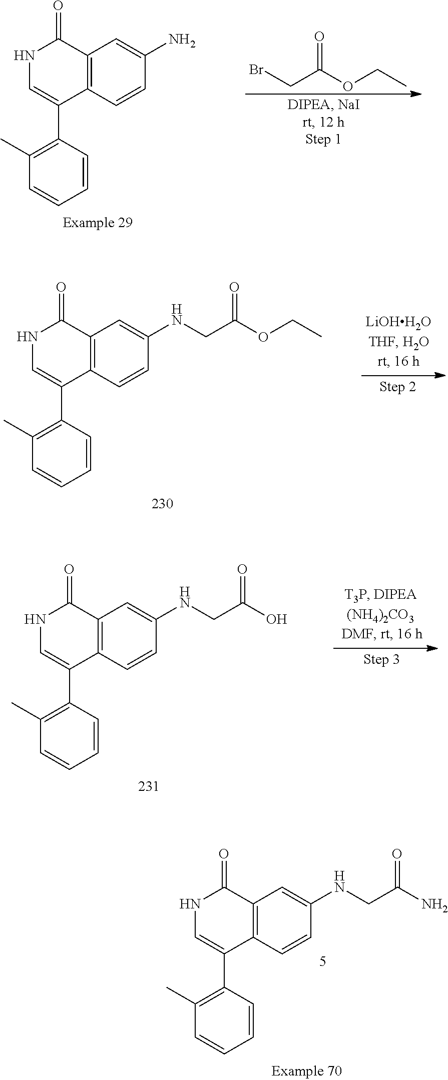

Synthesis of ethyl (1-oxo-4-(o-tolyl)-1,2-dihydroisoquinolin-7-yl)glycinate, 230 [Step 1]: To stirred a solution of ethyl 2-bromoacetate (0.2 mL, 1.8 mmol) in DMF (2 mL) was added DIPEA (0.24 mL, 1.8 mmol). The reaction mixture was stirred at ambient temperature for 10 min. prior to addition of 7-Amino-4-(o-tolyl)isoquinolin-1(2H)-one (Example 29, 150 mg, 0.6 mmol) and NaI (20 mg, 0.1 mmol). The reaction mixture was stirred at ambient temperature for 12 h. The reaction mixture was diluted with water and extracted with EtOAc. The combined organic layers were washed with brine, dried over anhydrous NaSO₄, filtered, and concentrated under reduced pressure. The product was purified by flash column chromatography to afford ethyl (1-oxo-4-(o-tolyl)-1,2-dihydroisoquinolin-7-yl)glycinate (230, 133 mg). LCMS (ESI) Calcd. for C₂₀H₂₀N₂O₃: 336, found [M+H]⁺=337.

Synthesis of (1-oxo-4-(o-tolyl)-1,2-dihydroisoquinolin-7-yl)glycine, 231 [Step 2]: To a stirred solution of ethyl (1-oxo-4-(o-tolyl)-1,2-dihydroisoquinolin-7-yl)glycinate (230, 133 mg, 0.4 mmol) in THF (4 mL) and water (1 mL) was added LiOH·H₂O (66 mg, 1.6 mmol). The reaction mixture was stirred at ambient temperature for 16 h. The reaction mixture was concentrated under reduced pressure. The reaction mixture was diluted with water, acidified with citric acid, and extracted with EtOAc. The combined organic layers were washed with water, brine, dried over anhydrous Na₂SO₄, filtered, and concentrated under reduced pressure to afford (1-oxo-4-(o-tolyl)-1,2-dihydroisoquinolin-7-yl)glycine (231, 104 mg). The product was used in the next step without further purification. LCMS (ESI) Calcd. for C₁₈H₁₆N₂O₃: 308, found [M+H]⁺=309.

Synthesis of 2-((1-oxo-4-(o-tolyl)-1,2-dihydroisoquinolin-7-yl)amino)acetamide, Example 70 [Step 3]: To a stirred solution of (1-oxo-4-(o-tolyl)-1,2-dihydroisoquinolin-7-yl)glycine (231, 135 mg, 0.4 mmol) in DMF (3 mL) was added (NH₄)₂CO₃ (421 mg, 4.3 mmol) followed by DIPEA (0.38 mL, 2.2 mmol). T₃P (50% in EtOAc) (0.39 mL, 0.7 mmol) was added at 0° C. The reaction mixture was allowed to warm up to ambient temperature and stir for 16 h. The reaction mixture was quenched with ice cold water and extracted with EtOAc. The combined organic layers were washed with brine, dried over anhydrous Na₂SO₄, filtered, and concentrated under reduced pressure. The product was purified by reverse phase prep-HPLC and lyophilized to afford 2-((1-oxo-4-(o-tolyl)-1,2-dihydroisoquinolin-7-yl)amino)acetamide (Example 70, 50 mg). LCMS (ESI) Calcd. for C₁₈H₁₇N₃O₂: 307, found [M+H]⁺=308. ¹H NMR (400 MHz, DMSO-d₆) δ 11.08 (d, 1H), 7.41 (br s, 1H), 7.32 (m, 2H), 7.26-7.25 (m, 1H), 7.21 (m, 1H), 7.17-7.15 (d, 1H), 7.10 (br s, 1H), 7.02-7.00 (m, 1H), 6.74-6.72 (d, 1H), 6.68-6.66 (d, 1H), 6.33 (m, 1H), 3.68 (d, 2H), 2.03 (s, 3H).

### Example 71-73: Synthesis of 2-((1-oxo-4-(o-tolyl)-1,2-dihydroisoquinolin-7-yl)oxy)propenamide and chiral 2-((1-oxo-4-(o-tolyl)-1,2-dihydroisoquinolin-7-yl)oxy)propenamide

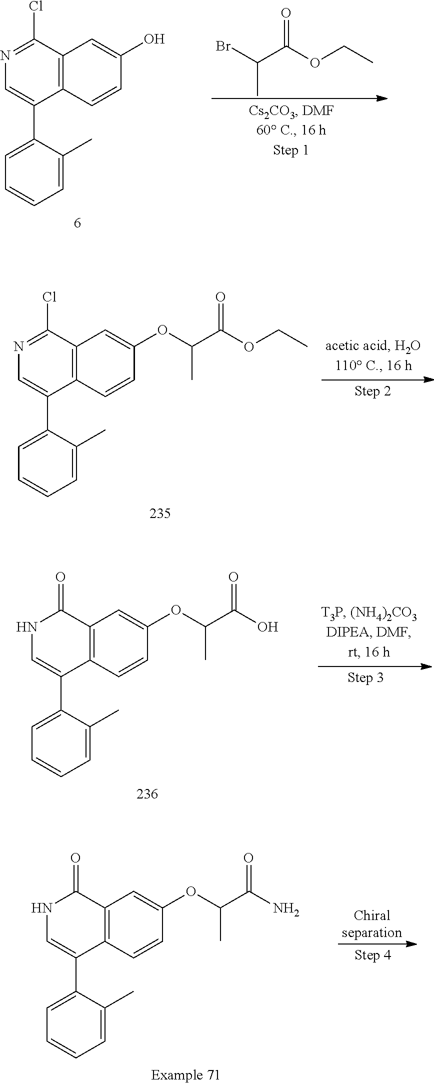

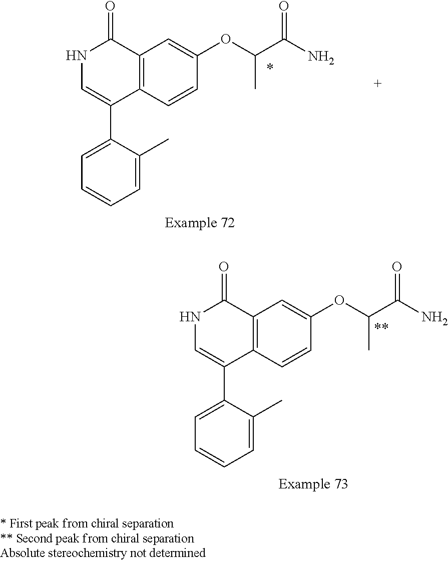 [SMILES: Cc1ccccc1-c1c[nH]c(=O)c2cc(OC(C)C(N)=O)ccc12 ; Cc1ccccc1-c1c[nH]c(=O)c2cc(OC(C)C(N)=O)ccc12]

Synthesis of ethyl 2-((1-chloro-4-(o-tolyl)isoquinolin-7-yl)oxy)propanoate, 235 [Step 1]: To a stirred solution of 1-chloro-4-(o-tolyl)isoquinolin-7-ol (6, 200 mg, 0.7 mmol) in DMF (2 mL) was added Cs₂CO₃ (725 mg, 2.2 mmol) and ethyl 2-bromopropanoate (270 mg, 1.5 mmol). The reaction mixture was stirred at 80° C. for 16 h. The reaction mixture was diluted with EtOAc, washed with water, brine, dried over anhydrous Na₂SO₄, filtered, and concentrated to afford ethyl 2-((1-chloro-4-(o-tolyl)isoquinolin-7-yl)oxy)propanoate (235, 240 mg). LCMS (ESI) Calcd. For C₂₁H₂₀ClNO₃: 369, found [M+H]⁺=370.

Synthesis of 2-((1-oxo-4-(o-tolyl)-1,2-dihydroisoquinolin-7-yl)oxy)propanoic acid, 236 [Step 2]: To a stirred solution of ethyl 2-((1-chloro-4-(o-tolyl)isoquinolin-7-yl)oxy)propanoate (235, 300 mg, 0.8 mmol) in acetic acid (16 mL, 285 mmol) was added water (2.3 mL, 130 mmol). The reaction mixture was heated to reflux for 16 h. The reaction mixture was concentrated under reduced pressure. The reaction mixture was dissolved in EtOAc, washed with water, brine, dried over anhydrous Na₂SO₄, filtered, and concentrated under reduced pressure to afford 2-((1-oxo-4-(o-tolyl)-1,2-dihydroisoquinolin-7-yl)oxy)propanoic acid (236, 260 mg). LCMS (ESI) Calcd. for C₁₉H₁₇NO₄: 323, found [M+H]⁺=324.

Synthesis of 2-((1-oxo-4-(o-tolyl)-1,2-dihydroisoquinolin-7-yl)oxy)propenamide, Example 71 [Step 3]: To a stirred solution of 2-((1-oxo-4-(o-tolyl)-1,2-dihydroisoquinolin-7-yl)oxy)propanoic acid (236, 315 mg, 10.0 mmol) in DMF (3 mL) was added (NH₄)₂CO₃ (470 mg, 4.9 mmol), DIPEA (0.85 mL, 4.9 mmol), and T₃P (0.9 mL, 1.4 mmol, 50% in EtOAc). The reaction mixture was stirred at ambient temperature for 16 h. The reaction mixture was then diluted with ice cold water and extracted with EtOAc. The combined organic layers were washed with brine, dried over anhydrous Na₂SO₄, filtered, and concentrated under reduced pressure. The product was purified by flash chromatography to afford 2-((1-oxo-4-(o-tolyl)-1,2-dihydroisoquinolin-7-yl)oxy)propanamide (Example 71, 315 mg). LCMS (ESI) Calcd. for C₁₉H₁₈N₂O₃: 322, found [M+H]⁺=323.

Synthesis of chiral 2-((1-oxo-4-(o-tolyl)-1,2-dihydroisoquinolin-7-yl)oxy)propanamide, Examples 72 and 73 [Step 4]: 2-((1-oxo-4-(o-tolyl)-1,2-dihydroisoquinolin-7-yl)oxy)propanamide (Example 71, 40 mg) was purified by normal phase chiral prep-HPLC. The first product was isolated as 2-((1-oxo-4-(o-tolyl)-1,2-dihydroisoquinolin-7-yl)oxy)propenamide, Peak 1 (Example 72, 5.0 mg) and the second as 2-((1-oxo-4-(o-tolyl)-1,2-dihydroisoquinolin-7-yl)oxy)propenamide, Peak 2 (Example 73, 5.0 mg). The absolute stereochemistry for these Examples was not determined.

Example 72: 2-((1-oxo-4-(o-tolyl)-1,2-dihydroisoquinolin-7-yl)oxy)propenamide, Peak 1: LCMS (ESI) Calcd. for C₁₉H₁₈N₂O₃: 322, found [M+H]+=323. ¹H NMR (400 MHz, DMSO-d₆) δ 11.33 (br s, 1H), 7.66 (br s, 1H), 7.61 (d, 1H), 7.34-7.18 (m, 6H), 6.92-6.87 (m, 2H), 4.74 (br s, 1H), 2.04 (s, 3H), 1.48 (d, 3H).

Example 73: 2-((1-oxo-4-(o-tolyl)-1,2-dihydroisoquinolin-7-yl)oxy)propenamide, Peak 2: LCMS (ESI) Calcd. for C₁₉H₁₈N₂O₃: 322, found [M+H]+=323. ¹H NMR (400 MHz, DMSO-d₆) δ 11.33 (br s, 1H), 7.66 (br s, 1H), 7.61 (d, 1H), 7.34-7.18 (m, 6H), 6.92-6.87 (m, 2H), 4.74 (br s, 1H), 2.04 (s, 3H), 1.48 (d, 3H).

Chiral prep-HPLC: Chiral separation was performed on an Agilent 1200 series instrument. Column was a Chiralpak IG (250×21 mm), 5 μm, operating at ambient temperature with flow rate of 21.0 mL/min. Mobile phase: 0.1% isopropylamine in a mixture of 60% hexane, 20% CH₂Cl₂, and 20% ethanol, held isocratic for up to 25 min. with detection at 228 nm wavelength.

### Examples 74-76: Synthesis of 3-methoxy-2-((1-oxo-4-(o-tolyl)-1,2-dihydroisoquinolin-7-yl)oxy)propanenitrile and chiral 3-methoxy-2-((1-oxo-4-(o-tolyl)-1,2-dihydroisoquinolin-7-yl)oxy)propanenitrile

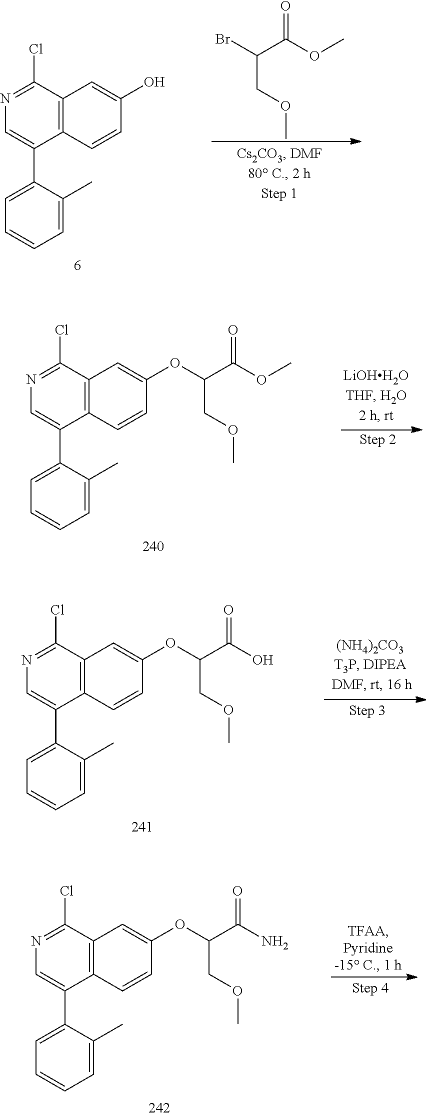

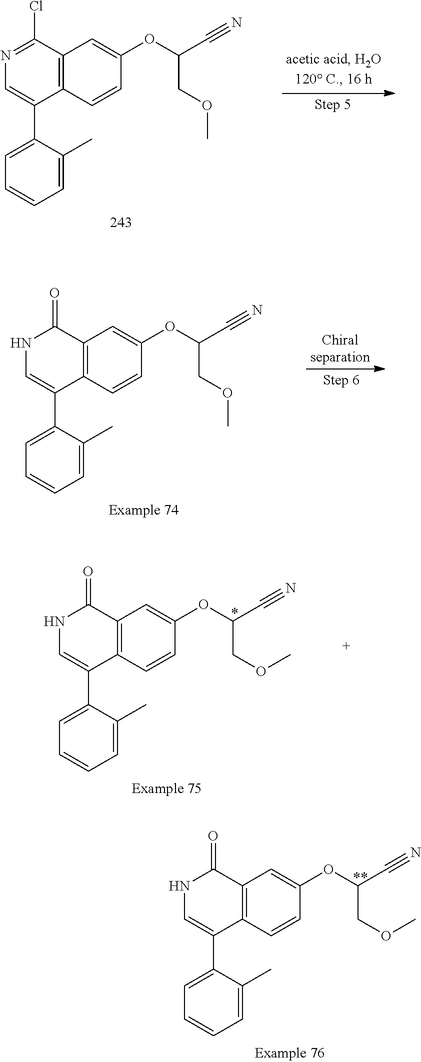 [SMILES: COCC(C#N)Oc1ccc2c(-c3ccccc3C)cnc(Cl)c2c1 ; COCC(C#N)Oc1ccc2c(-c3ccccc3C)c[nH]c(=O)c2c1 ; COCC(C#N)Oc1ccc2c(-c3ccccc3C)c[nH]c(=O)c2c1 ; COCC(C#N)Oc1ccc2c(-c3ccccc3C)c[nH]c(=O)c2c1]

Synthesis of methyl 2-((1-chloro-4-(o-tolyl)isoquinolin-7-yl)oxy)-3-methoxypropanoate, 240 [Step 1]: To a stirred solution of 1-chloro-4-(o-tolyl)isoquinolin-7-ol (6, 250 mg, 0.9 mmol) in DMF (3 mL) was added K₂CO₃ (255 mg, 1.9 mmol) and methyl 2-bromo-3-methoxypropanoate (175 mg, 0.9 mmol). The reaction mixture was stirred at 80° C. for 2 h. The reaction mixture was quenched with ice cold water and extracted with EtOAc. The combined organic layers were washed with water, brine, dried over anhydrous Na₂SO₄, filtered, and concentrated under reduced pressure. The product was purified by flash chromatography to afford methyl 2-((1-chloro-4-(o-tolyl)isoquinolin-7-yl)oxy)-3-methoxypropanoate (240, 280 mg). LCMS (ESI) Calcd. for C₂₁H₂₀ClNO₄: 385, found [M+H]⁺=386. ¹H NMR (400 MHz, DMSO-d₆) δ 8.06 (s, 1H), 7.55 (br s, 2H), 7.42 (br s, 2H), 7.35 (br s, 2H), 7.25 (br s, 1H), 5.46 (br s, 1H), 3.93 (br s, 1H), 3.86 (br s, 1H), 3.72 (s, 3H), 3.35 (s, 3H), 1.98 (s, 3H).

Synthesis of 2-((1-chloro-4-(o-tolyl)isoquinolin-7-yl)oxy)-3-methoxypropanoic acid, 241 [Step 2]: To a stirred solution of methyl 2-((1-chloro-4-(o-tolyl)isoquinolin-7-yl)oxy)-3-methoxypropanoate (240, 290 mg, 0.7 mmol) in THF (4 mL) was added an aq. solution of LiOH·H₂O (65 mg, 1.5 mmol) dropwise. The reaction mixture was stirred at ambient temperature for 2 h. The reaction mixture was concentrated under reduced pressure. The reaction mixture was diluted with water, acidified with citric acid, and extracted with EtOAc. The combined organic layers were washed with brine, dried over anhydrous Na₂SO₄, filtered, and concentrated under reduced pressure to afford 2-((1-chloro-4-(o-tolyl)isoquinolin-7-yl)oxy)-3-methoxypropanoic acid (241, 250 mg). LCMS (ESI) Calcd. for C₂₀H₁₈ClNO₄: 371, found [M+H]⁺=372.

Synthesis of 2-((1-chloro-4-(o-tolyl)isoquinolin-7-yl)oxy)-3-methoxypropanamide, 242 [Step 3]: To a stirred solution of 2-((1-chloro-4-(o-tolyl)isoquinolin-7-yl)oxy)-3-methoxypropanoic acid (241, 280 mg, 0.8 mmol) in DMF (4 mL) was added DIPEA (0.7 mL, 3.8 mmol) and (NH₄)₂CO₃ (360 mg, 3.8 mmol). T₃P (0.7 mL, 1.1 mmol, 50% in EtOAc) was added to the reaction mixture at 0° C. The reaction mixture was allowed to warm to ambient temperature and stirred for 16 h. The reaction mixture was quenched with ice cold water and extracted with EtOAc. The combined organic layers were washed with brine, dried over anhydrous Na₂SO₄, filtered, and concentrated under reduced pressure. The product was purified by flash chromatography to afford 2-((1-chloro-4-(o-tolyl)isoquinolin-7-yl)oxy)-3-methoxypropanamide (242, 210 mg). LCMS (ESI) Calcd. for C₂₀H₁₉C₁N₂O₃: 370, found [M+H]⁺=371. ¹H NMR (400 MHz, DMSO-d₆) δ 8.05 (s, 1H), 7.82-7.80 (d, 1H), 7.61-7.55 (m, 2H), 7.49 (m, 1H), 7.43-7.41 (m, 2H), 7.37-7.33 (m, 2H), 7.25-7.23 (d, 1H), 5.01-4.98 (m, 1H), 3.84-3.74 (m, 2H), 3.33 (s, 3H), 1.98 (s, 3H).

Synthesis of 2-((1-chloro-4-(o-tolyl)isoquinolin-7-yl)oxy)-3-methoxypropanenitrile, 243 [Step 4]: To a solution of 2-((1-chloro-4-(o-tolyl)isoquinolin-7-yl)oxy)-3-methoxypropanamide (242, 200 mg, 0.6 mmol) in pyridine (5 mL) was added dropwise trifluoroacetic anhydride (0.3 mL, 2.2 mmol) at −15° C. The reaction mixture was stirred at −15° C. for 1 h. The reaction mixture was concentrated under reduced pressure. The reaction mixture was cooled to 0° C., quenched with aq. NaHCO₃, and extracted with EtOAc. The combined organic layers were washed with brine, dried over anhydrous Na₂SO₄, filtered, and concentrated under reduced pressure to afford 2-((1-chloro-4-(o-tolyl)isoquinolin-7-yl)oxy)-3-methoxypropanenitrile (243, 180 mg). LCMS (ESI) Calcd. for C₂₀H₁₇ClN₂O₂: 352, found [M+H]⁺=353. ¹H NMR (400 MHz, DMSO-d₆) δ 8.13 (s, 1H), 7.89 (d, 1H), 7.64-7.60 (m, 1H), 7.45-7.36 (m, 4H), 7.27-7.25 (m, 1H), 5.98-5.96 (m, 1H), 3.95-3.94 (m, 2H), 3.44 (s, 3H), 1.98 (s, 3H).

Synthesis of 3-methoxy-2-((1-oxo-4-(o-tolyl)-1,2-dihydroisoquinolin-7-yl)oxy)propanenitrile, Example 74 [Step 5]: To a stirred solution of 2-((1-chloro-4-(o-tolyl)isoquinolin-7-yl)oxy)-3-methoxypropanenitrile (243, 170 mg, 0.5 mmol) in acetic acid (6.4 mL, 111 mmol), was added water (2.1 mL, 118 mmol). The reaction mixture was stirred at 120° C. for 16 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford 3-methoxy-2-((1-oxo-4-(o-tolyl)-1,2-dihydroisoquinolin-7-yl)oxy)propanenitrile (Example 74, 80 mg). LCMS (ESI) Calcd. for C₂₀H₁₈ClN₂O₃: 334, found [M+H]⁺=335.

Synthesis of chiral 3-methoxy-2-((1-oxo-4-(o-tolyl)-1,2-dihydroisoquinolin-7-yl)oxy)propanenitrile, Examples 75 and 76 [Step 6]: 3-methoxy-2-((1-oxo-4-(o-tolyl)-1,2-dihydroisoquinolin-7-yl)oxy)propanenitrile (80 mg) was purified by normal phase chiral prep-HPLC and lyophilized. The first product was isolated as 3-methoxy-2-((1-oxo-4-(o-tolyl)-1,2-dihydroisoquinolin-7-yl)oxy)propanenitrile, Peak 1 (Example 75, 50 mg) and the second product as 3-methoxy-2-((1-oxo-4-(o-tolyl)-1,2-dihydroisoquinolin-7-yl)oxy)propanenitrile, Peak 2 (Example 76, 30 mg). The absolute stereochemistry for these Examples was not determined.

Example 75: 3-methoxy-2-((1-oxo-4-(o-tolyl)-1,2-dihydroisoquinolin-7-yl)oxy)propanenitrile, Peak 1: LCMS (ESI) Calcd. for C₂₀H₁₈ClN₂O₃: 334, found [M+H]⁺=335. ¹H NMR (400 MHz, DMSO-d₆) δ 11.47-11.45 (m, 1H), 7.93 (s, 1H), 7.40-7.38 (m, 1H), 7.36-7.35 (m, 2H), 7.29-7.28 (m, 1H), 7.22-7.20 (m, 1H), 6.99-6.97 (m, 2H), 5.78 (br s, 1H), 3.89 (br s, 2H), 3.42 (s, 3H), 2.04 (s, 3H).

Example 76: 3-methoxy-2-((1-oxo-4-(o-tolyl)-1,2-dihydroisoquinolin-7-yl)oxy)propanenitrile, Peak 2: LCMS (ESI) Calcd. for C₂₀H₁₈ClN₂O₃: 334, found [M+H]⁺=335. ¹H NMR (400 MHz, DMSO-d₆) δ 11.47 (br s, 1H), 7.93 (s, 1H), 7.40-7.38 (m, 1H), 7.36-7.35 (m, 2H), 7.29-7.28 (m, 1H), 7.22-7.20 (m, 1H), 6.99-6.97 (m, 2H), 5.78 (m, 1H), 3.89 (m, 2H), 3.42 (s, 3H), 2.05 (s, 3H).

Chiral prep-HPLC: Chiral separation was performed on an Agilent 1200 series instrument. Column was a Chiralpak IG (250×21 mm), 5 μm, operating at ambient temperature with flow rate of 21.0 mL/min. mobile phase: 70% hexanes, 15% CH₂Cl₂, and 15% ethanol, held isocratic for up to 22 min. with detection at 284 nm wavelength.

### Examples 77-78: Synthesis of (S)-1-((R)-2-((1-oxo-4-(o-tolyl)-1,2-dihydroisoquinolin-7-yl)oxy)propanoyl)piperidine-3-carbonitrile and (R)-7-((1-morpholino-1-oxopropan-2-yl)oxy)-4-(o-tolyl)isoquinolin-1(2H)-one

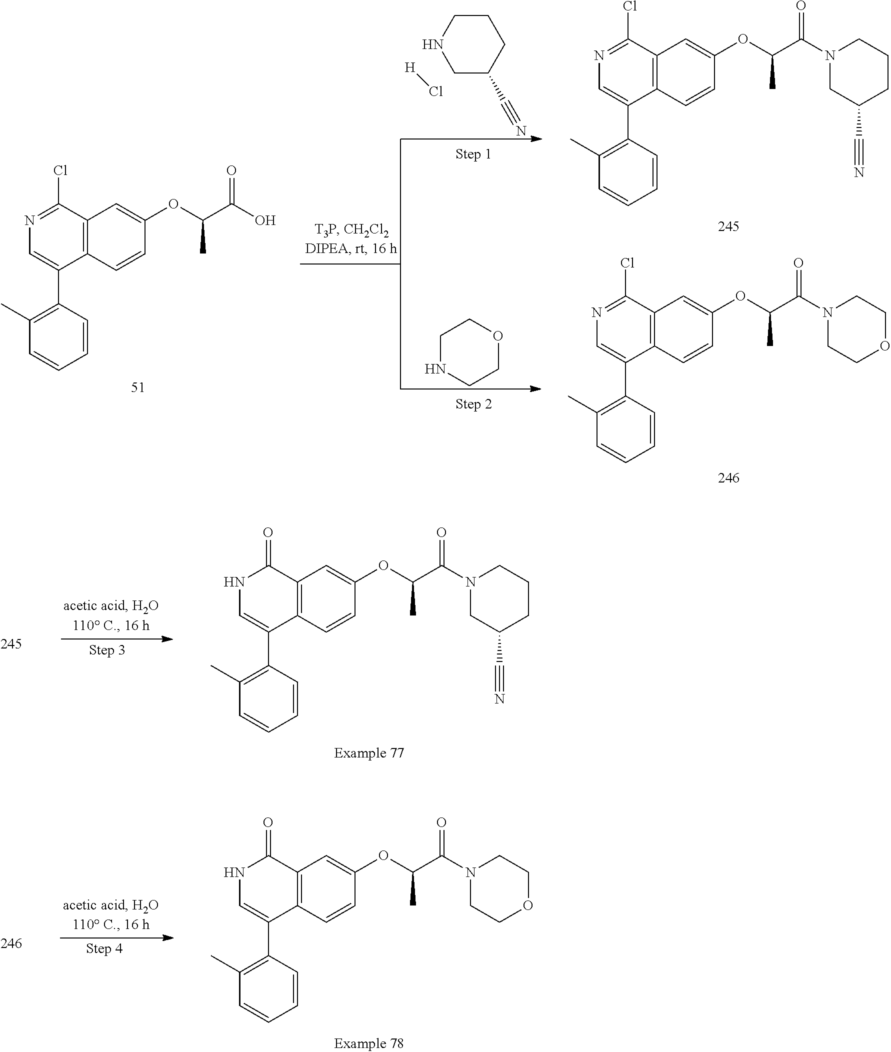

Synthesis of (S)-1-((R)-2-((1-chloro-4-(o-tolyl)isoquinolin-7-yl)oxy)propanoyl)piperidine-3-carbonitrile, 245 [Step 1]: To a stirred solution of (R)-2-((1-chloro-4-(o-tolyl)isoquinolin-7-yl)oxy)propanoic acid (51, 130 mg, 0.4 mmol) in CH₂Cl₂ (4 mL) was added (S)-piperidine-3-carbonitrile.HCl (85 mg, 0.6 mmol) followed by DIPEA (0.3 mL, 1.9 mmol). T₃P (0.3 mL, 0.6 mmol, 50% in EtOAc) was added to the reaction mixture at 0° C. The reaction mixture was allowed to warm to ambient temperature and stirred for 16 h. The reaction mixture was diluted with CH₂Cl₂ and washed with water, brine. The combined organic layers were dried over anhydrous Na₂SO₄, filtered, and concentrated under reduced pressure to afford (S)-1-((R)-2-((1-chloro-4-(o-tolyl)isoquinolin-7-yl)oxy)propanoyl)piperidine-3-carbonitrile (245, 152 mg). LCMS (ESI) Calcd. for C₂₅H₂₄C₁N₃O₂: 433, found [M+H]⁺=434.

Synthesis of (R)-2-((1-chloro-4-(o-tolyl)isoquinolin-7-yl)oxy)-1-morpholinopropan-1-one, 246 [Step 2]: To a stirred solution of (R)-2-((1-chloro-4-(o-tolyl)isoquinolin-7-yl)oxy)propanoic acid (51, 120 mg, 0.4 mmol) in CH₂Cl₂ (4 mL) was added morpholine (0.04 mL, 0.5 mmol) and DIPEA (0.2 mL, 1.7 mmol). T₃P (0.3 mL, 0.5 mmol, 50% in EtOAc)) was added to the reaction mixture at 0° C. The reaction mixture was allowed to warm to ambient temperature and stirred for 16 h. The reaction mixture was diluted with CH₂Cl₂ and washed with water, brine. The combined organic layers were dried over anhydrous Na₂SO₄, filtered, and concentrated under reduced pressure to afford (R)-2-((1-chloro-4-(o-tolyl)isoquinolin-7-yl)oxy)-1-morpholinopropan-1-one (246, 125 mg). LCMS (ESI) Calcd. for C₂₃H₂₃C₁N₂O₃: 410, found [M+H]⁺=411.

Synthesis of (S)-1-((R)-2-((1-oxo-4-(o-tolyl)-1,2-dihydroisoquinolin-7-yl)oxy)propanoyl)piperidine-3-carbonitrile, Example 77 [Step 3]: To a stirred solution of (S)-1-((R)-2-((1-chloro-4-(o-tolyl)isoquinolin-7-yl)oxy)propanoyl)piperidine-3-carbonitrile (245, 152 mg, 0.4 mmol) in acetic acid (3.0 mL, 52.5 mmol) was added water (0.6 mL, 35.0 mmol). The reaction mixture was heated to reflux for 16 h. The reaction mixture was concentrated under reduced pressure and diluted in EtOAc (50 mL). The organic layer was washed with water, brine, dried over anhydrous Na₂SO₄, filtered, and concentrated under reduced pressure. The product was purified by reverse phase prep-HPLC and lyophilized to afford (S)-1-((R)-2-((1-oxo-4-(o-tolyl)-1,2-dihydroisoquinolin-7-yl)oxy)propanoyl)piperidine-3-carbonitrile (Example 77, 40 mg). LCMS (ESI) Calcd. for C₂₅H₂₅N₃O₃: 415, found [M+H]⁺=416. ¹H NMR (400 MHz, DMSO-d₆) δ 11.31 (br s, 1H), 7.67-7.56 (m, 1H), 7.34-7.17 (m, 5H), 6.91-6.89 (d, 2H), 5.58-5.44 (m, 1H), 4.11-3.19 (m, 5H), 2.07 (br s, 3H), 1.88-1.62 (m, 2H), 1.48 (br s, 4H).

Synthesis of (R)-7-((1-morpholino-1-oxopropan-2-yl)oxy)-4-(o-tolyl)isoquinolin-1(2H)-one, Example 78 [Step 4]: To a stirred solution of (R)-2-((1-chloro-4-(o-tolyl)isoquinolin-7-yl)oxy)-1-morpholinopropan-1-one (246, 125 mg, 0.3 mmol) in acetic acid (2.6 mL, 45.6 mmol) was added water (0.6 mL, 30.4 mmol). The reaction mixture was heated to reflux for 12 h. The reaction mixture was concentrated under reduced pressure and diluted in EtOAc (50 mL). The combined organic layers were washed with water, brine, dried over anhydrous Na₂SO₄, filtered, and concentrated under reduced pressure. The product was purified by reverse phase prep-HPLC and lyophilized to afford (R)-7-((1-morpholino-1-oxopropan-2-yl)oxy)-4-(o-tolyl)isoquinolin-1(2H)-one (Example 78, 48 mg). LCMS (ESI) Calcd. for C₂₃H₂₄N₂O₄: 392, found [M+H]⁺=393. ¹H NMR (400 MHz, DMSO-d₆) δ 11.31 (s, 1H), 7.62 (br s, 1H), 7.35 (br s, 2H), 7.28-7.18 (m, 3H), 6.91-6.89 (m, 2H), 5.45 (br s, 1H), 3.77 (br s, 2H), 3.61-3.55 (m, 5H), 3.42 (br s, 1H), 2.04 (s, 3H), 1.46 (d, 3H).

### Example 79: Synthesis of (R)-4-(2-chloro-4-fluorophenyl)-7-((1-morpholino-1-oxopropan-2-yl)oxy)isoquinolin-1(2)-one

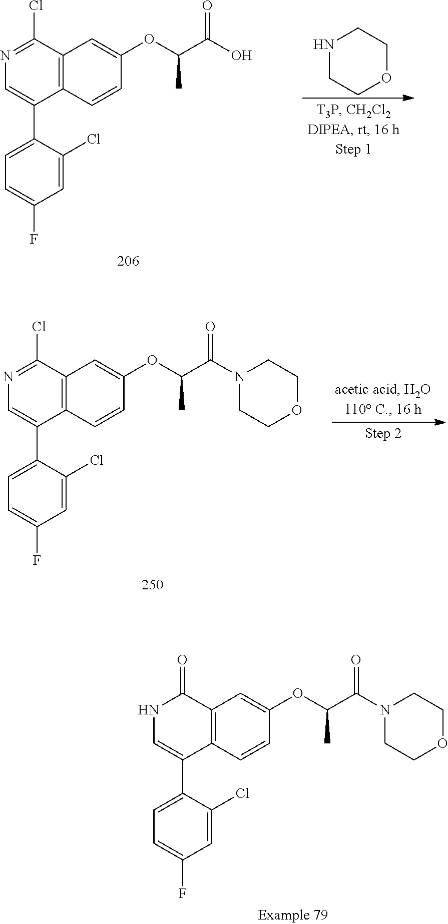 [SMILES: C[C@@H](Oc1ccc2c(-c3ccc(F)cc3Cl)cnc(Cl)c2c1)C(=O)O ; C1COCCN1 ; C[C@@H](Oc1ccc2c(-c3ccc(F)cc3Cl)cnc(Cl)c2c1)C(=O)N1CCOCC1 ; C[C@@H](Oc1ccc2c(-c3ccc(F)cc3Cl)c[nH]c(=O)c2c1)C(=O)N1CCOCC1]

Synthesis of (R)-2-((1-chloro-4-(2-chloro-4-fluorophenyl)isoquinolin-7-yl)oxy)-1-morpholinopropan-1-one, 250 [Step 1]: To a stirred solution of (R)-2-((1-chloro-4-(2-chloro-4-fluorophenyl)isoquinolin-7-yl)oxy)propanoic acid (206, 130 mg, 0.3 mmol) in CH₂Cl₂ (4 mL) was added morpholine (0.04 mL, 0.5 mmol) and DIPEA (0.2 mL, 1.7 mmol). T₃P (0.3 mL, 0.5 mmol, 50% in EtOAc) was added to the reaction mixture at 0° C. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (R)-2-((1-chloro-4-(2-chloro-4-fluorophenyl)isoquinolin-7-yl)oxy)-1-morpholinopropan-1-one (250, 140 mg). LCMS (ESI) Calcd. for C₂₂H₁₉C₁₂FN₂O₃: 448, found [M+H]⁺=449.

Synthesis of (R)-4-(2-chloro-4-fluorophenyl)-7-((1-morpholino-1-oxopropan-2-yl)oxy)isoquinolin-1(2H)-one, Example 79 [Step 2]: To a stirred solution of (R)-2-((1-chloro-4-(2-chloro-4-fluorophenyl)isoquinolin-7-yl)oxy)-1-morpholinopropan-1-one (250, 200 mg, 0.4 mmol) in acetic acid (3.8 mL, 67.1 mmol) was added water (0.80 mL, 44.7 mmol). The reaction mixture was heated to reflux for 16 h. The reaction mixture was concentrated under reduced pressure and extracted in EtOAc. The combined organic layers were washed with water, brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-morpholino-1-oxopropan-2-yl)oxy)isoquinolin-1(2H)-one (Example 79, 52 mg). LCMS (ESI) Calcd. for C₂₂H₂₀ClFN₂O₄: 430, found [M+H]⁺=431. ¹H NMR (400 MHz, DMSO-d₆) δ 11.41 (br s, 1H), 7.61-7.60 (m, 2H), 7.50-7.45 (m, 1H), 7.36-7.32 (m, 1H), 7.26-7.24 (m, 1H), 7.00-6.98 (m, 1H), 6.95 (d, 1H), 5.47-5.44 (m, 1H), 3.77-3.55 (m, 8H), 1.46 (d, 3H).

### Examples 80-81: Synthesis of (R)-7-((1-(4,4-difluoropiperidin-1-yl)-1-oxopropan-2-yl)oxy)-4-(o-tolyl)isoquinolin-1(2H)-one and (R)-7-((1-(3,3-difluoropiperidin-1-yl)-1-oxopropan-2-yl)oxy)-4-(o-tolyl)isoquinolin-1(2H)-one

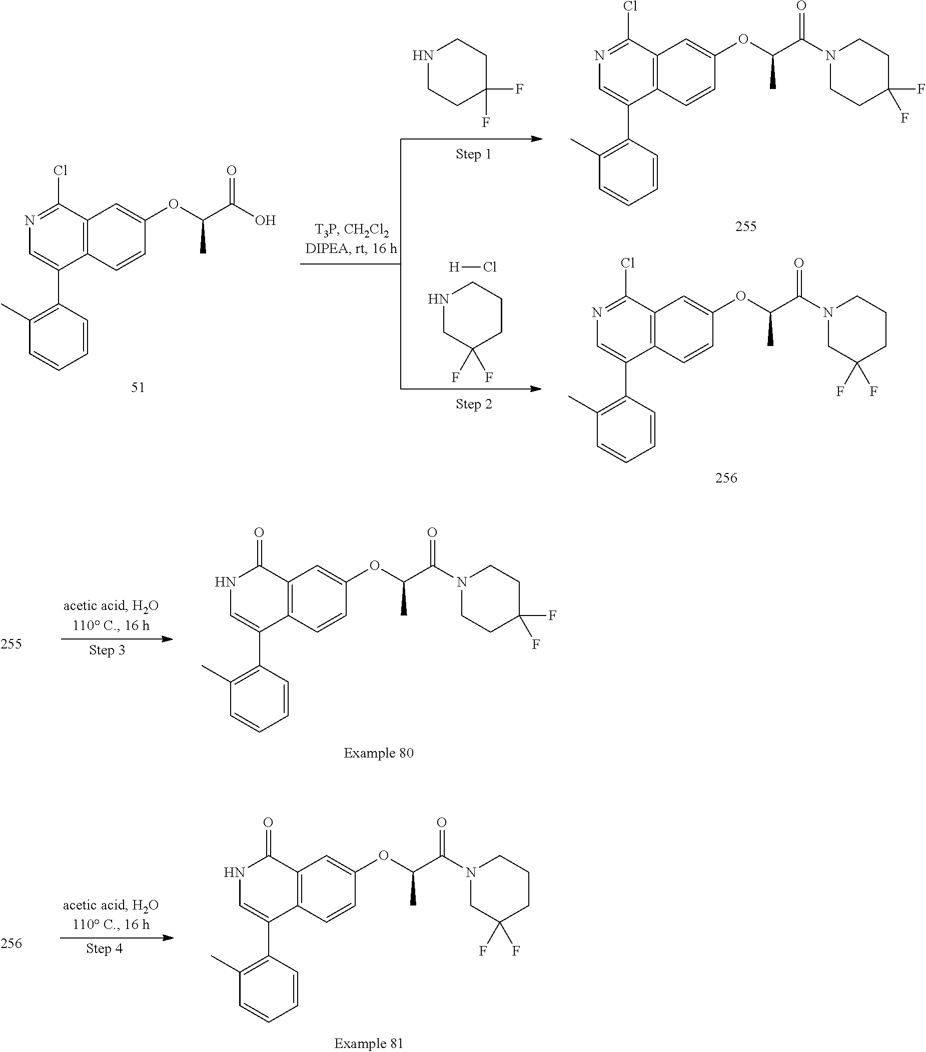

Synthesis of (R)-2-((1-chloro-4-(o-tolyl)isoquinolin-7-yl)oxy)-1-(4,4-difluoropiperidin-1-yl)propan-1-one, 255 [Step 1]: To a stirred solution of (R)-2-((1-chloro-4-(o-tolyl)isoquinolin-7-yl)oxy)propanoic acid (51, 105 mg, 0.3 mmol) in CH₂Cl₂ (4 mL) was added DIPEA (0.3 mL, 1.5 mmol) followed by 4,4-difluoropiperidine (45 mg, 0.4 mmol). T₃P (0.3 mL, 0.5 mmol, 50% in EtOAc) was added to the reaction mixture at 0° C. The reaction mixture was allowed to warm to ambient temperature and stirred for 16 h. The reaction mixture was quenched with ice cold water and extracted with CH₂Cl₂. The combined organic layers were washed with brine, dried over anhydrous Na₂SO₄, filtered, and concentrated under reduced pressure to afford (R)-2-((1-chloro-4-(o-tolyl)isoquinolin-7-yl)oxy)-1-(4,4-difluoropiperidin-1-yl)propan-1-one (255, 100 mg). LCMS (ESI) Calcd. for C₂₄H₂₃ClF₂N₂O₂: 444, found [M+H]⁺=445.

Synthesis of (R)-2-((1-chloro-4-(o-tolyl)isoquinolin-7-yl)oxy)-1-(3,3-difluoropiperidin-1-yl)propan-1-one, 256 [Step 2]: To a stirred solution of (R)-2-((1-chloro-4-(o-tolyl)isoquinolin-7-yl)oxy)propanoic acid (51, 120 mg, 0.4 mmol) in CH₂Cl₂ (4 mL) was added 3,3-difluoropiperidine hydrochloride (85 mg, 0.5 mmol) followed by DIPEA (0.24 mL, 1.8 mmol). T₃P (0.3 mL, 0.5 mmol, 50% in EtOAc) was added to the reaction mixture at 0° C. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (R)-2-((1-chloro-4-(o-tolyl)isoquinolin-7-yl)oxy)-1-(3,3-difluoropiperidin-1-yl)propan-1-one (256, 140 mg). LCMS (ESI) Calcd. for C₂₄H₂₃ClF₂N₂O₂: 444, found [M+H]⁺=445.

Synthesis of (R)-7-((1-(4,4-difluoropiperidin-1-yl)-1-oxopropan-2-yl)oxy)-4-(o-tolyl)isoquinolin-1(2H)-one, Example 80 [Step 3]: To a stirred solution of (R)-2-((1-chloro-4-(o-tolyl)isoquinolin-7-yl)oxy)-1-(4,4-difluoropiperidin-1-yl)propan-1-one (255, 100 mg, 0.2 mmol) in acetic acid (2.8 mL, 49.4 mmol) was added water (0.8 mL, 45.0 mmol). The reaction mixture was heated to 120° C. for 16 h. The reaction mixture was concentrated under reduced pressure. The reaction mixture was quenched with ice cold water and extracted with EtOAc. The combined organic layers were washed with brine, dried over anhydrous Na₂SO₄, filtered, and concentrated under reduced pressure. The product was purified by reverse phase prep-HPLC and lyophilized to afford (R)-7-((1-(4,4-difluoropiperidin-1-yl)-1-oxopropan-2-yl)oxy)-4-(o-tolyl)isoquinolin-1(2H)-one (Example 80, 30 mg). LCMS (ESI) Calcd. for C₂₄H₂₄F₂N₂O₃: 426, found [M+H]⁺=427. ¹H NMR (400 MHz, DMSO-d₆) δ 11.32 (br s, 1H), 7.58 (br s, 1H), 7.34 (br s, 2H), 7.25-7.23 (m, 2H), 7.19-7.17 (m, 1H), 6.92 (br s, 2H), 5.51 (br s, 1H), 3.94 (br s, 2H), 3.54 (br s, 1H), 3.31 (br s, 1H), 2.32-1.82 (m, 7H), 1.46 (d, 3H).

Synthesis of (R)-7-((1-(3,3-difluoropiperidin-1-yl)-1-oxopropan-2-yl)oxy)-4-(o-tolyl)isoquinolin-1(2H)-one, Example 81 [Step 4]: A stirred solution of (R)-2-((1-chloro-4-(o-tolyl)isoquinolin-7-yl)oxy)-1-(3,3-difluoropiperidin-1-yl)propan-1-one (256, 140 mg, 0.3 mmol) in acetic acid (2.7 mL, 47.2 mmol) and water (0.6 mL, 31.5 mmol) was heated to reflux for 12 h. The reaction mixture was concentrated under reduced pressure. The residue was dissolved in EtOAc, washed with water, brine, dried over anhydrous Na₂SO₄, and concentrated under reduced pressure. The product was purified by reverse phase prep-HPLC and lyophilized to afford (R)-7-((1-(3,3-difluoropiperidin-1-yl)-1-oxopropan-2-yl)oxy)-4-(o-tolyl)isoquinolin-1(2H)-one (Example 81, 28 mg). LCMS (ESI) Calcd. for C₂₄H₂₄F₂N₂O₃: 426, found [M+H]⁺=427. ¹H NMR (400 MHz, DMSO-d₆) δ 11.30 (br s, 1H), 7.61 (br s, 1H), 7.34-7.17 (m, 5H), 6.88 (br s, 2H), 5.50 (br s, 1H), 3.99-3.87 (m, 3H), 3.69-3.50 (m, 1H), 2.08-2.03 (m, 5H), 1.86-1.68 (m, 2H), 1.46 (d, 3H).

### Example 82: Synthesis of (S′)-1-((R)-2-((4-(2-chloro-4-fluorophenyl)-1-oxo-1,2-dihydroisoquinolin-7-yl)oxy)propanoyl)piperidine-3-carboxamide

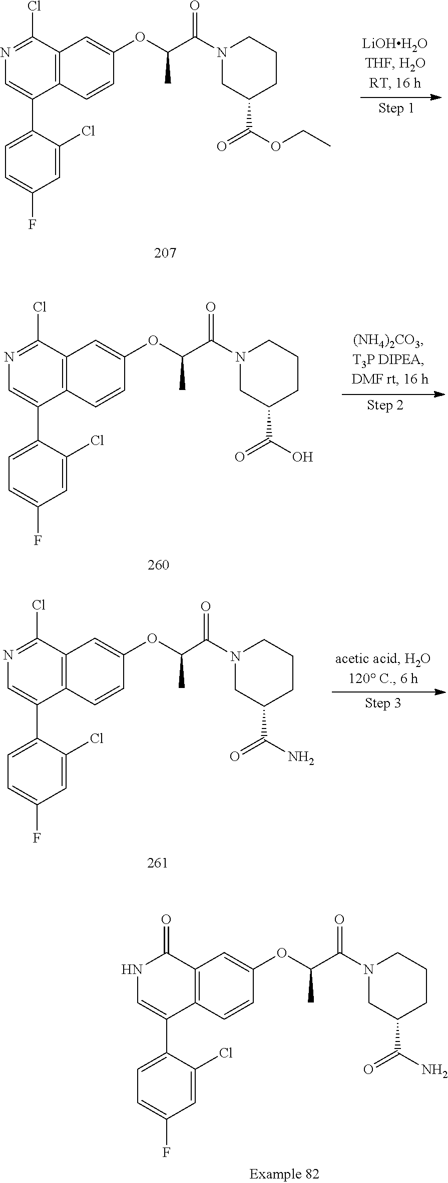 [SMILES: CCOC(=O)[C@H]1CCCN(C(=O)[C@@H](C)Oc2ccc3c(-c4ccc(F)cc4Cl)cnc(Cl)c3c2)C1 ; C[C@@H](Oc1ccc2c(-c3ccc(F)cc3Cl)cnc(Cl)c2c1)C(=O)N1CCC[C@H](C(=O)O)C1 ; C[C@@H](Oc1ccc2c(-c3ccc(F)cc3Cl)cnc(Cl)c2c1)C(=O)N1CCC[C@H](C(N)=O)C1 ; C[C@@H](Oc1ccc2c(-c3ccc(F)cc3Cl)c[nH]c(=O)c2c1)C(=O)N1CCC[C@H](C(N)=O)C1]

Synthesis of (S)-1-((R)-2-((1-chloro-4-(2-chloro-4-fluorophenyl)isoquinolin-7-yl)oxy)propanoyl)piperidine-3-carboxylic acid, 260 [Step 1]: To a stirred solution of ethyl (S)-1-((R)-2-((1-chloro-4-(2-chloro-4-fluorophenyl)isoquinolin-7-yl)oxy)propanoyl)piperidine-3-carboxylate (207, 800 mg, 1.5 mmol) in THF (6 mL) and water (2 mL) was added LiOH·H₂O (130 mg, 3 mmol). The reaction mixture was allowed to stir at ambient temperature for 16 h. The reaction mixture was concentrated under reduced pressure. The reaction mixture was partitioned between water and EtOAc, acidified with 1N HCl, and extracted with EtOAc. The combined organic layers were washed with brine, dried over anhydrous Na₂SO₄, filtered, and concentrated under reduced pressure to afford (S)-1-((R)-2-((1-chloro-4-(2-chloro-4-fluorophenyl)isoquinolin-7-yl)oxy)propanoyl)piperidine-3-carboxylic acid (260, 680 mg). LCMS (ESI) Calcd. for C₂₄H₂₁Cl₂FN₂O₄: 490, found [M+H]⁺=491.

Synthesis (S)-1-((R)-2-((1-chloro-4-(2-chloro-4-fluorophenyl)isoquinolin-7-yl)oxy)propanoyl)piperidine-3-carboxamide, 261 [Step 2]: To a stirred solution of (S)-1-((R)-2-((1-chloro-4-(2-chloro-4-fluorophenyl)isoquinolin-7-yl)oxy)propanoyl)piperidine-3-carboxylic acid (260, 220 mg, 0.4 mmol) in DMF (3 mL) was added (NH₄)₂CO₃ (430 mg, 4.5 mmol) and DIPEA (0.4 mL, 2.2 mmol). T₃P (0.4 mL, 0.7 mmol, 50% in EtOAc) was added to the reaction mixture at 0° C. The reaction mixture was allowed to warm to ambient temperature and stirred for 16 h. The reaction mixture was quenched with ice cold water and extracted with EtOAc. The combined organic layers were washed with brine, dried over anhydrous Na₂SO₄, and concentrated under reduced pressure to afford (S)-1-((R)-2-((1-chloro-4-(2-chloro-4-fluorophenyl)isoquinolin-7-yl)oxy)propanoyl)piperidine-3-carboxamide (261, 210 mg). LCMS (ESI) Calcd. for C₂₄H₂₂Cl₂FN₃O₃: 489, found [M+H]⁺=490.

Synthesis of (S)-1-((R)-2-((4-(2-chloro-4-fluorophenyl)-1-oxo-1,2-dihydroisoquinolin-7-yl)oxy)propanoyl)piperidine-3-carboxamide, Example 82 [Step 3]: To a stirred solution of (S)-1-((R)-2-((1-chloro-4-(2-chloro-4-fluorophenyl)isoquinolin-7-yl)oxy)propanoyl)piperidine-3-carboxamide (261, 210 mg, 0.4 mmol) in acetic acid (3.7 mL, 64.2 mmol) was added water (0.8 mL, 42.8 mmol). The reaction mixture was heated to reflux for 12 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford (S)-1-((R)-2-((4-(2-chloro-4-fluorophenyl)-1-oxo-1,2-dihydroisoquinolin-7-yl)oxy)propanoyl)piperidine-3-carboxamide (Example 82, 42 mg). LCMS (ESI) Calcd. for C₂₄H₂₃ClFN₃O₄: 471, found [M+H]⁺=472. ¹H NMR (400 MHz, DMSO-d₆) δ 11.37 (br s, 1H), 7.61-7.59 (m, 2H), 7.48-7.46 (m, 1H), 7.36-7.32 (m, 2H), 7.26-7.24 (d, 1H), 7.00-6.81 (m, 3H), 5.50 (br s, 1H), 4.40 (br s, 1H), 4.10 (br s, 2H), 3.05 (br s, 1H), 2.57-1.59 (m, 5H), 1.46 (br s, 3H).

### Example 83: Synthesis of 2-((4-(4-fluoro-2-methylphenyl)-1-oxo-1,2-dihydroisoquinolin-7-yl)oxy)acetonitrile

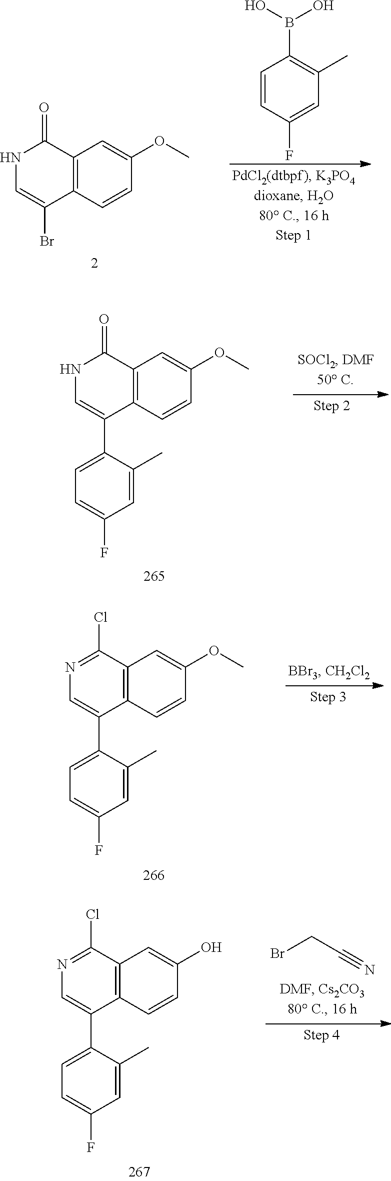

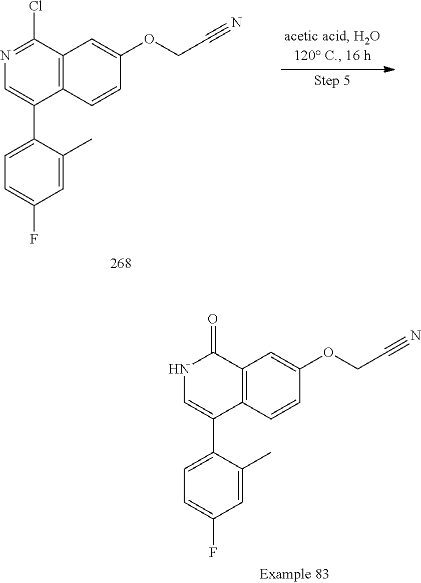 [SMILES: Cc1cc(F)ccc1-c1cnc(Cl)c2cc(OCC#N)ccc12 ; Cc1cc(F)ccc1-c1c[nH]c(=O)c2cc(OCC#N)ccc12]

Synthesis of 4-(4-fluoro-2-methylphenyl)-7-methoxyisoquinolin-1(2H)-one, 265 [Step 1]: To a stirred solution of 4-bromo-7-methoxyisoquinolin-1(2H)-one (2, 100 mg, 0.4 mmol) and (4-fluoro-2-methyl-phenyl)boronic acid (90 mg, 0.6 mmol) in 1,4-dioxane (4 mL) and water (1 mL) was added K₃PO₄ (420 mg, 2 mmol). The reaction mixture was degassed with argon for 10 min., and PdCl₂(dtbpf) (25 mg, 0.04 mmol) was added. The reaction mixture was heated to reflux for 16 h. The reaction mixture was filtered through celite. The filtrate was concentrated under reduced pressure. The product was dissolved in EtOAc, washed with water, brine, dried over anhydrous Na₂SO₄, filtered, and concentrated under reduced pressure. The product was purified by flash chromatography to afford 4-(4-fluoro-2-methylphenyl)-7-methoxyisoquinolin-1(2H)-one (265, 80 mg). LCMS (ESI) Calcd. for C₁₇H₁₄FNO₂: 283, found [M+H]⁺=284. ¹H NMR (400 MHz, DMSO-d₆) δ 11.38 (br s, 1H), 7.70 (br s, 1H), 7.28-7.20 (m, 3H), 7.13-7.08 (m, 1H), 6.91-6.88 (m, 2H), 3.86 (s, 3H), 2.03 (s, 3H).

Synthesis of 1-chloro-4-(4-fluoro-2-methylphenyl)-7-methoxyisoquinoline, 266 [Step 2]: To a stirred solution of 4-(4-fluoro-2-methylphenyl)-7-methoxyisoquinolin-1(2H)-one (265, 170 mg, 0.6 mmol) in SOCl₂ (2.2 mL, 30.0 mmol) was added DMF (0.05 mL, 0.6 mmol). The reaction mixture was heated at 50° C. for 16 h. The reaction mixture was concentrated under reduced pressure. The residue was quenched with ice-water and extracted with EtOAc. The combined organic layer was washed with water, brine, dried over anhydrous Na₂SO₄, and concentrated under reduced pressure to afford 1-chloro-4-(4-fluoro-2-methylphenyl)-7-methoxyisoquinoline (266, 120 mg). LCMS (ESI) Calcd. for C₁₅H₁₁N₃O₂: 301, found [M+H]⁺=302.

Synthesis of 1-chloro-4-(4-fluoro-2-methylphenyl)isoquinolin-7-ol, 267 [Step 3]: To a stirred solution of 1-chloro-4-(4-fluoro-2-methyl-phenyl)-7-methoxy-isoquinoline (266, 120 mg, 0.4 mmol) in CH₂Cl₂ (3 mL) was added dropwise BBr₃ (1.2 mL, 1.2 mmol, 1M in CH₂Cl₂) at 0° C. The reaction mixture was allowed to warm to ambient temperature and stirred for 16 h. The reaction mixture was concentrated under reduced pressure, cooled to 0° C., quenched with MeOH and cold water, and extracted with EtOAc. The combined organic layers were washed with brine, dried over anhydrous Na₂SO₄, filtered, and concentrated under reduced pressure. The product was purified by flash chromatography to afford 1-chloro-4-(4-fluoro-2-methylphenyl)isoquinolin-7-ol (267, 110 mg). LCMS (ESI) Calcd. for C₁₆H₁₁ClFNO: 287, found [M+H]⁺=288.

Synthesis of 2-((1-chloro-4-(4-fluoro-2-methylphenyl)isoquinolin-7-yl)oxy)acetonitrile, 268 [Step 4]: To a stirred solution of 2-bromoacetonitrile (65 mg, 0.5 mmol) and 1-chloro-4-(4-fluoro-2-methylphenyl)isoquinolin-7-ol (267, 125 mg, 0.4 mmol) in DMF (2 mL) was added Cs₂CO₃ (430 mg, 1.3 mmol) at ambient temperature. The reaction mixture was gradually heated up to 100° C. and stirred for 16 h. The reaction mixture was quenched with water and extracted with EtOAc. The combined organic layers were washed with water, brine, dried over anhydrous Na₂SO₄, filtered, and concentrated under reduced pressure to afford 2-((1-chloro-4-(4-fluoro-2-methylphenyl)isoquinolin-7-yl)oxy)acetonitrile (268, 90 mg). LCMS (ESI) Calcd. for C₁₈H₁₂ClFN₂O: 326, found [M+H]⁺=327.

Synthesis of 2-((4-(4-fluoro-2-methylphenyl)-1-oxo-1,2-dihydroisoquinolin-7-yl)oxy)acetonitrile, Example 83 [Step 5]: To a stirred solution of 2-((1-chloro-4-(4-fluoro-2-methylphenyl)isoquinolin-7-yl)oxy)acetonitrile (268, 80 mg, 0.2 mmol) in acetic acid (2.1 mL, 36.7 mmol) was added water (0.44 mL, 24.5 mmol). The reaction mixture was stirred at 110° C. for 16 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford 2-((4-(4-fluoro-2-methylphenyl)-1-oxo-1,2-dihydroisoquinolin-7-yl)oxy)acetonitrile (Example 83, 33 mg). LCMS (ESI) Calcd. for C₁₈H₁₃FN₂O₂: 308, found [M+H]⁺=309. ¹H NMR (400 MHz, DMSO-d₆) δ 11.48 (br s, 1H), 7.86 (br s, 1H), 7.38-7.35 (m, 1H), 7.26-7.20 (m, 2H), 7.13-7.09 (m, 1H), 6.98-6.96 (m, 2H), 5.32 (s, 2H), 2.07 (s, 3H).

### Example 84: Synthesis of (R)-4-(2-chloro-4-fluorophenyl)-7-((1-(3,3-difluoropiperidin-1-yl)-1-oxopropan-2-yl)oxy)isoquinolin-1(2H)-one

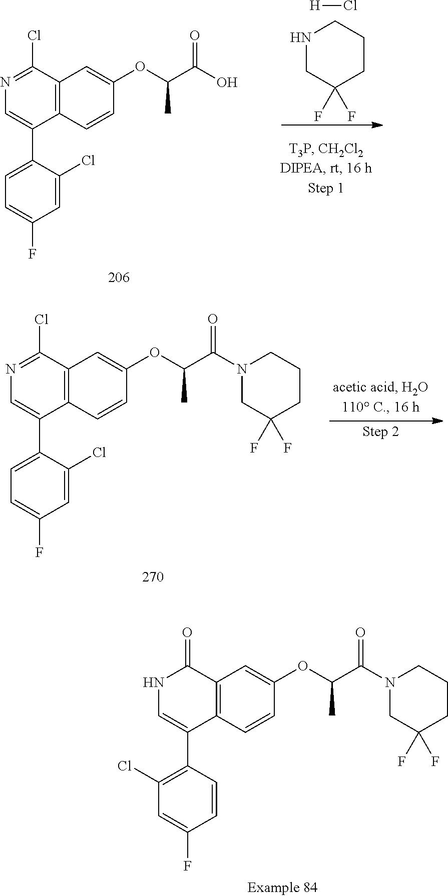

Synthesis of (R)-2-((1-chloro-4-(2-chloro-4-fluorophenyl)isoquinolin-7-yl)oxy)-1-(3,3-difluoropiperidin-1-yl)propan-1-one, 270 [Step 1]: To a stirred solution of (R)-2-((1-chloro-4-(2-chloro-4-fluorophenyl)isoquinolin-7-yl)oxy)propanoic acid (206, 125 mg, 0.3 mmol) in CH₂Cl₂ (4 mL) was added 3,3-difluoropiperidine hydrochloride (80 mg, 0.5 mmol) followed by DIPEA (0.2 mL, 1.6 mmol). T₃P (0.3 mL, 0.5 mmol, 50% in EtOAc) was added to the reaction mixture at 0° C. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (R)-2-((1-chloro-4-(2-chloro-4-fluorophenyl)isoquinolin-7-yl)oxy)-1-(3,3-difluoropiperidin-1-yl)propan-1-one (270, 140 mg). LCMS (ESI) Calcd. for C₂₃H₁₉Cl₂F₃N₂O₂: 482, found [M+H]⁺=483.

Synthesis of (R)-4-(2-chloro-4-fluorophenyl)-7-((1-(3,3-difluoropiperidin-1-yl)-1-oxopropan-2-yl)oxy)isoquinolin-1(2H)-one, Example 84 [Step 2]: To a stirred solution of (R)-2-((1-chloro-4-(2-chloro-4-fluorophenyl)isoquinolin-7-yl)oxy)-1-(3,3-difluoropiperidin-1-yl)propan-1-one (270, 140 mg, 0.3 mmol) in acetic acid (2.5 mL, 43.5 mmol) was added water (0.5 mL, 29.0 mmol). The reaction mixture was heated to reflux for 16 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-(3,3-difluoropiperidin-1-yl)-1-oxopropan-2-yl)oxy)isoquinolin-1(2H)-one (Example 84, 60 mg). LCMS (ESI) Calcd. for C₂₃H₂₀ClF₃N₂O₃: 464, found [M+H]⁺=465. ¹H NMR (400 MHz, DMSO-d₆) δ 11.39 (br s, 1H), 7.61-7.59 (m, 2H), 7.49-7.45 (t, 1H), 7.35-7.31 (t, 1H), 7.25-7.23 (m, 1H), 6.99 (m, 1H), 6.94-6.92 (d, 1H), 5.52 (br s, 1H), 3.98-3.69 (m, 4H), 2.08-1.62 (m, 4H), 1.46 (d, 3H).

### Examples 85-86: Synthesis of 7-(((2R)-1-(3-oxa-8-azabicyclo[3.2.1]octan-8-yl)-1-oxopropan-2-yl)oxy)-4-(o-tolyl)isoquinolin-1(2H)-one and 7-(((2R)-1-(8-azabicyclo[3.2.1]octan-8-yl)-1-oxopropan-2-yl)oxy)-4-(o-tolyl)isoquinolin-1(2H)-one

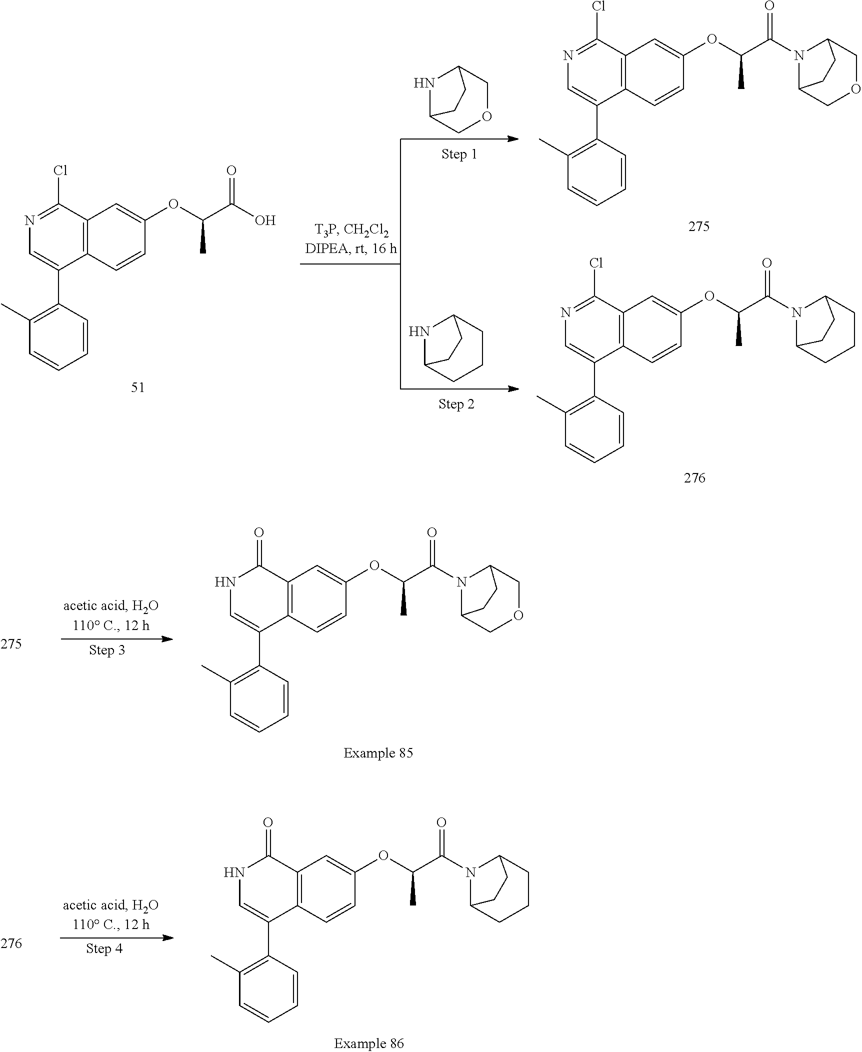

Synthesis of (2R)-1-(3-oxa-8-azabicyclo[3.2.1]octan-8-yl)-2-((1-chloro-4-(o-tolyl)isoquinolin-7-yl)oxy)propan-1-one, 275 [Step 1]: To a stirred solution of (R)-2-((1-chloro-4-(o-tolyl)isoquinolin-7-yl)oxy)propanoic acid (51, 120 mg, 0.4 mmol) in CH₂Cl₂ (4 mL) was added DIPEA (0.25 mL, 1.8 mmol) and 3-oxa-8-azabicyclo[3.2.1]octane (1, 60 mg, 0.5 mmol). T₃P (0.3 mL, 0.5 mmol, 50% in EtOAc) was added to the reaction mixture at 0° C. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (2R)-1-(3-oxa-8-azabicyclo[3.2.1]octan-8-yl)-2-((1-chloro-4-(o-tolyl)isoquinolin-7-yl)oxy)propan-1-one (275, 138 mg). LCMS (ESI) Calcd. for C₂₅H₂₅C₁N₂O₃: 436, found [M+H]⁺=437.

Synthesis of (2R)-1-(8-azabicyclo[3.2.1]octan-8-yl)-2-((1-chloro-4-(o-tolyl)isoquinolin-7-yl)oxy)propan-1-one, 270 [Step 2]: To a stirred solution of (R)-2-((1-chloro-4-(o-tolyl)isoquinolin-7-yl)oxy)propanoic acid (51, 110 mg, 0.3 mmol) in CH₂Cl₂ (4 mL) was added 8-azabicyclo[3.2.1]octane (55 mg, 0.5 mmol) and DIPEA (0.2 mL, 1.6 mmol). T₃P (0.3 mL, 0.5 mmol, 50% in EtOAc) was added to the reaction mixture at 0° C. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (2R)-1-(8-azabicyclo[3.2.1]octan-8-yl)-2-((1-chloro-4-(o-tolyl)isoquinolin-7-yl)oxy)propan-1-one (270, 135 mg). LCMS (ESI) Calcd. for C₂₆H₂₇ClN₂O₂: 434, found [M+H]⁺=435.

Synthesis of 7-(((2R)-1-(3-oxa-8-azabicyclo[3.2.1]octan-8-yl)-1-oxopropan-2-yl)oxy)-4-(o-tolyl)isoquinolin-1(2H)-one, Example 85 [Step 3]: To a stirred solution of (2R)-1-(3-oxa-8-azabicyclo[3.2.1]octan-8-yl)-2-((1-chloro-4-(o-tolyl)isoquinolin-7-yl)oxy)propan-1-one (275, 135 mg, 0.3 mmol) in acetic acid (2.7 mL, 46.6 mmol) was added water (0.56 mL, 31.0 mmol). The reaction mixture was heated to reflux for 12 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford 7-(((2R)-1-(3-oxa-8-azabicyclo[3.2.1]octan-8-yl)-1-oxopropan-2-yl)oxy)-4-(o-tolyl)isoquinolin-1(2H)-one (Example 85, 45 mg). LCMS (ESI) Calcd. for C₂₅H₂₆N₂O₄: 418, found [M+H]⁺=419. ¹H NMR (400 MHz, DMSO-d₆) δ 11.28 (br s, 1H), 7.72-7.46 (m, 1H), 7.34 (m, 2H), 7.27-7.23 (m, 2H), 7.19 (br s, 1H), 6.92-6.88 (m, 2H), 5.32 (br s, 1H), 4.63-3.41 (m, 6H), 2.04 (s, 3H), 1.94-1.70 (m, 4H), 1.52-1.44 (m, 3H).

Synthesis of 7-(((2R)-1-(8-azabicyclo[3.2.1]octan-8-yl)-1-oxopropan-2-yl)oxy)-4-(o-tolyl)isoquinolin-1(2H)-one, Example 86 [Step 4]: To a stirred solution of (2R)-1-(8-azabicyclo[3.2.1]octan-8-yl)-2-((1-chloro-4-(o-tolyl)isoquinolin-7-yl)oxy)propan-1-one (276, 135 mg, 0.3 mmol) in acetic acid (2.7 mL, 46.6 mmol) was added water (0.6 mL, 31.0 mmol). The reaction mixture was heated to reflux for 12 h. The reaction mixture was allowed to cool to ambient temperature and concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford 7-((2R)-1-(8-azabicyclo[3.2.1]octan-8-yl)-(1-oxopropan-2-yl)oxy)-4-(o-tolyl)isoquinolin-1(2H)-one (Example 86, 40 mg). LCMS (ESI) Calcd. for C₂₆H₂₈N₂O₃: 416, found [M+H]⁺=417. ¹H NMR (400 MHz, DMSO-d₆) δ 7.68 (br s, 1H), 7.49-7.47 (d, 1H), 7.34-7.33 (d, 2H), 7.26-7.23 (m, 2H), 7.19-7.18 (m, 1H), 6.90-6.87 (m, 2H), 5.25-5.29 (m, 1H), 4.65-4.38 (m, 2H), 2.06 (s, 3H), 1.80-1.74 (m, 6H), 1.54-1.43 (m, 7H).

### Example 87: Synthesis of (S)-1-((R)-2-((4-(2-chloro-4-fluorophenyl)-1-oxo-1,2-dihydroisoquinolin-7-yl)oxy)propanoyl)piperidine-3-carbonitrile

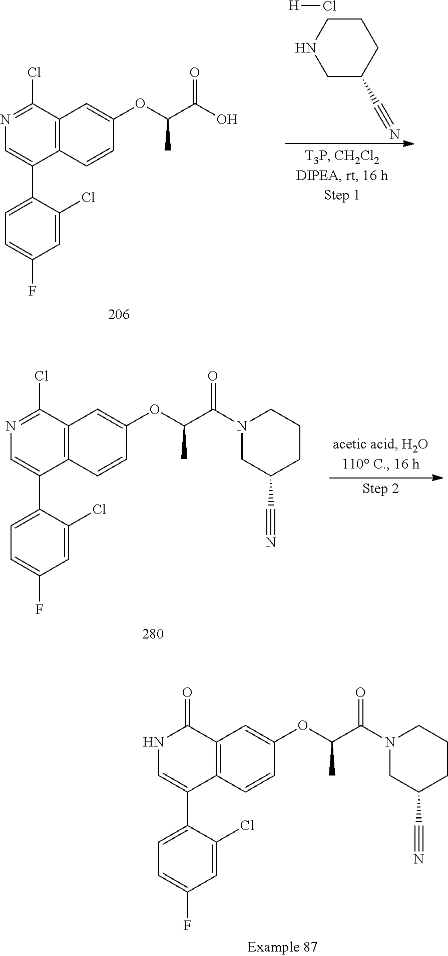

Synthesis of (S)-1-((R)-2-((1-chloro-4-(2-chloro-4-fluorophenyl)isoquinolin-7-yl)oxy)propanoyl)piperidine-3-carbonitrile, 280 [Step 1]: To a stirred solution of (R)-2-((1-chloro-4-(2-chloro-4-fluorophenyl)isoquinolin-7-yl)oxy)propanoic acid (206, 125 mg, 0.3 mmol) in CH₂Cl₂ (4 mL) was added (S)-piperidine-3-carbonitrile hydrochloride (70 mg, 0.5 mmol) and DIPEA (0.2 mL, 1.6 mmol). The reaction mixture was cooled to 0° C., and T₃P (0.3 mL, 0.5 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (S)-1-((R)-2-((1-chloro-4-(2-chloro-4-fluorophenyl)isoquinolin-7-yl)oxy)propanoyl)piperidine-3-carbonitrile (280, 145 mg). LCMS (ESI) Calcd. for C₂₄H₂₀C₁₂FN₃O₂: 471, found [M+H]⁺=472.

Synthesis of (S)-1-((R)-2-((4-(2-chloro-4-fluorophenyl)-1-oxo-1,2-dihydroisoquinolin-7-yl)oxy)propanoyl)piperidine-3-carbonitrile, Example 87 [Step 2]: To a stirred solution of (S)-1-((R)-2-((1-chloro-4-(2-chloro-4-fluorophenyl)isoquinolin-7-yl)oxy)propanoyl)piperidine-3-carbonitrile (280, 145 mg, 0.3 mmol) in acetic acid (2.6 mL, 46.0 mmol) was added water (0.55 mL, 30.7 mmol). The reaction mixture was heated to reflux for 12 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford (S)-1-((R)-2-((4-(2-chloro-4-fluorophenyl)-1-oxo-1,2-dihydroisoquinolin-7-yl)oxy)propanoyl)piperidine-3-carbonitrile (Example 87, 65 mg). LCMS (ESI) Calcd. for C₂₄H₂₁ClFN₃O₃: 453, found [M+H]⁺=454. ¹H NMR (400 MHz, DMSO-d₆) δ 11.41 (br s, 1H), 7.66-7.55 (m, 2H), 7.48-7.45 (m, 1H), 7.34 (m, 1H), 7.26-7.24 (m, 1H), 6.99 (br s, 1H), 6.95-6.93 (m, 1H), 5.58-5.45 (m, 1H), 4.11-3.83 (m, 1H), 3.70 (br s, 2H), 3.52-3.49 (m, 1H), 3.19-3.01 (m, 1H), 2.07 (br s, 1H), 1.88 (br s, 1H), 1.76 (br s, 1H), 1.61 (br s, 1H), 1.46 (br s, 3H).

### Example 88: Synthesis of (R)-2-((1-oxo-4-(o-tolyl)-1,2-dihydroisoquinolin-7-yl)oxy)propanoic acid

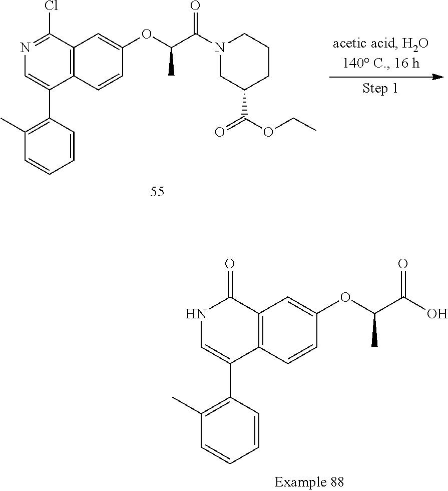

Synthesis of (R)-2-((1-oxo-4-(o-tolyl)-1,2-dihydroisoquinolin-7-yl)oxy)propanoic acid, Example 88 [Step 1]: To a stirred solution of ethyl (S)-1-((R)-2-((1-chloro-4-(o-tolyl)isoquinolin-7-yl)oxy)propanoyl)piperidine-3-carboxylate (55, 170 mg, 0.4 mmol) in acetic acid (3.0 mL, 53.0 mmol) was added water (0.6 mL, 35.3 mmol). The reaction mixture was heated at 140° C. for 16 h. The reaction mixture was concentrated under reduced pressure. The reaction mixture was dissolved in EtOAc, washed with brine, dried over anhydrous Na₂SO₄, filtered, and concentrated under reduced pressure. The product was purified by reverse phase prep-HPLC and lyophilized to afford (R)-2-((1-oxo-4-(o-tolyl)-1,2-dihydroisoquinolin-7-yl)oxy)propanoic acid (Example 88, 80 mg). LCMS (ESI) Calcd. for C₁₉H₁₇NO₄: 323, found [M+H]⁺=324. ¹H NMR (400 MHz, DMSO-d₆) δ 13.14 (br s, 1H), 11.35-11.33 (m, 1H), 7.59-7.58 (m, 1H), 7.35-7.34 (m, 2H), 7.27-7.25 (m, 2H), 7.19-7.18 (m, 1H), 6.92-6.87 (m, 2H), 4.92-4.90 (m, 1H), 2.04-2.03 (s, 3H), 1.54-1.53 (d, 3H).

### Example 89: Synthesis of 2-((4-(2-chloro-4-methylphenyl)-1-oxo-1,2-dihydroisoquinolin-7-yl)oxy)acetonitrile

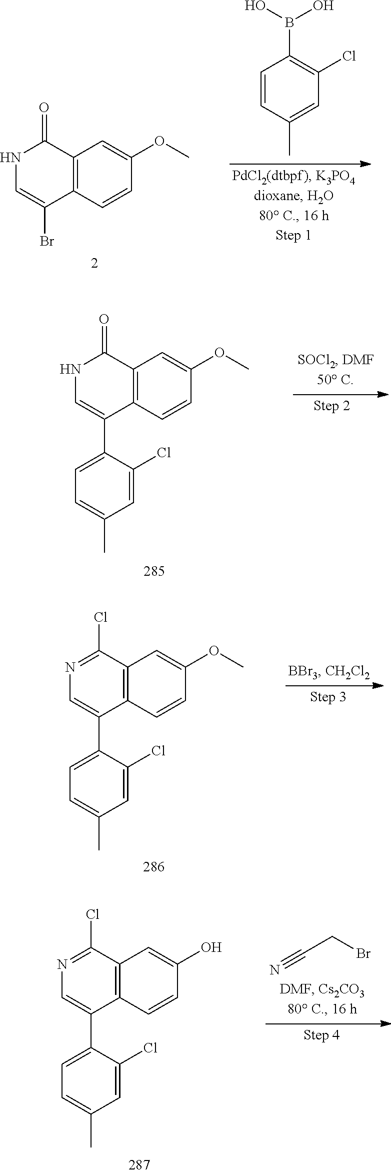

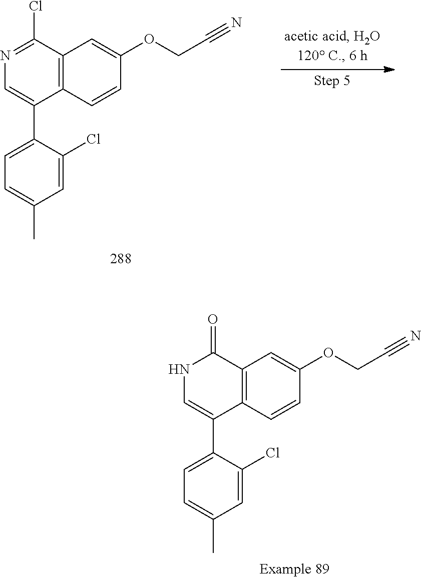 [SMILES: Cc1ccc(-c2cnc(Cl)c3cc(OCC#N)ccc23)c(Cl)c1 ; Cc1ccc(-c2c[nH]c(=O)c3cc(OCC#N)ccc23)c(Cl)c1]

Synthesis of 4-(2-chloro-4-methylphenyl)-7-methoxyisoquinolin-1(2H)-one, 285 [Step 1]: To a stirred solution of 4-bromo-7-methoxyisoquinolin-1(2H)-one (2, 100 mg, 0.4 mmol) in 1,4-dioxane (4 mL) and water (1 mL) was added (2-chloro-4-methyl-phenyl)boronic acid (100 mg, 0.6 mmol) followed by K₃PO₄ (210 mg, 1.0 mmol). The reaction mixture was purged with argon for 5 min., prior to the addition of PdCl₂(dtbpf) (25 mg, 0.04 mmol). The reaction mixture was heated at 100° C. for 16 h. The reaction mixture was filtered through celite. The filtrate was concentrated under reduced pressure. The reaction mixture was dissolved in EtOAc, washed with water, brine, dried over anhydrous Na₂SO₄, filtered, and concentrated under reduced pressure. The product was purified by flash chromatography to afford 4-(2-chloro-4-methylphenyl)-7-methoxyisoquinolin-1(2H)-one (285, 110 mg). LCMS (ESI) Calcd. for C₁₇H₁₄ClNO₂: 299, found [M+H]⁺=300. ¹H NMR (400 MHz, DMSO-d₆) δ 11.39 (br s, 1H), 7.69 (br s, 1H), 7.43 (s, 1H), 7.30-7.25 (m, 3H), 6.97-6.93 (m, 2H), 3.86 (s, 3H), 2.32 (s, 3H).

Synthesis of 1-chloro-4-(2-chloro-4-methylphenyl)-7-methoxyisoquinoline, 286 [Step 2]: To a stirred solution of 4-(2-chloro-4-methylphenyl)-7-methoxyisoquinolin-1(2H)-one (285, 110 mg, 0.4 mmol) in SOCl₂ (1.3 mL, 18.3 mmol) was added DMF (0.03 mL, 0.4 mmol) and the reaction mixture was heated at 50° C. for 16 h. The reaction mixture was concentrated under reduced pressure. The reaction mixture was quenched with ice water and extracted with EtOAc. The organic layer was washed with water, brine, dried over anhydrous Na₂SO₄, filtered, and concentrated under reduced pressure. The product was purified by flash column chromatography to afford 1-chloro-4-(2-chloro-4-methylphenyl)-7-methoxyisoquinoline (286, 115 mg). LCMS (ESI) Calcd. for C₁₇H₁₃Cl₂NO: 317, found [M+H]⁺=318.

Synthesis of 1-chloro-4-(2-chloro-4-methylphenyl)isoquinolin-7-ol, 287 [Step 3]: To a stirred solution 1-chloro-4-(2-chloro-4-methylphenyl)-7-methoxyisoquinoline (286, 120 mg, 0.4 mmol) in CH₂Cl₂ (3 mL) was added dropwise BBr₃ (1.1 mL, 1.1 mmol, 1M in CH₂Cl₂) at 0° C. The reaction mixture was allowed to warm to ambient temperature and stirred for 16 h. The reaction mixture was concentrated under reduced pressure. The reaction mixture was cooled to 0° C., quenched with ice cold water and MeOH, and extracted with EtOAc (3×30 mL). The combined organic layers were washed with brine, dried over anhydrous Na₂SO₄, filtered, and concentrated under reduced pressure. The product was purified by flash column chromatography to afford 1-chloro-4-(2-chloro-4-methylphenyl)isoquinolin-7-ol (287, 114 mg). LCMS (ESI) Calcd. for C₁₆H₁₁Cl₂NO: 303, found [M+H]⁺=304.

Synthesis of 2-((1-chloro-4-(2-chloro-4-methylphenyl)isoquinolin-7-yl)oxy)acetonitrile, 288 [Step 4]: To a stirred solution of 2-bromoacetonitrile (55 mg, 0.5 mmol) and 1-chloro-4-(2-chloro-4-methylphenyl)isoquinolin-7-ol (287, 115 mg, 0.4 mmol) in DMF (2 mL) was added Cs₂CO₃ (365 mg, 1.1 mmol) at ambient temperature. The reaction mixture was gradually heated to 100° C. for 16 h. The reaction mixture was quenched with water and extracted with EtOAc. The organic phase was washed with water, dried over anhydrous Na₂SO₄, filtered, and concentrated under reduced pressure. The product was purified by flash chromatography to afford 2-((1-chloro-4-(2-chloro-4-methylphenyl)isoquinolin-7-yl)oxy)acetonitrile (288, 90 mg). LCMS (ESI) Calcd. for C₁₈H₁₂Cl₂N₂O: 342, found [M+H]⁺=343.

Synthesis of 2-((4-(2-chloro-4-methylphenyl)-1-oxo-1,2-dihydroisoquinolin-7-yl)oxy)acetonitrile, Example 89 [Step 5]: To a stirred solution of 2-((1-chloro-4-(2-chloro-4-methylphenyl)isoquinolin-7-yl)oxy)acetonitrile (288, 200 mg, 0.6 mmol) in acetic acid (5.0 mL, 87.4 mmol) was added water (1.0 mL, 58.3 mmol). The reaction mixture was heated to reflux at 120° C. for 6 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford 2-((4-(2-chloro-4-methylphenyl)-1-oxo-1,2-dihydroisoquinolin-7-yl)oxy)acetonitrile (Example 89, 48 mg). LCMS (ESI) Calcd. for C₁₈H₁₃ClN₂O₂: 324, found [M+H]⁺=325. ¹H NMR (400 MHz, DMSO-d₆) δ 11.51 (br s, 1H), 7.85 (d, 1H), 7.44 (s, 1H), 7.39-7.36 (dd, 1H), 7.32 (d, 1H), 7.27 (d, 1H), 7.03 (br s, 2H), 5.32 (s, 2H), 2.38 (s, 3H).

### Example 90: Synthesis of (R)-2-((4-(2-chloro-4-fluorophenyl)-1-oxo-1,2-dihydroisoquinolin-7-yl)oxy)propanoic acid

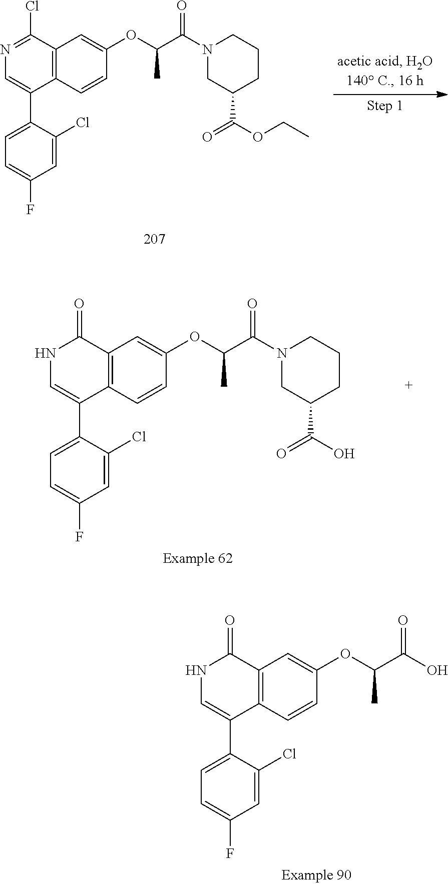 [SMILES: CCOC(=O)[C@H]1CCCN(C(=O)[C@@H](C)Oc2ccc3c(-c4ccc(F)cc4Cl)cnc(Cl)c3c2)C1 ; C[C@@H](Oc1ccc2c(-c3ccc(F)cc3Cl)c[nH]c(=O)c2c1)C(=O)N1CCC[C@H](C(=O)O)C1 ; C[C@@H](Oc1ccc2c(-c3ccc(F)cc3Cl)c[nH]c(=O)c2c1)C(=O)O]

Synthesis of (R)-2-((4-(2-chloro-4-fluorophenyl)-1-oxo-1,2-dihydroisoquinolin-7-yl)oxy)propanoic acid, Example 90 [Step 1]: To a stirred solution of ethyl (S)-1-((R)-2-((1-chloro-4-(2-chloro-4-fluorophenyl)isoquinolin-7-yl)oxy)propanoyl)piperidine-3-carboxylate (207, 150 mg, 0.3 mmol) in acetic acid (2.5 mL, 43.3 mmol) was added water (0.52 mL, 28.9 mmol). The reaction mixture was heated at 140° C. for 16 h. The reaction mixture was concentrated under reduced pressure. The reaction mixture was diluted with EtOAc, washed with water, brine, dried over anhydrous Na₂SO₄, filtered, and concentrated under reduced pressure. The product was purified by reverse phase prep-HPLC and lyophilized to afford (R)-2-((4-(2-chloro-4-fluorophenyl)-1-oxo-1,2-dihydroisoquinolin-7-yl)oxy)propanoic acid (Example 90, 45 mg). LCMS (ESI) Calcd. for C₁₈H₁₃ClFNO₄: 361, found [M+H]⁺=362. ¹H NMR (400 MHz, DMSO-d₆) δ 11.45 (br s, 1H), 7.60-7.56 (m, 2H), 7.47 (br s, 1H), 7.33 (br s, 1H), 7.24-7.22 (m, 1H), 6.95 (s, 1H), 6.91-6.89 (d, 1H), 4.68 (br s, 1H), 1.48 (d, 3H).

### Example 91: Synthesis of (R)-4-(2-chloro-4-fluorophenyl)-7-((1-(4,4-difluoropiperidin-1-yl)-1-oxopropan-2-yl)oxy)isoquinolin-1(2H)-one

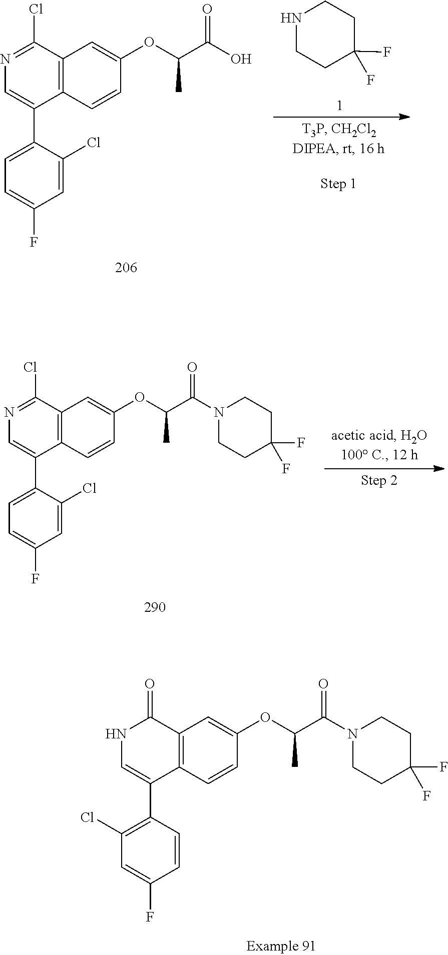 [SMILES: C[C@@H](Oc1ccc2c(-c3ccc(F)cc3Cl)cnc(Cl)c2c1)C(=O)O ; FC1(F)CCNCC1 ; C[C@@H](Oc1ccc2c(-c3ccc(F)cc3Cl)cnc(Cl)c2c1)C(=O)N1CCC(F)(F)CC1 ; C[C@@H](Oc1ccc2c(-c3ccc(F)cc3Cl)c[nH]c(=O)c2c1)C(=O)N1CCC(F)(F)CC1]

Synthesis of (R)-2-((1-chloro-4-(2-chloro-4-fluorophenyl)isoquinolin-7-yl)oxy)-1-(4,4-difluoropiperidin-1-yl)propan-1-one, 290 [Step 1]: To a stirred solution of (R)-2-((1-chloro-4-(2-chloro-4-fluorophenyl)isoquinolin-7-yl)oxy)propanoic acid (206, 120 mg, 0.3 mmol) in CH₂Cl₂ (4 mL) was added 4,4-difluoropiperidine (55 mg, 0.5 mmol) followed by DIPEA (0.21 mL, 1.6 mmol). The reaction mixture was cooled to 0° C., and T₃P (0.3 mL, 0.5 mmol) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (R)-2-((1-chloro-4-(2-chloro-4-fluorophenyl)isoquinolin-7-yl)oxy)-1-(4,4-difluoropiperidin-1-yl)propan-1-one (290, 140 mg). LCMS (ESI) Calcd. for C₂₃H₁₉C₁₂F₃N₂O₂: 482, found [M+H]⁺=483.

Synthesis of (R)-4-(2-chloro-4-fluorophenyl)-7-((1-(4,4-difluoropiperidin-1-yl)-1-oxopropan-2-yl)oxy)isoquinolin-1(2H)-one, Example 91 [Step 2]: To a stirred solution of (R)-2-((1-chloro-4-(2-chloro-4-fluorophenyl)isoquinolin-7-yl)oxy)-1-(4,4-difluoropiperidin-1-yl)propan-1-one (290, 140 mg, 0.3 mmol) in acetic acid (2.5 mL, 43.5 mmol) was added water (0.5 mL, 29.0 mmol). The reaction mixture was heated at 100° C. for 12 h. The reaction mixture was concentrated under reduced pressure. The reaction mixture was diluted with EtOAc, washed with water, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-(4,4-difluoropiperidin-1-yl)-1-oxopropan-2-yl)oxy)isoquinolin-1(2H)-one (Example 91, 85 mg). LCMS (ESI) Calcd. for C₂₃H₂₀ClF₃N₂O₃: 464, found [M+H]⁺=465. ¹H NMR (400 MHz, DMSO-d₆) δ 11.44 (br s, 1H), 7.59-7.56 (m, 2H), 7.49-7.46 (m, 1H), 7.36-7.31 (m, 1H), 7.27-7.25 (d, 1H), 7.00 (s, 1H), 6.95-6.93 (d, 1H), 5.52 (br s, 1H), 3.95 (br s, 2H), 3.53 (br s, 1H), 3.25-3.22 (m, 1H), 2.32 (br s, 1H), 2.07-1.90 (m, 3H), 1.47 (d, 3H).

### Examples 92-93: Synthesis of 7-(((2R)-1-(3-oxa-8-azabicyclo[3.2.1]octan-8-yl)-1-oxopropan-2-yl)oxy)-4-(2-chloro-4-fluorophenyl)isoquinolin-1(2H)-one and 7-(((2R)-1-(8-azabicyclo[3.2.1]octan-8-yl)-1-oxopropan-2-yl)oxy)-4-(2-chloro-4-fluorophenyl)isoquinolin-1(2H)-one

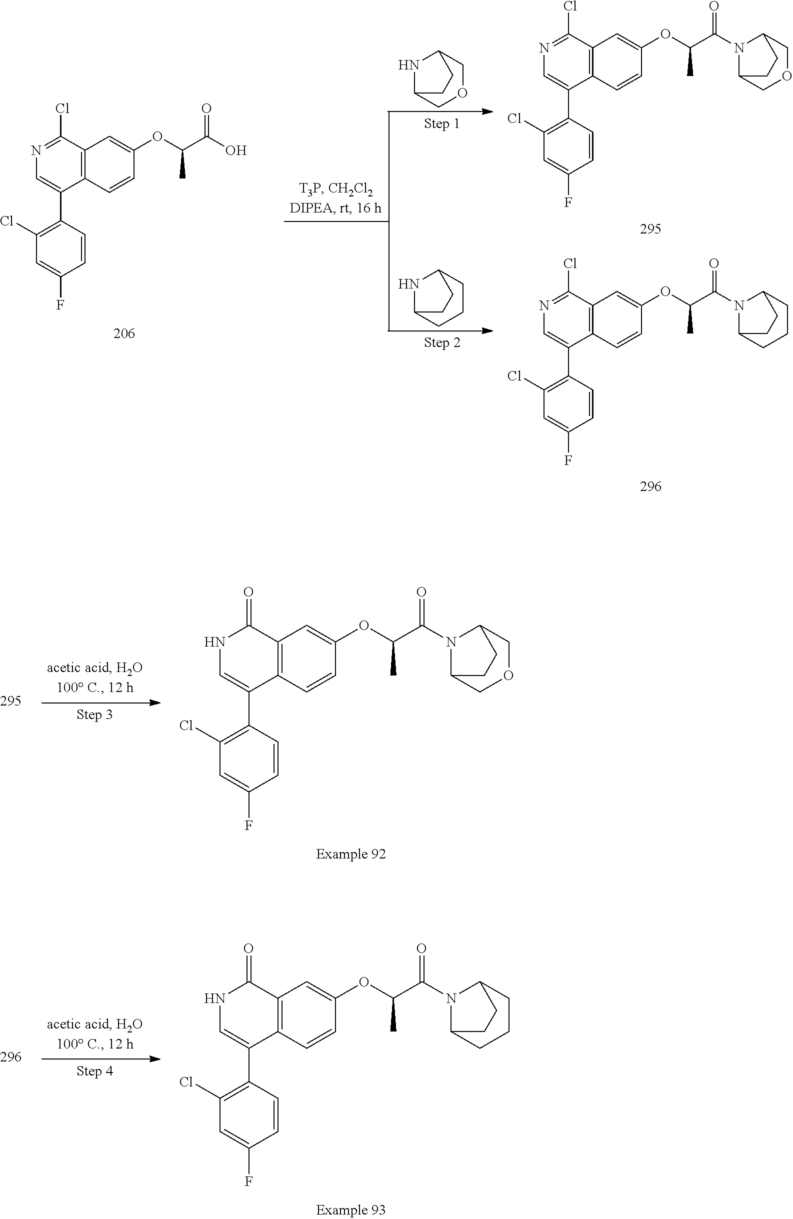

Synthesis of (2R)-1-(3-oxa-8-azabicyclo[3.2.1]octan-8-yl)-2-((1-chloro-4-(2-chloro-4-fluorophenyl)isoquinolin-7-yl)oxy)propan-1-one, 295 [Step 1]: To a stirred solution of 3-oxa-8-azabicyclo[3.2.1]octane (55 mg, 0.5 mmol) in CH₂Cl₂ (4 mL) was added (R)-2-((1-chloro-4-(2-chloro-4-fluorophenyl)isoquinolin-7-yl)oxy)propanoic acid (206, 120 mg, 0.3 mmol) followed by DIPEA (0.2 mL, 1.6 mmol). The reaction mixture was cooled to 0° C., and T₃P (0.3 mL, 0.5 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (2R)-1-(3-oxa-8-azabicyclo[3.2.1]octan-8-yl)-2-((1-chloro-4-(2-chloro-4-fluorophenyl)isoquinolin-7-yl)oxy)propan-1-one (295, 149 mg). LCMS (ESI) Calcd. for C₂₄H₂₁Cl₂FN₂O₃: 474, found [M+H]⁺=475.

Synthesis of (2R)-1-(8-azabicyclo[3.2.1]octan-8-yl)-2-((1-chloro-4-(2-chloro-4-fluorophenyl)isoquinolin-7-yl)oxy)propan-1-one, 296 [Step 2]: To a stirred solution of 8-azabicyclo[3.2.1]octane (55 mg, 0.5 mmol) in CH₂Cl₂ (4 mL) was added (R)-2-((1-chloro-4-(2-chloro-4-fluorophenyl)isoquinolin-7-yl)oxy)propanoic acid (206, 120 mg, 0.3 mmol) and DIPEA (0.2 mL, 1.6 mmol). The reaction mixture was cooled to 0° C., and T₃P (0.3 mL, 0.5 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (2R)-1-(8-azabicyclo[3.2.1]octan-8-yl)-2-((1-chloro-4-(2-chloro-4-fluorophenyl)isoquinolin-7-yl)oxy)propan-1-one (206, 130 mg). LCMS (ESI) Calcd. for C₂₅H₂₃C₁₂FN₂O₂: 472, found [M+H]⁺=473.

Synthesis of 7-(((2R)-1-(3-oxa-8-azabicyclo[3.2.1]octan-8-yl)-1-oxopropan-2-yl)oxy)-4-(2-chloro-4-fluorophenyl)isoquinolin-1(2H)-one, Example 92 [Step 3]: To a stirred solution of (2R)-1-(3-oxa-8-azabicyclo[3.2.1]octan-8-yl)-2-((1-chloro-4-(2-chloro-4-fluorophenyl)isoquinolin-7-yl)oxy)propan-1-one (295, 165 mg, 0.3 mmol) in acetic acid (3.0 mL, 51.8 mmol) was added water (0.6 mL, 34.5 mmol). The reaction mixture was heated at 100° C. for 12 h. The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford 7-(2R)-1-(3-oxa-8-azabicyclo[3.2.1]octan-8-yl)-1-oxopropan-2-yl)oxy)-4-(2-chloro-4-fluorophenyl)isoquinolin-1(2H)-one (Example 92, 50 mg). LCMS (ESI) Calcd. for C₂₄H₂₂ClFN₂O₄: 456, found [M+H]⁺=457. ¹H NMR (400 MHz, DMSO-d₆) δ 11.38 (br s, 1H), 7.72 (m, 1H), 7.61-7.59 (d, 1H), 7.48 (m, 2H), 7.34-7.23 (m, 2H), 7.00-6.92 (m, 2H), 5.31 (m, 1H), 4.63-4.38 (m, 2H), 3.93-3.41 (m, 4H), 1.98-1.72 (m, 3H), 1.50 (dd, 3H).

Synthesis of 7-(((2R)-1-(8-azabicyclo[3.2.1]octan-8-yl)-1-oxopropan-2-yl)oxy)-4-(2-chloro-4-fluorophenyl)isoquinolin-1(2H)-one, Example 93 [Step-4]: To a stirred solution of (2R)-1-(8-azabicyclo[3.2.1]octan-8-yl)-2-((1-chloro-4-(2-chloro-4-fluorophenyl)isoquinolin-7-yl)oxy)propan-1-one (296, 130 mg, 0.3 mmol) in acetic acid (3.9 mL, 68.8 mmol) was added water (0.83 mL, 45.8 mmol). The reaction mixture was heated at 100° C. for 12 h The reaction mixture was concentrated under reduced pressure. The reaction mixture was dissolved in EtOAc, washed with water, brine, dried over anhydrous Na₂SO₄, filtered, and concentrated under reduced pressure. The product was purified by reverse phase prep-HPLC and lyophilized to afford 7-(((2R)-1-(8-azabicyclo[3.2.1]octan-8-yl)-1-oxopropan-2-yl)oxy)-4-(2-chloro-4-fluorophenyl)isoquinolin-1(2H)-one (Example 93, 60 mg). LCMS (ESI) Calcd. for C₂₅H₂₄ClFN₂O₃: 454, found [M+H]⁺=455. ¹H NMR (400 MHz, DMSO-d₆) δ 11.36 (br s, 1H), 7.67 (br s, 1H), 7.61-7.59 (d, 1H), 7.47 (br s, 1H), 7.35 (br s, 1H), 7.25 (br s, 1H), 6.99 (br s, 1H), 6.93 (br s, 1H), 5.25 (br s, 1H), 4.67-4.39 (m, 2H), 3.50-3.45 (m, 1H), 2.07-1.43 (m, 12H).

### Example 94: Synthesis of (S)-1-((R)-2-((1-oxo-4-(o-tolyl)-1,2-dihydroisoquinolin-7-yl)oxy)propanoyl)piperidine-3-carboxamide

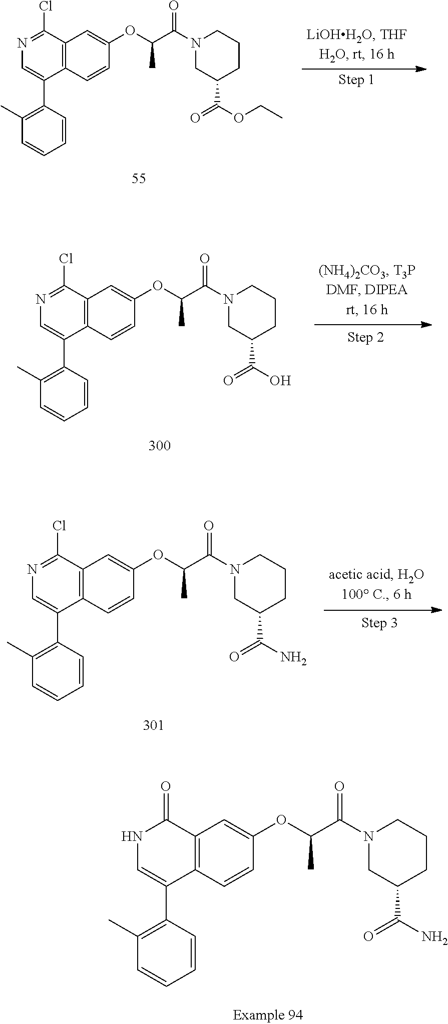

Synthesis of (S)-1-((R)-2-((1-chloro-4-(o-tolyl)isoquinolin-7-yl)oxy)propanoyl)piperidine-3-carboxylic acid, 300 [Step 1]: To a stirred solution of ethyl (S)-1-((R)-2-((1-chloro-4-(o-tolyl)isoquinolin-7-yl)oxy)propanoyl)piperidine-3-carboxylate (55, 70 mg, 0.2 mmol) in THF (4 mL) was added dropwise an aq. solution (2 mL) of LiOH·H₂O (15 mg, 0.4 mmol). The reaction mixture was stirred at ambient temperature for 16 h. The reaction mixture was concentrated under reduced pressure. The reaction mixture was diluted with water, acidified with citric acid, and extracted with EtOAc. The combined organic layers were washed with brine, dried over anhydrous Na₂SO₄, filtered, and concentrated under reduced pressure. The product was purified by reverse phase prep-HPLC and lyophilized to afford the product. The product was further purified via SFC chiral prep-HPLC and lyophilized to afford (S)-1-((R)-2-((1-chloro-4-(o-tolyl)isoquinolin-7-yl)oxy)propanoyl)piperidine-3-carboxylic acid (300, 20 mg). LCMS (ESI) Calcd. for C₂₅H₂₅ClN₂O₄: 453, found [M+H]⁺=453. ¹H NMR (400 MHz, DMSO-d₆) δ 8.04 (br s, 1H), 7.50-7.42 (m, 4H), 7.36-7.34 (m, 2H), 7.25 (br s, 2H), 5.61 (br s, 1H), 4.44-3.85 (m, 3H), 2.06 (br s, 1H), 1.97 (s, 3H), 1.82-1.61 (m, 2H), 1.51 (d, 3H), 1.23 (br s, 3H).

Chiral SFC: Chiral separation was performed on a Thar SFC-80 series instrument. Column was a I-Cellulose C (21×250 mm), 5 μm, operating at 35° C. temperature with flow rate of 60 gm/min. Mobile phase: 60% CO₂ in super critical state and 40% MeOH, held isocratic for up to 8 min. at 100 bar with detection at 228 nm wavelength.

Synthesis of (S)-1-((R)-2-((1-chloro-4-(o-tolyl)isoquinolin-7-yl)oxy)propanoyl)piperidine-3-carboxamide, 301 [Step 2]: To a stirred solution of (S)-1-((R)-2-((1-chloro-4-(o-tolyl)isoquinolin-7-yl)oxy)propanoyl)piperidine-3-carboxylic acid (300, 300 mg, 0.6 mmol) in DMF (4 mL) was added DIPEA (0.6 mL, 3.3 mmol), (NH₄)₂CO₃ (320 mg, 3.3 mmol), and T₃P (0.6 mL, 1.0 mmol, 50% EtOAc) at 0° C. The reaction mixture was allowed to warm to ambient temperature and stirred for 16 h. The reaction mixture was quenched with ice cold water and extracted with EtOAc. The organic phase was washed with brine, dried over anhydrous Na₂SO₄, filtered, and concentrated under reduced pressure. The product was purified by reverse phase prep-HPLC and lyophilized to afford (5)-1-((R)-2-((1-chloro-4-(o-tolyl)isoquinolin-7-yl)oxy)propanoyl)piperidine-3-carboxamide (301, 260 mg). LCMS (ESI) Calcd. for C₂₅H₂₆C₁N₃O₃: 452, found [M+H]⁺=452. ¹H NMR (400 MHz, DMSO-d₆) δ 8.04-8.03 (m, 1H), 7.50-7.48 (m, 1H), 7.42-7.31 (m, 5H), 7.29-7.24 (m, 1H), 6.97 (br s, 1H), 6.82 (br s, 1H), 5.64-5.63 (m, 1H), 4.41-4.38 (m, 1H), 4.08 (br s, 1H), 3.08-2.61 (m, 3H), 2.32-2.24 (m, 1H), 2.32-2.24 (m, 1H), 1.97 (s, 3H), 1.82-1.53 (m, 2H), 1.50-1.49 (d, 3H).

Synthesis of (S)-1-((R)-2-((1-oxo-4-(o-tolyl)-1,2-dihydroisoquinolin-7-yl)oxy)propanoyl)piperidine-3-carboxamide, Example 94 [Step 3]: To a stirred solution of (S)-1-((R)-2-((1-chloro-4-(o-tolyl)isoquinolin-7-yl)oxy)propanoyl)piperidine-3-carboxamide (301, 180 mg, 0.4 mmol) in acetic acid (3.4 mL, 60 mmol) was added water (0.8 mL, 43.8 mmol). The reaction mixture was stirred at 100° C. for 6 h. The reaction mixture was concentrated under reduced pressure. The reaction mixture was quenched with ice cold water and extracted with EtOAc. The organic phase was washed with water, brine, dried over anhydrous Na₂SO₄, filtered, and concentrated under reduced pressure. The product was purified by reverse phase prep-HPLC and lyophilized to afford (S)-1-((R)-2-((1-oxo-4-(o-tolyl)-1,2-dihydroisoquinolin-7-yl)oxy)propanoyl)piperidine-3-carboxamide (Example 94, 50 mg). LCMS (ESI) Calcd. for C₂₅H₂₇N₃O₄: 434, found [M+H]⁺=434. ¹H NMR (400 MHz, DMSO-d₆) δ 11.33-11.28 (m, 1H), 7.61-7.58 (m, 1H), 7.41-7.34 (m, 3H), 7.28-7.18 (m, 3H), 6.98-6.83 (m, 3H), 5.48-5.45 (m, 1H), 4.39-4.36 (m, 1H), 4.09-4.03 (m, 2H), 3.05-2.55 (m, 2H), 2.32-2.22 (m, 1H), 2.07-2.03 (s, 3H), 1.97-1.59 (m, 3H), 1.46-1.42 (d, 3H).

### Examples 95-96: Synthesis of chiral 4-(2-chloro-4-fluorophenyl)-7-(((2R)-1-(3-(methylsulfonyl)piperidin-1-yl)-1-oxopropan-2-yl)oxy)isoquinolin-1(2H)-one

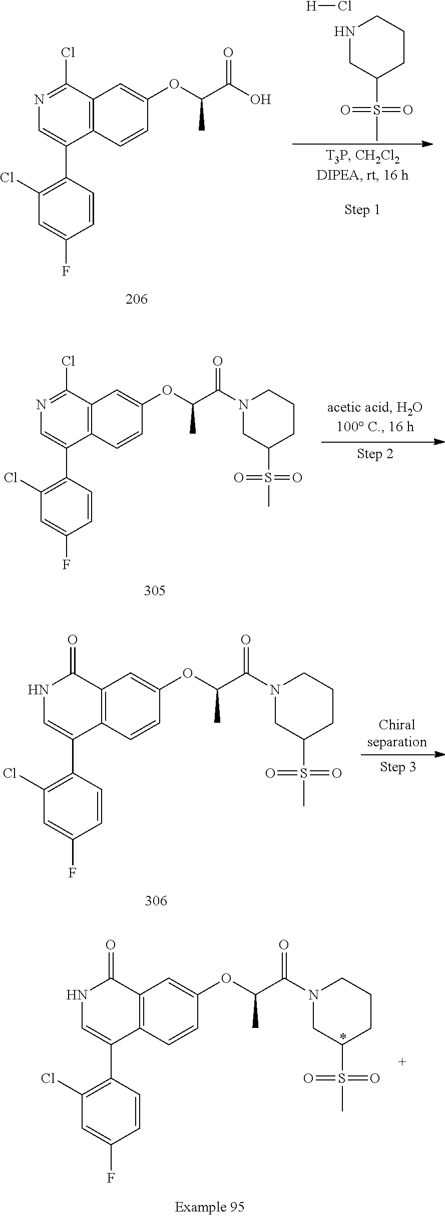

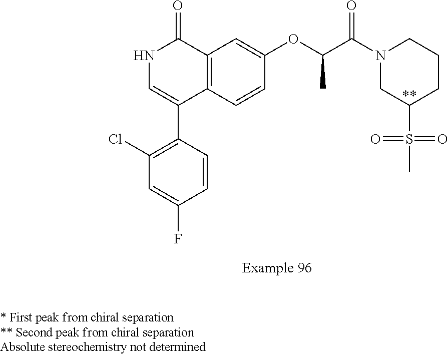 [SMILES: C[C@@H](Oc1ccc2c(-c3ccc(F)cc3Cl)c[nH]c(=O)c2c1)C(=O)N1CCCC(S(C)(=O)=O)C1]

Synthesis of (2R)-2-((1-chloro-4-(2-chloro-4-fluorophenyl)isoquinolin-7-yl)oxy)-1-(3-(methylsulfonyl)piperidin-1-yl)propan-1-one, 305 [Step 1]: To a stirred solution of (R)-2-((1-chloro-4-(2-chloro-4-fluorophenyl)isoquinolin-7-yl)oxy)propanoic acid (206, 250 mg, 0.7 mmol) in CH₂Cl₂ (5 mL) was added 3-methylsulfonylpiperidine hydrochloride (195 mg, 1.0 mmol) and DIPEA (0.6 mL, 3.3 mmol). The reaction mixture was cooled to 0° C., and T₃P (0.59 mL, 1.0 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (2R)-2-((1-chloro-4-(2-chloro-4-fluorophenyl)isoquinolin-7-yl)oxy)-1-(3-(methylsulfonyl)piperidin-1-yl)propan-1-one (305, 200 mg). LCMS (ESI) Calcd. for C₂₄H₂₃Cl₂FN₂O₄S: 524, found [M+H]⁺=525. ¹H NMR (400 MHz, DMSO-d₆) δ 8.09 (br s, 1H), 7.71 (br s, 1H), 7.56-7.40 (m, 5H), 5.65 (br s, 1H), 4.68 (br s, 1H), 4.03 (br s, 2H), 2.99-2.86 (m, 4H), 2.24-1.75 (m, 5H), 1.52 (br s, 3H).

Synthesis of 4-(2-chloro-4-fluorophenyl)-7-(((2R)-1-(3-(methylsulfonyl)piperidin-1-yl)-1-oxopropan-2-yl)oxy)isoquinolin-1(2H)-one, 306 [Step 2]: To a stirred solution of (2R)-2-((1-chloro-4-(2-chloro-4-fluorophenyl)isoquinolin-7-yl)oxy)-1-(3-(methylsulfonyl)piperidin-1-yl)propan-1-one (305, 200 mg, 0.4 mmol) in acetic acid (3.3 mL, 57.1 mmol) was added water (0.7 mL, 38.1 mmol). The reaction mixture was heated at 100° C. for 16 h. The reaction mixture was concentrated under reduced pressure. The reaction mixture was diluted with EtOAc, washed with water, brine, dried over anhydrous Na₂SO₄, filtered, and concentrated under reduced pressure. The product was purified by reverse phase prep-HPLC and lyophilized to afford 4-(2-chloro-4-fluorophenyl)-7-(((2R)-1-(3-(methylsulfonyl)piperidin-1-yl)-1-oxopropan-2-yl)oxy)isoquinolin-1(2H)-one (306, 100 mg). LCMS (ESI) Calcd. for C₂₄H₂₄ClFN₂O₅S: 506, found [M+H]⁺=507.

Synthesis of chiral 4-(2-chloro-4-fluorophenyl)-7-(((2R)-1-(3-(methylsulfonyl)piperidin-1-yl)-1-oxopropan-2-yl)oxy)isoquinolin-1(2H)-one, Example 95 and Example 96 [Step 3]: 4-(2-chloro-4-fluorophenyl)-7-(((2R)-1-(3-(methylsulfonyl)piperidin-1-yl)-1-oxopropan-2-yl)oxy)isoquinolin-1(2H)-one (306, 80 mg) was purified by normal phase chiral prep-HPLC and lyophilized. The first product was isolated as 4-(2-chloro-4-fluorophenyl)-7-(((2R)-1-(3-(methylsulfonyl)piperidin-1-yl)-1-oxopropan-2-yl)oxy)isoquinolin-1(2H)-one, Peak 1 (Example 95, 38 mg) and the second product as 4-(2-chloro-4-fluorophenyl)-7-(((2R)-1-(3-(methylsulfonyl)piperidin-1-yl)-1-oxopropan-2-yl)oxy)isoquinolin-1(2H)-one, Peak 2 (Example 96, 32 mg). The absolute stereochemistry of these Examples was not determined.

Example 95: 4-(2-chloro-4-fluorophenyl)-7-(((2R)-1-(3-(methylsulfonyl)piperidin-1-yl)-1-oxopropan-2-yl)oxy)isoquinolin-1(2H)-one, Peak 1: LCMS (ESI) Calcd. for C₂₄H₂₄ClFN₂O₅S: 506, found [M+H]⁺=507. ¹H NMR (400 MHz, DMSO-d₆) δ 11.49 (br s, 1H), 7.62-7.55 (m, 2H), 7.47 (br s, 1H), 7.36-7.24 (m, 2H), 7.02-6.93 (m, 2H), 5.50 (br s, 1H), 4.70-4.07 (m, 2H), 3.16 (br s, 2H), 2.96 (s, 3H), 2.86 (br s, 1H), 2.32-1.39 (m, 7H).

Example 96: 4-(2-chloro-4-fluorophenyl)-7-(((2R)-1-(3-(methylsulfonyl)piperidin-1-yl)-1-oxopropan-2-yl)oxy)isoquinolin-1(2H)-one, Peak 2: LCMS (ESI) Calcd. for C₂₄H₂₄ClFN₂O₅S: 506, found [M+H]⁺=507. ¹H NMR (400 MHz, DMSO-d₆) δ 11.44 (br s, 1H), 7.61 (br s, 1H), 7.55 (br s, 1H), 7.49-7.45 (m, 1H), 7.36-7.32 (m, 1H), 7.27-7.23 (m, 1H), 7.00 (d, 1H), 6.95 (d, 1H), 5.46 (br s, 1H), 4.70-4.07 (m, 2H), 3.16 (m, 2H), 2.98 (s, 3H), 2.86 (m, 1H), 2.27-1.46 (m, 7H).

Chiral prep-HPLC: Chiral separation was performed on an Agilent 1200 series instrument. Column was a Chiralpak IC (250×21 mm), 5 μm, operating at ambient temperature with flow rate of 21.0 mL/min. Mobile phase: 60% hexane, 20% CH₂Cl₂, and 20% ethanol, held isocratic for up to 27 min. with detection at 282 nm wavelength.

Examples 97-98: Synthesis of chiral 7-(((2R)-1-(3-(methylsulfonyl)piperidin-1-yl)-1-oxopropan-2-yl)oxy)-4-(o-tolyl)isoquinolin-1(2H)-one.

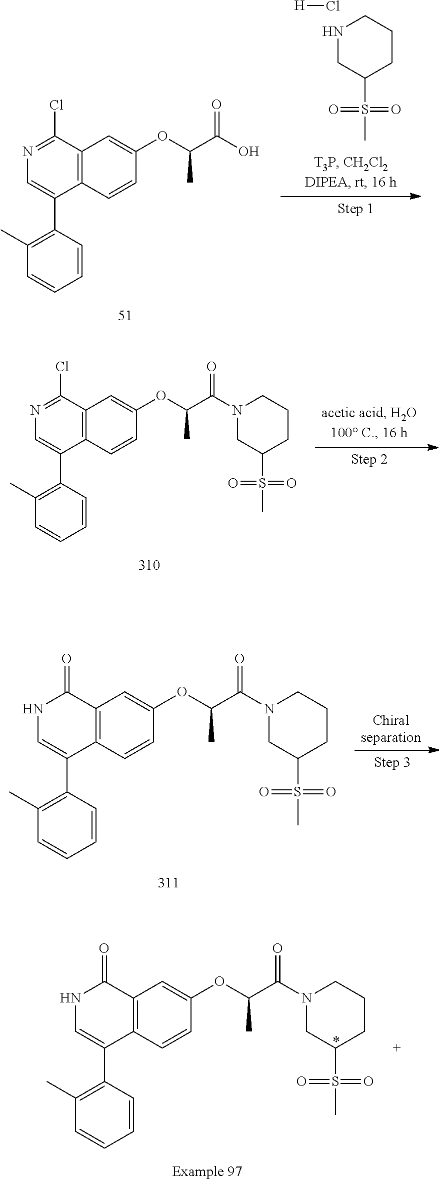

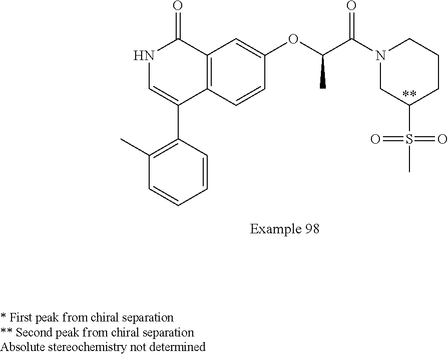 [SMILES: Cc1ccccc1-c1c[nH]c(=O)c2cc(O[C@H](C)C(=O)N3CCCC(S(C)(=O)=O)C3)ccc12]

Synthesis of (2R)-2-((1-chloro-4-(o-tolyl)isoquinolin-7-yl)oxy)-1-(3-(methylsulfonyl)piperidin-1-yl)propan-1-one, 310 [Step 1]: To a stirred solution of (R)-2-((1-chloro-4-(o-tolyl)isoquinolin-7-yl)oxy)propanoic acid (51, 220 mg, 0.6 mmol) in CH₂Cl₂ (5 mL) was added 3-methylsulfonylpiperidine hydrochloride (195 mg, 1.0 mmol) followed by DIPEA (0.6 mL, 3.2 mmol). The reaction mixture was cooled to 0° C., and T₃P (0.6 mL, 1.0 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure to afford (2R)-2-((1-chloro-4-(o-tolyl)isoquinolin-7-yl)oxy)-1-(3-(methylsulfonyl)piperidin-1-yl)propan-1-one (310, 280 mg). LCMS (ESI) Calcd. for C₂₅H₂₇C₁N₂O₄S: 486, found [M+H]⁺=487.

Synthesis of 7-(((2R)-1-(3-(methylsulfonyl)piperidin-1-yl)-1-oxopropan-2-yl)oxy)-4-(o-tolyl)isoquinolin-1(2H)-one, 311 [Step 2]: To a stirred solution of (2R)-2-((1-chloro-4-(o-tolyl)isoquinolin-7-yl)oxy)-1-(3-(methylsulfonyl)piperidin-1-yl)propan-1-one (310, 280 mg, 0.6 mmol) in acetic acid (4.9 mL, 86.2 mmol) was added water (1.0 mL, 57.5 mmol). The reaction mixture was heated at 100° C. for 16 h. The reaction mixture was concentrated under reduced pressure. The reaction mixture was diluted with EtOAc, washed with water, brine, dried over anhydrous Na₂SO₄, filtered, and concentrated under reduced pressure. The product was purified by reverse phase prep-HPLC and lyophilized to afford 7-(((2R)-1-(3-(methylsulfonyl)piperidin-1-yl)-1-oxopropan-2-yl)oxy)-4-(o-tolyl)isoquinolin-1(2H)-one (311, 100 mg). LCMS (ESI) Calcd. for C₂₅H₂₈N₂O₅S: 468, found [M+H]⁺=469.

Synthesis of chiral 7-(((2R)-1-(3-(methylsulfonyl)piperidin-1-yl)-1-oxopropan-2-yl)oxy)-4-(o-tolyl)isoquinolin-1(2H)-one, Example 97 and Example 98 [Step 3]: 7-(((2R)-1-(3-(methylsulfonyl)piperidin-1-yl)-1-oxopropan-2-yl)oxy)-4-(o-tolyl)isoquinolin-1(2H)-one (311, 100 mg, 0.2 mmol) was purified by normal phase chiral prep-HPLC and lyophilized. The first product was isolated as 7-(((2R)-1-(3-(methylsulfonyl)piperidin-1-yl)-1-oxopropan-2-yl)oxy)-4-(o-tolyl)isoquinolin-1(2H)-one, Peak 1 (Example 97, 48 mg) and the second as 7-(((2R)-1-(3-(methylsulfonyl)piperidin-1-yl)-1-oxopropan-2-yl)oxy)-4-(o-tolyl)isoquinolin-1(2H)-one, Peak 2 (Example 98, 38 mg). The absolute stereochemistry of these Examples was not determined.

Example 97: 7-(((2R)-1-(3-(methylsulfonyl)piperidin-1-yl)-1-oxopropan-2-yl)oxy)-4-(o-tolyl)isoquinolin-1(2H)-one, Peak 1: LCMS (ESI) Calcd. for C₂₅H₂₈N₂O₅S: 468, found [M+H]⁺=469. ¹H NMR (400 MHz, DMSO-d₆) δ 11.33 (br s, 1H), 7.60-7.55 (m, 1H), 7.35-7.17 (m, 5H), 6.91 (br s, 2H), 5.45 (br s, 1H), 4.66-4.05 (m, 2H), 3.73-3.05 (m, 3H), 2.96 (s, 3H), 2.86 (br s, 1H), 2.32-1.39 (m, 1H), 2.03 (s, 3H), 1.86-1.73 (m, 2H), 1.47 (br s, 3H).

Example 98: 7-(((2R)-1-(3-(methylsulfonyl)piperidin-1-yl)-1-oxopropan-2-yl)oxy)-4-(o-tolyl)isoquinolin-1(2H)-one, Peak 2: LCMS (ESI) Calcd. for C₂₅H₂₈N₂O₅S: 468, found [M+H]⁺=469. ¹H NMR (400 MHz, DMSO-d₆) δ 11.33 (br s, 1H), 7.56 (br s, 1H), 7.35 (br s, 2H), 7.28-7.17 (m, 3H), 6.89 (br s, 2H), 5.44 (br s, 1H), 4.71-4.06 (m, 2H), 3.18 (br s, 2H), 2.98 (s, 3H), 2.86 (br s, 1H), 2.32-2.24 (m, 1H), 2.03 (s, 3H), 1.88-1.80 (m, 3H), 1.46 (br s, 3H).

Chiral prep-HPLC: Chiral separation was performed on an Agilent 1200 series instrument. Column was a Chiralpak IC (250×21 mm), 5 μm, operating at ambient temperature with flow rate of 21.0 mL/min. Mobile phase: 60% Hexane, 20% CH₂Cl₂, and 20% ethanol, held isocratic for up to 30 min. with detection at 284 nm wavelength.

### Example 99: Synthesis of 2-((4-(2,6-dimethylphenyl)-1-oxo-1,2-dihydroisoquinolin-7-yl)oxy)acetonitrile

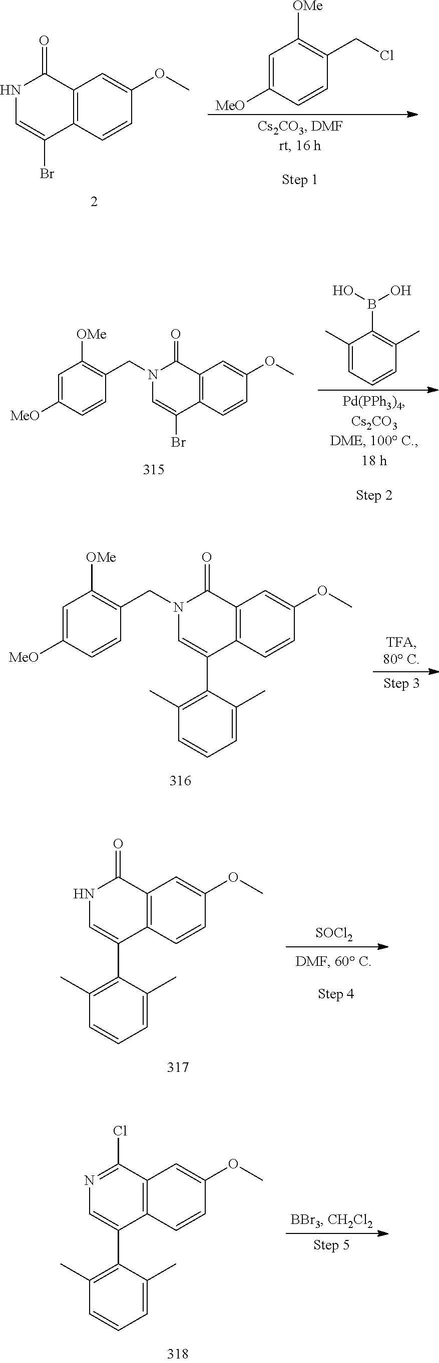

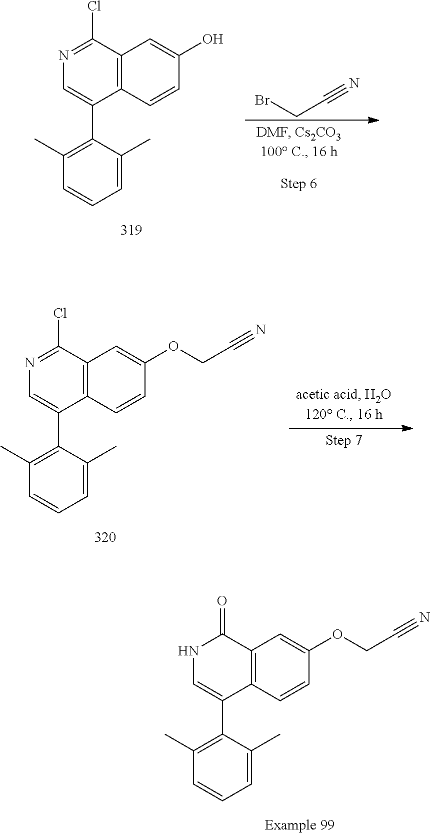

Synthesis of 4-bromo-2-(2,4-dimethoxybenzyl)-7-methoxyisoquinolin-1(2H)-one, 315 [Step 1]: To a stirred solution of 4-bromo-7-methoxyisoquinolin-1(2H)-one (2, 950 mg, 3.7 mmol) in DMF (10 mL) was added Cs₂CO₃ (2.4 g, 7.5 mmol). The reaction mixture was cooled to 0° C. A freshly prepared solution of 1-(chloromethyl)-2,4-dimethoxy-benzene (1.0 g, 5.6 mmol) in diethyl ether was added dropwise to the reaction mixture. The reaction mixture was stirred at ambient temperature for 16 h. The reaction mixture was diluted with water and extracted with EtOAc. The organic layer was dried over anhydrous Na₂SO₄, filtered, and concentrated under reduced pressure. The product was purified by column chromatography to afford 4-bromo-2-(2,4-dimethoxybenzyl)-7-methoxyisoquinolin-1(2H)-one (315, 800 mg). LCMS (ESI): Calcd. for C₁₉H₁₈BrNO₄: 403, found [M+H]⁺=404. ¹H NMR (400 MHz, CDCl₃) δ 7.86 (s, 1H), 7.70 (d, 1H), 7.42 (s, 1H), 7.36 (d, 1H), 7.31-7.25 (m, 1H), 6.45 (s, 2H), 5.10 (s, 2H), 3.92 (s, 3H), 3.85 (s, 3H), 3.78 (s, 3H).

Synthesis of 2-(2,4-dimethoxybenzyl)-4-(2,6-dimethylphenyl)-7-methoxyisoquinolin-1(2H)-one, 316 [Step 2]: To a sealed tube was added 4-bromo-2-(2,4-dimethoxybenzyl)-7-methoxyisoquinolin-1(2H)-one (315, 200 mg, 0.5 mmol), (2,6-dimethylphenyl)boronic acid (120 mg, 0.8 mmol), and Cs₂CO₃ (320 mg, 0.9 mmol). The reaction mixture was purged with nitrogen for 5 min., prior to the addition of 1,2-dimethoxyethane (6 mL) and tetrakis(triphenylphosphine)palladium(0) (55 mg, 0.05 mmol). The reaction mixture was stirred at 100° C. for 18 h. The reaction mixture was diluted with EtOAc, washed with brine, dried over anhydrous Na₂SO₄, filtered, and concentrated under reduced pressure. The product was purified by column chromatography to afford 2-(2,4-dimethoxybenzyl)-4-(2,6-dimethylphenyl)-7-methoxyisoquinolin-1(2H)-one (316, 160 mg). LCMS (ESI) Calcd. for C₂₇H₂₇NO₄: 429, found [M+H]⁺=430.

Synthesis of 4-(2,6-dimethylphenyl)-7-methoxyisoquinolin-1(2H)-one, 317 [Step 3]: To a sealed tube was added 2-(2,4-dimethoxybenzyl)-4-(2,6-dimethylphenyl)-7-methoxyisoquinolin-1(2H)-one (316, 550 mg, 1.3 mmol) and trifluoroacetic acid (9.8 mL, 128 mmol). The reaction mixture was heated at 80° C. for 16 h. The reaction mixture was concentrated under reduced pressure. The reaction mixture was diluted with EtOAc, washed with brine, dried over anhydrous Na₂SO₄, filtered, and concentrated under reduced pressure. The product was purified by column chromatography to afford 4-(2,6-dimethylphenyl)-7-methoxyisoquinolin-1(2H)-one (317, 300 mg). LCMS (ESI) Calcd. for C₁₈H₁₇NO₂: 279, found [M+H]⁺=280. ¹H NMR (400 MHz, DMSO-d₆) δ 11.36 (s, 1H), 7.71 (s, 1H), 7.26-7.16 (m, 4H), 6.83-6.75 (m, 2H), 3.86 (s, 3H), 1.96 (s, 6H).

Synthesis of 1-chloro-4-(2,6-dimethylphenyl)-7-methoxyisoquinoline, 318 [Step 4]: To a stirred solution of 4-(2,6-dimethylphenyl)-7-methoxyisoquinolin-1(2H)-one (317, 250 mg, 0.9 mmol) in SOCl₂ (6.5 mL, 89.5 mmol) was added DMF (0.1 mL, 0.9 mmol) at ambient temperature. The reaction mixture was heated at 60° C. for 18 h. The reaction mixture was concentrated under reduced pressure. The reaction mixture was cooled to 0° C., quenched with ice cold water, and extracted with EtOAc. The combined organic layers were washed with brine, dried over anhydrous Na₂SO₄, filtered, and concentrated under reduced pressure. The product was purified by column chromatography to afford 1-chloro-4-(2,6-dimethylphenyl)-7-methoxyisoquinoline (318, 210 mg). LCMS (ESI) Calcd. for C₁₈H₁₆ClNO: 297, found [M+H]⁺=298.

Synthesis of 1-chloro-4-(2,6-dimethylphenyl)isoquinolin-7-ol, 319 [Step 5]: To a stirred solution of 1-chloro-4-(2,6-dimethylphenyl)-7-methoxyisoquinoline (318, 200 mg, 0.7 mmol) in CH₂Cl₂ (5 mL) was added dropwise BBr₃ (505 mg, 2.0 mmol, 1 M in CH₂Cl₂) at 0° C. The reaction mixture was allowed to warm to ambient temperature and stirred for 16 h. The reaction mixture was concentrated under reduced pressure. The reaction mixture was cooled to 0° C., quenched with MeOH, and concentrated under reduced pressure. The reaction mixture was dissolved in EtOAc, washed with brine, dried over anhydrous Na₂SO₄, filtered, and concentrated under reduced pressure to afford 1-chloro-4-(2,6-dimethylphenyl)isoquinolin-7-ol (319, 180 mg). LCMS (ESI) Calcd. for C₁₇H₁₄ClNO: 283, found [M+H]⁺=284.

Synthesis of 2-((1-chloro-4-(2,6-dimethylphenyl)isoquinolin-7-yl)oxy)acetonitrile, 320 [Step 6]: To a stirred solution of 1-chloro-4-(2,6-dimethylphenyl)isoquinolin-7-ol (319, 180 mg, 0.6 mmol) and 2-bromoacetonitrile (115 mg, 0.9 mmol) in DMF (4 mL) was added Cs₂CO₃ (620 mg, 1.9 mmol). The reaction mixture was stirred at 100° C. for 16 h. The reaction mixture was quenched with water and extracted with EtOAc. The organic layer was dried over anhydrous Na₂SO₄, filtered, and concentrated under reduced pressure. The product was purified by flash chromatography to afford 2-((1-chloro-4-(2,6-dimethylphenyl)isoquinolin-7-yl)oxy)acetonitrile (320, 170 mg). LCMS (ESI) Calcd. for C₁₉H₁₅ClN₂O: 322, found [M+H]⁺=323.

Synthesis of 2-((4-(2,6-dimethylphenyl)-1-oxo-1,2-dihydroisoquinolin-7-yl)oxy)acetonitrile, Example 99 [Step 7]: To a stirred solution of 2-((1-chloro-4-(2,6-dimethylphenyl)isoquinolin-7-yl)oxy)acetonitrile (320, 170 mg, 0.5 mmol) in acetic acid (4.5 mL, 79.0 mmol) was added water (0.9 mL, 52.7 mmol). The reaction mixture was heated to reflux at 120° C. for 16 h. The reaction mixture was quenched with water and extracted with EtOAc. The organic layer was dried over anhydrous Na₂SO₄, filtered, and concentrated under reduced pressure. The product was purified by reverse phase prep-HPLC and lyophilized to afford 2-((4-(2,6-dimethylphenyl)-1-oxo-1,2-dihydroisoquinolin-7-yl)oxy)acetonitrile (Example 99, 75 mg). LCMS (ESI) Calcd. for C₁₉H₁₆N₂O₂: 304, found [M+H]⁺=305. ¹H NMR (400 MHz, DMSO-d₆) δ 11.50 (br s, 1H), 7.87 (s, 1H), 7.34 (d, 1H), 7.27-7.23 (m, 1H), 7.19-7.17 (m, 2H), 6.91 (s, 1H), 6.82 (d, 1H), 5.32 (s, 2H), 1.97 (s, 6H).

### Example 100: Synthesis of 4-(2-chloro-4-fluorophenyl)-7-(((R)-1-((2S,6R)-2,6-dimethylmorpholino)-1-oxopropan-2-yl)oxy)isoquinolin-1(2H)-one

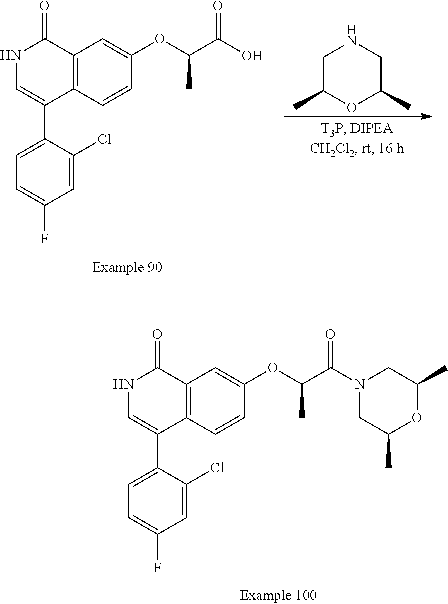

Synthesis of 4-(2-chloro-4-fluorophenyl)-7-(((R)-1-((2S,6R)-2,6-dimethylmorpholino)-1-oxopropan-2-yl)oxy)isoquinolin-1(2H)-one, Example 100: To a stirred solution of (R)-2-((4-(2-chloro-4-fluorophenyl)-1-oxo-1,2-dihydroisoquinolin-7-yl)oxy)propanoic acid (Example 90, 30 mg, 0.1 mmol) in CH₂Cl₂ (10 mL) was added DIPEA (0.1 mL, 0.4 mmol) followed by (2S,6R)-2,6-dimethylmorpholine (15 mg, 0.1 mmol). The reaction mixture was cooled to 0° C., and T₃P (0.1 mL, 0.1 mmol, 50% in EtOAc) was added. The reaction mixture was allowed to warm to ambient temperature and stirred for 16 h. The reaction mixture was quenched with ice cold water and extracted with CH₂Cl₂ (×2). The combined organic layers were washed with brine, dried over anhydrous Na₂SO₄, filtered, and concentrated under reduced pressure. The product was purified by reverse phase prep-HPLC and lyophilized to afford 4-(2-chloro-4-fluorophenyl)-7-(((R)-1-((2S,6R)-2,6-dimethylmorpholino)-1-oxopropan-2-yl)oxy)isoquinolin-1(2H)-one (Example 100, 28 mg). LCMS (ESI) Calcd. for C₂₄H₂₄ClFN₂O₄: 458, found [M+H]⁺=459. ¹H NMR (400 MHz, DMSO-d₆) (at 100° C.) δ 11.12 (br s, 1H), 7.67 (s, 1H), 7.53-7.46 (m, 2H), 7.30-7.24 (m, 2H), 6.97 (br s, 2H), 5.38 (br s, 1H), 4.16 (br s, 2H), 3.74 (br s, 2H), 3.46-3.42 (m, 2H), 1.50 (s, 3H), 1.10 (s, 6H).

### Example 101: Synthesis of (R)-4-(2-chloro-4-fluorophenyl)-7-((1-(1,1-dioxidothiomorpholino)-1-oxopropan-2-yl)oxy)isoquinolin-1(2H)-one

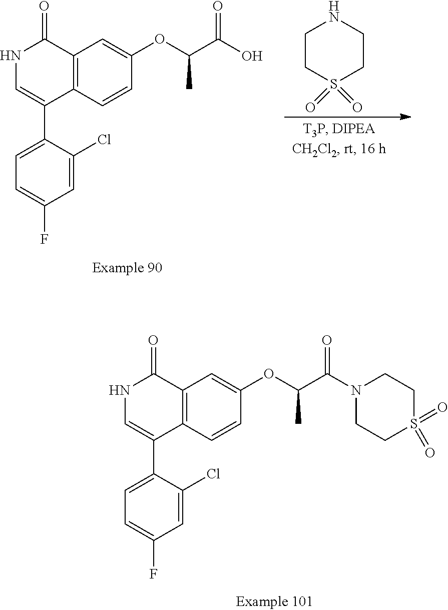 [SMILES: C[C@@H](Oc1ccc2c(-c3ccc(F)cc3Cl)c[nH]c(=O)c2c1)C(=O)O ; O=S1(=O)CCNCC1 ; C[C@@H](Oc1ccc2c(-c3ccc(F)cc3Cl)c[nH]c(=O)c2c1)C(=O)N1CCS(=O)(=O)CC1]

Synthesis of (R)-4-(2-chloro-4-fluorophenyl)-7-((1-(1,1-dioxidothiomorpholino)-1-oxopropan-2-yl)oxy)isoquinolin-1(2H)-one, Example 101: To a stirred solution of (R)-2-((4-(2-chloro-4-fluorophenyl)-1-oxo-1,2-dihydroisoquinolin-7-yl)oxy)propanoic acid (Example 90, 70 mg, 0.2 mmol) in CH₂Cl₂ (9 mL) was added DIPEA (0.2 mL, 1 mmol) followed by 1,4-thiazinane 1,1-dioxide (40 mg, 0.3 mmol). The reaction mixture was cooled to 0° C., and T₃P (0.2 mL, 0.3 mmol, 50% in EtOAc) was added. The reaction mixture was allowed to warm to ambient temperature and stirred for 16 h. The reaction mixture was quenched with water and extracted with CH₂Cl₂. The combined organic layers were washed with brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-(1,1-dioxidothiomorpholino)-1-oxopropan-2-yl)oxy)isoquinolin-1(2H)-one (Example 101, 48 mg). LCMS (ESI) Calcd. for C₂₂H₂₀ClFN₂O₅S: 478, found [M+H]⁺=479. ¹H NMR (400 MHz, DMSO-d₆) δ 11.50 (br s, 1H), 7.62-7.59 (m, 2H), 7.47 (br s, 1H), 7.34 (br s, 1H), 7.29-7.25 (m, 1H), 7.01 (br s, 1H), 6.96 (d, 1H), 5.58 (br s, 1H), 4.17 (br s, 2H), 3.89 (br s, 1H), 3.65 (br s, 1H), 3.42 (br s, 1H), 3.20-3.10 (m, 3H), 1.48 (d, 3H).

### Example 102: Synthesis of 4-(2-chloro-4-fluorophenyl)-7-(((R)-1-((R)-3-(methoxymethyl)morpholino)-1-oxopropan-2-yl)oxy)isoquinolin-1(2H)-one

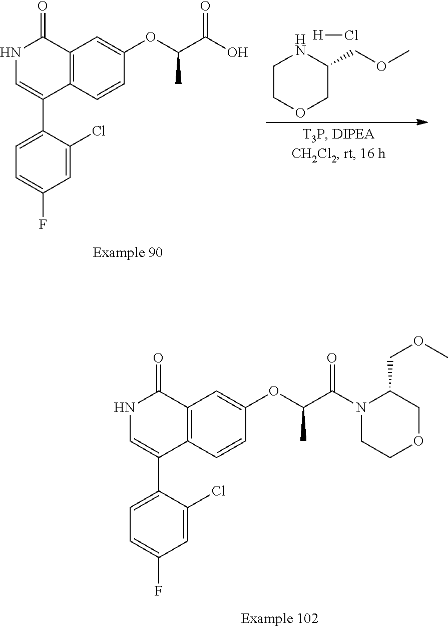

Synthesis of 4-(2-chloro-4-fluorophenyl)-7-(((R)-1-((R)-3-(methoxymethyl)morpholino)-1-oxopropan-2-yl)oxy)isoquinolin-1(2H)-one, Example 102: To a stirred solution of (R)-2-((4-(2-chloro-4-fluorophenyl)-1-oxo-1,2-dihydroisoquinolin-7-yl)oxy)propanoic acid (Example 90, 60 mg, 0.2 mmol) in CH₂Cl₂ (9 mL) was added DIPEA (0.1 mL, 0.8 mmol) followed by (R)-3-(methoxymethyl)morpholine hydrochloride (42 mg, 0.2 mmol). The reaction mixture was cooled to 0° C., and T₃P (0.1 mL, 0.2 mmol, 50% in EtOAc) was added. The reaction mixture was allowed to warm to ambient temperature and stirred for 16 h. The reaction mixture was quenched with ice cold water and extracted with CH₂Cl₂. The combined organic layers were washed with brine, dried over anhydrous Na₂SO₄, filtered, and concentrated under reduced pressure. The product was purified by reverse phase prep-HPLC and lyophilized to afford 4-(2-chloro-4-fluorophenyl)-7-(((R)-1-((R)-3-(methoxymethyl)morpholino)-1-oxopropan-2-yl)oxy)isoquinolin-1(2H)-one (Example 102, 35 mg). LCMS (ESI) Calcd. for C₂₄H₂₄ClFN₂O₅: 474, found [M+H]⁺=475. ¹H NMR (400 MHz, DMSO-d₆) δ 11.45-11.39 (m, 1H), 7.62-7.59 (m, 2H), 7.51-7.46 (m, 1H), 7.36-7.32 (m, 1H), 7.26-7.22 (m, 1H), 7.00-6.98 (m, 1H), 6.94 (d, 1H), 5.50-5.30 (m, 1H), 4.33 (br s, 1H), 4.09-3.96 (m, 1H), 3.85-3.78 (m, 3H), 3.72-3.59 (m, 2H), 3.47-3.34 (m, 2H), 3.23 (s, 3H), 1.48 (d, 3H).

### Example 103: Synthesis of 4-(2-chloro-4-fluorophenyl)-7-(((R)-1-((S)-3-(methoxymethyl)morpholino)-1-oxopropan-2-yl)oxy)isoquinolin-1(2H)-one

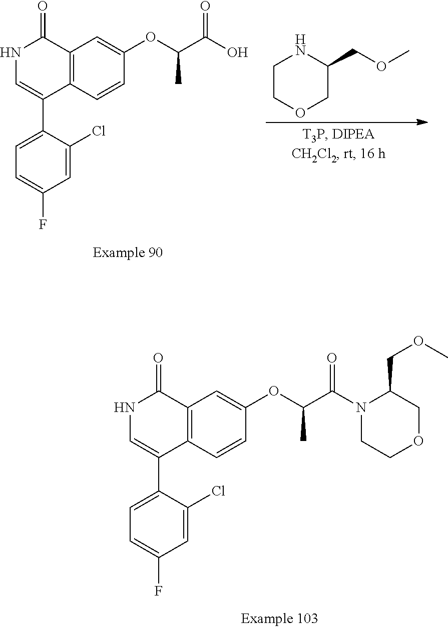

Synthesis of 4-(2-chloro-4-fluorophenyl)-7-(((R)-1-((S)-3-(methoxymethyl)morpholino)-1-oxopropan-2-yl)oxy)isoquinolin-1(2H)-one, Example 103: To a stirred solution of (R)-2-((4-(2-chloro-4-fluorophenyl)-1-oxo-1,2-dihydroisoquinolin-7-yl)oxy)propanoic acid (Example 90, 60 mg, 0.2 mmol) in CH₂Cl₂ (9 mL) was added DIPEA (0.1 mL, 0.8 mmol) followed by (S)-3-(methoxymethyl)morpholine (35 mg, 0.2 mmol). The reaction mixture was cooled to 0° C., and T₃P (0.14 mL, 0.2 mmol, 50% in EtOAc) was added. The reaction mixture was allowed to warm to ambient temperature and stirred for 16 h. The reaction mixture was quenched with water and extracted with CH₂Cl₂. The combined organic layers were washed with brine, dried over anhydrous Na₂SO₄, filtered, and concentrated under reduced pressure. The product was purified by reverse phase prep-HPLC and lyophilized to afford 4-(2-chloro-4-fluorophenyl)-7-(((R)-1-((S)-3-(methoxymethyl)morpholino)-1-oxopropan-2-yl)oxy)isoquinolin-1(2H)-one (Example 103, 48 mg). LCMS (ESI) Calcd. for C₂₄H₂₄ClFN₂O₅: 474, found [M+H]⁺=475. ¹H NMR (400 MHz, DMSO-d₆) δ 11.39 (br s, 1H), 7.66-7.59 (m, 2H), 7.50-7.45 (m, 1H), 7.36-7.24 (m, 2H), 7.00-6.90 (m, 2H), 5.50-5.33 (m, 1H), 4.37 (br s, 1H), 4.04 (br s, 2H), 3.85 (br s, 1H), 3.52 (br s, 1H), 3.41 (br s, 1H), 3.35-2.85 (m, 6H), 1.50-1.41 (m, 3H).

### Example 104: Synthesis of 4-(2-chloro-4-fluorophenyl)-7-(((R)-1-((R)-2-(hydroxymethyl)morpholino)-1-oxopropan-2-yl)oxy)isoquinolin-1(2H)-one

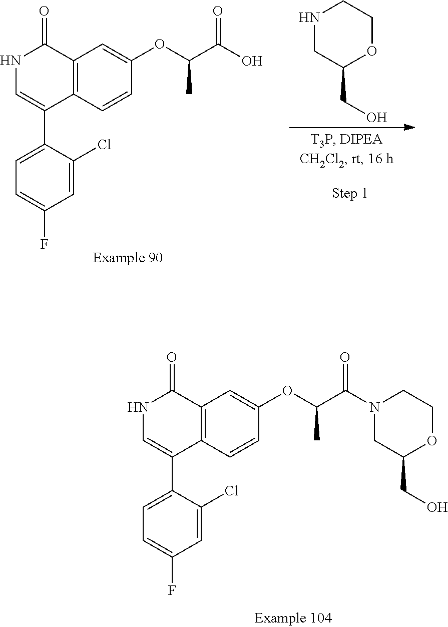 [SMILES: C[C@@H](Oc1ccc2c(-c3ccc(F)cc3Cl)c[nH]c(=O)c2c1)C(=O)O ; OC[C@H]1CNCCO1 ; C[C@@H](Oc1ccc2c(-c3ccc(F)cc3Cl)c[nH]c(=O)c2c1)C(=O)N1CCO[C@@H](CO)C1]

Synthesis of 4-(2-chloro-4-fluorophenyl)-7-(((R)-1-((R)-2-(hydroxymethyl)morpholino)-1-oxopropan-2-yl)oxy)isoquinolin-1(2H)-one, Example 104: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (9 mL) was added DIPEA (0.2 mL, 1.1 mmol) followed by (R)-morpholin-2-ylmethanol (39 mg, 0.3 mmol). The reaction mixture was cooled to 0° C., and T₃P (0.2 mL, 0.3 mmol, 50% in EtOAc) was added. The reaction mixture was allowed to warm to ambient temperature and stirred for 16 h. The reaction mixture was quenched with water and extracted with CH₂Cl₂. The combined organic layers were washed with brine, dried over anhydrous. Na₂SO₄, filtered, and concentrated under reduced pressure. The product was purified by reverse phase prep-HPLC and lyophilized to afford 4-(2-chloro-4-fluorophenyl)-7-(((R)-1-((R)-2-(hydroxymethyl)morpholino)-1-oxopropan-2-yl)oxy)isoquinolin-1(2H)-one (Example 104, 45 mg). LCMS (ESI) Calcd. for C₂₃H₂₂ClFN₂O₅: 460, found [M+H]⁺=461. ¹H NMR (400 MHz, DMSO-d₆) δ 11.36 (br s, 1H), 7.58 (br s, 2H), 7.45 (br s, 1H), 7.33-7.31 (m, 1H), 7.23 (d, 1H), 6.97-6.91 (m, 2H), 5.41-5.37 (m, 1H), 4.76-4.72 (m, 1H), 4.04-4.24 (m, 2H), 3.85-3.83 (m, 2H), 3.66 (br s, 1H), 3.42-3.39 (m, 2H), 3.01-2.70 (m, 2H), 1.44-1.42 (m, 3H).

### Example 105: Synthesis of 7-(((2R)-1-(8-oxa-3-azabicyclo[3.2.1]octan-3-yl)-1-oxopropan-2-yl)oxy)-4-(2-chloro-4-fluorophenyl)isoquinolin-1(2H)-one

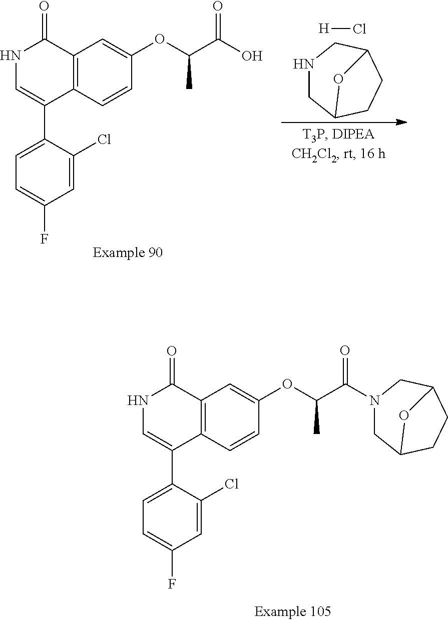

Synthesis of 7-(((2R)-1-(8-oxa-3-azabicyclo[3.2.1]octan-3-yl)-1-oxopropan-2-yl)oxy)-4-(2-chloro-4-fluorophenyl)isoquinolin-1(2H)-one, Example 105: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (9 mL) was added DIPEA (0.2 mL, 1.1 mmol) followed by 8-oxa-3-azabicyclo[3.2.1]octane hydrochloride (50 mg, 0.3 mmol). The reaction mixture was cooled to 0° C., and T₃P (0.19 mL, 0.3 mmol, 50% in EtOAc) was added. The reaction mixture was allowed to warm to ambient temperature and stirred for 16 h. The reaction mixture was quenched with water and extracted with CH₂Cl₂. The combined organic layers were washed with brine, dried over anhydrous Na₂SO₄, filtered, and concentrated under reduced pressure. The product was purified by reverse phase prep-HPLC and lyophilized to afford 7-(((2R)-1-(8-oxa-3-azabicyclo[3.2.1]octan-3-yl)-1-oxopropan-2-yl)oxy)-4-(2-chloro-4-fluorophenyl)isoquinolin-1(2H)-one (Example 105, 55 mg). LCMS (ESI) Calcd. for C₂₄H₂₂ClFN₂O₄: 456, found [M+H]⁺=457. ¹H NMR (400 MHz, DMSO-d₆) δ 11.41 (br s, 1H), 7.62-7.57 (m, 2H), 7.48 (t, 1H), 7.36-7.32 (m, 1H), 7.27-7.24 (m, 1H), 7.00-6.92 (m, 2H), 5.48-5.30 (m, 1H), 4.32 (br s, 2H), 3.89-3.80 (m, 2H), 3.42 (br s, 1H), 2.83-2.80 (m, 1H), 2.22 (br s, 1H), 1.86-1.72 (m, 3H), 1.49-1.39 (m, 3H).

### Example 106: Synthesis of 4-(2-chloro-4-fluorophenyl)-7-(((R)-1-((S)-2-(hydroxymethyl)morpholino)-1-oxopropan-2-yl)oxy)isoquinolin-1(2H)-one

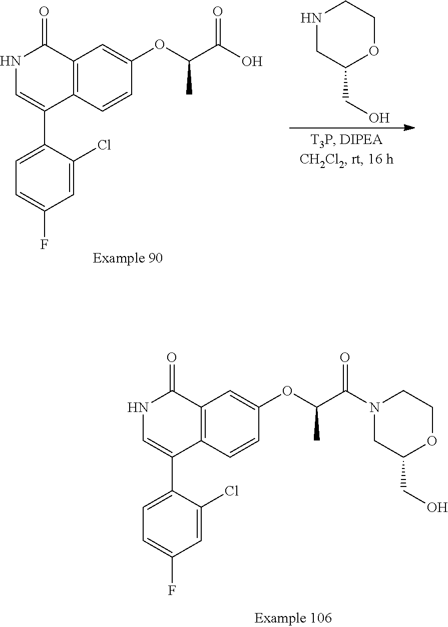 [SMILES: C[C@@H](Oc1ccc2c(-c3ccc(F)cc3Cl)c[nH]c(=O)c2c1)C(=O)O ; OC[C@@H]1CNCCO1 ; C[C@@H](Oc1ccc2c(-c3ccc(F)cc3Cl)c[nH]c(=O)c2c1)C(=O)N1CCO[C@H](CO)C1]

Synthesis of 4-(2-chloro-4-fluorophenyl)-7-(((R)-1-((S)-2-(hydroxymethyl)morpholino)-1-oxopropan-2-yl)oxy)isoquinolin-1(2H)-one, Example 106: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (9 mL) was added DIPEA (0.2 mL, 1.1 mmol) followed by (S)-morpholin-2-ylmethanol (40 mg, 0.3 mmol). The reaction mixture was cooled to 0° C., and T₃P (0.2 mL, 0.3 mmol, 50% in EtOAc) was added. The reaction mixture was allowed to warm to ambient temperature and stirred for 16 h. The reaction mixture was quenched with water and extracted with CH₂Cl₂. The combined organic layers were washed with brine, dried over anhydrous Na₂SO₄, filtered, and concentrated under reduced pressure. The product was purified by reverse phase prep-HPLC and lyophilized to afford 4-(2-chloro-4-fluorophenyl)-7-(((R)-1-((S)-2-(hydroxymethyl)morpholino)-1-oxopropan-2-yl)oxy)isoquinolin-1(2H)-one (Example 106, 32 mg). LCMS (ESI) Calcd. for C₂₃H₂₂ClFN₂O₅: 460, found [M+H]⁺=461. ¹H NMR (400 MHz, DMSO-d₆) δ 11.42 (br s, 1H), 7.59 (br s, 2H), 7.48-7.46 (m, 1H), 7.36-7.31 (m, 1H), 7.26-7.24 (m, 1H), 7.00-6.91 (m, 2H), 5.52-5.50 (m, 1H), 4.79-4.77 (m, 1H), 4.33-4.29 (m, 1H), 4.11-4.06 (m, 1H), 3.38-3.79 (m, 2H), 3.43-3.42 (m, 2H), 3.22-2.72 (m, 3H), 1.49-1.43 (m, 3H).

### Example 107: Synthesis of (R)-4-(2-chloro-4-fluorophenyl)-7-((1-(3,3-dimethylmorpholino)-1-oxopropan-2-yl)oxy)isoquinolin-1(2H)-one

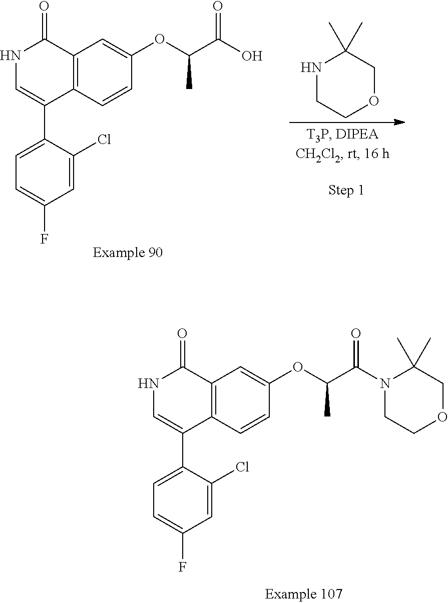

Synthesis of (R)-4-(2-chloro-4-fluorophenyl)-7-((1-(3,3-dimethylmorpholino)-1-oxopropan-2-yl)oxy)isoquinolin-1(2H)-one, Example 107: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (9 mL) was added DIPEA (0.2 mL, 1.1 mmol) followed by 3,3-dimethylmorpholine (40 mg, 0.3 mmol). The reaction mixture was cooled to 0° C., and T₃P (0.2 mL, 0.3 mmol, 50% in EtOAc) was added. The reaction mixture was allowed to warm up to ambient temperature and stirred for 16 h. The reaction mixture was quenched with water and extracted with CH₂Cl₂. The combined organic layers were washed with brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-(3,3-dimethylmorpholino)-1-oxopropan-2-yl)oxy)isoquinolin-1(2H)-one (Example 107, 47 mg). LCMS (ESI) Calcd. for C₂₄H₂₄ClFN₂O₄: 458, found [M−H]⁻=457. ¹H NMR (400 MHz, DMSO-d₆) δ 11.38 (br s, 1H), 7.62-7.58 (m, 2H), 7.48-7.46 (m, 1H), 7.36-7.33 (m, 1H), 7.24-7.21 (m, 1H), 6.98 (s, 1H), 6.94-6.92 (m, 1H), 5.30-5.25 (m, 1H), 3.83-3.79 (m, 1H), 3.69-3.65 (m, 2H), 3.62-3.56 (m, 1H), 3.39-3.36 (m, 1H), 1.43 (d, 3H), 1.28-1.25 (m, 6H).

### Examples 108-110: Synthesis of 2-((4-(4-fluoro-2,6-dimethylphenyl)-1-oxo-1,2-dihydroisoquinolin-7-yl)oxy)acetonitrile, 2-((4-(4-fluoro-2,6-dimethylphenyl)-1-oxo-1,2-dihydroisoquinolin-7-yl)oxy)acetamide, and 2-((4-(4-fluoro-2,6-dimethylphenyl)-1-oxo-1,2-dihydroisoquinolin-7-yl)oxy)acetic acid

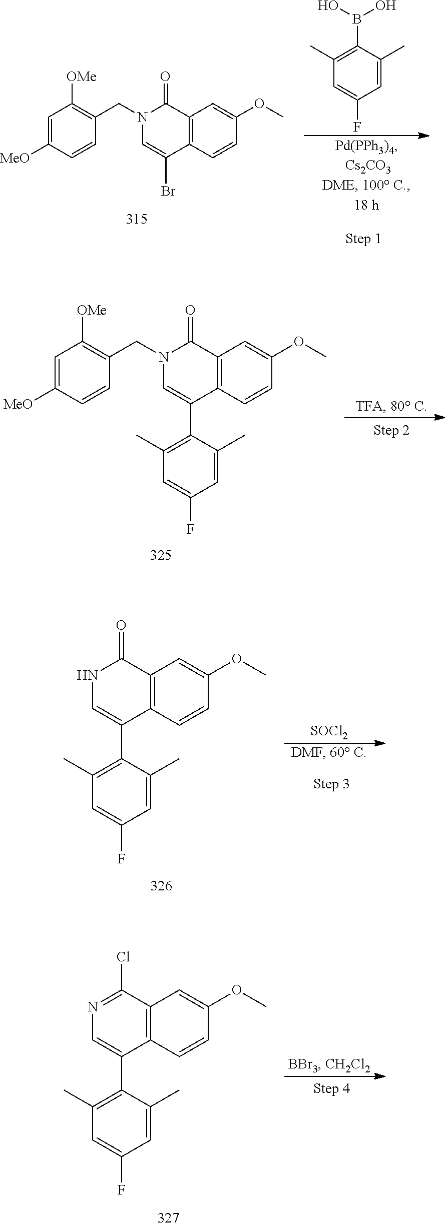

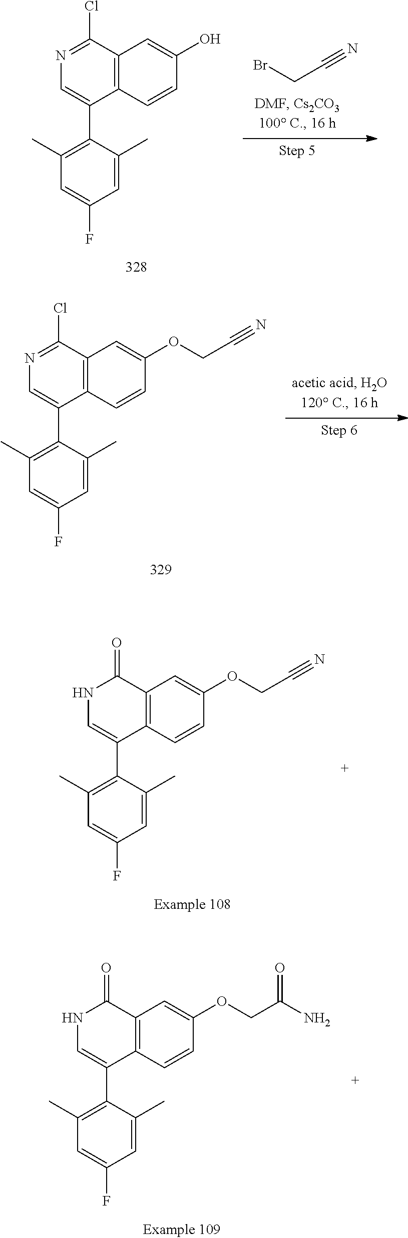

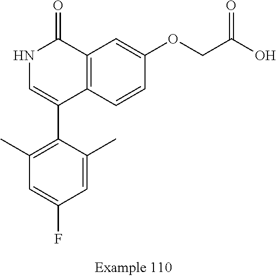 [SMILES: Cc1cc(F)cc(C)c1-c1c[nH]c(=O)c2cc(OCC(=O)O)ccc12]

Synthesis of 2-(2,4-dimethoxybenzyl)-4-(4-fluoro-2,6-dimethylphenyl)-7-methoxyisoquinolin-1(2H)-one, 325 [Step 1]: To a degassed solution of 4-bromo-2-(2,4-dimethoxybenzyl)-7-methoxyisoquinolin-1(2H)-one (315, 600 mg, 1.5 mmol) was added a solution of (4-fluoro-2,6-dimethyl-phenyl)boronic acid (400 mg, 2.4 mmol) in 1,2-dimethoxyethane (15 mL). Cs₂CO₃ (960 mg, 2.9 mmol) and tetrakis(triphenylphosphine)palladium(0) (170 mg, 0.15 mmol) were added to the reaction mixture under N₂ atmosphere. The mixture was stirred at 100° C. for 18 h. The reaction was allowed to warm to ambient temperature, quenched with ice cold water, and extracted with EtOAc (×2). The combined organic extracts were washed with ice cold brine (50 mL), dried over anhydrous Na₂SO₄, filtered, and concentrated under reduced pressure. The product was purified by column chromatography over silica gel to afford 2-(2,4-dimethoxybenzyl)-4-(4-fluoro-2,6-dimethylphenyl)-7-methoxyisoquinolin-1(2H)-one (325, 370 mg). LCMS (ESI) Calcd. for C₂₇H₂₆FNO₄: 447, found [M+H]⁺=448.

Synthesis of 4-(4-fluoro-2,6-dimethylphenyl)-7-methoxyisoquinolin-1(2H)-one, 326 [Step 2]: To a sealed tube was added 2-(2,4-dimethoxybenzyl)-4-(4-fluoro-2,6-dimethylphenyl)-7-methoxyisoquinolin-1(2H)-one (325, 370 mg, 0.8 mmol) and trifluoroacetic acid (6.3 mL, 82.7 mmol). The reaction mixture was heated at 80° C. for 16 h. The reaction mixture was concentrated under reduced pressure. The reaction mixture was diluted in EtOAc, washed with brine, dried over anhydrous Na₂SO₄, and concentrated under reduced pressure. The product was purified by column chromatography to afford 4-(4-fluoro-2,6-dimethylphenyl)-7-methoxyisoquinolin-1(2H)-one (326, 150 mg). LCMS (ESI) Calcd. for C₁₈H₁₆FNO₂: 297, found [M+H]⁺=298.

Synthesis of 1-chloro-4-(4-fluoro-2,6-dimethylphenyl)-7-methoxyisoquinoline, 327 [Step 3]: To a stirred solution of 4-(4-fluoro-2,6-dimethylphenyl)-7-methoxyisoquinolin-1(2R)-one (326, 150 mg, 0.5 mmol) in SOCl₂ (3.7 mL, 50.5 mmol) was added DMF (0.04 mL, 0.505 mmol) at ambient temperature. The reaction mixture was heated at 60° C. for 18 h. The reaction mixture was concentrated under reduced pressure. The reaction mixture was cooled to 0° C., quenched with ice cold water, and extracted with EtOAc. The combined organic layers were washed with brine, dried over anhydrous Na₂SO₄, filtered, and concentrated under reduced pressure. The product was purified by column chromatography to afford 1-chloro-4-(4-fluoro-2,6-dimethylphenyl)-7-methoxyisoquinoline (327, 120 mg). LCMS (ESI) Calcd. for C₁₈H₁₅ClFNO: 315, found [M+H]⁺=316.

Synthesis of 1-chloro-4-(4-fluoro-2,6-dimethylphenyl)isoquinolin-7-ol, 328 [Step 4]: To a stirred solution 1-chloro-4-(4-fluoro-2,6-dimethylphenyl)-7-methoxyisoquinoline (327, 120 mg, 0.4 mmol) in CH₂Cl₂ (4 mL) was added dropwise BBr₃ (285 mg, 1.14 mmol, 1M in CH₂Cl₂) at 0° C. The reaction mixture was warmed to ambient temperature and stirred for 16 h. The reaction mixture was concentrated under reduced pressure, cooled to 0° C., and quenched with MeOH. The reaction mixture was concentrated under reduced pressure. The reaction mixture was extracted in EtOAc, washed with brine, dried over anhydrous Na₂SO₄, filtered, and concentrated under reduced pressure to afford 1-chloro-4-(4-fluoro-2,6-dimethylphenyl)isoquinolin-7-ol (328, 100 mg). The product was used in the next step without further purification. LCMS (ESI) Calcd. for C₁₇H₁₃ClFNO: 301, found [M+H]⁺=302.

Synthesis of 2-((1-chloro-4-(4-fluoro-2,6-dimethylphenyl)isoquinolin-7-yl)oxy)acetonitrile, 329 [Step 5]: To a stirred solution of 1-chloro-4-(4-fluoro-2,6-dimethylphenyl)isoquinolin-7-ol (328, 100 mg, 0.3 mmol) and 2-bromoacetonitrile (60 mg, 0.5 mmol) in DMF (4 mL) was added Cs₂CO₃ (325 mg, 1.0 mmol) at ambient temperature. The reaction mixture was stirred at 100° C. for 16 h. The reaction mixture was quenched with water and extracted with EtOAc. The combined organic extracts were dried over anhydrous Na₂SO₄, filtered, and concentrated under reduced pressure. The product was purified by flash chromatography to afford 2-((1-chloro-4-(4-fluoro-2,6-dimethylphenyl)isoquinolin-7-yl)oxy)acetonitrile (329, 100 mg). LCMS (ESI) Calcd. for C₁₉H₁₄ClFN₂O: 340, found [M+H]⁺=341.

Synthesis of 2-((4-(4-fluoro-2,6-dimethylphenyl)-1-oxo-1,2-dihydroisoquinolin-7-yl)oxy)acetonitrile, Example 108, 2-((4-(4-fluoro-2,6-dimethylphenyl)-1-oxo-1,2-dihydroisoquinolin-7-yl)oxy)acetamide, Example 109, and 2-((4-(4-fluoro-2,6-dimethylphenyl)-1-oxo-1,2-dihydroisoquinolin-7-yl)oxy)acetic acid, Example 110 [Step 6]: To a stirred solution of 2-((1-chloro-4-(4-fluoro-2,6-dimethylphenyl)isoquinolin-7-yl)oxy)acetonitrile (329, 100 mg, 0.3 mmol) in acetic acid (2.5 mL, 44.0 mmol) was added water (0.5 mL, 29.3 mmol). The reaction mixture was heated at 120° C. for 16 h. The reaction mixture was quenched with water and extracted with EtOAc. The combined organic extracts were dried over anhydrous Na₂SO₄, filtered, and concentrated under reduced pressure. The products were purified by reverse phase prep-HPLC to afford 2-((4-(4-fluoro-2,6-dimethylphenyl)-1-oxo-1,2-dihydroisoquinolin-7-yl)oxy)acetonitrile (Example 108, 6.0 mg), 2-((4-(4-fluoro-2,6-dimethylphenyl)-1-oxo-1,2-dihydroisoquinolin-7-yl)oxy)acetamide (Example 109, 7.0 mg), and 2-((4-(4-fluoro-2,6-dimethylphenyl)-1-oxo-1,2-dihydroisoquinolin-7-yl)oxy)acetic acid (Example 110, 7.0 mg).

Example 108: 2-((4-(4-fluoro-2,6-dimethylphenyl)-1-oxo-1,2-dihydroisoquinolin-7-yl)oxy)acetonitrile: LCMS (ESI) Calcd. for C₁₉H₁₅FN₂O₂: 322, found [M+H]⁺=323. ¹H NMR (400 MHz, DMSO-d₆) δ 11.51 (br s, 1H), 7.86 (d, 1H), 7.36-7.33 (m, 1H), 7.05 (d, 2H), 6.93-6.91 (m, 1H), 6.83 (d, 1H), 5.32 (s, 2H), 1.97 (s, 6H).

Example 109: 2-((4-(4-fluoro-2,6-dimethylphenyl)-1-oxo-1,2-dihydroisoquinolin-7-yl)oxy)acetamide: LCMS (ESI) Calcd. for C₁₉H₁₇FN₂O₃: 340, found [M−H]⁻=339. ¹H NMR (400 MHz, DMSO-d₆) δ 11.39 (br s, 1H), 7.67 (d, 1H), 7.63 (br s, 1H), 7.40 (br s, 1H), 7.32-7.29 (m, 1H), 7.04 (d, 2H), 6.85 (s, 1H), 6.79 (d, 1H), 4.55 (s, 2H), 1.97 (s, 6H).

Example 110: 2-((4-(4-fluoro-2,6-dimethylphenyl)-1-oxo-1,2-dihydroisoquinolin-7-yl)oxy)acetic acid: LCMS (ESI) Calcd. for C₁₉H₁₆FNO₄: 341, found [M+H]⁺=342. ¹H NMR (400 MHz, DMSO-d₆) δ 11.34 (br s, 1H), 7.56 (d, 1H), 7.21-7.18 (m, 1H), 7.03 (d, 2H), 6.81 (m, 1H), 6.73 (d, 1H), 4.46 (s, 2H), 1.97 (6H).

### Example 111: Synthesis of (R)-2-((4-(2,6-dimethylphenyl)-1-oxo-1,2-dihydroisoquinolin-7-yl)oxy)propanoic acid

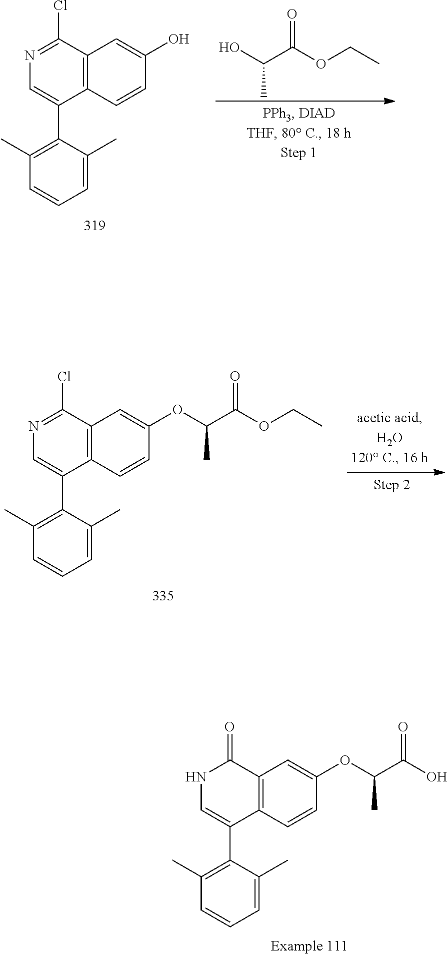

Synthesis of ethyl (R)-2-((1-chloro-4-(2,6-dimethylphenyl)isoquinolin-7-yl)oxy)propanoate, 335 [Step 1]: To a stirred solution of 1-chloro-4-(2,6-dimethylphenyl)isoquinolin-7-ol (319, 90 mg, 0.3 mmol) and ethyl (S)-2-hydroxypropanoate (56 mg, 0.5 mmol) in THF (10 mL) was added PPh₃ (250 mg, 0.9 mmol). The reaction mixture was cooled to 0° C., and DIAD (0.2 mL, 0.9 mmol) was added dropwise. The reaction mixture was stirred for 5 min. at 0° C. and then 80° C. for 18 h. The reaction mixture was diluted with EtOAc, washed with water, brine, dried over anhydrous Na₂SO₄, and concentrated under reduced pressure. The product was purified by flash chromatography to afford ethyl (R)-2-((1-chloro-4-(2,6-dimethylphenyl)isoquinolin-7-yl)oxy)propanoate (335, 100 mg). LCMS (ESI) Calcd. for C₂₂H₂₂ClNO₃: 383, found [M+H]⁺=384.

Synthesis of (R)-2-((4-(2,6-dimethylphenyl)-1-oxo-1,2-dihydroisoquinolin-7-yl)oxy)propanoic acid, Example 111 [Step 2]: To a stirred solution of ethyl (R)-2-((1-chloro-4-(2,6-dimethylphenyl)isoquinolin-7-yl)oxy)propanoate (335, 100 mg, 0.3 mmol) in acetic acid (2.2 mL, 39.1 mmol) was added water (0.5 mL, 26.1 mmol). The reaction mixture was heated at 120° C. for 16 h. The reaction mixture was concentrated under reduced pressure. The reaction mixture was diluted with EtOAc, washed with brine, dried over anhydrous Na₂SO₄, filtered, and concentrated under reduced pressure. The product was purified by prep-HPLC to afford (R)-2-((4-(2,6-dimethylphenyl)-1-oxo-1,2-dihydroisoquinolin-7-yl)oxy)propanoic acid (Example 111, 40 mg). LCMS (ESI) Calcd. for C₂₀H₁₉NO₄: 337, found [M+H]⁺=338. ¹H NMR (400 MHz, DMSO-d₆) δ 13.15 (br s, 1H), 11.36 (br s, 1H), 7.58 (d, 1H), 7.26-7.22 (m, 2H), 7.18-7.16 (m, 2H), 6.84-6.83 (m, 1H), 6.76 (d, 1H), 4.92 (q, 1H), 1.97-1.95 (m, 6H), 1.54 (d, 3H).

### Example 112: Synthesis of (S)-4-((R)-2-((4-(2-chloro-4-fluorophenyl)-1-oxo-1,2-dihydroisoquinolin-7-yl)oxy)propanoyl)morpholine-3-carboxylic acid

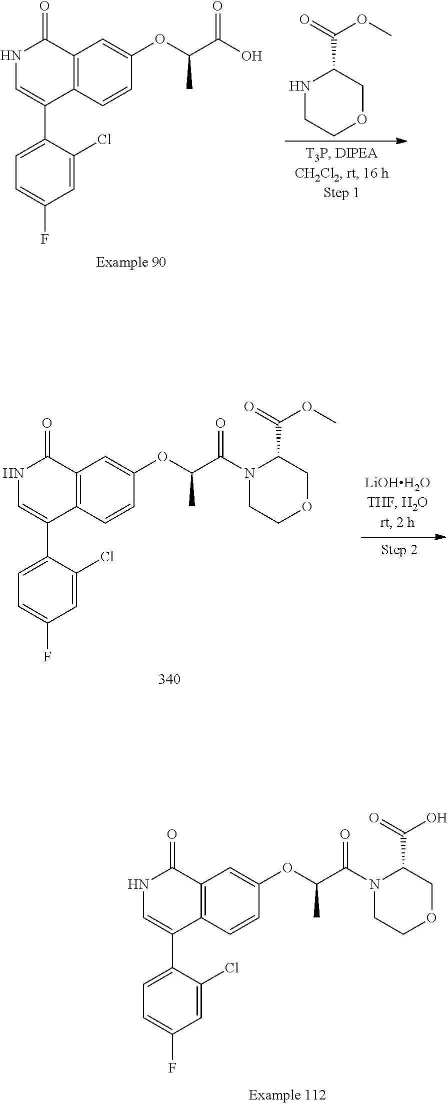

Synthesis of methyl (S)-4-((R)-2-((4-(2-chloro-4-fluorophenyl)-1-oxo-1,2-dihydroisoquinolin-7-yl)oxy)propanoyl)morpholine-3-carboxylate, 340 [Step 1]: To a stirred solution of (R)-2-((4-(2-chloro-4-fluorophenyl)-1-oxo-1,2-dihydroisoquinolin-7-yl)oxy)propanoic acid (Example 90, 100 mg, 0.3 mmol) in CH₂Cl₂ (9 mL) was added DIPEA (0.25 mL, 1.4 mmol) and methyl (S)-morpholine-3-carboxylate (60 mg, 0.4 mmol). The reaction mixture was cooled to 0° C., and T₃P (0.24 mL, 0.4 mmol, 50% in EtOAc) was added. The reaction mixture was allowed to warm to ambient temperature and stirred for 16 h. The reaction mixture was quenched with water and extracted with CH₂Cl₂ (×3). The combined organic extracts were washed with brine, dried over anhydrous Na₂SO₄, filtered, and concentrated under reduced pressure. The product was purified by flash chromatography to afford (S)-4-((R)-2-((4-(2-chloro-4-fluorophenyl)-1-oxo-1,2-dihydroisoquinolin-7-yl)oxy)propanoyl)morpholine-3-carboxylate (340, 130 mg). LCMS (ESI) Calcd. for C₂₄H₂₂ClFN₂O₆: 488, found [M+H]⁺=489.

Synthesis of (S)-4-((R)-2-((4-(2-chloro-4-fluorophenyl)-1-oxo-1,2-dihydroisoquinolin-7-yl)oxy)propanoyl)morpholine-3-carboxylic acid, Example 112 [Step 2]: To a stirred solution of (S)-4-((R)-2-((4-(2-chloro-4-fluorophenyl)-1-oxo-1,2-dihydroisoquinolin-7-yl)oxy)propanoyl)morpholine-3-carboxylate (340, 200 mg, 0.4 mmol) in THF (8 mL) was added an aq. solution (2 mL) of LiOH·H₂O (60 mg, 1.4 mmol). The reaction mixture was stirred for 2 h. at ambient temperature. The reaction mixture was concentrated under reduced pressure. The reaction mixture was diluted with water, acidified with 1M citric acid to pH=5, and extracted with EtOAc (×3). The combined organic extracts were washed with brine, dried over anhydrous Na₂SO₄, filtered, and concentrated under reduced pressure. The product was purified by reverse phase prep-HPLC and lyophilized to afford (S)-4-((R)-2-((4-(2-chloro-4-fluorophenyl)-1-oxo-1,2-dihydroisoquinolin-7-yl)oxy)propanoyl)morpholine-3-carboxylic acid (Example 112, 40 mg). LCMS (ESI) Calcd. for C₂₃H₂₀ClFN₂O₆: 474, found [M+H]⁺=475. ¹H NMR (400 MHz, DMSO-d₆) (at 100° C.) δ 11.11 (br s, 1H), 7.74 (br s, 1H), 7.53-7.44 (m, 2H), 7.32-7.24 (m, 2H), 6.97-6.94 (m, 2H), 5.35 (br s, 1H), 4.89-4.78 (m, 1H), 4.25 (br s, 1H), 3.98-3.82 (m, 3H), 3.54 (br s, 1H), 3.39 (br s, 2H), 1.51 (br s, 3H).

### Example 113: Synthesis of 4-(2-chloro-4-fluorophenyl)-7-(((R)-1-((R)-3-(hydroxymethyl)morpholino)-1-oxopropan-2-yl)oxy)isoquinolin-1(2H)-one

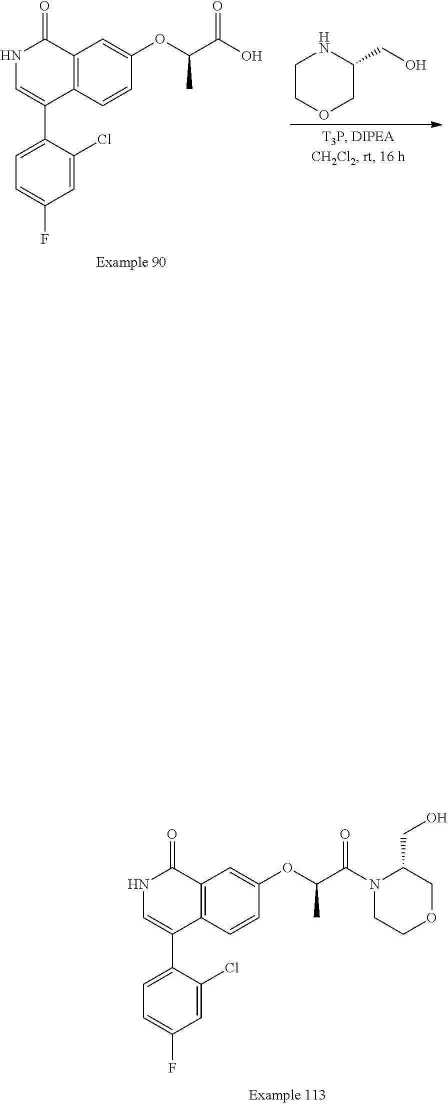 [SMILES: C[C@@H](Oc1ccc2c(-c3ccc(F)cc3Cl)c[nH]c(=O)c2c1)C(=O)O ; OC[C@@H]1COCCN1 ; C[C@@H](Oc1ccc2c(-c3ccc(F)cc3Cl)c[nH]c(=O)c2c1)C(=O)N1CCOC[C@H]1CO]

Synthesis of 4-(2-chloro-4-fluorophenyl)-7-(((R)-1-((R)-3-(hydroxymethyl)morpholino)-1-oxopropan-2-yl)oxy)isoquinolin-1(2H)-one, Example 113: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (5 mL) was added DIPEA (0.2 mL, 1.1 mmol) and (R)-morpholin-3-ylmethanol (2, 40 mg, 0.3 mmol). The reaction mixture was cooled to 0° C., and T₃P (0.2 mL, 0.3 mmol, 50% in EtOAc) was added. The reaction mixture was allowed to warm to ambient temperature and stirred for 16 h. The reaction mixture was quenched with water and extracted with CH₂Cl₂ (×3). The combined organic extracts were washed with brine, dried over anhydrous Na₂SO₄, filtered, and concentrated under reduced pressure. The product was purified by reverse phase prep-HPLC and lyophilized to afford 4-(2-chloro-4-fluorophenyl)-7-(((R)-1-((R)-3-(hydroxymethyl)morpholino)-1-oxopropan-2-yl)oxy)isoquinolin-1(2H)-one (Example 113, 47 mg). LCMS (ESI) Calcd. for C₂₃H₂₂ClFN₂O₅: 460, found [M−H]⁻=459. ¹H NMR (400 MHz, DMSO-d₆) (at 100° C.) δ 11.08 (br s, 1H), 7.69 (s, 1H), 7.52-7.44 (m, 2H), 7.32-7.24 (m, 2H), 6.99-6.94 (m, 2H), 5.34 (br s, 1H), 4.64 (br s, 1H), 4.08 (m, 2H), 3.96-3.81 (m, 3H), 3.70 (br s, 1H), 3.57 (br s, 1H), 3.37 (t, 2H), 1.52 (d, 3H).

### Example 114: Synthesis of 4-(2-chloro-4-fluorophenyl)-7-(((R)-1-((S)-3-(hydroxymethyl)morpholino)-1-oxopropan-2-yl)oxy)isoquinolin-1(2H)-one

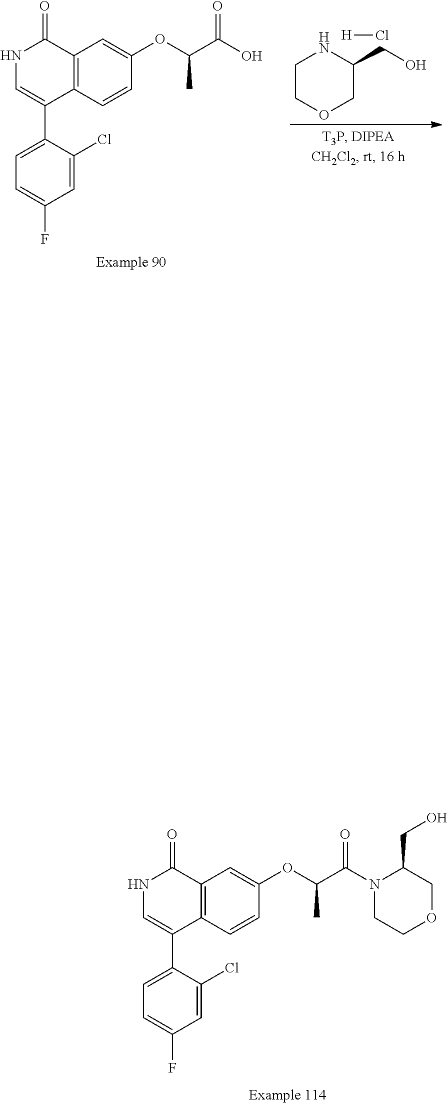

Synthesis of 4-(2-chloro-4-fluorophenyl)-7-(((R)-1-((S)-3-(hydroxymethyl)morpholino)-1-oxopropan-2-yl)oxy)isoquinolin-1(2H)-one, Example 114: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (9 mL) was added DIPEA (0.2 mL, 1.1 mmol) and (S)-morpholin-3-ylmethanol hydrochloride (2, 50 mg, 0.3 mmol). The reaction mixture was cooled to 0° C., and T₃P (0.2 mL, 0.3 mmol, 50% in EtOAc) was added. The reaction mixture was allowed to warm to ambient temperature and stirred for 16 h. The reaction mixture was quenched with water and extracted with CH₂Cl₂ (×3). The combined organic extracts were washed with brine, dried over anhydrous Na₂SO₄, filtered, and concentrated under reduced pressure. The product was purified by reverse phase prep-HPLC and lyophilized to afford 4-(2-chloro-4-fluorophenyl)-7-(((R)-1-((S)-3-(hydroxymethyl)morpholino)-1-oxopropan-2-yl)oxy)isoquinolin-1(2H)-one (Example 114, 60 mg). LCMS (ESI) Calcd. for C₂₃H₂₂ClFN₂O₅: 460, found [M−H]⁻=459. ¹H NMR (400 MHz, DMSO-d₆) δ 11.38 (br s, 1H), 7.70-7.59 (m, 2H), 7.47 (t, 1H), 7.38-7.24 (m, 2H), 6.99-6.87 (m, 2H), 5.46-4.83 (m, 2H), 4.71-2.97 (m, 9H), 1.51-1.43 (m, 3H).

### Example 115: Synthesis of (R)-4-((R)-2-((4-(2-chloro-4-fluorophenyl)-1-oxo-1,2-dihydroisoquinolin-7-yl)oxy)propanoyl)morpholine-3-carboxylic acid

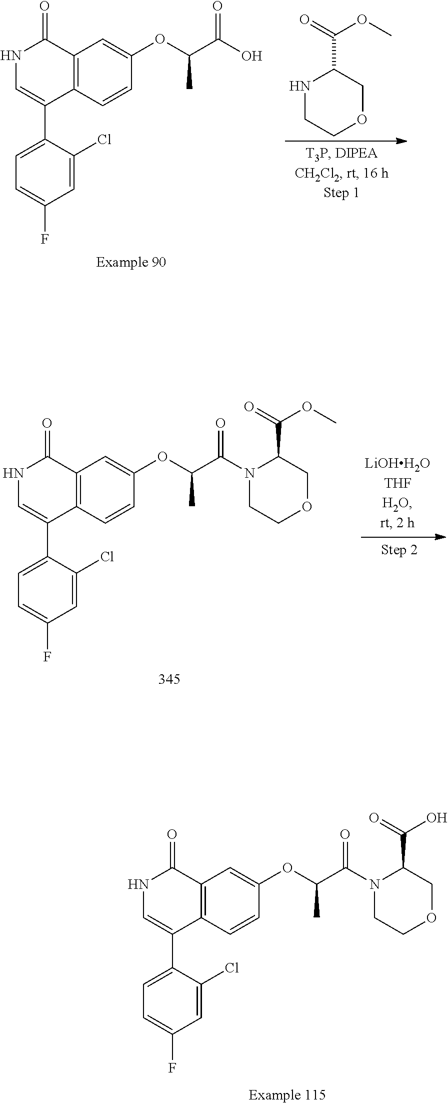

Synthesis of methyl (R)-4-((R)-2-((4-(2-chloro-4-fluorophenyl)-1-oxo-1,2-dihydroisoquinolin-7-yl)oxy)propanoyl)morpholine-3-carboxylate, 345 [Step 1]: To a stirred solution of (R)-2-((4-(2-chloro-4-fluorophenyl)-1-oxo-1,2-dihydroisoquinolin-7-yl)oxy)propanoic acid (Example 90, 100 mg, 0.3 mmol) in CH₂Cl₂ (9 mL) was added DIPEA (0.25 mL, 1.4 mmol) and methyl (R)-morpholine-3-carboxylate (2, 60 mg, 0.4 mmol). The reaction mixture was cooled to 0° C., and T₃P (0.25 mL, 0.4 mmol, 50% in EtOAc) was added. The reaction mixture was allowed to warm to ambient temperature and stirred for 16 h. The reaction mixture was quenched with water and extracted with CH₂Cl₂ (×3). The combined organic extracts were washed with brine, dried over anhydrous Na₂SO₄, filtered, and concentrated under reduced pressure. The product was purified by flash chromatography to afford methyl (R)-4-((R)-2-((4-(2-chloro-4-fluorophenyl)-1-oxo-1,2-dihydroisoquinolin-7-yl)oxy)propanoyl)morpholine-3-carboxylate (345, 110 mg). LCMS (ESI) Calcd. for C₂₄H₂₂ClFN₂O₆: 488, found [M+H]+=489.

Synthesis of (R)-4-((R)-2-((4-(2-chloro-4-fluorophenyl)-1-oxo-1,2-dihydroisoquinolin-7-yl)oxy)propanoyl)morpholine-3-carboxylic acid, Example 115 [Step 2]: To a stirred solution of (R)-4-((R)-2-((4-(2-chloro-4-fluorophenyl)-1-oxo-1,2-dihydroisoquinolin-7-yl)oxy)propanoyl)morpholine-3-carboxylate (345, 130 mg, 0.3 mmol) in THF (8 mL) was added an aq. solution (2 mL) of LiOH·H₂O (40 mg, 0.9 mmol). The reaction mixture was stirred at ambient temperature for 2 h. The reaction mixture was concentrated under reduced pressure. The reaction mixture was diluted with water, acidified with 1M citric acid to pH=5, and extracted with EtOAc (×3). The combined organic extracts were washed with brine, dried over anhydrous Na₂SO₄, filtered, and concentrated under reduced pressure. The product was purified by reverse phase prep-HPLC and lyophilized to afford (R)-4-((R)-2-((4-(2-chloro-4-fluorophenyl)-1-oxo-1,2-dihydroisoquinolin-7-yl)oxy)propanoyl)morpholine-3-carboxylic acid (Example 115, 52 mg). LCMS (ESI) Calcd. for C₂₃H₂₀ClFN₂O₆: 474, found [M+H]⁺=475. ¹H NMR (400 MHz, DMSO-d₆) δ 13.12 (br s, 1H), 11.40-11.39 (m, 1H), 7.72-7.60 (m, 2H), 7.50-7.45 (t, 1H), 7.34 (t, 1H), 7.26 (d, 1H), 6.99-6.98 (m, 1H), 6.94-6.92 (m, 1H), 5.60-5.57 (m, 1H), 4.78-2.92 (m, 7H), 1.45-1.44 (m, 3H).

### Example 116: Synthesis of (R)-7-((1-(4-acetylpiperazin-1-yl)-1-oxopropan-2-yl)oxy)-4-(2-chloro-4-fluorophenyl)isoquinolin-1(2H)-one

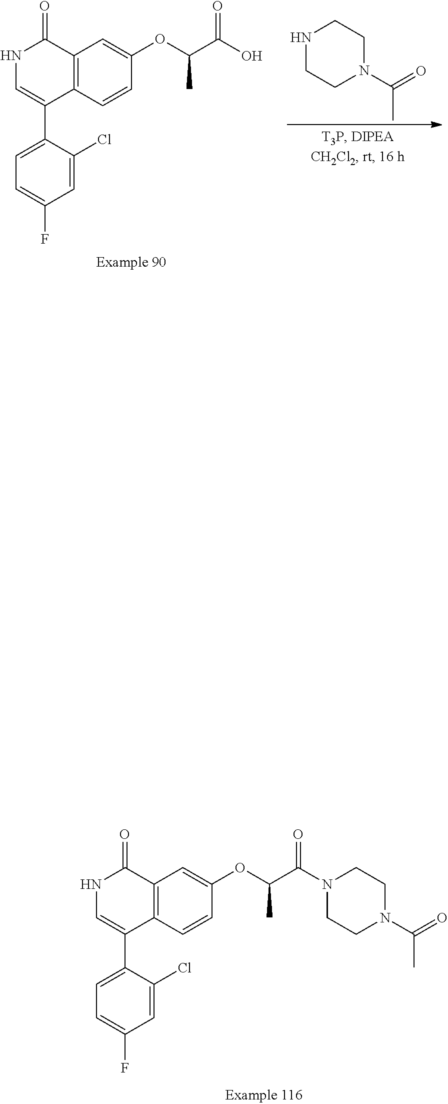

Synthesis of (R)-7-((1-(4-acetylpiperazin-1-yl)-1-oxopropan-2-yl)oxy)-4-(2-chloro-4-fluorophenyl)isoquinolin-1(2H)-one, Example 116: To a stirred solution of (R)-2-((4-(2-chloro-4-fluorophenyl)-1-oxo-1,2-dihydroisoquinolin-7-yl)oxy)propanoic acid (Example 90, 100 mg, 0.3 mmol) in CH₂Cl₂ (4 mL) was added DIPEA (0.25 mL, 1.3 mmol) and 1-(piperazin-1-yl)ethan-1-one (55 mg, 0.4 mmol). The reaction mixture was cooled to 0° C., and T₃P (0.25 mL, 0.4 mmol, 50% in EtOAc) was added. The reaction mixture was allowed to warm to ambient temperature and stirred for 16 h. The reaction mixture was quenched with ice cold water and extracted with CH₂Cl₂. The combined organic extracts were washed with brine, dried over anhydrous Na₂SO₄, filtered, and concentrated under reduced pressure. The product was purified by reverse phase prep-HPLC and lyophilized to afford (R)-7-((1-(4-acetylpiperazin-1-yl)-1-oxopropan-2-yl)oxy)-4-(2-chloro-4-fluorophenyl)isoquinolin-1(2H)-one (Example 116, 74 mg). LCMS (ESI) Calcd. for: C₂₄H₂₃ClFN₃O₄: 472, found [M+H]⁺=472. ¹H NMR (400 MHz, DMSO-d₆) δ 11.41 (br s, 1H), 7.62-7.49 (m, 2H), 7.49-7.45 (m, 1H), 7.36-7.34 (m, 1H), 7.34-7.24 (m, 1H), 7.24-7.00 (m, 1H), 6.99-6.93 (m, 1H), 5.49-5.48 (m, 1H), 3.81-3.66 (m, 3H), 3.58-3.54 (m, 3H), 3.31-3.29 (m, 1H), 3.19-3.16 (m, 1H), 2.03 (s, 3H), 1.47-1.45 (d, 3H).

### Example 117: Synthesis of (R)-4-(2-chloro-4-fluorophenyl)-7-((1-oxo-1-(4-propionylpiperazin-1-yl)propan-2-yl)oxy)isoquinolin-1(2H)-one

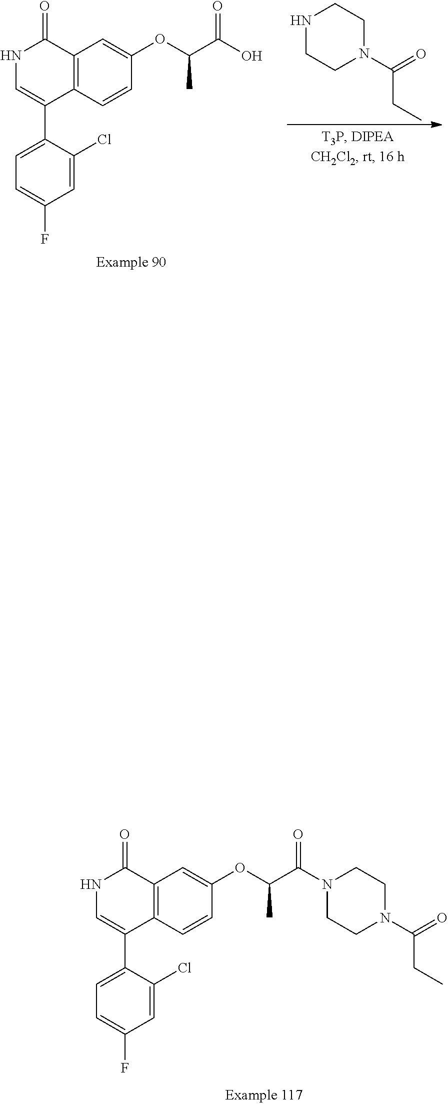

Synthesis of (R)-4-(2-chloro-4-fluorophenyl)-7-((1-oxo-1-(4-propionylpiperazin-1-yl)propan-2-yl)oxy)isoquinolin-1(2H)-one, Example 117: To a stirred solution of (R)-2-((4-(2-chloro-4-fluorophenyl)-1-oxo-1,2-dihydroisoquinolin-7-yl)oxy)propanoic acid (Example 90, 100 mg, 0.3 mmol) in CH₂Cl₂ (3 mL) was added 1-(piperazin-1-yl)propan-1-one (60 mg, 0.4 mmol) and DIPEA (0.15 mL, 0.8 mmol). The reaction mixture was cooled to 0° C., and T₃P (0.5 mL, 0.8 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-oxo-1-(4-propionylpiperazin-1-yl)propan-2-yl)oxy)isoquinolin-1(2H)-one (Example 117, 72 mg). LCMS (ESI) Calcd. for C₂₅H₂₅ClFN₃O₄: 485, found [M+H]⁺=486. ¹H NMR (400 MHz, DMSO-d₆) δ 11.41 (br s, 1H), 7.61-7.60 (m, 2H), 7.49-7.45 (m, 1H), 7.36-7.31 (m, 1H), 7.27-7.24 (m, 1H), 7.00-6.99 (m, 1H), 6.95-6.93 (m, 1H), 5.48 (m, 1H), 3.82-3.70 (m, 3H), 3.59-3.46 (m, 3H), 3.20-3.18 (m, 2H), 2.35-2.33 (m, 2H), 1.46 (d, 3H), 0.99 (t, 3H).

### Example 118: Synthesis of (R)-4-(2-chloro-4-fluorophenyl)-7-((1-oxo-1-(4-(2,2,2-trifluoroethyl)piperazin-1-yl)propan-2-yl)oxy)isoquinolin-1(2H)-one

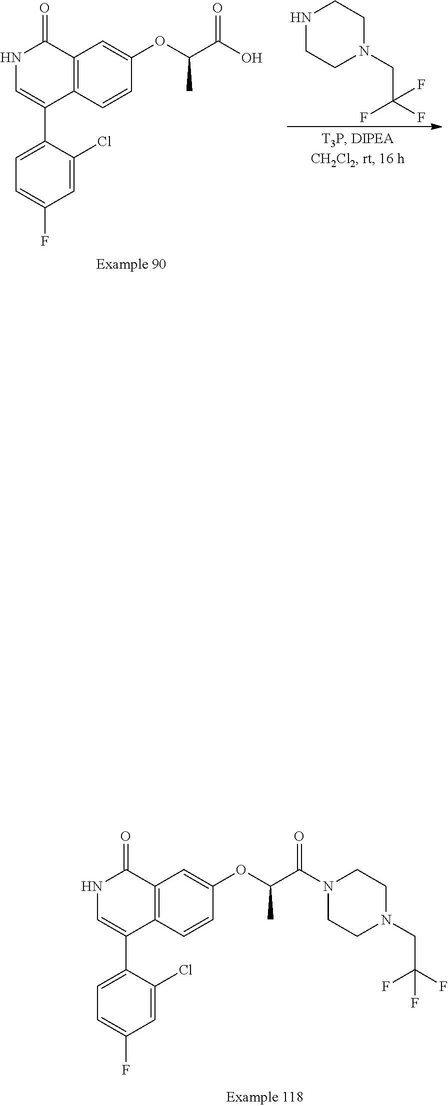 [SMILES: C[C@@H](Oc1ccc2c(-c3ccc(F)cc3Cl)c[nH]c(=O)c2c1)C(=O)O ; FC(F)(F)CN1CCNCC1 ; C[C@@H](Oc1ccc2c(-c3ccc(F)cc3Cl)c[nH]c(=O)c2c1)C(=O)N1CCN(CC(F)(F)F)CC1]

Synthesis of (R)-4-(2-chloro-4-fluorophenyl)-7-((1-oxo-1-(4-(2,2,2-trifluoroethyl)piperazin-1-yl)propan-2-yl)oxy)isoquinolin-1(2H)-one, Example 118: To a stirred solution of (R)-2-((4-(2-chloro-4-fluorophenyl)-1-oxo-1,2-dihydroisoquinolin-7-yl)oxy)propanoic acid (Example 90, 100 mg, 0.3 mmol) in CH₂Cl₂ (3 mL) was added 1-(2,2,2-trifluoroethyl)piperazine (70 mg, 0.4 mmol) and DIPEA (0.15 mL, 0.8 mmol). The reaction mixture was cooled to 0° C., and T₃P (0.5 mL, 0.8 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-oxo-1-(4-(2,2,2-trifluoroethyl)piperazin-1-yl)propan-2-yl)oxy)isoquinolin-1(2H)-one (Example 118, 60 mg). LCMS (ESI) Calcd. for C₂₄H₂₂ClF₄N₃O₃: 511, found [M+H]⁺=512. ¹H NMR (400 MHz, DMSO-d₆) δ 11.45-11.47 (m, 1H), 7.61-7.58 (m, 2H), 7.49-7.45 (m, 1H), 7.36-7.32 (m, 1H), 7.24 (d, 1H), 6.99 (m, 1H), 6.93 (d, 1H), 5.46-5.40 (m, 1H), 3.78 (m, 1H), 3.69-3.66 (m, 1H), 3.54-3.50 (m, 1H), 3.29-3.20 (m, 3H), 2.84-2.82 (m, 1H), 2.71-2.63 (m, 2H), 2.56-2.49 (m, 1H), 1.45 (d, 3H).

### Example 119: Synthesis of 2-methyl-2-((1-oxo-4-(o-tolyl)-1,2-dihydroisoquinolin-7-yl)oxy)propanoic acid

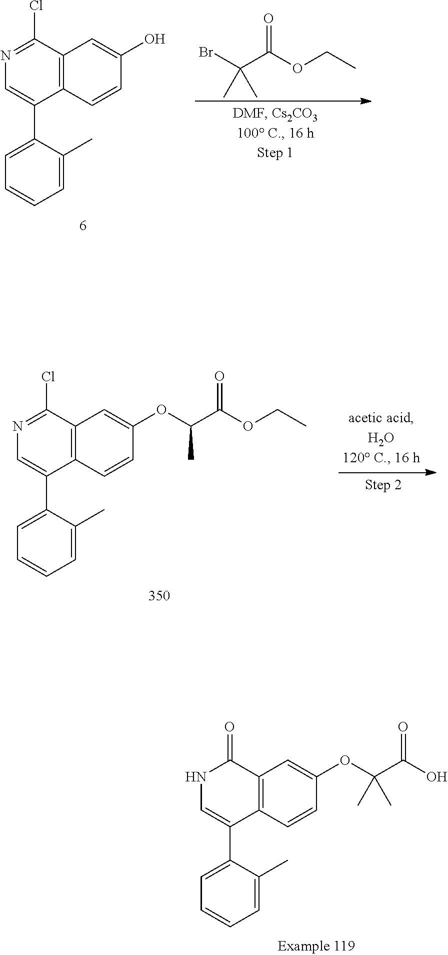

Synthesis of ethyl 2-((1-chloro-4-(o-tolyl)isoquinolin-7-yl)oxy)-2-methylpropanoate, 350 [Step 1]: To a stirred solution of 1-chloro-4-(o-tolyl)isoquinolin-7-ol (6, 150 mg, 0.6 mmol) and ethyl 2-bromo-2-methylpropanoate (220 mg, 1.1 mmol) in DMF (5 mL) was added Cs₂CO₃ (540 mg, 1.7 mmol) at ambient temperature. The reaction mixture was stirred at 100° C. for 16 h. The reaction mixture was quenched with water and extracted with EtOAc. The combined organic extracts were dried over anhydrous Na₂SO₄, filtered, and concentrated under reduced pressure. The product was purified by flash chromatography to afford ethyl 2-((1-chloro-4-(o-tolyl)isoquinolin-7-yl)oxy)-2-methylpropanoate (350, 110 mg). LCMS (ESI) Calcd. for C₂₂H₂₂ClNO₃: 383, found [M+H]⁺=384. ¹H NMR (400 MHz, DMSO-d₆) δ 8.04 (s, 1H), 7.56 (s, 1H), 7.38-7.28 (m, 5H), 7.20 (d, 1H), 4.29 (q, 2H), 2.02 (s, 3H), 1.72 (s, 6H), 1.27 (t, 3H).

Synthesis of 2-methyl-2-((1-oxo-4-(o-tolyl)-1,2-dihydroisoquinolin-7-yl)oxy)propanoic acid, Example 119 [Step 2]: To a stirred solution of ethyl 2-((1-chloro-4-(o-tolyl)isoquinolin-7-yl)oxy)-2-methylpropanoate (350, 100 mg, 0.3 mmol) in acetic acid (2.2 mL, 39.1 mmol) was added water (0.5 mL, 26.1 mmol). The reaction mixture was heated at 120° C. for 16 h. The reaction mixture was concentrated under reduced pressure. The reaction mixture was diluted with EtOAc, washed with water, dried over anhydrous Na₂SO₄, filtered, and concentrated under reduced pressure. The product was purified by reverse phase prep-HPLC to afford 2-methyl-2-((1-oxo-4-(o-tolyl)-1,2-dihydroisoquinolin-7-yl)oxy)propanoic acid (Example 119, 45 mg). LCMS (ESI) Calcd. for C₂₀H₁₉NO₄: 337, found [M+H]⁺=338. ¹H NMR (400 MHz, DMSO-d₆) δ 11.31 (br s, 1H), 7.61 (s, 1H), 7.34-7.33 (m, 2H), 7.29-7.25 (m, 1H), 7.19-7.15 (m, 2H), 6.85 (t, 2H), 2.04 (s, 3H), 1.51 (s, 6H).

### Example 120: Synthesis of (R)-4-((R)-2-((4-(2-chloro-4-fluorophenyl)-1-oxo-1,2-dihydroisoquinolin-7-yl)oxy)propanoyl)morpholine-2-carboxylic acid

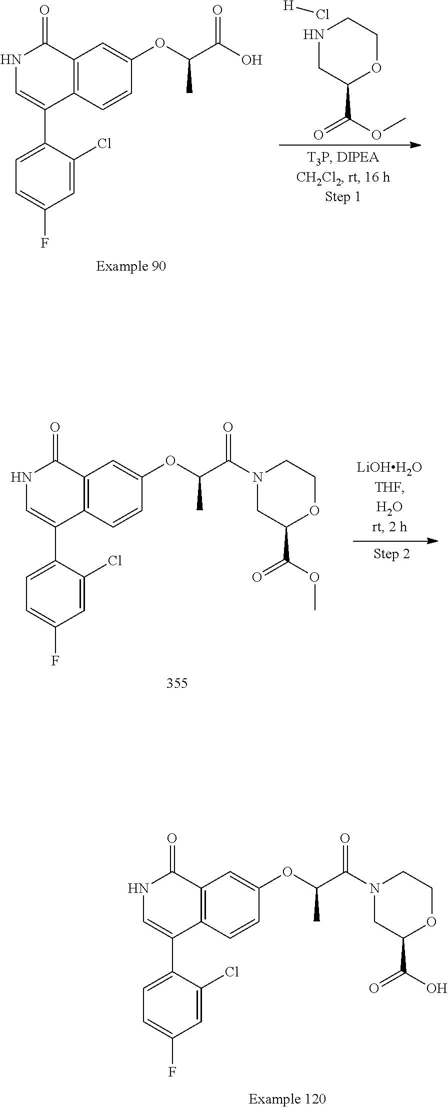

Synthesis of methyl (R)-4-((R)-2-((4-(2-chloro-4-fluorophenyl)-1-oxo-1,2-dihydroisoquinolin-7-yl)oxy)propanoyl)morpholine-2-carboxylate, 355 [Step 1]: To a stirred solution of (R)-2-((4-(2-chloro-4-fluorophenyl)-1-oxo-1,2-dihydroisoquinolin-7-yl)oxy)propanoic acid (Example 90, 100 mg, 0.3 mmol) in CH₂Cl₂ (9 mL) was added DIPEA (0.25 mL, 1.4 mmol) and methyl (R)-morpholine-2-carboxylate hydrochloride (75 mg, 0.4 mmol). The reaction mixture was cooled to 0° C., and T₃P (0.25 mL, 0.4 mmol, 50% in EtOAc) was added. The reaction mixture was allowed to warm to ambient temperature and stirred for 16 h. The reaction mixture was quenched with water and extracted with CH₂Cl₂. The combined organic extracts were washed with brine, dried over anhydrous Na₂SO₄, filtered, and concentrated under reduced pressure. The product was purified by flash chromatography to afford methyl (R)-4-((R)-2-((4-(2-chloro-4-fluorophenyl)-1-oxo-1,2-dihydroisoquinolin-7-yl)oxy)propanoyl)morpholine-2-carboxylate (355, 100 mg). LCMS (ESI) Calcd. for C₂₄H₂₂ClFN₂O₆: 488, found [M+H]+=489.

Synthesis of (R)-4-((R)-2-((4-(2-chloro-4-fluorophenyl)-1-oxo-1,2-dihydroisoquinolin-7-yl)oxy)propanoyl)morpholine-2-carboxylic acid, Example 120 [Step 2]: To a stirred solution of (R)-4-((R)-2-((4-(2-chloro-4-fluorophenyl)-1-oxo-1,2-dihydroisoquinolin-7-yl)oxy)propanoyl)morpholine-2-carboxylate (355, 160 mg, 0.3 mmol) in THF (8 mL) was added an aq. solution (2 mL) of LiOH·H₂O (50 mg, 1.1 mmol) at ambient temperature. The reaction mixture was stirred for 2 h. The reaction mixture was concentrated under reduced pressure. The reaction mixture was diluted with water, acidified with citric acid, and extracted with EtOAc (×3). The combined organic extracts were washed with brine, dried over anhydrous Na₂SO₄, and concentrated under reduced pressure. The product was purified by reverse phase prep-HPLC and lyophilized to afford (R)-4-((R)-2-((4-(2-chloro-4-fluorophenyl)-1-oxo-1,2-dihydroisoquinolin-7-yl)oxy)propanoyl)morpholine-2-carboxylic acid (Example 120, 52 mg). LCMS (ESI) Calcd. for C₂₃H₂₀ClFN₂O₆: 474, found [M+H]⁺=475. ¹H NMR (400 MHz, DMSO-d₆) δ 11.43 (br s, 1H), 7.61-7.60 (m, 2H), 7.50-7.46 (m, 1H), 7.34 (t, 1H), 7.25 (t, 1H), 7.00 (s, 1H), 6.94 (d, 1H), 5.48-5.40 (m, 1H), 4.32-3.85 (m, 5H), 3.53-2.66 (m, 2H), 1.46 (d, 3H).

### Example 121: Synthesis of (S)-4-((R)-2-((4-(2-chloro-4-fluorophenyl)-1-oxo-1,2-dihydroisoquinolin-7-yl)oxy)propanoyl)morpholine-2-carboxylic acid

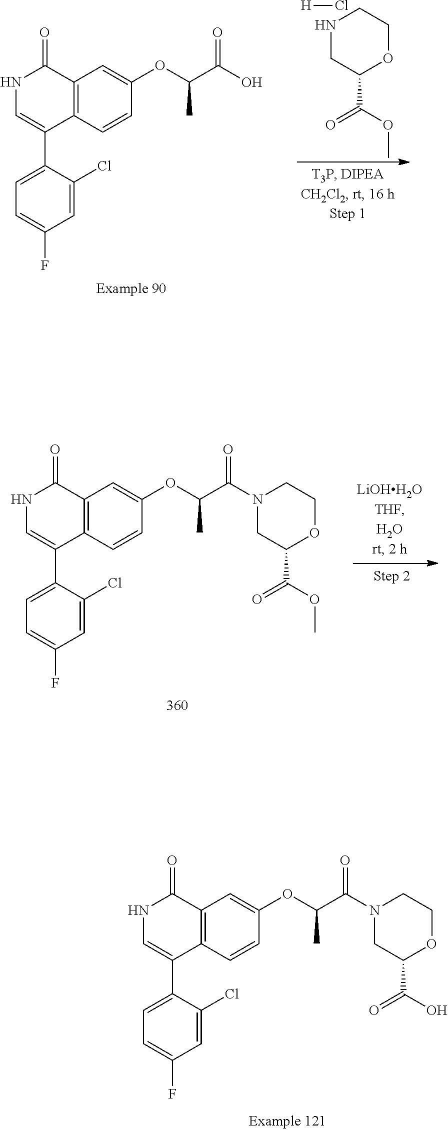

Synthesis of methyl (S)-4-((R)-2-((4-(2-chloro-4-fluorophenyl)-1-oxo-1,2-dihydroisoquinolin-7-yl)oxy)propanoyl)morpholine-2-carboxylate, 360 [Step 1]: To a stirred solution of (R)-2-((4-(2-chloro-4-fluorophenyl)-1-oxo-1,2-dihydroisoquinolin-7-yl)oxy)propanoic acid (Example 90, 100 mg, 0.3 mmol) in CH₂Cl₂ (4 mL) was added DIPEA (0.25 mL, 1.4 mmol) and methyl (S)-morpholine-2-carboxylate hydrochloride (76 mg, 0.4 mmol). The reaction mixture was cooled to 0° C., and T₃P (0.25 mL, 0.4 mmol, 50% in EtOAc) was added. The reaction mixture was allowed to warm to ambient temperature and stirred for 16 h. The reaction mixture was quenched with ice cold water and extracted with CH₂Cl₂. The organic extract was washed with brine, dried over anhydrous Na₂SO₄, filtered, and concentrated under reduced pressure. The product was purified by flash chromatography to afford methyl (S)-4-((R)-2-((4-(2-chloro-4-fluorophenyl)-1-oxo-1,2-dihydroisoquinolin-7-yl)oxy)propanoyl)morpholine-2-carboxylate (360, 130 mg). LCMS (ESI) Calcd. for C₂₄H₂₂ClFN₂O₆: 488, found [M+H]+=489.

Synthesis of (S)-4-((R)-2-((4-(2-chloro-4-fluorophenyl)-1-oxo-1,2-dihydroisoquinolin-7-yl)oxy)propanoyl)morpholine-2-carboxylic acid, Example 121 [Step 2]: To a stirred solution of methyl (S)-4-((R)-2-((4-(2-chloro-4-fluorophenyl)-1-oxo-1,2-dihydroisoquinolin-7-yl)oxy)propanoyl)morpholine-2-carboxylate (360, 80 mg, 0.2 mmol) in THF (4 mL) was added an aq. solution (1 mL) of LiOH·H₂O (25 mg, 0.6 mmol) at ambient temperature. The reaction mixture was stirred for 2 h. The reaction mixture was concentrated under reduced pressure. The reaction mixture was diluted with water, acidified with 1M citric acid to pH=5, and extracted with EtOAc (×3). The combined organic extracts were washed with brine, dried over anhydrous Na₂SO₄, filtered, and concentrated under reduced pressure. The product was purified by reverse phase prep-HPLC and lyophilized to afford (S)-4-((R)-2-((4-(2-chloro-4-fluorophenyl)-1-oxo-1,2-dihydroisoquinolin-7-yl)oxy)propanoyl)morpholine-2-carboxylic acid (Example 121, 20 mg). LCMS (ESI) Calcd. for C₂₃H₂₀ClFN₂O₆: 474, found [M+H]⁺=475. ¹H NMR (400 MHz, DMSO-d₆) δ 13.30 (br s, 1H), 11.40 (br s, 1H), 7.61-7.60 (m, 2H), 7.50-7.46 (m, 1H), 7.36-7.32 (m, 1H), 7.27-7.24 (m, 1H), 7.00 (br s, 1H), 6.96-6.93 (m, 1H), 5.49-5.44 (m, 1H), 4.30-4.27 (m, 1H), 3.97-3.95 (m, 2H), 3.85-3.83 (m, 1H), 3.74-3.49 (m, 1H), 3.14-2.92 (m, 2H), 1.50-1.48 (d, 3H).

### Example 122: Synthesis of (R)-4-((R)-2-((4-(2-chloro-4-fluorophenyl)-1-oxo-1,2-dihydroisoquinolin-7-yl)oxy)propanoyl)morpholine-3-carboxamide

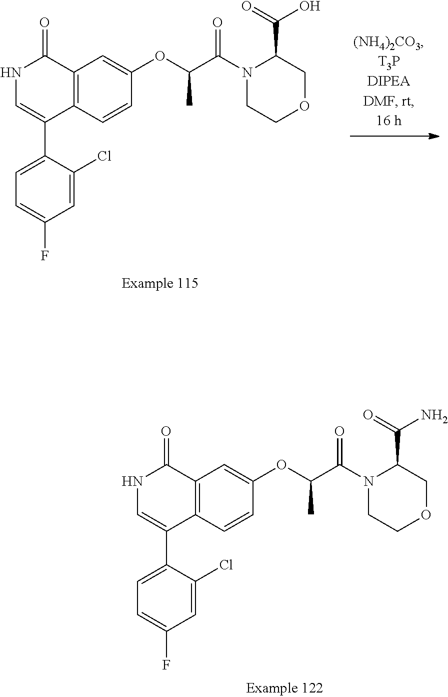 [SMILES: C[C@@H](Oc1ccc2c(-c3ccc(F)cc3Cl)c[nH]c(=O)c2c1)C(=O)N1CCOC[C@@H]1C(=O)O ; C[C@@H](Oc1ccc2c(-c3ccc(F)cc3Cl)c[nH]c(=O)c2c1)C(=O)N1CCOC[C@@H]1C(N)=O]

Synthesis of (R)-4-((R)-2-((4-(2-chloro-4-fluorophenyl)-1-oxo-1,2-dihydroisoquinolin-7-yl)oxy)propanoyl)morpholine-3-carboxamide, Example 122: To a stirred solution of (R)-4-((R)-2-((4-(2-chloro-4-fluorophenyl)-1-oxo-1,2-dihydroisoquinolin-7-yl)oxy)propanoyl)morpholine-3-carboxylic acid (Example 115, 110 mg, 0.2 mmol) in DMF (5 mL) was added (NH₄)₂CO₃ (110 mg, 1.2 mmol) and DIPEA (0.12 mL, 0.7 mmol). The reaction mixture was cooled to 0° C., and T₃P (0.3 mL, 0.5 mmol, 50% in EtOAc) was added. The reaction mixture was allowed to warm to ambient temperature and stirred for 16 h. The reaction mixture was diluted with EtOAc, washed with water, brine, dried over anhydrous Na₂SO₄, filtered, and concentrated under reduced pressure. The product was purified by reverse phase prep-HPLC and lyophilized to afford (R)-4-((R)-2-((4-(2-chloro-4-fluorophenyl)-1-oxo-1,2-dihydroisoquinolin-7-yl)oxy)propanoyl)morpholine-3-carboxamide (Example 122, 30 mg). LCMS (ESI) Calcd. for C₂₃H₂₁ClFN₃O₅: 473, found [M+H]⁺=474. ¹H NMR (400 MHz, DMSO-d₆) δ 11.42-11.41 (m, 1H), 7.71-7.70 (m, 2H), 7.62-7.50 (m, 1H), 7.48-7.20 (m, 3H), 7.00-6.89 (m, 3H), 5.55-5.53 (m, 1H), 4.64-4.63 (m, 1H), 4.29-4.26 (m, 1H), 3.94-3.79 (m, 2H), 3.66-3.55 (m, 3H), 1.46-1.44 (m, 3H).

### Example 123: Synthesis of (S)-4-((R)-2-((4-(2-chloro-4-fluorophenyl)-1-oxo-1,2-dihydroisoquinolin-7-yl)oxy)propanoyl)morpholine-3-carboxamide

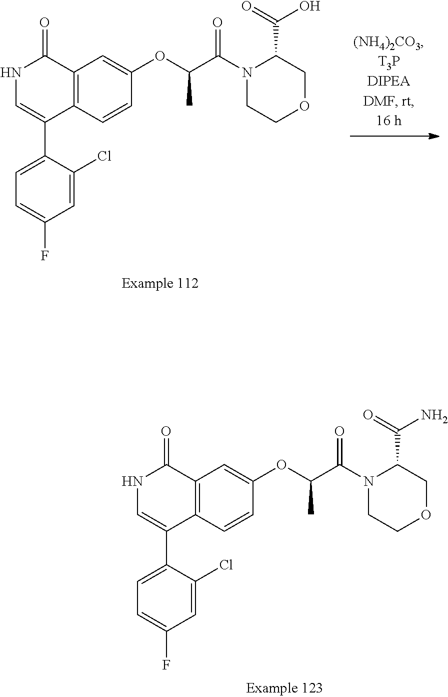 [SMILES: C[C@@H](Oc1ccc2c(-c3ccc(F)cc3Cl)c[nH]c(=O)c2c1)C(=O)N1CCOC[C@H]1C(=O)O ; C[C@@H](Oc1ccc2c(-c3ccc(F)cc3Cl)c[nH]c(=O)c2c1)C(=O)N1CCOC[C@H]1C(N)=O]

Synthesis of (S)-4-((R)-2-((4-(2-chloro-4-fluorophenyl)-1-oxo-1,2-dihydroisoquinolin-7-yl)oxy)propanoyl)morpholine-3-carboxamide, Example 123: To a stirred solution of (S)-4-((R)-2-((4-(2-chloro-4-fluorophenyl)-1-oxo-1,2-dihydroisoquinolin-7-yl)oxy)propanoyl)morpholine-3-carboxylic acid (Example 112, 80 mg, 0.2 mmol) in DMF (3 mL) was added (NH₄)₂CO₃ (160 mg, 1.7 mmol) and DIPEA (0.15 mL, 0.8 mmol). The reaction mixture was cooled to 0° C., and T₃P (0.15 mL, 0.3 mmol) was added. The reaction mixture was allowed to warm to ambient temperature and stirred for 16 h. The reaction mixture was diluted with ice cold water and extracted with EtOAc (×3). The combined organic extracts were washed with brine, dried over anhydrous Na₂SO₄, filtered, and concentrated under reduced pressure. The product was purified by reverse phase prep-HPLC and lyophilized to afford (S)-4-((R)-2-((4-(2-chloro-4-fluorophenyl)-1-oxo-1,2-dihydroisoquinolin-7-yl)oxy)propanoyl)morpholine-3-carboxamide (Example 123, 17 mg). LCMS (ESI) Calcd. for C₂₃H₂₁ClFN₃O₅: 473, found [M+H]⁺=474. ¹H NMR (400 MHz, DMSO-d₆) δ 11.41 (br s, 1H), 7.71-7.70 (m, 2H), 7.62-7.50 (m, 1H), 7.48-7.20 (m, 3H), 7.00-6.89 (m, 3H), 5.46-5.32 (m, 1H), 4.85-3.94 (m, 3H), 3.82-2.96 (m, 4H), 1.51-1.39 (m, 3H).

### Example 124-125: Synthesis of chiral-4-(2-chloro-4-fluorophenyl)-7-(((R)-1-(trans-2,6-dimethylmorpholino)-1-oxopropan-2-yl)oxy)isoquinolin-1(2H)-one

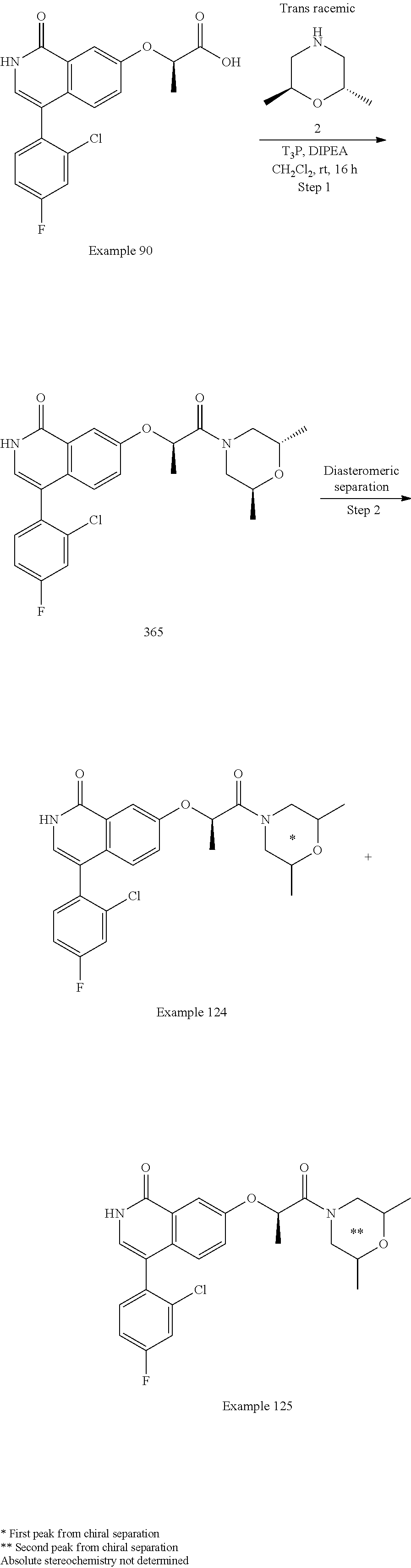

Synthesis of 4-(2-chloro-4-fluorophenyl)-7-(((2R)-1-(trans-rac-2,6-dimethylmorpholino)-1-oxopropan-2-yl)oxy)isoquinolin-1(2H)-one, 365 [Step 1]: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (9 mL) was added DIPEA (0.19 mL, 1.1 mmol) and trans-2,6-dimethylmorpholine (40 mg, 0.3 mmol). The reaction mixture was cooled to 0° C., and T₃P (0.2 mL, 0.3 mmol, 50% in EtOAc) was added. The reaction mixture was allowed to warm to ambient temperature and stirred for 16 h. The reaction mixture was quenched with water and extracted with CH₂Cl₂. The combined organic extracts were washed with brine, dried over anhydrous Na₂SO₄, filtered, and concentrated under reduced pressure. The product was purified by reverse phase prep-HPLC and lyophilized to afford 4-(2-chloro-4-fluorophenyl)-7-(((2R)-1-(trans-rac-2,6-dimethylmorpholino)-1-oxopropan-2-yl)oxy)isoquinolin-1(2H)-one (365, 50 mg). LCMS (ESI) Calcd. for C₂₄H₂₄ClFN₂O₄: 458, found [M+H]⁺=459.

Synthesis of chiral-4-(2-chloro-4-fluorophenyl)-7-(((R)-1-(trans-2,6-dimethylmorpholino)-1-oxopropan-2-yl)oxy)isoquinolin-1(2H)-one, Example 124 and Example 125 [Step 2]: The diastereomeric mixture of 4-(2-chloro-4-fluorophenyl)-7-(((2R)-1-(trans-rac-2,6-dimethylmorpholino)-1-oxopropan-2-yl)oxy)isoquinolin-1(2H)-one (365, 50 mg, 0.1 mmol) was separated by normal phase chiral prep-HPLC and lyophilized. The first product was isolated as 4-(2-chloro-4-fluorophenyl)-7-(((2R)-1-(trans-2,6-dimethylmorpholino)-1-oxopropan-2-yl)oxy)isoquinolin-1(2H)-one, Peak 1 (Example 124, 23 mg) and the second product as 4-(2-chloro-4-fluorophenyl)-7-(((2R)-1-(trans-2,6-dimethylmorpholino)-1-oxopropan-2-yl)oxy)isoquinolin-1(2H)-one, Peak 2 (Example 125, 22 mg). The absolute stereochemistry of these Examples was not determined.

Example 124: 4-(2-chloro-4-fluorophenyl)-7-(((2R)-1-(trans-2,6-dimethylmorpholino)-1-oxopropan-2-yl)oxy)isoquinolin-1(2H)-one, Peak 1: LCMS (ESI) Calcd. for C₂₄H₂₄ClFN₂O₄: 458, found [M−H]⁻=457. ¹H NMR (400 MHz, DMSO-d₆) δ 11.39 (br s, 1H), 7.61-7.60 (m, 2H), 7.48 (t, 1H), 7.36-7.32 (m, 1H), 7.28-7.24 (m, 1H), 6.99 (s, 1H), 6.94 (d, 1H), 5.55-5.52 (m, 1H), 4.14-4.11 (m, 1H), 3.97-3.95 (m, 2H), 3.62-3.59 (m, 1H), 3.16-3.04 (m, 2H), 1.45-1.44 (m, 3H), 1.09-1.08 (m, 6H).

Example 125: 4-(2-chloro-4-fluorophenyl)-7-(((2R)-1-(trans-2,6-dimethylmorpholino)-1-oxopropan-2-yl)oxy)isoquinolin-1(2H)-one, Peak 2: LCMS (ESI) Calcd. for C₂₄H₂₄ClFN₂O₄: 458, found [M−H]⁻=457. ¹H NMR (400 MHz, DMSO-d₆) δ 11.40 (br s, 1H), 7.60-7.59 (m, 2H), 7.50-7.45 (m, 1H), 7.36-7.32 (m, 1H), 7.26-7.23 (m, 1H), 6.99 (s, 1H), 6.94 (d, 1H), 5.41-5.38 (m, 1H), 3.91-3.11 (m, 6H), 1.48-1.46 (m, 3H), 1.22-1.00 (m, 6H).

Chiral prep-HPLC: Diastereomeric separation was performed on an Agilent 1200 series instrument. Column was a Chiralcel OD-H (250×20 mm), 5 μm, operating at ambient temperature with flow rate of 18.0 mL/min. Mobile phase: 0.1% isopropylamine in a mixture of 90% hexanes, 10% ethanol, held isocratic for up to 23 min. with detection at 282 nm wavelength.

### Example 126: Synthesis of (R)-2-((1-oxo-4-(o-tolyl)-1,2-dihydroisoquinolin-7-yl)oxy)butanoic acid

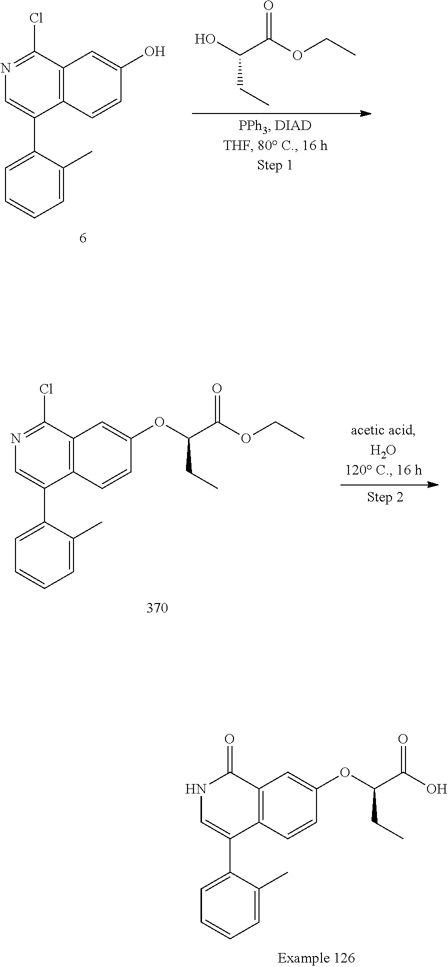

Synthesis of ethyl (R)-2-((1-chloro-4-(o-tolyl)isoquinolin-7-yl)oxy)butanoate, 370 [Step 1]: To a stirred solution of 1-chloro-4-(o-tolyl)isoquinolin-7-ol (6, 170 mg, 0.6 mmol) and ethyl (S)-2-hydroxybutanoate (125 mg, 0.9 mmol) in THF (10 mL) was added PPh₃ (490 mg, 1.9 mmol). The reaction mixture was cooled to 0° C., and DIAD (0.4 mL, 1.9 mmol) was added dropwise. The reaction mixture was stirred for 5 min. at 0° C. and then at 80° C. for 16 h. The reaction mixture was diluted with EtOAc, washed with water, brine, dried over anhydrous Na₂SO₄, filtered, and concentrated under reduced pressure. The product was purified by column chromatography to afford ethyl (R)-2-((1-chloro-4-(o-tolyl)isoquinolin-7-yl)oxy)butanoate (370, 165 mg). LCMS (ESI) Calcd. for C₂₂H₂₂ClNO₃: 383, found [M+H]⁺=384.

Synthesis of (R)-2-((1-oxo-4-(o-tolyl)-1,2-dihydroisoquinolin-7-yl)oxy)butanoic acid, Example 126 [Step 2]: To a stirred solution of ethyl (R)-2-((1-chloro-4-(o-tolyl)isoquinolin-7-yl)oxy)butanoate (370, 160 mg, 0.4 mmol) in acetic acid (3.6 mL, 62.5 mmol) was added water (0.8 mL, 41.7 mmol). The reaction mixture was heated at 120° C. for 16 h. The reaction mixture was concentrated under reduced pressure. The reaction mixture was diluted with EtOAc, washed with water, dried over anhydrous Na₂SO₄, filtered, and concentrated under reduced pressure. The product was purified by reverse phase prep-HPLC to afford (R)-2-((1-oxo-4-(o-tolyl)-1,2-dihydroisoquinolin-7-yl)oxy)butanoic acid (Example 126, 90 mg). LCMS (ESI) Calcd. for C₂₀H₁₉NO₄: 337, found [M+H]⁺=338. ¹H NMR (400 MHz, DMSO-d₆) δ 11.33 (br s, 1H), 7.58 (s, 1H), 7.35-7.33 (m, 2H), 7.29-7.22 (m, 2H), 7.18 (d, 1H), 6.89-6.86 (m, 2H), 4.57 (br s, 1H), 2.04 (s, 3H), 1.95-1.81 (m, 2H), 0.99 (t, 3H).

### Example 127: Synthesis of 4-(2-chloro-4-fluorophenyl)-7-(((2R)-1-(5-methyl-2,5-diazabicyclo[2.2.1]heptan-2-yl)-1-oxopropan-2-yl)oxy)isoquinolin-1(2H)-one

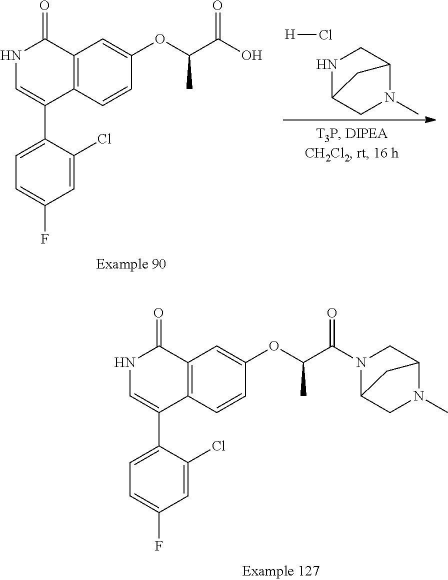

Synthesis of 4-(2-chloro-4-fluorophenyl)-7-(((2R)-1-(5-methyl-2,5-diazabicyclo[2.2.1]heptan-2-yl)-1-oxopropan-2-yl)oxy)isoquinolin-1(2H)-one, Example 127: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (3 mL) was added 2-methyl-2,5-diazabicyclo[2.2.1]heptane hydrochloride (60 mg, 0.3 mmol) and DIPEA (0.1 mL, 0.7 mmol). The reaction mixture was cooled to 0° C., and T₃P (0.4 mL, 0.7 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure. The product was purified by reverse phase prep-HPLC and lyophilized to afford 4-(2-chloro-4-fluorophenyl)-7-(((2R)-1-(5-methyl-2,5-diazabicyclo[2.2.1]heptan-2-yl)-1-oxopropan-2-yl)oxy)isoquinolin-1(2H)-one (Example 127, 60 mg). LCMS (ESI) Calcd. for C₂₄H₂₃ClFN₃O₃: 455, found [M+H]⁺=456. ¹H NMR (400 MHz, DMSO-d₆) δ 11.41 (br s, 1H), 7.61-7.59 (m, 2H), 7.56-7.46 (m, 1H), 7.36-7.22 (m, 2H), 7.00-6.93 (m, 2H), 5.26-4.48 (m, 2H), 3.87-2.84 (m, 5H), 2.24-2.21 (m, 3H), 1.85-1.56 (m, 2H), 1.48-1.46 (m, 3H). The compound was isolated as mixture of diastereomers.

### Example 128: Synthesis of (R)-4-(2-chloro-4-fluorophenyl)-7-((1-(1-oxidothiomorpholino)-1-oxopropan-2-yl)oxy)isoquinolin-1(2H)-one

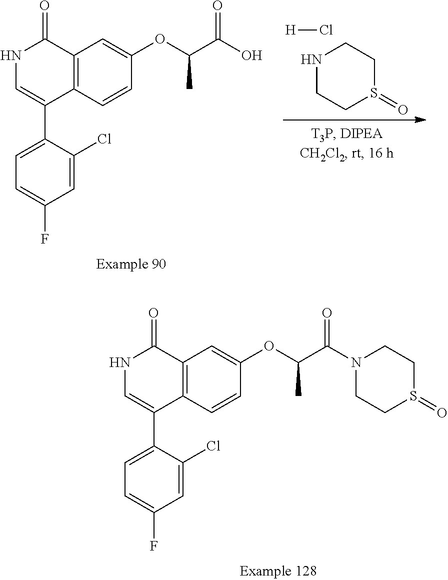

Synthesis of (R)-4-(2-chloro-4-fluorophenyl)-7-((1-(1-oxidothiomorpholino)-1-oxopropan-2-yl)oxy)isoquinolin-1(2H)-one, Example 128: To a stirred solution of (R)-2-((4-(2-chloro-4-fluorophenyl)-1-oxo-1,2-dihydroisoquinolin-7-yl)oxy)propanoic acid (Example 90, 100 mg, 0.3 mmol) in CH₂Cl₂ (3 mL) was added DIPEA (0.15 mL, 0.8 mmol) and T₃P (0.5 mL, 0.8 mmol, 50% in EtOAc). Thiomorpholine 1-oxide hydrochloride (45 mg, 0.3 mmol) was added at 0° C. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-(1-oxidothiomorpholino)-1-oxopropan-2-yl)oxy)isoquinolin-1(2H)-one (Example 128, 30 mg). LCMS (ESI) Calcd. for C₂₂H₂₀ClFN₂O₄S: 462, found [M+H]⁺=463. ¹H NMR (400 MHz, DMSO-d₆) δ 11.46 (br s, 1H), 7.62-7.53 (m, 2H), 7.49-7.45 (m, 1H), 7.36-7.32 (m, 1H), 7.28-7.26 (m, 1H), 7.01-7.00 (m, 1H), 6.96-6.94 (m, 1H), 5.53-5.52 (m, 1H), 4.36-4.32 (m, 1H), 4.09-3.95 (m, 3H), 3.53-3.46 (m, 1H), 2.88-2.66 (m, 3H), 1.48-1.47 (m, 3H).

### Example 129: Synthesis of (R)-4-((R)-2-((4-(2-chloro-4-fluorophenyl)-1-oxo-1,2-dihydroisoquinolin-7-yl)oxy)propanoyl)morpholine-2-carboxamide

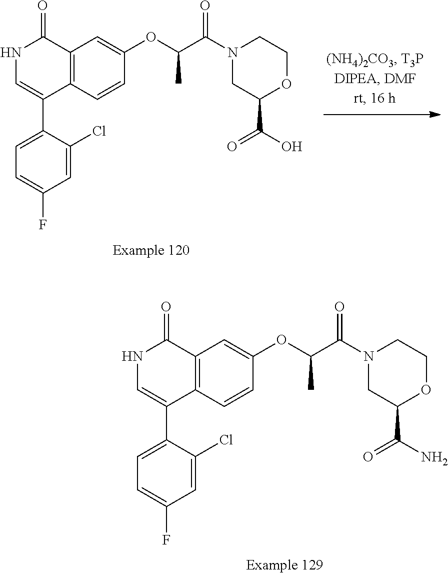 [SMILES: C[C@@H](Oc1ccc2c(-c3ccc(F)cc3Cl)c[nH]c(=O)c2c1)C(=O)N1CCO[C@@H](C(=O)O)C1 ; C[C@@H](Oc1ccc2c(-c3ccc(F)cc3Cl)c[nH]c(=O)c2c1)C(=O)N1CCO[C@@H](C(N)=O)C1]

Synthesis of (R)-4-((R)-2-((4-(2-chloro-4-fluorophenyl)-1-oxo-1,2-dihydroisoquinolin-7-yl)oxy)propanoyl)morpholine-2-carboxamide, Example 129: To a stirred solution of (R)-4-((R)-2-((4-(2-chloro-4-fluorophenyl)-1-oxo-1,2-dihydroisoquinolin-7-yl)oxy)propanoyl)morpholine-2-carboxylic acid (Example 120, 110 mg, 0.2 mmol) in CH₂Cl₂ (9 mL) was added (NH₄)₂CO₃ (225 mg, 2.3 mmol) and DIPEA (0.2 mL, 1.2 mmol). The reaction mixture was cooled to 0° C., and T₃P (0.2 mL, 0.5 mmol, 50% in EtOAc) was added. The reaction mixture was allowed to warm to ambient temperature and stirred for 16 h. The reaction mixture was quenched with water and extracted with CH₂Cl₂ (×3). The combined organic extracts were washed with brine, dried over anhydrous Na₂SO₄, filtered, and concentrated under reduced pressure. The product was purified by reverse phase prep-HPLC and lyophilized to afford (R)-4-((R)-2-((4-(2-chloro-4-fluorophenyl)-1-oxo-1,2-dihydroisoquinolin-7-yl)oxy)propanoyl)morpholine-2-carboxamide (Example 129, 53 mg). LCMS (ESI) Calcd. for C₂₃H₂₁ClFN₃O₅: 473, found [M−H]⁻=472. ¹H NMR (400 MHz, DMSO-d₆) at 100° C. δ 11.08 (br s, 1H), 7.71 (s, 1H), 7.52-7.45 (m, 2H), 7.32-7.25 (m, 2H), 7.00-6.94 (m, 4H), 5.38-5.34 (m, 1H), 4.29 (br s, 1H), 4.03-3.93 (m, 3H), 3.56-3.51 (m, 1H), 3.12 (br s, 2H), 1.53 (d, 3H).

### Example 130: Synthesis of (R)-4-(2-chloro-4-fluorophenyl)-7-((1-(4-(methylsulfonyl)piperazin-1-yl)-1-oxopropan-2-yl)oxy)isoquinolin-1(2H)-one

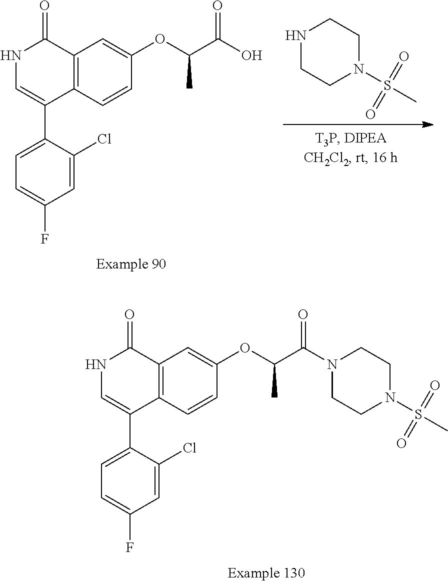

Synthesis of (R)-4-(2-chloro-4-fluorophenyl)-7-((1-(4-(methylsulfonyl)piperazin-1-yl)-1-oxopropan-2-yl)oxy)isoquinolin-1(2H)-one, Example 130: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (3 mL) was added 1-methylsulfonylpiperazine (55 mg, 0.3 mmol) and DIPEA (0.1 mL, 0.7 mmol). The reaction mixture was cooled to 0° C., and T₃P (0.4 mL, 0.7 mmol, 50% in EtOAc) was added. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-(4-(methylsulfonyl)piperazin-1-yl)-1-oxopropan-2-yl)oxy)isoquinolin-1(2H)-one (Example 130, 46 mg). LCMS (ESI) Calcd. for C₂₃H₂₃ClFN₃O₅S: 507, found [M+H]⁺=508. ¹H NMR (400 MHz, DMSO-d₆) δ 11.47 (br s, 1H), 7.62-7.58 (m, 2H), 7.49-7.44 (m, 1H), 7.36-7.32 (m, 1H), 7.28-7.26 (m, 1H), 6.99-6.94 (m, 2H), 5.52-5.51 (m, 1H), 4.07-3.92 (m, 2H), 3.58-3.56 (m, 1H), 3.30-3.24 (m, 5H), 2.97-2.96 (m, 3H), 1.48-1.46 (m, 3H).

### Example 131: Synthesis of (R)-4-(2-chloro-4-fluorophenyl)-7-((1-(4-methylpiperazin-1-yl)-1-oxopropan-2-yl)oxy)isoquinolin-1(2H)-one

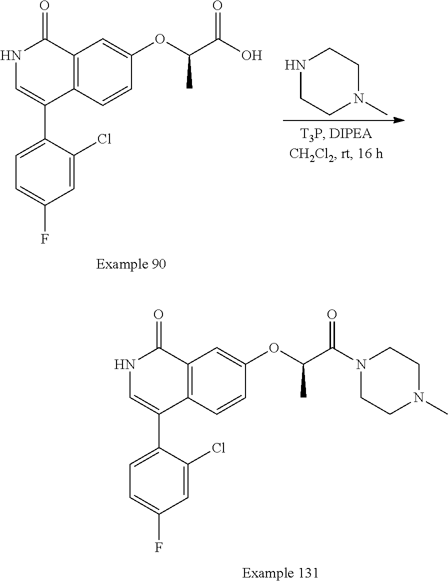

Synthesis of (R)-4-(2-chloro-4-fluorophenyl)-7-((1-(4-methylpiperazin-1-yl)-1-oxopropan-2-yl)oxy)isoquinolin-1(2H)-one, Example 131: To a stirred solution of (R)-2-((4-(2-chloro-4-fluorophenyl)-1-oxo-1,2-dihydroisoquinolin-7-yl)oxy)propanoic acid (Example 90, 80 mg, 0.2 mmol) in CH₂Cl₂ (3 mL) was added 1-methylpiperazine (33 mg, 0.3 mmol) and DIPEA (0.1 mL, 0.7 mmol). T₃P (0.4 mL, 0.7 mmol, 50% in EtOAc) was added to the reaction mixture at 0° C. The reaction mixture was allowed to warm to ambient temperature and stirred for 16 h. The reaction mixture was diluted with CH₂Cl₂, washed with water, brine, dried over anhydrous Na₂SO₄, filtered, and concentrated under reduced pressure. The product was purified by reverse phase prep-HPLC and lyophilized to afford (R)-4-(2-chloro-4-fluorophenyl)-7-((1-(4-methylpiperazin-1-yl)-1-oxopropan-2-yl)oxy)isoquinolin-1(2H)-one (Example 131, 53 mg). LCMS (ESI) Calcd. for C₂₃H₂₃ClFN₃O₃: 444, found [M+H]⁺=444. ¹H NMR (400 MHz, DMSO-d₆) δ 11.42 (br s, 1H), 7.62-7.59 (m, 2H), 7.49-7.45 (m, 1H), 7.36-7.31 (m, 1H), 7.25-7.22 (m, 1H), 6.99-6.92 (m, 2H), 5.45-5.39 (m, 1H), 3.73-3.23 (m, 4H), 2.49-2.23 (m, 4H), 2.20 (s, 3H), 1.45-1.44 (m, 3H).

### Example 132: Synthesis of (S)-4-((R)-2-((4-(2-chloro-4-fluorophenyl)-1-oxo-1,2-dihydroisoquinolin-7-yl)oxy)propanoyl)morpholine-2-carboxamide

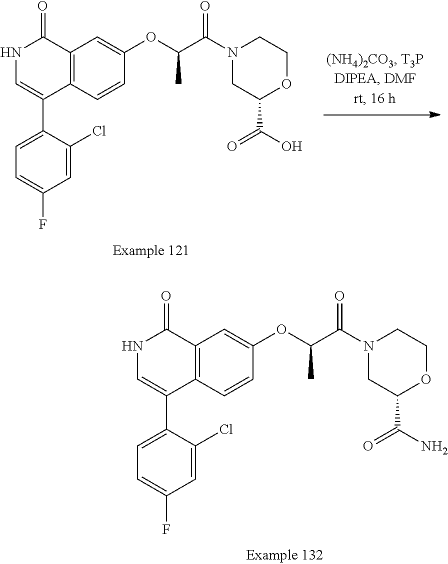 [SMILES: C[C@@H](Oc1ccc2c(-c3ccc(F)cc3Cl)c[nH]c(=O)c2c1)C(=O)N1CCO[C@H](C(=O)O)C1 ; C[C@@H](Oc1ccc2c(-c3ccc(F)cc3Cl)c[nH]c(=O)c2c1)C(=O)N1CCO[C@H](C(N)=O)C1]

Synthesis of (S)-4-((R)-2-((4-(2-chloro-4-fluorophenyl)-1-oxo-1,2-dihydroisoquinolin-7-yl)oxy)propanoyl)morpholine-2-carboxamide, Example 132: To a stirred solution of (S)-4-((R)-2-((4-(2-chloro-4-fluorophenyl)-1-oxo-1,2-dihydroisoquinolin-7-yl)oxy)propanoyl)morpholine-2-carboxylic acid (Example 121, 180 mg, 0.4 mmol) in DMF (4 mL) was added DIPEA (0.33 mL, 1.9 mmol) and (NH₄)₂CO₃ (180 mg, 1.9 mmol). T₃P (0.4 mL, 0.6 mmol, 50% in EtOAc) was added to the reaction mixture at 0° C. The reaction mixture was allowed to warm to ambient temperature and stirred for 16 h. The reaction mixture was quenched with ice cold water, extracted with EtOAc (×3). The combined organic extracts were washed with brine, dried over anhydrous Na₂SO₄, filtered, and concentrated under reduced pressure. The product was purified by reverse phase prep-HPLC and lyophilized to afford (S)-4-((R)-2-((4-(2-chloro-4-fluorophenyl)-1-oxo-1,2-dihydroisoquinolin-7-yl)oxy)propanoyl)morpholine-2-carboxamide (Example 132, 40 mg). LCMS (ESI) Calcd. for C₂₃H₂₁ClFN₃O₅: 474, found [M+H]⁺=474. ¹H NMR (400 MHz, DMSO-d₆) δ 11.40 (br s, 1H), 7.61 (br s, 2H), 7.50-7.46 (m, 1H), 7.36-7.32 (m, 2H), 7.27-7.25 (m, 2H), 7.00-6.94 (m, 2H), 5.52-5.51 (m, 1H), 4.47-4.43 (m, 1H), 4.12-4.10 (m, 1H), 4.00-3.97 (m, 1H), 3.90-3.87 (m, 1H), 3.84-3.80 (m, 1H), 2.95 (br s, 1H), 2.66-2.63 (m, 1H), 1.51-1.44 (m, 3H).

### Example 133-134: Synthesis of 2-((4-(2-methylthiophen-3-yl)-1-oxo-1,2-dihydroisoquinolin-7-yl)oxy)acetonitrile and 2-((4-(2-methylthiophen-3-yl)-1-oxo-1,2-dihydroisoquinolin-7-yl)oxy)acetamide

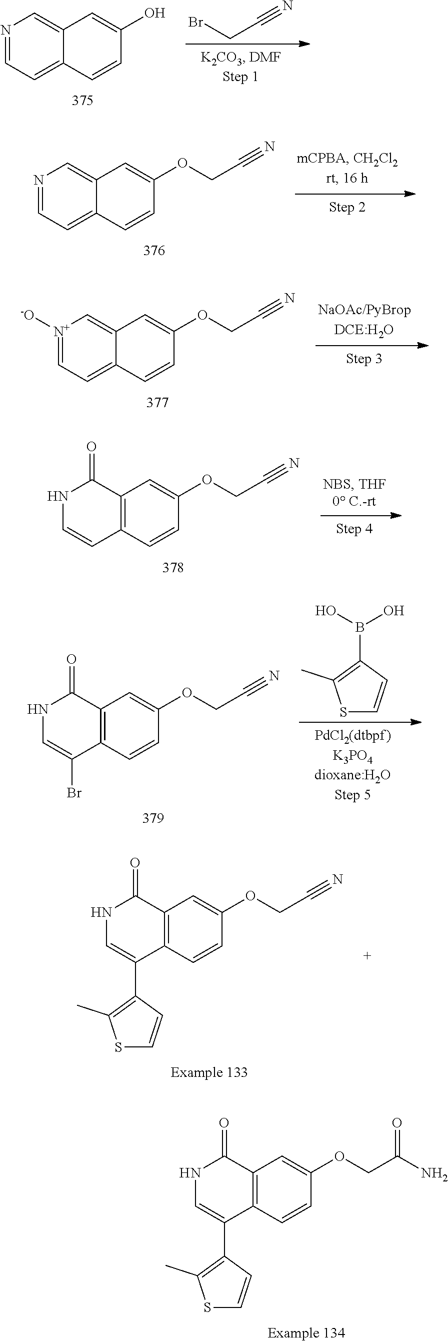

Synthesis of 2-(isoquinolin-7-yloxy)acetonitrile, 376 [Step 1]: To the stirred solution of isoquinolin-7-ol (375, 5.0 g, 34.4 nmol) and K₂CO₃ (14.3 g, 103 nmol) in DMF (30 mL) was added 2-2-bromoacetonitrile (2.6 mL, 37.9 mmol) at ambient temperature. The reaction mixture was stirred for 0.5 h. The reaction mixture was diluted with cold water and extracted with EtOAc (×3). The combined organic extracts were dried over anhydrous Na₂SO₄, filtered, and concentrated under reduced pressure to afford 2-(isoquinolin-7-yloxy)acetonitrile (376, 5.50 g). LCMS (ESI) Calcd. for C₁₁H₈N₂O: 184, found [M+H]⁺=185. ¹H NMR (400 MHz, DMSO-d₆) δ 9.25 (s, 1H), 8.45-8.43 (m, 1H), 8.0-7.95 (m, 1H), 7.82-7.80 (m, 1H), 7.71 (br s, 1H), 7.55-7.52 (m, 1H), 5.34 (s, 2H).

Synthesis of 7-(cyanomethoxy)isoquinoline 2-oxide, 377 [Step 2]: To a stirred solution of 2-(isoquinolin-7-yloxy)acetonitrile (376, 5.40 g, 29.3 mmol) in CH₂Cl₂ (80 mL) was added portion wise m-CPBA (7.2 g, 29.3 mmol) at 0° C. The reaction mixture was allowed to warm to ambient temperature and stirred for 16 h. The reaction mixture was quenched with saturated aq. NaHCO₃ and extracted with 10% MeOH in CH₂C₂. The combined organic extracts were washed with brine, dried over anhydrous Na₂SO₄, and concentrated under reduced pressure to afford 7-(cyanomethoxy)isoquinoline 2-oxide (377, 4.20 g). LCMS (ESI) Calcd. for C₁₁H₈N₂O₂: 200, found [M+H]⁺=201.

Synthesis of 2-((1-oxo-1,2-dihydroisoquinolin-7-yl)oxy)acetonitrile, 378 [Step 3]: To a suspension of 7-(cyanomethoxy)isoquinoline 2-oxide (377, 5.0 g, 25.0 mmol) in dichloroethane (120 mL) and water (30 mL) was added NaOAc (4.1 g, 50.0 mmol) and PyBroP (23.3 g, 50.0 mmol) at ambient temperature. The reaction mixture was stirred at 90° C. for 16 h. The reaction mixture was partitioned between CH₂C₂ and water. The aqueous layer was further extracted with CH₂C₁₂ (×2). The combined organic extracts were washed with brine, dried over anhydrous Na₂SO₄, filtered, and concentrated under reduced pressure. The product was purified by column chromatography to afford 2-((1-oxo-1,2-dihydroisoquinolin-7-yl)oxy)acetonitrile (378, 1.60 g). LCMS (ESI) Calcd. for C₁₁H₈N₂O₂: 200, found [M+H]⁺=201. ¹H NMR (400 MHz, DMSO-d₆) δ 11.30 (br s, 1H), 7.76-7.75 (s, 1H), 7.69-7.67 (d, 1H), 7.43-7.41 (d, 1H), 7.10 (t, 1H), 6.56-6.54 (d, 1H), 5.30-5.25 (s, 2H).

Synthesis of 2-((4-bromo-1-oxo-1,2-dihydroisoquinolin-7-yl)oxy)acetonitrile, 379 [Step 4]: To a suspension of 2-((1-oxo-1,2-dihydroisoquinolin-7-yl)oxy)acetonitrile (378, 1 g, 5.0 mmol) in THF (30 mL) was added portion wise NBS (980 mg, 5.5 nmol) at 0° C. The reaction mixture was stirred at ambient temperature for 16 h. The reaction mixture was quenched with ice cold water, filtered through a sintered funnel, and washed with water several times. The product was dried under reduced pressure to afford 2-((4-bromo-1-oxo-1,2-dihydroisoquinolin-7-yl)oxy)acetonitrile (379, 800 mg). LCMS (ESI) Calcd. for C₁₁H₇BrN₂O₂: 279, found [M+H]⁺=279. ¹H NMR (400 MHz, DMSO-d₆) δ 11.62 (br s, 1H), 7.82 (br s, 1H), 7.79-7.77 (m, 1H), 7.59-7.56 (m, 1H), 7.48 (s, 1H), 5.35 (s, 2H).

Synthesis of 2-((4-(2-methylthiophen-3-yl)-1-oxo-1,2-dihydroisoquinolin-7-yl)oxy)acetonitrile, Example 133, and 2-((4-(2-methylthiophen-3-yl)-1-oxo-1,2-dihydroisoquinolin-7-yl)oxy)acetamide, Example 134 [Step 5]: To a degassed solution of 2-((4-bromo-1-oxo-1,2-dihydroisoquinolin-7-yl)oxy)acetonitrile (379, 120 mg, 0.4 mmol) and (2-methylthiophen-3-yl)boronic acid (90 mg, 0.6 mmol) in 1,4-dioxane (5 mL) and water (1 mL) was added K₃PO₄ (230 mg, 1 mmol) at ambient temperature. The reaction mixture was degassed with nitrogen for 10 min., and PdCl₂(dtbpf) (30 mg, 0.05 mmol) was added. The reaction mixture was stirred at 100° C. for 16 h. The reaction mixture was filtered through celite and washed with EtOAc (×2). The combined organic extracts were concentrated under reduced pressure. The product was purified by reverse phase prep-HPLC and lyophilized to afford 2-((4-(2-methylthiophen-3-yl)-1-oxo-1,2-dihydroisoquinolin-7-yl)oxy)acetonitrile (Example 133, 50 mg) and 2-((4-(2-methylthiophen-3-yl)-1-oxo-1,2-dihydroisoquinolin-7-yl)oxy)acetamide (Example 134, 15 mg).

Example 133: 2-((4-(2-methylthiophen-3-yl)-1-oxo-1,2-dihydroisoquinolin-7-yl)oxy)acetonitrile: LCMS (ESI) Calcd. for C₁₆H₁₂N₂O₂S: 296, found [M−H]⁻=295. ¹H NMR (400 MHz, DMSO-d₆) δ11.49 (br s, 1H), 7.86-7.85 (m, 1H), 7.43-7.40 (m, 2H), 7.24-7.22 (m, 1H), 7.00-6.96 (m, 2H), 5.32 (s, 2H), 2.25 (s, 3H).

Example 134: 2-((4-(2-methylthiophen-3-yl)-1-oxo-1,2-dihydroisoquinolin-7-yl)oxy)acetamide: LCMS (ESI) Calcd. for C₁₆H₁₄N₂O₃S: 314, found [M+H]⁺=315. ¹H NMR (400 MHz, DMSO-d₆) δ 11.49 (br s, 1H), 7.67-7.66 (m, 1H), 7.62 (br s, 1H), 7.42-7.35 (m, 3H), 7.20-7.17 (m, 1H), 6.97 (br s, 1H), 6.93 (br s, 1H), 4.55 (s, 2H), 2.55 (br s, 3H).

### Example 135-136: Synthesis of 2-((4-(3-fluoro-2-methylphenyl)-1-oxo-1,2-dihydroisoquinolin-7-yl)oxy)acetonitrile and 2-((4-(3-fluoro-2-methylphenyl)-1-oxo-1,2-dihydroisoquinolin-7-yl)oxy)acetamide

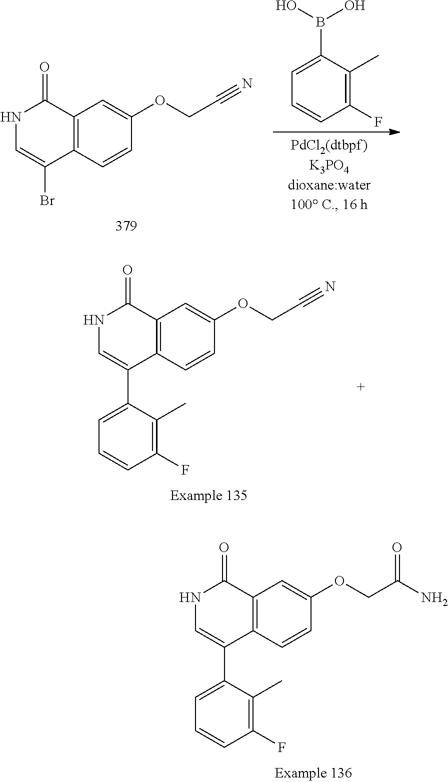

Synthesis of 2-((4-(3-fluoro-2-methylphenyl)-1-oxo-1,2-dihydroisoquinolin-7-yl)oxy)acetonitrile, Example 135, and 2-((4-(3-fluoro-2-methylphenyl)-1-oxo-1,2-dihydroisoquinolin-7-yl)oxy)acetamide, Example 136: To a degassed solution of 2-((4-bromo-1-oxo-1,2-dihydroisoquinolin-7-yl)oxy)acetonitrile (379, 120 mg, 0.4 mmol) and (3-fluoro-2-methylphenyl)boronic acid (100 mg, 0.6 mmol) in 1,4-dioxane (4 mL) and water (1 mL) was added K₃PO₄ (230 mg, 1.1 mmol) at ambient temperature. The reaction mixture was degassed with argon for 10 min., and PdCl₂(dtbpf) (30 mg, 0.04 mmol) was added. The reaction mixture was stirred at 100° C. for 16 h. The reaction mixture was filtered through celite and washed with EtOAc (×2). The combined organic extracts were concentrated under reduced pressure. The product was purified by reverse phase prep-HPLC and lyophilized to afford 2-((4-(3-fluoro-2-methylphenyl)-1-oxo-1,2-dihydroisoquinolin-7-yl)oxy)acetonitrile (Example 135, 50 mg) and 2-((4-(3-fluoro-2-methylphenyl)-1-oxo-1,2-dihydroisoquinolin-7-yl)oxy)acetamide (Example 136, 39 mg).

Example 135: 2-((4-(3-fluoro-2-methylphenyl)-1-oxo-1,2-dihydroisoquinolin-7-yl)oxy)acetonitrile: LCMS (ESI) Calcd. for C₁₈H₁₃FN₂O₂: 308, found [M+H]⁺=309. ¹H NMR (400 MHz, DMSO-d₆) δ11.53 (d, 1H), 7.87 (d, 1H), 7.38 (dd, 1H), 7.32 (t, 1H), 7.25 (t, 1H), 7.09 (d, 1H), 7.02-7.00 (m, 2H), 5.32 (s, 2H), 1.95 (s, 3H).

Example 136: 2-((4-(3-fluoro-2-methylphenyl)-1-oxo-1,2-dihydroisoquinolin-7-yl)oxy)acetamide: LCMS (ESI) Calcd. for C₁₈H₁₅FN₂O₃: 326, found [M+H]⁺=327. ¹H NMR (400 MHz, DMSO-d₆) δ 11.42 (d, 1H), 7.68 (d, 1H), 7.62 (br s, 1H), 7.39 (br s, 1H), 7.34-7.30 (m, 2H), 7.24 (t, 1H), 7.07 (d, 1H), 6.97-6.93 (m, 2H), 4.55 (s, 2H), 1.95 (s, 3H).

### Example 137-138: Synthesis of 2-((4-(2-ethylphenyl)-1-oxo-1,2-dihydroisoquinolin-7-yl)oxy)acetonitrile and 2-((4-(2-ethylphenyl)-1-oxo-1,2-dihydroisoquinolin-7-yl)oxy)acetamide

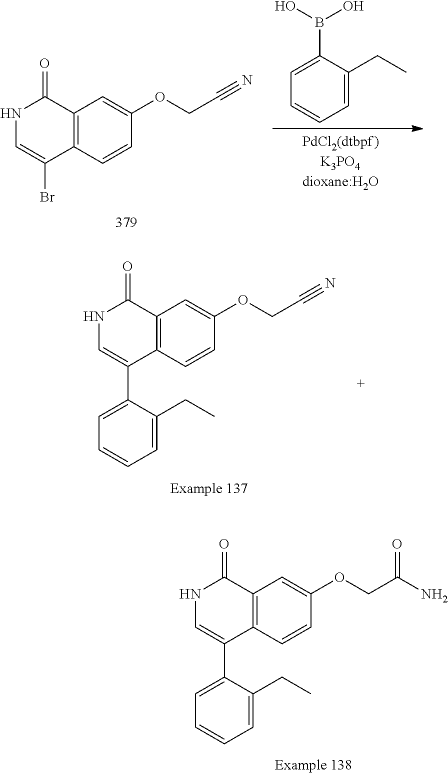

Synthesis of 2-((4-(2-ethylphenyl)-1-oxo-1,2-dihydroisoquinolin-7-yl)oxy)acetonitrile, Example 137, and 2-((4-(2-ethylphenyl)-1-oxo-1,2-dihydroisoquinolin-7-yl)oxy)acetamide, Example 138: To a degassed solution of 2-((4-bromo-1-oxo-1,2-dihydroisoquinolin-7-yl)oxy)acetonitrile (379, 120 mg, 0.4 mmol) and (2-ethylphenyl)boronic acid (97 mg, 0.6 mmol) in 1,4-dioxane (5 mL) and water (1 mL) was added K₃PO₄ (230 mg, 1 nmol) at ambient temperature. The reaction mixture was degassed with nitrogen for 10 min., and PdCl₂(dtbpf) (30 mg, 0.05 mmol) was added. The reaction mixture was stirred at 100° C. for 16 h. The reaction mixture was filtered through celite and washed with EtOAc (×2). The combined organic extracts were concentrated under reduced pressure. The product was purified by reverse phase prep-HPLC and lyophilized to afford 2-((4-(2-ethylphenyl)-1-oxo-1,2-dihydroisoquinolin-7-yl)oxy)acetonitrile (Example 137, 60 mg) and 2-((4-(2-ethylphenyl)-1-oxo-1,2-dihydroisoquinolin-7-yl)oxy)acetamide (Example 138, 15 mg).

Example 137: 2-((4-(2-ethylphenyl)-1-oxo-1,2-dihydroisoquinolin-7-yl)oxy)acetonitrile: LCMS (ESI) Calcd. for C₁₉H₁₆N₂O₂: 304, found [M+H]⁺=305. ¹H NMR (400 MHz, DMSO-d₆) δ11.47-11.45 (br s, 1H), 7.86-7.85 (d, 1H), 7.40-7.35 (m, 3H), 7.31-7.27 (m, 1H), 7.18-7.17 (d, 1H), 6.97-6.95 (m, 2H), 5.32 (s, 2H), 2.45-2.38 (m, 1H), 2.36-2.28 (m, 1H), 0.99-0.96 (t, 3H).

Example 138: 2-((4-(2-ethylphenyl)-1-oxo-1,2-dihydroisoquinolin-7-yl)oxy)acetamide: LCMS (ESI) Calcd. for C₁₉H₁₈N₂O₃: 322, found [M+H]⁺=323. ¹H NMR (400 MHz, DMSO-d₆) δ 11.36-11.34 (br s, 1H), 7.67-7.66 (m, 1H), 7.61 (br s, 1H), 7.39-7.38 (m, 3H), 7.33-7.26 (m, 21H), 7.17-7.15 (d, 11H), 6.92-6.89 (m, 2H), 4.50 (s, 2H), 2.43-2.36 (m, 1H), 2.35-2.28 (m, 1H), 0.99-0.96 (t, 3H).

### Example 139: Synthesis of (R)-4-(2-chloro-4-fluorophenyl)-7-((1-(1-imino-1-oxido-1λ⁶-thiomorpholino)-1-oxopropan-2-yl)oxy)isoquinolin-1(2H)-one

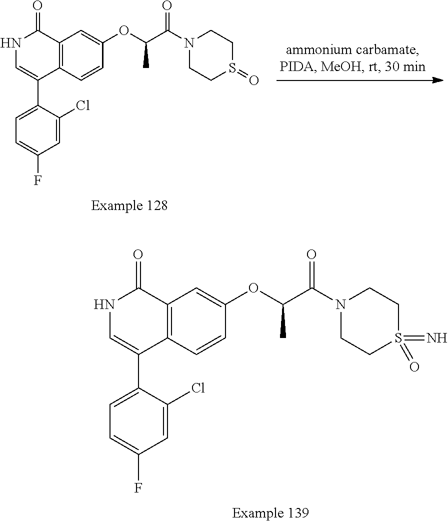 [SMILES: C[C@@H](Oc1ccc2c(-c3ccc(F)cc3Cl)c[nH]c(=O)c2c1)C(=O)N1CCS(=O)CC1 ; C[C@@H](Oc1ccc2c(-c3ccc(F)cc3Cl)c[nH]c(=O)c2c1)C(=O)N1CCS(=N)(=O)CC1]

Synthesis of (R)-4-(2-chloro-4-fluorophenyl)-7-((1-(1-imino-1-oxido-1λ⁶-thiomorpholino)-1-oxopropan-2-yl)oxy)isoquinolin-1(2H)-one, Example 139: To a stirred solution of (R)-4-(2-chloro-4-fluorophenyl)-7-((1-(1-oxidothiomorpholino)-1-oxopropan-2-yl)oxy)isoquinolin-1(2H)-one (Example 128, 135 mg, 0.3 mmol) in MeOH (3 mL) was added ammonium carbamate (90 mg, 1.2 mmol) and iodobenzene diacetate (280 mg, 0.9 mmol). The reaction mixture was stirred for 30 min. at ambient temperature in an open flask. The reaction mixture was concentrated under reduced pressure. The product was purified by reverse phase prep-HPLC and lyophilized to afford (R)-4-(2-chloro-4-fluorophenyl)-7-((1-(1-imino-1-oxido-1λ⁶-thiomorpholino)-1-oxopropan-2-yl)oxy)isoquinolin-1(2H)-one (Example 139, 37 mg). LCMS (ESI) Calcd. for C₂₂H₂₁ClFN₃O₄S: 477, found [M+H]⁺=478. ¹H NMR (400 MHz, DMSO-d₆) δ 11.49-11.47 (m, 1H), 7.62-7.59 (m, 2H), 7.50-7.45 (m, 1H), 7.36-7.32 (m, 1H), 7.28-7.26 (m, 1H), 7.01 (br s, 1H), 6.97-6.94 (m, 1H), 5.56-5.54 (m, 1H), 4.40-4.38 (m, 1H), 4.07-3.74 (m, 3H), 3.42-3.19 (m, 5H), 1.48-1.47 (m, 3H).

### Example 140: Synthesis of 2-((4-(1-methyl-TH-pyrazol-5-yl)-1-oxo-1,2-dihydroisoquinolin-7-yl)oxy)acetamide

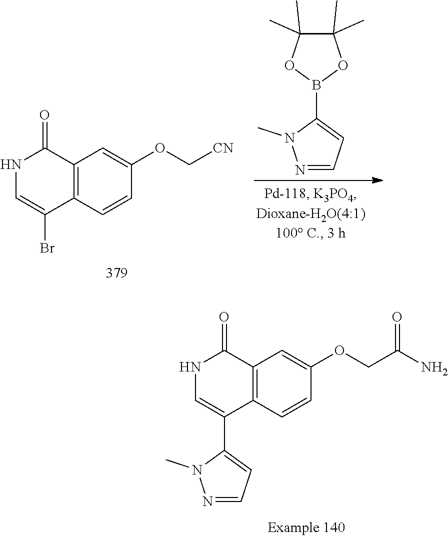

Synthesis of 2-((4-(1-methyl-1H-pyrazol-5-yl)-1-oxo-1,2-dihydroisoquinolin-7-yl)oxy)acetamide, Example 140: In a sealed tube, a stirred solution of 2-((4-bromo-1-oxo-1,2-dihydroisoquinolin-7-yl)oxy)acetonitrile (379, 120 mg, 0.4 mmol) and 1-methyl-5-(4,4,5,5-tetramethyl-1,3,2-dioxaborolan-2-yl)pyrazole (134 mg, 0.6 mmol) and K₃PO₄ (230 mg, 1.01 mmol) were dissolved in 1,4-dioxane (4 mL) and water (1 mL). The reaction mixture was purged with argon for 10 min. and PdCl₂(dtbpf) (30 mg, 0.04 mmol) was added and heated at 100° C. for 16 h. The reaction mixture was filtered through a celite bed and the filtrate was concentrated under reduced pressure. The product was purified by reverse phase HPLC to afford 2-((4-(1-methyl-1H-pyrazol-5-yl)-1-oxo-1,2-dihydroisoquinolin-7-yl)oxy)acetamide (Example 140, 44 mg). LCMS (ESI) Calcd. for C₁₅H₁₄N₄O₃: 298, found [M+H]⁺=299. ¹H NMR (400 MHz, DMSO-d₆) δ 11.59 (s, 1H), 7.67-7.64 (m, 2H), 7.55 (d, 1H), 7.40-7.37 (m, 2H), 7.16 (s, 1H), 7.11 (d, 1H), 6.35 (d, 1H), 4.56 (s, 2H), 3.66 (s, 3H).

### Example 141: Synthesis of 2-((4-(5-fluoro-2-methylphenyl)-1-oxo-1,2-dihydroisoquinolin-7-yl)oxy)acetonitrile

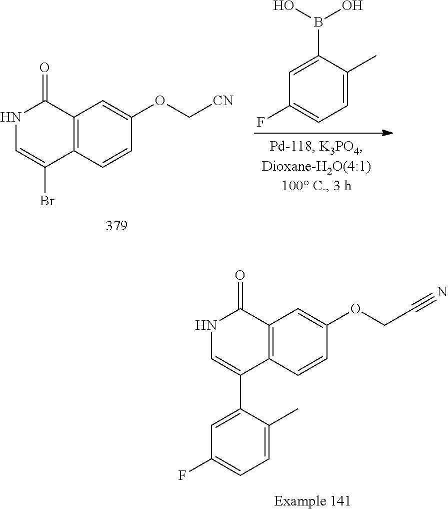 [SMILES: N#CCOc1ccc2c(Br)c[nH]c(=O)c2c1 ; Cc1ccc(F)cc1B(O)O ; Cc1ccc(F)cc1-c1c[nH]c(=O)c2cc(OCC#N)ccc12]

Synthesis of 2-((4-(5-fluoro-2-methylphenyl)-1-oxo-1,2-dihydroisoquinolin-7-yl)oxy)acetonitrile, Example 141: In a sealed tube was added 2-((4-bromo-1-oxo-1,2-dihydroisoquinolin-7-yl)oxy)acetonitrile (379, 100 mg, 0.4 mmol), (5-fluoro-2-methyl-phenyl)boronic acid (70 mg, 0.5 mmol) in 1,4-dioxane (3 mL) and water (1 mL), and K₃PO₄ (227 mg, 1.0 mmol). The reaction mixture was purged with argon for 10 min. and PdCl₂(dtbpf) (23 mg, 0.04 mmol) was added and heated at 100° C. for 3 h. The reaction mixture was filtered through a celite bed and the filtrate was concentrated under reduced pressure. The product was purified by reverse phase prep-HPLC to afford 2-((4-(5-fluoro-2-methylphenyl)-1-oxo-1,2-dihydroisoquinolin-7-yl)oxy)acetonitrile (Example 141, 60 mg). LCMS (ESI) Calcd. for C₁₈H₁₃FN₂O₂: 308, found [M+H]⁺=309. ¹H NMR (400 MHz, DMSO-d₆) δ_{H} 11.53 (s, 1H), 7.86 (d, 1H), 7.40-7.37 (m, 2H), 7.22-7.17 (m, 1H), 7.10-7.07 (m, 1H), 7.01-6.98 (m, 2H), 5.32 (s, 2H), 2.00 (s, 3H).

### Example 142: Synthesis of 2-((4-(2,6-dimethylpyridin-3-yl)-1-oxo-1,2-dihydroisoquinolin-7-yl)oxy)acetonitrile

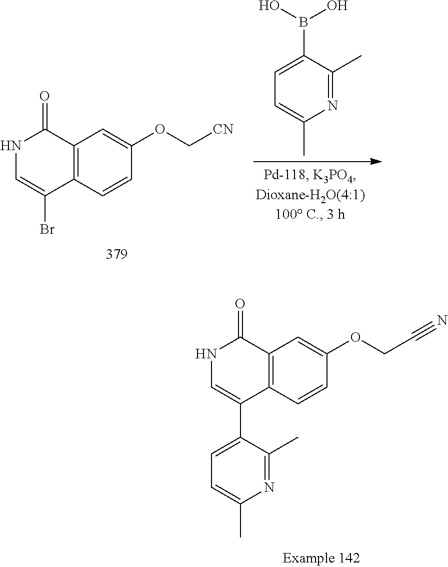 [SMILES: N#CCOc1ccc2c(Br)c[nH]c(=O)c2c1 ; Cc1ccc(B(O)O)c(C)n1 ; Cc1ccc(-c2c[nH]c(=O)c3cc(OCC#N)ccc23)c(C)n1]

Synthesis of 2-((4-(2,6-dimethylpyridin-3-yl)-1-oxo-1,2-dihydroisoquinolin-7-yl)oxy)acetonitrile, Example 142: In a sealed tube was added 2-((4-bromo-1-oxo-1,2-dihydroisoquinolin-7-yl)oxy)acetonitrile (379, 120 mg, 0.4 mmol), (2,6-dimethyl-3-pyridyl)boronic acid (95 mg, 0.6 mmol) in 1,4-dioxane (4 mL) and water (1 mL), and K₃PO₄ (228 mg, 1.0 mmol). The reaction mixture was purged with argon for 10 min. and PdCl₂(dtbpf) (30 mg, 0.04 mmol) was added and heated at 100° C. for 3 h.

The reaction mixture was filtered through a celite bed and filtrate was concentrated under reduced pressure. The product was purified by reverse phase prep-HPLC to afford 2-((4-(2,6-dimethylpyridin-3-yl)-1-oxo-1,2-dihydroisoquinolin-7-yl)oxy)acetonitrile (Example 142, 24 mg). LCMS (ESI) Calcd. for C₁₈H₁₅N₃O₂: 305, found [M+H]⁺=306. ¹H NMR (400 MHz, DMSO-d₆) δ 11.54 (s, 1H), 7.86 (d, 1H), 7.50 (d, 1H), 7.39-7.36 (m, 1H), 7.18 (d, 1H), 7.02-6.98 (m, 2H), 5.35 (s, 2H), 2.50 (s, 3H), 2.19 (s, 3H).

### Example 143: Synthesis of 2-((4-(5-fluoro-2-methylphenyl)-1-oxo-1,2-dihydroisoquinolin-7-yl)oxy)acetonitrile

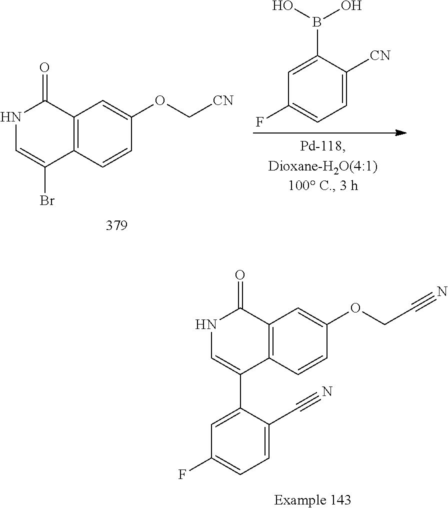 [SMILES: N#CCOc1ccc2c(Br)c[nH]c(=O)c2c1 ; N#Cc1ccc(F)cc1B(O)O ; N#CCOc1ccc2c(-c3cc(F)ccc3C#N)c[nH]c(=O)c2c1]

Synthesis of 2-((4-(5-fluoro-2-methylphenyl)-1-oxo-1,2-dihydroisoquinolin-7-yl)oxy)acetonitrile, Example 143: To a stirred solution of 2-((4-bromo-1-oxo-1,2-dihydroisoquinolin-7-yl)oxy)acetonitrile (379, 100 mg, 0.4 mmol) and (5-fluoro-2-methyl-phenyl)boronic acid (2, 70 mg, 0.5 mmol) in 1,4-dioxane (3 mL) and water (1 mL), was added K₃PO₄ (227 mg, 1.0 mmol). The reaction mixture was purged with argon for 10 min. and PdCl₂(dtbpf) (25 mg, 0.04 mmol) was added and heated at 100° C. for 3 h. The reaction mixture was filtered through a celite bed and the filtrate was concentrated under reduced pressure. The product was purified by reverse phase HPLC to afford 2-((4-(5-fluoro-2-methylphenyl)-1-oxo-1,2-dihydroisoquinolin-7-yl)oxy)acetonitrile (Example 143, 60 mg). LCMS (ESI) Calcd. for C₁₈H₁₀FN₃O₂: 319, found [M+H]⁺=320. ¹H NMR (400 MHz, DMSO-d₆) δ 11.70 (s, 1H), 8.11-8.08 (d, 1H), 7.89 (d, 1H), 7.57-7.52 (m, 2H), 7.45-7.42 (m, 1H), 7.29 (s, 1H), 7.23 (d, 1H), 5.35 (s, 2H).

### Example 144: Synthesis of 2-((4-(1-methyl-1H-pyrazol-5-yl)-1-oxo-1,2-dihydroisoquinolin-7-yl)oxy)acetonitrile

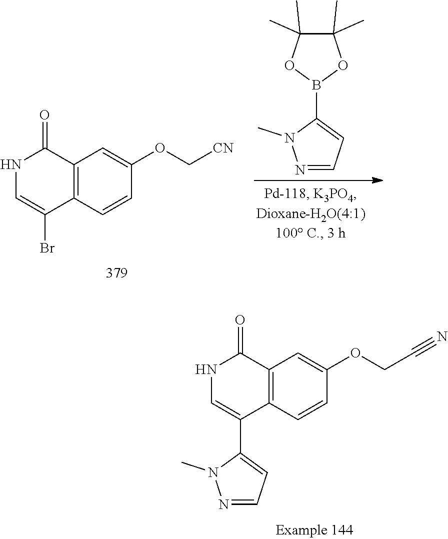

Synthesis of 2-((4-(1-methyl-1H-pyrazol-5-yl)-1-oxo-1,2-dihydroisoquinolin-7-yl)oxy)acetonitrile, Example 144: In a sealed tube was added a solution of 2-((4-bromo-1-oxo-1,2-dihydroisoquinolin-7-yl)oxy)acetonitrile (379, 80 mg, 0.3 mmol), 1-methyl-5-(4,4,5,5-tetramethyl-1,3,2-dioxaborolan-2-yl)pyrazole (2, 90 mg, 0.4 mmol) in 1,4-dioxane (4 mL) and water (1 mL), and K₃PO₄ (180 mg, 0.9 mmol). The reaction mixture was purged with argon for 10 min. and PdCl₂(dtbpf) (20 mg, 0.03 mmol) was added and heated at 100° C. for 3 h. The reaction mixture was filtered through a celite bed and the filtrate was concentrated under reduced pressure. The product was purified by reverse phase prep-HPLC to afford 2-((4-(1-methyl-1H-pyrazol-5-yl)-1-oxo-1,2-dihydroisoquinolin-7-yl)oxy)acetonitrile (Example 144, 43 mg). LCMS (ESI) Calcd. for C₁₅H₁₂N₄O₂: 280, found [M+H]⁺=281. ¹H NMR (400 MHz, DMSO-d₆) δ 11.68 (s, 1H), 7.86 (d, 1H), 7.56 (d, 1H), 7.45-7.43 (m, 1H), 7.22 (s, 1H), 7.16 (m, 1H), 6.37 (s, 1H), 5.33 (s, 2H), 3.62 (s, 3

### Example 145: Synthesis of (R)-4-(2-chloro-4-fluorophenyl)-7-((1-(1-(methylimino)-1-oxido-1λ⁶-thiomorpholino)-1-oxopropan-2-yl)oxy)isoquinolin-1(2H)-one

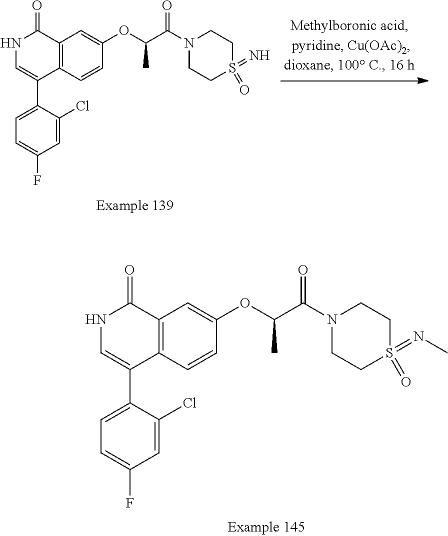 [SMILES: C[C@@H](Oc1ccc2c(-c3ccc(F)cc3Cl)c[nH]c(=O)c2c1)C(=O)N1CCS(=N)(=O)CC1 ; CN=S1(=O)CCN(C(=O)[C@@H](C)Oc2ccc3c(-c4ccc(F)cc4Cl)c[nH]c(=O)c3c2)CC1]

Synthesis of (R)-4-(2-chloro-4-fluorophenyl)-7-((1-(1-(methylimino)-1-oxido-1λ⁶-thiomorpholino)-1-oxopropan-2-yl)oxy)isoquinolin-1(2H)-one, Example 145: In a pressure tube was added (R)-4-(2-chloro-4-fluorophenyl)-7-((1-(1-imino-1-oxido-1λ⁶-thiomorpholino)-1-oxopropan-2-yl)oxy)isoquinolin-1(2H)-one (Example 139, 80 mg, 0.2 mmol), copper(II)acetate (45 mg, 0.3 mmol), pyridine (0.035 mL, 0.4 mmol) and 1,4-dioxane (2 mL), and the reaction mixture was purged with oxygen for 10 min. To the reaction mixture was added methyl boronic acid (20 mg, 0.3 mmol) and the pressure tube was closed with a teflon cap and heated at 100° C. for 16 h. The reaction mixture was concentrated under reduce pressure and the residue was diluted with ethyl acetate, washed with water, brine, dried over anhydrous Na₂SO₄, and concentrated under reduce pressure. The product was purified by reverse phase prep-HPLC and lyophilized to afford (R)-4-(2-chloro-4-fluorophenyl)-7-((1-(1-(methylimino)-1-oxido-1λ⁶-thiomorpholino)-1-oxopropan-2-yl)oxy)isoquinolin-1(2H)-one (Example 145, 56 mg). LCMS (ESI) Calcd. for C₂₃H₂₃ClFN₃O₄S: 491, found [M+H]⁺=492. ¹H NMR (400 MHz, DMSO-d₆) δ 11.49-11.47 (m, 1H), 7.62-7.60 (m, 2H), 7.50-7.45 (m, 1H), 7.36-7.32 (m, 1H), 7.28-7.26 (m, 1H), 7.01-7.00 (m, 1H), 5.56-5.53 (m, 1H), 4.31-3.68 (m, 5H), 3.20-2.97 (m, 3H), 2.66 (s, 3H), 1.47 (d, 3H).

### Example 146-147: Synthesis of chiral analogs of 1-((1-oxo-4-(o-tolyl)-1,2-dihydroisoquinolin-7-yl)-D-alanyl)piperidine-3-carboxylic acid

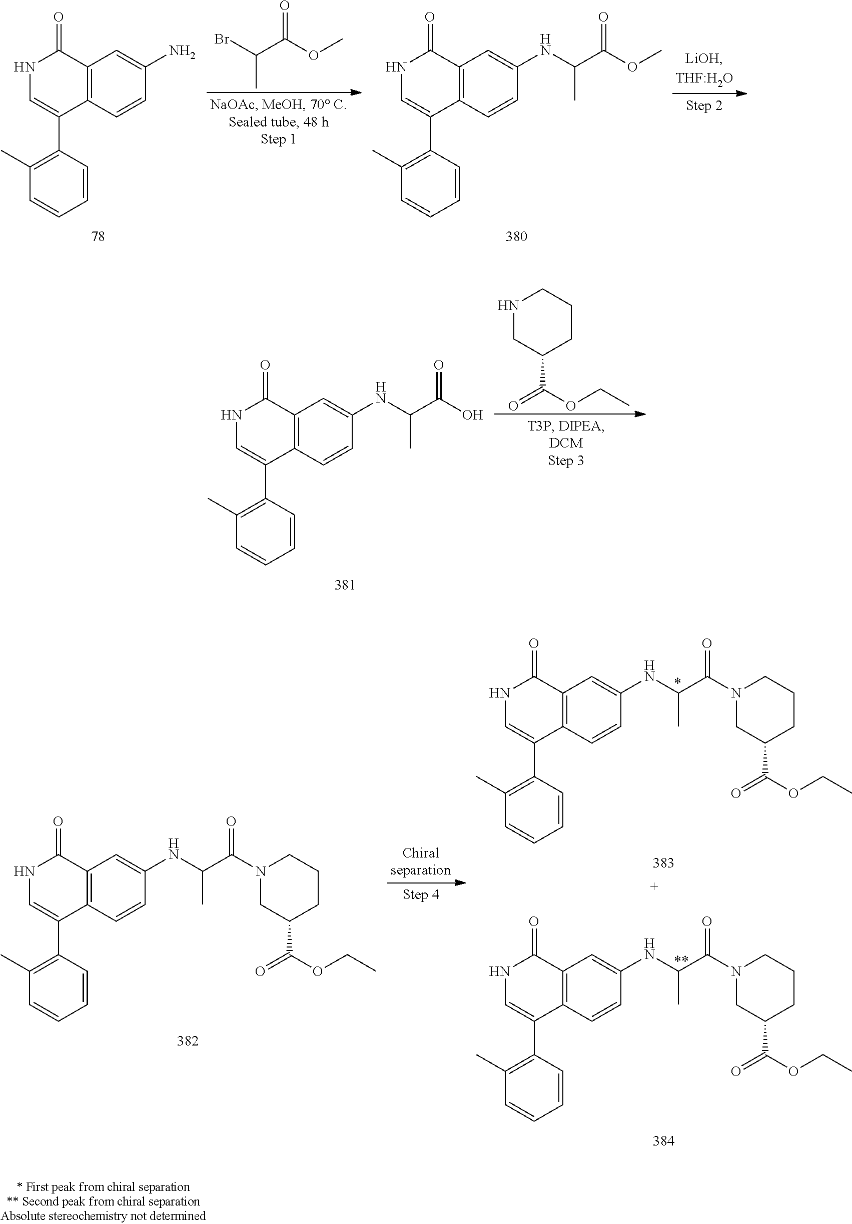

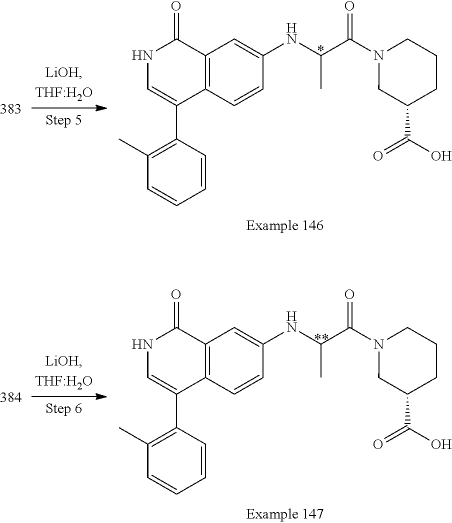 [SMILES: Cc1ccccc1-c1c[nH]c(=O)c2cc(NC(C)C(=O)N3CCC[C@H](C(=O)O)C3)ccc12 ; Cc1ccccc1-c1c[nH]c(=O)c2cc(NC(C)C(=O)N3CCC[C@H](C(=O)O)C3)ccc12]

Synthesis of methyl (1-oxo-4-(o-tolyl)-1,2-dihydroisoquinolin-7-yl)alaninate, 380 [Step 1]: In a sealed tube, to a solution of 7-amino-4-(o-tolyl)isoquinolin-1(2H)-one (78, 100 mg, 0.4 mmol) in methanol (5 mL) was added NaOAc (330 mg, 4 mmol) and methyl 2-bromopropanoate (400 mg, 2.4 mmol) and the reaction mixture was heated at 70° C. for 48 h. The reaction mixture was concentrated under reduced pressure and the mixture was quenched with water and extracted with ethyl acetate (thrice). The combined organic extracts were washed with water, brine, dried over anhydrous sodium sulphate, filtered, and concentrated under reduced pressure. The product was purified by flash column chromatography to afford methyl (1-oxo-4-(o-tolyl)-1,2-dihydroisoquinolin-7-yl)alaninate (380, 45 mg). LCMS (ESI) Calcd. for C₂₀H₂₀N₂O₃: 336, found [M+H]⁺=337.

Synthesis of (1-oxo-4-(o-tolyl)-1,2-dihydroisoquinolin-7-yl)alanine, 381 [Step 2]: To a stirred solution of methyl (1-oxo-4-(o-tolyl)-1,2-dihydroisoquinolin-7-yl)alaninate (380, 400 mg, 1.2 mmol) in THF (4 mL) was added LiOH H₂O (150 mg, 3.6 mmol) in H₂O (1.5 mL) dropwise at 0° C., and the reaction mixture was stirred for 2 h. at ambient temperature. THF was removed under reduced pressure and the residue was dissolved in water and the pH was adjusted to 5-6 by the addition of 4N HCl. The solution was lyophilized to afford (1-oxo-4-(o-tolyl)-1,2-dihydroisoquinolin-7-yl)alanine (380, 350 mg). LCMS (ESI) Calcd. for C₁₉H₁₈N₂O₃: 322, found [M+H]⁺=323.

Synthesis of ethyl (3S)-1-((1-oxo-4-(o-tolyl)-1,2-dihydroisoquinolin-7-yl)alanyl)piperidine-3-carboxylate, 382 [Step 3]: To a stirred solution of (1-oxo-4-(o-tolyl)-1,2-dihydroisoquinolin-7-yl)alanine (380, 300 mg, 0.9 mmol) in dichloromethane (5 mL), was added ethyl (S)-piperidine-3-carboxylate (220 mg, 1.4 mmol) and DIPEA (0.8 mL, 4.7 mmol) at 0° C. To this cold reaction mixture was added T₃P (0.4 mL, 1.4 mmol, 50% in EtOAc) and the reaction mixture was gradually warmed to ambient temperature and stirred for 2 h. The reaction mixture was diluted with dichloromethane and washed with water and brine, dried over anhydrous Na₂SO₄, and concentrated under reduced pressure to afford ethyl (3S)-1-((1-oxo-4-(o-tolyl)-1,2-dihydroisoquinolin-7-yl)alanyl)piperidine-3-carboxylate (382, 400 mg). LCMS (ESI) Calcd. for C₂₇H₃₁N₃O₄: 461, found [M+H]⁺=462.

Synthesis of chiral ethyl (3S)-1-((1-oxo-4-(o-tolyl)-1,2-dihydroisoquinolin-7-yl)alanyl)piperidine-3-carboxylate, 383 and 384 [Step 4]: The racemic compound ethyl (3S)-1-((1-oxo-4-(o-tolyl)-1,2-dihydroisoquinolin-7-yl)alanyl)piperidine-3-carboxylate (382, 400 mg) was purified by normal phase chiral-HPLC and lyophilized to afford the first product as chiral ethyl 1-((1-oxo-4-(o-tolyl)-1,2-dihydroisoquinolin-7-yl)alanyl)piperidine-3-carboxylate (383, 120 mg) as Peak 1 and the second product as chiral ethyl 1-((1-oxo-4-(o-tolyl)-1,2-dihydroisoquinolin-7-yl)alanyl)piperidine-3-carboxylate (384, 100 mg) as Peak 2. The absolute stereochemistry of these Examples was not determined.

Peak 1: Chiral ethyl 1-((1-oxo-4-(o-tolyl)-1,2-dihydroisoquinolin-7-yl)alanyl)piperidine-3-carboxylate, 383: LCMS (ESI) Calcd. for C₂₇H₃₁N₃O₄: 461, found [M+H]⁺=462.

Peak 2: Chiral ethyl 1-((1-oxo-4-(o-tolyl)-1,2-dihydroisoquinolin-7-yl)alanyl)piperidine-3-carboxylate, 384: LCMS (ESI) Calcd. for C₂₇H₃₁N₃O₄: 461, found [M+H]⁺=462.

Synthesis of (S)-1-((1-oxo-4-(o-tolyl)-1,2-dihydroisoquinolin-7-yl)-D-alanyl)piperidine-3-carboxylic acid, Example 146 [Step 5]: To a stirred solution of ethyl 1-((1-oxo-4-(o-tolyl)-1,2-dihydroisoquinolin-7-yl)-D-alanyl)piperidine-3-carboxylate (383, 120 mg, 0.3 mmol) in THF (5 mL), was dropwise added LiOH H₂O (45 mg, 1 mmol) in H₂O (1.5 mL) at 0° C. and the reaction mixture was stirred for 2 h. at ambient temperature. THF was removed under reduced pressure and the residue was acidified to pH 5-6 by adding 4N HCl and concentrated under reduced pressure. The product was purified by reverse phase prep-HPLC and lyophilized to afford 1-((1-oxo-4-(o-tolyl)-1,2-dihydroisoquinolin-7-yl)-D-alanyl)piperidine-3-carboxylic acid (Example 146, 45 mg). LCMS (ESI) Calcd. for C₂₅H₂₇N₃O₄: 433, found [M−H]⁻=432. ¹H NMR (400 MHz, DMSO-d₆) (at 100° C.) δ 10.50 (br s, 1H), 7.39 (br s, 1H), 7.32-7.15 (m, 4H), 7.04-7.02 (m, 1H), 6.76 (d, 1H), 6.65 (s, 1H), 5.83 (br s, 1H), 4.59 (br s, 1H), 4.18 (m, 1H), 3.83 (br s, 1H), 3.21-3.16 (m, 2H), 2.50-2.42 (m, 1H), 2.07 (s, 3H), 1.96-1.91 (m, 1H), 1.71-1.58 (m, 3H), 1.34-1.32 (m, 3H).

Synthesis of (S)-1-((1-oxo-4-(o-tolyl)-1,2-dihydroisoquinolin-7-yl)-L-alanyl)piperidine-3-carboxylic acid, Example 147 [Step 6]: To a stirred solution of ethyl 1-((1-oxo-4-(o-tolyl)-1,2-dihydroisoquinolin-7-yl)-L-alanyl)piperidine-3-carboxylate (384, 100 mg, 0.2 mmol) in THF (5 mL) was dropwise added LiOH H₂O (40 mg, 0.9 mmol) in H₂O (1.5 mL) at 0° C. and the reaction mixture was stirred for 2 h. at ambient temperature. THF was removed under reduced pressure and the residue was adjusted to pH 5-6 by the addition of 4N HCl, and the mixture was concentrated under reduced pressure. The product was purified by reverse phase prep-HPLC and the eluent was lyophilized to afford 1-((1-oxo-4-(o-tolyl)-1,2-dihydroisoquinolin-7-yl)-L-alanyl)piperidine-3-carboxylic acid (Example 147, 35 mg). LCMS (ESI) Calcd. for C₂₅H₂₇N₃O₄: 433, found [M−H]⁻=432. ¹H NMR (400 MHz, DMSO-d₆) δ 10.66 (br s, 1H), 7.38-7.23 (m, 4H), 7.17-7.15 (m, 1H), 7.03-7.00 (m, 1H), 6.75 (d, 1H), 6.65 (s, 1H), 5.85-5.83 (m, 1H), 4.58-4.42 (m, 2H), 4.02-3.98 (m, 1H), 3.00-2.64 (m, 4H), 2.07 (s, 3H), 2.03-1.98 (m, 1H), 1.72-1.61 (m, 2H), 1.45-1.42 (m, 1H), 1.34-1.32 (m, 3H).

### Example 148-149: Synthesis of chiral analogs of (S)-1-(2-methyl-3-(1-oxo-4-(o-tolyl)-1,2-dihydroisoquinolin-7-yl)propanoyl)piperidine-3-carboxylic acid

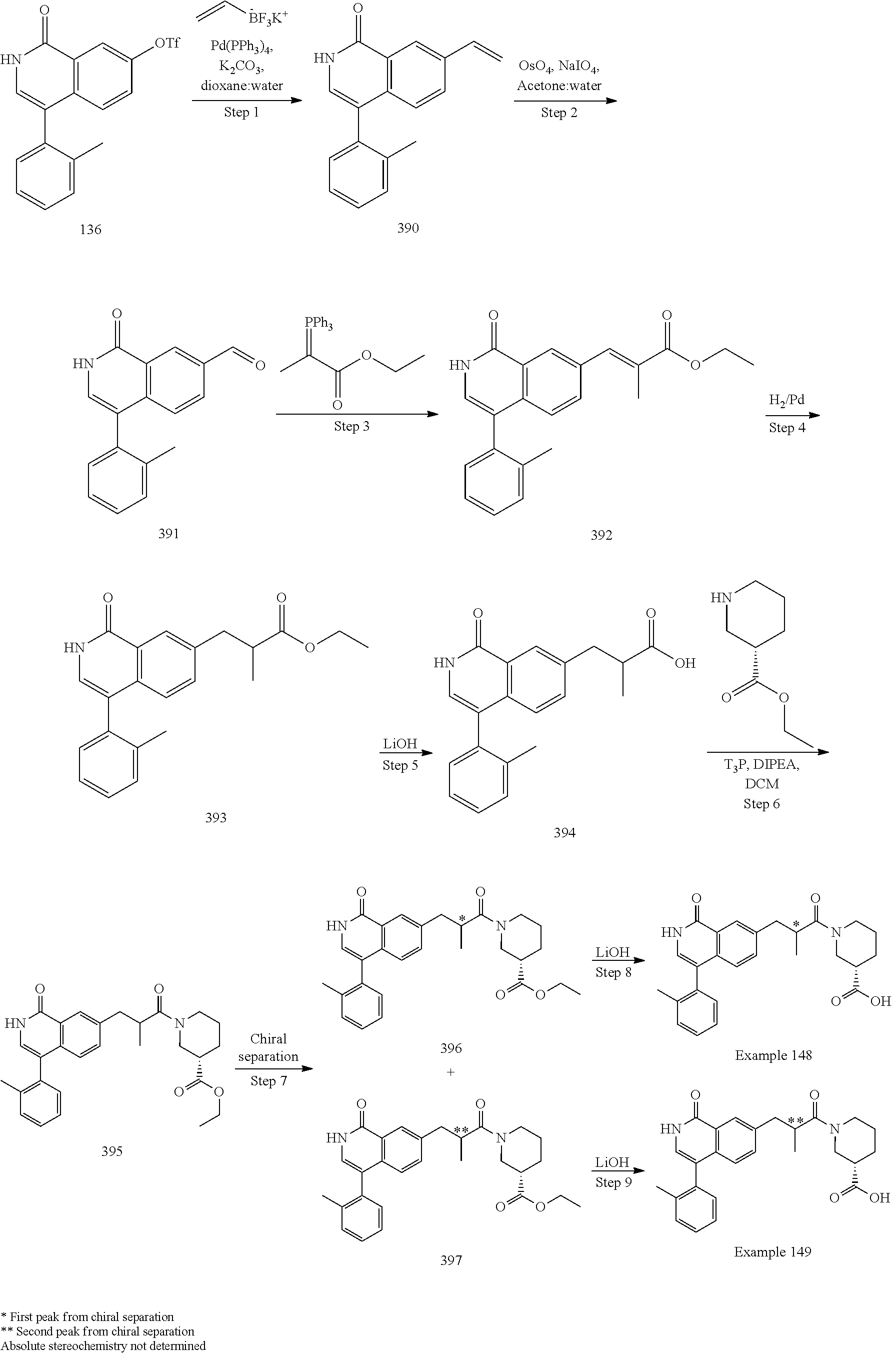

Synthesis of 4-(o-tolyl)-7-vinylisoquinolin-1(2H)-one, 390 [Step 1]: A stirred solution of 1-oxo-4-(o-tolyl)-1,2-dihydroisoquinolin-7-yl trifluoromethanesulfonate (136, 1.1 g, 2.9 mmol) in a mixture of 1,4-dioxane:water (20 mL:5 mL) was degassed with argon for 5 min. To the mixture was added potassium vinyltrifluoroborate (385 mg, 2.7 mmol) and K₂CO₃ (1.1 g, 7.9 mmol) and degassing was continued for 10 min. To the mixture was added Pd(PPh₃)₄ (330 mg, 0.3 mmol) and the reaction mixture was stirred at 110° C. for 12 h. The solution was diluted with water and extracted with ethyl acetate (twice). The organic layer was washed with brine, dried over anhydrous Na₂SO₄, and purified by flash column chromatography to afford 4-(o-tolyl)-7-vinylisoquinolin-1(2H)-one (390, 650 mg). LCMS (ESI) Calcd. for C₁₈H₁₅NO: 261, found [M+H]⁺=262. ¹H NMR (400 MHz, DMSO-d₆) δ 11.41 (d, 1H), 8.27 (s, 1H), 7.81 (d, 1H), 7.36 (d, 2H), 7.31-7.27 (m, 1H), 7.22 (d, 1H), 7.00 (t, 1H), 6.95 (t, 1H), 6.89 (t, 1H), 5.93 (d, 1H), 5.36 (d, 1H), 2.04 (s, 3H).

Synthesis of 1-oxo-4-(o-tolyl)-1,2-dihydroisoquinoline-7-carbaldehyde, 391 [Step 2]: To a solution of 4-(o-tolyl)-7-vinylisoquinolin-1(2H)-one (390, 450 mg, 1.7 mmol) in acetone (18 mL) and water (2 mL) was added N-methylmorpholine N-oxide (405 mg, 3.4 mmol) followed by the addition of OsO₄ (0.2 mL, 0.2 mmol, 4% in water). The reaction mixture was stirred at ambient temperature for 3 h. and NaIO₄ (1.6 g, 7.62 mmol) was added at 0° C. The reaction mixture was diluted with ethyl acetate and washed with water and brine. The organic layer was dried over anhydrous Na₂SO₄, and concentrated under reduced pressure. The product was purified by column chromatography to afford 1-oxo-4-(o-tolyl)-1,2-dihydroisoquinoline-7-carbaldehyde (391, 390 mg). LCMS (ESI) Calcd. for C₁₇H₁₃NO₂: 263, found [M+H]⁺=264. ¹H NMR (400 MHz, DMSO-d₆) δ 11.73 (s, 1H), 10.14 (d, 1H), 8.88 (d, 1H), 8.12 (t, 1H), 7.39-7.32 (m, 3H), 7.26 (d, 1H), 7.16-7.06 (m, 1H), 5.42 (t, 1H), 2.07 (t, 3H).

Synthesis ethyl (E)-2-methyl-3-(1-oxo-4-(o-tolyl)-1,2-dihydroisoquinolin-7-yl)acrylate, 392 [Step 3]: To a stirred solution of 1-oxo-4-(o-tolyl)-1,2-dihydroisoquinoline-7-carbaldehyde (391, 200 mg, 0.8 mmol) in toluene (10 mL) was added ethyl 2-(triphenyl-λ⁵-phosphanylidene)propanoate (0.54 g, 1.5 mmol) at ambient temperature under argon. The reaction mixture was stirred at ambient temperature for 5 h, and concentrated under reduced pressure. The product was diluted with ethyl acetate and washed with water, brine, dried over anhydrous Na₂SO₄, and concentrated under reduced pressure. The product was purified by column chromatography to afford ethyl (E)-2-methyl-3-(1-oxo-4-(o-tolyl)-1,2-dihydroisoquinolin-7-yl)acrylate (392, 200 mg). LCMS (ESI) Calcd. for C₂₂H₂₁NO₃: 347, found [M+H]⁺=348. ¹H NMR (400 MHz, DMSO-d₆) δ 11.50 (s, 1H), 8.36 (s, 1H), 8.12 (t, 1H), 7.76 (d, 1H), 7.70 (s, 2H), 7.37 (d, 1H), 7.30 (d, 1H), 7.23 (d, 1H), 7.08 (s, 1H), 7.02 (d, 1H), 4.24-4.19 (m, 1H), 2.11 (d, 6H), 1.30 (t, 3H).

Synthesis of ethyl 2-methyl-3-(1-oxo-4-(o-tolyl)-1,2-dihydroisoquinolin-7-yl)propanoate, 393 [Step 4]: A stirred solution of ethyl (E)-2-methyl-3-(1-oxo-4-(o-tolyl)-1,2-dihydroisoquinolin-7-yl)acrylate (392, 420 mg, 1.2 mmol) in ethanol (30 mL) was degassed with argon for 10 min. To the solution was added Pd—C (120 mg, 1.2 mmol, 10%) under argon and the mixture was hydrogenated using a hydrogen gas balloon for 3 h. The reaction mixture was filtered through a celite bed, washed with ethanol, and the filtrate was concentrated under reduced pressure. The product was purified by column chromatography to afford ethyl 2-methyl-3-(1-oxo-4-(o-tolyl)-1,2-dihydroisoquinolin-7-yl)propanoate (393, 350 mg). LCMS (ESI) Calcd. for C₂₂H₂₃NO₃: 349, found [M+H]⁺=350. ¹H NMR (400 MHz, DMSO-d₆) δ 11.33 (d, 1H), 8.07 (s, 1H), 7.46 (d, 1H), 7.35 (s, 2H), 7.27 (s, 1H), 7.20 (t, 1H), 6.95 (s, 1H), 6.90 (d, 1H), 4.00-3.96 (m, 2H), 2.96 (d, 1H), 2.86-2.75 (m, 2H), 2.03 (s, 3H), 1.12-1.04 (m, 3H).

Synthesis of 2-methyl-3-(1-oxo-4-(o-tolyl)-1,2-dihydroisoquinolin-7-yl)propanoic acid, 394 [Step 5]: A stirred solution of ethyl 2-methyl-3-(1-oxo-4-(o-tolyl)-1,2-dihydroisoquinolin-7-yl)propanoate (393, 280 mg, 0.8 mmol) in THF (5 mL) was cooled to 0° C. LiOH H₂O (100 mg, 2.4 mmol) in water (1 mL) was added to the reaction mixture and stirring was continued at ambient temperature for 5 h. The mixture was concentrated under reduced pressure and acidified using 1N HCl to pH 3-4 at 0° C. The product was filtered, washed with water, n-pentane, and dried under reduced pressure to afford 2-methyl-3-(1-oxo-4-(o-tolyl)-1,2-dihydroisoquinolin-7-yl)propanoic acid (394, 230 mg). LCMS (ESI) Calcd. for C₂₀H₁₉NO₃: 321, found [M+H]⁺=322. ¹H NMR (400 MHz, DMSO-d₆) 12.19 (s, 1H), 11.34 (d, 1H), 8.09 (s, 1H), 7.49 (d, 1H), 7.35 (s, 2H), 7.29 (t, 1H), 7.20 (d, 1H), 6.96 (d, 1H), 6.90 (d, 1H), 3.16 (s, 2H), 3.01 (t, 1H), 2.76 (d, 1H), 2.67 (d, 1H), 2.04 (s, 3H).

Synthesis of ethyl (3S)-1-(2-methyl-3-(1-oxo-4-(o-tolyl)-1,2-dihydroisoquinolin-7-yl)propanoyl)piperidine-3-carboxylate, 395 [Step 6]: A stirred solution of 2-methyl-3-(1-oxo-4-(o-tolyl)-1,2-dihydroisoquinolin-7-yl)propanoic acid (394, 180 mg, 0.6 mmol) and methyl (3S)-piperidine-3-carboxylate (95 mg, 0.7 mmol) in dichloromethane (10 mL) was cooled to 0° C. To the mixture was added dropwise DIPEA (0.3 mL, 1.7 mmol) and T₃P (0.4 mL, 0.8 mmol, 50% in ethyl acetate) and the temperature was raised to ambient temperature and stirring was continued for 4 h. The reaction mixture was quenched with ice water and extracted with dichloromethane, washed with brine, dried over anhydrous Na₂SO₄, and concentrated under reduced pressure. The product was purified by column chromatography to afford ethyl (3S)-1-(2-methyl-3-(1-oxo-4-(o-tolyl)-1,2-dihydroisoquinolin-7-yl)propanoyl)piperidine-3-carboxylate (395, 240 mg). LCMS (ESI) Calcd. for C₂₇H₃₀N₂O₄: 360, found [M+H]⁺=361. ¹H NMR (400 MHz, DMSO-d₆) δ 11.30 (s, 1H), 8.09 (d, 1H), 7.47 (d, 1H), 7.35 (d, 2H), 7.28 (s, 1H), 7.18 (d, 1H), 6.94 (d, 1H), 6.87 (d, 1H), 5.75 (s, 1H), 4.35 (s, 1H), 4.05 (t, 3H), 3.77 (t, 1H), 3.19 (s, 1H), 2.94 (s, 1H), 2.87 (s, 2H), 2.75 (d, 2H), 2.03 (s, 3H), 1.53 (d, 2H), 1.23-1.13 (m, 3H), 1.11-1.02 (m, 3H).

Synthesis of chiral ethyl (3S)-1-(2-methyl-3-(1-oxo-4-(o-tolyl)-1,2-dihydroisoquinolin-7-yl)propanoyl)piperidine-3-carboxylate, 396 and 397 [Step 7]: Ethyl (3S)-1-(2-methyl-3-(1-oxo-4-(o-tolyl)-1,2-dihydroisoquinolin-7-yl)propanoyl)piperidine-3-carboxylate (395, 250 mg, 0.5 mmol) was separated using SFC chiral chromatography to afford Peak 1 as chiral ethyl (3S)-1-(2-methyl-3-(1-oxo-4-(o-tolyl)-1,2-dihydroisoquinolin-7-yl)propanoyl)piperidine-3-carboxylate (396, 90 mg) and Peak 2 as chiral ethyl (35)-1-(2-methyl-3-(1-oxo-4-(o-tolyl)-1,2-dihydroisoquinolin-7-yl)propanoyl)piperidine-3-carboxylate (397, 90 mg). The absolute stereochemistry of these products was not determined.

396: Chiral ethyl (35)-1-(2-methyl-3-(1-oxo-4-(o-tolyl)-1,2-dihydroisoquinolin-7-yl)propanoyl)piperidine-3-carboxylate, Peak 1: LCMS (ESI) Calcd. for C₂₇H₃₀N₂O₄: 460, found [M+H]⁺=461.

397: Chiral ethyl (35)-1-(2-methyl-3-(1-oxo-4-(o-tolyl)-1,2-dihydroisoquinolin-7-yl)propanoyl)piperidine-3-carboxylate, Peak 2: LCMS (ESI) Calcd. for C₂₇H₃₀N₂O₄:460, found [M+H]⁺=461.

Prep SFC chromatography was performed on a PIC-SOLUTION-175 instrument using a Reflect (R,R) WHELK-01 column (21.1×250 mm), 5p, operating at 35° C., at a flow rate of 60 mL/min. The mobile phase was 80% CO₂ in super critical state and 20% of 0.3% isopropylamine in MeOH, isocratic for 20 min. and isobaric at 100 bar with a 220 nm wavelength detection.

Synthesis of (S)-1-(2-methyl-3-(1-oxo-4-(o-tolyl)-1,2-dihydroisoquinolin-7-yl)propanoyl)piperidine-3-carboxylic acid, Example 148 [Step 8]: To an ice-cooled solution of chiral ethyl (3S)-1-(2-methyl-3-(1-oxo-4-(o-tolyl)-1,2-dihydroisoquinolin-7-yl)propanoyl)piperidine-3-carboxylate (396, 90 mg, 0.2 mmol) in THF (5 mL) was added LiOH H₂O (25 mg, 0.6 mmol) in water (1 mL) and the mixture was stirred at ambient temperature for 12 h. The reaction mixture was concentrated under reduced pressure and acidified with 1N HCl to pH 2-3. The product was filtered, purified by reverse phase prep-HPLC, and lyophilized to afford (S)-1-(2-methyl-3-(1-oxo-4-(o-tolyl)-1,2-dihydroisoquinolin-7-yl)propanoyl)piperidine-3-carboxylic acid (Example 148, 40 mg). LCMS (ESI) Calcd. for C₂₆H₂₈N₂O₄: 432, found [M+H]⁺=433. ¹H NMR (400 MHz, DMSO-d₆) δ 11.32 (s, 1H), 8.10 (d, 1H), 7.43 (s, 1H), 7.33 (s, 2H), 7.27 (s, 1H), 7.20 (d, 1H), 6.91 (d, 1H), 6.88 (d, 1H), 3.86 (s, 2H) 3.21 (t, 2H), 2.97 (t, 3H), 2.86 (d, 1H), 2.06 (s, 3H), 1.91 (s, 1H), 1.78 (s, 1H), 1.62 (s, 1H), 1.27 (s, 1H), 1.06 (d, 3H).

Synthesis of (S)-1-(2-methyl-3-(1-oxo-4-(o-tolyl)-1,2-dihydroisoquinolin-7-yl)propanoyl)piperidine-3-carboxylic acid, Example 149 [Step 9]: To an ice-cooled solution of chiral ethyl (3S)-1-(2-methyl-3-(1-oxo-4-(o-tolyl)-1,2-dihydroisoquinolin-7-yl)propanoyl)piperidine-3-carboxylate (397, 90 mg, 0.2 mmol) in THF (5 mL) was added LiOH H₂O (25 mg, 0.6 mmol) in water (1 mL) and the reaction mixture was stirred at ambient temperature for 12 h. The reaction mixture was concentrated under reduced pressure and acidified with 1N HCl to pH 2-3. The product was filtered, purified by reverse phase prep-HPLC, and lyophilized to afford (S)-1-(2-methyl-3-(1-oxo-4-(o-tolyl)-1,2-dihydroisoquinolin-7-yl)propanoyl)piperidine-3-carboxylic acid (Example 149, 40 mg). LCMS (ESI) Calcd. for C₂₆H₂₈N₂O₄: 432, found [M+H]⁺=433. ¹H NMR (400 MHz, DMSO-d₆) δ 11.31 (s, 1H), 8.09 (d, 1H), 7.50 (t, 1H), 7.35 (d, 2H), 7.28 (s, 1H), 7.20 (d, 1H), 6.94 (s, 1H), 6.88 (d, 1H), 3.72 (s, 2H) 3.28 (t, 2H), 2.97 (t, 3H), 2.88 (d, 1H), 2.02 (s, 3H), 1.91 (s, 1H), 1.75 (s, 1H), 1.62 (s, 1H), 1.43 (s, 1H), 1.05 (d, 3H).

### Example 150-151: Synthesis of 2-((4-(cyclohex-1-en-1-yl)-1-oxo-1,2-dihydroisoquinolin-7-yl)oxy)acetonitrile and 2-((4-(cyclohex-1-en-1-yl)-1-oxo-1,2-dihydroisoquinolin-7-yl)oxy)acetamide

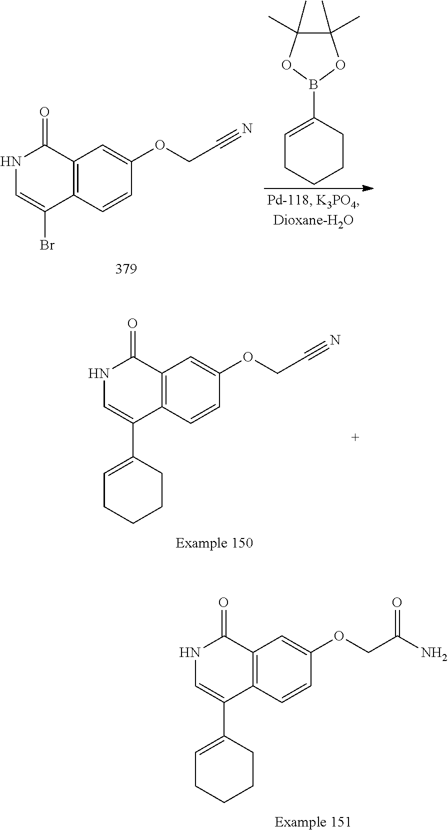

Synthesis of 2-((4-(cyclohex-1-en-1-yl)-1-oxo-1,2-dihydroisoquinolin-7-yl)oxy)acetonitrile, Example 150, and 2-((4-(cyclohex-1-en-1-yl)-1-oxo-1,2-dihydroisoquinolin-7-yl)oxy)acetamide, Example 151: To a degassed solution of 2-((4-bromo-1-oxo-1,2-dihydroisoquinolin-7-yl)oxy)acetonitrile (379, 150 mg, 0.5 mmol) and 2-(cyclohex-1-en-1-yl)-4,4,5,5-tetramethyl-1,3,2-dioxaborolane (170 mg, 0.8 mmol) in 1,4-dioxane (4 mL) and water (1 mL), was added K₃PO₄ (285 mg, 1.3 mmol) at ambient temperature. The reaction mixture was degassed with nitrogen for 10 min. and PdCl₂(dtbpf) (35 mg, 0.05 mmol) was added. The temperature was increased to 100° C. and stirred for 16 h. The reaction mixture was filtered through a celite bed and washed with ethyl acetate (twice). The combined organic extracts was concentrated under reduced pressure and the product was purified by reverse phase prep-HPLC and lyophilized to afford 2-((4-(cyclohex-1-en-1-yl)-1-oxo-1,2-dihydroisoquinolin-7-yl)oxy)acetonitrile (Example 150, 60 mg) and 2-((4-(cyclohex-1-en-1-yl)-1-oxo-1,2-dihydroisoquinolin-7-yl)oxy)acetamide (Example 151, 20 mg).

Example 150: 2-((4-(cyclohex-1-en-1-yl)-1-oxo-1,2-dihydroisoquinolin-7-yl)oxy)acetonitrile: LCMS (ESI) Calcd. for C₁₇H₁₆N₂O₂: 280, found [M−H]⁻=279. ¹H NMR (400 MHz, DMSO-d₆) δ 11.26 (br s, 1H), 7.80 (d, 1H), 7.61-7.59 (m, 1H), 7.45-7.42 (m, 1H), 6.84 (s, 1H), 5.71 (s, 1H), 5.31 (s, 2H), 2.20-2.18 (m, 4H), 1.74-1.66 (m, 4H).

Example 151: 2-((4-(cyclohex-1-en-1-yl)-1-oxo-1,2-dihydroisoquinolin-7-yl)oxy)acetamide: LCMS (ESI) Calcd. for C₁₇H₁₈N₂O₃: 298, found [M+H]⁺=299. ¹H NMR (400 MHz, DMSO-d₆) δ 11.15 (br s, 1H), 7.62-7.61 (d, 2H), 7.56-7.54 (m, 1H), 7.39-7.36 (m, 2H), 6.78-6.76 (m, 1H), 5.70 (s, 1H), 4.54 (s, 2H), 2.19-2.17 (m, 4H), 1.74-1.73 (m, 2H), 1.67-1.66 (m, 2H).

### Example 152-153: Synthesis of chiral analogs of (S)-1-((4-(2-chloro-4-fluorophenyl)-1-oxo-1,2-dihydroisoquinolin-7-yl)alanyl)piperidine-3-carboxylic acid

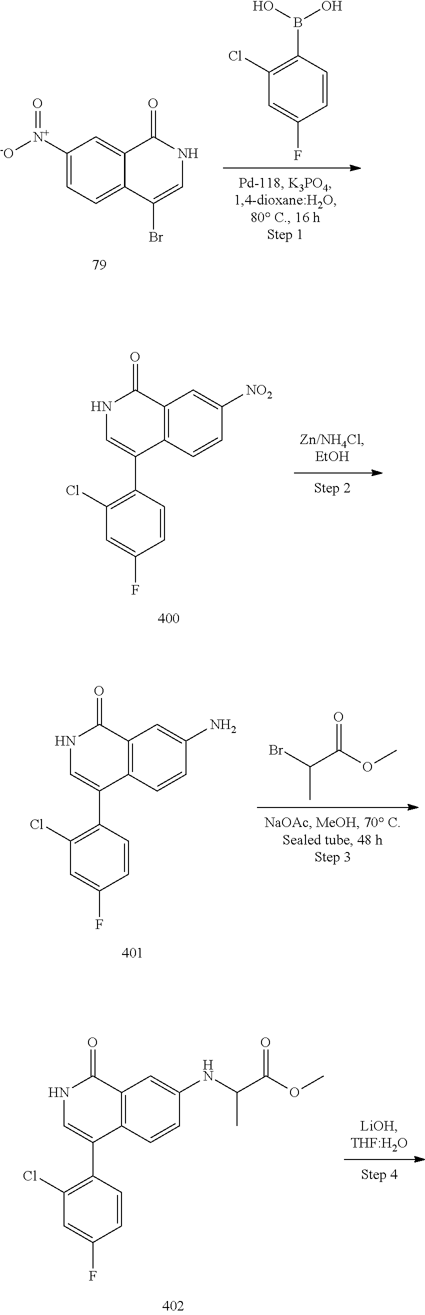

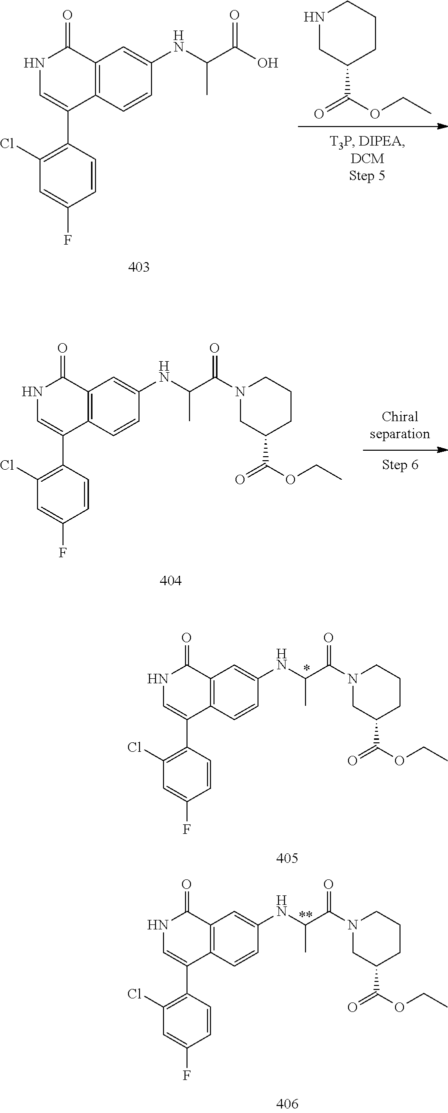 [SMILES: CC(Nc1ccc2c(-c3ccc(F)cc3Cl)c[nH]c(=O)c2c1)C(=O)O ; CCOC(=O)[C@H]1CCCNC1 ; CCOC(=O)[C@H]1CCCN(C(=O)C(C)Nc2ccc3c(-c4ccc(F)cc4Cl)c[nH]c(=O)c3c2)C1 ; CCOC(=O)[C@H]1CCCN(C(=O)C(C)Nc2ccc3c(-c4ccc(F)cc4Cl)c[nH]c(=O)c3c2)C1 ; CCOC(=O)[C@H]1CCCN(C(=O)C(C)Nc2ccc3c(-c4ccc(F)cc4Cl)c[nH]c(=O)c3c2)C1]

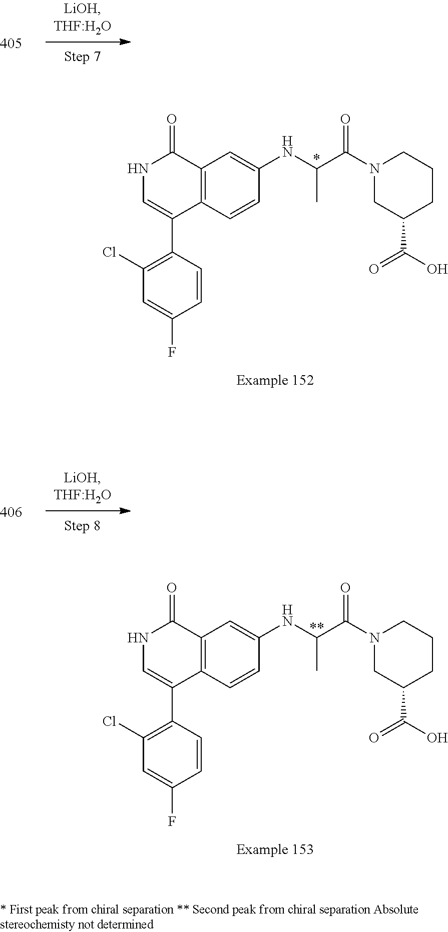 [SMILES: CC(Nc1ccc2c(-c3ccc(F)cc3Cl)c[nH]c(=O)c2c1)C(=O)N1CCC[C@H](C(=O)O)C1 ; CC(Nc1ccc2c(-c3ccc(F)cc3Cl)c[nH]c(=O)c2c1)C(=O)N1CCC[C@H](C(=O)O)C1]

Synthesis of 4-(2-chloro-4-fluorophenyl)-7-nitroisoquinolin-1(2H)-one, 400 [Step 1]: A solution of 4-bromo-7-nitroisoquinolin-1(2H)-one (79, 3.0 g, 11.0 mmol), (2-chloro-4-fluoro-phenyl)boronic acid (2.9 g, 17.0 mmol) and K₃PO₄ (4.7 g, 22 mmol) in 1,4-dioxane:water (40 mL:10 mL) was purged with nitrogen for 5 min. Pd-118 (720 mg, 1.1 mmol) was added to the reaction mixture and stirred at 100° C. for 18 h. The reaction mixture was diluted with ethyl acetate, washed with water and brine. The combined organic extracts was concentrated under reduced pressure and the product was purified by flash column chromatography to afford 4-(2-chloro-4-fluorophenyl)-7-nitroisoquinolin-1(2H)-one (400, 700 mg). LCMS (ESI) Calcd. for C₁₅H₈ClFN₂O₃: 318, found [M+H]⁺=319.

Synthesis of 7-amino-4-(2-chloro-4-fluorophenyl)isoquinolin-1(2H)-one, 401 [Step 2]: To a stirred solution of 4-(2-chloro-4-fluorophenyl)-7-nitroisoquinolin-1(2H)-one (400, 1.0 g, 3.14 mmol) in ethanol (20 mL) was added Zn dust (2.05 g, 31.4 mmol) and NH₄Cl (2.5 g, 47 mmol). The reaction mixture was stirred at ambient temperature for 18 h, and concentrated under reduced pressure. The reaction mixture was quenched with water, extracted with ethyl acetate, and washed with water and brine. The combined organic extracts were concentrated under reduced pressure to afford 7-amino-4-(2-chloro-4-fluorophenyl)isoquinolin-1(2H)-one (401, 500 mg). LCMS (ESI) Calcd. for C₁₅H₁₀ClFN₂O: 289, found [M+H]⁺=289.

Synthesis of methyl (4-(2-chloro-4-fluorophenyl)-1-oxo-1,2-dihydroisoquinolin-7-yl)alaninate, 402 [Step 3]: In a sealed tube was added 7-amino-4-(2-chloro-4-fluorophenyl)isoquinolin-1(2H)-one (401, 600 mg, 2.1 mmol), methanol (25 mL), sodium acetate (1.7 g, 20.7 mmol), and methyl 2-bromopropanoate (2.08 g, 12.47 mmol) and the reaction mixture was heated at 70° C. for 48 h. The reaction mixture was concentrated under reduced pressure and quenched with water and extracted with ethyl acetate. The organic extract was washed with water, brine, dried over anhydrous Na₂SO₄, and concentrated under reduced pressure. The product was purified by column chromatography to afford methyl (4-(2-chloro-4-fluorophenyl)-1-oxo-1,2-dihydroisoquinolin-7-yl)alaninate (402, 200 mg). LCMS (ESI) Calcd. for C₁₉H₁₆ClFN₂O₃: 375, found [M+H]⁺=375.

Synthesis of (4-(2-chloro-4-fluorophenyl)-1-oxo-1,2-dihydroisoquinolin-7-yl)alanine, 403 [Step 4]: To a stirred solution of methyl (4-(2-chloro-4-fluorophenyl)-1-oxo-1,2-dihydroisoquinolin-7-yl)alaninate (402, 320 mg, 0.8 mmol) in THF (6 mL) was dropwise added LiOH H₂O (110 mg, 2.6 mmol) in H₂O (1.5 mL) at 0° C. and stirring was continued for 2 h. at ambient temperature. The reaction mixture was concentrated reduced pressure, the pH was adjusted to 5-6 using 4N HCl, and the product was lyophilized to afford (4-(2-chloro-4-fluorophenyl)-1-oxo-1,2-dihydroisoquinolin-7-yl)alanine (403, 300 mg). LCMS (ESI) Calcd. for C₁₈H₁₄ClFN₂O₃: 361, found [M+H]⁺=361.

Synthesis of ethyl (3S)-1-((4-(2-chloro-4-fluorophenyl)-1-oxo-1,2-dihydroisoquinolin-7-yl)alanyl)piperidine-3-carboxylate, 404 [Step 5]: To a stirred solution of (4-(2-chloro-4-fluorophenyl)-1-oxo-1,2-dihydroisoquinolin-7-yl)alanine (403, 300 mg, 0.9 mmol) in dichloromethane (7 mL), was added ethyl (3S)-piperidine-3-carboxylate (195 mg, 1.2 mmol) and DIPEA (0.7 mL, 4.2 mmol) at 0° C. To this cold reaction mixture was added T₃P (0.4 mL, 1.25 mmol, 50% in ethyl acetate) and the reaction mixture was gradually warmed to ambient temperature and stirred for 2 h. The reaction mixture was diluted with dichloromethane and washed with water, brine, dried over anhydrous Na₂SO₄, and concentrated under reduced pressure to afford ethyl (3S)-1-((4-(2-chloro-4-fluorophenyl)-1-oxo-1,2-dihydroisoquinolin-7-yl)alanyl)piperidine-3-carboxylate (404, 400 mg). LCMS (ESI) Calcd. for C₂₆H₂₇ClFN₃O₄: 500, found [M+H]+=500.

Synthesis of chiral ethyl (3S)-1-((4-(2-chloro-4-fluorophenyl)-1-oxo-1,2-dihydroisoquinolin-7-yl)alanyl)piperidine-3-carboxylate, 405 and 406 [Step 6]: Ethyl (3S)-1-((4-(2-chloro-4-fluorophenyl)-1-oxo-1,2-dihydroisoquinolin-7-yl)alanyl)piperidine-3-carboxylate (16, 400 mg) was purified by normal phase chiral-HPLC and lyophilized to afford Peak 1 as chiral ethyl (3S)-1-((4-(2-chloro-4-fluorophenyl)-1-oxo-1,2-dihydroisoquinolin-7-yl)alanyl)piperidine-3-carboxylate (405, 100 mg) and Peak 2 as chiral ethyl (3S)-1-((4-(2-chloro-4-fluorophenyl)-1-oxo-1,2-dihydroisoquinolin-7-yl)alanyl)piperidine-3-carboxylate (406, 90 mg). The absolute stereochemistry of these products was not determined.

405: Chiral ethyl (3S)-1-((4-(2-chloro-4-fluorophenyl)-1-oxo-1,2-dihydroisoquinolin-7-yl)alanyl)piperidine-3-carboxylate, Peak 1: LCMS (ESI) Calcd. for C₂₆H₂₇ClFN₃O₄: 500, found [M+H]⁺=500.

406: Chiral ethyl (3S)-1-((4-(2-chloro-4-fluorophenyl)-1-oxo-1,2-dihydroisoquinolin-7-yl)alanyl)piperidine-3-carboxylate, Peak 2: LCMS (ESI) Calcd. for C₂₆H₂₇ClFN₃O₄: 500, found [M+H]⁺=500.

Synthesis of (S)-1-((4-(2-chloro-4-fluorophenyl)-1-oxo-1,2-dihydroisoquinolin-7-yl)alanyl)piperidine-3-carboxylic acid, Example 152 [Step 7]: To a stirred solution of chiral ethyl (3S)-1-((4-(2-chloro-4-fluorophenyl)-1-oxo-1,2-dihydroisoquinolin-7-yl)alanyl)piperidine-3-carboxylate (405, 100 mg, 0.2 mmol) in THF (5 mL) was dropwise added LiOH H₂O (34 mg, 0.8 mmol) in H₂O (1.5 mL) at 0° C. and stirring was continued for 2 h. at ambient temperature. The reaction mixture was concentrated under reduced pressure and the pH was adjusted to 5-6 using 4N HCl, and the product was lyophilized. The product was purified by reverse phase prep-HPLC and lyophilized to afford (5)-1-((4-(2-chloro-4-fluorophenyl)-1-oxo-1,2-dihydroisoquinolin-7-yl)alanyl)piperidine-3-carboxylic acid (Example 152, 35 mg). LCMS (ESI) Calcd. for C₂₄H₂₃ClFN₃O₄: 472, found [M−H]-=470. ¹H NMR (400 MHz, DMSO-d₆) (at 100° C.) δ 7.50-7.36 (m, 3H), 7.30-7.25 (m, 1H), 7.06 (d, 1H), 6.78 (d, 1H), 6.74 (s, 1H), 5.94 (br s, 1H), 4.59 (br s, 1H), 3.87 (br s, 1H), 3.24-3.16 (m, 2H), 2.43 (br s, 3H), 1.96 (br s, 1H), 1.68 (br s, 3H), 1.33-1.31 (m, 3H).

Synthesis of (S)-1-((4-(2-chloro-4-fluorophenyl)-1-oxo-1,2-dihydroisoquinolin-7-yl)alanyl)piperidine-3-carboxylic acid, Example 153 [Step 8]: To a stirred solution of chiral ethyl (3S)-1-((4-(2-chloro-4-fluorophenyl)-1-oxo-1,2-dihydroisoquinolin-7-yl)alanyl)piperidine-3-carboxylate (406, 90 mg, 0.2 mmol) in THF (5 mL) was dropwise added LiOH H₂O (30 mg, 0.72 mmol) in H₂O (1.5 mL) at 0° C. and stirring was continued for 2 h. at ambient temperature. The reaction mixture was concentrated under reduced pressure and the pH was adjusted to 5-6 using 4N HCl, and the product was lyophilized. The product was purified by reverse phase prep-HPLC and lyophilized to afford (5)-1-((4-(2-chloro-4-fluorophenyl)-1-oxo-1,2-dihydroisoquinolin-7-yl)alanyl)piperidine-3-carboxylic acid (Example 153, 35 mg). LCMS (ESI) Calcd. for C₂₄H₂₃ClFN₃O₄: 472, found [M−H]⁻=470. ¹H NMR (400 MHz, DMSO-d₆) (at 100° C.) δ 10.82 (br s, 1H), 7.48-7.38 (m, 3H), 7.28 (t, 1H), 7.04 (d, 1H), 6.78 (d, 1H), 6.74 (s, 1H), 5.88 (br s, 1H), 4.57 (br s, 1H), 4.23-4.21 (m, 1H), 4.02-3.99 (m, 1H), 2.53-2.50 (s, 4H), 2.02-1.98 (m, 1H), 1.69-1.61 (m, 2H), 1.48-1.42 (m, 1H), 1.34-1.32 (m, 3H).

The chemical synthesis of representative quinolinone compounds is shown below.

### Examples Q1-Q2: Synthesis of 4-(2-chloro-4-fluorophenyl)-7-methoxyquinolin-2(11H)-one and 4-(2-chloro-4-fluorophenyl)-7-hydroxyquinolin-2(1H)-one

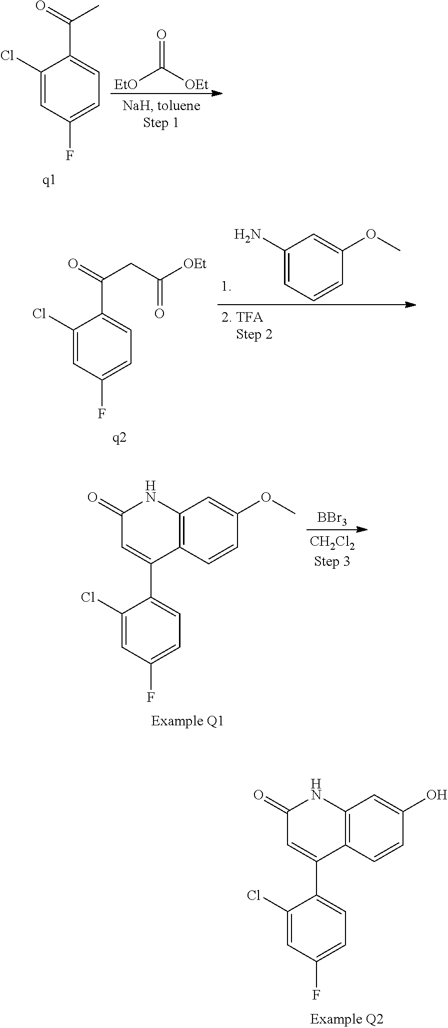

Synthesis of ethyl 3-(2-chloro-4-fluorophenyl)-3-oxopropanoate, q2 [Step 1]: To a suspension of NaH (1.11 g, 46.4 mmol, 60% dispersion in mineral oil, washed with n-pentane) in toluene (30 mL) was added dropwise diethyl carbonate (4.11 g, 34.8 mmol) at 0-10° C. and under inert atmosphere. To the reaction mixture was added dropwise 1-(2-chloro-4-fluorophenyl)ethan-1-one (q1, 2.00 g, 11.6 mmol). The reaction mixture was gradually warmed to ambient temperature and then heated to 70° C. for 4 h. The reaction mixture was cooled to 0° C., quenched with saturated aq. NH₄Cl solution, and extracted with EtOAc (×3). The combined organic layers were washed with water, brine, dried over anhydrous Na₂SO₄, filtered, and concentrated under reduced pressure. The product was purified by flash column chromatography on silica gel to give ethyl 3-(2-chloro-4-fluoro-phenyl)-3-oxo-propanoate (q2, 1.50 g). LCMS (ESI) Calcd. for C₁₁H₁₀ClFO₃: 244, found [M+H]⁺=245.

Synthesis of 4-(2-chloro-4-fluorophenyl)-7-methoxyquinolin-2(1H)-one, Example Q1 [Step 2]: A mixture of ethyl 3-(2-chloro-4-fluoro-phenyl)-3-oxo-propanoate (q2, 500 mg, 2.04 mmol) and 3-methoxy aniline (252 mg, 2.04 mmol) was heated to 140° C. for 5 h in a sealed tube. The reaction mixture was cooled to 90° C., and 1,4-dioxane (2 mL) was added. The reaction mixture was cooled to ambient temperature. The reaction mixture was concentrated under reduced pressure. The reaction mixture was partitioned between EtOAc and water. The aqueous layer was separated and further extracted with EtOAc (×2). The combined organic layers were washed with brine, dried over anhydrous Na₂SO₄, filtered, and concentrated under reduced pressure. The intermediate product was purified by flash column chromatography on silica gel (280 mg isolated). Trifluoroacetic acid (4.66 g, 40.9 mmol) was added dropwise to the intermediate product. The mixture was heated to 70° C. and stirred for 2 h. The reaction mixture was cooled to 0° C., quenched with ice cold water, basified with saturated aq. NaHCO₃ solution, and extracted with CH₂Cl₂ (×3). The combined organic layers were washed with brine, dried over anhydrous Na₂SO₄, filtered, and concentrated under reduced pressure. The final product was purified by flash column chromatography on silica gel. The product was further purified by reverse phase prep-HPLC and lyophilized to afford 4-(2-chloro-4-fluorophenyl)-7-methoxyquinolin-2(1H)-one (Example Q1, 50 mg). LCMS (ESI) Calcd. for C₁₆H₁₁ClFNO₂: 303, found [M+H]⁺=304. ¹H NMR (400 MHz, DMSO-d₆) δ 11.80 (br s, 1H), 7.65 (dd, 1H), 7.51-7.47 (m, 1H), 7.41-7.36 (dt, 1H), 6.89 (d, 1H), 6.87 (d, 1H), 6.75 (dd, 1H), 6.20 (s, 1H), 3.80 (s, 3H).

Synthesis of 4-(2-chloro-4-fluorophenyl)-7-hydroxyquinolin-2(1H)-one, Example Q2 [Step 3]: To a stirred solution of 4-(2-chloro-4-fluorophenyl)-7-methoxyquinolin-2(1H)-one (Example Q1, 350 mg, 1.15 mmol) in CH₂Cl₂ (4 mL) was added dropwise BBr₃ (1.7 g, 6.91 mmol) at 0° C. The reaction mixture was gradually warmed to ambient temperature and stirred for 24 h. The reaction mixture was cooled to 0° C. and quenched with MeOH. The reaction mixture was concentrated under reduced pressure. The product was purified by flash column chromatography on silica gel and lyophilized to afford 4-(2-chloro-4-fluorophenyl)-7-hydroxyquinolin-2(1H)-one (Example Q2, 40 mg). LCMS (ESI) Calcd. for C₁₅H₉ClFNO₂: 289, found [M+H]⁺=290. ¹H NMR (400 MHz, DMSO-d₆) δ 11.29 (s, 1H), 9.99 (s, 1H), 7.60-7.58 (m, 2H), 7.48-7.45 (q, 1H), 7.35-7.30 (m, 1H), 7.14-7.11 (m, 1H), 6.89-6.85 (m, 2H).

### Example Q3: Synthesis of 4-(4-fluoro-2-methyl-phenyl)-7-isopropoxyquinolin-2(1H)-one

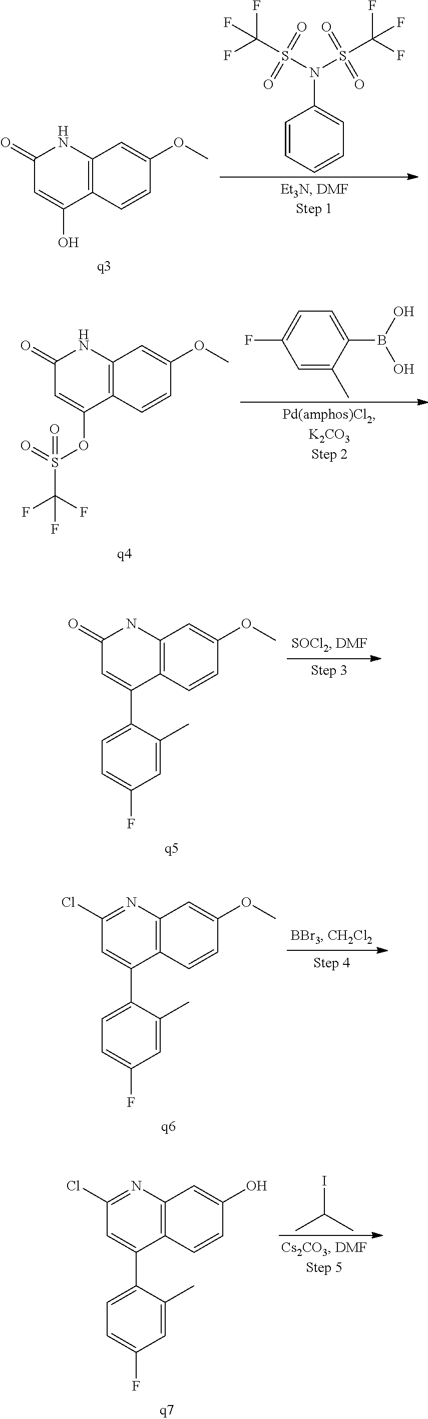

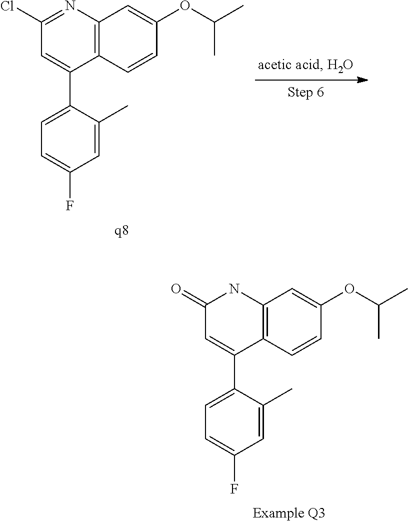 [SMILES: Cc1cc(F)ccc1-c1cc(Cl)nc2cc(OC(C)C)ccc12 ; Cc1cc(F)ccc1-c1cc(=O)[nH]c2cc(OC(C)C)ccc12]

Synthesis of 7-methoxy-2-oxo-1,2-dihydroquinolin-4-yl trifluoromethanesulfonate, q4 [Step 1]: To a suspension of 4-hydroxy-7-methoxyquinolin-2(1H)-one (q3, 1.0 g, 5.2 mmol) in DMF (20 mL) was added triethylamine (2.2 mL, 15.7 mmol) followed by 1,1,1-trifluoro-N-phenyl-N-(trifluoromethylsulfonyl)methanesulfonamide (2.2 g, 6.3 mmol). The reaction mixture was stirred at ambient temperature for 6 h. The reaction mixture was quenched with water and stirred for 30 min. The product was filtered, washed with water, and dried under reduced pressure to afford 7-methoxy-2-oxo-1,2-dihydroquinolin-4-yl trifluoromethanesulfonate (q4, 900 mg). LCMS (ESI) Calcd. for C₁₁H₈F₃NO₅S: 323, found [M+H]⁺=324. ¹H NMR (400 MHz, DMSO-d₆) δ 12.16 (s, 1H), 7.56 (d, 1H), 7.00 (dd, 1H), 6.92 (m, 1H), 6.51 (s, 1H), 3.85 (s, 3H).

Synthesis of 4-(4-fluoro-2-methylphenyl)-7-methoxyquinolin-2(1H)-one, q5 [Step 2]: To a stirred solution of (4-fluoro-2-methyl-phenyl)boronic acid (190 mg, 1.2 mmol) and 7-methoxy-2-oxo-1,2-dihydroquinolin-4-yl trifluoromethanesulfonate (q4, 400 mg, 1.24 mmol) in toluene (3.5 mL) and water (0.5 mL) was added K₂CO₃ (430 mg, 3.0 mmol) in a sealed tube. The mixture was degassed with nitrogen for 5 min., and Pd(amphos)Cl₂ (90 mg, 0.12 mmol) was added. The reaction mixture was heated to 110° C. for 12 h. The reaction mixture was cooled and partitioned between EtOAc and water. The combined organic layers were separated, washed with brine, dried over anhydrous Na₂SO₄, filtered, and concentrated under reduced pressure. The product was purified by flash chromatography on silica gel to afford 4-(4-fluoro-2-methylphenyl)-7-methoxyquinolin-2(1H)-one (q5, 242 mg). LCMS (ESI) Calcd. for C₁₇H₁₄FNO₂: 283, found [M+H]⁺=284. ¹H NMR (400 MHz, DMSO-d₆) δ 11.74 (s, 1H), 7.25-7.21 (m, 2H), 7.17-7.12 (td, 1H), 6.89-6.88 (d, 1H), 6.83-6.79 (m, 1H), 6.73-6.7 (dd, 1H), 6.13 (s, 1H), 3.80 (s, 3H), 2.05 (s, 3H).

Synthesis of 2-chloro-4-(4-fluoro-2-methylphenyl)-7-methoxyquinoline, q6 [Step 3]: To a stirred solution of 4-(4-fluoro-2-methylphenyl)-7-methoxyquinolin-2(1H)-one (q5, 240 mg, 0.8 mmol) in DMF (0.065 mL, 0.8 mmol) was added SOCl₂ (3.0 mL, 41.8 mmol). The reaction mixture was heated at 50° C. for 16 h. The reaction mixture was cooled and concentrated under reduced pressure. The reaction mixture was partitioned between EtOAc and water. The organic layer was separated, washed with water, brine, dried over anhydrous Na₂SO₄, filtered, and concentrated under reduced pressure. The product was purified by flash column chromatography on silica gel to afford 2-chloro-4-(4-fluoro-2-methylphenyl)-7-methoxyquinoline (q6, 210 mg). LCMS (ESI) Calcd. for C₁₇H₁₃ClFNO: 301, found [M+H]⁺=302.

Synthesis of 2-chloro-4-(4-fluoro-2-methylphenyl)quinolin-7-ol, q7 [Step 4]: To a stirred solution of 2-chloro-7-methoxy-4-(o-tolyl)quinoline (q6, 205 mg, 0.7 mmol) in CH₂Cl₂ (5 mL) was added BBr₃ (2.8 mL, 2.8 mmol, 1M in CH₂C₂). The reaction mixture was stirred at ambient temperature for 12 h. The reaction mixture was quenched with MeOH and concentrated under reduced pressure. The product was purified by reverse phase prep-HPLC and lyophilized to afford 2-chloro-4-(4-fluoro-2-methylphenyl)quinolin-7-ol (q7, 70 mg). LCMS (ESI) Calcd. for C₁₆H₁₁ClFNO: 287, found [M+H]⁺=288. ¹H NMR (400 MHz, DMSO-d₆) δ 10.51 (br s, 1H), 7.31-7.27 (m, 2H), 7.24-7.12 (m, 5H), 1.99 (s, 3H).

Synthesis of 2-chloro-4-(4-fluoro-2-methylphenyl)-7-isopropoxyquinoline, q8 [Step 5]: To a stirred solution of 2-chloro-4-(4-fluoro-2-methylphenyl)quinolin-7-ol (q7, 60 mg, 0.2 mmol) in DMF (3 mL) was added K₂CO₃ (72 mg, 0.5 mmol) followed by isopropyl iodide (71 mg, 0.4 mmol). The reaction mixture was heated at 70° C. for 3 h. The reaction mixture was cooled and partitioned between EtOAc and water. The organic layer was separated, washed with water (×4) and brine, dried over anhydrous Na₂SO₄, filtered, and concentrated under reduced pressure. The product was purified by flash column chromatography on silica gel to afford 2-chloro-4-(4-fluoro-2-methylphenyl)-7-isopropoxyquinoline (q8, 63 mg). LCMS (ESI) Calcd. for C₁₉H₁₇ClFNO: 329, found [M+H]⁺=330. ¹H NMR (400 MHz, DMSO-d₆) δ 7.49-7.19 (m, 7H), 4.85 (m, 1H), 1.99 (s, 3H), 1.34 (d, 6H).

Synthesis of 4-(4-fluoro-2-methyl-phenyl)-7-isopropoxyquinolin-2(1H)-one, Example Q3 [Step 6]: To a mixture of 2-chloro-4-(4-fluoro-2-methylphenyl)-7-isopropoxy-quinoline (q8, 57 mg, 0.17 mmol) in acetic acid (1.5 mL, 25.9 mmol) was added water (0.3 mL). The reaction mixture was heated to reflux for 6 h. The reaction mixture was cooled and concentrated under reduced pressure. The product was purified by reverse phase prep-HPLC and lyophilized to afford 4-(4-fluoro-2-methyl-phenyl)-7-isopropoxyquinolin-2(1H)-one (Example Q3, 32 mg). LCMS (ESI) Calcd. for C₁₉H₁₈FNO₂: 311, found [M+H]⁺=312. ¹H NMR (400 MHz, DMSO-d₆) δ 11.68 (s, 1H), 7.25-7.14 (m, 3H), 6.87 (s, 1H), 6.81 (d, 1H), 6.68 (dd, 1H), 6.11 (s, 1H), 4.6 (q, 1H), 2.05 (s, 3H), 1.30 (d, 6H).

### Example Q4: Synthesis of 4-(2-chloro-4-fluorophenyl)-7-isopropoxyquinolin-2(1H)-one

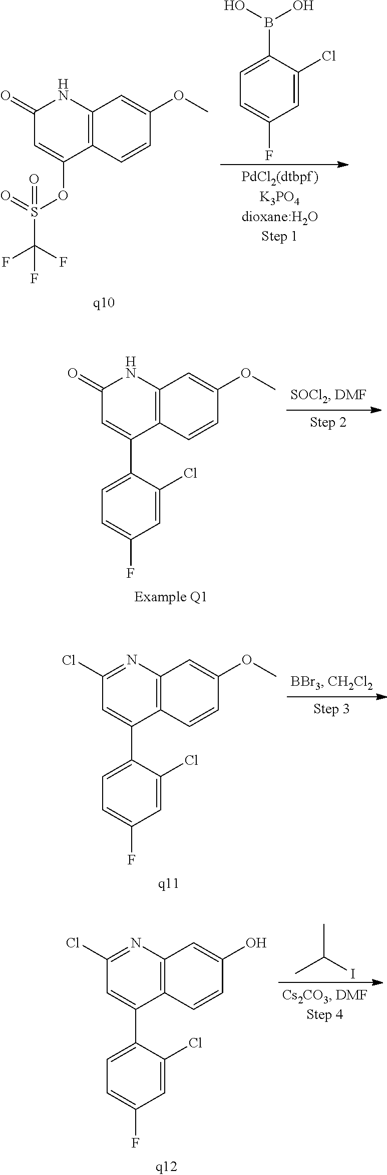

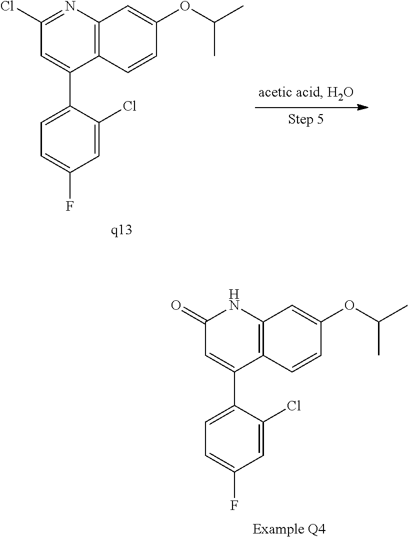 [SMILES: CC(C)Oc1ccc2c(-c3ccc(F)cc3Cl)cc(Cl)nc2c1 ; CC(C)Oc1ccc2c(-c3ccc(F)cc3Cl)cc(=O)[nH]c2c1]

Synthesis of 4-(2-chloro-4-fluorophenyl)-7-methoxyquinolin-2(1H)-one, Example Q1 [Step 1]: To a stirred solution of 7-methoxy-2-oxo-1,2-dihydroquinolin-4-yl trifluoromethanesulfonate (q10, 500 mg, 1.6 mmol) and ((2-chloro-4-fluoro-phenyl)boronic acid (405 mg, 2.3 mmol) in 1,4-dioxane (4 mL) and water (1 mL) was added K₃PO₄ (821 mg, 3.9 mmol) in a sealed tube. The reaction mixture was degassed with nitrogen for 5 min., and PdCl₂(dtbpf) (101 mg, 0.16 mmol) was added. The reaction mixture was heated at 80° C. for 12 h. The reaction mixture was cooled, dissolved in EtOAc, washed with water, brine, dried over anhydrous Na₂SO₄, filtered, and concentrated under reduced pressure. The product was triturated with methyl tert-butyl ether (MTBE) and dried to afford 4-(2-chloro-4-fluorophenyl)-7-methoxyquinolin-2(1H)-one (Example Q1, 250 mg). LCMS (ESI) Calcd. for C₁₆H₁₁ClFNO₂: 302, found [M+H]+=303. ¹H NMR (400 MHz, DMSO-d₆) δ 11.81 (s, 1H), 7.66-7.63 (dd, 1H), 7.51-7.47 (m, 1H), 7.41-7.36 (m, 1H), 6.89-6.84 (m, 2H), 6.75-6.72 (dd, 1H), 6.20 (s, 1H), 3.80 (s, 3H).

Synthesis of 2-chloro-4-(2-chloro-4-fluorophenyl)-7-methoxyquinoline, q11 [Step 2]: To a stirred solution of 4-(2-chloro-4-fluoro-phenyl)-7-methoxyquinolin-2(1H)-one (Example Q1, 150 mg, 0.5 mmol) in DMF (0.04 mL, 0.5 mmol) was added SOCl₂ (1.8 mL, 24.7 mmol). The reaction mixture was heated at 50° C. for 2 h. The reaction mixture was cooled and concentrated under reduced pressure. The reaction mixture was quenched with an aq. solution of NaHCO₃ and extracted with EtOAc (×3). The combined organic extracts were washed with water (2×50 mL) and brine, dried over anhydrous Na₂SO₄, filtered, and concentrated under reduced pressure. The product was purified by flash column chromatography on silica gel to afford 2-chloro-4-(2-chloro-4-fluorophenyl)-7-methoxyquinoline (q11, 100 mg). LCMS (ESI) Calcd. for C₁₆H₁₀Cl₂FNO: 321, found [M+H]⁺=322. ¹H NMR (400 MHz, DMSO-d₆) δ 7.72-7.71 (d, 1H), 7.59-7.56 (m, 1H), 7.46-7.40 (m, 3H), 7.32-7.25 (m, 2H), 3.93 (s, 3H).

Synthesis of 2-chloro-4-(2-chloro-4-fluorophenyl)quinolin-7-ol, q12 [Step 3]: To a solution of 2-chloro-4-(2-chloro-4-fluoro-phenyl)-7-methoxy-quinoline (q11, 150 mg, 0.47 mmol) in CH₂Cl₂ (5 mL) was added a solution of BBr₃ (583 mg, 2.4 mmol, 1M in CH₂Cl₂) at 0° C. The reaction mixture was stirred at ambient temperature for 16 h. The reaction mixture was concentrated under reduced pressure. The product was purified by flash column chromatography on silica gel to afford 2-chloro-4-(2-chloro-4-fluorophenyl)quinolin-7-ol (q12, 90 mg). LCMS (ESI) Calcd. for C₁₅H₈Cl₂FNO: 306, found [M+H]⁺=307. ¹H NMR (400 MHz, DMSO-d₆) δ 10.55 (s, 1H), 7.72-7.69 (d, 1H), 7.60-7.53 (m, 1H), 7.46-7.41 (m, 1H), 7.29-7.25 (m, 2H), 7.17-7.15 (d, 1H).

Synthesis of 2-chloro-4-(2-chloro-4-fluorophenyl)-7-isopropoxyquinoline, q13 [Step 4]: To a solution of 2-chloro-4-(2-chloro-4-fluoro-phenyl)quinolin-7-ol (q12, 95 mg, 0.31 mmol) in DMF (4 mL) was added Cs₂CO₃ (250 mg, 0.78 mmol) followed by 2-iodopropane (68 mg, 0.4 mmol). The reaction mixture was stirred at 100° C. for 2 h. The reaction mixture was partitioned between EtOAc and water. The organic layer was separated, washed with water (×3) and brine, dried over anhydrous Na₂SO₄, filtered, and concentrated under reduced pressure to afford 2-chloro-4-(2-chloro-4-fluorophenyl)-7-isopropoxyquinoline (q13, 90 mg). LCMS (ESI) Calcd. for C₁₈H₁₄Cl₂FNO: 348, found [M+H]⁺=349. ¹H NMR (400 MHz, DMSO-d₆) δ 7.72 (d, 1H), 7.57 (m, 1H), 7.44-7.38 (m, 2H), 7.30-7.27 (m, 1H), 7.25-7.22 (m, 1H), 4.88-4.86 (m, 1H), 1.24-1.23 (s, 6H).

Synthesis of 4-(2-chloro-4-fluorophenyl)-7-isopropoxyquinolin-2(1H)-one, Example Q4 [Step 5]: To a stirred solution of 2-chloro-4-(2-chloro-4-fluoro-phenyl)-7-isopropoxy-quinoline (q13, 90 mg, 0.26 mmol) in acetic acid (2.2 mL, 38.5 mmol) was added water (0.5 mL). The reaction mixture was heated to reflux at 110° C. for 16 h. The reaction mixture was cooled and concentrated under reduced pressure. The product was purified by reverse phase prep-HPLC and lyophilized to afford 4-(2-chloro-4-fluorophenyl)-7-isopropoxyquinolin-2(1H)-one (Example Q4, 25 mg). LCMS (ESI) Calcd. for C₁₈H₁₅ClFNO₂: 331, found [M+H]⁺=332. ¹H NMR (400 MHz, DMSO-d₆) δ 11.80 (s, 1H), 7.65-7.63 (d, 1H), 7.50-7.46 (m, 1H), 7.40-7.36 (m, 1H), 6.87-6.82 (m, 2H), 6.70-6.69 (d, 1H), 6.19 (s, 1H), 4.63-4.60 (m, 1H), 1.30-1.29 (s, 6H).

### Example Q5: Synthesis of (R)-4-(2-chloro-4-fluorophenyl)-7-((1-oxo-1-(piperidin-1-yl)propan-2-yl)oxy)quinolin-2(1H)-one

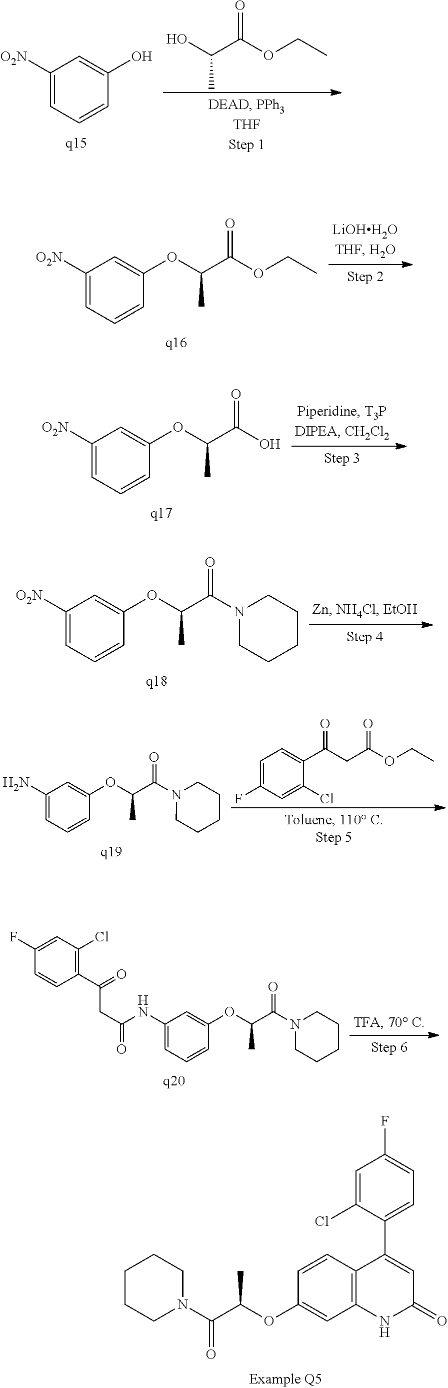

Synthesis of ethyl (R)-2-(3-nitrophenoxy)propanoate, q16 [Step 1]: To a stirred solution of 3-nitrophenol (q15, 500 mg, 3.6 mmol), triphenylphosphine (1.0 g, 4.0 mmol) and ethyl (S)-2-hydroxypropanoate (425 mg, 3.6 mmol) in dry THF (10 mL) was added 4 Å molecular sieves (500 mg) followed by DEAD (0.68 mL, 4.3 mmol) dropwise at 0° C. under an inert atmosphere. The reaction mixture was gradually heated to 60° C. and stirred for 16 h. The reaction mixture was cooled to ambient temperature and filtered. The filtrate was collected and concentrated under reduced pressure. The product was purified by flash column chromatography to afford ethyl (R)-2-(3-nitrophenoxy)propanoate (q16, 500 mg). ¹H NMR (400 MHz, DMSO-d₆) δ 7.83 (d, 1H), 7.67 (s, 1H), 7.58 (t, 1H), 7.41-7.39 (m, 1H), 5.20 (q, 1H), 4.16 (q, 2H), 1.55 (d, 3H), 1.17 (t, 3H).

Synthesis of (R)-2-(3-nitrophenoxy)propanoic acid, q17 [Step 2]: To a solution of ethyl (R)-2-(3-nitrophenoxy)propanoate (q16, 500 mg, 2.1 mmol) in THF (5 mL) was added a solution of LiOH·H₂O (263 mg, 6.3 mmol) in water (1 mL). The reaction mixture was stirred at ambient temperature for 6 h. The reaction mixture was concentrated under reduced pressure. The reaction mixture was neutralized with 2M HCl, filtered, washed with water and n-pentane. The reaction mixture was concentrated under reduced pressure to afford (R)-2-(3-nitrophenoxy)propanoic acid (q17, 300 mg). ¹H NMR (400 MHz, DMSO-d₆) δ 13.26 (br s, 1H), 7.82 (d, 1H), 7.65 (br s, 1H), 7.58 (t, 1H), 7.38 (dd, 1H), 5.07 (q, 1H), 1.53 (d, 3H).

Synthesis of (R)-2-(3-nitrophenoxy)-1-(piperidin-1-yl)propan-1-one, q18 [Step 3]: To a stirred solution of (R)-2-(3-nitrophenoxy)propanoic acid (q17, 180 mg, 0.852 mmol), piperidine (0.13 mL, 1.28 mmol) and DIPEA (0.37 mL, 2.13 mmol) in CH₂Cl₂ (3 mL) was added dropwise T₃P (1 mL, 0.938 mmol, 50% in EtOAc) at 0° C. The reaction mixture was gradually warmed to ambient temperature and stirred for 3 h. The reaction mixture was concentrated under reduced pressure. The reaction mixture was partitioned between EtOAc and water. The organic layer was collected, washed with water, brine, dried over anhydrous Na₂SO₄, filtered, and concentrated under reduced pressure. The product was purified via flash chromatography to afford (R)-2-(3-nitrophenoxy)-1-(piperidin-1-yl)propan-1-one (q18, 100 mg). LCMS (ESI) Calcd. for C₁₄H₁₈N₂O₄: 278, found [M+H]⁺=280.

Synthesis of (R)-2-(3-aminophenoxy)-1-(piperidin-1-yl)propan-1-one, q19 [Step 4]: To a suspension of (R)-2-(3-nitrophenoxy)-1-(piperidin-1-yl)propan-1-one (q18, 600 mg, 2.2 mmol), Zn dust (560 mg, 8.6 mmol) and NH₄Cl (1.1 g, 21.6 mmol) in ethanol (5 mL) was added water (2.5 mL). The reaction mixture was vigorously stirred at 80° C. for 3 h. The reaction mixture was cooled to ambient temperature and concentrated under reduced pressure. The reaction mixture was extracted with 4% MeOH in CH₂Cl₂ (×3). The combined organic layers were dried over anhydrous Na₂SO₄, filtered, and concentrated under reduced pressure. The product was purified via flash chromatography to afford (R)-2-(3-aminophenoxy)-1-(piperidin-1-yl)propan-1-one (q19, 400 mg). LCMS (ESI) Calcd. for C₁₄H₂₀N₂O₂: 248, found [M+H]⁺=249. ¹H NMR (400 MHz, DMSO-d₆) δ 6.86 (t, 1H), 6.14 (d, 1H), 6.05 (br s, 1H), 6.00 (d, 1H), 5.02-4.98 (m, 3H), 3.48 (br s, 2H), 3.41 (br s, 2H), 1.56-1.47 (m, 6H), 1.36 (d, 3H).

Synthesis of (R)-3-(2-chloro-4-fluorophenyl)-3-oxo-N-(3-((1-oxo-1-(piperidin-1-yl)propan-2-yl)oxy)phenyl)propenamide, q20 [Step 5]: A solution of ethyl 3-(2-chloro-4-fluorophenyl)-3-oxopropanoate (100 mg, 0.4 mmol) and (R)-2-(3-aminophenoxy)-1-(piperidin-1-yl)propan-1-one (q19, 100 mg, 0.4 mmol) in toluene (2 mL) was heated at 110° C. for 12 h. The reaction mixture was cooled to ambient temperature and concentrated under reduced pressure. The product was purified via flash chromatography to afford (R)-3-(2-chloro-4-fluorophenyl)-3-oxo-N-(3-((1-oxo-1-(piperidin-1-yl)propan-2-yl)oxy)phenyl)propanamide (q20, 90 mg). The product was isolated as a mixture corresponding to two compounds of identical masses. LCMS (ESI) Calcd. for C₂₃H₂₄ClFN₂O₄: 446, found [M+H]⁺=447. The mixture of compounds was used in subsequent steps.

Synthesis of (R)-4-(2-chloro-4-fluorophenyl)-7-((1-oxo-1-(piperidin-1-yl)propan-2-yl)oxy)quinolin-2(1H)-one, Example Q5 [Step 6]: A mixture of (R)-3-(2-chloro-4-fluorophenyl)-3-oxo-N-(3-((1-oxo-1-(piperidin-1-yl)propan-2-yl)oxy)phenyl)propanamide (q20, 90 mg, 0.2 mmol) and trifluoroacetic acid (0.15 mL, 2.0 mmol) was stirred at 70° C. for 2 h. The reaction mixture was cooled to ambient temperature, quenched with crushed ice, basified with a saturated aq. NaHCO₃ solution, and extracted with CH₂C₂ (×3). The combined organic layers were washed with brine, dried over anhydrous Na₂SO₄, filtered, and concentrated under reduced pressure. The product was purified via flash chromatography. The product was further purified by reverse-phase prep-HPLC to afford (R)-4-(2-chloro-4-fluorophenyl)-7-((1-oxo-1-(piperidin-1-yl)propan-2-yl)oxy)quinolin-2(1H)-one (Example Q5, 8.0 mg). LCMS (ESI) Calcd. for C₂₃H₂₂ClFN₂O₃: 428, found [M+H]⁺=429. ¹H NMR (400 MHz, DMSO-d₆) (at 100° C.) δ 11.53 (br s, 1H), 7.53 (d, 1H), 7.45 (t, 1H), 7.33 (t, 1H), 6.88 (d, 1H), 6.80 (s, 1H), 6.68 (d, 1H), 6.19 (s, 1H), 5.20 (d, 1H), 3.49 (s, 4H), 1.62-1.47 (m, 9H).

### Example Q6: Synthesis of (R)-2-((4-(2-chloro-4-fluorophenyl)-2-oxo-1,2-dihydroquinolin-7-yl)oxy)-N,N-dimethylpropanamide

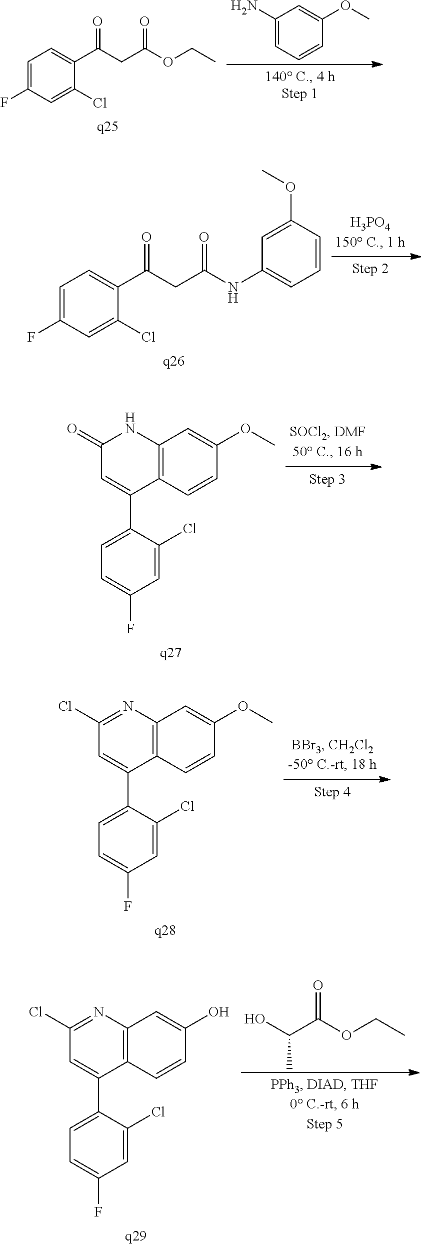

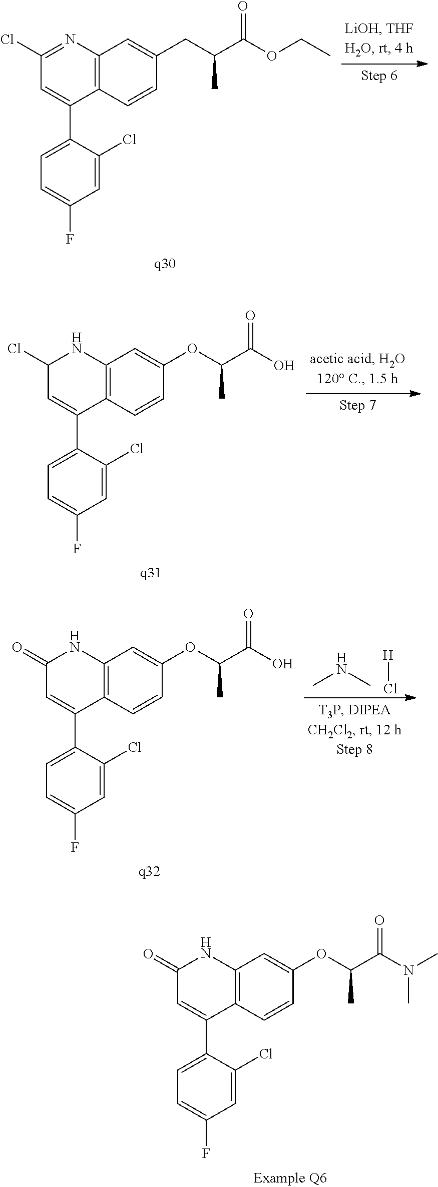

Synthesis of 3-(2-chloro-4-fluorophenyl)-N-(3-methoxyphenyl)-3-oxopropanamide, q26 [Step 1]: A mixture of ethyl 3-(2-chloro-4-fluorophenyl)-3-oxopropanoate (q25, 1.0 g, 4.1 mmol) and 3-methoxyaniline (500 mg, 4.1 mmol) and was heated at 140° C. for 4 h. The reaction mixture was allowed to cool to ambient temperature, diluted with dioxane and 10% aq. HCl, and stirred for 15 min. The reaction mixture was diluted with water and extracted with EtOAc. The combined organic layers were dried over anhydrous Na₂SO₄ and concentrated under reduced pressure. The product was purified by flash chromatography over silica gel to afford 3-(2-chloro-4-fluorophenyl)-N-(3-methoxyphenyl)-3-oxopropanamide (q26, 1.0 g). LCMS (ESI) Calcd. for C₁₆H₁₃ClFNO₃: 321, found [M+H]⁺=322.

Synthesis of 4-(2-chloro-4-fluorophenyl)-7-methoxyquinolin-2(1H)-one, q27 [Step 2]: A mixture of 3-(2-chloro-4-fluorophenyl)-N-(3-methoxyphenyl)-3-oxopropanamide (q26, 2.8 g, 8.6 mmol) and H₃PO₄ (13 mL) was heated at 150° C. for 1 h. The reaction mixture was allowed to cool to ambient temperature and quenched with a 2N aq. solution of NaOH. The reaction mixture was filtered and washed with water and a solution of 5% MeOH in CH₂Cl₂. The reaction mixture was filtered to afford 4-(2-chloro-4-fluorophenyl)-7-methoxyquinolin-2(1H)-one (q27, 920 mg). ¹H NMR (400 MHz, CDCl₃) δ 11.68 (s, 1H), 7.31-7.25 (m, 2H), 7.12 (d, 1H), 7.00 (d, 1H), 6.84 (br s, 1H), 6.73 (d, 1H), 6.45 (s, 1H), 3.90 (s, 3H).

Synthesis of 2-chloro-4-(2-chloro-4-fluorophenyl)-7-methoxyquinoline, q28 [Step 3]: To a stirred solution of 4-(2-chloro-4-fluorophenyl)-7-methoxyquinolin-2(1H)-one (q27, 450 mg, 1.5 mmol) in DMF (0.1 mL, 1.5 mmol) was added SOCl₂ (5.4 mL, 74.1 mmol). The reaction mixture was heated at 50° C. for 16 h. The reaction mixture was concentrated under reduced pressure. The reaction mixture was quenched with cold water and extracted with EtOAc. The combined organic layer was thoroughly washed with water, brine, dried over anhydrous Na₂SO₄, filtered, and concentrated under reduced pressure. The product was purified by flash chromatography over silica gel to afford 2-chloro-4-(2-chloro-4-fluoro-phenyl)-7-methoxy-quinoline (q28, 280 mg). LCMS (ESI) Calcd. for C₁₆H₁₀Cl₂FNO: 321, found [M+H]⁺=322. ¹H NMR (400 MHz, CDCl₃) δ 7.41 (d, 1H), 7.33-7.28 (m, 3H), 7.16-7.11 (m, 3H), 3.94 (s, 3H).

Synthesis of 2-chloro-4-(2-chloro-4-fluorophenyl)quinolin-7-ol, q29 [Step 4]: To a stirred solution of 2-chloro-4-(2-chloro-4-fluoro-phenyl)-7-methoxy-quinoline (q28, 80 mg, 0.3 mmol) in CH₂Cl₂ (1 mL) was added BBr₃ (1.2 mL, 1.2 mmol, 1M in CH₂Cl₂). The reaction mixture was allowed to stir at ambient temperature for 16 h. The reaction mixture was quenched with ice water and extracted with CH₂Cl₂. The organic phase was collected, washed with water, brine, dried over anhydrous Na₂SO₄, filtered, and concentrated under reduced pressure. The product was purified by flash chromatography to afford 2-chloro-4-(2-chloro-4-fluorophenyl)quinolin-7-ol (q29, 60 mg). LCMS (ESI) Calcd. for C₁₅H₈Cl₂FNO: 307, found [M+H]⁺=308. ¹H NMR (400 MHz, DMSO-d₆) δ 10.54 (s, 1H), 7.70 (dd, 1H), 7.56 (dd, 1H), 7.45-7.40 (m, 1H), 7.29 (s, 1H), 7.27-7.25 (m, 2H), 7.16 (dd, 1H).

Synthesis of ethyl (R)-2-((2-chloro-4-(2-chloro-4-fluorophenyl)quinolin-7-yl)oxy)propanoate, q30 [Step 5]: To a stirred solution of 2-chloro-4-(2-chloro-4-fluorophenyl)quinolin-7-ol (q29, 200 mg, 0.6 mmol) in dry THF (3 mL) was added ethyl (S)-2-hydroxypropanoate (120 mg, 1 mmol), PPh₃ (510 mg, 2 mmol) and DIAD (0.4 mL, 2 mmol). The reaction mixture was stirred at ambient temperature for 16 h. The reaction mixture was quenched with water and extracted with EtOAc. The organic phase was collected, washed with water, brine, dried over anhydrous Na₂SO₄, filtered, and concentrated under reduced pressure. The product was purified by flash chromatography over silica gel to afford ethyl (R)-2-((2-chloro-4-(2-chloro-4-fluorophenyl)quinolin-7-yl)oxy)propanoate (q30, 180 mg). LCMS (ESI) Calcd. for C₂₀H₁₆Cl₂FNO₃: 407, found [M+H]⁺=408. ¹H NMR (400 MHz, DMSO-d₆) δ 7.74-7.71 (m, 1H), 7.61-7.56 (m, 1H), 7.47-7.42 (m, 2H), 7.34-7.29 (m, 3H), 5.27-5.25 (m, 1H), 4.21-4.16 (m, 2H), 1.59 (d, 3H), 1.19 (t, 3H).

Synthesis of (2R)-2-((2-chloro-4-(2-chloro-4-fluorophenyl)-1,2-dihydroquinolin-7-yl)oxy)propanoic acid, q31 [Step 6]: To a stirred solution of ethyl (R)-2-((2-chloro-4-(2-chloro-4-fluorophenyl)quinolin-7-yl)oxy)propanoate (q30, 600 mg, 1.5 mmol) in THF (4 mL) and water (1 mL) was added LiOH·H₂O (185 mg, 4.4 mmol). The reaction mixture was stirred at ambient temperature for 4 h. The reaction mixture was concentrated under reduced pressure. The reaction mixture was dissolved in water and extracted with EtOAc. The aqueous layer was separated, acidified with citric acid, and extracted with EtOAc. The combined organic layers were washed with brine, dried over anhydrous Na₂SO₄, filtered, and concentrated under reduced pressure to afford (2R)-2-((2-chloro-4-(2-chloro-4-fluorophenyl)-1,2-dihydroquinolin-7-yl)oxy)propanoic acid (q31, 400 mg). LCMS (ESI) Calcd. for C₁₈H₁₂Cl₂FNO₃: 379, found [M+H]⁺=381.

Synthesis of (R)-2-((4-(2-chloro-4-fluorophenyl)-2-oxo-1,2-dihydroquinolin-7-yl)oxy)propanoic acid, q32 [Step 7]: To a stirred solution of (2R)-2-((2-chloro-4-(2-chloro-4-fluorophenyl)-1,2-dihydroquinolin-7-yl)oxy)propanoic acid (q31, 140 mg, 0.4 mmol) in acetic acid (3.2 mL, 55 mmol) was added water (0.7 mL, 37 mmol). The reaction mixture was heated to reflux for 6 h. The reaction mixture was concentrated under reduced pressure. The reaction mixture was diluted in water and extracted with EtOAc. The combined organic layers were washed with brine, dried over anhydrous Na₂SO₄, filtered, and concentrated under reduced pressure to afford the (R)-2-((4-(2-chloro-4-fluorophenyl)-2-oxo-1,2-dihydroquinolin-7-yl)oxy)propanoic acid (q32, 90 mg). LCMS (ESI) Calcd. for C₁₈H₁₃ClFNO₄: 361, found [M+H]⁺=362. ¹H NMR (400 MHz, DMSO-d₆) δ 11.75 (s, 1H), 7.64 (dd, 1H), 7.49-7.45 (m, 1H), 7.40-7.35 (m, 1H), 6.79-6.78 (m, 2H), 6.63 (dd, 1H), 6.14 (s, 1H), 4.49 (d, 1H), 1.41 (d, 3H).

Synthesis of (R)-2-((4-(2-chloro-4-fluorophenyl)-2-oxo-1,2-dihydroquinolin-7-yl)oxy)-N,N-dimethylpropanamide, Example Q6 [Step 8]: To a stirred solution (R)-2-((4-(2-chloro-4-fluorophenyl)-2-oxo-1,2-dihydroquinolin-7-yl)oxy)propanoic acid (q32, 90 mg, 0.3 mmol) in CH₂Cl₂ (3 mL) was added N-methylmethanamine.HCl (81 mg, 1.0 mmol) followed by DIPEA (0.2 mL, 1.2 mmol) and T₃P (0.2 mL, 0.4 mmol). The reaction mixture was allowed to stir at ambient temperature for 16 h. The reaction mixture was concentrated under reduced pressure. The product was purified by reverse phase prep-HPLC to afford (R)-2-((4-(2-chloro-4-fluorophenyl)-2-oxo-1,2-dihydroquinolin-7-yl)oxy)-N,N-dimethylpropanamide (Example Q6, 52 mg). LCMS (ESI) Calcd. for C₂₀H₁₈ClFN₂O₃: 388, found [M+H]⁺=389. ¹H NMR (400 MHz, DMSO-d₆) δ 11.89 (s, 1H), 7.64 (dd, 1H), 7.50-7.47 (m, 1H), 7.41-7.36 (m, 1H), 6.84 (d, 1H), 6.67-6.64 (m, 2H), 6.20 (s, 1H), 5.25-5.22 (m, 1H), 3.12 (s, 3H), 2.85 (s, 3H) 1.44 (d, 3H).

### Examples Q7-Q8: Synthesis of chiral analogs of N,N-dimethyl-2-((2-oxo-4-(o-tolyl)-1,2-dihydroquinolin-7-yl)oxy)propenamide

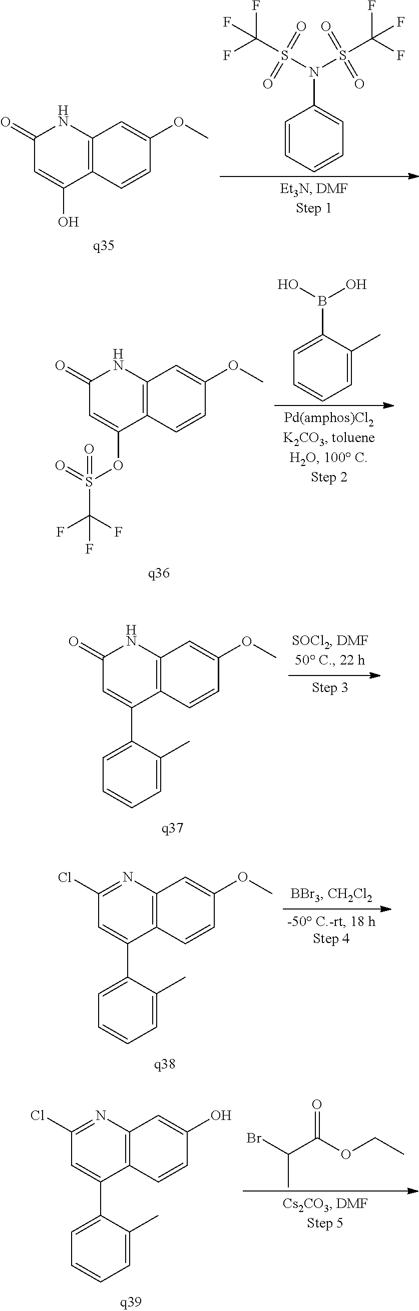

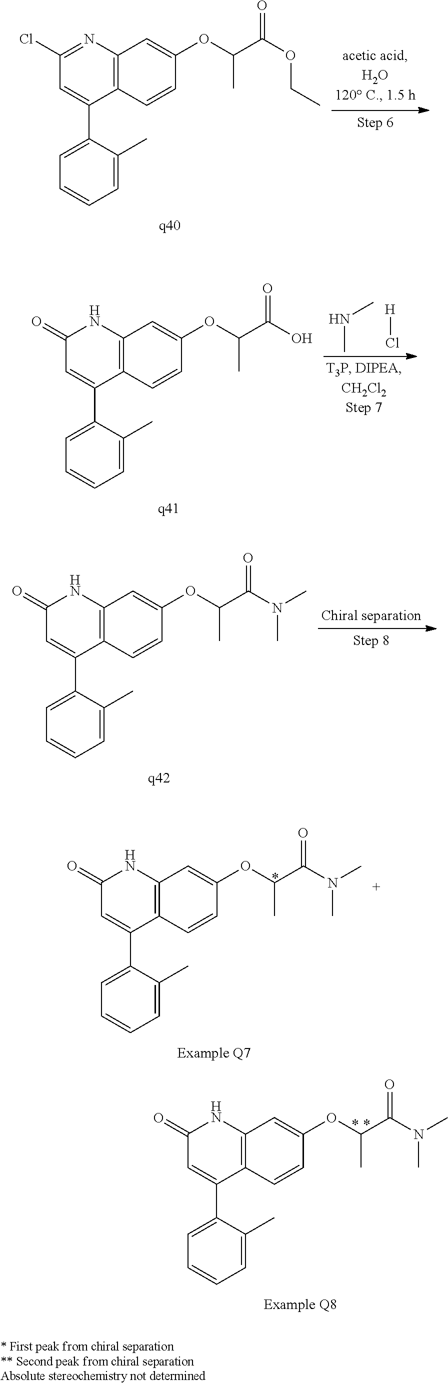

Synthesis of 7-methoxy-2-oxo-1,2-dihydroquinolin-4-yl trifluoromethanesulfonate, q36 [Step 1]: To a suspension of 4-hydroxy-7-methoxyquinolin-2(1H)-one (q35, 1.0 g, 5.2 mmol) in DMF (20 mL) was added triethylamine (2.2 mL, 15.7 mmol) followed by 1,1,1-trifluoro-N-phenyl-N-(trifluoromethylsulfonyl)methanesulfonamide (2.2 g, 6.3 mmol). The reaction mixture was stirred at ambient temperature for 6 h. Water was added, and the reaction mixture was stirred for 30 min. The reaction mixture filtered, washed with water, and dried under reduced pressure to afford 7-methoxy-2-oxo-1,2-dihydroquinolin-4-yl trifluoromethanesulfonate (q36, 900 mg). LCMS (ESI) Calcd. for C₁₁H₈F₃NO₅S: 323, found [M+H]⁺=324. ¹H NMR (400 MHz, DMSO-d₆) δ 12.16 (s, 1H), 7.56 (d, 1H), 7.00 (dd, 1H), 6.92 (m, 1H), 6.51 (s, 1H), 3.85 (s, 3H).

Synthesis of 7-methoxy-4-(o-tolyl)quinolin-2(1H)-one, q37 [Step 2]: To a stirred solution of o-tolylboronic acid (505 mg, 3.7 mmol) and 7-methoxy-2-oxo-1,2-dihydroquinolin-4-yl trifluoromethanesulfonate (q36, 1.0 g, 3 mmol) in toluene (7 mL) was added water (1 mL) in a sealed tube. The reaction mixture was degassed with nitrogen prior to the addition of K₂CO₃ (1.0 g, 7.7 mmol) and Pd(amphos)Cl₂ (220 mg, 0.3 mmol). The reaction mixture was stirred at 110° C. for 16 h. The reaction mixture was diluted with water and extracted with EtOAc (×3). The combined organic layers were washed with brine, dried over anhydrous Na₂SO₄, filtered, and concentrated under reduced pressure. The product was purified by flash column chromatography to afford 7-methoxy-4-(o-tolyl)quinolin-2(1H)-one (q37, 800 mg). LCMS (ESI) Calcd. for C₁₇H₁₅NO₂: 265, found [M+H]⁺=266. ¹H NMR (400 MHz, DMSO-d₆) δ 11.73 (s, 1H), 7.39-7.35 (m, 2H), 7.33-7.23 (m, 1H), 7.22 (d, 1H), 6.89 (d, 1H), 6.82 (d, 1H), 6.72 (dd, 1H), 6.12 (s, 1H), 3.80 (s, 3H), 2.04 (s, 3H).

Synthesis of 2-chloro-7-methoxy-4-(o-tolyl)quinoline, q38 [Step 3]: To a stirred solution of 7-methoxy-4-(o-tolyl)-quinolin-2(1H)-one (q37, 500 mg, 1.9 mmol) in SOCl₂ (6.8 mL, 94.2 mmol) was added DMF (0.2 mL, 1.9 mmol). The reaction mixture was stirred at 50° C. for 12 h. The reaction mixture was concentrated under reduced pressure. The reaction mixture was quenched with a saturated aq. NaHCO₃ solution and extracted with EtOAc (×2). The combined organic layers were washed with brine, dried over anhydrous Na₂SO₄, filtered, and concentrated under reduced pressure. The product was purified by flash column chromatography to afford 2-chloro-7-methoxy-4-(o-tolyl)quinoline (q38, 400 mg). LCMS (ESI) Calcd. for C₁₇H₁₄ClNO: 283, found [M+H]⁺=284. ¹H NMR (400 MHz, DMSO-d₆) δ 7.45-7.40 (m, 3H), 7.37-7.34 (m, 1H), 7.30 (s, 1H), 7.26-7.21 (m, 3H), 3.93 (s, 3H), 1.98 (s, 3H).

Synthesis of 2-chloro-4-(o-tolyl)quinolin-7-ol, q39 [Step 4]: To a stirred solution of 2-chloro-7-methoxy-4-(o-tolyl)quinoline (q38, 400 mg, 1.4 mmol) in CH₂Cl₂ (5 mL) was added dropwise BBr₃ (4.2 mL, 4.2 mmol, 1M in CH₂Cl₂) at 0° C. The reaction mixture was gradually warmed to ambient temperature and stirred for 12 h. The reaction mixture was quenched with MeOH and concentrated under reduced pressure. The product was purified by flash column chromatography to afford 2-chloro-4-(o-tolyl)quinolin-7-ol (q39, 360 mg). LCMS (ESI) Calcd. for C₁₆H₁₂ClNO: 269, found [M+H]⁺=270. ¹H NMR (400 MHz, DMSO-d₆) δ 10.45 (br s, 1H), 7.45-7.39 (m, 2H), 7.34 (t, 1H), 7.24-7.19 (m, 4H), 7.14 (d, 1H), 1.98 (s, 3H).

Synthesis of ethyl 2-((2-chloro-4-(o-tolyl)quinolin-7-yl)oxy)propanoate, q40 [Step 5]: To a stirred solution of 2-chloro-4-(o-tolyl)quinolin-7-ol (q39, 380 mg, 1.4 mmol) in DMF (3 mL) was added Cs₂CO₃ (1.1 g, 3.5 mmol) followed by ethyl 2-bromopropanoate (255 mg, 1.4 mmol). The reaction mixture was stirred at 100° C. for 2 h. The reaction mixture was partitioned between EtOAc and water. The organic layer was collected, washed with water (×4) and brine, dried over anhydrous Na₂SO₄, filtered, and concentrated under reduced pressure. The product was purified by flash column chromatography to afford ethyl 2-((2-chloro-4-(o-tolyl)quinolin-7-yl)oxy)propanoate (q40, 380 mg). LCMS (ESI) Calcd. for C₂₁H₂₀ClNO₃: 369, found [M+H]⁺=370. ¹H NMR (400 MHz, DMSO-d₆) δ 7.45-7.43 (m, 2H), 7.37 (br s, 2H), 7.29-7.22 (m, 4H), 5.24 (br s, 1H), 4.20 (q, 2H), 1.99 (d, 3H), 1.59 (d, 3H), 1.21 (t, 3H).

Synthesis of 2-((2-oxo-4-(o-tolyl)-1,2-dihydroquinolin-7-yl)oxy)propanoic acid, q41 [Step 6]: To a stirred solution of ethyl 2-[[2-chloro-4-(o-tolyl)-7-quinolyl]oxy]propanoate (q40, 380 mg, 1 mmol) in acetic acid (8.8 mL, 154 mmol) was added water (1.8 mL). The reaction mixture was heated to reflux for 6 h. The reaction mixture was concentrated under reduced pressure. The reaction mixture was partitioned between EtOAc and water. The combined organic layers were washed with brine, dried over anhydrous Na₂SO₄, filtered, and concentrated under reduced pressure to afford 2-((2-oxo-4-(o-tolyl)-1,2-dihydroquinolin-7-yl)oxy)propanoic acid (q41, 300 mg). The product was used in the next step without further purification. LCMS (ESI) Calcd. for C₁₉H₁₇NO₄: 323, found [M+H]⁺=324. ¹H NMR (400 MHz, DMSO-d₆) δ 11.74 (s, 1H), 7.43-7.37 (m, 2H), 7.32 (m, 1H), 7.18 (d, 1H), 6.81 (br s, 2H), 6.69 (d, 1H), 6.12 (s, 1H), 4.86-4.81 (m, 2H), 1.98 (d, 3H), 1.58 (d, 3H).

Synthesis of N,N-dimethyl-2-((2-oxo-4-(o-tolyl)-1,2-dihydroquinolin-7-yl)oxy)propenamide, q42 [Step 7]: To a solution of 2-((2-oxo-4-(o-tolyl)-1,2-dihydroquinolin-7-yl)oxy)propanoic acid (q41, 260 mg, 0.8 mmol) and dimethyl amine hydrochloride (78 mg, 1 mmol) in CH₂Cl₂ (5 mL) was added DIPEA (0.27 mL, 2 mmol) and T₃P (665 mg, 1.1 mmol, 50% in EtOAc) dropwise at 0° C. The reaction mixture was stirred for 2 h. The reaction mixture was diluted with CH₂Cl₂, washed with water, brine, dried over anhydrous Na₂SO₄, filtered, and concentrated under reduced pressure. The product was purified via flash chromatography to afford N,N-dimethyl-2-((2-oxo-4-(o-tolyl)-1,2-dihydroquinolin-7-yl)oxy)propanamide (q42, 152 mg).

Synthesis of chiral N,N-dimethyl-2-((2-oxo-4-(o-tolyl)-1,2-dihydroquinolin-7-yl)oxy)propenamide, Examples Q7 and Q8 [Step 8]: N,N-dimethyl-2-((2-oxo-4-(o-tolyl)-1,2-dihydroquinolin-7-yl)oxy)propanamide (q42, 152 mg) was purified by normal phase chiral prep-HPLC and lyophilized. The first product was isolated as N,N-dimethyl-2-((2-oxo-4-(o-tolyl)-1,2-dihydroquinolin-7-yl)oxy)propenamide, Peak 1 (Example Q7, 40 mg) and the second product as N,N-dimethyl-2-((2-oxo-4-(o-tolyl)-1,2-dihydroquinolin-7-yl)oxy)propenamide, Peak 2 (Example Q8, 40 mg). The absolute stereochemistry of these Examples was not determined.

Example Q7: N,N-dimethyl-2-((2-oxo-4-(o-tolyl)-1,2-dihydroquinolin-7-yl)oxy)propenamide, Peak 1: LCMS (ESI) Calcd. for C₂₁H₂₂N₂O₃: 350, found [M+H]+=351. ¹H NMR (400 MHz, DMSO-d₆) δ 11.82 (br s, 1H), 7.40-7.38 (m, 2H), 7.37-7.29 (m, 1H), 7.17 (d, 1H), 6.79 (d, 1H), 6.64-6.61 (m, 2H), 6.12 (s, 1H), 5.23-5.20 (m, 1H), 3.12 (s, 3H), 2.85 (s, 3H), 2.07 (s, 3H), 1.51 (d, 3H).

Example Q8: N,N-dimethyl-2-((2-oxo-4-(o-tolyl)-1,2-dihydroquinolin-7-yl)oxy)propanamide, Peak 2: LCMS (ESI) Calcd. for C₂₁H₂₂N₂O₃: 350, found [M+H]+=351. ¹H NMR (400 MHz, DMSO-d₆) δ 11.82 (br s, 1H), 7.40-7.38 (m, 2H), 7.37-7.29 (m, 1H), 7.17 (d, 1H), 6.79 (d, 1H), 6.64-6.61 (m, 2H), 6.12 (s, 1H), 5.23-5.20 (m, 1H), 3.12 (s, 3H), 2.85 (s, 3H), 2.07 (s, 3H), 1.51 (d, 3H).

Chiral prep-HPLC: Chiral separation was performed on an Agilent 1200 series instrument. Column was a Chiralpak IC (250×21 mm), 5 μm, operating at ambient temperature with flow rate of 21.0 mL/min. Mobile phase: 0.1% isopropylamine in a mixture of 70% hexanes, 15% CH₂Cl₂, and 15% ethanol, held isocratic for up to 15 min. with detection at 232 nm wavelength.

### Example Q9: Synthesis of 7-methoxy-4-phenylquinolin-2(1H)-one

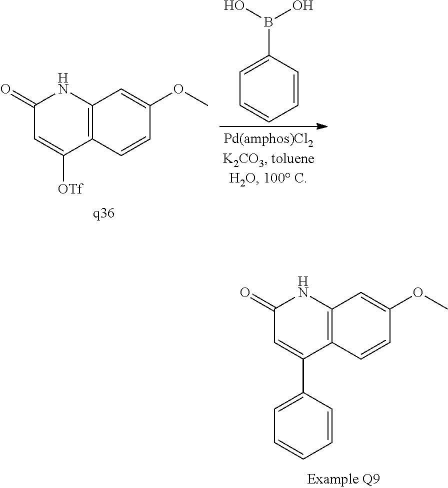

Synthesis of 7-methoxy-4-phenylquinolin-2(1H)-one, Example Q9: To a stirred solution of phenylboronic acid (38 mg, 0.3 mmol) and (7-methoxy-2-oxo-1H-quinolin-4-yl) trifluoromethanesulfonate (q36, 100 mg, 0.3 mmol) in toluene (3.5 mL) and water (0.5 mL) was added K₂CO₃ (107 mg, 0.8 mmol) in sealed tube. The reaction mixture was degassed with argon for 5 min. prior to the addition of Pd(amphos)Cl₂ (22 mg, 0.03 mmol). The reaction mixture was stirred at 100° C. for 12 h. The reaction mixture was diluted with EtOAc, washed with water, brine, dried over anhydrous Na₂SO₄, filtered, and concentrated under reduced pressure. The product was purified by flash column chromatography, further purified by prep-HPLC, and lyophilized to afford 7-methoxy-4-phenylquinolin-2(1H)-one (Example Q9, 30 mg). LCMS (ESI) Calcd. for C₁₆H₁₃NO₂: 251, found [M+H]⁺=252. ¹H NMR (400 MHz, DMSO-d₆) δ 11.73 (s, 1H), 7.54-7.48 (m, 3H), 7.46 (dd, 2H), 7.28 (d, 1H), 6.90 (d, 1H), 6.78 (dd, 1H), 6.20 (s, 1H), 3.81 (s, 3H).

### Examples Q10-Q11: Synthesis of 7-isopropoxy-4-(o-tolyl)quinolin-2(1H)-one and 7-(sec-butoxy)-4-(o-tolyl)quinolin-2(1H)-one

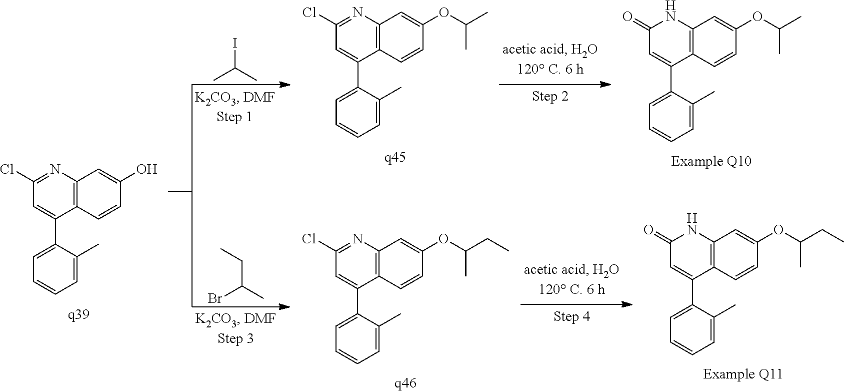

Synthesis of 2-chloro-7-isopropoxy-4-(o-tolyl)quinoline, q45 [Step 1]: To a stirred solution of 2-chloro-4-(o-tolyl)quinolin-7-ol (q39, 190 mg, 0.7 mmol) in DMF (3 mL) was added K₂CO₃ (245 mg, 1.7 mmol) followed by 2-iodopropane (155 mg, 0.9 mmol). The reaction mixture was heated at 100° C. for 2 h. The reaction mixture was diluted with EtOAc, washed with water, brine, dried over anhydrous Na₂SO₄, filtered, and concentrated under reduced pressure. The product was purified by flash column chromatography to afford 2-chloro-7-isopropoxy-4-(o-tolyl)quinoline (q45, 180 mg). LCMS (ESI) Calcd. for C₁₉H₁₈ClNO: 311, found [M+H]⁺=312. ¹H NMR (400 MHz, DMSO-d₆) δ 7.46-7.40 (m, 3H), 7.37-7.34 (m, 1H), 7.28 (s, 1H), 7.25 (d, 2H), 7.20 (dd, 1H), 4.89-4.85 (m, 1H), 1.99 (s, 3H), 1.35 (d, 6H).

Synthesis of 7-isopropoxy-4-(o-tolyl)quinolin-2(1H)-one, Example Q10 [Step 2]: To a stirred solution of 2-chloro-7-isopropoxy-4-(o-tolyl)quinoline (q45, 200 mg, 0.6 mmol) in acetic acid (5.5 mL, 96.2 mmol) was added water (1.2 mL, 64.1 mmol). The reaction mixture was heated to reflux for 6 h. The reaction mixture was concentrated under reduced pressure. The product was purified by reverse phase prep-HPLC and lyophilized to afford 7-isopropoxy-4-(o-tolyl)quinolin-2(1H)-one (65 mg). LCMS (ESI) Calcd. for C₁₉H₁₉NO₂: 293, found [M+H]⁺=294. ¹H NMR (400 MHz, DMSO-d₆) δ 11.65 (s, 1H), 7.38-7.35 (m, 2H), 7.33-7.29 (m, 1H), 7.18 (d, 1H), 6.87 (d, 1H), 6.80 (d, 1H), 6.69 (dd, 1H), 6.10 (s, 1H), 4.62-4.59 (m, 1H), 2.05 (s, 3H), 1.30 (d, 6H).

Synthesis of 7-(sec-butoxy)-2-chloro-4-(o-tolyl)quinoline, q46 [Step 3]: To a stirred solution of 2-chloro-4-(o-tolyl)quinolin-7-ol (q39, 100 mg, 0.4 mmol) in DMF (3 mL) was added K₂CO₃ (130 mg, 0.9 mmol) followed by 2-bromobutane (65 mg, 0.5 mmol). The reaction mixture was heated at 100° C. for 2 h. The reaction mixture was partitioned between EtOAc and water. The organic layer was collected, washed with water, brine, dried over anhydrous Na₂SO₄, filtered, and concentrated under reduced pressure. The product was purified by flash column chromatography to afford 7-(sec-butoxy)-2-chloro-4-(o-tolyl)quinoline (q46, 100 mg). LCMS (ESI) Calcd. for C₂₀H₂₀ClNO: 325, found [M+H]⁺=326. ¹H NMR (400 MHz, DMSO-d₆) δ 7.44-7.42 (m, 3H), 7.33 (t, 1H), 7.27-7.18 (m, 4H), 4.65-4.62 (m, 1H), 1.99 (s, 3H), 1.75-1.61 (m, 2H), 1.31 (d, 3H), 0.96 (t, 3H).

Synthesis of 7-(sec-butoxy)-4-(o-tolyl)quinolin-2(1H)-one, Example Q11 [Step 4]: To a stirred solution of 7-(sec-butoxy)-2-chloro-4-(o-tolyl)quinoline (q46, 100 mg, 0.3 mmol) in acetic acid (2.6 mL, 46.0 mmol) was added water (0.6 mL, 30.7 mmol). The reaction mixture was heated to reflux for 6 h. The reaction mixture was concentrated under reduced pressure. The product was purified by reverse phase prep-HPLC and lyophilized to afford 7-(sec-butoxy)-4-(o-tolyl)quinolin-2(1H)-one (Example Q11, 54 mg). LCMS (ESI) Calcd. for C₂₀H₂₁NO₂: 307, found [M+H]⁺=308. ¹H NMR (400 MHz, DMSO-d₆) δ 11.66 (s, 1H), 7.40-7.35 (m, 2H), 7.33 (t, 1H), 7.18-7.16 (d, 1H), 6.87 (d, 1H), 6.80 (d, 1H), 6.70 (dd, 1H), 6.10 (d, 1H), 4.41-4.33 (m, 1H), 2.05 (s, 3H), 1.71-1.56 (m, 2H), 1.26 (d, 3H), 0.94 (t, 3H).

### Example Q12: Synthesis of 7-isobutoxy-4-(o-tolyl)quinolin-2(1H)-one

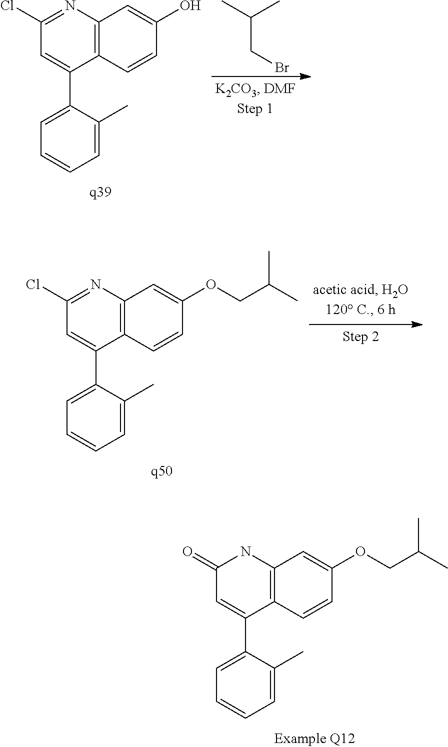

Synthesis of 2-chloro-7-isobutoxy-4-(o-tolyl)quinoline, q50 [Step 1]: To a stirred solution of 2-chloro-4-(o-tolyl)quinolin-7-ol (q39, 360 mg, 1.3 mmol) in DMF (3 mL) was added K₂CO₃ (460 mg, 3.3 mmol) followed by 1-bromo-2-methyl-propane (240 mg, 1.7 mmol). The reaction mixture was heated to 100° C. for 2 h. The reaction mixture was partitioned between EtOAc and water. The organic layer was collected, washed with cold water, brine, dried over anhydrous Na₂SO₄, filtered, and concentrated under reduced pressure. The product was purified by flash column chromatography to afford 2-chloro-7-isobutoxy-4-(o-tolyl)quinoline (q50, 350 mg). ¹H NMR (400 MHz, DMSO-d₆) δ 7.46-7.41 (m, 3H), 7.37 (t, 1H), 7.29 (s, 1H), 7.26 (s, 3H), 3.97 (d, 2H), 2.14-2.00 (m, 1H), 1.98 (s, 3H), 1.02 (d, 6H).

Synthesis of 7-isobutoxy-4-(o-tolyl)quinolin-2(1H)-one, Example Q12 [Step 2]: To a stirred solution of 2-chloro-7-isobutoxy-4-(o-tolyl)quinoline (q50, 300 mg, 0.9 mmol) in acetic acid (7.9 mL, 138 mmol) was added water (1.7 mL, 92.1 mmol). The reaction mixture was heated to reflux for 6 h. The reaction mixture was concentrated under reduced pressure. The product was purified by reverse phase prep-HPLC and lyophilized to afford 7-isobutoxy-4-(o-tolyl)quinolin-2(1H)-one (Example Q12, 150 mg). LCMS (ESI) Calcd. for C₂₀H₂₁NO₂: 307, found [M+H]⁺: 309. ¹H NMR (400 MHz, DMSO-d₆) δ 11.70 (s, 1H), 7.38-7.33 (m, 2H), 7.31-7.29 (m, 1H), 7.18 (d, 1H), 6.87 (d, 1H), 6.81 (d, 1H), 6.73 (dd, 1H), 6.11 (s, 1H), 3.80 (d, 2H), 2.08-2.00 (s, 4H), 0.99 (d, 6H).

### Example Q13-Q15: Synthesis of (R)-2-((2-oxo-4-(o-tolyl)-1,2-dihydroquinolin-7-yl)oxy)propanoic acid, (R)-1-(2-((2-oxo-4-(o-tolyl)-1,2-dihydroquinolin-7-yl)oxy)propanoyl)piperidine-4-carboxylic acid, and (S)-1-((R)-2-((2-oxo-4-(o-tolyl)-1,2-dihydroquinolin-7-yl)oxy)propanoyl)piperidine-3-carboxylic acid

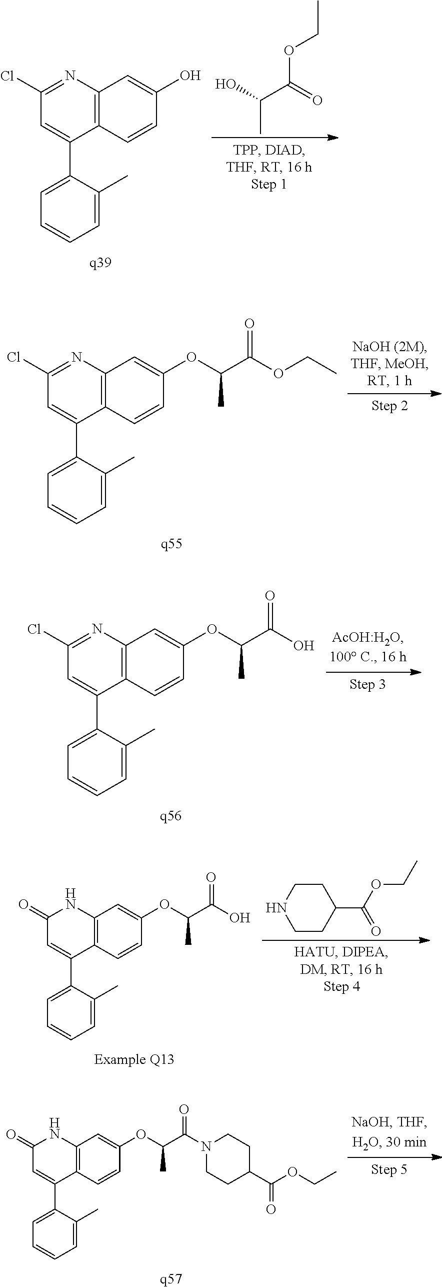

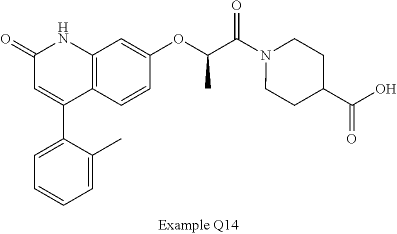 [SMILES: Cc1ccccc1-c1cc(=O)[nH]c2cc(O[C@H](C)C(=O)N3CCC(C(=O)O)CC3)ccc12]

Synthesis of ethyl (R)-2-((2-chloro-4-(o-tolyl)quinolin-7-yl)oxy)propanoate, q55 [Step 1]: To a stirred solution of 2-chloro-4-(o-tolyl)quinolin-7-ol (q39, 2.40 g, 8.90 mmol) in tetrahydrofuran (20 mL), was added ethyl (2S)-2-hydroxypropanoate (1.5 mL, 13.3 mmol), triphenyl phosphine (3.50 g, 13.3 mmol), and DIAD at 0° C. and the reaction mixture was stirred at ambient temperature for 16 h. under an inert atmosphere. The reaction mixture was concentrated under reduced pressure and the product was purified by flash chromatography to afford ethyl (2R)-2-[[2-chloro-4-(o-tolyl)-7-quinolyl]oxy]propanoate (q55, 2.9 g). LCMS (ESI) Calcd. C₂₁H₂₀ClNO₃ for 369, found [M+H]⁺=370. ¹H NMR (400 MHz, DMSO-d₆) δ 7.47-7.41 (m, 2H), 7.38-7.33 (m, 2H), 7.29-7.23 (m, 4H), 5.26-5.21 (m, 1H), 4.20-4.15 (m, 2H), 1.99 (s, 3H), 1.58 (d, 3H), 1.21 (m, 3H).

Synthesis of (R)-2-((2-chloro-4-(o-tolyl)quinolin-7-yl)oxy)propanoic acid, q56 [Step 2]: To a stirred solution of ethyl (2R)-2-[[2-chloro-4-(o-tolyl)-7-quinolyl]oxy]propanoate (q55, 2.90 g, 7.84 mmol) in tetrahydrofuran (10 mL) and methanol (10 mL), was added an aqueous NaOH solution (2M) (1.57 g, 39.2 mmol) at 25° C. and the reaction mixture was stirred at ambient temperature for 1 h. The reaction mixture was concentrated under reduced pressure and the residual aqueous fraction was acidified to pH-3 using a 1N HCl and extracted with a mixture of methanol in dichloromethane (1:9) (thrice). The combined organic layer was dried over anhydrous Na₂SO₄ and concentrated under reduced pressure to afford (R)-2-[[2-chloro-4-(o-tolyl)-7-quinolyl]oxy]propanoic acid (q56, 2.32 g). LCMS (ESI) Calcd. C₁₉H₁₆ClNO₃ for 341, found [M+H]⁺=342. ¹H NMR (400 MHz, DMSO-d₆) δ 13.20 (br s, 1H), 7.46-7.42 (m, 2H), 7.37-7.32 (m, 2H), 7.27-7.22 (m, 4H), 5.12-5.07 (m, 1H), 2.00 (s, 3H), 1.58 (d, 3H).

Synthesis of (R)-2-((2-oxo-4-(o-tolyl)-1,2-dihydroquinolin-7-yl)oxy)propanoic acid, Example Q13 [Step 3]: A solution (2R)-2-[[2-chloro-4-(o-tolyl)-7-quinolyl]oxy]propanoic acid (q56, 2.90 g, 8.48 mmol) in acetic acid (33 mL) and water (3 mL) was heated at 100° C. for 16 h. in a sealed tube. The reaction mixture was diluted with ethyl acetate and washed with water, brine, dried over anhydrous Na₂SO₄ and concentrated under reduced pressure to give (2R)-2-[[4-(o-tolyl)-2-oxo-1H-quinolin-7-yl]oxy]propanoic acid (Example Q13, 1.30 g). LCMS (ESI) Calcd. for C₁₉H₁₇NO₄: 323, found [M+H]⁺=324. ¹H NMR (400 MHz, DMSO-d₆): δ11.70 (br s, 1H), 7.38-7.31 (m, 4H), 7.17-7.16 (m, 1H), 6.69-6.74 (m, 2H), 6.64-6.62 (m, 1H), 6.08 (s, 1H), 4.61-4.59 (m, 1H), 2.05 (d, 3H), 1.45 (d, 3H).

Synthesis of ethyl (R)-1-(2-((2-oxo-4-(o-tolyl)-1,2-dihydroquinolin-7-yl)oxy)propanoyl)piperidine-4-carboxylate, q57 [Step 4]: To a stirred solution of (R)-2-((2-oxo-4-(o-tolyl)-1,2-dihydroquinolin-7-yl)oxy)propanoic acid (Example Q13, 300 mg, 0.9 mmol) and ethyl piperidine-4-carboxylate (212 mg, 1.35 mmol) in dimethylformamide (3 mL), was added DIPEA (0.8 mL, 4.5 mmol) and the reaction mixture was stirred at 25° C. for 5 min. To the reaction mixture was added HATU (0.53 mg, 4.5 mmol) and stirring was continued at ambient temperature for 16 h. The reaction mixture was diluted with ethyl acetate, washed with water, brine, dried over anhydrous Na₂SO₄ and concentrated under reduced pressure. The product was purified by flash chromatography to afford (R)-1-(2-((2-oxo-4-(o-tolyl)-1,2-dihydroquinolin-7-yl)oxy)propanoyl)piperidine-4-carboxylate (q57, 210 mg). LCMS (ESI) Calcd. for C₂₇H₃₀N₂O₅: 462, found [M+H]⁺=463.

Synthesis of (R)-1-(2-((2-oxo-4-(o-tolyl)-1,2-dihydroquinolin-7-yl)oxy)propanoyl)piperidine-4-carboxylic acid, Example Q14 [Step 5]: To a stirred solution of (R)-1-(2-((2-oxo-4-(o-tolyl)-1,2-dihydroquinolin-7-yl)oxy)propanoyl)piperidine-4-carboxylate (q57, 210 mg, 0.45 mmol) in tetrahydrofuran (8 mL) and methanol (2 mL) was added aqueous NaOH (2M) (91 mg, 2.27 mmol) at 0° C., and the reaction mixture was stirred at ambient temperature for 30 min. The reaction mixture was concentrated under reduced pressure and the residual aqueous fraction was acidified to pH-3 using 1N HCl (aqueous) and extracted with a mixture of methanol in dichloromethane (1:9) (thrice). The combined organic layer was dried over anhydrous Na₂SO₄ and concentrated under reduced pressure. The product was purified by prep-HPLC to afford (R)-1-(2-((2-oxo-4-(o-tolyl)-1,2-dihydroquinolin-7-yl)oxy)propanoyl)piperidine-4-carboxylic acid (Example Q14, 46 mg). LCMS (ESI) Calcd. for C₂₅H₂₆N₂O₅: 434, found [M+H]⁺=435. ¹H NMR (400 MHz, DMSO-d₆): δ 11.86 (s, 1H), 7.41-7.37 (m, 2H), 7.33-7.20 (m, 1H), 7.18-7.16 (m, 1H), 6.80-6.79 (m, 1H), 6.69-6.65 (m, 2H), 6.12 (s, 1H), 5.27 (br s, 1H), 4.19-4.07 (m, 1H), 3.97-3.88 (m, 1H), 3.21-3.18 (m, 1H), 2.83-2.79 (m, 1H) 2.05 (s, 3H), 1.91-1.83 (m, 3H), 1.43-1.33 (m, 5H).

### Example Q15: Synthesis of (S)-1-((R)-2-((2-oxo-4-(o-tolyl)-1,2-dihydroquinolin-7-yl)oxy)propanoyl)piperidine-3-carboxylic acid

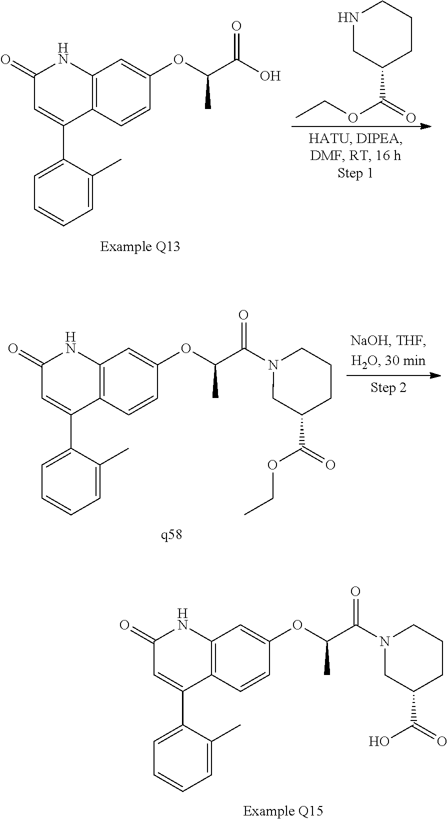 [SMILES: Cc1ccccc1-c1cc(=O)[nH]c2cc(O[C@H](C)C(=O)O)ccc12 ; CCOC(=O)[C@H]1CCCNC1 ; CCOC(=O)[C@H]1CCCN(C(=O)[C@@H](C)Oc2ccc3c(-c4ccccc4C)cc(=O)[nH]c3c2)C1 ; Cc1ccccc1-c1cc(=O)[nH]c2cc(O[C@H](C)C(=O)N3CCC[C@H](C(=O)O)C3)ccc12]

Synthesis of ethyl (S)-1-((R)-2-((2-oxo-4-(o-tolyl)-1,2-dihydroquinolin-7-yl)oxy)propanoyl)piperidine-3-carboxylate, q58 [Step 1]: To a stirred solution of (R)-2-((2-oxo-4-(o-tolyl)-1,2-dihydroquinolin-7-yl)oxy)propanoic acid (Example Q13, 300 mg, 0.9 mmol) and ethyl (S)-piperidine-3-carboxylate (212 mg, 1.35 mmol) in dimethylformamide (3 mL), was added DIPEA (0.8 mL, 4.5 mmol) and the reaction mixture was stirred at 25° C. for 5 min. To the reaction mixture was added HATU (0.53 mg, 4.5 mmol) and it was stirred at ambient temperature for 16 h. The reaction mixture was diluted with ethyl acetate, washed with water, brine, dried over anhydrous Na₂SO₄ and concentrated under reduced pressure. The product was purified by flash chromatography to afford ethyl (S)-1-((R)-2-((2-oxo-4-(o-tolyl)-1,2-dihydroquinolin-7-yl)oxy)propanoyl)piperidine-3-carboxylate (q58, 350 mg). LCMS (ESI) Calcd. for C₂₇H₃₀N₂O₅: 462, found [M+H]⁺=463.

Synthesis of (S)-1-((R)-2-((2-oxo-4-(o-tolyl)-1,2-dihydroquinolin-7-yl)oxy)propanoyl)piperidine-3-carboxylic acid, Example Q15 [Step 2]: To a stirred solution of ethyl (S)-1-((R)-2-((2-oxo-4-(o-tolyl)-1,2-dihydroquinolin-7-yl)oxy)propanoyl)piperidine-3-carboxylate (q58, 200 mg, 0.43 mmol) in tetrahydrofuran (8 mL) and methanol (2 mL) was added aqueous NaOH (2M) (91 mg, 2.27 mmol) at 0° C. and stirred at ambient temperature for 30 min. The reaction mixture was concentrated under reduced pressure and the residual aqueous fraction was acidified to pH-3 using 1N HCl. The aqueous fraction was extracted with a mixture of methanol in dichloromethane (1:9) (thrice). The combined organic extract was dried over anhydrous Na₂SO₄ and concentrated under reduced pressure. The product was purified by prep-HPLC and lyophilized to afford (S)-1-((R)-2-((2-oxo-4-(o-tolyl)-1,2-dihydroquinolin-7-yl)oxy)propanoyl)piperidine-3-carboxylic acid (Example Q15, 70 mg). LCMS (ESI) Calcd. for C₂₅H₂₆N₂O₅: 434, found [M+H]⁺=435. ¹H NMR (400 MHz, DMSO-d₆) (at 100° C.): δ 11.94 (br s, 1H), 11.50 (br s, 1H), 7.40-7.30 (m, 3H), 7.17 (d, 1H), 6.84-6.79 (m, 2H), 6.65 (d, 1H), 6.11 (s, 1H), 5.25-5.24 (m, 1H), 4.10-4.06 (m, 1H), 3.85-3.82 (m, 1H), 3.10-3.02 (m, 1H), 2.67-2.50 (m, 1H), 2.40-2.32 (m, 1H), 2.08 (s, 3H), 1.97 (m, 1H), 1.67 (m, 2H), 1.48-1.47 (m, 4H).

### Biological/Biochemical Evaluation

### General Protocol for In Vitro Analysis of Compounds

The inhibitory activity of the compounds of the present invention against POLRMT were determined by assays based on Bergbrede, T., et al., “An adaptable high-throughput technology enabling the identification of specific transcription modulators,” SLAC Discov., 22, 378-386 (2017).

The ability of some compounds of the present invention to inhibit POLRMT were determined in a homogeneous TR-FRET Assay using high-throughput screening in a 384-well plate format. This method is used to monitor the activity of mitochondrial transcription through measurement of its product, a 407 bp long RNA transcript. Detection of the product is facilitated by hybridization of two DNA-oligonucleotide probes to specific and adjacent sequences within the RNA product sequence. Upon annealing of the probes, two fluorophores are coupled directly to an acceptor nucleotide probe (ATTO647, 5′), or introduced via a coupled streptavidin with a biotinylated donor nucleotide probe (Europium cryptate) that is brought into sufficient proximity to serve as a fluorescence-donor-acceptor pair. Thus, a FRET signal at 665 nm is generated upon excitation at 340 nm.

Proteins used as transcription factors (POLRMT: NP_005026.3, TFAM: NP_003192.1, TFB2M: NP_071761.1) are diluted from their stocks to working concentrations of 1 μM, 20 μM and 4 μM respectively, in a dilution buffer containing 20 mM Tris-HCl (pH 8.0), 200 mM NaCl, 10% (v/v) glycerol, 1 mM Dithiothreitol (DTT), 0.5 mM EDTA.

DNA template is a pUC18 plasmid with the mitochondrial light strand promotor sequence (1-477) cloned between HindIII and BamHI sites. The DNA template is restriction linearized proximal to the promotor 3′-end (pUC-LSP).

The reaction mixture (10 uL) containing 7.5 nM POLRMT, 15 nM of TFB2M, 30 nM of TFAM, 0.5 nM of DNA template and 500 μM nucleotide triphosphate mix (NTPs) in a reaction buffer (containing 10 mM Tris-HCl (pH 7.5), 10 mM MgCl₂, 40 mM NaCl, 10 mM DTT, 0.005% (w/v) Tween-20, 160 units/ml Rnase inhibitor and 0.1 mg/mL BSA) are dispensed to compounds in microplates, using a Thermo Multidrop® dispenser, and incubated at 37° C. in a VWR INCU-Line incubator for 60 minutes after mixing. No nucleotide triphosphate mix is added to negative control samples. Microplates with compounds to be tested in the assay are prepared from 10 mM compound stocks in 100% DMSO, equal amounts of DMSO without any compound are added to positive control and negative control samples.

During the incubation, a mix of the detection reagents is prepared in a buffer such that the enzymatic reaction is terminated due to chelating of Mg-ions and increased ionic strength, containing 50 mM Tris-HCl (pH 7.5), 700 mM NaCl, 20 mM EDTA, and 0.010% (w/v) Tween-20. Europium-streptavidin is pre-incubated with a 200-fold molar excess of a random sequence oligonucleotide to block unspecific binding of oligo, for two hours at ambient temperature in the dark. Afterwards, the blocked Europium-streptavidin is kept on ice until use.

At the end of the enzymatic reaction time, 5 μL detection oligo mix in the detection buffer is added, and assay plates are mixed and kept at ambient temperature for one hour, protected from light. The concentration of the Acceptor nucleotide oligo (e.g., ATTO647N-5′-ACAAAGAACCCTAACACCAG-3′) and Donor nucleotide oligo (e.g., bio-5′-AACACATCTCT(-bio)GCCAAACCCCA-bio-3′) in each assay well is 1 nM, and 3 nM, respectively.

After incubation with oligo mix, 5 μL of pre-blocked Europium-streptavidin reagent is dispensed to each assay well, assay plates are again mixed and kept at ambient temperature for one hour, protected from light.

The generated signal is measured with BMG Pherastar microtiter plate reader with a TRF light unit, using excitation at 340 nm, an integration time of 200 μs, and a delay time of 100 μs, before detection at 620 nm and 665 nm. The ratio of donor- and acceptor-fluorescence is used as a measure of the generated transcript product (i.e. enzymatic activity).

The IC₅₀ values are summarized in Table 1.

TABLE 1
IC₅₀ values for isoquinolinone compounds.

| Examples | IC₅₀ (μM) |
|---|---|
| Example 1 | 10 |
| Example 2 | >100 |
| Example 4 | 0.4 |
| Example 5 | 0.2 |
| Example 6 | 0.2 |
| Example 7 | 0.8 |
| Example 8 | 35 |
| Example 9 | 3 |
| Example 10 | >100 |
| Example 12 | 0.2 |
| Example 13 | 0.9 |
| Example 15 | 51 |
| Example 16 | 51 |
| Example 17 | 82 |
| Example 19 | >100 |
| Example 20 | 1 |
| Example 22 | 0.04 |
| Example 23 | 75 |
| Example 24 | 0.05 |
| Example 26 | 1 |
| Example 27 | 1 |
| Example 28 | 36 |
| Example 30 | 2 |
| Example 31 | 8 |
| Example 32 | 22 |
| Example 33 | >100 |
| Example 34 | 14 |
| Example 35 | 0.4 |
| Example 36 | 0.2 |
| Example 37 | 0.9 |
| Example 38 | >100 |
| Example 39 | >100 |
| Example 40 | 1 |
| Example 41 | 1 |
| Example 42 | 7 |
| Example 43 | >100 |
| Example 44 | 2 |
| Example 45 | >100 |
| Example 46 | 2 |
| Example 47 | 11 |
| Example 48 | 0.2 |
| Example 49 | 5 |
| Example 50 | 0.6 |
| Example 51 | 6 |
| Example 52 | >100 |
| Example 53 | 13 |
| Example 54 | >100 |
| Example 55 | 2 |
| Example 57 | 0.3 |
| Example 58 | 2 |
| Example 59 | 25 |
| Example 60 | 39 |
| Example 61 | 0.4 |
| Example 62 | 0.06 |
| Example 63 | 3 |
| Example 64 | 0.05 |
| Example 65 | 0.03 |
| Example 66 | 5 |
| Example 67 | 12 |
| Example 68 | 11 |
| Example 69 | 12 |
| Example 70 | 8 |
| Example 71 | 1 |
| Example 72 | 17 |
| Example 73 | 0.4 |
| Example 75 | 4 |
| Example 76 | 0.2 |
| Example 77 | 0.03 |
| Example 78 | 0.03 |
| Example 79 | 0.05 |
| Example 80 | 0.04 |
| Example 81 | 0.03 |
| Example 82 | 0.09 |
| Example 83 | 0.4 |
| Example 84 | 0.08 |
| Example 85 | 0.02 |
| Example 86 | 0.02 |
| Example 87 | 0.2 |
| Example 88 | 0.4 |
| Example 89 | 0.3 |
| Example 90 | 1 |
| Example 91 | 0.3 |
| Example 92 | 0.08 |
| Example 93 | 0.03 |
| Example 94 | 0.07 |
| Example 95 | 1 |
| Example 96 | 2 |
| Example 97 | 0.3 |
| Example 98 | 0.3 |
| Example 99 | 0.02 |
| Example 100 | 0.1 |
| Example 101 | 2 |
| Example 102 | 0.7 |
| Example 103 | 0.7 |
| Example 104 | 1 |
| Example 105 | 0.2 |
| Example 106 | 0.6 |
| Example 107 | 0.2 |
| Example 109 | 7 |
| Example 110 | 25 |
| Example 111 | 0.09 |
| Example 112 | 4 |
| Example 113 | 0.6 |
| Example 114 | 0.2 |
| Example 115 | 0.4 |
| Example 116 | 0.5 |
| Example 117 | 0.5 |
| Example 118 | 0.4 |
| Example 119 | 0.7 |
| Example 120 | 0.3 |
| Example 121 | 0.2 |
| Example 122 | 0.3 |
| Example 123 | 0.3 |
| Example 124 | 0.5 |
| Example 125 | 0.2 |
| Example 126 | 0.4 |
| Example 127 | 2 |
| Example 128 | 3 |
| Example 129 | 1 |
| Example 130 | 2 |
| Example 131 | 1 |
| Example 132 | 0.4 |
| Example 133 | 1 |
| Example 134 | 21 |
| Example 135 | 0.2 |
| Example 136 | 3 |
| Example 137 | 0.3 |
| Example 138 | 12 |
| Example 139 | 2 |

TABLE 2
IC₅₀ values for quinolinone compounds.

| Example | IC₅₀ (μM) |
|---|---|
| Example Q1 | >100 |
| Example Q2 | >100 |
| Example Q3 | 4 |
| Example Q4 | 23 |
| Example Q5 | 5 |
| Example Q6 | 18 |
| Example Q7 | 9 |
| Example Q8 | >100 |
| Example Q9 | >100 |
| Example Q10 | 2 |
| Example Q11 | 3 |
| Example Q12 | 18 |
| Example Q13 | 3 |
| Example Q14 | 7 |
| Example Q15 | 7 |

### General Protocol for In Vivo AML (Acute Myeloid Leukemia) Efficacy Experiment

### Determination of Maximum Tolerated Dose of Test Compound

Immunocompromised mice (6-10-week-old, female NSG mice, strain NOD.Cg-Prkdc^{scid} Il2rg^{tm1Wjl}/Szj, Jackson Laboratories) are treated orally with test compound ranging from 75 to 150 mg/kg, once or twice per day for the duration of 14 days. Total body weight is measured, and the general condition of mice is monitored routinely. Mice with severe symptoms and moribund are excluded from study. Submental blood collection method (no anesthesia) is used for all samplings. Plasma levels of test compound are determined at intervals ranging from 0.5 to 4 hours post first and last doses in all dosing groups. From these data pharmacokinetic analysis are conducted.

### In Vivo Efficacy Study in AML Mouse Model

MV4-11 AML cell lines (ATCC) are labelled with luciferase tag by viral transduction procedure (MV4-11-luc).

For an AML cell line xenograft efficacy experiment, female NSG mice are given intravenously ˜1×10⁷ MV4-11-luc cells. Mice are flux sorted and randomized into treatment groups 14 days post transplantation. Mice are then treated with vehicle (50 mM Na₂HPO₄), or test compound at a tolerable dose determined from the above study, once or twice per day for 21 days. Tumor progression/regression is monitored by imaging of mice using luciferin as a substrate (150 mg/kg). Images are taken on a total of 9 time points i.e., one flux sort and once weekly to end date (8 time points). Imaging is performed under anesthesia and using in vivo imaging equipment IVIS. The treatment efficacy is also measured based on proportion of human AML cells, determined by flow cytometry analysis of viable human CD45 positive cell population in peripheral blood of mice one week post last dose. Plasma levels of test compound are determined at intervals ranging from 0.5 to 4 hours post last dose. Animals are monitored individually, and total body weight is measured routinely. The endpoint of the experiment is moribundity. In addition, mice demonstrating tumor-associated symptoms including impairment of hind limb function, ocular proptosis, and weight loss are considered for euthanasia. The remaining mice are euthanized on day 60 of the study.

1. A compound of formula (I), or a pharmaceutically acceptable salt thereof: 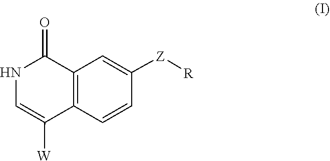
wherein:
Z is O, C(H)(R¹), C₆H₄, C(O), C(O)N(R⁷), or N(R²);
W is C₃-C₈ cycloalkyl, C₄-C₁₂ bicyclic, C₄-C₁₀ cycloalkenyl, C₆-C₁₂ aryl, or 5- to 12-membered heteroaryl, any of which is substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl;
R is hydrogen, or C₁-C₆ alkyl optionally substituted with one or more groups each independently selected from the group consisting of fluoro, hydroxyl, C₁-C₄ alkoxyl, cyano, C₁-C₄ haloalkoxyl, carboxyl, C(O)NR⁵R⁶, NR⁵R⁶ and NR²R³,
or R is C₆-C₁₂ aryl optionally substituted with one or more groups, each independently selected from the group consisting of fluoro, chloro, C₁-C₄ alkyl, trifluoromethyl, difluoromethyl, cyano, hydroxyl, NR⁵R⁶, and C₁-C₄ alkoxyl,
or R is hydroxyl, NR¹R², C(O)R³, C₁-C₄ haloalkoxyl, CH₂CH₂R⁸, CR³R⁴C(O)OR⁵, or 4-7 membered cyclic ring containing one or more heteroatoms, such cyclic ring is optionally substituted with C(O)R¹, NR¹—C(O)—R¹, or CR³R⁴C(O)NR⁵R⁶;
each R¹ is independently hydrogen, C₁-C₄ alkoxyl, or C₁-C₄ alkyl optionally substituted with one or more fluoro groups;
each R² is independently hydrogen, C₁-C₄ alkyl optionally substituted with one or more fluoro groups, C(O)C₁-C₄ alkyl optionally substituted with one or more fluoro or methoxyl groups, C(O)-cycloheteroalkyl optionally substituted with methyl or acetate, or C(O)NHC₁-C₄ alkyl optionally substituted with one or more fluoro groups;
R³ and R⁴ are each independently hydrogen or C₁-C₄ alkyl optionally substituted with one or more groups each independently selected from the group consisting of fluoro, hydroxyl, C₁-C₄ alkoxyl, C₁-C₄ haloalkoxyl, and NR⁵R⁶;
R⁵ and R⁶ are each independently hydrogen, cycloheteroalkyl optionally substituted with acetate, or C₁-C₄ alkyl optionally substituted with one or more groups each independently selected from the group consisting of fluoro, hydroxyl, cyano, C₁-C₄ alkoxyl, C₁-C₄ haloalkoxyl, and NR¹R²,
or if R⁵ and R⁶ are attached to the same nitrogen atom, R⁵ and R⁶ together with their connecting nitrogen form a 3- to 7-membered heterocyclic ring or 6- to 12-membered heterobicyclic ring, each such heterocyclic ring or heterobicyclic ring optionally containing another heteroatom that is N, O, S, S(O), SO₂R¹, or S(O)(NR¹) and each such heterocyclic ring or heterobicyclic ring is optionally substituted with one or more groups each independently selected from the group consisting of fluoro, chloro, and C₁-C₄ alkyl optionally substituted with one or more fluoro, C₁-alkoxyl or hydroxyl, and each such heterocyclic ring or heterobicyclic ring is further optionally substituted with cycloalkyl, cyano, carboxyl, C(O)R¹, C(O)NR¹R², imine, oxo, NR¹—C(O)—R¹, SO₂R¹, or C₁-C₄ alkylcarboxylate;
R⁷ is H or C₁-C₆ alkyl; and
R⁸ is aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl,
or R⁸ is OR¹, NR¹R², C(O)R¹, or C(O)NR²R².

2. The compound according to claim 1, or a pharmaceutically acceptable salt thereof, wherein:
Z is O, C(H)(R¹), C₆H₄, C(O), C(O)N(R⁷), or N(R²);
W is C₆ cycloalkyl, C₅ bicyclic, C₆ cycloalkenyl, C₆ aryl or 5-membered heteroaryl, any of which is substituted with one or more groups, each independently selected from the group consisting of fluoro, chloro, cyano, and C₁-C₂ alkyl optionally substituted with one or more deuterium or hydroxyl;
R is hydrogen, or C₁-C₄ alkyl optionally substituted with one or more groups each independently selected from the group consisting of fluoro, hydroxyl, C₁ alkoxyl, cyano, carboxyl, C(O)NR⁵R⁶, and NR²R³,
or R is hydroxyl, NR¹R², C(O)R³, CR³R⁴C(O)OR⁵, or 4-7 membered cyclic ring containing one or more heteroatoms, such cyclic ring is optionally substituted with C(O)R¹, NR¹—C(O)—R¹, or CR³R⁴C(O)NR⁵R⁶;
each R¹ is independently hydrogen, C₁ alkoxyl, or C₁-C₂ alkyl;
each R² is independently hydrogen, C₁-C₂ alkyl optionally substituted with one or more fluoro groups, C(O)C₁-C₄ alkyl optionally substituted with methoxy, or C(O)-cycloheteroalkyl optionally substituted with methyl or acetate;
R³ and R⁴ are each independently hydrogen or C₁ alkyl optionally substituted with C₁ alkoxyl;
R⁵ and R⁶ are each independently hydrogen, C₁-C₂ alkyl, or cycloheteroalkyl substituted with acetate;
or if R⁵ and R⁶ are attached to the same nitrogen atom, R⁵ and R⁶ together with their connecting nitrogen form a 5- or 6-membered heterocyclic ring or 8-membered heterobicyclic ring, each such heterocyclic ring or heterobicyclic ring optionally containing another heteroatom that is N, O, S, S(O), SO₂R¹, or S(O)(NR¹) and each such heterocyclic ring or heterobicyclic ring is optionally substituted with one or more groups each independently selected from the group consisting of fluoro and C₁-C₂ alkyl optionally substituted with one or more fluoro, C₁-alkoxyl or hydroxyl, and each such heterocyclic ring or heterobicyclic ring is further optionally substituted with cyano, carboxyl, C(O)R¹, C(O)NR¹R², NR¹—C(O)—R¹, or SO₂R¹; and
R⁷ is C₁ alkyl.

3. The compound according to claim 2, or a pharmaceutically acceptable salt thereof, wherein:
Z is O or C₆H₄;
W is C₆ aryl substituted with one or more groups, each independently selected from the group consisting of fluoro, chloro, and C₁-C₂ alkyl;
R is hydrogen, C₁-C₄ alkyl optionally substituted with one or more groups each independently selected from the group consisting of fluoro, C₁ alkoxyl, cyano, carboxyl, and C(O)NR⁵R⁶;
each R¹ is independently hydrogen or C₁-C₂ alkyl;
each R² is independently hydrogen;
R⁵ and R⁶ are each independently hydrogen;
or if R⁵ and R⁶ are attached to the same nitrogen atom, R⁵ and R⁶ together with their connecting nitrogen form a 5- or 6-membered heterocyclic ring or 8-membered heterobicyclic ring, each such heterocyclic ring or heterobicyclic ring optionally containing another heteroatom that is N or O, and each such heterocyclic ring or heterobicyclic ring is optionally substituted with one or more groups each independently selected from the group consisting of fluoro and C₁-C₂ alkyl optionally substituted with one or more fluoro, or hydroxyl, and each such heterocyclic ring or heterobicyclic ring is further optionally substituted with cyano, carboxyl, C(O)R¹, C(O)NR¹R², or SO₂R¹.

4. The compound according to claim 3, or a pharmaceutically acceptable salt thereof, wherein:
Z is O;
W is C₆ aryl substituted with one or more groups, each independently selected from the group consisting of fluoro, chloro, and C₁ alkyl;
R is C₂ alkyl substituted with C(O)NR⁵R⁶; and
R⁵ and R⁶ are attached to the same nitrogen atom, and together with their connecting nitrogen form a 6-membered heterocyclic ring substituted with carboxyl.

5. The compound of claim 3, or a pharmaceutically acceptable salt thereof, wherein:
Z is O;
W is C₆ aryl substituted with one or more groups, each independently selected from the group consisting of fluoro and C₁ alkyl; and
R is C₁ alkyl substituted with cyano.

6. The compound of claim 3, or a pharmaceutically acceptable salt thereof, wherein:
Z is O;
W is C₆ aryl substituted with two C₁ alkyl; and
R is C₁ alkyl substituted with cyano.

7. A pharmaceutical composition comprising the compound of claim 2, and a pharmaceutically acceptable excipient.

8. A pharmaceutical composition comprising the compound of claim 3, and a pharmaceutically acceptable excipient.

9. A compound, or a pharmaceutically acceptable salt thereof, selected from the group consisting of:

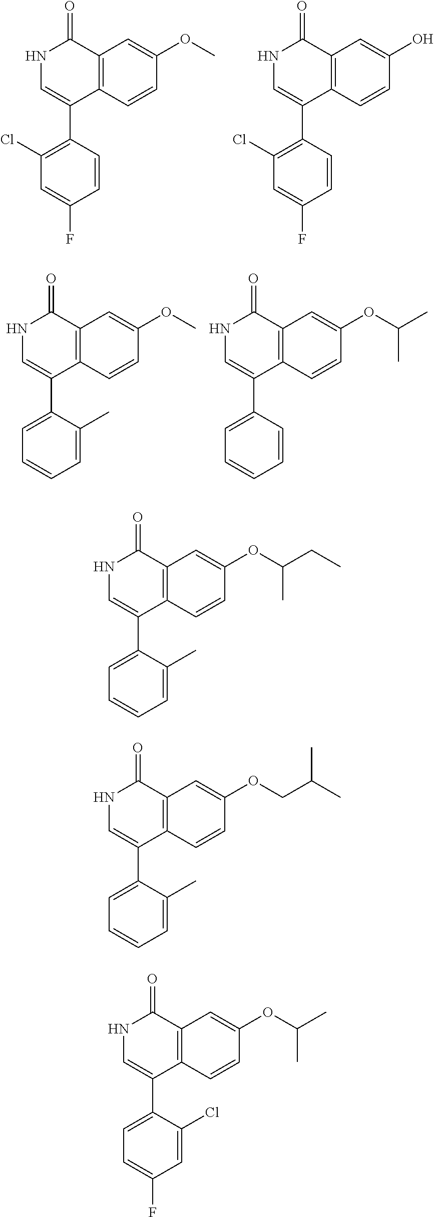

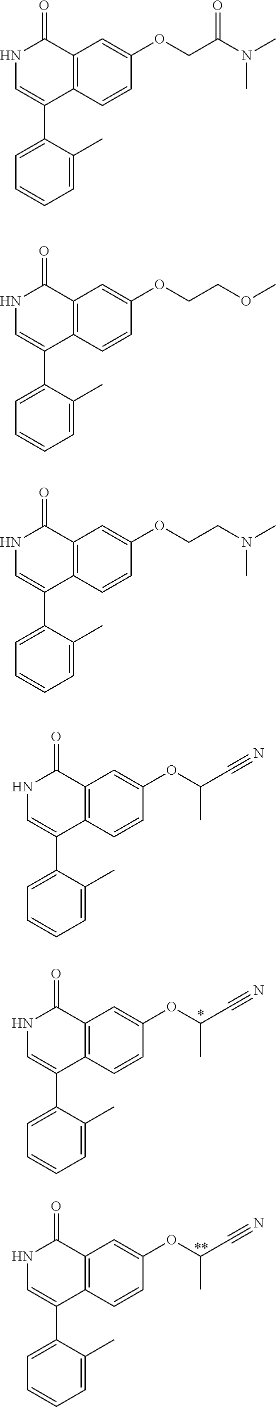

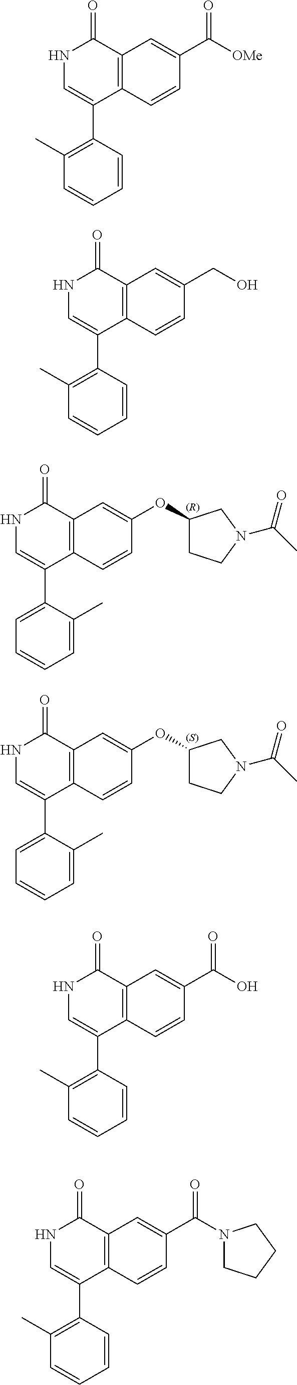

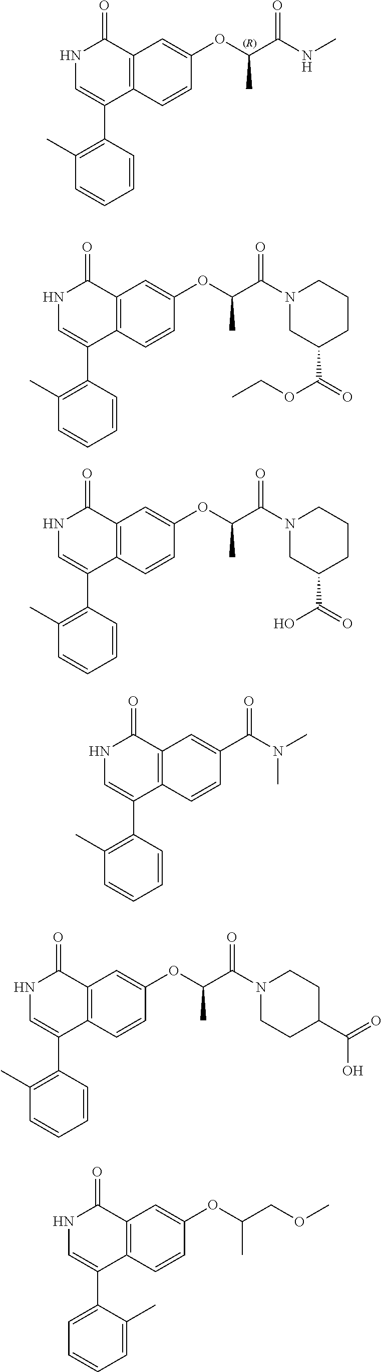

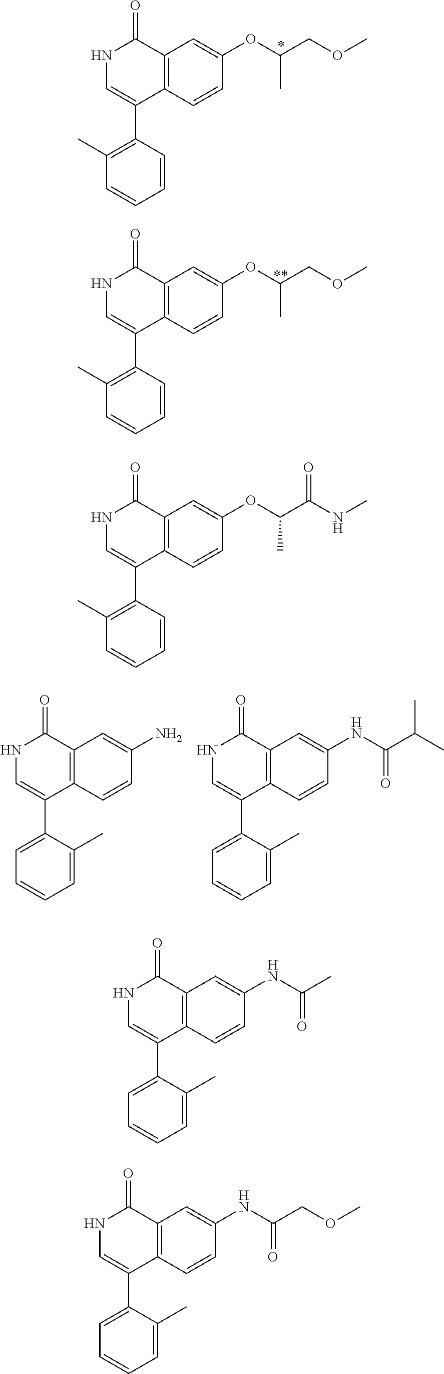

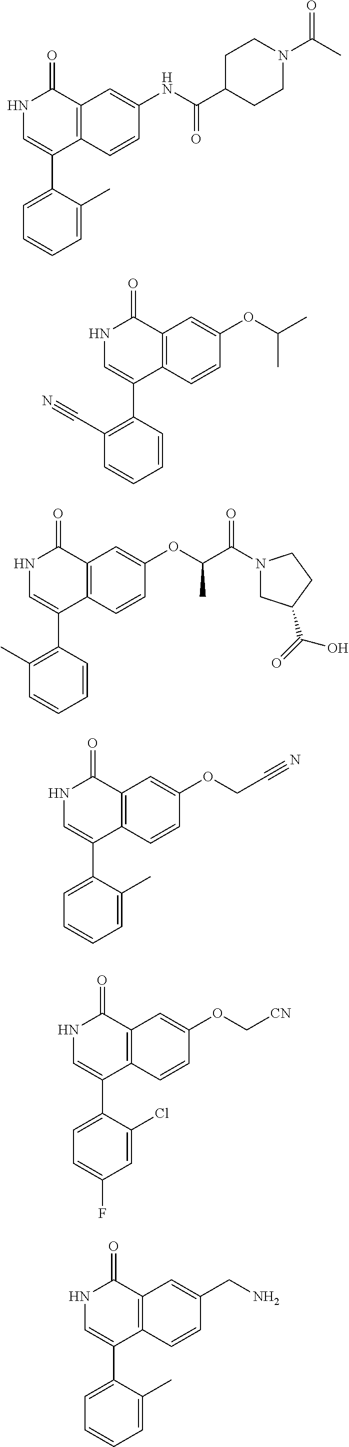

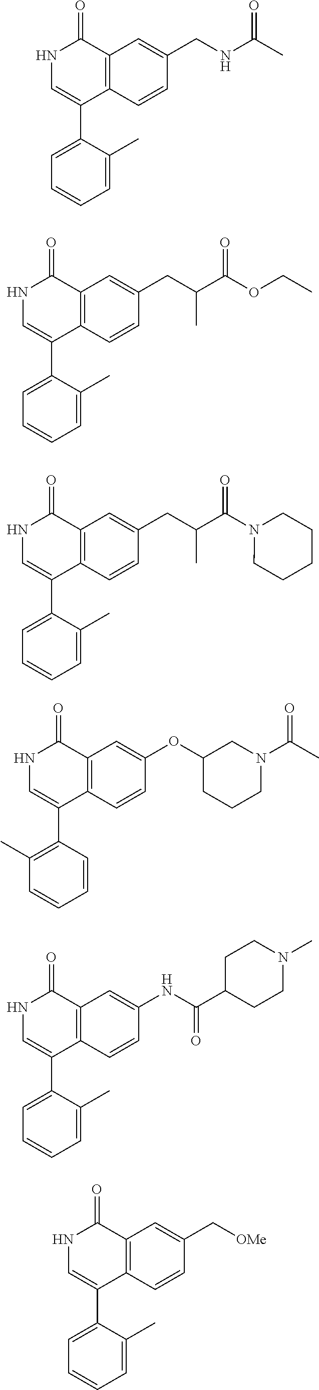

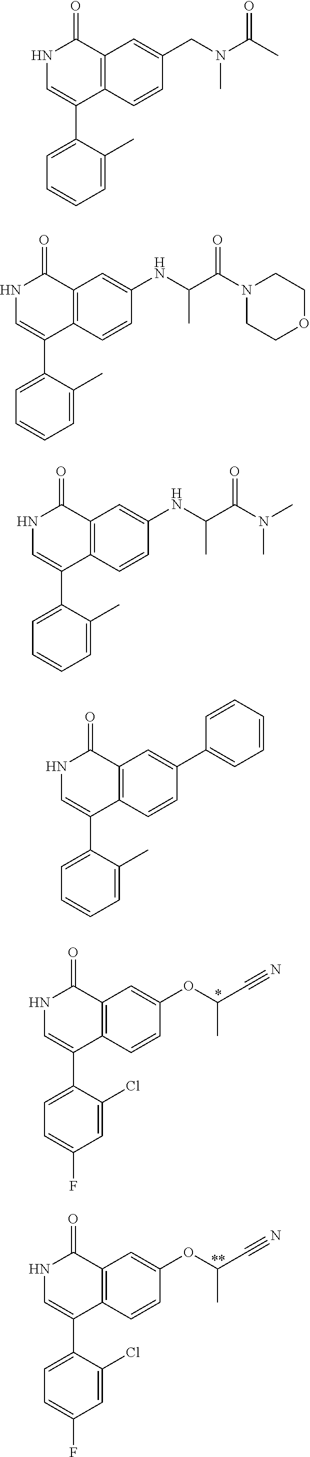

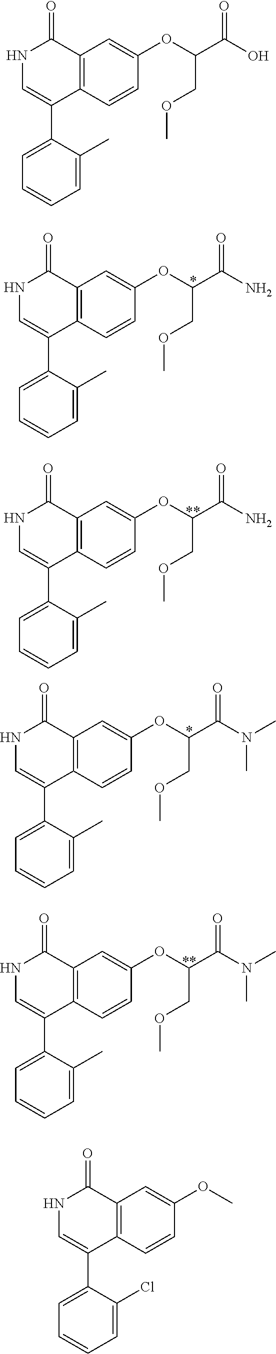

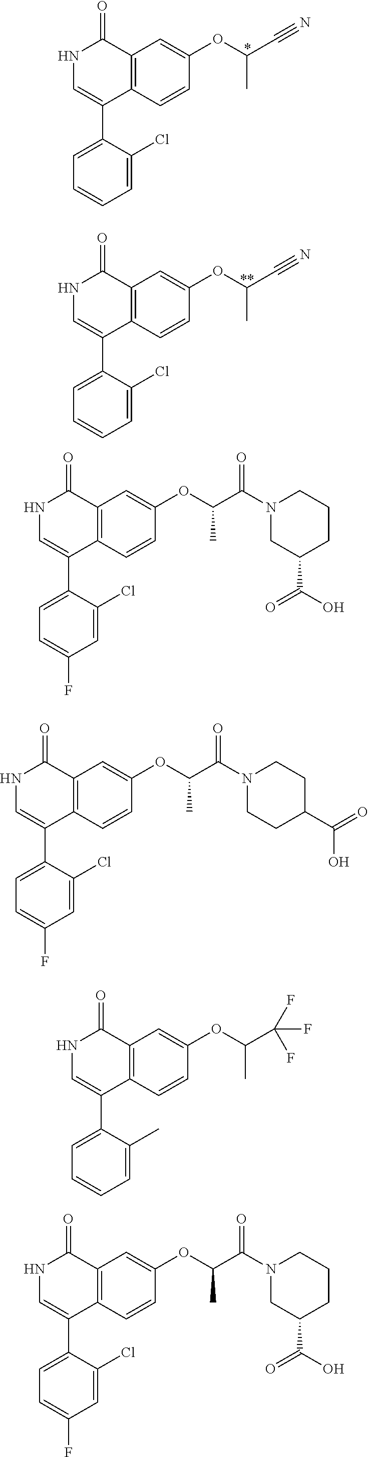

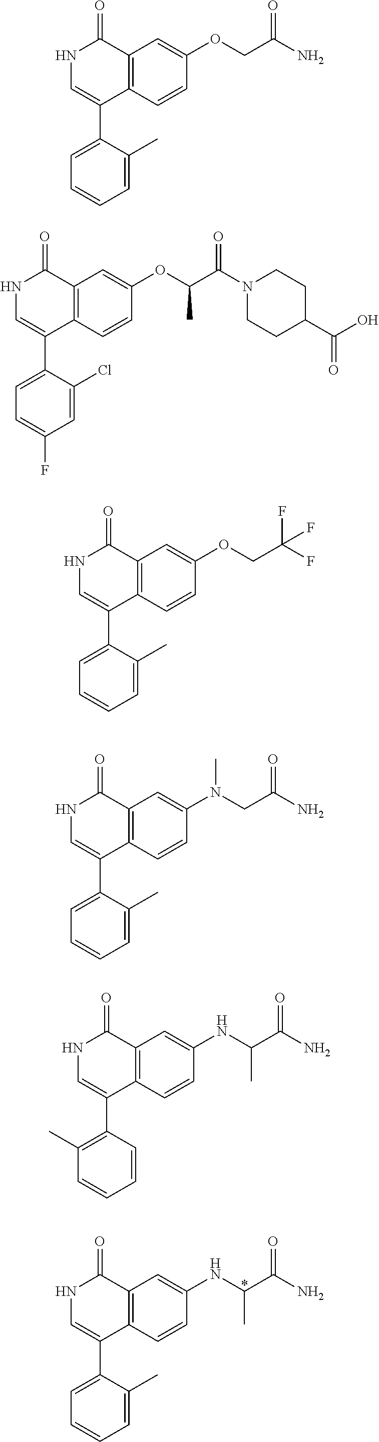

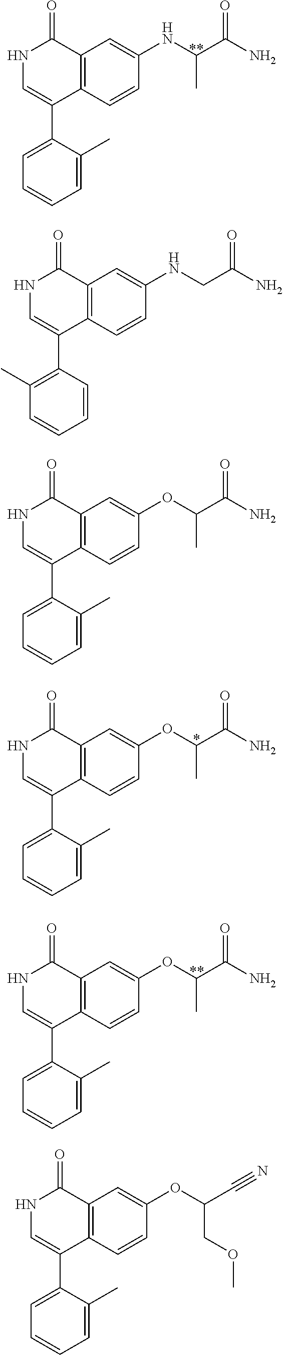

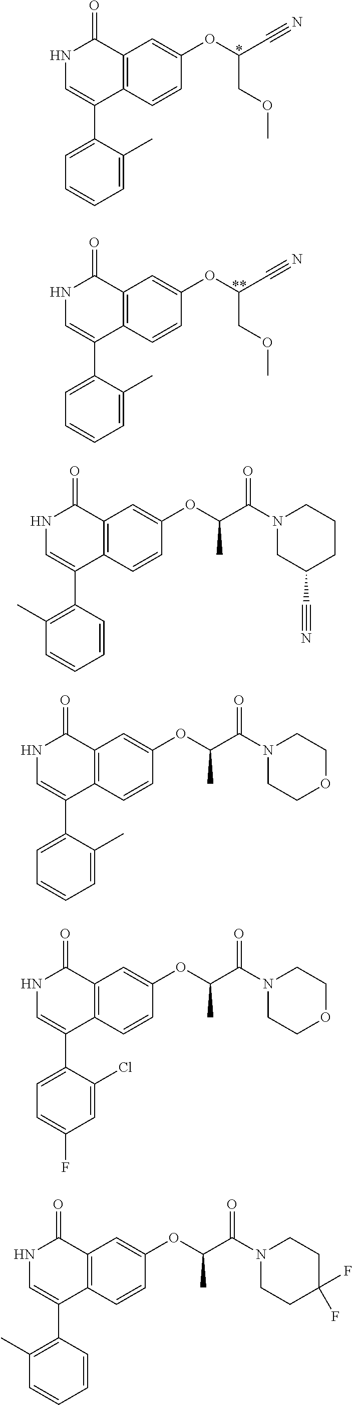

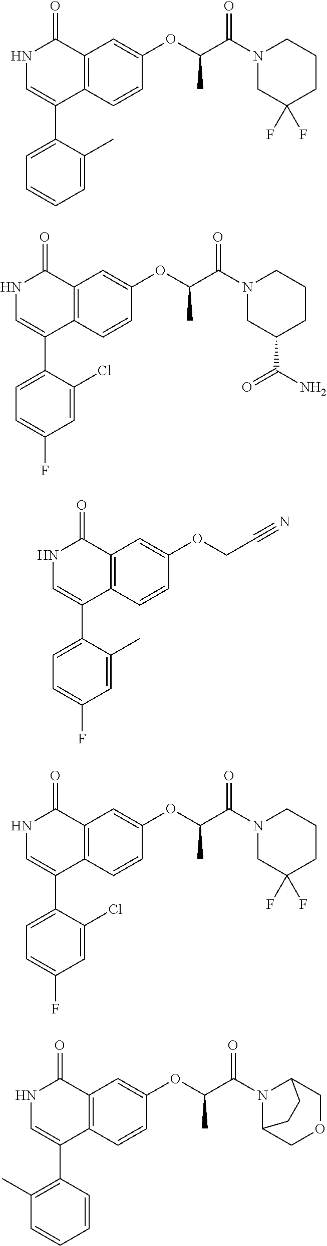

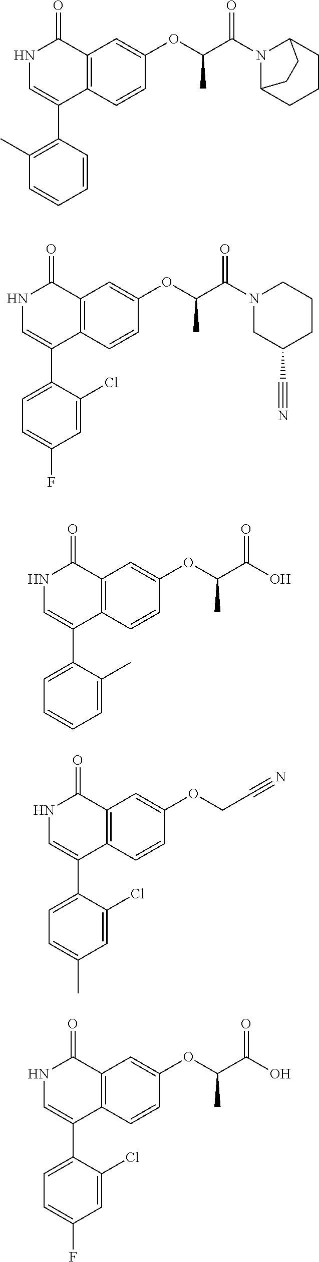

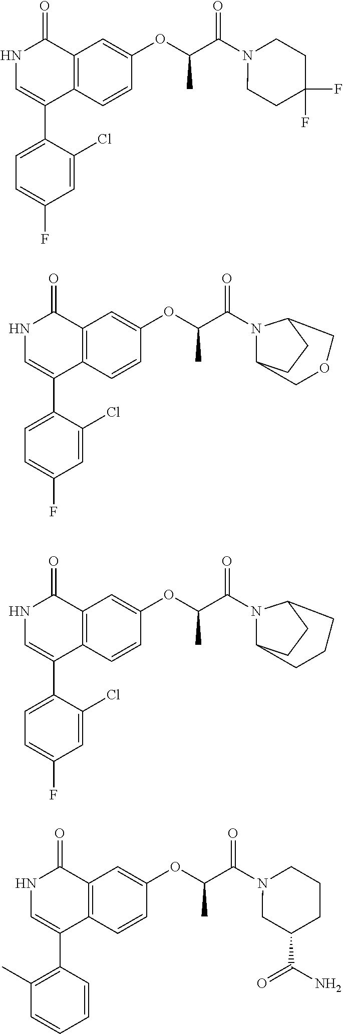

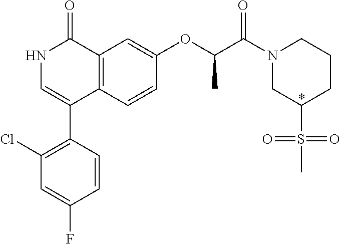 [SMILES: C[C@@H](Oc1ccc2c(-c3ccc(F)cc3Cl)c[nH]c(=O)c2c1)C(=O)N1CCCC(S(C)(=O)=O)C1]

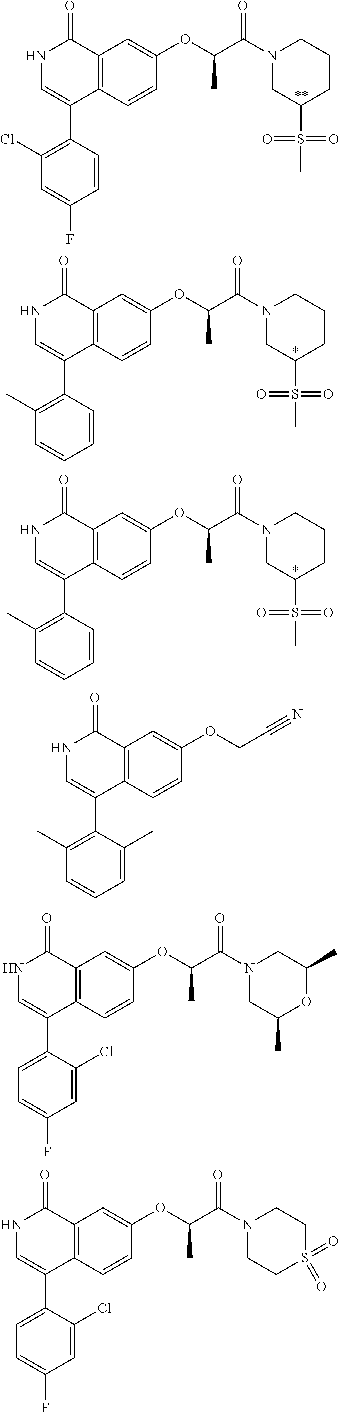

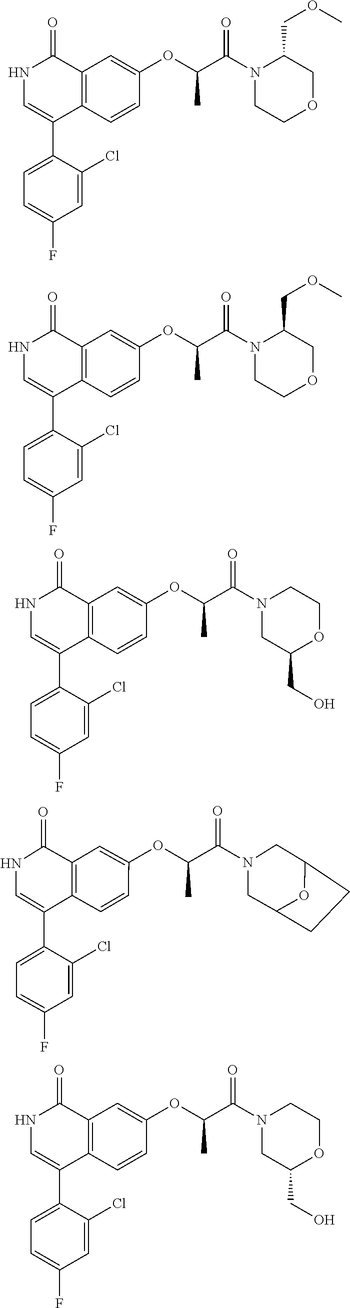

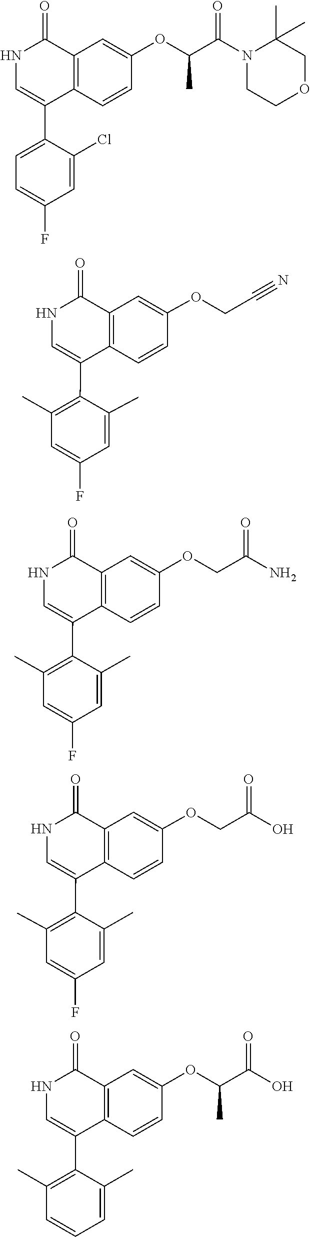

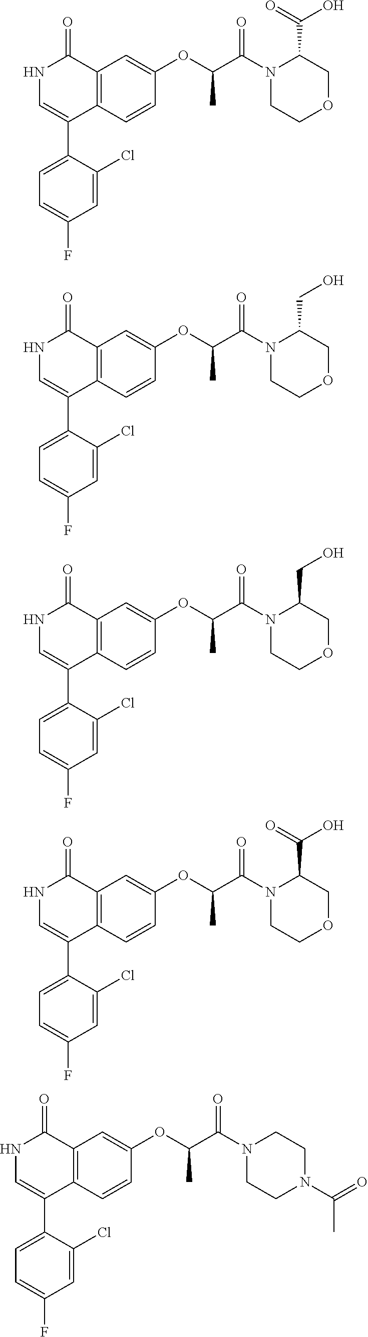

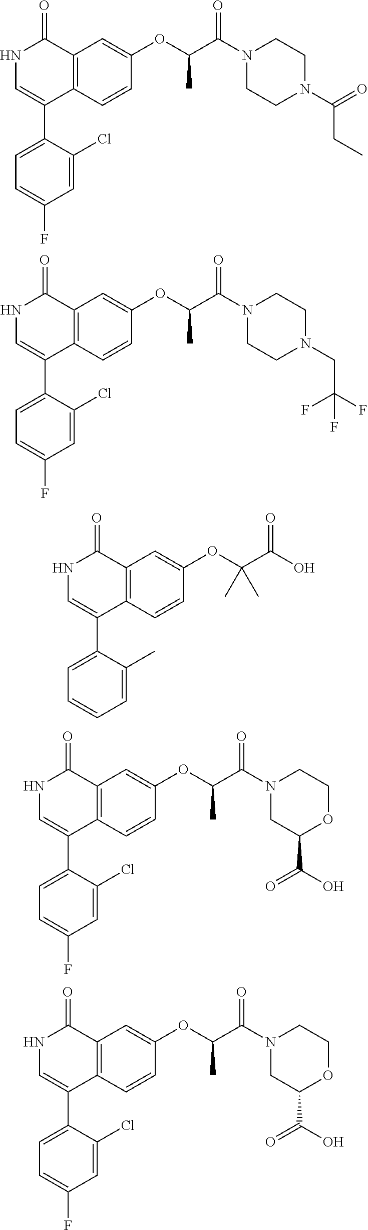

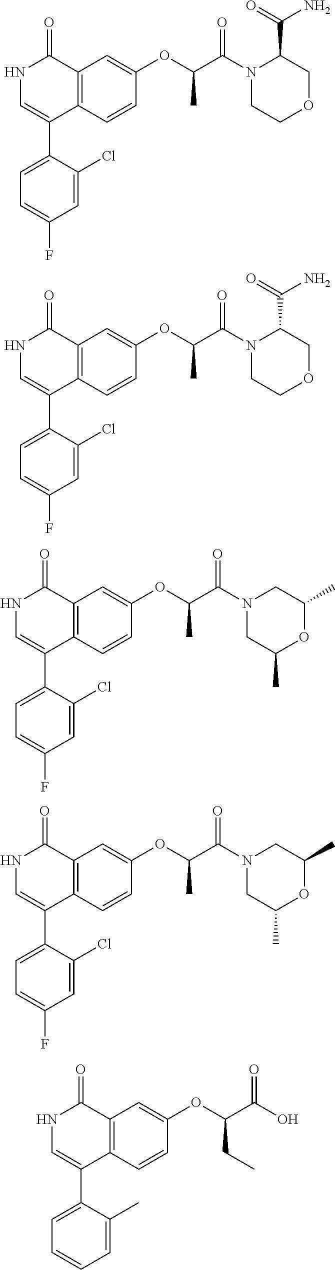

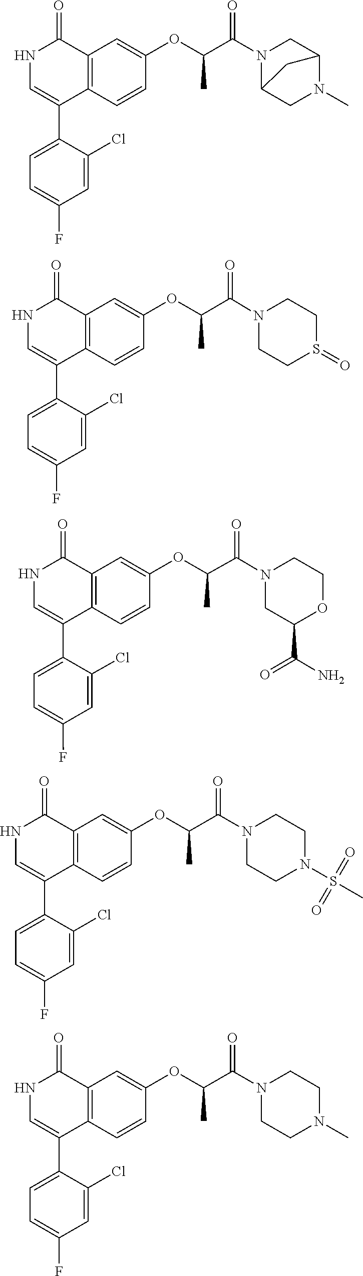

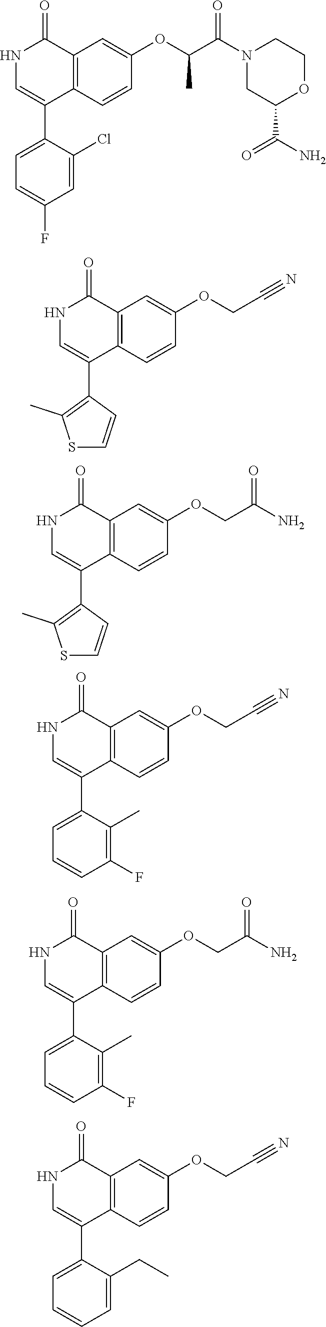

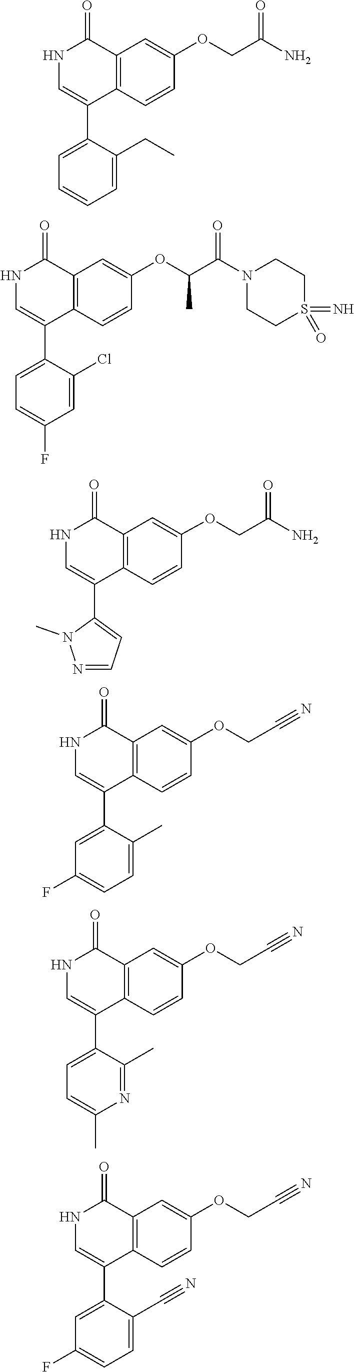

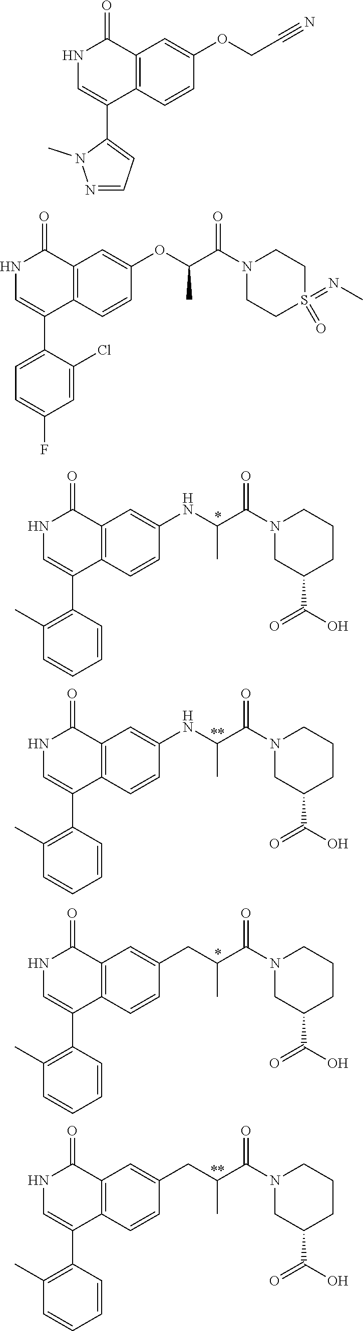

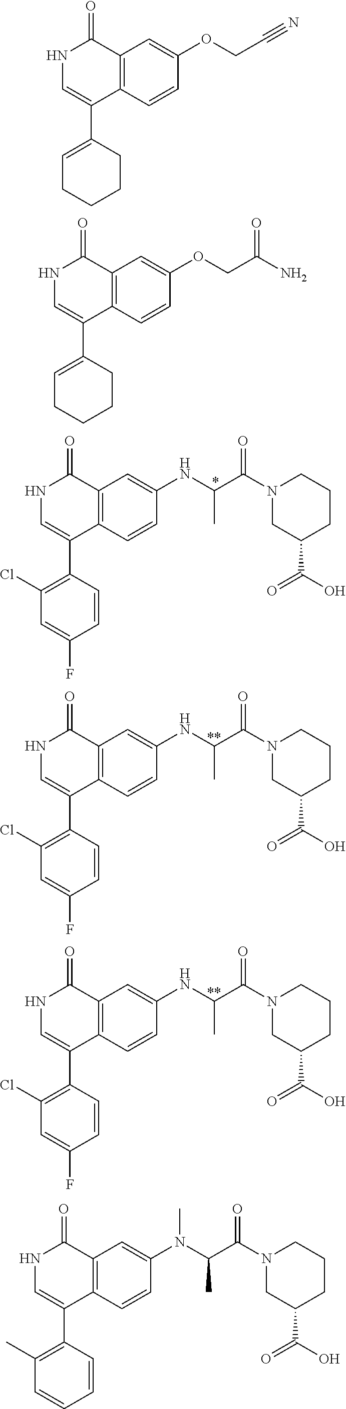

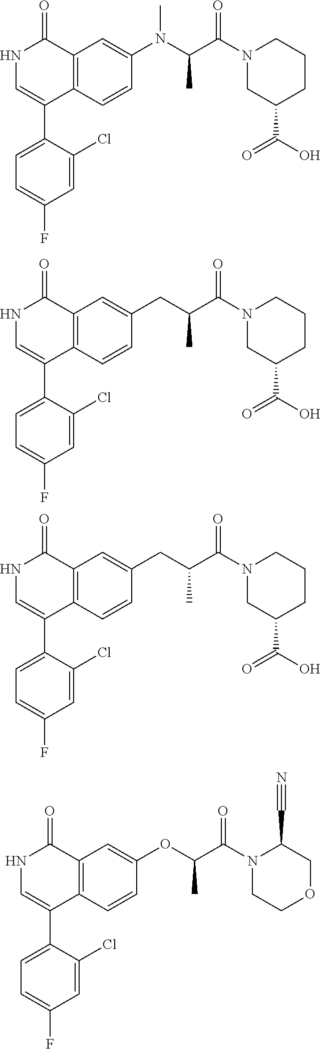

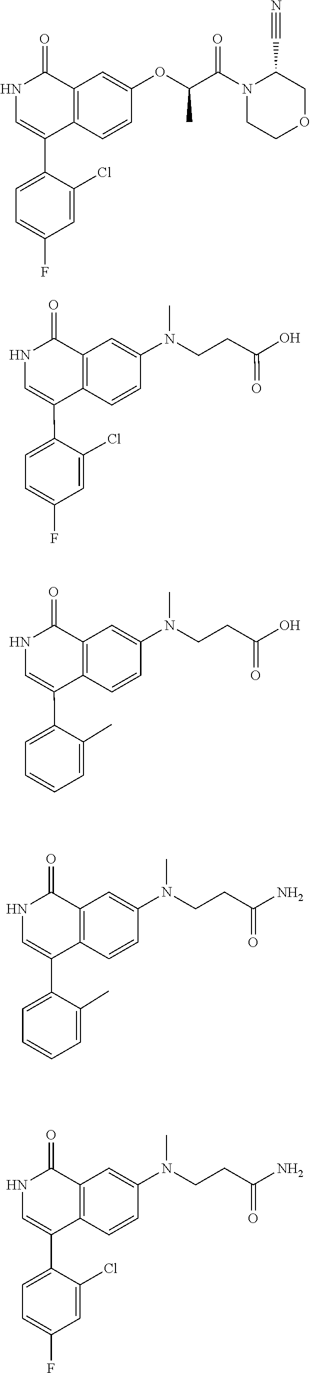

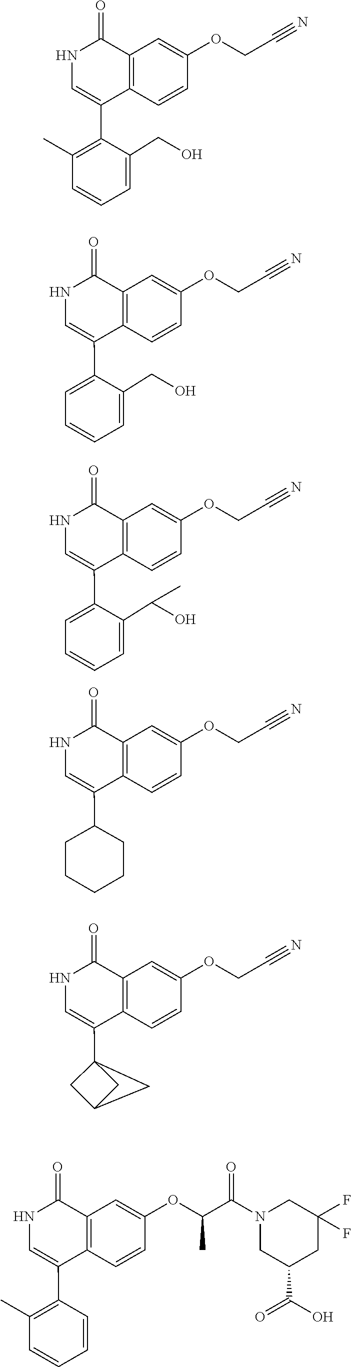

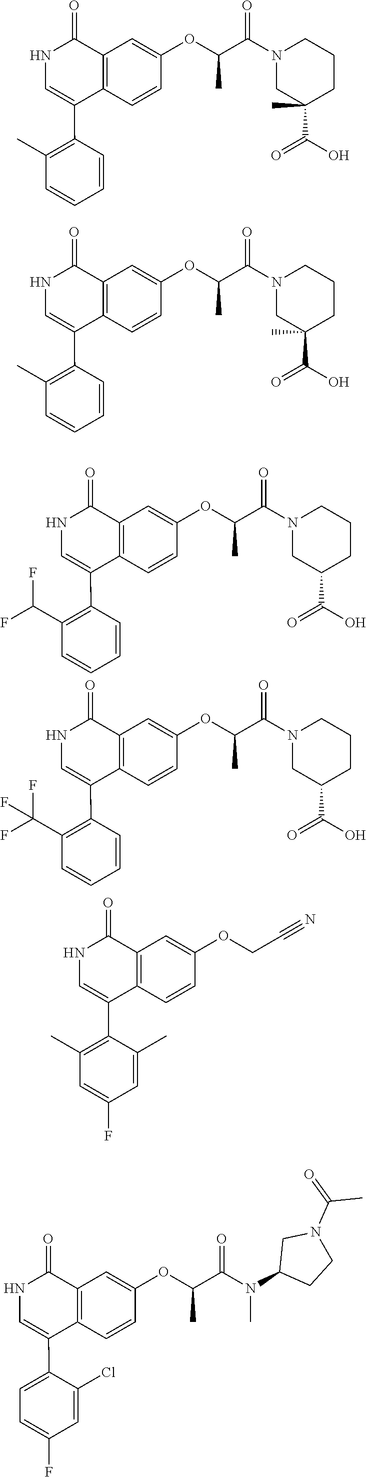

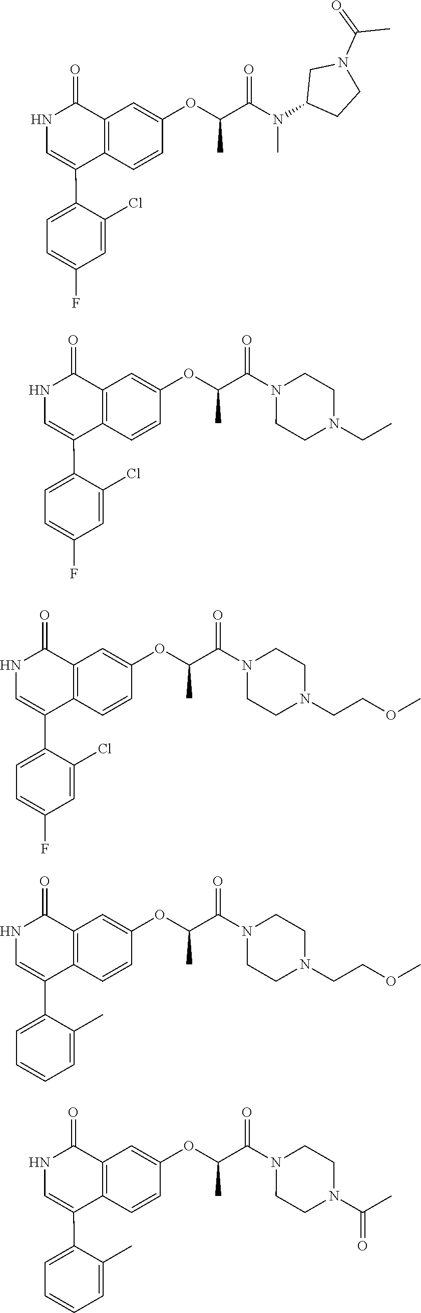

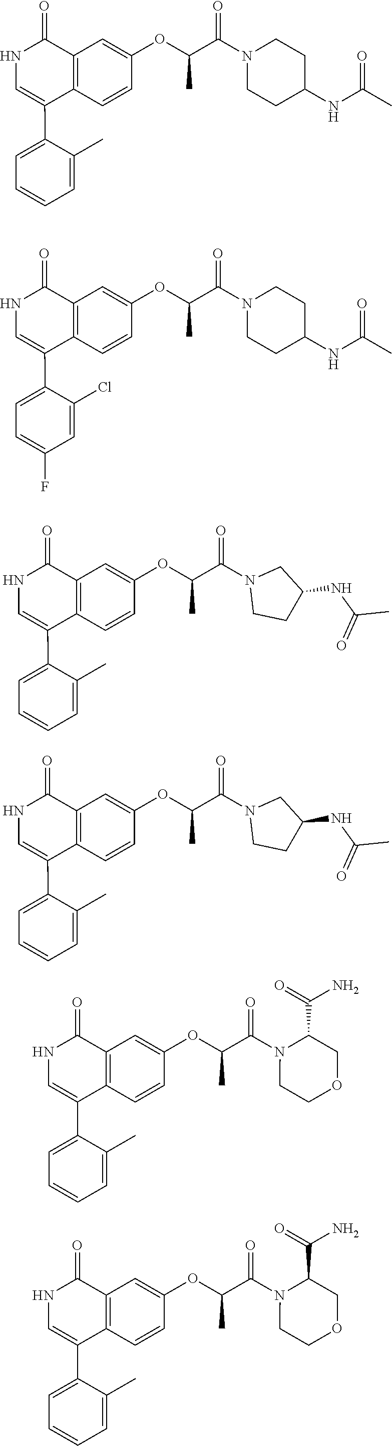

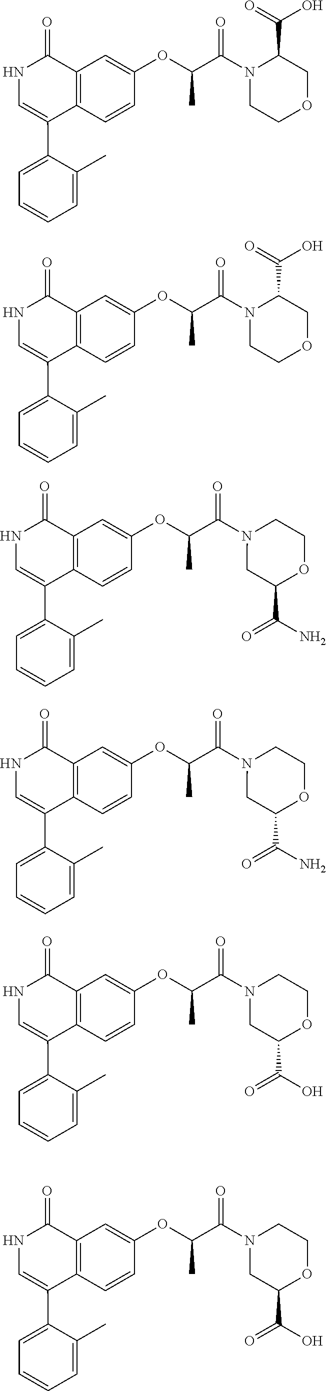

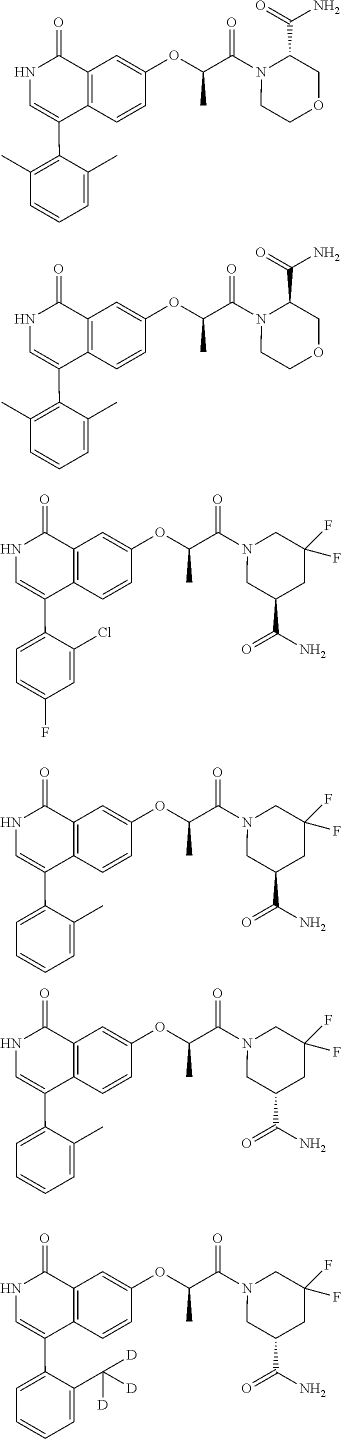

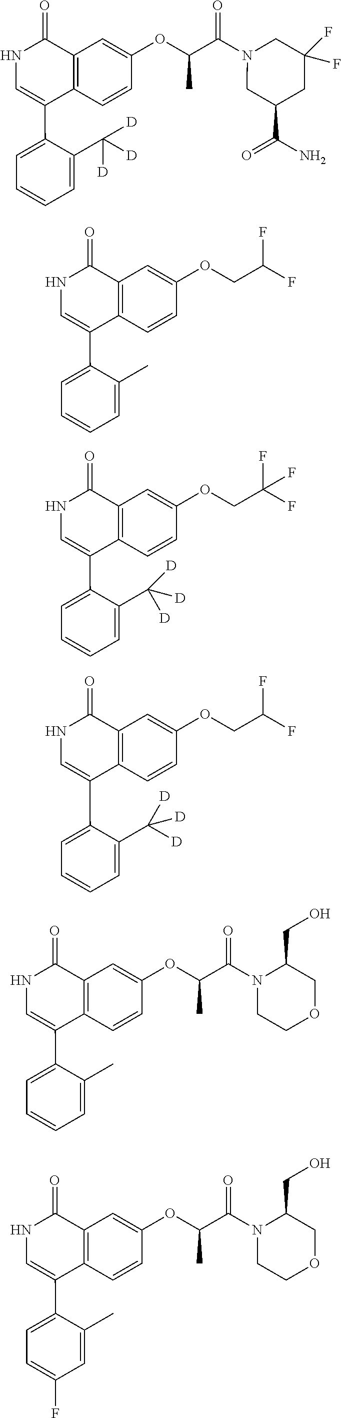

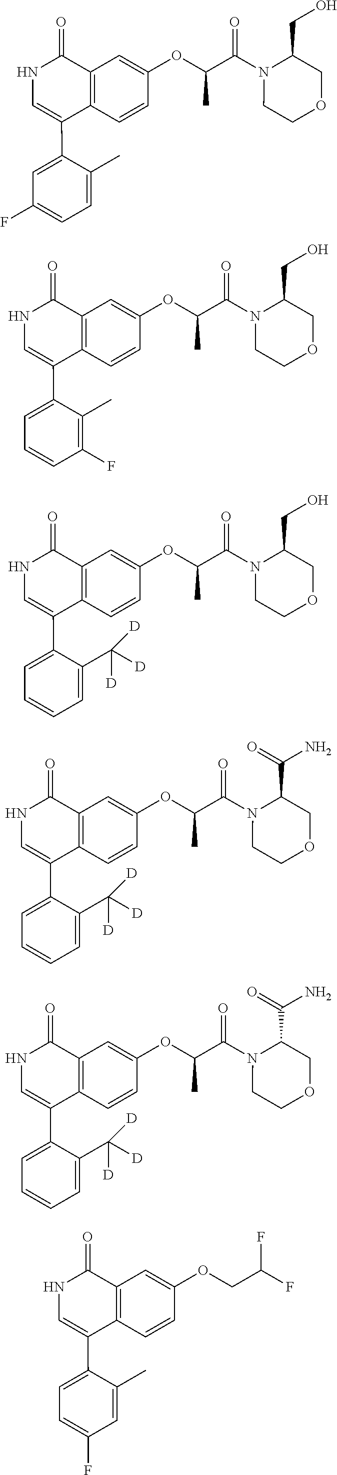

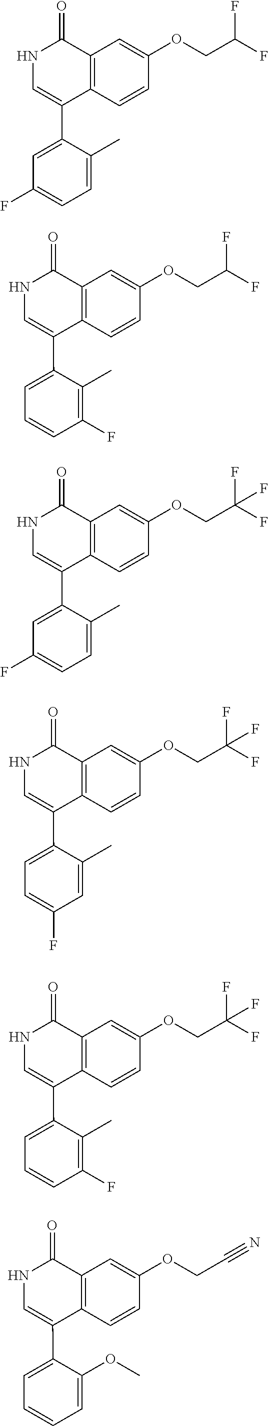

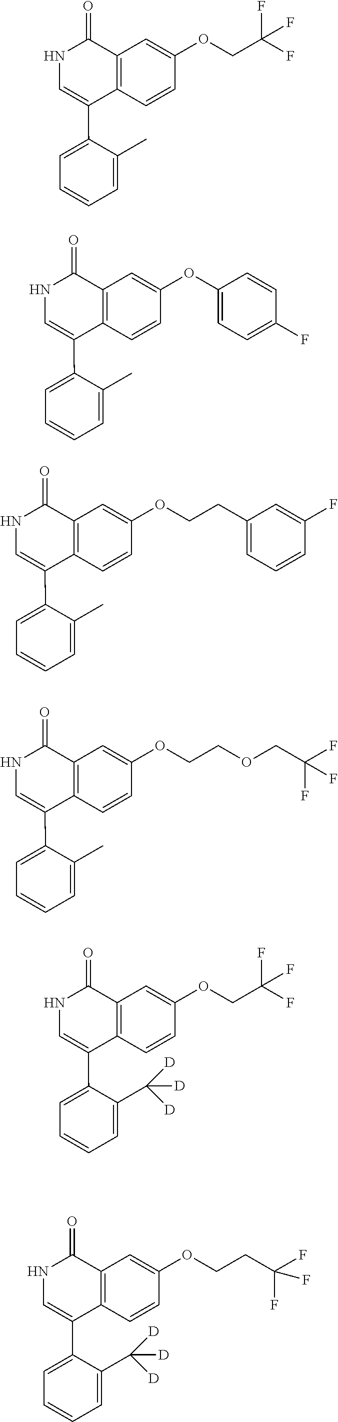

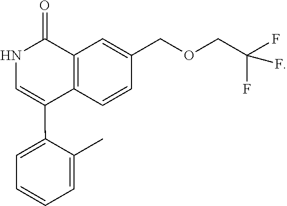 [SMILES: Cc1ccccc1-c1c[nH]c(=O)c2cc(COCC(F)(F)F)ccc12]

10. The compound of claim 9, or a pharmaceutically acceptable salt thereof, wherein the compound is selected from the group consisting of:

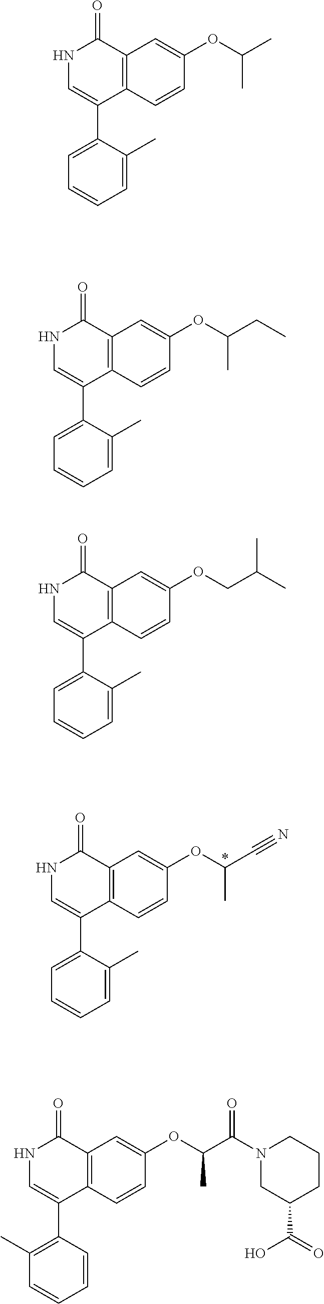

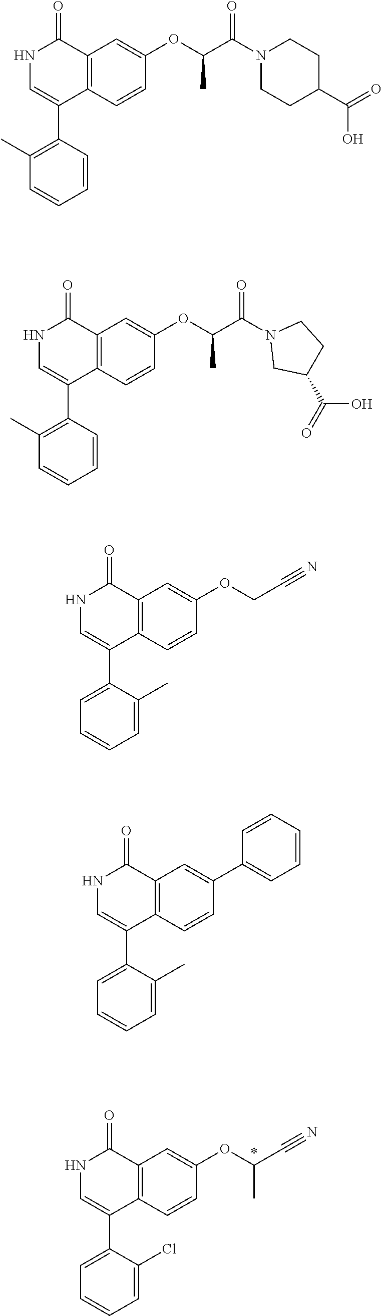 [SMILES: Cc1ccccc1-c1c[nH]c(=O)c2cc(O[C@H](C)C(=O)N3CCC(C(=O)O)CC3)ccc12 ; Cc1ccccc1-c1c[nH]c(=O)c2cc(O[C@H](C)C(=O)N3CC[C@H](C(=O)O)C3)ccc12 ; Cc1ccccc1-c1c[nH]c(=O)c2cc(OCC#N)ccc12 ; Cc1ccccc1-c1c[nH]c(=O)c2cc(-c3ccccc3)ccc12 ; CC(C#N)Oc1ccc2c(-c3ccccc3Cl)c[nH]c(=O)c2c1]

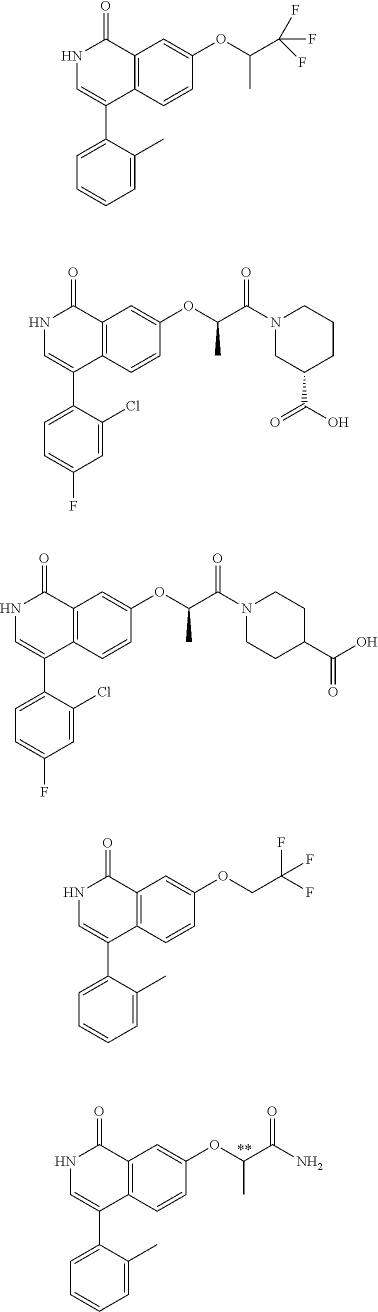 [SMILES: Cc1ccccc1-c1c[nH]c(=O)c2cc(OC(C)C(F)(F)F)ccc12 ; C[C@@H](Oc1ccc2c(-c3ccc(F)cc3Cl)c[nH]c(=O)c2c1)C(=O)N1CCC[C@H](C(=O)O)C1 ; C[C@@H](Oc1ccc2c(-c3ccc(F)cc3Cl)c[nH]c(=O)c2c1)C(=O)N1CCC(C(=O)O)CC1 ; Cc1ccccc1-c1c[nH]c(=O)c2cc(OCC(F)(F)F)ccc12 ; Cc1ccccc1-c1c[nH]c(=O)c2cc(OC(C)C(N)=O)ccc12]

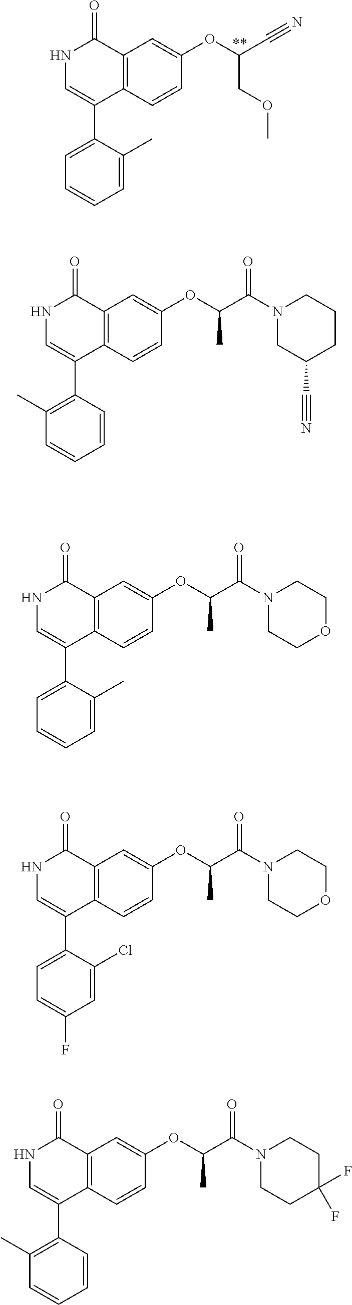 [SMILES: COCC(C#N)Oc1ccc2c(-c3ccccc3C)c[nH]c(=O)c2c1 ; Cc1ccccc1-c1c[nH]c(=O)c2cc(O[C@H](C)C(=O)N3CCC[C@H](C#N)C3)ccc12 ; Cc1ccccc1-c1c[nH]c(=O)c2cc(O[C@H](C)C(=O)N3CCOCC3)ccc12 ; C[C@@H](Oc1ccc2c(-c3ccc(F)cc3Cl)c[nH]c(=O)c2c1)C(=O)N1CCOCC1 ; Cc1ccccc1-c1c[nH]c(=O)c2cc(O[C@H](C)C(=O)N3CCC(F)(F)CC3)ccc12]

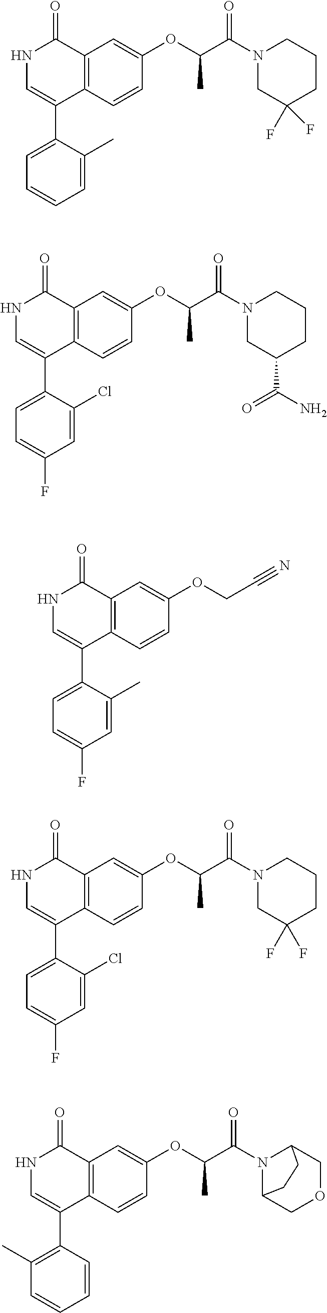 [SMILES: Cc1ccccc1-c1c[nH]c(=O)c2cc(O[C@H](C)C(=O)N3CCCC(F)(F)C3)ccc12 ; C[C@@H](Oc1ccc2c(-c3ccc(F)cc3Cl)c[nH]c(=O)c2c1)C(=O)N1CCC[C@H](C(N)=O)C1 ; Cc1cc(F)ccc1-c1c[nH]c(=O)c2cc(OCC#N)ccc12 ; C[C@@H](Oc1ccc2c(-c3ccc(F)cc3Cl)c[nH]c(=O)c2c1)C(=O)N1CCCC(F)(F)C1 ; Cc1ccccc1-c1c[nH]c(=O)c2cc(O[C@H](C)C(=O)N3C4CCC3COC4)ccc12]

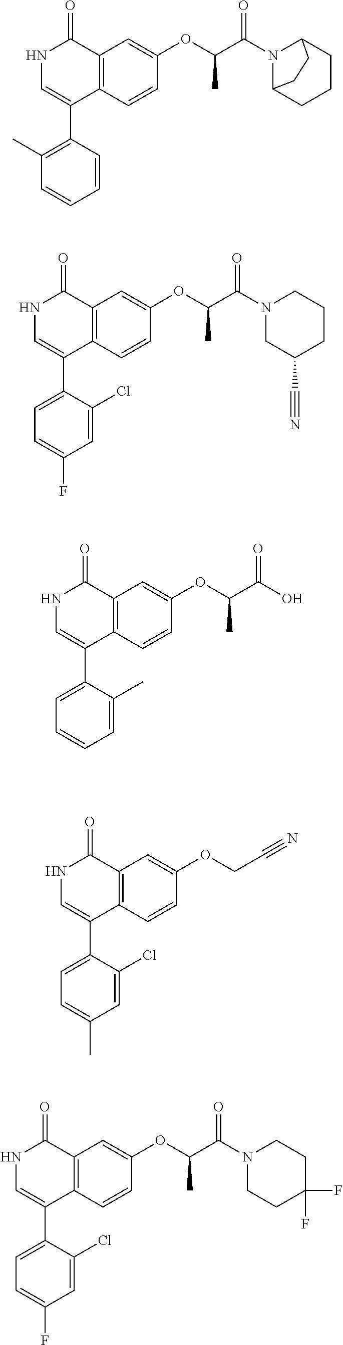 [SMILES: Cc1ccccc1-c1c[nH]c(=O)c2cc(O[C@H](C)C(=O)N3C4CCCC3CC4)ccc12 ; C[C@@H](Oc1ccc2c(-c3ccc(F)cc3Cl)c[nH]c(=O)c2c1)C(=O)N1CCC[C@H](C#N)C1 ; Cc1ccccc1-c1c[nH]c(=O)c2cc(O[C@H](C)C(=O)O)ccc12 ; Cc1ccc(-c2c[nH]c(=O)c3cc(OCC#N)ccc23)c(Cl)c1 ; C[C@@H](Oc1ccc2c(-c3ccc(F)cc3Cl)c[nH]c(=O)c2c1)C(=O)N1CCC(F)(F)CC1]

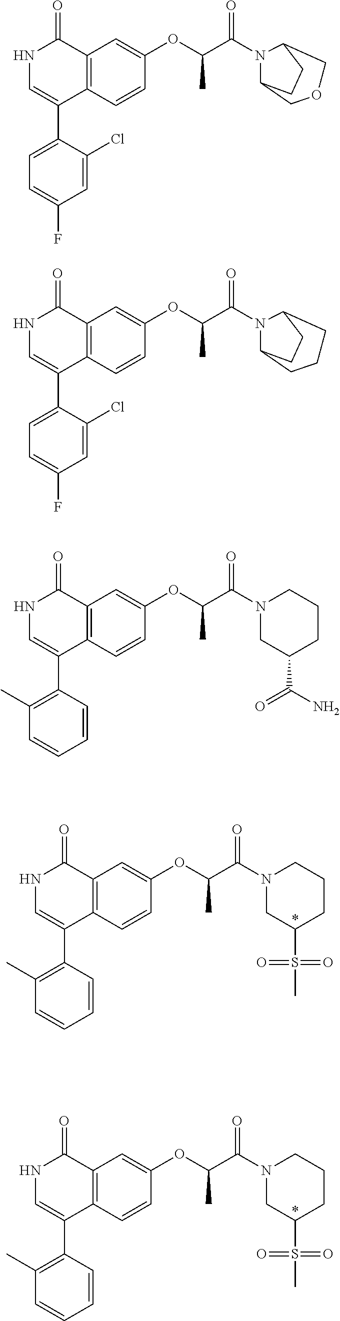

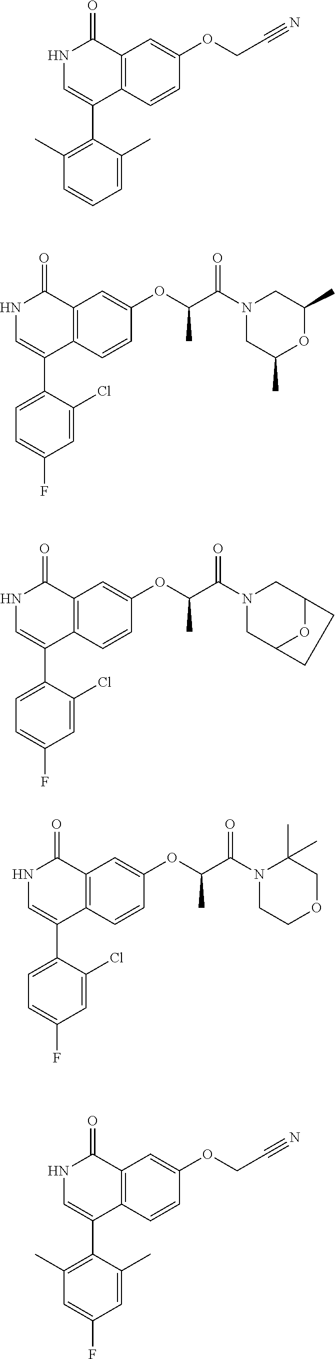

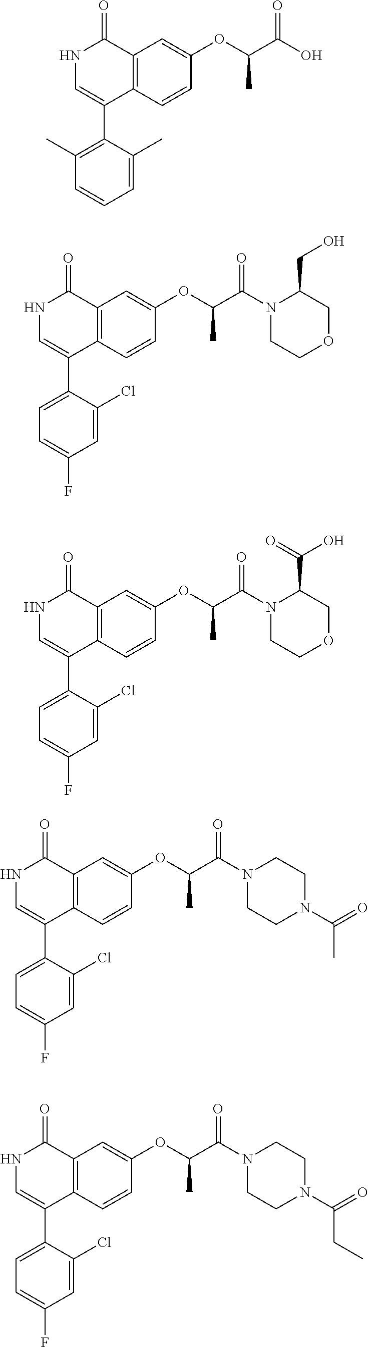

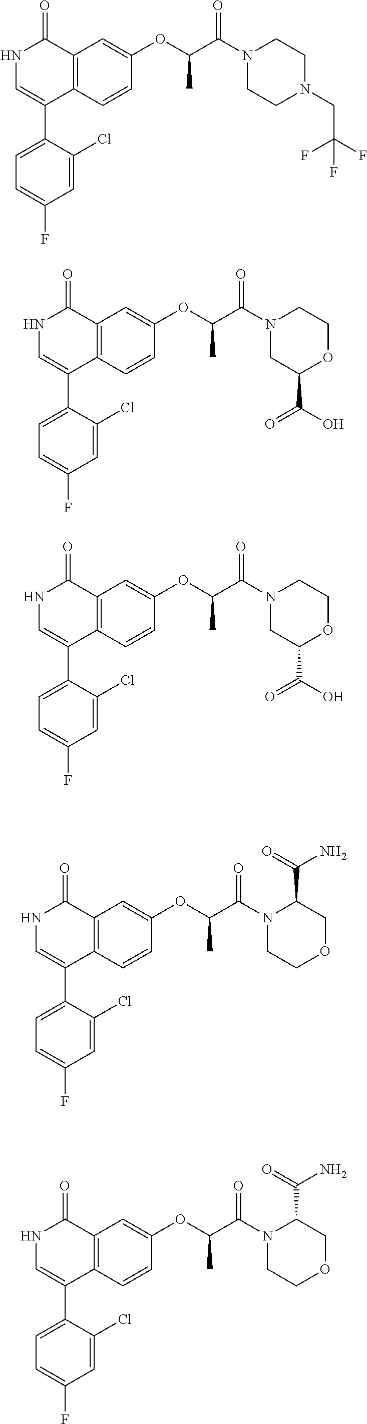

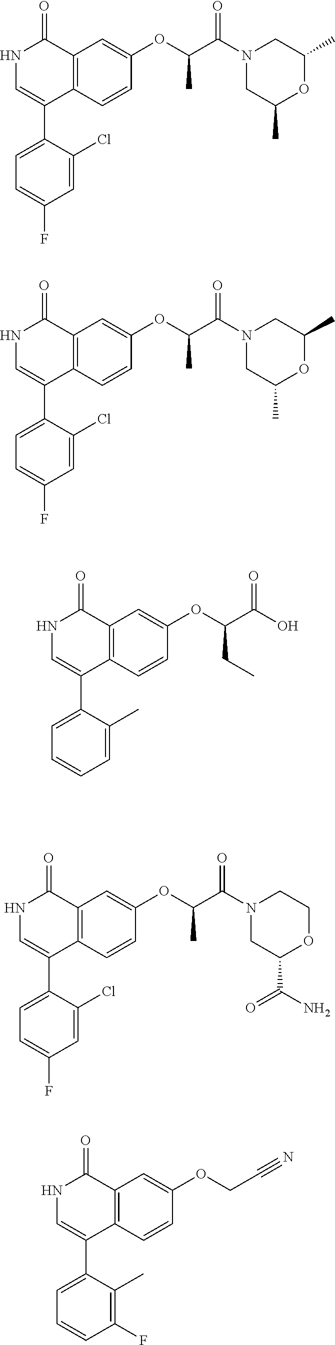

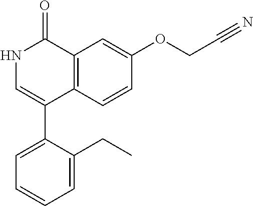 [SMILES: CCc1ccccc1-c1c[nH]c(=O)c2cc(OCC#N)ccc12]

11. The compound of claim 10, or a pharmaceutically acceptable salt thereof, wherein the compound is selected from the group consisting of:

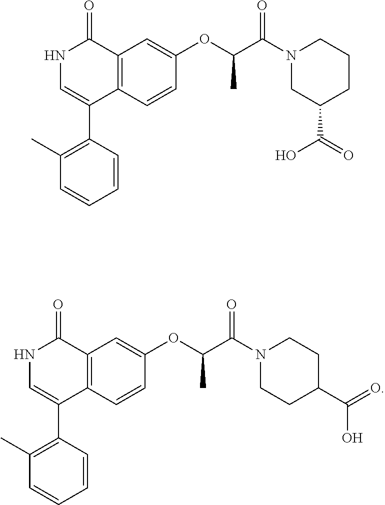 [SMILES: Cc1ccccc1-c1c[nH]c(=O)c2cc(O[C@H](C)C(=O)N3CCC[C@H](C(=O)O)C3)ccc12 ; Cc1ccccc1-c1c[nH]c(=O)c2cc(O[C@H](C)C(=O)N3CCC(C(=O)O)CC3)ccc12]

12. The compound of claim 10, or a pharmaceutically acceptable salt thereof, wherein the compound is selected from the group consisting of:

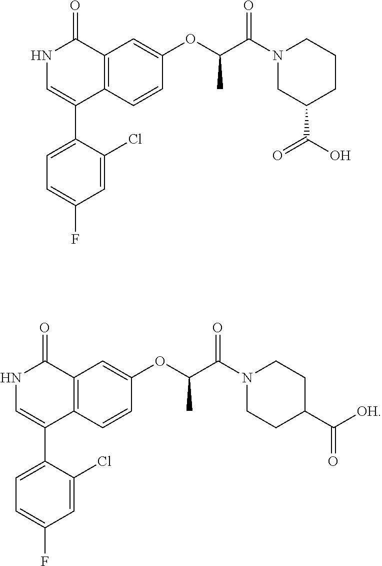 [SMILES: C[C@@H](Oc1ccc2c(-c3ccc(F)cc3Cl)c[nH]c(=O)c2c1)C(=O)N1CCC[C@H](C(=O)O)C1 ; C[C@@H](Oc1ccc2c(-c3ccc(F)cc3Cl)c[nH]c(=O)c2c1)C(=O)N1CCC(C(=O)O)CC1]

13. The compound of claim 10, or a pharmaceutically acceptable salt thereof, wherein the compound is selected from the group consisting of:

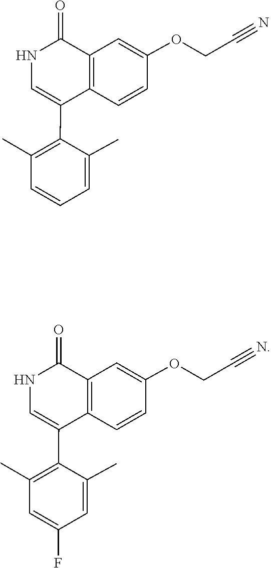 [SMILES: Cc1cccc(C)c1-c1c[nH]c(=O)c2cc(OCC#N)ccc12 ; Cc1cc(F)cc(C)c1-c1c[nH]c(=O)c2cc(OCC#N)ccc12]

14. The compound of claim 10, or a pharmaceutically acceptable salt thereof, wherein the compound is:

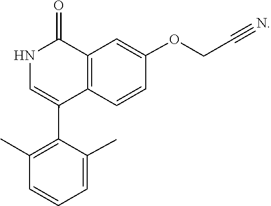 [SMILES: Cc1cccc(C)c1-c1c[nH]c(=O)c2cc(OCC#N)ccc12]

15. A pharmaceutical composition comprising the compound of claim 10, and a pharmaceutically acceptable excipient.

16. A method of inhibiting the activity of POLRMT with a compound of formula (I), or a pharmaceutically acceptable salt thereof: 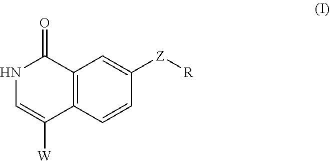
wherein:
Z is O, C(H)(R¹), C₆H₄, C(O), C(O)N(R⁷), or N(R²);
W is C₃-C₈ cycloalkyl, C₄-C₁₂ bicyclic, C₄-C₁₀ cycloalkenyl, C₆-C₁₂ aryl, or 5- to 12-membered heteroaryl, any of which is substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl;
R is hydrogen, or C₁-C₆ alkyl optionally substituted with one or more groups each independently selected from the group consisting of fluoro, hydroxyl, C₁-C₄ alkoxyl, cyano, C₁-C₄ haloalkoxyl, carboxyl, C(O)NR⁵R⁶, NR⁵R⁶ and NR²R³,
or R is C₆-C₁₂ aryl optionally substituted with one or more groups, each independently selected from the group consisting of fluoro, chloro, C₁-C₄ alkyl, trifluoromethyl, difluoromethyl, cyano, hydroxyl, NR⁵R⁶, and C₁-C₄ alkoxyl,
or R is hydroxyl, NR¹R², C(O)R³, C₁-C₄ haloalkoxyl, CH₂CH₂R⁸, CR³R⁴C(O)OR⁵, or 4-7 membered cyclic ring containing one or more heteroatoms, such cyclic ring is optionally substituted with C(O)R¹, NR¹—C(O)—R¹, or CR³R⁴C(O)NR⁵R⁶;
each R¹ is independently hydrogen, C₁-C₄ alkoxyl, or C₁-C₄ alkyl optionally substituted with one or more fluoro groups;
each R² is independently hydrogen, C₁-C₄ alkyl optionally substituted with one or more fluoro groups, C(O)C₁-C₄ alkyl optionally substituted with one or more fluoro or methoxyl groups, C(O)-cycloheteroalkyl optionally substituted with methyl or acetate, or C(O)NHC₁-C₄ alkyl optionally substituted with one or more fluoro groups;
R³ and R⁴ are each independently hydrogen or C₁-C₄ alkyl optionally substituted with one or more groups each independently selected from the group consisting of fluoro, hydroxyl, C₁-C₄ alkoxyl, C₁-C₄ haloalkoxyl, and NR⁵R⁶;
R⁵ and R⁶ are each independently hydrogen or C₁-C₄ alkyl optionally substituted with one or more groups each independently selected from the group consisting of fluoro, hydroxyl, cyano, C₁-C₄ alkoxyl, C₁-C₄ haloalkoxyl, and NR¹R²;
or if R⁵ and R⁶ are attached to the same nitrogen atom, R⁵ and R⁶ together with their connecting nitrogen form a 3- to 7-membered heterocyclic ring or 6- to 12-membered heterobicyclic ring, each such heterocyclic ring or heterobicyclic ring optionally containing another heteroatom that is N, O, S, S(O), SO₂R¹, or S(O)(NR¹) and each such heterocyclic ring or heterobicyclic ring is optionally substituted with one or more groups each independently selected from the group consisting of fluoro, chloro, and C₁-C₄ alkyl optionally substituted with one or more fluoro, C₁-alkoxyl or hydroxyl, and each such heterocyclic ring or heterobicyclic ring is further optionally substituted with cycloalkyl, cyano, carboxyl, C(O)R¹, C(O)NR¹R², imine, oxo, NR¹—C(O)—R¹, SO₂R¹, or C₁-C₄ alkylcarboxylate; and
R⁷ is H or C₁-C₆ alkyl; and
R⁸ is aryl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl, or R⁸ is OR¹, NR¹R², C(O)R¹, or C(O)NR²R².

17. The method of claim 16, wherein:
Z is O, C(H)(R¹), C₆H₄, C(O), C(O)N(R⁷), or N(R²);
W is C₆ cycloalkyl, C₅ bicyclic, C₆ cycloalkenyl, C₆ aryl or 5-membered heteroaryl, any of which is substituted with one or more groups, each independently selected from the group consisting of fluoro, chloro, cyano, and C₁-C₂ alkyl optionally substituted with one or more deuterium or hydroxyl;
R is hydrogen, or C₁-C₄ alkyl optionally substituted with one or more groups each independently selected from the group consisting of fluoro, hydroxyl, C₁ alkoxyl, cyano, carboxyl, C(O)NR⁵R⁶, and NR²R³,
or R is hydroxyl, NR¹R², C(O)R³, CR³R⁴C(O)OR⁵, or 4-7 membered cyclic ring containing one or more heteroatoms, such cyclic ring is optionally substituted with C(O)R¹, NR¹—C(O)—R¹, or CR³R⁴C(O)NR⁵R⁶;
each R¹ is independently hydrogen, C₁ alkoxyl, or C₁-C₂ alkyl;
each R² is independently hydrogen, C₁-C₂ alkyl optionally substituted with one or more fluoro groups, C(O)C₁-C₄ alkyl optionally substituted with methoxy, or C(O)-cycloheteroalkyl optionally substituted with methyl or acetate;
R³ and R⁴ are each independently hydrogen or C₁ alkyl optionally substituted with C₁ alkoxyl;
R⁵ and R⁶ are each independently hydrogen, C₁-C₂ alkyl, or cycloheteroalkyl substituted with acetate;
or if R⁵ and R⁶ are attached to the same nitrogen atom, R⁵ and R⁶ together with their connecting nitrogen form a 5- or 6-membered heterocyclic ring or 8-membered heterobicyclic ring, each such heterocyclic ring or heterobicyclic ring optionally containing another heteroatom that is N, O, S, S(O), SO₂R¹, or S(O)(NR¹) and each such heterocyclic ring or heterobicyclic ring is optionally substituted with one or more groups each independently selected from the group consisting of fluoro and C₁-C₂ alkyl optionally substituted with one or more fluoro, C₁-alkoxyl or hydroxyl, and each such heterocyclic ring or heterobicyclic ring is further optionally substituted with cyano, carboxyl, C(O)R¹, C(O)NR¹R², NR¹—C(O)—R¹, or SO₂R¹; and
R⁷ is C₁ alkyl.

18. The method of claim 17, wherein:
Z is O, or C₆H₄;
W is C₆ aryl substituted with one or more groups, each independently selected from the group consisting of fluoro, chloro, and C₁-C₂ alkyl;
R is hydrogen, C₁-C₄ alkyl optionally substituted with one or more groups each independently selected from the group consisting of fluoro, C₁ alkoxyl, cyano, carboxyl, and C(O)NR⁵R⁶;
each R¹ is independently hydrogen or C₁-C₂ alkyl;
each R² is independently hydrogen;
R⁵ and R⁶ are each independently hydrogen;
or if R⁵ and R⁶ are attached to the same nitrogen atom, R⁵ and R⁶ together with their connecting nitrogen form a 5- or 6-membered heterocyclic ring or 8-membered heterobicyclic ring, each such heterocyclic ring or heterobicyclic ring optionally containing another heteroatom that is N or O, and each such heterocyclic ring or heterobicyclic ring is optionally substituted with one or more groups each independently selected from the group consisting of fluoro and C₁-C₂ alkyl optionally substituted with one or more fluoro or hydroxyl, and each such heterocyclic ring or heterobicyclic ring is further optionally substituted with cyano, carboxyl, C(O)R¹, C(O)NR¹R², or SO₂R¹.

19. The method of claim 16, wherein the compound is selected from the group consisting of: 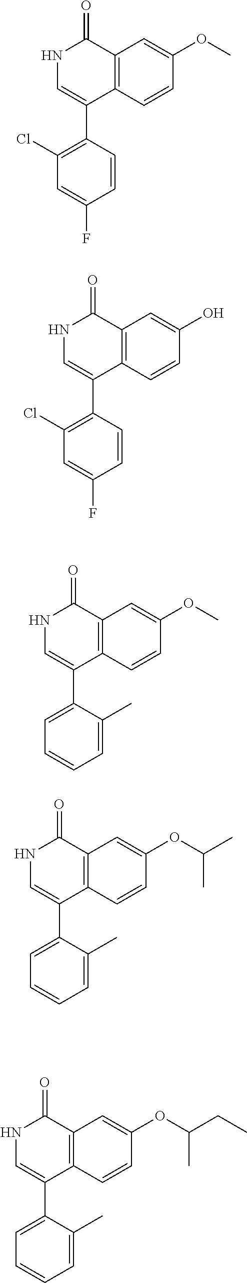 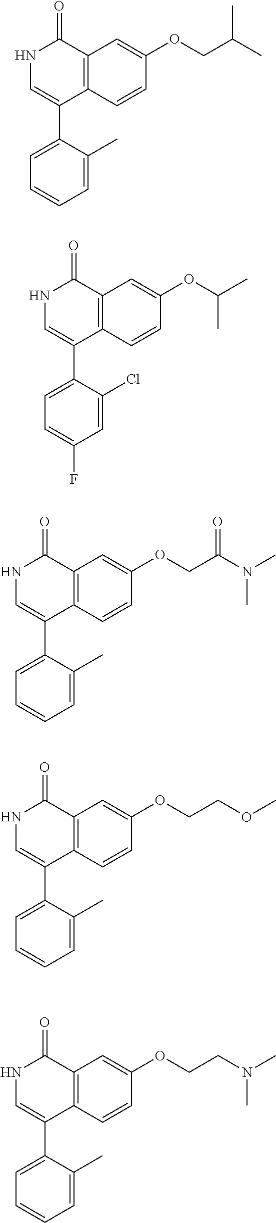 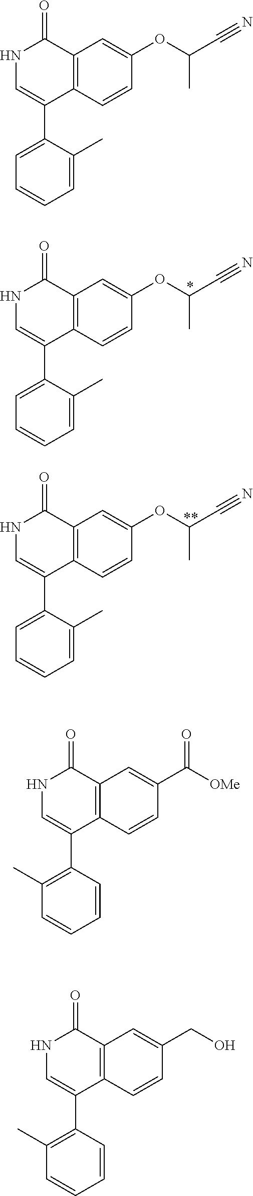 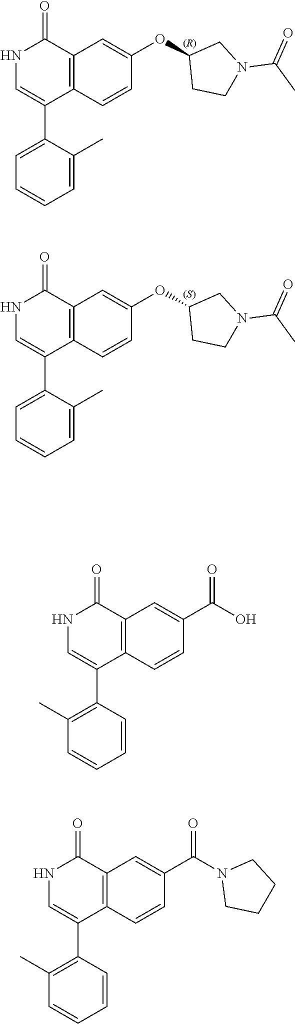 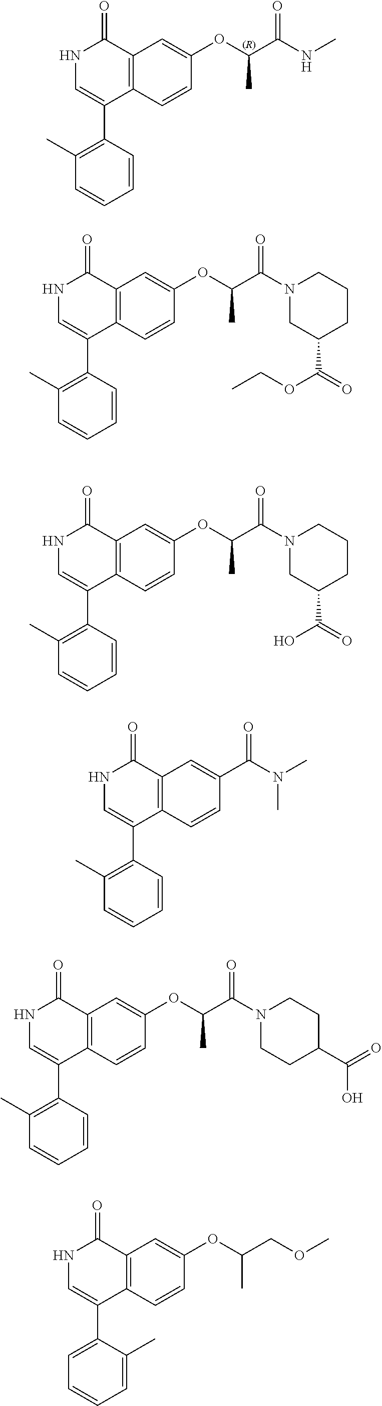 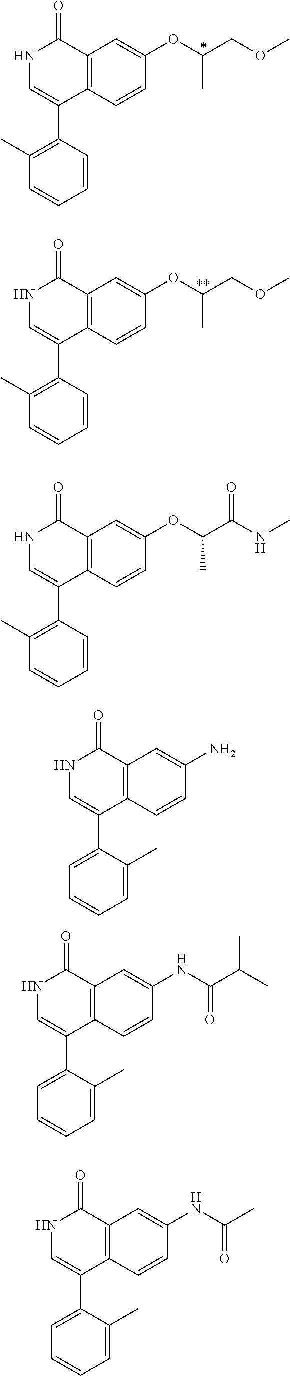 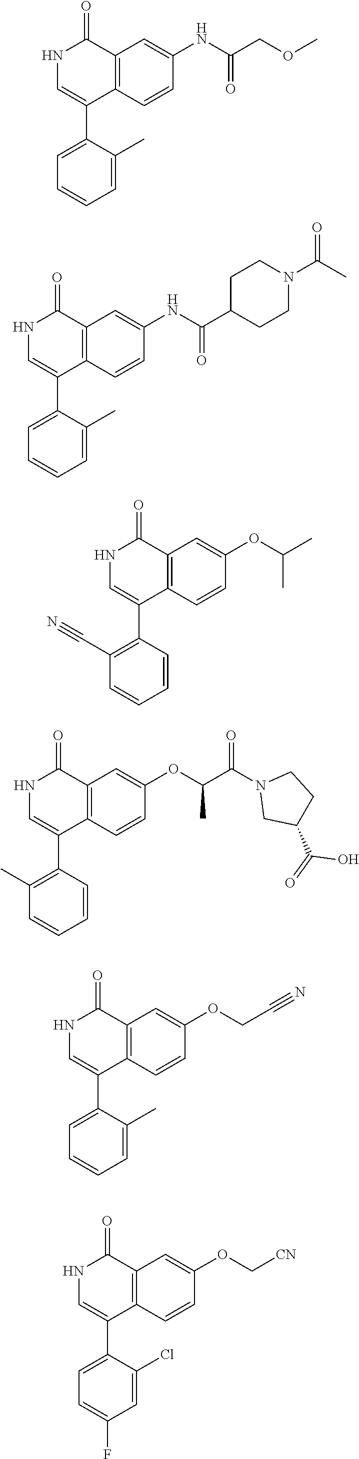 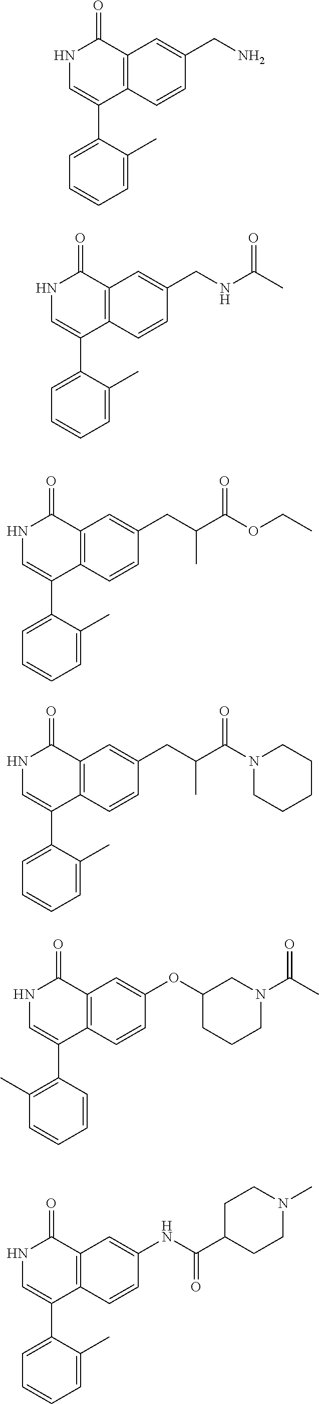 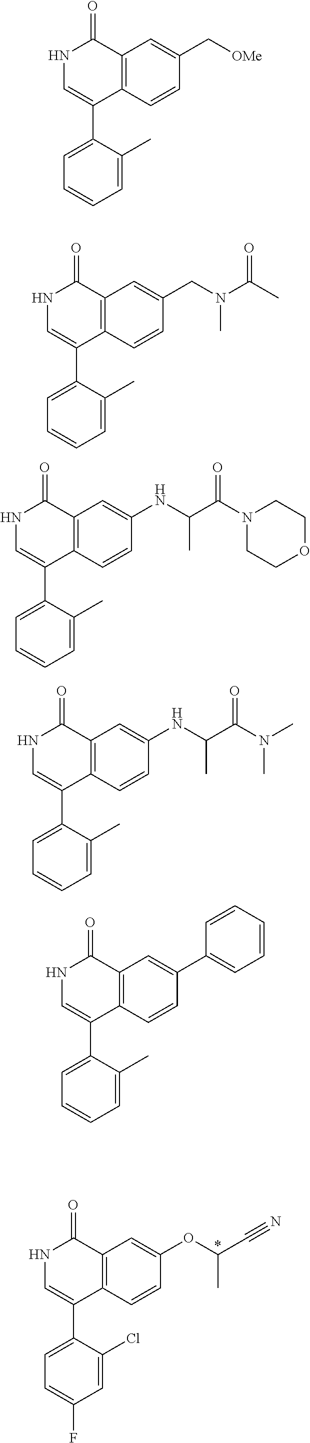 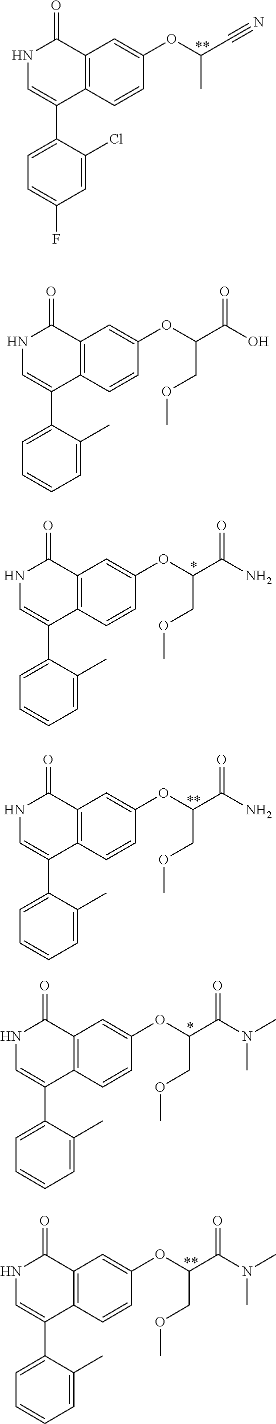 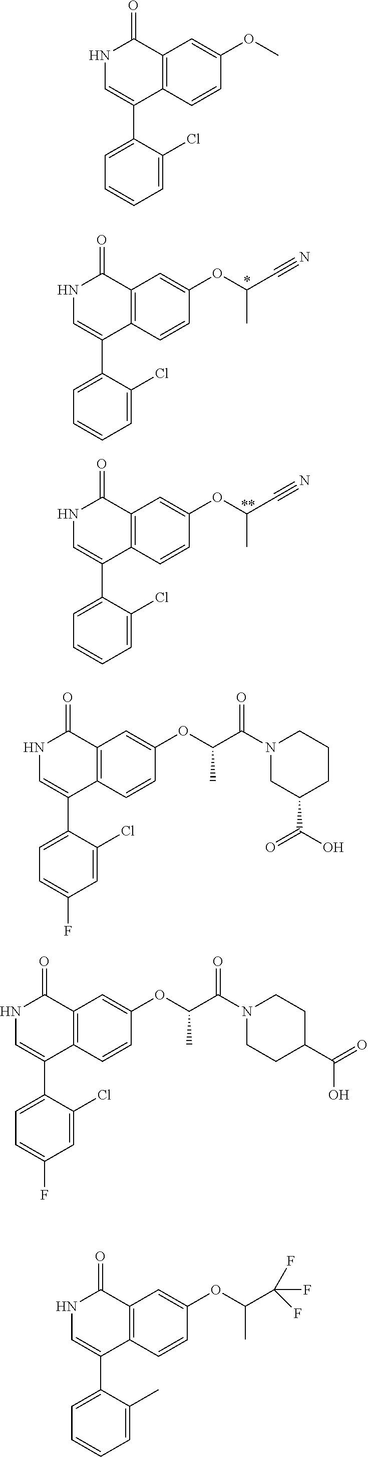 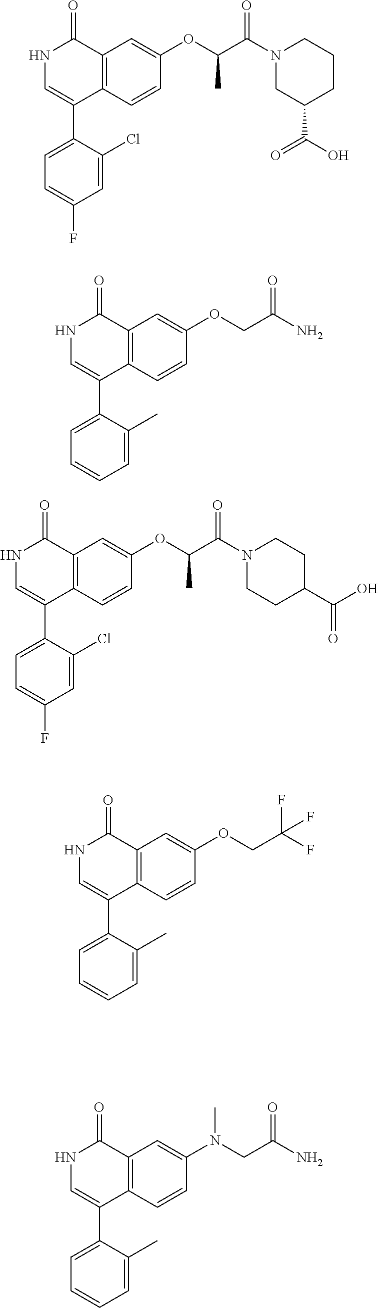 [SMILES: C[C@@H](Oc1ccc2c(-c3ccc(F)cc3Cl)c[nH]c(=O)c2c1)C(=O)N1CCC[C@H](C(=O)O)C1 ; Cc1ccccc1-c1c[nH]c(=O)c2cc(OCC(N)=O)ccc12 ; C[C@@H](Oc1ccc2c(-c3ccc(F)cc3Cl)c[nH]c(=O)c2c1)C(=O)N1CCC(C(=O)O)CC1 ; Cc1ccccc1-c1c[nH]c(=O)c2cc(OCC(F)(F)F)ccc12 ; Cc1ccccc1-c1c[nH]c(=O)c2cc(N(C)CC(N)=O)ccc12] 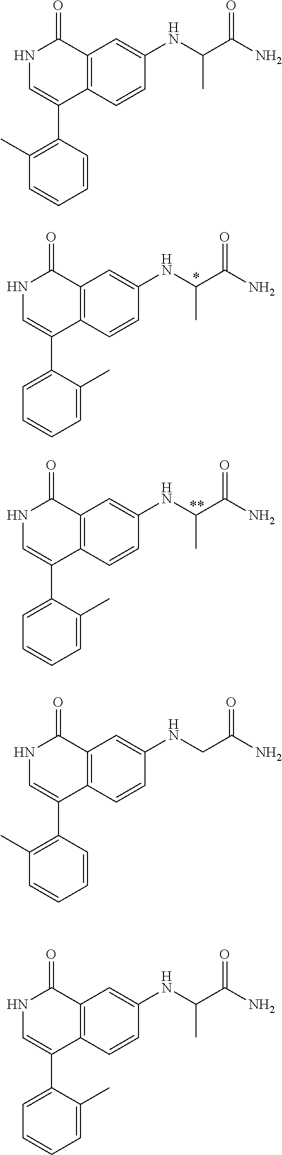 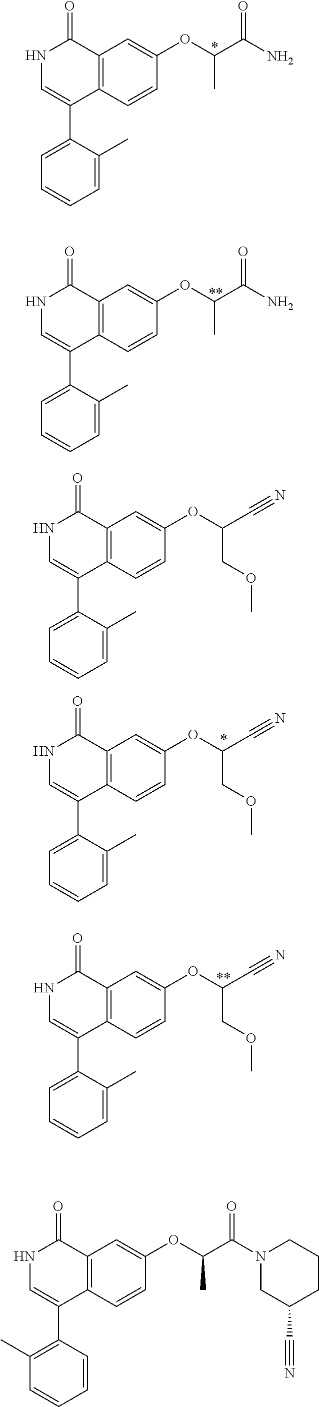 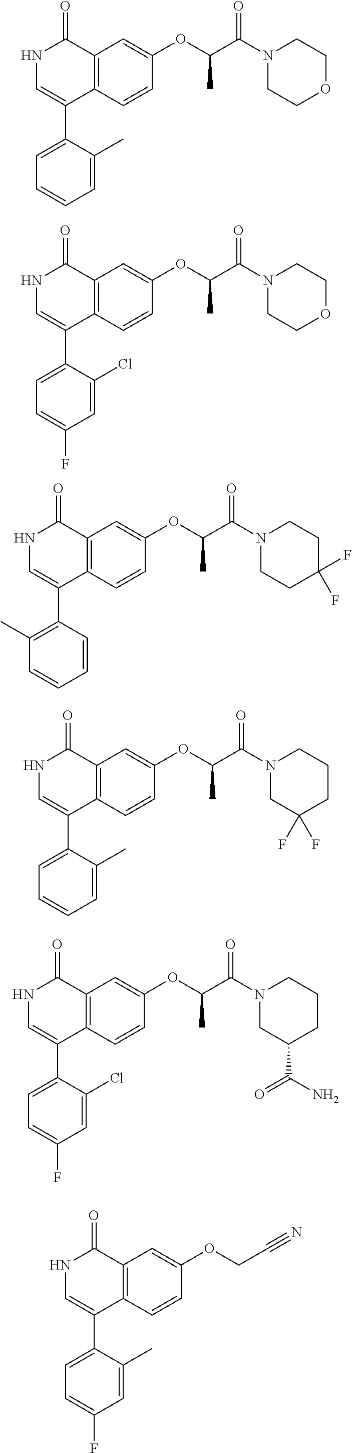 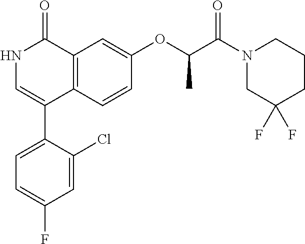 [SMILES: C[C@@H](Oc1ccc2c(-c3ccc(F)cc3Cl)c[nH]c(=O)c2c1)C(=O)N1CCCC(F)(F)C1] 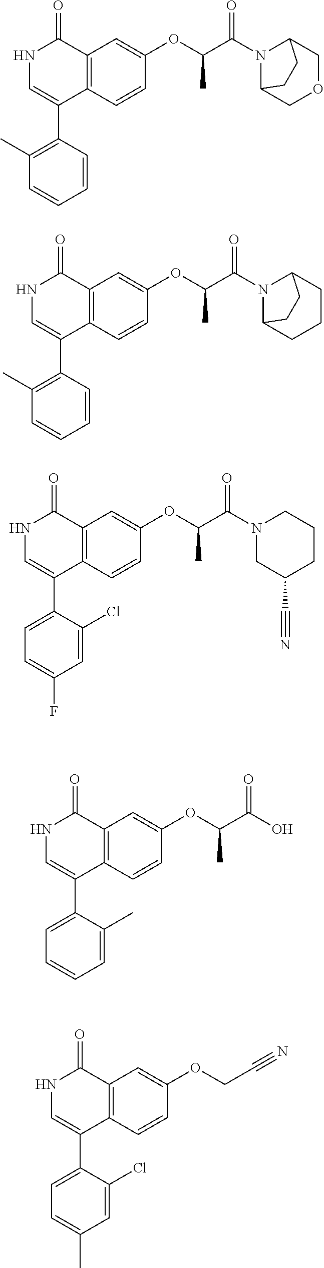 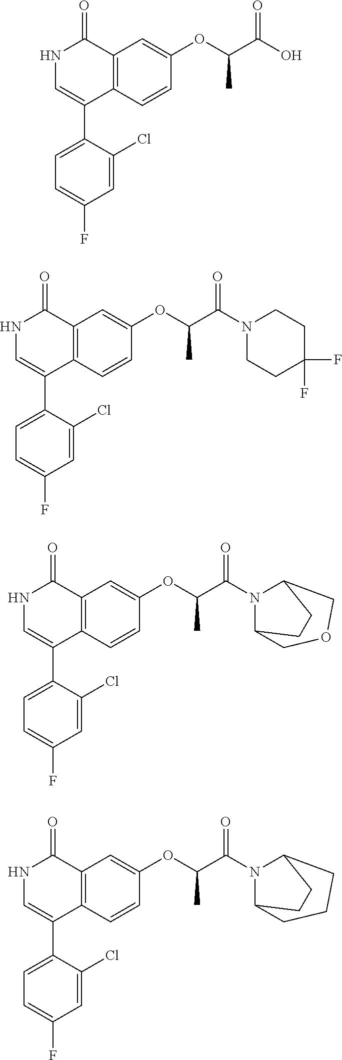 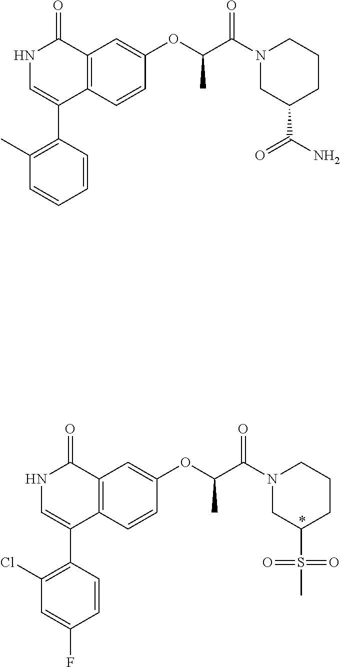 [SMILES: Cc1ccccc1-c1c[nH]c(=O)c2cc(O[C@H](C)C(=O)N3CCC[C@H](C(N)=O)C3)ccc12 ; C[C@@H](Oc1ccc2c(-c3ccc(F)cc3Cl)c[nH]c(=O)c2c1)C(=O)N1CCCC(S(C)(=O)=O)C1] 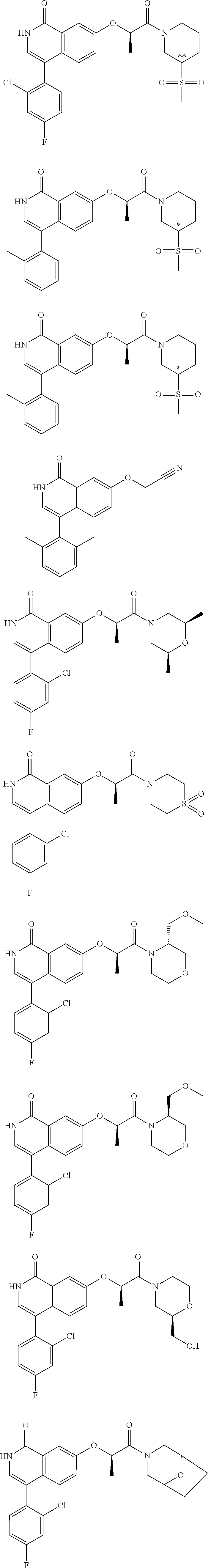 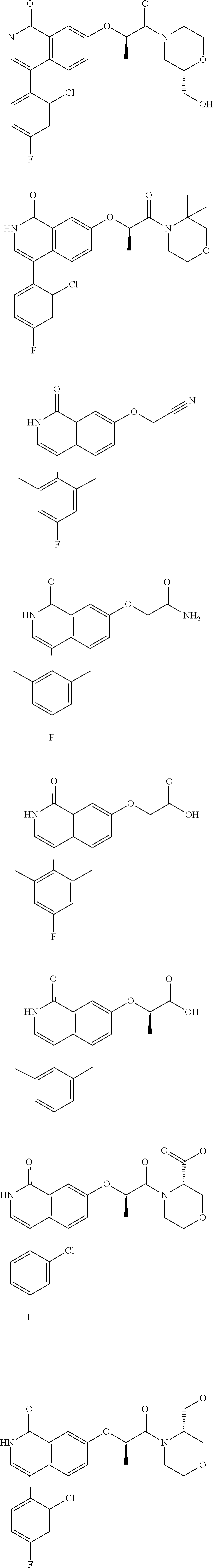 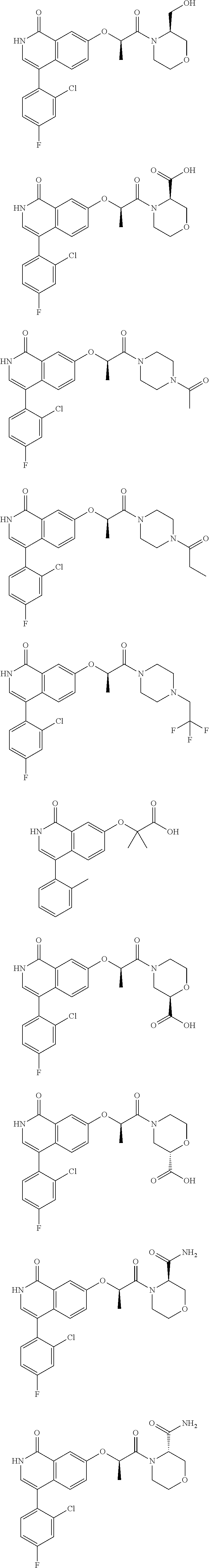 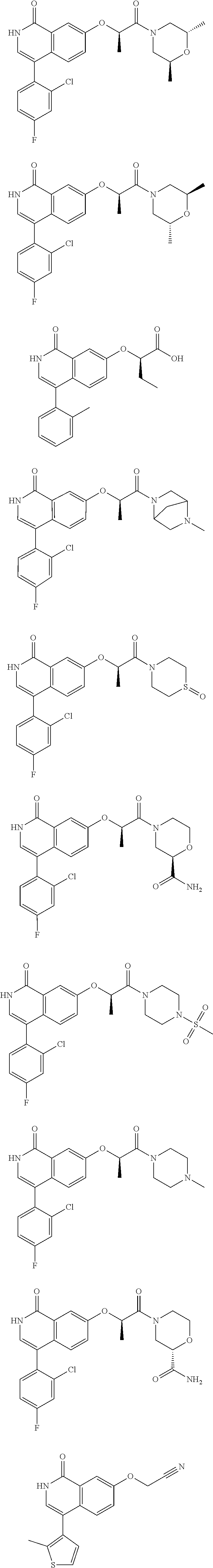 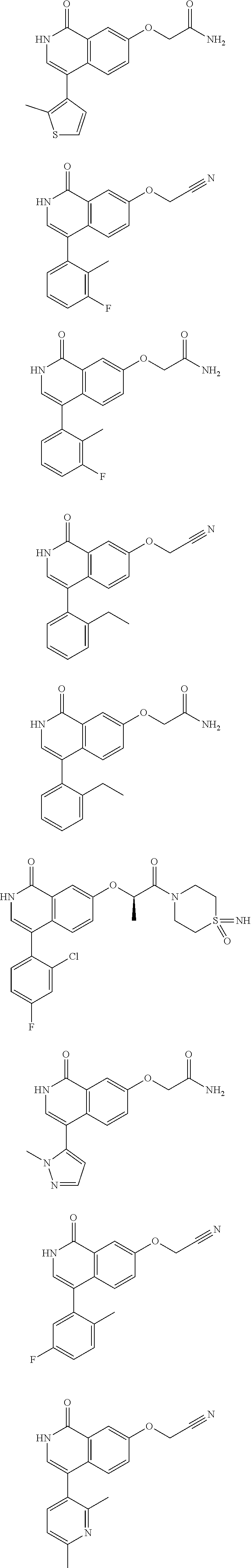 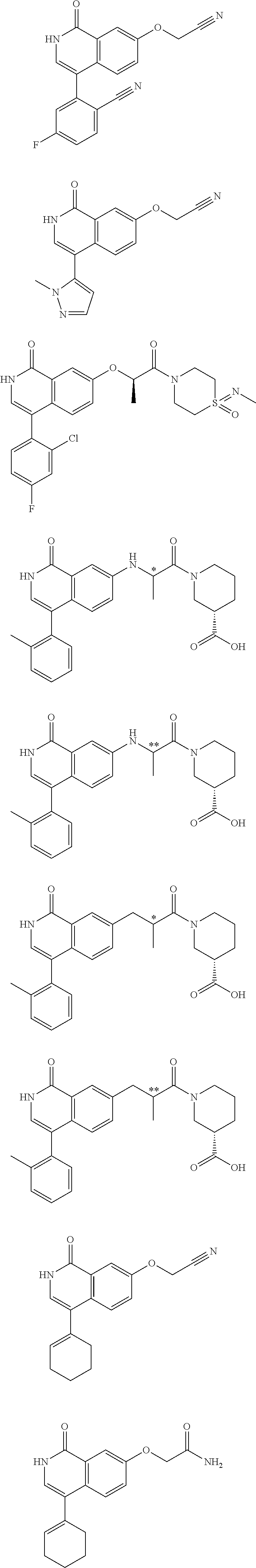 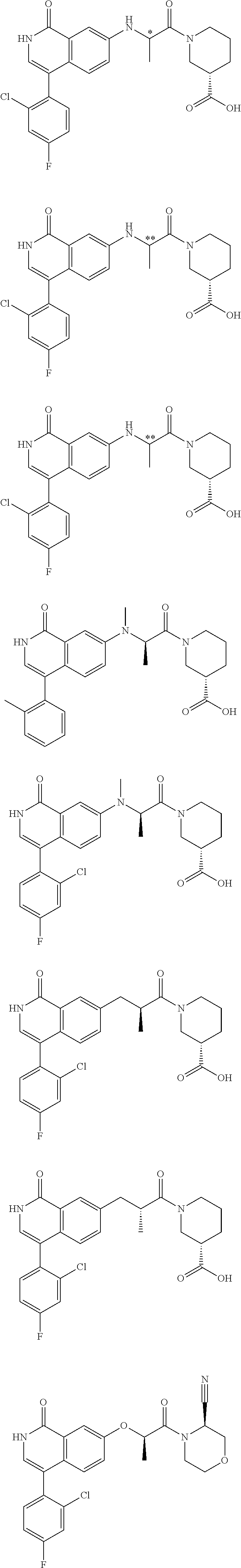 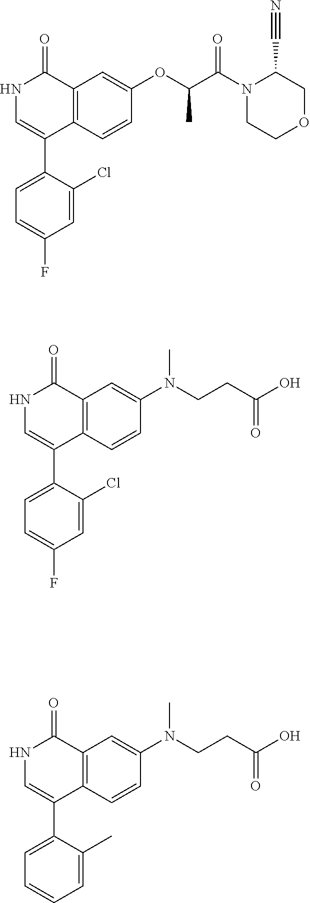 [SMILES: C[C@@H](Oc1ccc2c(-c3ccc(F)cc3Cl)c[nH]c(=O)c2c1)C(=O)N1CCOC[C@H]1C#N ; CN(CCC(=O)O)c1ccc2c(-c3ccc(F)cc3Cl)c[nH]c(=O)c2c1 ; Cc1ccccc1-c1c[nH]c(=O)c2cc(N(C)CCC(=O)O)ccc12] 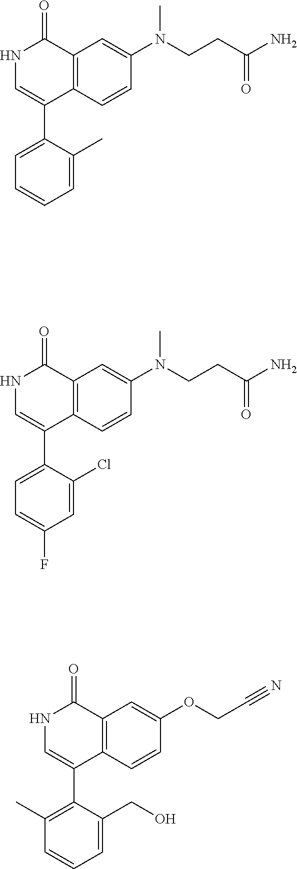 [SMILES: Cc1ccccc1-c1c[nH]c(=O)c2cc(N(C)CCC(N)=O)ccc12 ; CN(CCC(N)=O)c1ccc2c(-c3ccc(F)cc3Cl)c[nH]c(=O)c2c1 ; Cc1cccc(CO)c1-c1c[nH]c(=O)c2cc(OCC#N)ccc12] 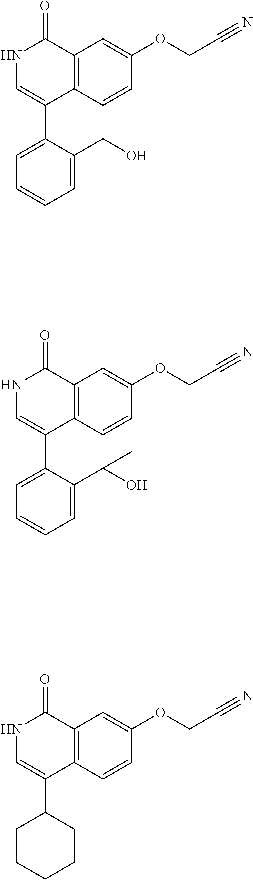 [SMILES: N#CCOc1ccc2c(-c3ccccc3CO)c[nH]c(=O)c2c1 ; CC(O)c1ccccc1-c1c[nH]c(=O)c2cc(OCC#N)ccc12 ; N#CCOc1ccc2c(C3CCCCC3)c[nH]c(=O)c2c1] 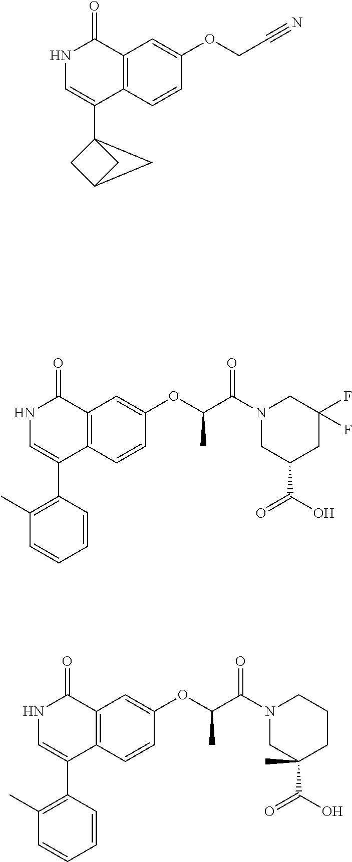 [SMILES: N#CCOc1ccc2c(C34CC(C3)C4)c[nH]c(=O)c2c1 ; Cc1ccccc1-c1c[nH]c(=O)c2cc(O[C@H](C)C(=O)N3C[C@@H](C(=O)O)CC(F)(F)C3)ccc12 ; Cc1ccccc1-c1c[nH]c(=O)c2cc(O[C@H](C)C(=O)N3CCC[C@](C)(C(=O)O)C3)ccc12] 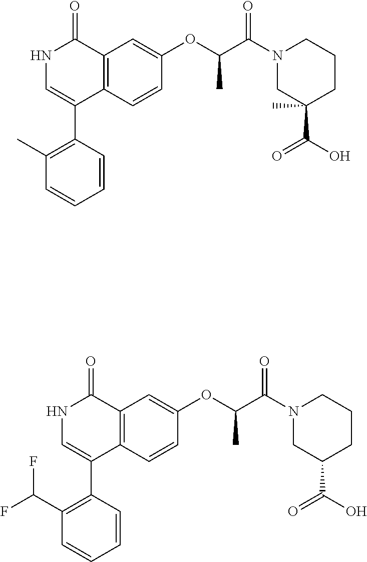 [SMILES: Cc1ccccc1-c1c[nH]c(=O)c2cc(O[C@H](C)C(=O)N3CCC[C@@](C)(C(=O)O)C3)ccc12 ; C[C@@H](Oc1ccc2c(-c3ccccc3C(F)F)c[nH]c(=O)c2c1)C(=O)N1CCC[C@H](C(=O)O)C1] 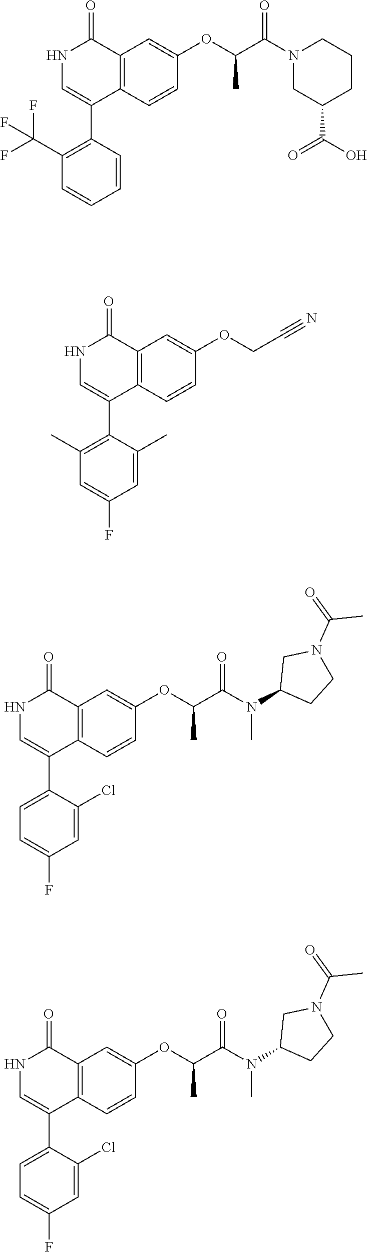 [SMILES: C[C@@H](Oc1ccc2c(-c3ccccc3C(F)(F)F)c[nH]c(=O)c2c1)C(=O)N1CCC[C@H](C(=O)O)C1 ; Cc1cc(F)cc(C)c1-c1c[nH]c(=O)c2cc(OCC#N)ccc12 ; CC(=O)N1CC[C@@H](N(C)C(=O)[C@@H](C)Oc2ccc3c(-c4ccc(F)cc4Cl)c[nH]c(=O)c3c2)C1 ; CC(=O)N1CC[C@H](N(C)C(=O)[C@@H](C)Oc2ccc3c(-c4ccc(F)cc4Cl)c[nH]c(=O)c3c2)C1] 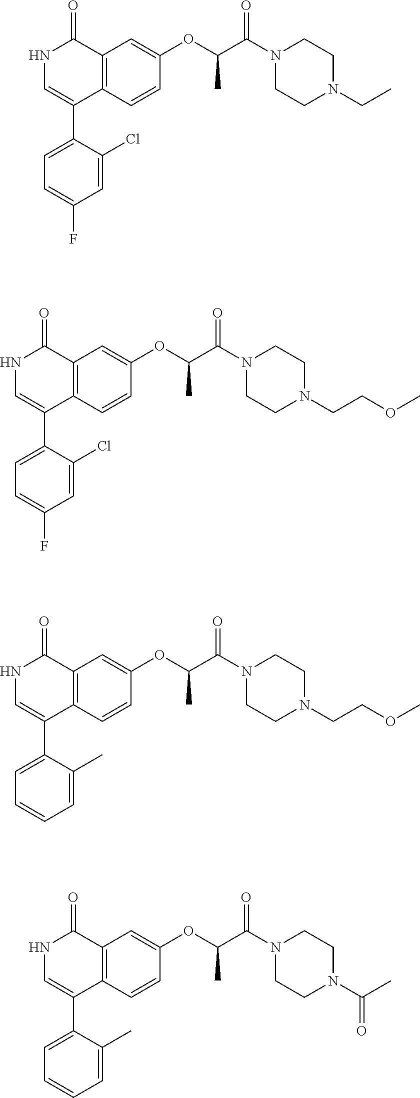 [SMILES: CCN1CCN(C(=O)[C@@H](C)Oc2ccc3c(-c4ccc(F)cc4Cl)c[nH]c(=O)c3c2)CC1 ; COCCN1CCN(C(=O)[C@@H](C)Oc2ccc3c(-c4ccc(F)cc4Cl)c[nH]c(=O)c3c2)CC1 ; COCCN1CCN(C(=O)[C@@H](C)Oc2ccc3c(-c4ccccc4C)c[nH]c(=O)c3c2)CC1 ; CC(=O)N1CCN(C(=O)[C@@H](C)Oc2ccc3c(-c4ccccc4C)c[nH]c(=O)c3c2)CC1] 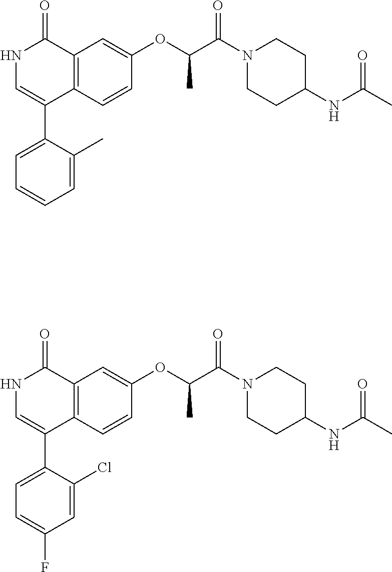 [SMILES: CC(=O)NC1CCN(C(=O)[C@@H](C)Oc2ccc3c(-c4ccccc4C)c[nH]c(=O)c3c2)CC1 ; CC(=O)NC1CCN(C(=O)[C@@H](C)Oc2ccc3c(-c4ccc(F)cc4Cl)c[nH]c(=O)c3c2)CC1] 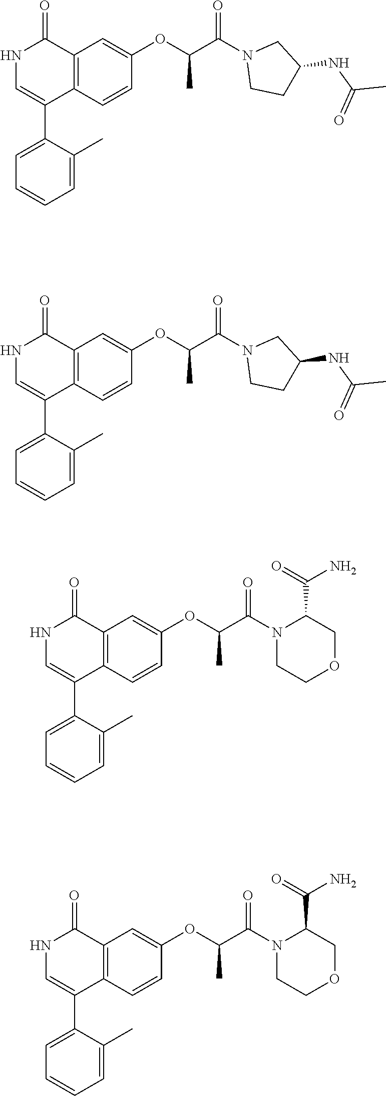 [SMILES: CC(=O)N[C@@H]1CCN(C(=O)[C@@H](C)Oc2ccc3c(-c4ccccc4C)c[nH]c(=O)c3c2)C1 ; CC(=O)N[C@H]1CCN(C(=O)[C@@H](C)Oc2ccc3c(-c4ccccc4C)c[nH]c(=O)c3c2)C1 ; Cc1ccccc1-c1c[nH]c(=O)c2cc(O[C@H](C)C(=O)N3CCOC[C@H]3C(N)=O)ccc12 ; Cc1ccccc1-c1c[nH]c(=O)c2cc(O[C@H](C)C(=O)N3CCOC[C@@H]3C(N)=O)ccc12] 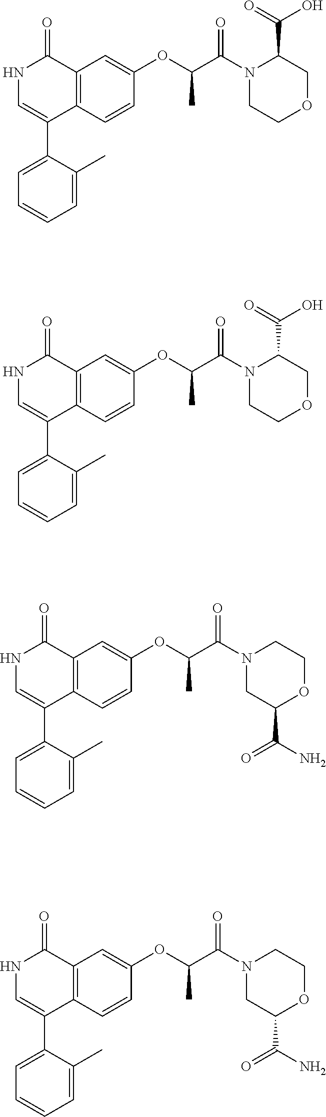 [SMILES: Cc1ccccc1-c1c[nH]c(=O)c2cc(O[C@H](C)C(=O)N3CCOC[C@@H]3C(=O)O)ccc12 ; Cc1ccccc1-c1c[nH]c(=O)c2cc(O[C@H](C)C(=O)N3CCOC[C@H]3C(=O)O)ccc12 ; Cc1ccccc1-c1c[nH]c(=O)c2cc(O[C@H](C)C(=O)N3CCO[C@@H](C(N)=O)C3)ccc12 ; Cc1ccccc1-c1c[nH]c(=O)c2cc(O[C@H](C)C(=O)N3CCO[C@H](C(N)=O)C3)ccc12] 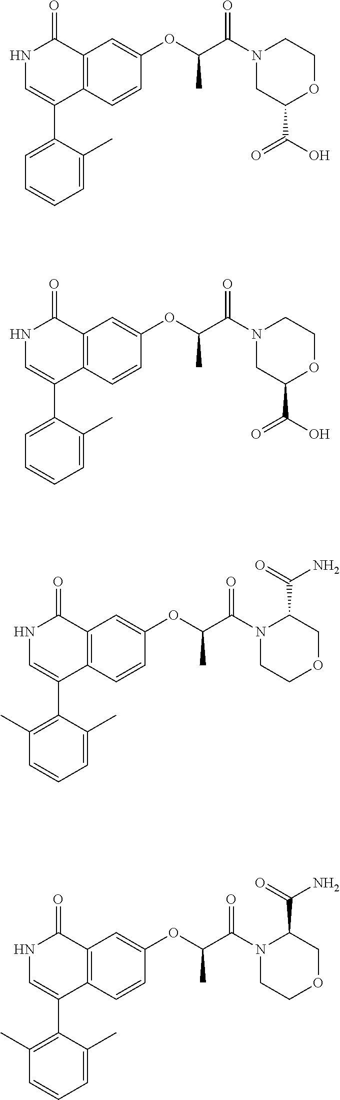 [SMILES: Cc1ccccc1-c1c[nH]c(=O)c2cc(O[C@H](C)C(=O)N3CCO[C@H](C(=O)O)C3)ccc12 ; Cc1ccccc1-c1c[nH]c(=O)c2cc(O[C@H](C)C(=O)N3CCO[C@@H](C(=O)O)C3)ccc12 ; Cc1cccc(C)c1-c1c[nH]c(=O)c2cc(O[C@H](C)C(=O)N3CCOC[C@H]3C(N)=O)ccc12 ; Cc1cccc(C)c1-c1c[nH]c(=O)c2cc(O[C@H](C)C(=O)N3CCOC[C@@H]3C(N)=O)ccc12] 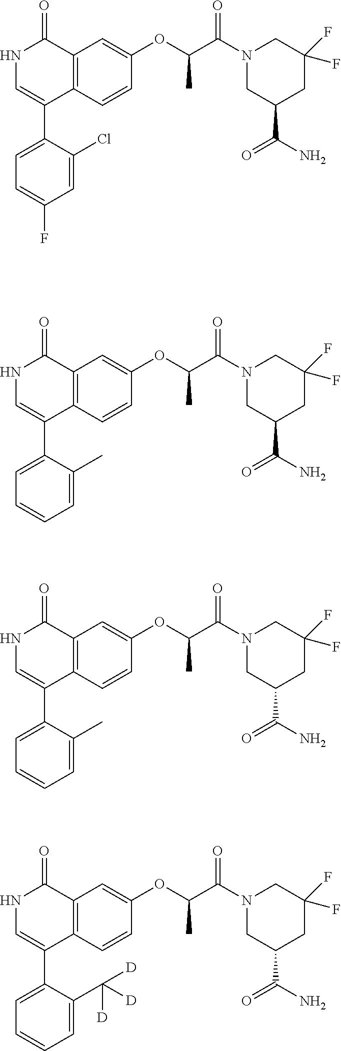 [SMILES: C[C@@H](Oc1ccc2c(-c3ccc(F)cc3Cl)c[nH]c(=O)c2c1)C(=O)N1C[C@H](C(N)=O)CC(F)(F)C1 ; Cc1ccccc1-c1c[nH]c(=O)c2cc(O[C@H](C)C(=O)N3C[C@H](C(N)=O)CC(F)(F)C3)ccc12 ; Cc1ccccc1-c1c[nH]c(=O)c2cc(O[C@H](C)C(=O)N3C[C@@H](C(N)=O)CC(F)(F)C3)ccc12 ; [2H]C([2H])([2H])c1ccccc1-c1c[nH]c(=O)c2cc(O[C@H](C)C(=O)N3C[C@@H](C(N)=O)CC(F)(F)C3)ccc12] 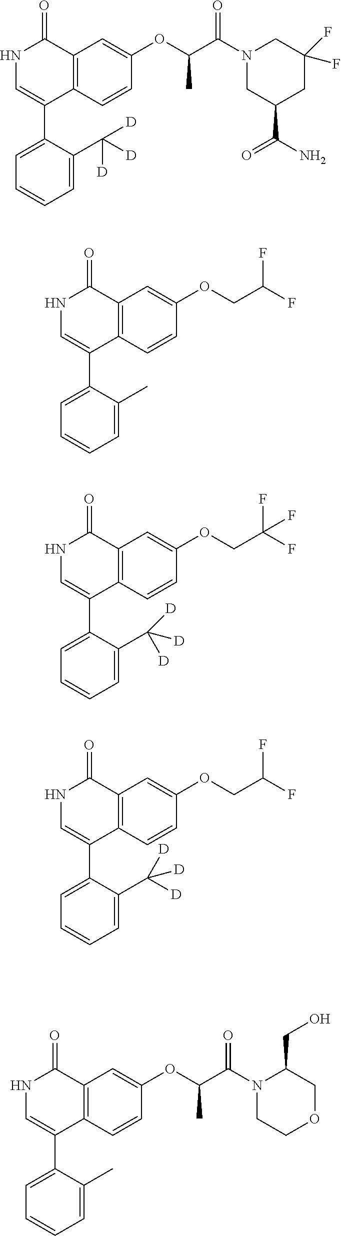 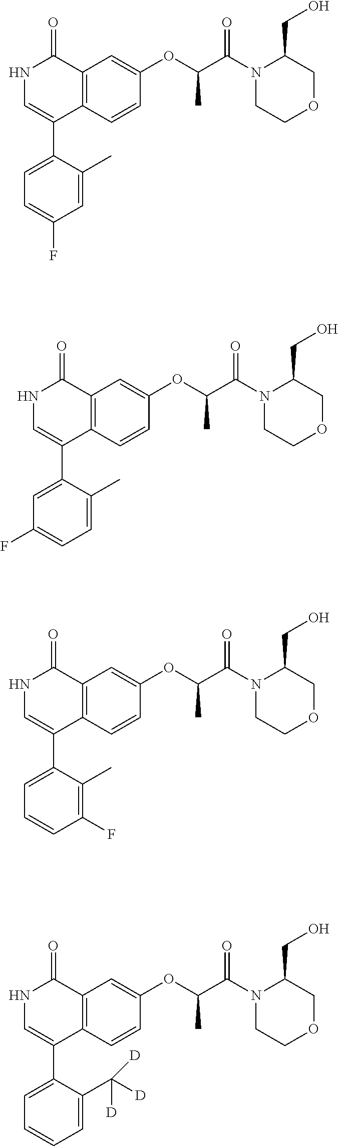 [SMILES: Cc1cc(F)ccc1-c1c[nH]c(=O)c2cc(O[C@H](C)C(=O)N3CCOC[C@@H]3CO)ccc12 ; Cc1ccc(F)cc1-c1c[nH]c(=O)c2cc(O[C@H](C)C(=O)N3CCOC[C@@H]3CO)ccc12 ; Cc1c(F)cccc1-c1c[nH]c(=O)c2cc(O[C@H](C)C(=O)N3CCOC[C@@H]3CO)ccc12 ; [2H]C([2H])([2H])c1ccccc1-c1c[nH]c(=O)c2cc(O[C@H](C)C(=O)N3CCOC[C@@H]3CO)ccc12] 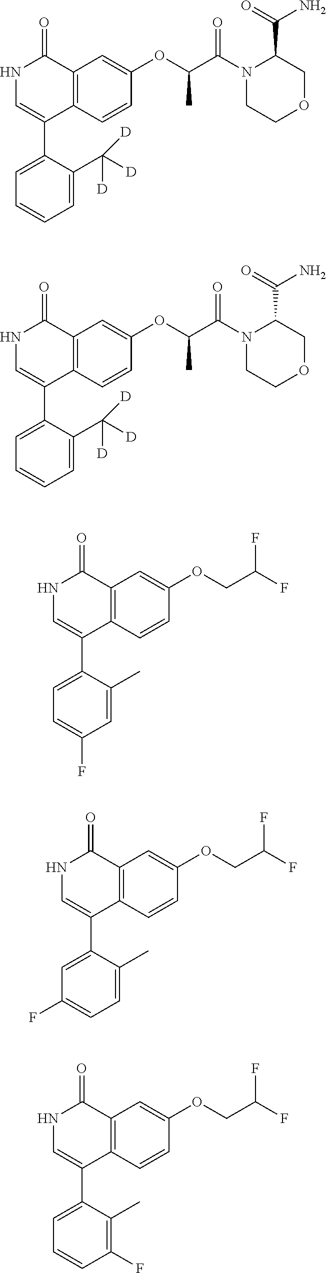 [SMILES: [2H]C([2H])([2H])c1ccccc1-c1c[nH]c(=O)c2cc(O[C@H](C)C(=O)N3CCOC[C@@H]3C(N)=O)ccc12 ; [2H]C([2H])([2H])c1ccccc1-c1c[nH]c(=O)c2cc(O[C@H](C)C(=O)N3CCOC[C@H]3C(N)=O)ccc12 ; Cc1cc(F)ccc1-c1c[nH]c(=O)c2cc(OCC(F)F)ccc12 ; Cc1ccc(F)cc1-c1c[nH]c(=O)c2cc(OCC(F)F)ccc12 ; Cc1c(F)cccc1-c1c[nH]c(=O)c2cc(OCC(F)F)ccc12] 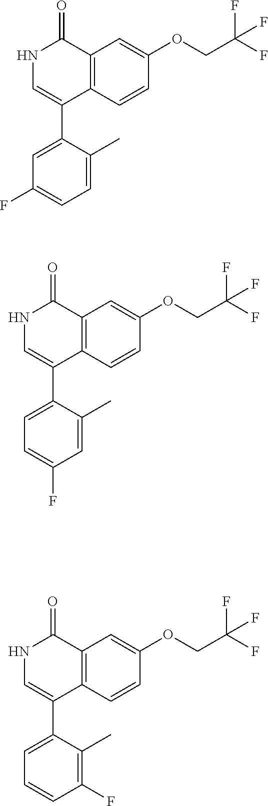 [SMILES: Cc1ccc(F)cc1-c1c[nH]c(=O)c2cc(OCC(F)(F)F)ccc12 ; Cc1cc(F)ccc1-c1c[nH]c(=O)c2cc(OCC(F)(F)F)ccc12 ; Cc1c(F)cccc1-c1c[nH]c(=O)c2cc(OCC(F)(F)F)ccc12] 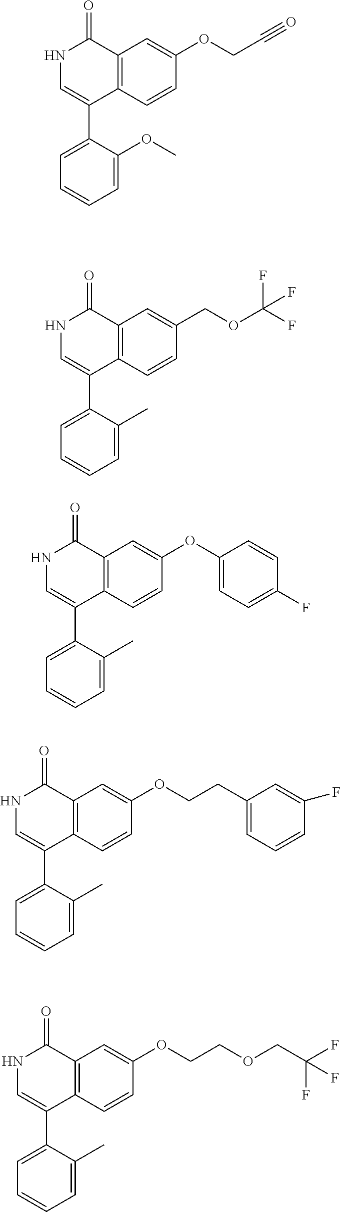 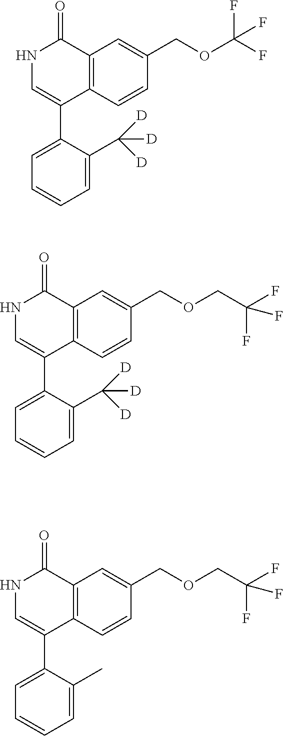 [SMILES: [2H]C([2H])([2H])c1ccccc1-c1c[nH]c(=O)c2cc(COC(F)(F)F)ccc12 ; [2H]C([2H])([2H])c1ccccc1-c1c[nH]c(=O)c2cc(COCC(F)(F)F)ccc12 ; Cc1ccccc1-c1c[nH]c(=O)c2cc(COCC(F)(F)F)ccc12]
or a pharmaceutically acceptable salt thereof.

20. The method of claim 19, wherein the compound is selected from the group consisting of: 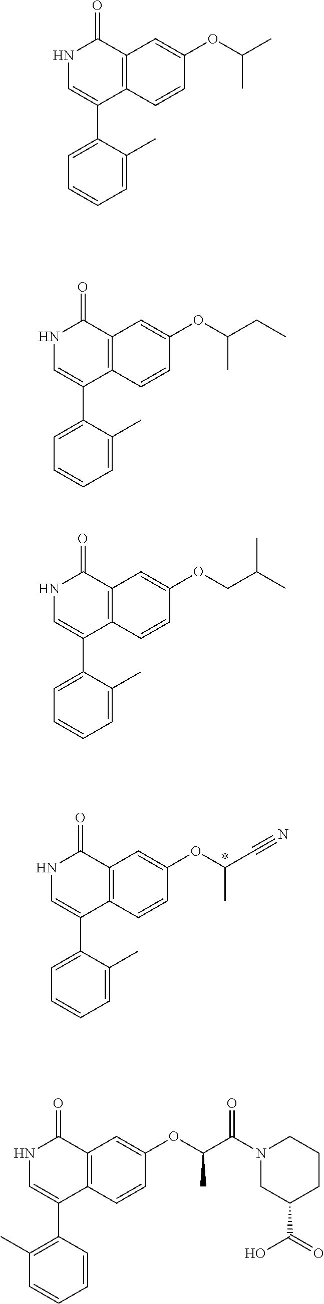 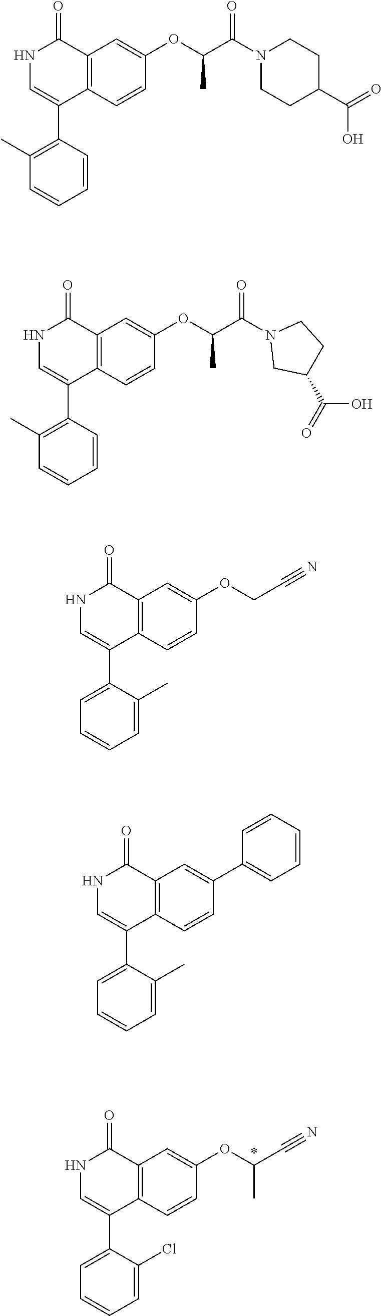 [SMILES: Cc1ccccc1-c1c[nH]c(=O)c2cc(O[C@H](C)C(=O)N3CCC(C(=O)O)CC3)ccc12 ; Cc1ccccc1-c1c[nH]c(=O)c2cc(O[C@H](C)C(=O)N3CC[C@H](C(=O)O)C3)ccc12 ; Cc1ccccc1-c1c[nH]c(=O)c2cc(OCC#N)ccc12 ; Cc1ccccc1-c1c[nH]c(=O)c2cc(-c3ccccc3)ccc12 ; CC(C#N)Oc1ccc2c(-c3ccccc3Cl)c[nH]c(=O)c2c1] 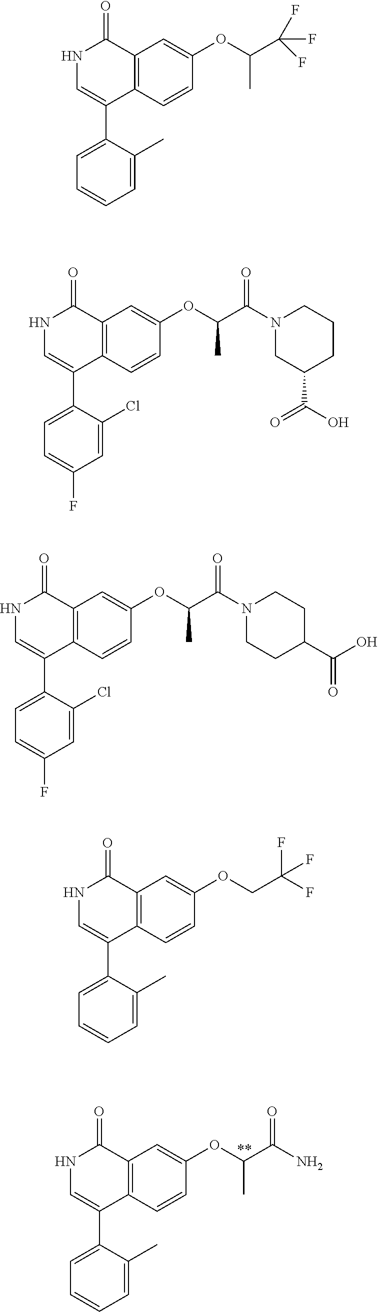 [SMILES: Cc1ccccc1-c1c[nH]c(=O)c2cc(OC(C)C(F)(F)F)ccc12 ; C[C@@H](Oc1ccc2c(-c3ccc(F)cc3Cl)c[nH]c(=O)c2c1)C(=O)N1CCC[C@H](C(=O)O)C1 ; C[C@@H](Oc1ccc2c(-c3ccc(F)cc3Cl)c[nH]c(=O)c2c1)C(=O)N1CCC(C(=O)O)CC1 ; Cc1ccccc1-c1c[nH]c(=O)c2cc(OCC(F)(F)F)ccc12 ; Cc1ccccc1-c1c[nH]c(=O)c2cc(OC(C)C(N)=O)ccc12] 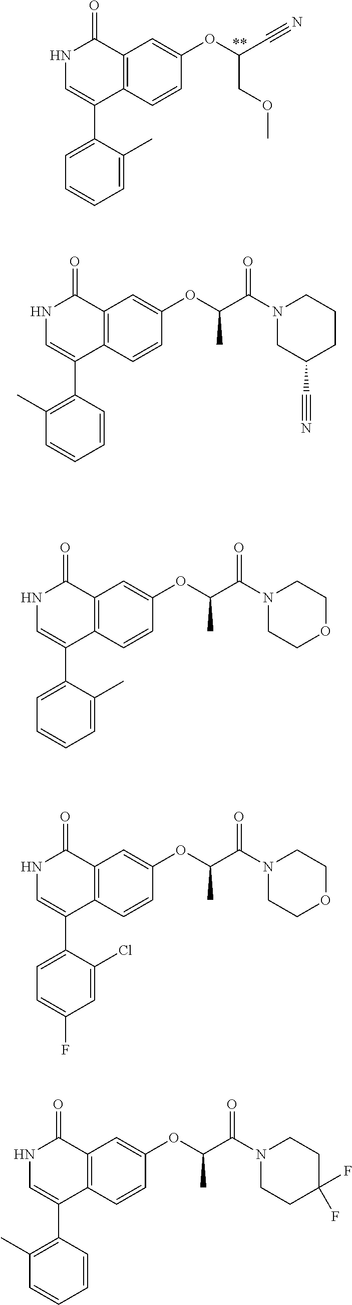 [SMILES: COCC(C#N)Oc1ccc2c(-c3ccccc3C)c[nH]c(=O)c2c1 ; Cc1ccccc1-c1c[nH]c(=O)c2cc(O[C@H](C)C(=O)N3CCC[C@H](C#N)C3)ccc12 ; Cc1ccccc1-c1c[nH]c(=O)c2cc(O[C@H](C)C(=O)N3CCOCC3)ccc12 ; C[C@@H](Oc1ccc2c(-c3ccc(F)cc3Cl)c[nH]c(=O)c2c1)C(=O)N1CCOCC1 ; Cc1ccccc1-c1c[nH]c(=O)c2cc(O[C@H](C)C(=O)N3CCC(F)(F)CC3)ccc12] 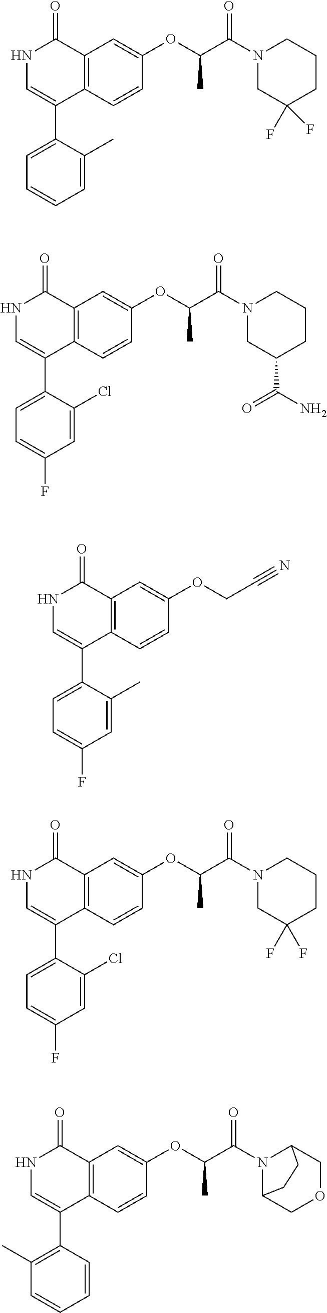 [SMILES: Cc1ccccc1-c1c[nH]c(=O)c2cc(O[C@H](C)C(=O)N3CCCC(F)(F)C3)ccc12 ; C[C@@H](Oc1ccc2c(-c3ccc(F)cc3Cl)c[nH]c(=O)c2c1)C(=O)N1CCC[C@H](C(N)=O)C1 ; Cc1cc(F)ccc1-c1c[nH]c(=O)c2cc(OCC#N)ccc12 ; C[C@@H](Oc1ccc2c(-c3ccc(F)cc3Cl)c[nH]c(=O)c2c1)C(=O)N1CCCC(F)(F)C1 ; Cc1ccccc1-c1c[nH]c(=O)c2cc(O[C@H](C)C(=O)N3C4CCC3COC4)ccc12] 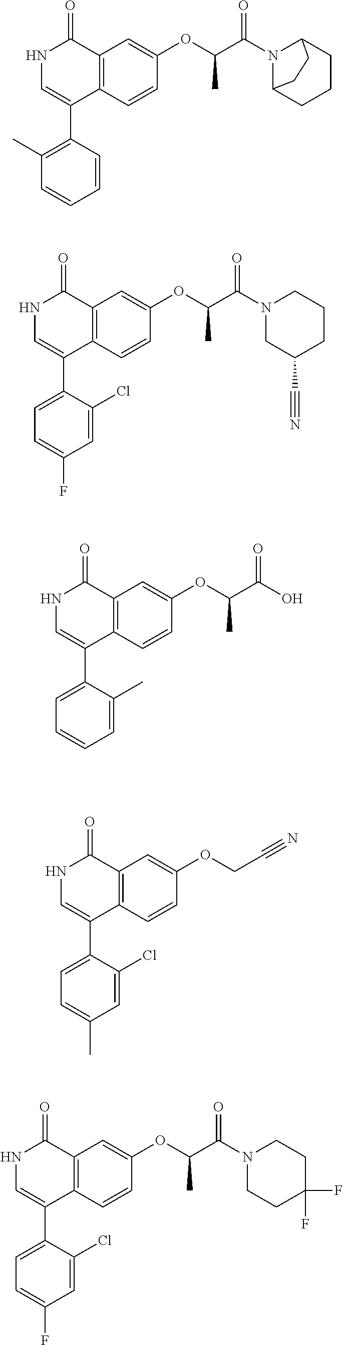 [SMILES: Cc1ccccc1-c1c[nH]c(=O)c2cc(O[C@H](C)C(=O)N3C4CCCC3CC4)ccc12 ; C[C@@H](Oc1ccc2c(-c3ccc(F)cc3Cl)c[nH]c(=O)c2c1)C(=O)N1CCC[C@H](C#N)C1 ; Cc1ccccc1-c1c[nH]c(=O)c2cc(O[C@H](C)C(=O)O)ccc12 ; Cc1ccc(-c2c[nH]c(=O)c3cc(OCC#N)ccc23)c(Cl)c1 ; C[C@@H](Oc1ccc2c(-c3ccc(F)cc3Cl)c[nH]c(=O)c2c1)C(=O)N1CCC(F)(F)CC1] 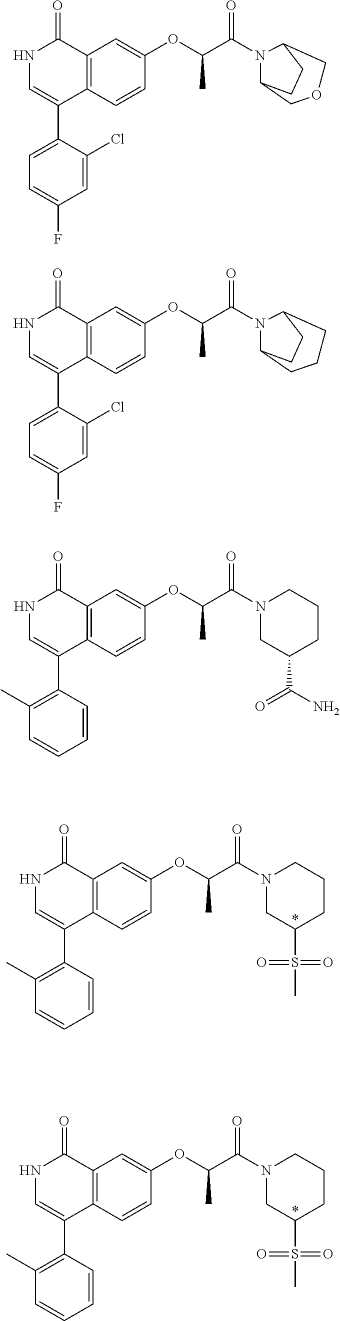 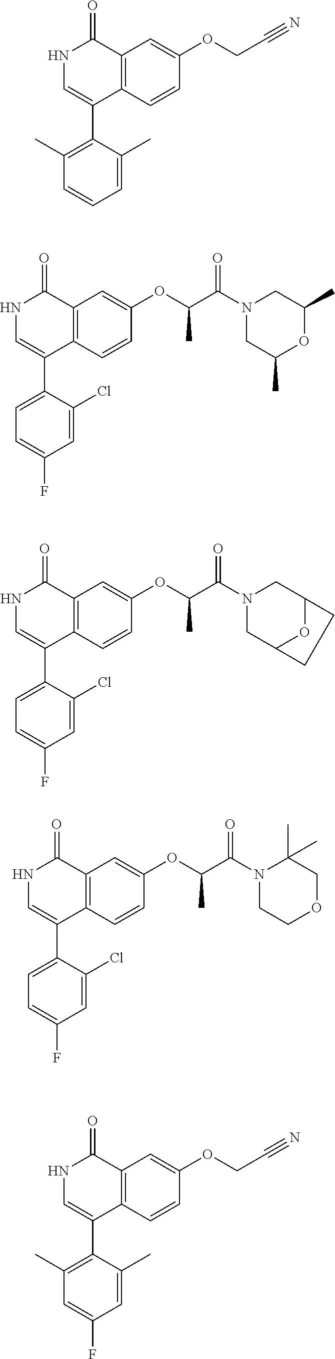 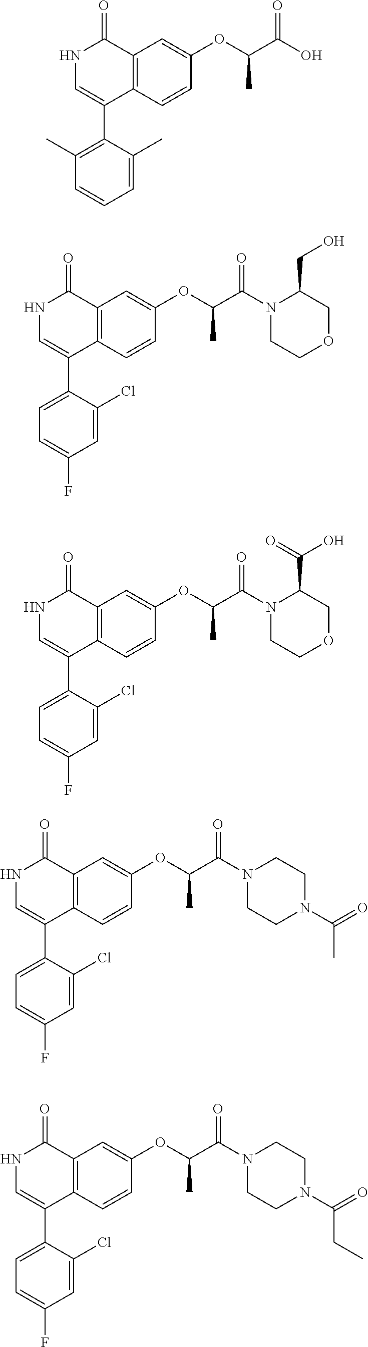 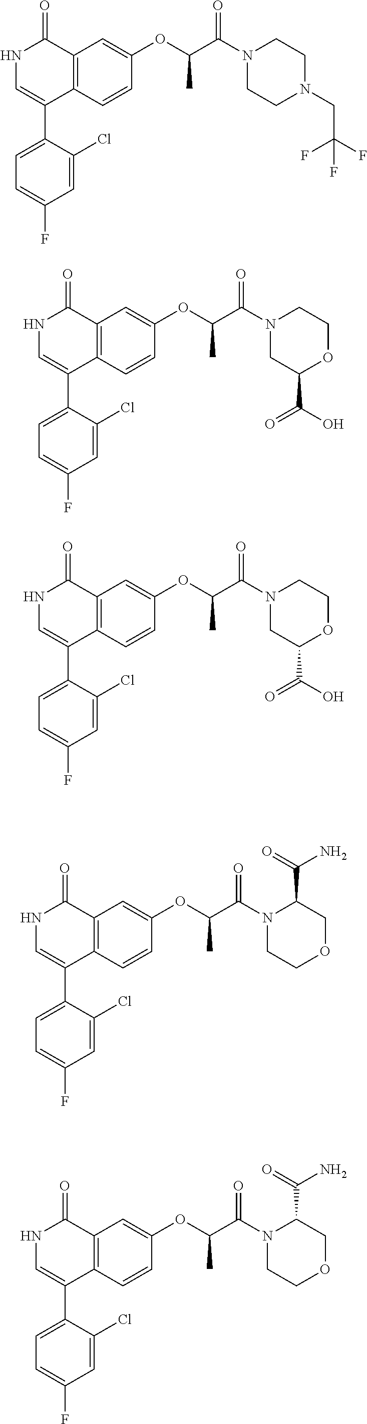 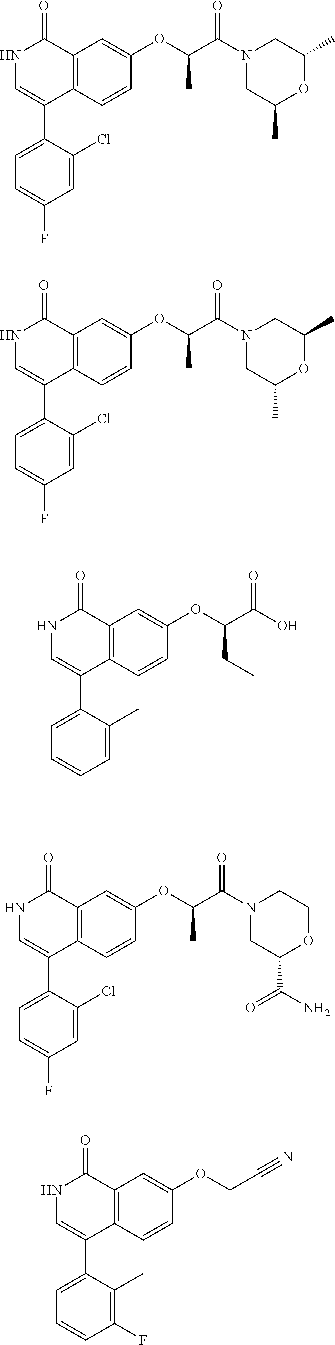 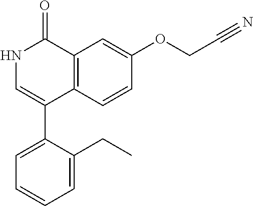 [SMILES: CCc1ccccc1-c1c[nH]c(=O)c2cc(OCC#N)ccc12]
or a pharmaceutically acceptable salt thereof.
